(19) United States
(12) Patent Application Publication
(10) Pub. No.: US 2003/0114666 A1
(43) Pub. Date: Jun. 19, 2003

(54) Antibacterial agents

(76) Inventors: Edmund Lee Ellsworth, Brighton, MI (US); James Alan Kerschen, Wilmington, DE (US); Sharon Anne Powell, Dexter, MI (US); Joseph Peter Sanchez, South Lyon, MI (US); Howard Daniel Hollis Showalter, Ann Arbor, MI (US); Michael Andrew Stier, Ypsilanti, MI (US); Tuan Phong Tran, Canton, MI (US)

Correspondence Address: Heidi M. Berven, Warner-Lambert Company, 2800 Plymouth Road, Ann Arbor, MI, 48105, US

(21) Appl. No.: 10/174,302

(22) Filed: Jun. 18, 2002

Related U.S. Application Data
(60) Provisional application No. 60/299,249, filed on Jun. 19, 2001.
(60) Provisional application No. 60/369,332, filed on Apr. 3, 2002.

(51) Int. Cl. C07D 487/02; C07D 239/72; C 07D 4 3/02

(52) U.S. Cl. 540/600; 544/256; 544/279; 544/285

(57) ABSTRACT

The present invention provides compounds of formula (I):

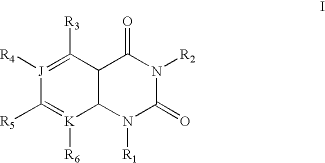

wherein R₁-R₆ and J and K have any of the values defined in the specification, and pharmaceutically acceptable salt thereof, that are useful as antibacterial agents. Also disclosed are pharmaceutical compositions comprising one or more compounds of formula I, processes for preparing compounds of formula I, and intermediates useful for preparing compounds of formula I.

### CROSS REFERENCE TO RELATED APPLICATIONS

This application claims benefit of priority from U.S. Provisional Application No. 60/299,249, filed on Jun. 19, 2001 and 60/369,332, filed on Apr. 3, 2002.

### FIELD OF THE INVENTION

This invention relates to antibacterial agents having a quinazolindione core structure, processes for their preparation, and methods for their use.

### BACKGROUND OF THE INVENTION

Antibiotic resistance is a worldwide problem with catastrophic potential. A Task Force co-chaired by the United States Centers for Disease Control (CDC), Food and Drug Administration (FDA), and National Institutes of Health (NIH) recently addressed this important issue, observing that drug resistant pathogens are a growing menace to all people, regardless of race, age, gender, or socioeconomic background. The Task Force noted that a number of microbes responsible for infections in humans are rapidly developing resistance to existing drugs. For example, according to the Task Force, in the United States alone, up to 30 percent of the Staphylococcus pneumoniae infections (skin, bone, lung, and bloodstream infections) are no longer susceptible to penicillin in some areas. Up to 11 percent of S. pneumoniae are resistant to third generation cephalosporin antibiotics. Significantly, resistance of S. pneumoniae to the fluoroquinolones, a newer class of potent antibiotics, has also been reported.

Exemplified by ciprofloxacin A, the fluoroquinolones are bacterial inhibitors that apparently exert their effect by inhibiting bacterial DNA gyrase and topoisomerase IV.

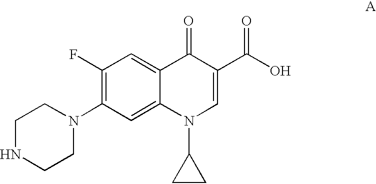

The consequences of antibiotic resistance, particularly fluoroquinolone resistance, can be fatal for some individuals. In a case reported from Denmark, a 62-year-old woman diagnosed with food poisoning from ciprofloxacin-resistant Salmonella died after undergoing antibiotic treatment using that drug.

The dramatic and lethal emergence of antibiotic resistance typified by this and other reports has spurred the U.S. Task Force to call for the implementation of a public health action plan to combat antimicrobial resistance. As a vital component of that plan, there is a need for the development of new products that will prevent the continued emergence of antibiotic resistance generally, and that will prevent and treat colonization and infection of resistant organisms in patients.

### SUMMARY OF THE INVENTION

The present invention provides compounds meeting these and other needs. Accordingly, there is provided a compound of the invention which is a compound of Formula I:

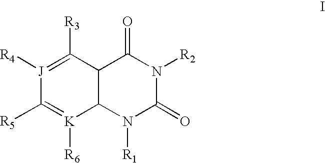

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

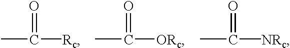

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, R₄, and R₆ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

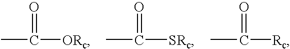

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

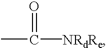

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;

R₁ and R₆ taken together with the atoms to which they are attached form a 5, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

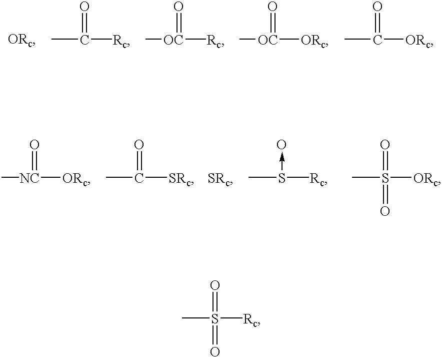

wherein R_{c} is defined as above,

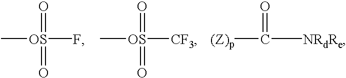

wherein Z is O or N R_{d} and Rₑ are defined as above and p is 0 or 1;

halo,

NO₂,

CN,

NR_{f}R_{g} wherein R_{f} and R_{g} are defined as for Rₐ and R_{b} above;

aryl or fused aryl,

heterocyclic or fused heterocyclic,

heteroaryl or fused heteroaryl,

bicyclic heterocyclic or spiro heterocyclic,

wherein fused aryl, fused heterocyclic, fused heteroaryl, bicyclic heterocyclic, or spiro heterocyclic can be substituted; and

wherein J and K independently are C or N, provided that when J or K is N, R₄ or R₆ is absent at that position.

The invention also provides a compound of Formula II:

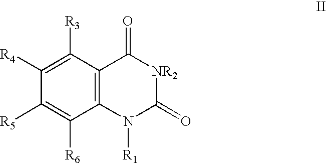

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

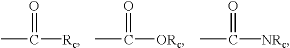

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, R₄, and R₆ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

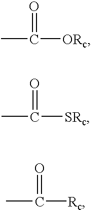

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

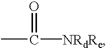

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;

R₁ and R₆ taken together with the atoms to which they are attached to form a 5, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

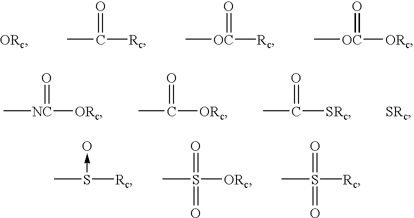

wherein R_{c} is defined as above,

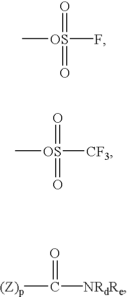

wherein Z is O or N R_{d} and Rₑ are defined as above and p is 0 or 1;

halo,

NO₂,

CN,

NR_{f}R_{g}, wherein R_{f} and R_{g} are defined as for Rₐ and R_{b} above;

aryl or fused aryl,

heterocyclic or fused heterocyclic,

heteroaryl or fused heteroaryl, or

bicyclic heterocyclic or spiro heterocyclic;

wherein fused aryl, fused heterocyclic, fused heteroaryl, bicyclic heterocyclic, or spiro heterocyclic can be substituted.

The present invention also provides a compound of Formula III:

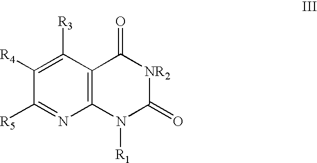

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

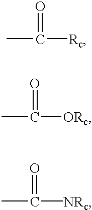

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, and R₄ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

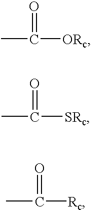

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

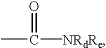

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

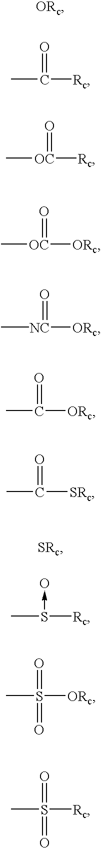

wherein R_{c} is defined as above,

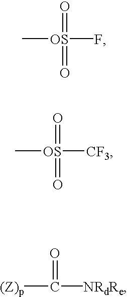

wherein Z is O or N R_{d} and Rₑ are defined as above and p is 0 or 1;

halo,

NO₂,

CN,

NR_{f}R_{g}, wherein R_{f} and R_{g} are defined as for Rₐ and R_{b} above;

aryl or fused aryl,

heterocyclic or fused heterocyclic,

heteroaryl or fused heteroaryl, or

bicyclic heterocyclic or spiro heterocyclic,

wherein fused aryl, fused heterocyclic, fused heteroaryl, bicyclic heterocyclic, or spiro heterocyclic can be substituted.

The present invention also provides a compound of Formula IV:

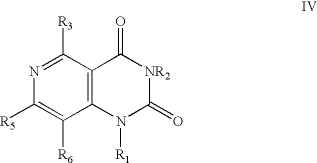

or a pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

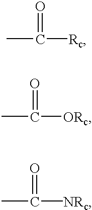

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃ and R₆ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

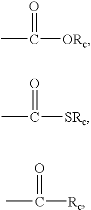

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

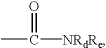

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;

R₁ and R₆ can be taken together with the atoms to which they are attached form a 5, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

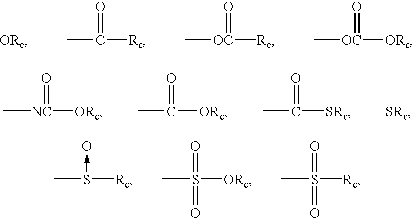

wherein R_{c} is defined as above,

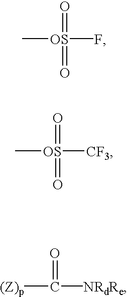

wherein Z is O or N R_{d} and Rₑ are defined as above and p is 0 or 1;

halo,

NO₂,

CN,

NR_{f}R_{g}, wherein R_{f} and R_{g} are defined as for Rₐ and R_{b} above;

aryl or fused aryl,

heterocyclic or fused heterocyclic,

heteroaryl or fused heteroaryl,

bicyclic heterocyclic or spiro heterocyclic,

wherein fused aryl, fused heterocyclic, fused heteroaryl, bicyclic heterocyclic, or spiro heterocyclic can be substituted.

The present invention also provides a compound of Formula V:

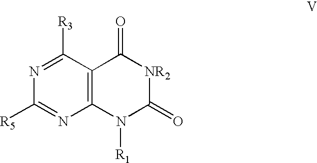

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

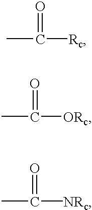

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃ is H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

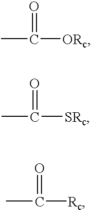

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

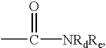

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

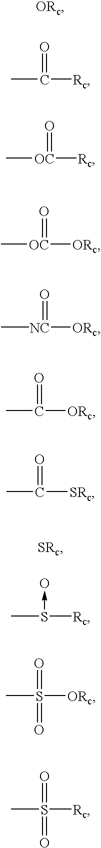

wherein R_{c} is defined as above,

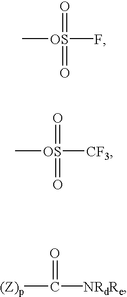

wherein Z is O or N R_{d} and Rₑ are defined as above and p is 0 or 1;

halo,

NO₂,

CN,

NR_{f}R_{g}, wherein R_{f} and R_{g} are defined as for Rₐ and R_{b} above;

aryl or fused aryl,

heterocyclic or fused heterocyclic,

heteroaryl or fused heteroaryl,

bicyclic heterocyclic or spiro heterocyclic,

wherein fused aryl, fused heterocyclic, fused heteroaryl, bicyclic heterocyclic, or spiro heterocyclic can be substituted.

The invention also provides a compound of formula VI:

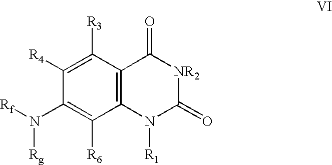

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

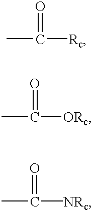

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, R₄, and R₆ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

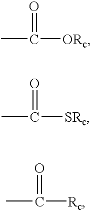

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

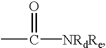

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;

R₁ and R₆ taken together with the atoms to which they are attached form a 5, 6, 7, or 8 membered ring having from 0 to 3 heteroatoms selected from N, O, and S, wherein said ring is optionally substituted by one or more substituents; and

R_{f} and R_{g} are defined as for Rₐand R_{b} above.

The invention also provides a compound of formula VII:

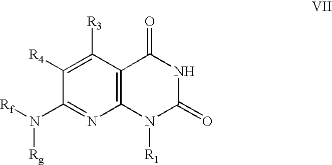

or a tautomer or pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

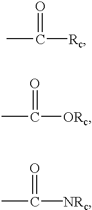

—C—NR_{c}, wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, and R₄ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

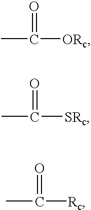

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

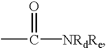

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 6, 7, or 8 membered ring having from 0 to 3 heteroatoms selected from N, O, and S, wherein said ring is optionally substituted by one or more substituents; and

R_{f} and R_{g} are defined as for Rₐand R_{b} above.

The invention also provides a compound of formula VIII:

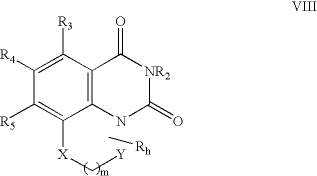

or a tautomer or pharmaceutically acceptable salt thereof wherein:

R₂ is H,

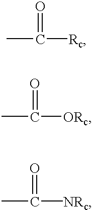

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃ and R₄ independently are H, OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

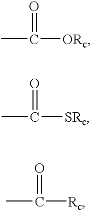

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

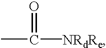

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 6, 7, or 8 membered ring having from 0 to 3 heteroatoms selected from N, O, and S, wherein said ring is optionally substituted by one or more substituents;

R₅ is hydrogen,

C₁-C₇ alkyl and substituted alkyl,

C₂-C₇ alkenyl and substituted alkenyl,

C₂-C₇ alkynyl and substituted alkynyl,

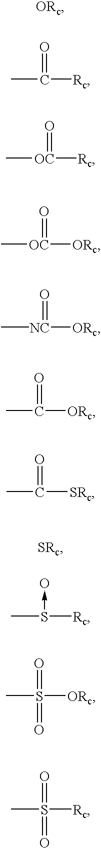

wherein R_{c} is defined as above,

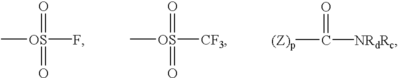

wherein Z is O or N R_{d} and Rₑ are defined as above and p is 0 or 1;

halo,

NO₂,

CN,

NR_{f}R_{g}, wherein R_{f} and R_{g} are defined as for Rₐ and R_{b} above;

aryl or fused aryl,

heterocyclic or fused heterocyclic,

heteroaryl or fused heteroaryl,

bicyclic heterocyclic or spiro heterocyclic,

wherein fused aryl, fused heterocyclic, fused heteroaryl, bicyclic heterocyclic, or spiro heterocyclic can be substituted;

X and Y each independently are O, CH₂, CH(C₁-C₇ alkyl), C(C₁-C₇ alkyl)₂, C(C₃-C₆ cycloalkyl),

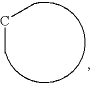

wherein

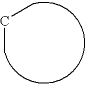

is a C₃-C₆ cycloalkyl, NH, N(C₁-C₇ alkyl), S, SO, or SO₂;

m is 0-14;

Rₕ is H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRⱼRₖ,

wherein Rⱼand Rₖindependently are H,

C₁-C₇ alkyl and substituted alkyl,

C₂-C₇ alkenyl and substituted alkenyl,

C₂-C₇ alkynyl and substituted alkynyl,

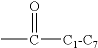

alkyl and substituted alkyl, or

Rⱼ and Rₖ taken together with the nitrogen to which they are attached form a 3- to 7-membered ring containing from 1 to 3 heteroatoms selected from N, O, and S, said ring being unsubstituted or substituted with 1, 2, 3, or 4 substituent groups.

The invention also provides a compound of formula IX:

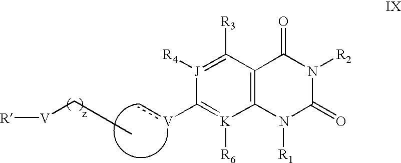

or a pharmaceutically acceptable salt thereof wherein:

R₁ is H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

aryl and substituted aryl,

heterocyclic and substituted heterocyclic,

or heteroaryl and substituted heteroaryl;

R₂ is H,

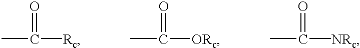

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

R₃, R₄, and R₆ independently are H,

OH,

(O)ₙC₁-C₇ alkyl and substituted alkyl,

(O)ₙC₂-C₇ alkenyl and substituted alkenyl,

(O)ₙC₂-C₇ alkynyl and substituted alkynyl,

wherein n is 0 or 1,

halo,

NO₂,

CN,

NRₐR_{b}, wherein Rₐ and R_{b} are each independently H,

C₁-C₇ alkyl and substituted alkyl,

C₂-C₇ alkenyl and substituted alkenyl,

C₂-C₇ alkynyl and substituted alkynyl,

C₃-C₇ cycloalkyl and substituted cycloalkyl,

C₅-C₈ cycloalkenyl and substituted cycloalkenyl,

aryl and substituted aryl, or

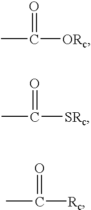

wherein R_{c} is

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl,defined as above;

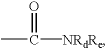

wherein R_{d} and Rₑ are independantly H,

C₁-C₇ alkyl and substituted alkyl,

C₂-C₇ alkenyl and substituted alkenyl,

C₃-C₇ cycloalkyl and substituted cycloalkyl,

aryl and substituted aryl,

heteroaryl and substituted heteroaryl,

heterocycloalkyl and substituted heterocycloalkyl;

aryl and substituted aryl,

heteroaryl and substituted heteroaryl,

heterocycloalkyl and substituted heterocycloalkyl, or

Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;

R₁ and R₆ taken together with the atoms to which they are attached form a 5, 6, 7, or 8 membered ring having from 0 to 3 heteroatoms selected from N, O, and S, wherein said ring is optionally substituted by one or more substituents;

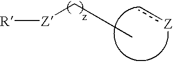

is aryl or fused aryl,

heterocyclic or fused heterocyclic,

heteroaryl or fused heteroaryl,

bicyclic heterocyclic or spiro heterocyclic,

wherein fused aryl, fused heterocyclic, fused heteroaryl, bicyclic heterocyclic, or spiro heterocyclic can be substituted;

V is N, CH, or C, provided that when Z is N or CH, “- -” is absent and when Z is C, “ [symbol] ” is a double bond;

z is 0, 1, 2, or 3;

V′ is O, S, NH₂, NHR″, wherein R″ is C₁-C₇ alkyl and substituted alkyl;

R′ is

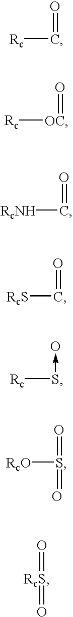

wherein R_{c} is defined as above,

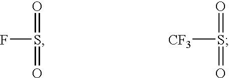

and

wherein J and K independently are C or N, provided that when J or K is N, R₄ or R₆ is absent at that position.

The invention also provides a compound which is

7-(6-amino-3-aza-bicyclo[3.1.0]hex-3-yl)-6-fluoro-3H-1-methylcyclopropyl-1H-quinazoline-2,4-dione (1α, 5α, 6α) hydrochloride,

1-Cyclopropyl-6-fluoro-8-methyl-7-[(R)-3-((S)-1-methylaminoethyl)-pyrrolidin-1-yl]-1H-quinazolinedione,

1-Cyclopropyl-6-fluoro-8-methoxy-7-[(R)-3-((S)-1-methylaminoethyl)-pyrrolidin-1-yl]-1H-quinazolinedione,

7-[(R)-3-((S)-1-Aminoethyl)pyrrolidin-1-yl]-1-cyclopropyl-6-fluoro-8-methyl-1H-quinazolinedione hydrochloride,

1-Cyclopropyl-7-dimethylamino-6-fluoro-8-methyl-1H-quinazolinedione,

7-((S)-3-Amino-pyrrolidin-1-yl)-8-chloro-1-cyclopropyl-6-fluoro-1H-quinazolinedione trifluoroacetic acid,

7-(3-[1-Amino-1-(2-fluorophenyl)methyl]-pyrrolidin-1-yl)-1-cyclopropyl-6-fluoro-6-methyl-1H-quinazolinedione hydrochloride,

1-Cyclopropyl-8-methyl-7-[(R)-3-((S)-1-methylaminoethyl)pyrrolidin-1-yl]-1H-pyrido[4,3-d]pyrimidinedione hydrochloride,

7-((S)-3-Aminopyrrolidin-1-yl)-1-cyclopropyl-6-fluoro-1H-pyrido[2,3-d]pyrimidinedione hydrochloride,

7-[(R)-3-((S)-1-Aminoethyl)pyrrolidin-1-yl]-1-cyclopropyl-6-fluoro-1H-pyrido[2,3-d]pyrimidinedione hydrochloride,

7-((S)-3-Aminopyrrolidin-1-yl)-8-fluoro-5-methyl-5,6-dihydropyrrolo[3,2, 1-ij]quinazoline-1,3-dione hydrochloride,

7-[(R)-3-((S)-1-Aminoethyl)pyrrolidin-1-yl)-8-fluoro-5-methyl-5,6-dihydropyrrolo[3,2,1-i,j] quinazoline-1,3-dione hydrochloride,

8-((S)-3-Aminopyrrolidin-1-yl)-9-fluoro-5-methyl-6,7-dihydropyrido[3,2,1-i,j]quinazoline-1,3-dione hydrochloride,

8-[(R)-3-((S)-1-Aminoethyl)pyrrolidin-1-yl)-9-fluoro-5-methyl-6,7-dihydro-5H-pyrido[3,2,1-ij] quinazoline-1,3-dione hydrochloride,

1-(1-Cyclopropyl-6-fluoro-8-methyldioxo-1,2,3,4-tetrahydroquinazolin-7-yl)cyclopropanecarbonitrile,

1-(1-Cyclopropyl-6-fluoro-8-methyldioxo-1,2,3,4-tetrahydroquinazolin-7-yl)cyclopropanecarboxylic acid amide,

7-Amino-9-[9-(R)-3-((S)-1-aminoethyl)pyrrolidin-1-yl)-8-fluoro-3-methyl-2,3-dihydro-1-oxa-3a,5-diazaphenalene-4,6-dione hydrochloride,

7-((3aR, 6aS)- and (3aS, 6aR)-4-Aminohexahydrocyclopenta[c]pyrrol-2-yl-1-cyclopropyl-6-fluoro-8-methyl-1H-quinazolinedione, hydrochloride,

7-((3aR, 6aS)- and (3aS, 6aR)-4-Aminohexahydrocyclopenta[c]pyrrol-2-yl-1-cyclopropyl-6-fluoro-8-methoxy-1H-quinazolinedione hydrochloride,

7-[3-(1-Amino-2-fluoroethyl)pyrrolidin-1-yl]-1-cyclopropyl-6-fluoro-8-methyl-1H-quinazolinedione hydrochloride,

7-[3-(Aminocyclopropylmethyl)-pyrrolidin-1-yl]-1-cyclopropyl-6-fluoro-8-methyl-1H-quinazolinedione hydrochloride,

7-(3-Aminomethyl-4-fluoromethylpyrrolidin-1-yl)-1-cyclopropyl-6-fluoro-8-methyl-1H-quinazolinedione,

7-(5-Aminomethylthiophen-3-yl)-1-cyclopropyl-6-fluoro-8-methyl-1H-quinazoline-2,4-dione hydrochloride,

1-Cyclopropyl-7-(5,5-difluoro-4-hydroxy-4,5,6,7-tetrahydrobenzo[b]thiophen-2-yl)-6-fluoro-8-methyl-1H-quinazolinedione,

7-(4-Amino-5,5-difluoro-4,5,6,7-tetrahydrobenzo[b]thiophen-2-yl)-1-cyclopropyl-6-fluoro-8-methyl-1H-quinazolinedione hydrochloride,

7-(4-Amino-5,6-dihydro-4H-cyclopenta[b]thiophen-2-yl)-1-cyclopropyl-6-fluoro-8-methyl-1H-quinazolinedione hydrochloride,

7-[(R)-3-(1-Aminocyclopropyl)pyrrolidin-1-yl]-1-cyclopropyl-6-fluoro-8-methyl-1H-quinazolinedione hydrochloride,

7-(4-Amino-5,6-dihydro-4H-4,5,6,7-tetrahydrobenzo[b]-thiophen-7-yl)-1-cyclopropyl-6-fluoro-8-methyl-1H-quinazolinedione hydrochloride,

1-cyclopropyl-6-fluoro-8-methyl-7-(7-methyl-4,5,6,7-tetrahydrothieno[2,3-c]pyridin-2-yl)-1H-quinazolinedione,

1-Cyclopropyl-6-fluoro-8-methyl-7-(4-methyl-5,6-dihydro-4H-thieno[2,3-c]pyrrol-2-yl)-1H-quinazolinedione hydrochloride,

7-[[(3S, 4R)-3-(R)- and (3R, 4S)-3-(S)]-1-amino-2,2,2-trifluoroethyl)-4-hydroxypyrrolidin-1-yl]-1-cyclopropyl-6-fluoro-8-methyl-1H-quinazolinedione,

7-[3-(Aminooxazol-4-ylmethyl)pyrrolidin-1-yl]-1cyclopropyl-6-fluoro-8-methyl-1H-quinazolinedione,

7-(3R, 4S)- and 7-((3S, 4R)-3-Aminomethyl-4-fluoropyrrolidin-1-yl)-1-cyclopropyl-6-fluoro-8-methoxy-1H-quinazoloinedione hydrochloride,

7-(3-Aminohexahydrofuro[2,3-c]pyrrol-5-yl)-1-cyclopropyl-6-fluoro-8-methyl-1H-quinazolinedione hydrochloride,

7-[4-(Aminoethyl)-3,3-dimethylpyrrolidin-1-yl]-1-cylcopropyl-6-fluoro-8-methyl-1H-quinazolinedione hydrochloride,

7-(4-Aminooctahydroisoindol-2-yl)-1-cyclopropyl-6-fluoro-8-methyl-1H-quinazolinedione hydrochloride,

7-[(R)-3-((S)-1-Aminoethyl)pyrrolidin-1-yl]-1-cyclopropyl-6-fluoro-8-fluoromethoxy-1H-quinazolinedione hydrochloride,

7-[(R)-3-((S)-1-Aminoethyl)pyrrolidin-1-yl]-1-cyclopropyl-8-difluoromethyl-6-fluoro-1H-quinazolinedione hydrochloride,

7-[5-(1-Aminocyclopropyl)thiophen-2-yl]-1-cyclopropyl-6-fluoro-8-methyl-1H-quinazolinedione hydrochloride,

7-[(R)-3-(1-Aminocyclopropyl)pyrrolidin-1-yl]-1-cyclopropyl-8-diflouromethoxy-6-fluoro-1H-quinazolinedione,

7-[(R)-3-((S)-1-Aminoethyl)pyrrolidin-1-yl]-1-cyclopropyl-6-fluoro-8-difluoromethoxy-1H-quinazolinedione hydrochloride,

7-[(R)-3-((S)-1-Aminoethyl)pyrrolidin-1-yl]-1-cyclopropyl-6-fluoro-5,8-dimethyl-1H-quinazolinedione hydrochloride,

1-Cyclopropyl-8-difluoromethoxy-7-((R)-1-methyl-2,3-dihydro-1H-isoindol-5-yl)-1H-quinazolinedione hydrochloride,

7-[(R)-3-((S)-1-Aminoethyl)pyrrolidin-1-yl]-1-cyclopropyl-8-difluoromethoxy-1H-quinazolinedione hydrochloride,

7-((3R, 4S)- and (3S, 4R)-3-Aminomethyl-4-trifluoromethylpyrrolidin-1-yl)-1-cyclopropyl-8-difluoromethoxy-6-fluoro-] H-quinazolinedione hydrochloride,

1-Cyclopropyl-6-fluoro-8-methoxy-7-[3(R)-(1(S)-methylaminoethyl)pyrrolidin-]-yl]-1H-quinazolinedione,

1-Cyclopropyl-6-fluoro-8-methyl-7-[3(R)-(] (S)-methylaminoethyl)pyrrolidin-1-yl]-1H-quinazolinedione,

7-(3-Aminopiperidin-1-yl)-1-cyclopropyl-6-fluoro-8-methoxy-1H-quinazolinedione,

1-Cyclopropyl-6-fluoro-8-methoxy-7-(octahydropyrrolo[3,4-c]pyridin-2-yl)-1H-quinazolinedione,

7-((S)-3-Aminopyrrolidin-1-yl)-1-cyclopropyl-6-fluoro-8-methyl-1H-quinazolinedione,

7-(3-Aminopiperidin-1-yl)-1-cyclopropyl-6-fluoro-8-methyl-1H-quinazolinedione,

1-Cyclopropyl-6-fluoro-8-methyl-7-(octahydropyrrolo[3,4-c]pyridin-2-yl)-1H-quinazolinedione,

7-(3(S)-Aminopyrrolidin-1-yl)-1-cyclopropyl-6-fluoro-8-methoxy-1H-quinazolinedione,

7-(3-Aminomethyl-3-methylpyrrolidin-1-yl)-1-cyclopropyl-6-fluoro-8-methoxy-1H-quinazolinedione,

7-(3-Aminomethylpyrrolidin-1-yl)-1-cyclopropyl-6-fluoro-8-methoxy-1H-quinazolinedione,

7-[3(R)-(1-Amino-1-methylethyl)-pyrrolidin-1-yl]-1-cyclopropyl-6-fluoro-8-methoxy-1H-quinazolinedione,

7-(3-Aminomethylpiperidin-1-yl)-1-cyclopropyl-6-fluoro-8-methoxy-1H-quinazolinedione,

7-(3-Aminomethyl-3-benzylpyrrolidin-1-yl)-1-cyclopropyl-6-fluoro-8-methoxy-1H-quinazolinedione,

1-Cyclopropyl-6-fluoro-8-methoxy-7-(octahydropyrrolo[3,4-b]pyridin-6-yl)-1H-quinazolinedione,

7-(1-Amino-5-aza-spiro[2.4]hept-5-yl)-1-cyclopropyl-6-fluoro-8-methoxy-1H-quinazolinedione,

7-(3-Aminomethyl-3-methylpyrrolidin-1-yl)-1-cyclopropyl-6-fluoro-8-methyl-1H-quinazolinedione,

7-[3(R)-(1-Amino-1-methylethyl)pyrrolidin-1-yl]-1-cyclopropyl-6-fluoro-8-methyl-1H-quinazolinedione,

1-Cyclopropyl-6-fluoro-8-methyl-7-(octahydropyrrolo[3,4-b]pyridin-6-yl)-1H-quinazolinedione,

7-(3a-Aminomethyloctahydroisoindol-2-yl)-1-cyclopropyl-6-fluoro-8-methyl-1H-quinazolinedione,

7-(3S, 4R)- and 7-((3R, 4S)-3-Amino-4-fluoromethylpyrrolidin-1-yl)-1-cyclopropyl-6-fluoro-8-methoxy-1H-quinazolinedione,

1-Cyclopropyl-7-[3(R)-(1-ethylaminoethyl)pyrrolidin-1-yl]-6-fluoro-8-methoxy-1H-quinazolinedione,

7-(3a-Aminomethyloctahydroisoindol-2-yl)-1-cyclopropyl-6-fluoro-8-methoxy-1H-quinazolinedione,

7-(3S, 4R)- and 7-((3R, 4S)-3-Amino-4-fluoromethylpyrrolidin-1-yl)-1-cyclopropyl-6-fluoro-8-methyl-1H-quinazolinedione,

1-Cyclopropyl-7-[(R)-3-((S)-1-ethylaminoethyl)pyrrolidin-1-yl]-6-fluoro-8-methyl-1H-quinazolinedione,

7-[(R)-3-((S)-1-Aminoethyl)pyrrolidin-1-yl]-1-cyclopropyl-6-fluoro-8-methoxy-5-methyl-1H-quinazolinedione hydrochloride; or

a pharmaceutically acceptable salt thereof.

The invention also provides a compound of the invention which is:

7-[3-Aminocyclopropyl)-4-trifluoromethylpyrrolidin-1-yl]-1-cyclopropyl-6-fluoro-8-methoxy-5-methyl-1H-quinazolinedione;

1-Cyclopropyl-6-fluoro-7-(3-hydroxymethylpyrrolidin-1-yl)-8-methoxy-5-methyl-1H-quinazolinedione;

1-Cyclopropyl-6-fluoro-8-methoxy-5-methyl-7-pyrrolidin-1-yl-1H-quinazolinedione;

7-[3-Aminopyrrolidin-1-yl]-1-cyclopropyl-6-fluoro-8-methoxy-5-methyl-1H-quinazolinedione;

7-[3-(2-Amino-1-hydroxyethyl)pyrrolidin-1-yl]-1-cyclopropyl-6-fluoro-8-methoxy-5-methyl-1H-quinazolinedione;

7-[3-(2-Amino-1-hydroxyethyl)-4-fluoropyrrolidin-1-yl]-1-cyclopropyl-6-fluoro-8-methoxy-5-methyl-1H-quinazolinedione;

1-Cyclopropyl-6-fluoro-8-methoxy-5-methyl-7-morpholin-4-yl-1H-quinazolinedione;

1-Cyclopropyl-6-fluoro-8-methoxy-5-methyl-7-piperazin-1-yl-1H-quinazolinedione;

1-Cyclopropyl-7-{3-[(2,4-difluorophenyl)hydroxymethyl]-4-fluoropyrrolidin-1-yl}-6-fluoro-8-methoxy-5-methyl-1H-quinazolinedione;

1-Cyclopropyl-7-{3-[(4-fluorophenyl)hydroxymethyl]-4-fluoropyrrolidin-1-yl}-6-fluoro-8-methoxy-5-methyl-1H-quinazolinedione;

1-Cyclopropyl-6-fluoro-7-(4-hydroxyoctahydroisoindol-2-yl)-8-methoxy-5-methyl-1H-quinazolinedione;

1-Cyclopropyl-6-fluoro-7-(4-hydroxyhexahydrocyclopent[c]pyrrol-2-yl)-8-methoxy-5-methyl-1H-quinazolinedione;

5-Amino-7-[3-aminocyclopropyl)-4-trifluoromethylpyrrolidin-1-yl]-1-cyclopropyl-6-fluoro-8-methoxy-1H-quinazolinedione;

5-Amino-1-cyclopropyl-6-fluoro-7-(3-hydroxymethylpyrrolidin-1-yl)-8-methoxy-1H-quinazolinedione;

5-Amino-1-cyclopropyl-6-fluoro-8-methoxy-7-pyrrolidin-1-yl-1H-quinazolinedione;

5-Amino-7-[3-aminopyrrolidin-1-yl-1-cyclopropyl-6-fluoro-8-methoxy-1H-quinazolinedione;

5-Amino-7-[3-(2-amino-1-hydroxyethyl)pyrrolidin-1-yl]-1-cyclopropyl-6-fluoro-8-methoxy-1H-quinazolinedione;

5-Amino-7-[3-(2-amino-1-hydroxyethyl)-4-fluoropyrrolidin-1-yl]-1-cyclopropyl-6-fluoro-8-methoxy-1H-quinazolinedione;

5-Amino-1-cyclopropyl-6-fluoro-8-methoxy-7-morpholin-4-yl-1H-quinazolinedione;

5-Amino-1-cyclopropyl-6-fluoro-8-methoxy-7-piperazin-1-yl-1H-quinazolinedione;

5-Amino-1-cyclopropyl-7-{3-[(2,4-difluorophenyl)hydroxymethyl]-4-fluoropyrrolidin-1-yl}-6-fluoro-8-methoxy-1H-quinazolinedione;

5-Amino-1-cyclopropyl-7-{3-[(4-fluorophenyl)hydroxymethyl]-4-fluoropyrrolidin-1-yl}-6-fluoro-8-methoxy-1H-quinazolinedione;

5-Amino-1-cyclopropyl-6-fluoro-7-(4-hydroxyoctahydroisoindol-2-yl)-8-methoxy-1H-quinazolinedione;

5-Amino-1-cyclopropyl-6-fluoro-7-(4-hydroxyhexahydrocyclopent[c]pyrrol-2-yl)-8-methoxy-1H-quinazolinedione;

7-[3-Aminocyclopropyl)-4-trifluoromethylpyrrolidin-1-yl]-1-cyclopropyl-6-fluoro-5-hydroxy-8-methoxy-1H-quinazolinedione;

1-Cyclopropyl-6-fluoro-7-(3-hydroxymethylpyrrolidin-1-yl)-5-hydroxy-8-methoxy-1H-quinazolinedione;

1-Cyclopropyl-6-fluoro-5-hydroxy-8-methoxy-7-pyrrolidin-1-yl-1H-quinazolinedione;

7-[3-Aminopyrrolidin-1-yl]-1-cyclopropyl-6-fluoro-5-hydroxy-8-methoxy-1H-quinazolinedione;

7-[3-(2-Amino-1-hydroxyethyl)pyrrolidin-1-yl]-11-cyclopropyl-6-fluoro-5-hydroxy-8-methoxy-1H-quinazolinedione;

7-[3-(2-Amino-1-hydroxyethyl)-4-fluoropyrrolidin-1-yl]-1-cyclopropyl-6-fluoro-5-hydroxy-8-methoxy-1H-quinazolinedione;

1-Cyclopropyl-6-fluoro-5-hydroxy-8-methoxy-7-morpholin-4-yl-1H-quinazolinedione;

1-Cyclopropyl-6-fluoro-5-hydroxy-8-methoxy-7-piperazin-1-yl-1H-quinazolinedione;

1-Cyclopropyl-7-{3-[(2,4-difluorophenyl)hydroxymethyl]-4-fluoropyrrolidin-1-yl}-6-fluoro-5-hydroxy-8-methoxy-1H-quinazolinedione;

1-Cyclopropyl-7-{3-[(4-fluorophenyl)hydroxymethyl]-4-fluoropyrrolidin-1-yl}-6-fluoro-5-hydroxy-8-methoxy-1H-quinazolinedione;

1-Cyclopropyl-6-fluoro-7-(4-hydroxyoctahydroisoindol-2-yl)-5-hydroxy-8-methoxy-1H-quinazolinedione;

1-Cyclopropyl-6-fluoro-7-(4-hydroxyhexahydrocyclopent[c]pyrrol-2-yl)-5-hydroxy-8-methoxy-1H-quinazolinedione;

7-[3-(1-Aminocyclopropyl)-4-trifluoromethylpyrrolidin-1-yl]-1-cyclopropyl-5-difluoromethyl-6-fluoro-8-methoxy-1H-quinazolinedione;

1-Cyclopropyl-5-difluoromethyl-6-fluoro-7-(3-hydroxymethylpyrrolidin-1-yl)-8-methoxy-1H-quinazolinedione;

7-(3-Aminopyrrolidin-1-yl)-1-cyclopropyl-5-difluoromethyl-6-fluoro-8-methoxy-1H-quinazolinedione;

7-[3-(1-Amino-2-hydroxyethyl)-4-fluoropyrrolidin-1-yl]-1-cyclopropyl-5-difluoromethyl-6-fluoro-8-methoxy-1H-quinazolinedione;

1-Cyclopropyl-5-difluoromethyl-6-fluoro-8-methoxy-7-pyrrolidin-1-yl-1H-quinazolinedione;

7-[3-(2-Amino-1-hydroxyethyl)pyrrolidin-1-yl]-1-cyclopropyl-5-difluoromethyl-6-fluoro-8-methoxy-1H-quinazolinedione;

1-Cyclopropyl-5-difluoromethyl-6-fluoro-8-methoxy-7-piperazin-1-yl-1H-quinazolinedione;

1-Cyclopropyl-5-difluoromethyl-6-fluoro-8-methoxy-7-morpholin-4-yl-1H-quinazolinedione;

1-Cyclopropyl-5-difluoromethyl-6-fluoro-7-{3-fluoro-4-[(4-fluorophenyl)-hydroxymethyl]pyrrolidin-1-yl}-8-methoxy-1H-quinazolinedione;

1-Cyclopropyl-5-difluoromethyl-7-{3-[(2,4-difluorophenyl)hydroxymethyl]-4-fluoropyrrolidin-1-yl}-6-fluoro-8-methoxy-1H-quinazolinedione;

1-Cyclopropyl-5-difluoromethyl-6-fluoro-7-(4-hydroxyoctahydroisoindol-2-yl)-8-methoxy-1H-quinazolinedione;

1-Cyclopropyl-5-difluoromethyl-6-fluoro-7-(4-hydroxyhexahydro-cyclopenta[c]pyrrol-2-yl)-8-methoxy-1H-quinazolinedione;

7-(4-Aminooctahydrocyclohepta[c]pyrrol-2-yl)-1-cyclopropyl-5-difluoromethyl-6-fluoro-8-methoxy-1H-quinazolinedione;

1-Cyclopropyl-6-fluoro-7-(3-hydroxymethylpyrrolidin-1-yl)-5,8-dimethyl-1H-quinazolinedione;

7-(3-Aminopyrrolidin-1-yl)-1-cyclopropyl-6-fluoro-5,8-dimethyl-1H-quinazolinedione;

7-[3-(1-Amino-2-hydroxyethyl)-4-fluoropyrrolidin-1-yl]-1-cyclopropyl-6-fluoro-5,8-dimethyl-1H-quinazolinedione;

1-Cyclopropyl-6-fluoro-5,8-dimethyl-7-pyrrolidin-1-yl-1H-quinazolinedione;

7-[3-(2-Amino-1-hydroxyethyl)pyrrolidin-1-yl]-1-cyclopropyl-6-fluoro-5,8-dimethyl-1H-quinazolinedione;

1-Cyclopropyl-6-fluoro-5,8-dimethyl-7-piperazin-1-yl-1H-1.0 quinazolinedione;

1-Cyclopropyl-6-fluoro-5,8-dimethyl-7-morpholin-4-yl-1H-quinazolinedione;

1-Cyclopropyl-6-fluoro-7-{3-fluoro-4-[(4-fluorophenyl)hydroxymethyl]-pyrrolidin-1-yl}-5,8-dimethyl-1H-quinazolinedione;

1-Cyclopropyl-7-{3-[(2,4-difluorophenyl)hydroxymethyl]-4-fluoropyrrolidin-1-yl}-6-fluoro-5,8-dimethyl-1H-quinazolinedione;

1-Cyclopropyl-6-fluoro-7-(4-hydroxyoctahydroisoindol-2-yl)-5,8-dimethyl-1H-quinazolinedione;

1-Cyclopropyl-6-fluoro-7-(4-hydroxyhexahydrocyclopenta[c]pyrrol-2-yl)-5,8-dimethyl-1H-quinazolinedione;

1-Cyclopropyl-6-fluoro-7-(4-hydroxyoctahydrocyclohepta[c]pyrrol-2-yl)-5,8-dimethyl-1H-quinazolinedione;

7-[3-(1-Aminocyclopropyl)-4-trifluoromethylpyrrolidin-1-yl]-1-cyclopropyl-6-fluoro-5-methoxy-8-methyl-1H-quinazolinedione;

1-Cyclopropyl-6-fluoro-7-(3-hydroxymethylpyrrolidin-1-yl)-5-methoxy-8-methyl-1H-quinazolinedione;

7-(3-Aminopyrrolidin-1-yl)-1-cyclopropyl-6-fluoro-5-methoxy-8-methyl-1H-quinazolinedione;

7-[3-(1-Amino-2-hydroxyethyl)-4-fluoropyrrolidin-1-yl]-1-cyclopropyl-6-fluoro-5-methoxy-8-methyl-1H-quinazolinedione;

1-Cyclopropyl-6-fluoro-5-methoxy-8-methyl-7-pyrrolidin-1-yl-1H-quinazolinedione;

7-[3-(2-Amino-1-hydroxyethyl)pyrrolidin-1-yl]-1-cyclopropyl-6-fluoro-5-methoxy-8-methyl-1H-quinazolinedione;

1-Cyclopropyl-6-fluoro-5-methoxy-8-methyl-7-piperazin-1-yl-1H-quinazolinedione;

1-Cyclopropyl-6-fluoro-5-methoxy-8-methyl-7-morpholin-4-yl-1H-quinazolinedione;

1-Cyclopropyl-6-fluoro-7-{3-fluoro-4-[(4-fluorophenyl)hydroxymethyl]-pyrrolidin-1-yl}-5-methoxy-8-methyl-1H-quinazolinedione;

1-Cyclopropyl-7-{3-[(2,4-difluorophenyl)hydroxymethyl]-4-fluoropyrrolidin-1-yl}-6-fluoro-5-methoxy-8-methyl-1H-quinazolinedione;

1-Cyclopropyl-6-fluoro-7-(4-hydroxy-octahydro-isoindol-2-yl)-5-methoxy-8-methyl-1H-quinazolinedione;

1-Cyclopropyl-6-fluoro-7-(4-hydroxyhexahydrocyclopenta[c]pyrrol-2-yl)-5-methoxy-8-methyl-1H-quinazolinedione;

1-Cyclopropyl-6-fluoro-7-(4-hydroxyoctahydrocyclohepta[c]pyrrol-2-yl)-5-methoxy-8-methyl-1H-quinazolinedione;

7-(3-Aminomethylpyrrolidin-1-yl)-1-cyclopropyl-6-fluoro-8-methoxy-5-methyl-1H-quinazolinedione;

7-[3-(1-Aminocyclopropyl)pyrrolidin-1-yl]-1-cyclopropyl-6-fluoro-8-methoxy-5-methyl-1H-quinazolinedione;

7-[3-(1-Amino-1-methylethyl)pyrrolidin-1-yl]-1-cyclopropyl-6-fluoro-8-methoxy-5-methyl-1H-quinazolinedione;

7-[3-(1-Amino-2-fluoroethyl)pyrrolidin-1-yl]-1-cyclopropyl-6-fluoro-8-methoxy-5-methyl-1H-quinazolinedione;

7-[3-(1-Amino-2,2-difluoroethyl)pyrrolidin-1-yl]-1-cyclopropyl-6-fluoro-8-methoxy-5-methyl-1H-quinazolinedione;

7-[3-(1-Amino-2,2,2-trifluoroethyl)pyrrolidin-1-yl]-1-cyclopropyl-6-fluoro-8-methoxy-5-methyl-1H-quinazolinedione;

7-[3-(1-Aminoethyl)-4-fluoropyrrolidin-1-yl]-1-cyclopropyl-6-fluoro-8-methoxy-5-methyl-1 H-quinazolinedione;

7-{3-[Amino-(2,6-difluorophenyl)methyl]pyrrolidin-1-yl}-1-cyclopropyl-6-fluoro-8-methoxy-5-methyl-1H-quinazolinedione;

7-(3-Aminomethyl-4-fluoromethylpyrrolidin-1-yl)-1-cyclopropyl-6-fluoro-8-methoxy-5-methyl-1H-quinazolinedione;

7-[3-(Aminothiazol-2-yl-methyl)pyrrolidin-1-yl]-1-cyclopropyl-6-fluoro-8-methoxy-5-methyl-1H-quinazolinedione;

7-[3-(Aminocyclopropylmethyl)pyrrolidin-1-yl]-1-cyclopropyl-6-fluoro-8-methoxy-5-methyl-1H-quinazolinedione;

7-(4-Aminooctahydroisoindol-2-yl)-1-cyclopropyl-6-fluoro-8-methoxy-5-methyl-1H-quinazolinedione;

7-(4-Aminooctahydrocyclohepta[c]pyrrol-2-yl)-1-cyclopropyl-6-fluoro-8-methoxy-5-methyl-1H-quinazolinedione;

1-Cyclopropyl-6-fluoro-7-[3-(1-hydroxycyclopropyl)pyrrolidin-1-yl]-8-methoxy-5-methyl-1H-quinazolinedione;

7-(4-Aminohexahydrocyclopenta[c]pyrrol-2-yl)-1-cyclopropyl-6-fluoro-8-methoxy-5-methyl-1H-quinazolinedione;

7-[3-(Aminooxazol-4-yl-methyl)pyrrolidin-1-yl]-1-cyclopropyl-6-fluoro-8-methoxy-5-methyl-1H-quinazolinedione;

5-Amino-7-(3-aminomethylpyrrolidin-1-yl)-1-cyclopropyl-6-fluoro-8-methoxy-1H-quinazolinedione;

5-Amino-7-[3-(1-aminocyclopropyl)pyrrolidin-1-yl]-1-cyclopropyl-6-fluoro-8-methoxy-1H-quinazolinedione;

5-Amino-7-[3-(1-amino-1-methylethyl)pyrrolidin-1-yl]-1-cyclopropyl-6-fluoro-8-methoxy-1H-quinazolinedione;

5-Amino-7-[3-(1-amino-2-fluoroethyl)pyrrolidin-1-yl]-1-cyclopropyl-6-fluoro-8-methoxy-1H-quinazolinedione;

5-Amino-7-[3-(1-amino-2,2-difluoroethyl)pyrrolidin-1-yl]-1-cyclopropyl-6-fluoro-8-methoxy-1H-quinazolinedione;

5-Amino-7-[3-(1-amino-2,2,2-trifluoroethyl)pyrrolidin-1-yl]-1-cyclopropyl-6-fluoro-8-methoxy-1H-quinazolinedione;

5-Amino-7-[3-(1-aminoethyl)-4-fluoropyrrolidin-1-yl]-1-cyclopropyl-6-fluoro-8-methoxy-1H-quinazolinedione;

5-Amino-7-{3-[amino-(2,6-difluorophenyl)methyl]pyrrolidin-1-yl}-1-cyclopropyl-6-fluoro-8-methoxy-1H-quinazolinedione;

5-Amino-7-(3-aminomethyl-4-fluoromethylpyrrolidin-1-yl)-1-cyclopropyl-6-fluoro-8-methoxy-1H-quinazolinedione;

5-Amino-7-[3-(aminothiazol-2-yl-methyl)pyrrolidin-1-yl]-1-cyclopropyl-6-fluoro-8-methoxy-1H-quinazolinedione;

5-Amino-7-[3-(aminocyclopropylmethyl)pyrrolidin-1-yl]-1-cyclopropyl-6-fluoro-8-methoxy-1H-quinazolinedione,

5-Amino-7-(4-aminooctahydroisoindol-2-yl)-1-cyclopropyl-6-fluoro-8-methoxy-1H-quinazolinedione;

5-Amino-7-(4-aminooctahydrocyclohepta[c]pyrrol-2-yl)-1-cyclopropyl-6-fluoro-8-methoxy-1H-quinazolinedione;

5-Amino-1-cyclopropyl-6-fluoro-7-[3-(1-hydroxycyclopropyl)pyrrolidin-1-yl]-8-methoxy-1H-quinazolinedione;

5-Amino-7-(4-aminohexahydrocyclopenta[c]pyrrol-2-yl)-1-cyclopropyl-6-fluoro-8-methoxy-1H-quinazolinedione;

5-Amino-7-[3-(aminooxazol-4-yl-methyl)pyrrolidin-1-yl]-1-cyclopropyl-6-fluoro-8-methoxy-1H-quinazolinedione;

7-(3-Aminomethylpyrrolidin-1-yl)-1-cyclopropyl-5-difluoromethyl-6-fluoro-8-methyl-1H-quinazolinedione;

7-[3-(1-Aminocyclopropyl)pyrrolidin-1-yl]-1-cyclopropyl-5-difluoromethyl-6-fluoro-8-methyl-1H-quinazolinedione;

7-[3-(1-Amino-1-methylethyl)pyrrolidin-1-yl]-1-cyclopropyl-5-difluoromethyl-6-fluoro-8-methyl-1H-quinazolinedione;

7-[3-(1-Amino-2-fluoroethyl)pyrrolidin-1-yl]-1-cyclopropyl-5-difluoromethyl-6-fluoro-8-methyl-1H-quinazolinedione;

7-[3-(1-Amino-2,2-difluoroethyl)pyrrolidin-1-yl]-1-cyclopropyl-5-difluoromethyl-6-fluoro-8-methyl-1H-quinazolinedione;

7-[3-(1-Amino-2,2,2-trifluoroethyl)pyrrolidin-1-yl]-1-cyclopropyl-5-difluoromethyl-6-fluoro-8-methyl-1H-quinazolinedione;

7-[3-(1-Amino-ethyl)-4-fluoro-pyrrolidin-1-yl]-1-cyclopropyl-5-difluoromethyl-6-fluoro-8-methyl-1H-quinazolinedione;

1-Cyclopropyl-5-difluoromethyl-7-{3-[(2,6-difluorophenyl)hydroxymethyl]-pyrrolidin-1-yl}-6-fluoro-8-methyl-1H-quinazolinedione;

7-(3-Aminomethyl-4-fluoromethylpyrrolidin-1-yl)-1-cyclopropyl-5-difluoromethyl-6-fluoro-8-methyl-1H-quinazolinedione;

7-[3-(Aminothiazol-2-yl-methyl)pyrrolidin-1-yl]-1-cyclopropyl-5-difluoromethyl-6-fluoro-8-methyl-1H-quinazolinedione;

7-[3-(Amino-cyclopropyl-methyl)-pyrrolidin-1-yl]-1-cyclopropyl-5-difluoromethyl-6-fluoro-8-methyl-1H-quinazolinedione;

1-Cyclopropyl-5-difluoromethyl-6-fluoro-7-[3-(1-hydroxycyclopropyl)-pyrrolidin-1-yl]-8-methyl-1H-quinazolinedione;

7-[3-(Aminooxazol-4-ylmethyl)pyrrolidin-1-yl]-1-cyclopropyl-5-difluoromethyl-6-fluoro-8-methyl-1H-quinazolinedione;

7-(3-Aminomethylpyrrolidin-1-yl)-1-cyclopropyl-6-fluoro-5,8-dimethyl-1H-quinazolinedione;

7-[3-(1-Aminocyclopropyl)pyrrolidin-1-yl]-1-cyclopropyl-6-fluoro-5,8-dimethyl-1H-quinazolinedione;

7-[3-(1-Amino-2-fluoroethyl)pyrrolidin-1-yl]-1-cyclopropyl-6-fluoro-5,8-dimethyl-1H-quinazolinedione;

7-[3-(1-Amino-2,2-difluoroethyl)pyrrolidin-1-yl]-1-cyclopropyl-6-fluoro-5,8-dimethyl-1H-quinazolinedione;

7-[3-(1-Amino-2,2,2-trifluoroethyl)pyrrolidin-1-yl]-1-cyclopropyl-6-fluoro-5,8-dimethyl-1H-quinazolinedione;

1-Cyclopropyl-7-{3-[(2,6-difluorophenyl)hydroxymethyl]pyrrolidin-1-yl}-6-fluoro-5,8-dimethyl-1H-quinazolinedione;

7-(3-Aminomethyl-4-fluoromethylpyrrolidin-1-yl)-1-cyclopropyl-6-fluoro-5,8-dimethyl-1H-quinazolinedione;

7-[3-(Aminothiazol-2-ylmethyl)pyrrolidin-1-yl]-1-cyclopropyl-6-fluoro-5,8-dimethyl-1H-quinazolinedione;

7-[3-(Aminocyclopropyl methyl)pyrrolidin-1-yl]-1-cyclopropyl-6-fluoro-5,8-dimethyl-1H-quinazolinedione;

1-Cyclopropyl-6-fluoro-7-[3-(1-hydroxycyclopropyl)pyrrolidin-1-yl]-5,8-dimethyl-1H-quinazolinedione;

7-[3-(Aminooxazol-4-ylmethyl)pyrrolidin-1-yl]-1-cyclopropyl-6-fluoro-5,8-dimethyl-1H-quinazolinedione;

5-Amino-7-(3-aminomethylpyrrolidin-1-yl)-1-cyclopropyl-6-fluoro-8-methyl-1H-quinazolinedione;

5-Amino-7-[3-(1-aminocyclopropyl)pyrrolidin-1-yl]-1-cyclopropyl-6-fluoro-8-methyl-1H-quinazolinedione;

5-Amino-7-[3-(1-amino-1-methylethyl)pyrrolidin-1-yl]-1-cyclopropyl-6-fluoro-8-methyl-1H-quinazolinedione;

5-Amino-7-[3-(1-amino-2,2,2-trifluoroethyl)pyrrolidin-1-yl]-1-cyclopropyl-6-fluoro-8-methyl-1H-quinazolinedione;

5-Amino-7-[3-(1-amino-2-fluoroethyl)pyrrolidin-1-yl]-1-cyclopropyl-6-fluoro-8-methyl-1H-quinazolinedione;

5-Amino-7-[3-(1-aminoethyl)-4-fluoropyrrolidin-1-yl]-1-cyclopropyl-6-fluoro-8-methyl-1H-quinazolinedione;

5-Amino-7-[3-(1-amino-2,2-difluoroethyl)pyrrolidin-1-yl]-1-cyclopropyl-6-fluoro-8-methyl-1H-quinazolinedione;

5-Amino-1-cyclopropyl-7-{3-[(2,6-difluorophenyl)hydroxymethyl]pyrrolidin-1-yl}-6-fluoro-8-methyl-1H-quinazolinedione;

5-Amino-7-(3-aminomethyl-4-fluoromethylpyrrolidin-1-yl)-1-cyclopropyl-6-fluoro-8-methyl-1H-quinazolinedione;

5-Amino-7-[3-(aminothiazol-2-ylmethyl)pyrrolidin-1-yl]-1-cyclopropyl-6-fluoro-8-methyl-1H-quinazolinedione;

5-Amino-1-cyclopropyl-6-fluoro-7-[3-(1-hydroxycyclopropyl)pyrrolidin-1-yl]-8-methyl-1H-quinazolinedione;

5-Amino-7-[3-(aminooxazol-4-ylmethyl)pyrrolidin-1-yl]-1-cyclopropyl-6-fluoro-8-methyl-1H-quinazolinedione;

7-[3-(1-Amino-2-fluoroethyl)pyrrolidin-1-yl]-1-cyclopropyl-6-fluoro-5-hydroxy-8-methyl-1H-quinazolinedione;

7-[3-(1-Amino-2,2-difluoroethyl)pyrrolidin-1-yl]-1-cyclopropyl-6-fluoro-5-hydroxy-8-methyl-1H-quinazolinedione;

7-[3-(1-Amino-2,2,2-trifluoroethyl)pyrrolidin-1-yl]-1-cyclopropyl-6-fluoro-5-hydroxy-8-methyl-1H-quinazolinedione;

7-[3-(Aminothiazol-2-ylmethyl)pyrrolidin-1-yl]-1-cyclopropyl-6-fluoro-5-hydroxy-8-methyl-1H-quinazolinedione;

1-Cyclopropyl-6-fluoro-5-hydroxy-7-[3-(1-hydroxycyclopropyl)pyrrolidin-1-yl]-8-methyl-1H-quinazolinedione;

7-[3-(Aminooxazol-4-ylmethyl)pyrrolidin-1-yl]-1-cyclopropyl-6-fluoro-5-hydroxy-8-methyl-1H-quinazolinedione;

7-[3-(1-Aminocyclopropyl)pyrrolidin-1-yl]-1-cyclopropyl-6-fluoro-5-methoxy-8-methyl-1H-quinazolinedione;

7-[3-(1-Amino-2-fluoroethyl)pyrrolidin-1-yl]-1-cyclopropyl-6-fluoro-5-methoxy-8-methyl-1H-quinazolinedione;

7-(3-Aminomethylpyrrolidin-1-yl)-1-cyclopropyl-6-fluoro-5-methoxy-8-methyl-1H-quinazolinedione;

7-[3-(1-Amino-2,2-difluoroethyl)pyrrolidin-1-yl]-1-cyclopropyl-6-fluoro-5-methoxy-8-methyl-1H-quinazolinedione;

7-[3-(1-Amino-2,2,2-trifluoroethyl)pyrrolidin-1-yl]-1-cyclopropyl-6-fluoro-5-methoxy-8-methyl-1H-quinazolinedione;

7-[3-(1-Aminoethyl)-4-fluoropyrrolidin-1-yl]-1-cyclopropyl-6-fluoro-5-methoxy-8-methyl-1H-quinazolinedione;

1-Cyclopropyl-7-{3-[(2,6-difluorophenyl)hydroxymethyl]pyrrolidin-1-yl}-6-fluoro-5-methoxy-8-methyl-1H-quinazolinedione;

7-(3-Aminomethyl-4-fluoromethylpyrrolidin-1-yl)-1-cyclopropyl-6-fluoro-5-methoxy-8-methyl-1H-quinazolinedione;

7-[3-(Aminothiazol-2-ylmethyl)pyrrolidin-1-yl]-1-cyclopropyl-6-fluoro-5-methoxy-8-methyl-1H-quinazolinedione;

1-Cyclopropyl-6-fluoro-7-[3-(1-hydroxycyclopropyl)pyrrolidin-1-yl]-5-methoxy-8-methyl-1H-quinazolinedione;

7-[3-(Aminooxazol-4-ylmethyl)pyrrolidin-1-yl]-1-cyclopropyl-6-fluoro-5-methoxy-8-methyl-1H-quinazolinedione;

7-(4-Amino-5,5-difluorooctahydroisoindol-2-yl)-1-cyclopropyl-5-difluoromethyl-6-fluoro-8-methyl-1H-quinazolinedione;

7-(4-Amino-5,5-difluorohexahydrocyclopenta[c]pyrrol-2-yl)-1-cyclopropyl-5-difluoromethyl-6-fluoro-8-methyl-1H-quinazolinedione;

7-(5,5-Difluoro-4-hydroxyoctahydroisoindol-2-yl)-1-cyclopropyl-5-difluoromethyl-6-fluoro-8-methyl-1H-quinazolinedione;

7-(5,5-Difluoro-4-hydroxyhexahydrocyclopenta[c]pyrrol-2-yl)-1-cyclopropyl-5-difluoromethyl-6-fluoro-8-methyl-1H-quinazolinedione;

7-[3-(1-Amino-2-hydroxyethyl)pyrrolidin-1-yl]-1-cyclopropyl-5-difluoromethyl-6-fluoro-8-methyl-1H-quinazolinedione;

7-[3-(1-Amino-3,3,3-trifluoro-2-trifluoromethylpropyl)pyrrolidin-1-yl]-1-cyclopropyl-5-difluoromethyl-6-fluoro-8-methyl-1H-quinazolinedione;

7-(4-Aminomethyl-4,5,6,7-tetrahydrobenzo[b]thiophen-2-yl)-1-cyclopropyl-5-difluoromethyl-6-fluoro-8-methyl-1H-quinazolinedione;

1-Cyclopropyl-5-difluoromethyl-6-fluoro-7-(4-hydroxymethyl-4,5,6,7-tetrahydro-benzo[b]thiophen-2-yl)-8-methyl-1H-quinazolinedione;

7-[4-(1-Aminoethyl)-4,5,6,7-tetrahydrobenzo[b]thiophen-2-yl]-1-cyclopropyl-5-difluoromethyl-6-fluoro-8-methyl-1H-quinazolinedione;

7-[4-(2-Amino-1-hydroxyethyl)-4,5,6,7-tetrahydrobenzo[b]thiophen-2-yl]-1-cyclopropyl-5-difluoromethyl-6-fluoro-8-methyl-1H-quinazolinedione;

7-[4-(1-Amino-2-hydroxyethyl)-4,5,6,7-tetrahydrobenzo[b]thiophen-2-yl]-1-cyclopropyl-5-difluoromethyl-6-fluoro-8-methyl-1H-quinazolinedione;

7-[4-(1-Amino-2,2,2-trifluoroethyl)-4,5,6,7-tetrahydrobenzo[b]thiophen-2-yl]-1-cyclopropyl-5-difluoromethyl-6-fluoro-8-methyl-1H-quinazolinedione;

7-(5-Aminomethyl-4,5,6,7-tetrahydrobenzo[b]thiophen-2-yl)-1-cyclopropyl-5-difluoromethyl-6-fluoro-8-methyl-1H-quinazolinedione;

1-Cyclopropyl-5-difluoromethyl-6-fluoro-7-(5-hydroxymethyl-4,5,6,7-tetrahydro-benzo[b]thiophen-2-yl)-8-methyl-1H-quinazolinedione;

7-[5-(1-Amino-2,2,2-trifluoroethyl)-4,5,6,7-tetrahydrobenzo[b]thiophen-2-yl]-1-cyclopropyl-5-difluoromethyl-6-fluoro-8-methyl-1H-quinazolinedione;

7-[5-(1-Aminoethyl)-4,5,6,7-tetrahydrobenzo[b]thiophen-2-yl]-1-cyclopropyl-5-difluoromethyl-6-fluoro-8-methyl-1H-quinazolinedione;

7-[5-(1-Amino-2-hydroxyethyl)-4,5,6,7-tetrahydrobenzo[b]thiophen-2-yl]-1-cyclopropyl-5-difluoromethyl-6-fluoro-8-methyl-1H-quinazolinedione;

7-(3-Aminomethyl-4-trifluoromethylpyrrolidin-1-yl)-1-cyclopropyl-5-difluoromethyl-6-fluoro-8-methyl-1H-quinazolinedione;

7-[3-(1-Amino-2,2,3,3,3-pentafluoropropyl)pyrrolidin-1-yl]-1-cyclopropyl-5-difluoromethyl-6-fluoro-8-methyl-1H-quinazolinedione;

7-[3-(1-Amino-3,3,3-trifluoropropyl)pyrrolidin-1-yl]-1-cyclopropyl-5-difluoromethyl-6-fluoro-8-methyl-1H-quinazolinedione;

7-[3-(1-Amino-3,3,3-trifluoro-2-trifluoromethylpropyl)pyrrolidin-1-yl]-1-cyclopropyl-5-difluoromethyl-6-fluoro-8-methyl-1H-quinazolinedione

7-[4-(1-Aminoethyl)-3,3-difluoropyrrolidin-1-yl]-1-cyclopropyl-5-difluoromethyl-6-fluoro-8-methyl-1H-quinazolinedione;

7-(3-Amino-4-ethylpiperidin-1-yl)-1-cyclopropyl-5-difluoromethyl-6-fluoro-8-methyl-1H-quinazolinedione;

7-[3-(1-Amino-2-trifluoromethoxyethyl)pyrrolidin-1-yl]-1-cyclopropyl-5-difluoromethyl-6-fluoro-8-methyl-1H-quinazolinedione;

7-(3-Aminomethyl-4-trifluoromethoxypyrrolidin-1-yl)-1-cyclopropyl-5-difluoromethyl-6-fluoro-8-methyl-1H-quinazolinedione;

7-[3-Aminomethyl-4-(2,2,2-trifluoroethyl)pyrrolidin-1-yl]-1-cyclopropyl-5-difluoro-methyl-6-fluoro-8-methyl-1H-quinazolinedione

7-[3-(1-Amino-2-difluoromethyl-3,3-difluoropropyl)pyrrolidin-1-yl]-1-cyclopropyl-5-difluoromethyl-6-fluoro-8-methyl-1H-quinazolinedione;

7-[3-(1-Amino-3-fluoro-2-fluoromethylpropyl)pyrrolidin-1-yl]-1-cyclopropyl-5-difluoromethyl-6-fluoro-8-methyl-1H-quinazolinedione;

7-[3-(1-Amino-3,3-difluoropropyl)pyrrolidin-1-yl]-1-cyclopropyl-5-difluoromethyl-6-fluoro-8-methyl-1H-quinazolinedione;

1-Cyclopropyl-7-(3,3-difluoro-4-hydroxymethylpyrrolidin-1-yl)-5-difluoromethyl-6-fluoro-8-methyl-1H-quinazolinedione;

1-Cyclopropyl-5-difluoromethyl-7-[7-(1,2-dihydroxyethyl)-5-azaspiro[2.4]hept-5-yl]-6-fluoro-8-methyl-1H-quinazolinedione;

1-Cyclopropyl-7-(5,5-difluoro-4-hydroxymethyl-4,5,6,7-tetrahydrobenzo[b]thiophen-2-yl)-5-difluoromethyl-6-fluoro-8-methyl-1H-quinazolinedione;

7-[3-(1-Amino-2,2,2-trifluoro-1-trifluoromethylethyl)pyrrolidin-1-yl]-1-cyclopropyl-5-difluoromethyl-6-fluoro-8-methyl-1H-quinazolinedione;

7-[3-Aminomethyl-4-(3,3,3-trifluoro-2-trifluoromethylpropyl)pyrrolidin-1-yl]-1-cyclopropyl-5-difluoromethyl-6-fluoro-8-methyl-1H-quinazolinedione;

7-[3-(1-Amino-4,4,4-trifluoro-3-trifluoromethylbut-2-enyl)pyrrolidin-1-yl]-1-cyclopropyl-5-difluoromethyl-6-fluoro-8-methyl-1H-quinazolinedione;

7-[3-(1-Amino-4,4,4-trifluoro-3-trifluoromethylbutyl)pyrrolidin-1-yl-1-cyclopropyl-5-difluoromethyl-6-fluoro-8-methyl-1H-quinazolinedione;

7-[3-(1-Amino-2-hydroxyethyl)-4-fluoropyrrolidin-1-yl]-1-cyclopropyl-5-difluoromethyl-6-fluoro-8-methyl-1H-quinazolinedione;

7-[3-(1-Amino-2,2,2-trifluoroethyl)-4-fluoropyrrolidin-1-yl]-1-cyclopropyl-5-difluoromethyl-6-fluoro-8-methyl-1H-quinazolinedione;

7-(3-Aminomethyl-4-difluoromethoxypyrrolidin-1-yl)-1-cyclopropyl-5-difluoromethyl-6-fluoro-8-methyl-1H-quinazolinedione;

7-(4-Aminomethyl-5,5-difluoro-4,5,6,7-tetrahydrobenzo[b]thiophen-2-yl)-1-cyclopropyl-5-difluoromethyl-6-fluoro-8-methyl-1H-quinazolinedione;

7-(4-Amino-5,5-difluorooctahydrocyclohepta[c]pyrrol-2-yl)-1-cyclopropyl-5-difluoromethyl-6-fluoro-8-methyl-1H-quinazolinedione;

1-Cyclopropyl-7-(5,5-difluoro-4-hydroxyoctahydrocyclohepta[c]pyrrol-2-yl)-5-difluoromethyl-6-fluoro-8-methyl-1H-quinazolinedione;

7-[5-(1-Aminoethyl)thiophen-3-yl]-1-cyclopropyl-5-difluoromethyl-6-fluoro-8-methyl-1H-quinazolinedione;

7-(5-Aminomethylthiophen-3-yl)-1-cyclopropyl-5-difluoromethyl-6-fluoro-8-methyl-1H-quinazolinedione;

7-(4-Amino-5,5-difluoro-4,5,6,7-tetrahydrobenzo[b]thiophen-2-yl)-1-cyclopropyl-5-difluoromethyl-6-fluoro-8-methyl-1H-quinazolinedione;

1-Cyclopropyl-7-(5,5-difluoro-4-hydroxy-4,5,6,7-tetrahydrobenzo[b]thiophen-2-yl)-5-difluoromethyl-6-fluoro-8-methyl-1H-quinazolinedione;

7-(4-Amino-5,5-difluorooctahydroisoindol-2-yl)-1-cyclopropyl-5-difluoromethyl-6-fluoro-8-methoxy-1H-quinazolinedione;

7-(4-Amino-5,5-difluorohexahydrocyclopenta[c]pyrrol-2-yl)-1-cyclopropyl-5-difluoro-methyl-6-fluoro-8-methoxy-1H-quinazolinedione;

1-Cyclopropyl-7-(5,5-difluoro-4-hydroxyhexahydrocyclopenta[c]pyrrol-2-yl)-5-difluoro-methyl-6-fluoro-8-methoxy-1H-quinazolinedione;

7-[3-(1-Amino-2-hydroxyethyl)pyrrolidin-1-yl]-1-cyclopropyl-5-difluoromethyl-6-fluoro-8-methoxy-1H-quinazolinedione;

1-Cyclopropyl-7-(5,5-difluoro-4-hydroxyoctahydroisoindol-2-yl)-5-difluoromethyl-6-fluoro-8-methoxy-1H-quinazolinedione;

7-[3-(1-Amino-2-hydroxyethyl)pyrrolidin-1-yl]-1-cyclopropyl-5-difluoromethyl-6-fluoro-8-methoxy-1H-quinazolinedione;

7-[3-(1-Amino-3,3,3-trifluoro-2-trifluoromethylpropyl)pyrrolidin-1-yl]-1-cyclopropyl-5-difluoromethyl-6-fluoro-8-methoxy-1H-quinazolinedione;

7-(4-Aminomethyl-4,5,6,7-tetrahydrobenzo[b]thiophen-2-yl)-1-cyclopropyl-5-difluoromethyl-6-fluoro-8-methoxy-1H-quinazolinedione;

1-Cyclopropyl-5-difluoromethyl-6-fluoro-7-(4-hydroxymethyl-4,5,6,7-tetrahydro-benzo[b]thiophen-2-yl)-8-methooxy-1H-quinazolinedione;

7-[4-(1-Aminoethyl)-4,5,6,7-tetrahydrobenzo[b]thiophen-2-yl]-1-cyclopropyl-5-difluoromethyl-6-fluoro-8-methoxy-1H-quinazolinedione;

7-[4-(2-Amino-1-hydroxyethyl)-4,5,6,7-tetrahydrobenzo[b]thiophen-2-yl]-1-cyclopropyl-5-difluoromethyl-6-fluoro-8-methoxy-1H-quinazolinedione;

7-[4-(1-Amino-2-hydroxyethyl)-4,5,6,7-tetrahydrobenzo[b]thiophen-2-yl]-1-cyclopropyl-5-difluoromethyl-6-fluoro-8-methoxy-1H-quinazolinedione;

7-[4-(1-Amino-2,2,2-trifluoroethyl)-4,5,6,7-tetrahydrobenzo[b]thiophen-2-yl]-1-cyclopropyl-5-difluoromethyl-6-fluoro-8-methoxy-1H-quinazolinedione

7-(5-Aminomethyl-4,5,6,7-tetrahydrobenzo[b]thiophen-2-yl)-1-cyclopropyl-5-difluoromethyl-6-fluoro-8-methoxy-1H-quinazolinedione;

1-Cyclopropyl-5-difluoromethyl-6-fluoro-7-(5-hydroxymethyl-4,5,6,7-tetrahydro-benzo[b]thiophen-2-yl)-8-methoxy-1H-quinazolinedione;

7-[5-(1-Amino-2,2,2-trifluoroethyl)-4,5,6,7-tetrahydrobenzo[b]thiophen-2-yl]-1-cyclopropyl-5-difluoromethyl-6-fluoro-8-methoxy-1H-quinazolinedione

7-[5-(1-Aminoethyl)-4,5,6,7-tetrahydrobenzo[b]thiophen-2-yl]-1-cyclopropyl-5-difluoromethyl-6-fluoro-8-methoxy-1H-quinazolinedione;

7-[5-(1-Amino-2-hydroxyethyl)-4,5,6,7-tetrahydrobenzo[b]thiophen-2-yl]-1-cyclopropyl-5-difluoromethyl-6-fluoro-8-methoxy-1H-quinazolinedione;

7-[5-(2-Amino-1-hydroxyethyl)-4,5,6,7-tetrahydrobenzo[b]thiophen-2-yl]-1-cyclopropyl-5-difluoromethyl-6-fluoro-8-methoxy-1H-quinazolinedione;

7-(3-Aminomethyl-4-trifluoromethylpyrrolidin-1-yl)-1-cyclopropyl-5-difluoromethyl-6-fluoro-8-methoxy-1H-quinazolinedione;

7-[3-(1-Amino-2,2,3,3,3-pentafluoropropyl)pyrrolidin-1-yl]-1-cyclopropyl-5-difluoromethyl-6-fluoro-8-methoxy-1H-quinazolinedione;

7-[4-(1-Aminoethyl)-3,3-difluoropyrrolidin-1-yl]-1-cyclopropyl-5-difluoromethyl-6-fluoro-8-methoxy-1H-quinazolinedione;

7-(3-Amino-4-ethylpiperidin-1-yl)-1-cyclopropyl-5-difluoromethyl-6-fluoro-8-methoxy-1H-quinazolinedione;

7-[3-(1-Amino-2-trifluoromethoxyethyl)pyrrolidin-1-yl]-1-cyclopropyl-5-difluoromethyl-6-fluoro-8-methoxy-1H-quinazolinedione;

7-(3-Aminomethyl-4-trifluoromethoxypyrrolidin-1-yl)-1-cyclopropyl-5-difluoromethyl-6-fluoro-8-methoxy-1H-quinazolinedione;

7-[3-Aminomethyl-4-(2,2,2-trifluoroethyl)pyrrolidin-1-yl]-1-cyclopropyl-5-difluoro-methyl-6-fluoro-8-methoxy-1H-quinazolinedione;

7-[3-(1-Amino-2-difluoromethyl-3,3-difluoropropyl)pyrrolidin-1-yl]-1-cyclopropyl-5-difluoromethyl-6-fluoro-8-methoxy-1H-quinazolinedione;

7-[3-(1-Amino-3-fluoro-2-fluoromethylpropyl)pyrrolidin-1-yl]-1-cyclopropyl-5-difluoromethyl-6-fluoro-8-methoxy-1H-quinazolinedione;

7-[3-(1-Amino-3,3-difluoropropyl)pyrrolidin-1-yl]-1-cyclopropyl-5-difluoromethyl-6-fluoro-8-methoxy-1H-quinazolinedione;

1-Cyclopropyl-7-(3,3-difluoro-4-hydroxymethylpyrrolidin-1-yl)-5-difluoromethyl-6-fluoro-8-methoxy-1H-quinazolinedione;

1-Cyclopropyl-5-difluoromethyl-7-[7-(1,2-dihydroxyethyl)-5-azaspiro[2.4]hept-5-yl]-6-fluoro-8-methoxy-1H-quinazolinedione;

1-Cyclopropyl-7-(5,5-difluoro-4-hydroxymethyl-4,5,6,7-tetrahydrobenzo[b]thiophen-2-yl)-5-difluoromethyl-6-fluoro-8-methoxy-1H-quinazolinedione;

7-[3-(1-Amino-2,2,2-trifluoro-1-trifluoromethylethyl)pyrrolidin-1-yl]-1-cyclopropyl-5-difluoromethyl-6-fluoro-8-methoxy-1H-quinazolinedione;

7-[3-Aminomethyl-4-(3,3,3-trifluoro-2-trifluoromethylpropyl)pyrrolidin-1-yl]-1-cyclopropyl-5-difluoromethyl-6-fluoro-8-methoxy-1H-quinazolinedione;

7-[3-(1-Amino-4,4,4-trifluoro-3-trifluoromethylbut-2-enyl)pyrrolidin-1-yl]-1-cyclopropyl-5-difluoromethyl-6-fluoro-8-methoxy-1H-quinazolinedione;

7-[3-(1-Amino-4,4,4-trifluoro-3-trifluoromethylbutyl)pyrrolidin-1-yl]-]-cyclopropyl-5-difluoromethyl-6-fluoro-8-methoxy-1H-quinazolinedione;

7-[3-(1-Amino-2-hydroxyethyl)-4-fluoropyrrolidin-1-yl]-1-cyclopropyl-5-difluoromethyl-6-fluoro-8-methoxy-1H-quinazolinedione;

7-[3-(1-Amino-2,2,2-trifluoroethyl)-4-fluoropyrrolidin-1-yl]-1-cyclopropyl-5-difluoromethyl-6-fluoro-8-methoxy-1H-quinazolinedione;

7-(3-Aminomethyl-4-difluoromethoxypyrrolidin-1-yl)-1-cyclopropyl-5-difluoromethyl-6-fluoro-8-methoxy-1H-quinazolinedione;

7-(4-Aminomethyl-5,5-difluoro-4,5,6,7-tetrahydrobenzo[b]thiophen-2-yl)-1-cyclopropyl-5-difluoromethyl-6-fluoro-8-methoxy-1H-quinazolinedione;

7-(4-Amino-5,5-difluorooctahydrocyclohepta[c]pyrrol-2-yl)-1-cyclopropyl-5-difluoromethyl-6-fluoro-8-methoxy-1H-quinazolinedione;

1-Cyclopropyl-7-(5,5-difluoro-4-hydroxyoctahydrocyclohepta[c]pyrrol-2-yl)-5-difluoromethyl-6-fluoro-8-methoxy-1H-quinazolinedione;

7-[5-(1-Aminoethyl)thiophen-3-yl]-1-cyclopropyl-5-difluoromethyl-6-fluoro-8-methoxy-1H-quinazolinedione;

7-(5-Aminomethylthiophen-3-yl)-1-cyclopropyl-5-difluoromethyl-6-fluoro-8-methoxy-1H-quinazolinedione;

7-(4-Amino-5,5-difluoro-4,5,6,7-tetrahydrobenzo[b]thiophen-2-yl)-1-cyclopropyl-5-difluoromethyl-6-fluoro-8-methoxy-1H-quinazolinedione;

1-Cyclopropyl-7-(5,5-difluoro-4-hydroxy-4,5,6,7-tetrahydrobenzo[b]thiophen-2-yl)-5,8-dimethyl-6-fluoro-1H-quinazolinedione;

7-(4-Amino-5,5-difluorooctahydroisoindol-2-yl)-1-cyclopropyl-5,8-dimethyl-6-fluoro-1H-quinazolinedione;

7-(4-Amino-5,5-difluorohexahydrocyclopenta[c]pyrrol-2-yl)-1-cyclopropyl-5,8-dimethyl-6-fluoro-1H-quinazolinedione;

7-(5,5-Difluoro-4-hydroxyoctahydroisoindol-2-yl)-1-cyclopropyl-5,8-dimethyl-6-fluoro-1H-quinazolinedione;

7-(5,5-Difluoro-4-hydroxyhexahydrocyclopenta[c]pyrrol-2-yl)-1-cyclopropyl-5,8-dimethyl-6-fluoro-1H-quinazolinedione;

7-[3-(1-Amino-2-hydroxyethyl)pyrrolidin-1-yl]-1-cyclopropyl-5-dimethyl-6-fluoro-1H-quinazolinedione;

7-[3-(1-Amino-3,3,3-trifluoro-2-trifluoromethylpropyl)pyrrolidin-1-yl]-1-cyclopropyl-5,8-dimethyl-6-fluoro-1H-quinazolinedione;

7-(4-Aminomethyl-4,5,6,7-tetrahydrobenzo[b]thiophen-2-yl)-1-cyclopropyl-5,8-dimethyl-6-fluoro-1H-quinazolinedione;

1-Cyclopropyl-5,8-dimethyl-6-fluoro-7-(4-hydroxymethyl-4,5,6,7-tetrahydro-benzo[b]thiophen-2-yl)-1H-quinazolinedione;

7-[4-(1-Aminoethyl)-4,5,6,7-tetrahydrobenzo[b]thiophen-2-yl]-1-cyclopropyl-5,8-dimethyl-6-fluoro-1H-quinazolinedione;

7-[4-(2-Amino-1-hydroxyethyl)-4,5,6,7-tetrahydrobenzo[b]thiophen-2-yl]-1-cyclopropyl-5,8-dimethyl-6-fluoro-1H-quinazolinedione;

7-[4-(1-Amino-2-hydroxyethyl)-4,5,6,7-tetrahydrobenzo[b]thiophen-2-yl]-1-cyclopropyl-5,8-dimethyl-6-fluoro-1H-quinazolinedione;

7-[4-(1-Amino-2,2,2-trifluoroethyl)-4,5,6,7-tetrahydrobenzo[b]thiophen-2-yl]-1-cyclopropyl-5,8-dimethyl-6-fluoro-1H-quinazolinedione;

7-(5-Aminomethyl-4,5,6,7-tetrahydrobenzo[b]thiophen-2-yl)-1-cyclopropyl-5,8-dimethyl-6-fluoro-1H-quinazolinedione;

1-Cyclopropyl-5,8-dimethyl-6-fluoro-7-(5-hydroxymethyl-4,5,6,7-tetrahydro-benzo[b]thiophen-2-yl)-1H-quinazolinedione;

7-[5-(1-Amino-2,2,2-trifluoroethyl)-4,5,6,7-tetrahydrobenzo[b]thiophen-2-yl]-1-cyclopropyl-5,8-methyl-6-fluoro-1H-quinazolinedione;

7-(3-Aminomethyl-4-trifluoromethylpyrrolidin-1-yl)-1-cyclopropyl-5,8-dimethyl-6-fluoro-1H-quinazolinedione;

7-[3-(1-Amino-2,2,3,3,3-pentafluoropropyl)pyrrolidin-1-yl]-1-cyclopropyl-5,8-dimethyl-6-fluoro-1H-quinazolinedione;

7-[3-(1-Amino-3,3,3-trifluoropropyl)pyrrolidin-1-yl]-1-cyclopropyl-5,8-dimethyl-6-fluoro-1H-quinazolinedione;

7-[3-(1-Amino-3,3,3-trifluoro-2-trifluoromethylpropyl)pyrrolidin-1-yl]-1-cyclopropyl-5,8-dimethyl-6-fluoro-1H-quinazolinedione;

7-(3-Aminomethyl-4-trifluoromethylpyrrolidin-1-yl)-1-cyclopropyl-5,8-dimethyl-6-fluoro-1H-quinazolinedione;

7-[3-(1-Amino-2,2,3,3,3-pentafluoropropyl)pyrrolidin-1-yl]-1-cyclopropyl-5,8-dimethyl-6-fluoro-1H-quinazolinedione;

7-[3-(1-Amino-3,3,3-trifluoropropyl)pyrrolidin-1-yl]-1-cyclopropyl-5,8-dimethyl-6-fluoro-1H-quinazolinedione;

7-[4-(1-Aminoethyl)-3,3-difluoropyrrolidin-1-yl]-1-cyclopropyl-5,8-dimethyl-6-fluoro-1H-quinazolinedione;

7-(3-Amino-4-ethylpiperidin-1-yl)-1-cyclopropyl-5,8-dimethyl-6-fluoro-8-1H-quinazolinedione;

7-[3-(1-Amino-2-trifluoromethoxyethyl)pyrrolidin-1-yl]-1-cyclopropyl-5,8-dimethyl-6-fluoro-1H-quinazolinedione;

7-(3-Aminomethyl-4-trifluoromethoxypyrrolidin-1-yl)-1-cyclopropyl-5,8-dimethyl-6-fluoro-1H-quinazolinedione;

7-[3-Aminomethyl-4-(2,2,2-trifluoroethyl)pyrrolidin-1-yl]-1-cyclopropyl-5,8-dimethyl-6-fluoro-1H-quinazolinedione

7-[3-(1-Amino-2,2-difluoromethyl-3,3-difluoropropyl)pyrrolidin-1-yl]-1-cyclopropyl-5,8-dimethyl-6-fluoro-1H-quinazolinedione;

7-[3-(1-Amino-3-fluoro-2-fluoromethylpropyl)pyrrolidin-1-yl]-1-cyclopropyl-5,8-dimethyl-6-fluoro-1H-quinazolinedione;

7-[3-(1-Amino-3,3-difluoropropyl)pyrrolidin-1-yl]-1-cyclopropyl-5,8-dimethyl-6-fluoro-1H-quinazolinedione;

1-Cyclopropyl-7-(3,3-difluoro-4-hydroxymethylpyrrolidin-1-yl)-5,8-dimethyl-6-fluoro-1H-quinazolinedione;

1-Cyclopropyl-5,8-dimethyl-7-[7-(1,2-dihydroxyethyl)-5-azaspiro[2.4]hept-5-yl]-6-fluoro-1H-quinazolinedione;

1-Cyclopropyl-7-(5,5-difluoro-4-hydroxymethyl-4,5,6,7-tetrahydrobenzo[b]thiophen-2-yl)-5,8-dimethyl-6-fluoro-1H-quinazolinedione;

7-[3-(1-Amino-2,2,2-trifluoro-1,1,1-trifluoromethylethyl)pyrrolidin-1-yl]-1-cyclopropyl-5,8-dimethyl-6-fluoro-1H-quinazolinedione;

7-[3-Aminomethyl-4-(3,3,3-trifluoro-2,2,2-trifluoromethylpropyl)pyrrolidin-1-yl]-1-cyclopropyl-5,8-dimethyl-6-fluoro-1H-quinazolinedione;

7-[3-(1-Amino-4,4,4-trifluoro-3,3,-trifluoromethylbut-2-enyl)pyrrolidin-1-yl-1-cyclopropyl-5,8-dimethyl-6-fluoro-1H-quinazolinedione;

7-[3-(1-Amino-4,4,4-trifluoro-3,3,3-trifluoromethylbutyl)pyrrolidin-1-yl]-1-cyclopropyl-5,8-dimethyl-6-fluoro-1H-quinazolinedione;

7-[3-(1-Amino-2-hydroxyethyl)-4-fluoropyrrolidin-1-yl]-1-cyclopropyl-5,8-dimethyl-6-fluoro-1H-quinazolinedione;

7-[3-(1-Amino-2,2,2-trifluoroethyl)-4-fluoropyrrolidin-1-yl]-1-cyclopropyl-5,8-dimethyl-6-fluoro-1H-quinazolinedione;

7-(3-Aminomethyl-4,4-difluoromethoxypyrrolidin-1-yl)-1-cyclopropyl-5,8-dimethyl-6-fluoro-1H-quinazolinedione;

7-(4-Aminomethyl-5,5-difluoro-4,5,6,7-tetrahydrobenzo[b]thiophen-2-yl)-1-cyclopropyl-5,8-dimethyl-6-fluoro-1H-quinazolinedione;

7-(4-Amino-5,5-difluorooctahydrocyclohepta[c]pyrrol-2-yl)-1-cyclopropyl-5,8-dimethyl-6-fluoro-1H-quinazolinedione;

1-Cyclopropyl-7-(5,5-difluoro-4-hydroxyoctahydrocyclohepta[c]pyrrol-2-yl)-5,8-dimethyl-6-fluoro-1H-quinazolinedione;

7-[5-(1-Aminoethyl)thiophen-3-yl]-1-cyclopropyl-5,8-dimethyl-6-fluoro-1H-quinazolinedione;

7-(5-Aminomethylthiophen-3-yl)-1-cyclopropyl-5,8-dimethyl-6-fluoro-1H-quinazolinedione;

7-(4-Amino-5,5-difluoro-4,5,6,7-tetrahydrobenzo[b]thiophen-2-yl)-1-cyclopropyl-5,8-dimethyl-6-fluoro-1H-quinazolinedione;

1-Cyclopropyl-7-(5,5-difluoro-4-hydroxy-4,5,6,7-tetrahydrobenzo[b]thiophen-2-yl)-5,8-dimethyl-6-fluoro-1H-quinazolinedione;

7-(4-Amino-5,5-difluorooctahydroisoindol-2-yl)-1-cyclopropyl-5-methyl-6-fluoro-8-methoxy-1H-quinazolinedione;

7-(4-Amino-5,5-difluorohexahydrocyclopenta[c]pyrrol-2-yl)-1-cyclopropyl-5-methyl-6-fluoro-8-methoxy-1H-quinazolinedione;

7-(5,5-Difluoro-4-hydroxyoctahydroisoindol-2-yl)-1-cyclopropyl-5-methyl-6-fluoro-8-methoxy-1H-quinazolinedione;

7-(5,5-Difluoro-4-hydroxyhexahydrocyclopenta[c]pyrrol-2-yl)-1-cyclopropyl-5-methyl-6-fluoro-8-methoxy-1H-quinazolinedione;

7-[3-(1-Amino-2-hydroxyethyl)pyrrolidin-1-yl]-1-cyclopropyl-5-methyl-6-fluoro-8-methoxy-1H-quinazolinedione;

7-[3-(1-Amino-3,3,3-trifluoro-2,2,2-trifluoromethylpropyl)pyrrolidin-1-yl]-1-cyclopropyl-5-methyl-6-fluoro-8-methoxy-1H-quinazolinedione;

7-(4-Aminomethyl-4,5,6,7-tetrahydrobenzo[b]thiophen-2-yl)-1-cyclopropyl-5-methyl-6-fluoro-8-methoxy-1H-quinazolinedione;

1-Cyclopropyl-5-methyl-6-fluoro-7-(4-hydroxymethyl-4,5,6,7-tetrahydro-benzo[b]thiophen-2-yl)-8-methoxy-1H-quinazolinedione;

7-[4-(1-Aminoethyl)-4,5,6,7-tetrahydrobenzo[b]thiophen-2-yl]-1-cyclopropyl-5-methyl-6-fluoro-8-methoxy-1H-quinazolinedione;

7-[4-(2-Amino-1-hydroxyethyl)-4,5,6,7-tetrahydrobenzo[b]thiophen-2-yl]-1-cyclopropyl-5-methyl-6-fluoro-8-methoxy-1H-quinazolinedione;

7-[4-(1-Amino-2-hydroxyethyl)-4,5,6,7-tetrahydrobenzo[b]thiophen-2-yl]-1-cyclopropyl-5-methyl-6-fluoro-8-methoxy-1H-quinazolinedione;

7-[4-(1-Amino-2,2,2-trifluoroethyl)-4,5,6,7-tetrahydrobenzo[b]thiophen-2-yl]-1-cyclopropyl-5-methyl-6-fluoro-8-methxoy-1H-quinazolinedione;

7-(5-Aminomethyl-4,5,6,7-tetrahydrobenzo[b]thiophen-2-yl)-1-cyclopropyl-5-methyl-6-fluoro-8-methoxy-1H-quinazolinedione;

1-Cyclopropyl-5-methyl-6-fluoro-7-(5-hydroxymethyl-4,5,6,7-tetrahydro-benzo[b]thiophen-2-yl)-8-methoxy-1H-quinazolinedione;

7-[5-(1-Amino-2,2,2-trifluoroethyl)-4,5,6,7-tetrahydrobenzo[b]thiophen-2-yl]-1-cyclopropyl-5-methyl-6-fluoro-8-methoxy-1H-quinazolinedione;

7-(3-Aminomethyl-4-trifluoromethylpyrrolidin-1-yl)-1-cyclopropyl-5-methyl-6-fluoro-8-methoxy-1H-quinazolinedione;

7-[3-(1-Amino-2,2,3,3,3-pentafluoropropyl)pyrrolidin-1-yl]-1-cyclopropyl-5-methyl-6-fluoro-8-methoxy-1H-quinazolinedione;

7-[3-(1-Amino-3,3,3-trifluoropropyl)pyrrolidin-1-yl]-1-cyclopropyl-5-methyl-6-fluoro-8-methoxy-] H-qui nazolinedione;

7-[3-(1-Amino-3,3,3-trifluoro-2,2,2-trifluoromethylpropyl)pyrrolidin-1-yl]-1-cyclopropyl-5-methyl-6-fluoro-8-methoxy-1H-quinazolinedione;

7-(3-Aminomethyl-4,4,4-trifluoromethylpyrrolidin-1-yl)-1-cyclopropyl-5-methyl-6-fluoro-8-methoxy-1H-quinazolinedione;

7-[3-(1-Amino-2,2,3,3,3-pentafluoropropyl)pyrrolidin-1-yl]-1-cyclopropyl-5-methyl-6-fluoro-8-methoxy-1H-quinazolinedione;

7-[3-(1-Amino-3,3,3-trifluoropropyl)pyrrolidin-1-yl]-1-cyclopropyl-5-methyl-6-fluoro-8-methoxy-1H-quinazolinedione;

7-(3-Aminomethyl-4-trifluoromethylpyrrolidin-1-yl)-1-cyclopropyl-5-methyl-6-fluoro-8-methoxy-1H-quinazolinedione;

7-[3-(1-Amino-2,2,3,3,3-pentafluoropropyl)pyrrolidin-1-yl]-1-cyclopropyl-5-methyl-6-fluoro-8-methoxy-1H-quinazolinedione;

7-[3-(1-Amino-3,3,3-trifluoropropyl)pyrrolidin-1-yl]-1-cyclopropyl-5-methyl-6-fluoro-8-methoxy-1H-quinazolinedione;

7-[4-(1-Aminoethyl)-3,3-difluoropyrrolidin-1-yl]-1-cyclopropyl-5-methyl-6-fluoro-8-methoxy-1H-quinazolinedione;

7-(3-Amino-4-ethylpiperidin-1-yl)-1-cyclopropyl-5-methyl-6-fluoro-8-methoxy-1H-quinazolinedione;

7-[3-(1-Amino-2-trifluoromethoxyethyl)pyrrolidin-1-yl]-1-cyclopropyl-5-methyl-6-fluoro-8-methoxy-1H-quinazolinedione;

7-(3-Aminomethyl-4,4,4-trifluoromethoxypyrrolidin-1-yl)-1-cyclopropyl-5-methyl-6-fluoro-8-methoxy-1H-quinazolinedione;

7-[3-Aminomethyl-4-(2,2,2-trifluoroethyl)pyrrolidin-1-yl]-1-cyclopropyl-5-methyl-6-fluoro-8-methoxy-1H-quinazolinedione;

7-[3-(1-Amino-2,2-difluoromethyl-3,3-difluoropropyl)pyrrolidin-1-yl]-1-cyclopropyl-5-methyl-6-fluoro-8-methoxy-1H-quinazolinedione;

7-[3-(1-Amino-3-fluoro-2-fluoromethylpropyl)pyrrolidin-1-yl]-1-cyclopropyl-5-methyl-6-fluoro-8-methoxy-1H-quinazolinedione;

7-[3-(1-Amino-3,3-difluoropropyl)pyrrolidin-1-yl]-1-cyclopropyl-5-methyl-6-fluoro-8-methoxy-1H-quinazolinedione;

1-Cyclopropyl-7-(3,3-difluoro-4-hydroxymethylpyrrolidin-1-yl)-5-methyl-6-fluoro-8-methoxy-1H-quinazolinedione;

1-Cyclopropyl-5-methyl-7-[7-(1,2-dihydroxyethyl)-5-azaspiro[2.4]hept-5-yl]-6-fluoro-8-methoxy-1H-quinazolinedione;

1-Cyclopropyl-7-(5,5-difluoro-4-hydroxymethyl-4,5,6,7-tetrahydrobenzo[b]thiophen-2-yl)-5-methyl-6-fluoro-8-methoxy-1H-quinazolinedione;

7-[3-(1-Amino-2,2,2-trifluoro-1,1,1-trifluoromethylethyl)pyrrolidin-1-yl]-1-cyclopropyl-5-methyl-6-fluoro-8-methoxy-1H-quinazolinedione;

7-[3-Aminomethyl-4-(3,3,3-trifluoro-2-trifluoromethylpropyl)pyrrolidin-1-yl]-1-cyclopropyl-5-methyl-6-fluoro-8-methoxy-1H-quinazolinedione;

7-[3-(1-Amino-4,4,4-trifluoro-3,3,3-trifluoromethylbut-2-enyl)pyrrolidin-1-yl-1-cyclopropyl-5-methyl-6-fluoro-8-methoxy-1H-quinazolinedione;

7-[3-(1-Amino-4,4,4-trifluoro-3,3,3-trifluoromethylbutyl)pyrrolidin-1-yl]-1-cyclopropyl-5-methyl-6-fluoro-8-methoxy-1H-quinazolinedione;

7-[3-(1-Amino-2-hydroxyethyl)-4-fluoropyrrolidin-1-yl]-1-cyclopropyl-5-methyl-6-fluoro-8-methoxy-1H-quinazolinedione;

7-[3-(1-Amino-2,2,2-trifluoroethyl)-4-fluoropyrrolidin-1-yl]-1-cyclopropyl-5-methyl-6-fluoro-8-methoxy-1H-quinazolinedione;

7-(3-Aminomethyl-4,4-difluoromethoxypyrrolidin-1-yl)-1-cyclopropyl-5-methyl-6-fluoro-8-methoxy-1H-quinazolinedione;

7-(4-Aminomethyl-5,5-difluoro-4,5,6,7-tetrahydrobenzo[b]thiophen-2-yl)-1-cyclopropyl-5-methyl-6-fluoro-8-methoxy-1H-quinazolinedione;

7-(4-Amino-5,5-difluorooctahydrocyclohepta[c]pyrrol-2-yl)-1-cyclopropyl-5-methyl-6-fluoro-8-methoxy-1H-quinazolinedione;

1-Cyclopropyl-7-(5,5-difluoro-4-hydroxyoctahydrocyclohepta[c]pyrrol-2-yl)-5-methyl-6-fluoro-8-methoxy-1H-quinazolinedione;

7-[5-(1-Aminoethyl)thiophen-3-yl]-1-cyclopropyl-5-methyl-6-fluoro-8-methoxy-1H-quinazolinedione;

7-(5-Aminomethylthiophen-3-yl)-1-cyclopropyl-5-methyl-6-fluoro-8-methoxy-1H-quinazolinedione;

7-(4-Amino-5,5-difluoro-4,5,6,7-tetrahydrobenzo[b]thiophen-2-yl)-1-cyclopropyl-5-methyl-6-fluoro-8-methoxy-1H-quinazolinedione;

1-Cyclopropyl-7-(5,5-difluoro-4-hydroxy-4,5,6,7-tetrahydrobenzo[b]thiophen-2-yl)-5-methyl-6-fluoro-8-methoxy-1H-quinazolinedione;

5-Amino-7-(4-amino-5,5-difluorooctahydroisoindol-2-yl)-1-cyclopropyl-6-fluoro-8-methyl-1H-quinazolinedione;

5-Amino-7-(4-amino-5,5-difluorohexahydrocyclopenta[c]pyrrol-2-yl)-1-cyclopropyl-6-fluoro-8-methyl-1H-quinazolinedione;

5-Amino-7-(5,5-difluoro-4-hydroxyoctahydroisoindol-2-yl)-1-cyclopropyl-6-fluoro-8-methyl-1H-quinazolinedione;

5-Amino-7-(5,5-difluoro-4-hydroxyhexahydrocyclopenta[c]pyrrol-2-yl)-1-cyclopropyl-6-fluoro-8-methyl-1H-quinazolinedione;

5-Amino-7-[3-(1-amino-2-hydroxyethyl)pyrrolidin-1-yl]-1-cyclopropyl-6-fluoro-8-methyl-1H-quinazolinedione;

5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazolinedione;

5-Amino-7-(4-aminomethyl-4,5,6,7-tetrahydrobenzo[b]thiophen-2-yl)-1-cyclopropyl-6-fluoro-8-methyl-1H-quinazolinedione;

5-Amino-1-cyclopropyl-6-fluoro-7-(4-hydroxymethyl-4,5,6,7-tetrahydrobenzo[b]thiophen-2-yl)-8-methyl-1H-quinazolinedione;

5-Amino-7-[4-(1-aminoethyl)-4,5,6,7-tetrahydrobenzo[b]thiophen-2-yl]-1-cyclopropyl-6-fluoro-8-methyl-1H-quinazolinedione;

5-Amino-7-[4-(2-amino-1-hydroxyethyl)-4,5,6,7-tetrahydrobenzo[b]thiophen-2-yl]-1-cyclopropyl-6-fluoro-8-methyl-1H-quinazolinedione;

5-Amino-7-[4-(1-amino-2-hydroxyethyl)-4,5,6,7-tetrahydrobenzo[b]thiophen-2-yl]-1-cyclopropyl-6-fluoro-8-methyl-1H-quinazolinedione;

5-Amino-7-[4-(1-amino-2,2,2-trifluoroethyl)-4,5,6,7-tetrahydrobenzo[b]thiophen-2-yl]-1-cyclopropyl-6-fluoro-8-methyl-1H-quinazolinedione;

5-Amino-7-(5-aminomethyl-4,5,6,7-tetrahydrobenzo[b]thiophen-2-yl)-1-cyclopropyl-6-fluoro-8-methyl-1H-quinazolinedione;

5-Amino-1-cyclopropyl-6-fluoro-7-(5-hydroxymethyl-4,5,6,7-tetrahydrobenzo[b]thiophen-2-yl)-8-methyl-1H-quinazolinedione;

5-Amino-7-[5-(1-amino-2,2,2-trifluoroethyl)-4,5,6,7-tetrahydrobenzo[b]thiophen-2-yl]-1-cyclopropyl-6-fluoro-8-methyl-1H-quinazolinedione;

5-Amino-7-(3-aminomethyl-4-trifluoromethylpyrrolidin-1-yl)-1-cyclopropyl-6-fluoro-8-methyl-1H-quinazolinedione;

5-Amino-7-[3-(1-amino-2,2,3,3,3-pentafluoropropyl)pyrrolidin-1-yl]-1-cyclopropyl-6-fluoro-8-methyl-1H-quinazolinedione;

5-Amino-7-[3-(1-amino-3,3,3-trifluoropropyl)pyrrolidin-1-yl]-1-cyclopropyl-6-fluoro-8-methyl-1H-quinazolinedione;

5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazolinedione;

5-Amino-7-(3-aminomethyl-4-trifluoromethylpyrrolidin-1-yl)-1-cyclopropyl-6-fluoro-8-methyl-1H-quinazolinedione;

5-Amino-7-[3-(1-amino-2,2,3,3,3-pentafluoropropyl)pyrrolidin-1-yl]-1-cyclopropyl-6-fluoro-8-methyl-1H-quinazolinedione;

5-Amino-7-[3-(1-amino-3-trifluoropropyl)pyrrolidin-1-yl]-1-cyclopropyl-6-fluoro-8-methyl-1H-quinazolinedione;

5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazolinedione;

5-Amino-7-[4-(1-aminoethyl)-3,3-difluoropyrrolidin-1-yl]-1-cyclopropyl-6-fluoro-8-methyl-1H-quinazolinedione;

5-Amino-7-(3-amino-4-ethylpiperidin-1-yl)-1-cyclopropyl-6-fluoro-8-methyl-1H-quinazolinedione;

5-Amino-7-[3-(1-amino-2,2,2-trifluoromethoxyethyl)pyrrolidin-1-yl]-1-cyclopropyl-5-difluoromethyl-6-fluoro-8-methyl-1H-quinazolinedione;

5-Amino-7-(3-aminomethyl-4-trifluoromethoxypyrrolidin-1-yl)-1-cyclopropyl-6-fluoro-8-methyl-1H-quinazolinedione;

5-Amino-7-[3-aminomethyl-4-(2,2,2-trifluoroethyl)pyrrolidin-1-yl]-1-cyclopropyl-6-fluoro-8-methyl-1H-quinazolinedione;

5-Amino-7-[3-(1-amino-2-difluoromethyl-3,3-difluoropropyl)pyrrolidin-1-yl]-1-cyclopropyl-6-fluoro-8-methyl-1H-quinazolinedione;

5-Amino-7-[3-(1-amino-3-fluoro-2-fluoromethylpropyl)pyrrolidin-1-yl]-1-cyclopropyl-6-fluoro-8-methyl-1H-quinazolinedione;

5-Amino-7-[3-(1-amino-3,3-difluoropropyl)pyrrolidin-1-yl]-1-cyclopropyl-6-fluoro-8-methyl-1H-quinazolinedione;

5-Amino-1-cyclopropyl-7-(3,3-difluoro-4-hydroxymethylpyrrolidin-1-yl)-6-fluoro-8-methyl-1H-quinazolinedione;

5-Amino-1-cyclopropyl-7-[7-(1,2-dihydroxyethyl)-5-azaspiro[2.4]hept-5-yl]-6-fluoro-8-methyl-1H-quinazolinedione;

5-Amino-1-cyclopropyl-7-(5,5-difluoro-4-hydroxymethyl-4,5,6,7-tetrahydrobenzo[b]thiophen-2-yl)-6-fluoro-8-methyl-1H-quinazolinedione;

5-Amino-7-[3-(1-amino-2,2,2-trifluoro-1-trifluoromethylethyl)pyrrolidin-1-yl]-1-cyclopropyl-6-fluoro-8-methyl-1H-quinazolinedione;

5-Amino-7-[3-aminomethyl-4-(3,3,3-trifluoro-2-trifluoromethylpropyl)pyrrolidin-1-yl]-1-cyclopropyl-6-fluoro-8-methyl-1H-quinazolinedione;

5-Amino-7-[3-(1-amino-4,4,4-trifluoro-3-trifluoromethyl-but-2-enyl)pyrrolidin-1-yl]-1-cyclopropyl-6-fluoro-8-methyl-1H-quinazolinedione;

5-Amino-7-[3-(1-amino-4,4,4-trifluoro-3-trifluoromethylbutyl)pyrrolidin-1-yl]-1-cyclopropyl-6-fluoro-8-methyl-1H-quinazolinedione;

5-Amino-7-[3-(1-amino-2-hydroxyethyl)-4-fluoropyrrolidin-1-yl]-1-cyclopropyl-6-fluoro-8-methyl-1H-quinazolinedione;

5-Amino-7-[3-(1-amino-2,2,2-trifluoroethyl)-4-fluoropyrrolidin-1-yl]-1-cyclopropyl-6-fluoro-8-methyl-1H-quinazolinedione;

5-Amino-7-(3-aminomethyl-4-difluoromethoxypyrrolidin-1-yl)-1-cyclopropyl-6-fluoro-8-methyl-1H-quinazolinedione;

5-Amino-7-(4-aminomethyl-5,5-difluoro-4,5,6,7-tetrahydrobenzo[b]thiophen-2-yl)-1-cyclopropyl-6-fluoro-8-methyl-1H-quinazolinedione;

5-Amino-7-(4-amino-5,5-difluorooctahydrocyclohepta[c]pyrrol-2-yl)-1-cyclopropyl-6-fluoro-8-methyl-1H-quinazolinedione;

5-Amino-1-cyclopropyl-7-(5,5-difluoro-4-hydroxyoctahydrocyclohepta[c]pyrrol-2-yl)-6-fluoro-8-methyl-1H-quinazolinedione;

5-Amino-7-[5-(1-aminoethyl)thiophen-3-yl]-1-cyclopropyl-6-fluoro-8-methyl-1H-quinazolinedione;

5-Amino-7-(5-aminomethylthiophen-3-yl)-1-cyclopropyl-6-fluoro-8-methyl-1H-quinazolinedione;

5-Amino-7-(4-amino-5,5-difluoro-4,5,6,7-tetrahydrobenzo[b]thiophen-2-yl)-1-cyclopropyl-6-fluoro-8-methyl-1H-quinazolinedione;

5-Amino-1-cyclopropyl-7-(5,5-difluoro-4-hydroxy-4,5,6,7-tetrahydrobenzo[b]thiophen-2-yl)-6-fluoro-8-methyl-1H-quinazolinedione;

5-Amino-7-(4-amino-5,5-difluorooctahydroisoindol-2-yl)-1-cyclopropyl-6-fluoro-8-methoxy-1H-quinazolinedione;

5-Amino-7-(4-amino-5,5-difluorohexahydrocyclopenta[c]pyrrol-2-yl)-1-cyclopropyl-6-fluoro-8-methoxy-1H-quinazolinedione;

5-Amino-7-(5,5-difluoro-4-hydroxyoctahydroisoindol-2-yl)-1-cyclopropyl-6-fluoro-8-methoxy-1H-quinazolinedione;

5-Amino-7-(5,5-difluoro-4-hydroxyhexahydrocyclopenta[c]pyrrol-2-yl)-1-cyclopropyl-6-fluoro-8-methoxy-1H-quinazolinedione;

5-Amino-7-[3-(1-amino-2-hydroxyethyl)pyrrolidin-1-yl]-1-cyclopropyl-6-fluoro-8-methoxy-1H-quinazolinedione;

5-Amino-7-[3-(1-amino-3,3,3-trifluoro-2-trifluoromethylpropyl)pyrrolidin-1-yl]-1-cyclopropyl-6-fluoro-8-methoxy-1H-quinazolinedione;

5-Amino-7-(4-aminomethyl-4,5,6,7-tetrahydrobenzo[b]thiophen-2-yl)-1-cyclopropyl-6-fluoro-8-methoxy-1H-quinazolinedione;

5-Amino-1-cyclopropyl-6-fluoro-7-(4-hydroxymethyl-4,5,6,7-tetrahydrobenzo[b]thiophen-2-yl)-8-methoxy-1H-quinazolinedione;

5-Amino-7-[4-(1-aminoethyl)-4,5,6,7-tetrahydrobenzo[b]thiophen-2-yl]-1-cyclopropyl-6-fluoro-8-methoxy-1H-quinazolinedione;

5-Amino-7-[4-(2-amino-1-hydroxyethyl)-4,5,6,7-tetrahydrobenzo[b]thiophen-2-yl]-1-cyclopropyl-6-fluoro-8-methoxy-1H-quinazolinedione;

5-Amino-7-[4-(1-amino-2-hydroxyethyl)-4,5,6,7-tetrahydrobenzo[b]thiophen-2-yl]-1-cyclopropyl-6-fluoro-8-methoxy-1H-quinazolinedione;

5-Amino-7-[4-(1-amino-2,2,2-trifluoroethyl)-4,5,6,7-tetrahydrobenzo[b]thiophen-2-yl]-1-cyclopropyl-6-fluoro-8-methoxy-1H-quinazolinedione;

5-Amino-7-(5-aminomethyl-4,5,6,7-tetrahydrobenzo[b]thiophen-2-yl)-1-cyclopropyl-6-fluoro-8-methoxy-1H-quinazolinedione;

5-Amino-1-cyclopropyl-6-fluoro-7-(5-hydroxymethyl-4,5,6,7-tetrahydrobenzo[b]thiophen-2-yl)-8-methoxy-1H-quinazolinedione;

5-Amino-7-[5-(1-amino-2,2,2-trifluoroethyl)-4,5,6,7-tetrahydrobenzo[b]thiophen-2-yl]-1-cyclopropyl-6-fluoro-8-methoxy-1H-quinazolinedione;

5-Amino-7-(3-aminomethyl-4-trifluoromethylpyrrolidin-1-yl)-1-cyclopropyl-6-fluoro-8-methoxy-1H-quinazolinedione;

5-Amino-7-[3-(1-amino-2,2,3,3,3-pentafluoropropyl)pyrrolidin-1-yl]-1-cyclopropyl-6-fluoro-8-methoxy-1H-quinazolinedione;

5-Amino-7-[3-(1-amino-3,3,3-trifluoropropyl)pyrrolidin-1-yl]-1-cyclopropyl-6-fluoro-8-methoxy-1H-quinazolinedione;

5-Amino-7-[3-(1-amino-3,3,3-trifluoro-2-trifluoromethylpropyl)pyrrolidin-1-yl]-1-cyclopropyl-6-fluoro-8-methoxy-1H-quinazolinedione;

5-Amino-7-(3-aminomethyl-4-trifluoromethylpyrrolidin-1-yl)-1-cyclopropyl-6-fluoro-8-methoxy-1H-quinazolinedione;

5-Amino-7-[3-(1-amino-2,2,3,3,3-pentafluoropropyl)pyrrolidin-1-yl]-1-cyclopropyl-6-fluoro-8-methoxy-1H-quinazolinedione;

5-Amino-7-[3-(1-amino-3,3,3-trifluoropropyl)pyrrolidin-1-yl]-1-cyclopropyl-6-fluoro-8-methoxy-1H-quinazolinedione;

5-Amino-7-[3-(1-amino-3,3,3-trifluoro-2-trifluoromethylpropyl)pyrrolidin-1-yl]-1-cyclopropyl-6-fluoro-8-methoxy-1H-quinazolinedione;

5-Amino-7-[4-(1-aminoethyl)-3,3-difluoropyrrolidin-1-yl]-1-cyclopropyl-6-fluoro-8-methoxy-1H-quinazolinedione;

5-Amino-7-(3-amino-4-ethylpiperidin-1-yl)-1-cyclopropyl-6-fluoro-8-methoxy-1H-quinazolinedione;

5-Amino-7-[3-(1-amino-2-trifluoromethoxyethyl)pyrrolidin-1-yl]-1-cyclopropyl-5-difluoromethyl-6-fluoro-8-methoxy-1H-quinazolinedione;

5-Amino-7-(3-aminomethyl-4-trifluoromethoxypyrrolidin-1-yl)-1-cyclopropyl-6-fluoro-8-methoxy-1H-quinazolinedione;

5-Amino-7-[3-aminomethyl-4-(2,2,2-trifluoroethyl)pyrrolidin-1-yl]-1-cyclopropyl-6-fluoro-8-methoxy-1H-quinazolinedione;

5-Amino-7-[3-(1-amino-2-difluoromethyl-3,3-difluoropropyl)pyrrolidin-1-yl]-1-cyclopropyl-6-fluoro-8-methoxy-1H-quinazolinedione;

5-Amino-7-[3-(1-amino-3-fluoro-2-fluoromethylpropyl)pyrrolidin-1-yl]-1-cyclopropyl-6-fluoro-8-methoxy-1H-quinazolinedione;

5-Amino-7-[3-(1-amino-3,3-difluoropropyl)pyrrolidin-1-yl]-1-cyclopropyl-6-fluoro-8-methoxy-1H-quinazolinedione;

5-Amino-1-cyclopropyl-7-(3,3-difluoro-4-hydroxymethylpyrrolidin-1-yl)-6-fluoro-8-methoxy-1H-quinazolinedione;

5-Amino-1-cyclopropyl-7-[7-(1,2-dihydroxyethyl)-5-azaspiro[2.4]hept-5-yl]-6-fluoro-8-methoxy-1H-quinazolinedione;

5-Amino-1-cyclopropyl-7-(5,5-difluoro-4-hydroxymethyl-4,5,6,7-tetrahydrobenzo[b]thiophen-2-yl)-6-fluoro-8-methoxy-1H-quinazolinedione;

5-Amino-7-[3-(1-amino-2,2,2-trifluoro-1-trifluoromethylethyl)pyrrolidin-1-yl]-1-cyclopropyl-6-fluoro-8-methoxy-1H-quinazolinedione;

5-Amino-7-[3-aminomethyl-4-(3,3,3-trifluoro-2-trifluoromethylpropyl)pyrrolidin-1-yl]-1-cyclopropyl-6-fluoro-8-methoxy-1H-quinazolinedione;

5-Amino-7-[3-(1-amino-4,4,4-trifluoro-3-trifluoromethyl-but-2-enyl)pyrrolidin-1-yl]-1-cyclopropyl-6-fluoro-8-methoxy-1H-quinazolinedione;

5-Amino-7-[3-(1-amino-4,4,4-trifluoro-3-trifluoromethylbutyl)pyrrolidin-1-yl]-1-cyclopropyl-6-fluoro-8-methoxy-1H-quinazolinedione;

5-Amino-7-[3-(1-amino-2-hydroxyethyl)-4-fluoropyrrolidin-1-yl]-1-cyclopropyl-6-fluoro-8-methoxy-1H-quinazolinedione;

5-Amino-7-[3-(1-amino-2,2,2-trifluoroethyl)-4-fluoropyrrolidin-1-yl]-1-cyclopropyl-6-fluoro-8-methoxy-1H-quinazolinedione;

5-Amino-7-(3-aminomethyl-4-difluoromethoxypyrrolidin-1-yl)-1-cyclopropyl-6-fluoro-8-methoxy-1H-quinazolinedione;

5-Amino-7-(4-aminomethyl-5,5-difluoro-4,5,6,7-tetrahydrobenzo[b]thiophen-2-yl)-1-cyclopropyl-6-fluoro-8-methoxy-1H-quinazolinedione;

5-Amino-7-(4-amino-5,5-difluorooctahydrocyclohepta[c]pyrrol-2-yl)-1-cyclopropyl-6-fluoro-8-methoxy-1H-quinazolinedione;

5-Amino-1-cyclopropyl-7-(5,5-difluoro-4-hydroxyoctahydrocyclohepta[c]pyrrol-2-yl)-6-fluoro-8-methoxy-1H-quinazolinedione;

5-Amino-7-[5-(1-aminoethyl)thiophen-3-yl-1-cyclopropyl-6-fluoro-8-methoxy-1H-quinazolinedione;

5-Amino-7-(5-aminomethylthiophen-3-yl)-1-cyclopropyl-6-fluoro-8-methoxy-1H-quinazolinedione;

5-Amino-7-(4-amino-5,5-difluoro-4,5,6,7-tetrahydrobenzo[b]thiophen-2-yl)-1-cyclopropyl-6-fluoro-8-methoxy-1H-quinazolinedione;

5-Amino-1-cyclopropyl-7-(5,5-difluoro-4-hydroxy-4,5,6,7-tetrahydrobenzo[b]thiophen-2-yl)₆-fluoro-8-methoxy-1H-quinazolinedione;

7-(4-Amino-5,5-difluorooctahydroisoindol-2-yl)-1-cyclopropyl-6-fluoro-5-hydroxy-8-methyl-1H-quinazolinedione;

7-(4-Amino-5,5-difluorohexahydrocyclopenta[c]pyrrol-2-yl)-1-cyclopropyl-6-fluoro-5-hydroxy-8-methyl-1H-quinazolinedione;

7-(5,5-Difluoro-4-hydroxyoctahydroisoindol-2-yl)-1-cyclopropyl-6-fluoro-5-hydroxy-8-methyl-1H-quinazolinedione;

7-(5,5-Difluoro-4-hydroxyhexahydrocyclopenta[c]pyrrol-2-yl)-1-cyclopropyl-6-fluoro-5-hydroxy-8-methyl-1H-quinazolinedione;

7-[3-(1-Amino-2-hydroxyethyl)pyrrolidin-1-yl]-1-cyclopropyl-6-fluoro-5-hydroxy-8-methyl-1H-quinazolinedione;

7-[3-(1-Amino-3,3,3-trifluoro-2-trifluoromethylpropyl)pyrrolidin-1-yl]-1-cyclopropyl-6-fluoro-5-hydroxy-8-methyl-1H-quinazolinedione;

7-(4-Aminomethyl-4,5,6,7-tetrahydrobenzo[b]thiophen-2-yl)-1-cyclopropyl-6-fluoro-5-hydroxy-8-methyl-1H-quinazolinedione;

1-Cyclopropyl-6-fluoro-7-(4-hydroxymethyl-4,5,6,7-tetrahydro-benzo[b]thiophen-2-yl)-5-hydroxy-8-methyl-1H-quinazolinedione;

7-[4-(1-Aminoethyl)-4,5,6,7-tetrahydrobenzo[b]thiophen-2-yl]-1-cyclopropyl-6-fluoro-5-hydroxy-8-methyl-1H-quinazolinedione;

7-[4-(2-Amino-1-hydroxyethyl)-4,5,6,7-tetrahydrobenzo[b]thiophen-2-yl]-1-cyclopropyl-6-fluoro-5-hydroxy-8-methyl-1H-quinazolinedione;

7-[4-(1-Amino-2-hydroxyethyl)-4,5,6,7-tetrahydrobenzo[b]thiophen-2-yl]-1-cyclopropyl-6-fluoro-5-hydroxy-8-methyl-1H-quinazolinedione;

7-[4-(1-Amino-2,2,2-trifluoroethyl)-4,5,6,7-tetrahydrobenzo[b]thiophen-2-yli]-1-cyclopropyl-6-fluoro-5-hydroxy-8-methyl-1H-quinazolinedione;

7-(5-Aminomethyl-4,5,6,7-tetrahydrobenzo[b]thiophen-2-yl)-1-cyclopropyl-6-fluoro-5-hydroxy-8-methyl-1H-quinazolinedione;

1-Cyclopropyl-6-fluoro-7-(5-hydroxymethyl-4,5,6,7-tetrahydro-benzo[b]thiophen-2-yl)-5-hydroxy-8-methyl-1H-quinazolinedione;

7-[5-(1-Amino-2,2,2-trifluoroethyl)-4,5,6,7-tetrahydrobenzo[b]thiophen-2-yl]-1-cyclopropyl-6-fluoro-5-hydroxy-8-methyl-1H-quinazolinedione;

7-[5-(1-Aminoethyl)-4,5,6,7-tetrahydrobenzo[b]thiophen-2-yl]-1-cyclopropyl-6-fluoro-5-hydroxy-8-methyl-1H-quinazolinedione;

7-[5-(1-Amino-2-hydroxyethyl)-4,5,6,7-tetrahydrobenzo[b]thiophen-2-yl]-1-cyclopropyl-6-fluoro-5-hydroxy-8-methyl-1H-quinazolinedione;

7-(3-Aminomethyl-4-trifluoromethylpyrrolidin-1-yl)-1-cyclopropyl-6-fluoro-5-hydroxy-8-methyl-1H-quinazolinedione;

7-[3-(1-Amino-2,2,3,3,3-pentafluoropropyl)pyrrolidin-1-yl]-1-cyclopropy-6-fluoro-5-hydroxy-8-methyl-1H-quinazolinedione;

7-[3-(1-Amino-3,3,3-trifluoropropyl)pyrrolidin-1-yl]-1-cyclopropyl-6-fluoro-5-hydroxy-8-methyl-1H-quinazolinedione;

7-[3-(1-Amino-3,3,3-trifluoro-2-trifluoromethylpropyl)pyrrolidin-1-yl]-1-cyclopropyl-6-fluoro-5-hydroxy-8-methyl-1H-quinazolinedione;

7-[4-(1-Aminoethyl)-3,3-difluoropyrrolidin-1-yl]-1-cyclopropyl-6-fluoro-5-hydroxy-8-methyl-1H-quinazolinedione;

7-(3-Amino-4-ethylpiperidin-1-yl)-1-cyclopropyl-6-fluoro-5-hydroxy-8-methyl-1H-quinazolinedione;

7-[3-(1-Amino-2-trifluoromethoxyethyl)pyrrolidin-1-yl]-1-cyclopropyl-6-fluoro-5-hydroxy-8-methyl-1H-quinazolinedione;

7-(3-Aminomethyl-4-trifluoromethoxypyrrolidin-1-yl)-1-cyclopropyl-6-fluoro-5-hydroxy-8-methyl-1H-quinazolinedione;

7-[3-Aminomethyl-4-(2,2,2-trifluoroethyl)pyrrolidin-1-yl]-1-cyclopropyl-6-fluoro-5-hydroxy-8-methyl-1H-quinazolinedione;

7-[3-(1-Amino-2-difluoromethyl-3,3-difluoropropyl)pyrrolidin-1-yl]-1-cyclopropyl-6-fluoro-5-hydroxy-8-methyl-1H-quinazolinedione;

7-[3-(1-Amino-3-fluoro-2-fluoromethylpropyl)pyrrolidin-1-yl]-1-cyclopropyl-6-fluoro-5-hydroxy-8-methyl-1H-quinazolinedione;

7-[3-(1-Amino-3,3-difluoropropyl)pyrrolidin-1-yl]-1-cyclopropyl-6-fluoro-5-hydroxy-8-methyl-1H-quinazolinedione;

1-Cyclopropyl-7-(3,3-difluoro-4-hydroxymethylpyrrolidin-1-yl)-6-fluoro-5-hydroxy-8-methyl-1H-quinazolinedione;

1-Cyclopropyl-7-[7-(1,2-dihydroxyethyl)-5-azaspiro[2.4]hept-5-yl]-6-fluoro-5-hydroxy-8-methyl-1H-quinazolinedione;

1-Cyclopropyl-7-(5,5-difluoro-4-hydroxymethyl-4,5,6,7-tetrahydrobenzo[b]thiophen-2-yl)-6-fluoro-5-hydroxy-8-methyl-1H-quinazolinedione;

7-[3-(1-Amino-2,2,2-trifluoro-1-trifluoromethylethyl)pyrrolidin-1-yl]-1-cyclopropyl-6-fluoro-5-hydroxy-8-methyl-1H-quinazolinedione;

7-[3-Aminomethyl-4-(3,3,3-trifluoro-2-trifluoromethylpropyl)pyrrolidin-1-yl]-1-cyclopropyl-6-fluoro-5-hydroxy-8-methyl-1H-quinazolinedione;

7-[3-(1-Amino-4,4,4-trifluoro-3-trifluoromethylbut-2-enyl)pyrrolidin-1-yl]-1-cyclopropyl-6-fluoro-5-hydroxy-8-methyl-1H-quinazolinedione;

7-[3-(1-Amino-4,4,4-trifluoro-3-trifluoromethylbutyl)pyrrolidin-1-yl]-1-cyclopropyl-6-fluoro-5-hydroxy-8-methyl-1H-quinazolinedione;

7-[3-(1-Amino-2-hydroxyethyl)-4-fluoropyrrolidin-1-yl]-1-cyclopropyl-6-fluoro-5-hydroxy-8-methyl-1H-quinazolinedione;

7-[3-(1-Amino-2,2,2-trifluoroethyl)-4-fluoropyrrolidin-1-yl]-1-cyclopropyl-6-fluoro-5-hydroxy-8-methyl-1H-quinazolinedione;

7-(3-Aminomethyl-4-difluoromethoxypyrrolidin-1-yl)-1-cyclopropyl-6-fluoro-5-hydroxy-8-methyl-1H-quinazolinedione;

7-(4-Aminomethyl-5,5-difluoro-4,5,6,7-tetrahydrobenzo[b]thiophen-2-yl)-1-cyclopropyl-6-fluoro-5-hydroxy-8-methyl-1H-quinazolinedione;

7-(4-Amino-5,5-difluorooctahydrocyclohepta[c]pyrrol-2-yl)-1-cyclopropyl-6-fluoro-5-hydroxy-8-methyl-1H-quinazolinedione;

1-Cyclopropyl-7-(5,5-difluoro-4-hydroxyoctahydrocyclohepta[c]pyrrol-2-yl)-6-fluoro-5-hydroxy-8-methyl-1H-quinazolinedione;

7-[5-(1-Aminoethyl)thiophen-3-yl]-1-cyclopropyl-6-fluoro-5-hydroxy-8-methyl-1H-quinazolinedione;

7-(5-Aminomethylthiophen-3-yl)-1-cyclopropyl-6-fluoro-5-hydroxy-8-methyl-1H-quinazolinedione;

7-(4-Amino-5,5-difluoro-4,5,6,7-tetrahydrobenzo[b]thiophen-2-yl)-1-cyclopropyl-6-fluoro-5-hydroxy-8-methyl-1H-quinazolinedione;

1-Cyclopropyl-7-(5,5-difluoro-4-hydroxy-4,5,6,7-tetrahydrobenzo[b]thiophen-2-yl)-6-fluoro-5-hydroxy-8-methyl-1H-quinazolinedione;

7-(4-Amino-5,5-difluorooctahydroisoindol-2-yl)-1-cyclopropyl-6-fluoro-5-methoxy-8-methyl-1H-quinazolinedione;

7-(4-Amino-5,5-difluorohexahydrocyclopenta[c]pyrrol-2-yl)-1-cyclopropyl-6-fluoro-5-methoxy-8-methyl-1H-quinazolinedione;

7-(5,5-Difluoro-4-hydroxyoctahydroisoindol-2-yl)-1-cyclopropyl-6-fluoro-5-methoxy-8-methyl-1H-quinazolinedione;

7-(5,5-Difluoro-4-hydroxyhexahydrocyclopenta[c]pyrrol-2-yl)-1-cyclopropyl-6-fluoro-5-methoxy-8-methyl-1H-quinazolinedione;

7-[3-(1-Amino-2-hydroxyethyl)pyrrolidin-1-yl]-1-cyclopropyl-6-fluoro-5-methoxy-8-methyl-1H-quinazolinedione;

7-[3-(1-Amino-3,3,3-trifluoro-2-trifluoromethylpropyl)pyrrolidin-1-yl]-1-cyclopropyl-6-fluoro-5-emthoxy-8-methyl-1H-quinazolinedione;

7-(4-Aminomethyl-4,5,6,7-tetrahydrobenzo[b]thiophen-2-yl)-1-cyclopropyl-6-fluoro-5-methoxy-8-methyl-1H-quinazolinedione;

1-Cyclopropyl-6-fluoro-7-(4-hydroxymethyl-4,5,6,7-tetrahydrobenzo[b]thiophen-2-yl)-5-methoxy-8-methyl-1H-quinazolinedione;

7-[4-(1-Aminoethyl)-4,5,6,7-tetrahydrobenzo[b]thiophen-2-yl]-1-cyclopropyl-6-fluoro-5-methoxy-8-methyl-1H-quinazolinedione;

7-[4-(2-Amino-1-hydroxyethyl)-4,5,6,7-tetrahydrobenzo[b]thiophen-2-yl]-1-cyclopropyl-6-fluoro-5-methoxy-8-methyl-1H-quinazolinedione;

7-[4-(1-Amino-2-hydroxyethyl)-4,5,6,7-tetrahydrobenzo[b]thiophen-2-yl]-1-cyclopropyl-6-fluoro-5-methoxy-8-methyl-1H-quinazolinedione;

7-[4-(1-Amino-2,2,2-trifluoroethyl)-4,5,6,7-tetrahydrobenzo[b]thiophen-2-yl]-1-cyclopropyl-6-fluoro-5-methoxy-8-methyl-1H-quinazolinedione;

7-(5-Aminomethyl-4,5,6,7-tetrahydrobenzo[b]thiophen-2-yl)-1-cyclopropyl-6-fluoro-5-methoxy-8-methyl-1H-quinazolinedione;

1-Cyclopropyl-6-fluoro-7-(5-hydroxymethyl-4,5,6,7-tetrahydrobenzo[b]thiophen-2-yl)-5-methoxy-8-methyl-1H-quinazolinedione;

7-[5-(1-Amino-2,2,2-trifluoroethyl)-4,5,6,7-tetrahydrobenzo[b]thiophen-2-yl]-1-cyclopropyl-6-fluoro-5-methoxy-8-methyl-1H-quinazolinedione;

7-[5-(1-Aminoethyl)-4,5,6,7-tetrahydrobenzo[b]thiophen-2-yl]-1-cyclopropyl-6-fluoro-5-methoxy-8-methyl-1H-quinazolinedione;

7-[5-(1-Amino-2-hydroxyethyl)-4,5,6,7-tetrahydrobenzo[b]thiophen-2-y]-1-cyclopropyl-6-fluoro-5-methoxy-8-methyl-1H-quinazolinedione;

7-(3-Aminomethyl-4-trifluoromethylpyrrolidin-1-yl)-1-cyclopropyl-6-fluoro-5-methoxy-8-methyl-1H-quinazolinedione;

7-[3-(1-Amino-2,2,3,3,3-pentafluoropropyl)pyrrolidin-1-yl]-1-cyclopropy-6-fluoro-5-methoxy-8-methyl-1H-quinazolinedione;

7-[3-(1-Amino-3,3,3-trifluoropropyl)pyrrolidin-1-yl-1-cyclopropyl-6-fluoro-5-methoxy-8-methyl-1H-quinazolinedione;

7-[3-(1-Amino-3,3,3-trifluoro-2-trifluoromethylpropyl)pyrrolidin-1-yl]-1-cyclopropyl-6-fluoro-5-methoxy-8-methyl-1H-quinazolinedione;

7-[4-(1-Aminoethyl)-3,3-difluoropyrrolidin-1-yl]-1-cyclopropyl-6-fluoro-5-methoxy-8-methyl-1H-quinazolinedione;

7-(3-Amino-4-ethylpiperidin-1-yl)-1-cyclopropyl-6-fluoro-5-methoxy-8-methyl-1H-quinazolinedione;

7-[3-(1-Amino-2-trifluoromethoxyethyl)pyrrolidin-1-yl]-1-cyclopropyl-6-fluoro-5-methoxy-8-methyl-1H-quinazolinedione;

7-(3-Aminomethyl-4-trifluoromethoxypyrrolidin-1-yl)-1-cyclopropyl-6-fluoro-5-methoxy-8-methyl-1H-quinazolinedione;

7-[3-Aminomethyl-4-(2,2,2-trifluoroethyl)pyrrolidin-1-yl]-1-cyclopropyl-6-fluoro-5-methoxy-8-methyl-1H-quinazolinedione;

7-[3-(1-Amino-2-difluoromethyl-3,3-difluoropropyl)pyrrolidin-1-yl]-1-cyclopropyl-6-fluoro-5-methoxy-8-methyl-1H-quinazolinedione;

7-[3(1-Amino-3-fluoro-2-fluoromethylpropyl)pyrrolidin-1-yl]-1-cyclopropyl-6-fluoro-5-methoxy-8-methyl-1H-quinazolinedione;

7-[3-(1-Amino-3,3-difluoropropyl)pyrrolidin-1-yl]-1-cyclopropyl-6-fluoro-5-methoxy-8-methyl-1H-quinazolinedione;

1-Cyclopropyl-7-(3,3-difluoro-4-hydroxymethylpyrrolidin-1-yl)-6-fluoro-5-methoxy-8-methyl-1H-quinazolinedione;

1-Cyclopropyl-7-[7-(1,2-dihydroxyethyl)-5-azaspiro[2.4]hept-5-yl]-6-fluoro-5-methoxy-8-methyl-1H-quinazolinedione;

1-Cyclopropyl-7-(5,5-difluoro-4-hydroxymethyl-4,5,6,7-tetrahydrobenzo[b]thiophen-2-yl)-6-fluoro-5-methoxy-8-methyl-1H-quinazolinedione;

7-[3-(1-Amino-2,2,2-trifluoro-1-trifluoromethylethyl)pyrrolidin-1-yl]-1-cyclopropyl-6-fluoro-5-methoxy-8-methyl-1H-quinazolinedione;

7-[3-Aminomethyl-4-(3,3,3-trifluoro-2-trifluoromethylpropyl)pyrrolidin-1-yl]-1-cyclopropyl-6-fluoro-5-methoxy-8-methyl-1H-quinazolinedione;

7-[3-(1-Amino-4,4,4-trifluoro-3-trifluoromethyl-but-2-enyl)pyrrolidin-1-yl]-1-cyclopropyl-6-fluoro-5-methoxy-8-methyl-1H-quinazolinedione;

7-[3-(1-Amino-4,4,4-trifluoro-3-trifluoromethylbutyl)pyrrolidin-1-yl]-1-cyclopropyl-6-fluoro-5-methoxy-8-methyl-1H-quinazolinedione;

7-[3-(1-Amino-2-hydroxyethyl)-4-fluoropyrrolidin-1-yl]-1-cyclopropyl-6-fluoro-5-methoxy-8-methyl-1H-quinazolinedione;

7-[3-(1-Amino-2,2,2-trifluoroethyl)-4-fluoropyrrolidin-1-yl]-1-cyclopropyl-6-fluoro-5-methoxy-8-methyl-1H-quinazolinedione;

7-(3-Aminomethyl-4-difluoromethoxypyrrolidin-1-yl)-1-cyclopropyl-6-fluoro-5-methoxy-8-methyl-1H-quinazolinedione;

7-(4-Aminomethyl-5,5-difluoro-4,5,6,7-tetrahydrobenzo[b]thiophen-2-yl)-1-cyclopropyl-6-fluoro-5-methoxy-8-methyl-1H-quinazolinedione;

7-(4-Amino-5,5-difluorooctahydrocyclohepta[c]pyrrol-2-yl)-1-cyclopropyl-6-fluoro-5-methoxy-8-methyl-1H-quinazolinedione;

1-Cyclopropyl-7-(5,5-difluoro-4-hydroxyoctahydrocyclohepta[c]pyrrol-2-yl)-6-fluoro-5-methoxy-8-methyl-1H-quinazolinedione;

7-[5-(1-Aminoethyl)thiophen-3-yl]-1-cyclopropyl-6-fluoro-5-hydroxy-8-methyl-1H-quinazolinedione;

7-(5-Aminomethylthiophen-3-yl)-1-cyclopropyl-6-fluoro-5-methoxy-8-methyl-1H-quinazolinedione;

7-(4-Amino-5,5-difluoro-4,5,6,7-tetrahydrobenzo[b]thiophen-2-yl)-1-cyclopropyl-6-fluoro-5-methoxy-8-methy]-1H-quinazolinedione;

1-Cyclopropyl-7-(5,5-difluoro-4-hydroxy-4,5,6,7-tetrahydrobenzo[b]thiophen-2-yl)-6-fluoro-5-methoxy-8-methyl-1H-quinazolinedione;

7-(4-Amino-5,5-difluorooctahydroisoindol-2-yl)-1-cyclopropyl-6-fluoro-8-methyl-1H-quinazolinedione;

7-(4-Amino-5,5-difluorohexahydrocyclopenta[c]pyrrol-2-yl)-1-cyclopropyl-6-fluoro-8-methyl-1H-quinazolinedione;

7-(5,5-Difluoro-4-hydroxyoctahydroisoindol-2-yl)-1-cyclopropyl-6-fluoro-8-methyl-1H-quinazolinedione;

7-(5,5-Difluoro-4-hydroxyhexahydrocyclopenta[c]pyrrol-2-yl)-1-cyclopropyl-6-fluoro-8-methyl-1H-quinazolinedione;

7-[3-(1-Amino-2-hydroxyethyl)pyrrolidin-1-yl]-1-cyclopropyl-6-fluoro-8-methyl-1H-quinazolinedione;

7-[3-(1-Amino-3,3,3-trifluoro-2-trifluoromethylpropyl)pyrrolidin-1-yl]-1-cyclopropyl-6-fluoro-8-methyl-1H-quinazolinedione;

7-(4-Aminomethyl-4,5,6,7-tetrahydrobenzo[b]thiophen-2-yl)-1-cyclopropyl-6-fluoro-8-methyl-1H-quinazolinedione;

1-Cyclopropyl-6-fluoro-7-(4-hydroxymethyl-4,5,6,7-tetrahydrobenzo[b]thiophen-2-yl)-8-methyl-1H-quinazolinedione;

7-[4-(1-Aminoethyl)-4,5,6,7-tetrahydrobenzo[b]thiophen-2-yl]-1-cyclopropyl-6-fluoro-8-methyl-1H-quinazolinedione;

7-[4-(2-Amino-1-hydroxyethyl)-4,5,6,7-tetrahydrobenzo[b]thiophen-2-yl]-1-cyclopropyl-6-fluoro-8-methyl-1H-quinazolinedione;

7-[4-(1-Amino-2-hydroxyethyl)-4,5,6,7-tetrahydrobenzo[b]thiophen-2-yl]-1-cyclopropyl-6-fluoro-8-methyl-1H-quinazolinedione;

7-[4-(1-Amino-2,2,2-trifluoroethyl)-4,5,6,7-tetrahydrobenzo[b]thiophen-2-yl]-1-cyclopropyl-6-fluoro-8-methyl-1H-quinazolinedione;

7-(5-Aminomethyl-4,5,6,7-tetrahydrobenzo[b]thiophen-2-yl)-1-cyclopropyl-6-fluoro-8-methyl-1H-quinazolinedione;

1-Cyclopropyl-6-fluoro-7-(5-hydroxymethyl-4,5,6,7-tetrahydrobenzo[b]thiophen-2-yl)-8-methyl-1H-quinazolinedione;

7-[5-(1-Amino-2,2,2-trifluoroethyl)-4,5,6,7-tetrahydrobenzo[b]thiophen-2-yl]-1-cyclopropyl-6-fluoro-8-methyl-1H-quinazolinedione;

7-[5-(1-Aminoethyl)-4,5,6,7-tetrahydrobenzo[b]thiophen-2-yl]-1-cyclopropyl-6-fluoro-8-methyl-1H-quinazolinedione;

7-[5-(1-Amino-2-hydroxyethyl)-4,5,6,7-tetrahydrobenzo[b]thiophen-2-yl]-1-cyclopropyl-6-fluoro-8-methyl-1H-quinazolinedione;

7-(3-Aminomethyl-4-trifluoromethylpyrrolidin-1-yl)-1-cyclopropyl-6-fluoro-8-methyl-1H-quinazolinedione;

7-[3-(1-Amino-2,2,3,3,3-pentafluoropropyl)pyrrolidin-1-yl]-1-cyclopropyl-6-fluoro-8-methyl-1H-quinazolinedione;

7-[3-(1-Amino-3,3,3-trifluoropropyl)pyrrolidin-1-yl]-1-cyclopropyl-6-fluoro-8-methyl-1H-quinazolinedione;

7-[3-(1-Amino-3,3,3-trifluoro-2-trifluoromethylpropyl)pyrrolidin-1-yl]-1-cyclopropyl-6-fluoro-8-methyl-1H-quinazolinedione;

7-[4-(1-Aminoethyl)-3,3-difluoropyrrolidin-1-yl]-1-cyclopropyl-6-fluoro-8-methyl-1H-quinazolinedione;

7-(3-Amino-4-ethylpiperidin-1-yl)-1-cyclopropyl-6-fluoro-8-methyl-1H-quinazolinedione;

7-[3-(1-Amino-2-trifluoromethoxyethyl)pyrrolidin-1-yl]-1-cyclopropyl-6-fluoro-8-methyl-1H-quinazolinedione;

7-(3-Aminomethyl-4-trifluoromethoxypyrrolidin-1-yl)-1-cyclopropyl-6-fluoro-8-methyl-1H-quinazolinedione;

7-[3-Aminomethyl-4-(2,2,2-trifluoroethyl)pyrrolidin-1-yl]-1-cyclopropyl-6-fluoro-8-methyl-1H-quinazolinedione;

7-[3-(1-Amino-2-difluoromethyl-3,3-difluoropropyl)pyrrolidin-1-yl]-1-cyclopropyl-6-fluoro-8-methyl-1H-quinazolinedione;

7-[3-(1-Amino-3-fluoro-2-fluoromethylpropyl)pyrrolidin-1-yl]-1-cyclopropyl-6-fluoro-8-methyl-1H-quinazolinedione

7-[3-(1-Amino-3,3-difluoropropyl)pyrrolidin-1-yl]-1-cyclopropyl-6-fluoro-8-methyl-1H-quinazolinedione;

1-Cyclopropyl-7-(3,3-difluoro-4-hydroxymethylpyrrolidin-1-yl)-6-fluoro-8-methyl-1H-quinazolinedione;

1-Cyclopropyl-7-[7-(1,2-dihydroxyethyl)-5-azaspiro[2.4]hept-5-yl]-6-fluoro-8-methyl-1H-quinazolinedione;

1-Cyclopropyl-7-(5,5-difluoro-4-hydroxymethyl-4,5,6,7-tetrahydrobenzo[b]thiophen-2-yl)-6-fluoro-8-methyl-1H-quinazolinedione;

7-[3-(1-Amino-2,2,2-trifluoro-1-trifluoromethylethyl)pyrrolidin-1-yl]-1-cyclopropyl-6-fluoro-8-methyl-1H-quinazolinedione;

7-[3-Aminomethyl-4-(3,3,3-trifluoro-2-trifluoromethylpropyl)pyrrolidin-1-yl]-1-cyclopropyl-6-fluoro-8-methyl-1H-quinazolinedione;

7-[3-(1-Amino-4,4,4-trifluoro-3-trifluoromethyl-but-2-enyl)pyrrolidin-1-yl]-1-cyclopropyl-6-fluoro-8-methyl-1H-quinazolinedione;

7-[3-(1-Amino-4,4,4-trifluoro-3-trifluoromethylbutyl)pyrrolidin-1-yl]-1-cyclopropyl-6-fluoro-8-methyl-1H-quinazolinedione;

7-[3-(1-Amino-2-hydroxyethyl)-4-fluoropyrrolidin-1-yl]-1-cyclopropyl-6-fluoro-8-methyl-1H-quinazolinedione;

7-[3-(1-Amino-2,2,2-trifluoroethyl)-4-fluoropyrrolidin-1-yl]-1-cyclopropyl-6-fluoro-8-methyl-1H-quinazolinedione;

7-(3-Aminomethyl-4-difluoromethoxypyrrolidin-1-yl)-1-cyclopropyl-6-fluoro-8-methyl-1H-quinazolinedione;

7-(4-Aminomethyl-5,5-difluoro-4,5,6,7-tetrahydrobenzo[b]thiophen-2-yl)-1-cyclopropyl-6-fluoro-8-methyl-1H-quinazolinedione;

7-(4-Amino-5,5-difluorooctahydrocyclohepta[c]pyrrol-2-yl)-1-cyclopropyl-6-fluoro-8-methyl-1H-quinazolinedione;

1-Cyclopropyl-7-(5,5-difluoro-4-hydroxyoctahydrocyclohepta[c]pyrrol-2-yl)-6-fluoro-8-methyl-1H-quinazolinedione;

7-[5-(1-Aminoethyl)thiophen-3-yl]-1-cyclopropyl-6-fluoro-8-methyl-1H-quinazolinedione;

7-(5-Aminomethylthiophen-3-yl)-1-cyclopropyl-6-fluoro-8-methyl-1H-quinazolinedione;

7-(4-Amino-5,5-difluoro-4,5,6,7-tetrahydrobenzo[b]thiophen-2-yl)-1-cyclopropyl-6-fluoro-8-methyl-1H-quinazolinedione

1-Cyclopropyl-7-(5,5-difluoro-4-hydroxy-4,5,6,7-tetrahydrobenzo[b]thiophen-2-yl)-6-fluoro-8-methyl-1H-quinazolinedione

7-(4-Amino-5,5-difluorooctahydroisoindol-2-yl)-1-cyclopropyl-6-fluoro-8-methoxy-1H-quinazolinedione;

7-(4-Amino-5,5-difluorohexahydrocyclopenta[c]pyrrol-2-yl)-1-cyclopropyl-6-fluoro-8-methoxy-1H-quinazolinedione;

7-(5,5-Difluoro-4-hydroxyoctahydroisoindol-2-yl)-1-cyclopropyl-6-fluoro-8-methoxy-1H-quinazolinedione;

7-(5,5-Difluoro-4-hydroxyhexahydrocyclopenta[c]pyrrol-2-yl)-1-cyclopropyl-6-fluoro-8-methoxy-1H-quinazolinedione;

7-[3-(1-Amino-2-hydroxyethyl)pyrrolidin-1-yl]-1-cyclopropyl-6-fluoro-8-methoxy-1H-quinazolinedione;

7-[3-(1-Amino-3,3,3-trifluoro-2-trifluoromethylpropyl)pyrrolidin-1-yl]-1-cyclopropyl-6-fluoro-8-methoxy-1H-quinazolinedione;

7-(4-Aminomethyl-4,5,6,7-tetrahydrobenzo[b]thiophen-2-yl)-1-cyclopropyl-6-fluoro-8-methoxy-1H-quinazolinedione;

1-Cyclopropyl-6-fluoro-7-(4-hydroxymethyl-4,5,6,7-tetrahydro-benzo[b]thiophen-2-yl)-8-methoxy-1H-quinazolinedione;

7-[4-(1-Aminoethyl)-4,5,6,7-tetrahydrobenzo[b]thiophen-2-yl]-1-cyclopropyl-6-fluoro-8-methoxy-1H-quinazolinedione;

7-[4-(2-Amino-1-hydroxyethyl)-4,5,6,7-tetrahydrobenzo[b]thiophen-2-yl]-1-cyclopropyl-6-fluoro-8-methoxy-1H-quinazolinedione;

7-[4-(1-Amino-2-hydroxyethyl)-4,5,6,7-tetrahydrobenzo[b]thiophen-2-yl]-1-cyclopropyl-6-fluoro-8-methoxy-1H-quinazolinedione;

7-[4-(1-Amino-2,2,2-trifluoroethyl)-4,5,6,7-tetrahydrobenzo[b]thiophen-2-yl]-1-cyclopropyl-6-fluoro-8-methoxy-1H-quinazolinedione;

7-(5-Aminomethyl-4,5,6,7-tetrahydrobenzo[b]thiophen-2-yl)-1-cyclopropyl-6-fluoro-8-methoxy-1H-quinazolinedione;

1-Cyclopropyl-6-fluoro-7-(5-hydroxymethyl-4,5,6,7-tetrahydrobenzo[b]thiophen-2-yl)-8-methoxy-1H-quinazolinedione;

7-[5-(1-Amino-2,2,2-trifluoroethyl)-4,5,6,7-tetrahydrobenzo[b]thiophen-2-yl]-1-cyclopropyl-6-fluoro-8-methoxy-1H-quinazolinedione;

7-[5-(1-Aminoethyl)-4,5,6,7-tetrahydrobenzo[b]thiophen-2-yl]-1-cyclopropyl-6-fluoro-8-methoxy-1H-quinazolinedione;

7-[5-(1-Amino-2-hydroxyethyl)-4,5,6,7-tetrahydrobenzo[b]thiophen-2-yl]-1-cyclopropyl-6-fluoro-8-methoxy-1H-quinazolinedione;

7-(3-Aminomethyl-4-trifluoromethylpyrrolidin-1-yl)-1-cyclopropyl-6-fluoro-8-methoxy-1H-quinazolinedione;

7-[3-(1-Amino-2,2,3,3,3-pentafluoropropyl)pyrrolidin-1-yl]-1-cyclopropyl-6-fluoro-8-methoxy-1H-quinazolinedione;

7-[3-(1-Amino-3,3,3-trifluoropropyl)pyrrolidin-1-yl]-1-cyclopropyl-6-fluoro-8-methoxy-1H-quinazolinedione;

7-[3-(1-Amino-3,3,3-trifluoro-2-trifluoromethylpropyl)pyrrolidin-1-yl]-1-cyclopropyl-6-fluoro-8-methoxy-1H-quinazolinedione;

7-[4-(1-Aminoethyl)-3,3-difluoropyrrolidin-1-yl]-1-cyclopropyl-6-fluoro-8-methoxy-1H-quinazolinedione;

7-(3-Amino-4-ethylpiperidin-1-yl)-1-cyclopropyl-6-fluoro-8-methoxy-1H-quinazolinedione;

7-[3-(1-Amino-2-trifluoromethoxyethyl)pyrrolidin-1-yl]-1-cyclopropyl-6-fluoro-8-methoxy-1H-quinazolinedione;

7-(3-Aminomethyl-4-trifluoromethoxypyrrolidin-1-yl)-1-cyclopropyl-6-fluoro-8-methoxy-1H-quinazolinedione;

7-[3-Aminomethyl-4-(2,2,2-trifluoroethyl)pyrrolidin-1-yl]-1-cyclopropyl-6-fluoro-8-methoxy-1H-quinazolinedione;

7-[3-(1-Amino-2-difluoromethyl-3,3-difluoropropyl)pyrrolidin-1-yl]-1-cyclopropyl-6-fluoro-8-methoxy-1H-quinazolinedione;

7-[3-(1-Amino-3-fluoro-2-fluoromethylpropyl)pyrrolidin-1-yl]-1-cyclopropyl-6-fluoro-8-methoxy-1H-quinazolinedione;

7-[3-(1-Amino-3,3-difluoropropyl)pyrrolidin-1-yl]-1-cyclopropyl-6-fluoro-8-methoxy-1H-quinazolinedione;

1-Cyclopropyl-7-(3,3-difluoro-4-hydroxymethylpyrrolidin-1-yl)-6-fluoro-8-methoxy-1H-quinazolinedione;

1-Cyclopropyl-7-[7-(1,2-dihydroxyethyl)-5-azaspiro[2.4]hept-5-yl]-6-fluoro-8-methoxy-1H-quinazolinedione;

1-Cyclopropyl-7-(5,5-difluoro-4-hydroxymethyl-4,5,6,7-tetrahydrobenzo[b]thiophen-2-yl)-6-fluoro-8-methoxy-1H-quinazolinedione;

7-[3-(1-Amino-2,2,2-trifluoro-1-trifluoromethylethyl)pyrrolidin-1-yl]-1-cyclopropyl-6-fluoro-8-methoxy-1H-quinazolinedione;

7-[3-Aminomethyl-4-(3,3,3-trifluoro-2-trifluoromethylpropyl)pyrrolidin-1-yl]-1-cyclopropyl-6-fluoro-8-methoxy-1H-quinazolinedione;

7-[3-(1-Amino-4,4,4-trifluoro-3-trifluoromethylbut-2-enyl)pyrrolidin 1-yl]-1-cyclopropyl-6-fluoro-8-methoxy-1H-quinazolinedione;

7-[3-(1-Amino-4,4,4-trifluoro-3-trifluoromethylbutyl)pyrrolidin-1-yl]-1-cyclopropyl-6-fluoro-8-methoxy-1H-quinazolinedione;

7-[3-(1-Amino-2-hydroxyethyl)-4-fluoropyrrolidin-1-yl]-1-cyclopropyl-6-fluoro-8-methoxy-1H-quinazolinedione;

7-[3-(1-Amino-2,2,2-trifluoroethyl)-4-fluoropyrrolidin-1-yl]-1-cyclopropyl-6-fluoro-8-methoxy-1H-quinazolinedione;

7-(3-Aminomethyl-4-difluoromethoxypyrrolidin-1-yl)-1-cyclopropyl-6-fluoro-8-methoxy-1H-quinazolinedione;

7-(4-Aminomethyl-5,5-difluoro-4,5,6,7-tetrahydrobenzo[b]thiophen-2-yl)-1-cyclopropyl-6-fluoro-8-methoxy-1H-quinazolinedione;

7-(4-Amino-5,5-difluorooctahydrocyclohepta[c]pyrrol-2-yl)-1-cyclopropyl-6-fluoro-8-methoxy-1H-quinazolinedione;

1-Cyclopropyl-7-(5,5-difluoro-4-hydroxyoctahydrocyclohepta[c]pyrrol-2-yl)-6-fluoro-8-methyl-1H-quinazolinedione;

7-[5-(1-Aminoethyl)thiophen-3-yl]-1-cyclopropyl-6-fluoro-8-methoxy-1H-quinazolinedione;

7-(5-Aminomethylthiophen-3-yl)-1-cyclopropyl-6-fluoro-8-methoxy-1H-quinazolinedione;

7-(4-Amino-5,5-difluoro-4,5,6,7-tetrahydrobenzo[b]thiophen-2-yl)-1-cyclopropyl-6-fluoro-8-methoxy-1H-quinazolinedione;

1-Cyclopropyl-7-(5,5-difluoro-4-hydroxy-4,5,6,7-tetrahydrobenzo[b]thiophen-2-yl)-6-fluoro-8-methoxy-1H-quinazolinedione;

1-Cyclopropyl-7-[3-(1,2-dihydroxy-2-methylpropyl)pyrrolidin-1-yl]-6-fluoro-5,8-dimethyl-1H-quinazolinedione;

1-Cyclopropyl-6-fluoro-5,8-dimethyl-7-[3-(3,3,3-trifluoro-1,2-dihydroxy-2-trifluoromethylpropyl)pyrrolidin-1-yl]-1H-quinazolinedione;

1-Cyclopropyl-6-fluoro-7-{3-[hydroxy(1-hydroxycyclopropyl)methyl]pyrrolidin-1-yl}-5,8-dimethyl-1H-quinazolinedione;

1-Cyclopropyl-6-fluoro-7-{3-[hydroxy(1-hydroxycyclopentyl)methyl]pyrrolidin-1-yl}-5,8-dimethyl-1H-quinazolinedione;

7-[3-(1-Amino-2-hydroxy-2-methylpropyl)pyrrolidin-1-yl]-1-cyclopropyl, 6-fluoro-5,8-dimethyl-1H-quinazolinedione;

7-[3-(1-Amino-3,3,3-trifluoro-2-hydroxy-2-trifluoromethylpropyl)pyrrolidin-1-yl]-1-cyclopropyl-6-fluoro-5,8-dimethyl-1H-quinazolinedione;

7-{3-[Amino-(1-hydroxycyclopropyl)methyl]pyrrolidin-1-yl}-1-cyclopropyl-6-fluoro-5,8-dimethyl-1H-quinazolinedione;

7-{3-[Amino-(1-hydroxycyclopentyl)methyl]pyrrolidin-1-yl}-1-cyclopropyl-6-fluoro-5,8-dimethyl-1H-quinazolinedione;

1-Cyclopropyl-7-(4,5-dihydroxyhexahydrocyclopenta[c]pyrrol-2-yl)-6-fluoro-5,8-dimethyl-1H-quinazolinedione;

1-Cyclopropyl-7-(4,5-dihydroxyoctahydroisoindol-2-yl)-6-fluoro-5,8-dimethyl-1H-quinazolinedione;

1-Cyclopropyl-7-(4,5-dihydroxyoctahydrocyclohepta[c]pyrrol-2-yl)-6-fluoro-5,8-dimethyl-1H-quinazolinedione;

1-Cyclopropyl-7-(4,5-dihydroxydecahydrocycloocta[c]pyrrol-2-yl)-6-fluoro-5,8-dimethyl-1H-quinazolinedione;

5-Amino-1-cyclopropyl-7-[3-(1,2-dihydroxy-2-methylpropyl)pyrrolidin-1-yl]-6-fluoro-8-methyl-1H-quinazolinedione;

5-Amino-1-cyclopropyl-6-fluoro-8-methyl-7-[3-(3,3,3-trifluoro-1,2-dihydroxy-2-trifluoromethylpropyl)pyrrolidin-1-yl]-1H-quinazolinedione;

5-Amino-1-cyclopropyl-6-fluoro-7-{3-[hydroxy-(1-hydroxycyclopropyl)methyl]pyrrolidin-1-yl}-8-methyl-1H-quinazolinedione;

5-Amino-1-cyclopropyl-6-fluoro-7-{3-[hydroxy-(1-hydroxycyclopentyl)methyl]pyrrolidin-1-yl}-8-methyl-1H-quinazolinedione;

5-Amino-7-[3-(1-amino-2-hydroxy-2-methylpropyl)pyrrolidin-1-yl]-1-cyclopropyl-6-fluoro-8-methyl-1H-quinazolinedione;

5-Amino-7-[3-(1-amino-3,3,3-trifluoro-2-hydroxy-2-trifluoromethylpropyl)pyrrolidin-1-yl]-1-cyclopropyl-6-fluoro-8-methyl-1H-quinazolinedione;

5-Amino-7-{3-[amino-(1-hydroxycyclopropyl)methyl]pyrrolidin-1-yl}-1-cyclopropyl-6-fluoro-8-methyl-1H-quinazolinedione;

5-Amino-7-{3-[amino-(1-hydroxycyclopentyl)methyl]pyrrolidin-1-yl}-1-cyclopropyl-6-fluoro-8-methyl-1H-quinazolinedione;

5-Amino-1-cyclopropyl-7-(4,5-dihydroxyhexahydrocyclopenta[c]pyrrol-2-yl)-6-fluoro-8-methyl-1H-quinazolinedione;

5-Amino-1-cyclopropyl-7-(4,5-dihydroxyoctahydroisoindol-2-yl)-6-fluoro-8-methyl-1H-quinazolinedione;

5-Amino-1-cyclopropyl-7-(4,5-dihydroxyoctahydrocyclohepta[c]pyrrol-2-yl)-6-fluoro-8-methyl-1H-quinazolinedione;

5-Amino-1-cyclopropyl-7-(4,5-dihydroxydecahydrocycloocta[c]pyrrol-2-yl)-6-fluoro-8-methyl-1H-quinazolinedione;

1-Cyclopropyl-5-difluoromethyl-7-[3-(1,2-dihydroxy-2-methylpropyl)pyrrolidin-1-yl]-6-fluoro-8-methyl-1H-quinazolinedione;

1-Cyclopropyl-5-difluoromethyl-6-fluoro-8-methyl-7-[3-(3,3,3-trifluoro-1,2-dihydroxy-2-trifluoromethylpropyl)pyrrolidin-1-yl]-1H-quinazolinedione;

1-Cyclopropyl-5-difluoromethyl-6-fluoro-7-{3-[hydroxy-(1-hydroxycyclopropyl)methyl]pyrrolidin-1-yl}-8-methyl-1H-quinazolinedione;

1-Cyclopropyl-5-difluoromethyl-6-fluoro-7-{3-[hydroxy-(1-hydroxycyclopentyl)methyl]pyrrolidin-1-yl}-8-methyl-1H-quinazolinedione;

7-[3-(1-Amino-2-hydroxy-2-methylpropyl)pyrrolidin-1-yl]-1-cyclopropyl-5-difluoromethyl-6-fluoro-8-methyl-1H-quinazolinedione;

7-[3-(1-Amino-3,3,3-trifluoro-2-hydroxy-2-trifluoromethylpropyl)pyrrolidin-1-yl]-1-cyclopropyl-5-difluoromethyl-6-fluoro-8-methyl-1H-quinazolinedione;

7-{3-[Amino-(1-hydroxycyclopropyl)methyl]pyrrolidin-1-yl}-1-cyclopropyl-5-difluoromethyl-6-fluoro-8-methyl-1H-quinazolinedione;

7-{3-[Amino-(1-hydroxycyclopentyl)methyl]pyrrolidin-1-yl}-1-cyclopropyl-5-difluoromethyl-6-fluoro-8-methyl-1H-quinazolinedione;

1-Cyclopropyl-5-difluoromethyl-7-(4,5-dihydroxyhexahydrocyclopenta[c]pyrrol-2-yl)-6-fluoro-8-methyl-1H-quinazolinedione;

1-Cyclopropyl-5-difluoromethyl-7-(4,5-dihydroxyoctahydroisoindol-2-yl)-6-fluoro-8-methyl-1H-quinazolinedione;

1-Cyclopropyl-5-difluoromethyl-7-(4,5-dihydroxyoctahydrocyclohepta[c]pyrrol-2-yl)-6-fluoro-8-methyl-1H-quinazolinedione;

1-Cyclopropyl-5-difluoromethyl-7-(4,5-dihydroxydecahydrocycloocta[c]pyrrol-2-yl)-6-fluoro-8-methyl-1H-quinazolinedione;

1-Cyclopropyl-6-fluoro-7-(3-fluoro-4-hydroxymethylpyrrolidin-1-yl)-5,8-dimethyl-1H-quinazolinedione;

1-Cyclopropyl-7-(3,4-dihydroxypiperidin-1-yl)-6-fluoro-5,8-dimethyl-1H-quinazolinedione;

1-Cyclopropyl-6-fluoro-7-(3-hydroxymethyl-4-methylpiperidin-1-yl)-5,8-dimethyl-1H-quinazolinedione;

1-Cyclopropyl-7-[3-(,2-dihydroxy-2-methylpropyl)-4-fluoropyrrolidin-1-yl]-6-fluoro-5,8-dimethyl-1H-quinazolinedione;

1-Cyclopropyl-6-fluoro-7-{3-fluoro-4-[hydroxy-(1-hydroxycyclopropyl)methyl]pyrrolidin-1-yl}-5,8-dimethyl-1H-quinazolinedione;

1-Cyclopropyl-6-fluoro-7-{3-fluoro-4-[hydroxy-(1-hydroxycyclopentyl)methyl]pyrrolidin-1-yl}-5,8-dimethyl-1H-quinazolinedione;

1-Cyclopropyl-6-fluoro-7-[3-fluoro-4-(3,3,3-trifluoro-1,2-dihydroxy-2-trifluoromethylpropyl)pyrrolidin-1-yl]-5,8-dimethyl-1H-quinazolinedione;

1-Cyclopropyl-7-(4-ethyl-3-hydroxypiperidin-1-yl)-6-fluoro-5,8-dimethyl-1H-quinazolinedione;

1-Cyclopropyl-7-(4-ethyl-3-hydroxymethylpiperidin-1-yl)-6-fluoro-5,8-dimethyl-1H-quinazolinedione;

1-Cyclopropyl-6-fluoro-7-(3-hydroxy-4-methylpiperidin-1-yl)-5,8-dimethyl-1H-quinazolinedione;

1-Cyclopropyl-6-fluoro-5,8-dimethyl-7-[3-(2,2,2-trifluoro-1-hydroxyethyl)pyrrolidin-1-yl]-1H-quinazolinedione;

1-Cyclopropyl-7-[3-(1,2-dihydroxyethyl)-4-fluoropyrrolidin-1-yl]-6-fluoro-5,8-dimethyl-1H-quinazolinedione;

1-Cyclopropyl-6-fluoro-7-(4-hydroxy-3-hydroxymethylpiperidin-1-yl)-5,8-dimethyl-1H-quinazolinedione;

5-Amino-1-cyclopropyl-6-fluoro-7-(3-fluoro-4-hydroxymethylpyrrolidin-1-yl)-8-methyl-1H-quinazolinedione;

5-Amino-1-cyclopropyl-7-[3-(1,2-dihydroxyethyl)-4-fluoropyrrolidin-1-yl]-6-fluoro-8-methyl-1H-quinazolinedione;

5-Amino-1-cyclopropyl-6-fluoro-8-methyl-7-[3-(2,2,2-trifluoro-1-hydroxyethyl)pyrrolidin-1-yl]-1H-quinazolinedione;

5-Amino-1-cyclopropyl-7-(3,4-dihydroxypiperidin-1-yl)-6-fluoro-8-methyl-1H-quinazolinedione;

1-Cyclopropyl-6-fluoro-7-(4-hydroxy-3-hydroxymethylpiperidin-1-yl)-5,8-dimethyl-1H-quinazolinedione;

5-Amino-1-cyclopropyl-6-fluoro-7-(3-hydroxymethyl-4-methylpiperidin-1-yl)-8-methyl-1H-quinazolinedione;

5-Amino-1-cyclopropyl-7-[3-(,2-dihydroxy-2-methylpropyl)-4-fluoropyrrolidin-1-yl]-6-fluoro-8-methyl-1H-quinazolinedione;

5-Amino-1-cyclopropyl-6-fluoro-7-{3-fluoro-4-[hydroxy-(1-hydroxycyclopropyl)methyl]pyrrolidin-1-yl}-8-methyl-1H-quinazolinedione;

5-Amino-1-cyclopropyl-6-fluoro-7-(3-hydroxy-4-methylpiperidin-1-yl)-8-methyl-1H-quinazolinedione;

5-Amino-1-cyclopropyl-7-(4-ethyl-3-hydroxypiperidin-1-yl)-6-fluoro-8-methyl-1H-quinazolinedione;

5-Amino-1-cyclopropyl-7-(4-ethyl-3-hydroxymethylpiperidin-1-yl)-6-fluoro-8-methyl-1H-quinazolinedione;

5-Amino-1-cyclopropyl-6-fluoro-7-[3-fluoro-4-(3,3,3-trifluoro-1,2-dihydroxy-2-trifluoromethylpropyl)pyrrolidin-1-yl]-8-methyl-1H-quinazolinedione;

5-Amino-1-cyclopropyl-6-fluoro-7-{3-fluoro-4-[hydroxy-(1-hydroxycyclopentyl)methyl]pyrrolidin-1-yl}-8-methyl-1H-quinazolinedione;

1-Cyclopropyl-5-difluoromethyl-6-fluoro-7-{3-fluoro-4-[hydroxy-(1-hydroxycyclopropyl)methyl]pyrrolidin-1-yl}-8-methyl-1H-quinazolinedione;

1-Cyclopropyl-5-difluoromethyl-7-[3-(1,2-dihydroxy-2-methylpropyl)-4-fluoropyrrolidin-1-yl]-6-fluoro-8-methyl-1H-quinazolinedione;

1-Cyclopropyl-5-difluoromethyl-6-fluoro-7-(3-hydroxymethyl-4-methylpiperidin-1-yl)-8-methyl-1H-quinazolinedione;

1-Cyclopropyl-5-difluoromethyl-7-(3,4-dihydroxypiperidin-1-yl)-6-fluoro-8-methyl-1H-quinazolinedione;

1-Cyclopropyl-5-difluoromethyl-6-fluoro-7-(3-fluoro-4-hydroxymethylpyrrolidin-1-yl)-8-methyl-1H-quinazolinedione;

1-Cyclopropyl-5-difluoromethyl-6-fluoro-7-{3-fluoro-4-[hydroxy-(1-hydroxycyclopentyl)methyl]pyrrolidin-1-y}-8-methyl-1H-quinazolinedione;

1-Cyclopropyl-5-difluoromethyl-6-fluoro-7-[3-fluoro-4-(3,3,3-trifluoro-1,2-dihydroxy-2-trifluoromethylpropyl) pyrrolidin-1-yl]-8-methyl-1H-quinazolinedione;

1-Cyclopropyl-5-difluoromethyl-7-(4-ethyl-3-hydroxymethylpiperidin-1-yl)-6-fluoro-8-methyl-1H-quinazolinedione;

1-Cyclopropyl-5-difluoromethyl-7-(4-ethyl-3-hydroxypiperidin-1-yl)-6-fluoro-8-methyl-1H-quinazolinedione;

1-Cyclopropyl-5-difluoromethyl-6-fluoro-7-(4-hydroxy-3-hydroxymethylpiperidin-1-yl)-8-methyl-1H-quinazolinedione;

1-Cyclopropyl-5-difluoromethyl-6-fluoro-7-(3-hydroxy-4-methylpiperidin-1-yl)-8-methyl-1H-quinazolinedione;

1-Cyclopropyl-5-difluoromethyl-7-[3-(1,2-dihydroxyethyl)-4-fluoropyrrolidin-1-yl]-6-fluoro-8-methyl-1H-quinazolinedione;

1-Cyclopropyl-5-difluoromethyl-6-fluoro-8-methyl-7-[3-(2,2,2-trifluoro-1-hydroxyethyl)pyrrolidin-1-yl]-1H-quinazolinedione;

1-Cyclopropyl-7-[3-(,2-dihydroxy-2-methylpropyl)-pyrrolidin-1-yl]-6-fluoro-8-methoxy-5-methyl-1H-quinazolinedione;

1-Cyclopropyl-6-fluoro-8-methoxy-5-methyl-7-[3-(3,3,3-trifluoro-1,2-dihydroxy-2-trifluoromethylpropyl)pyrrolidin-1-yl]-1H-quinazolinedione;

1-Cyclopropyl-6-fluoro-7-{3-[hydroxy-(1-hydroxycyclopropyl)methyl]-pyrrolidin-1-yl}-8-methoxy-5-methyl-1H-quinazolinedione;

1-Cyclopropyl-6-fluoro-7-{3-[hydroxy-(1-hydroxycyclopentyl)-methyl]pyrrolidin-1-yl}-8-methoxy-5-methyl-1H-quinazolinedione;

7-[3-(1-Amino-2-hydroxy-2-methylpropyl)-pyrrolidin-1-yl]-1-cyclopropyl-6-fluoro-8-methoxy-5-methyl-1H-quinazolinedione;

7-[3-(1-Amino-3,3,3-trifluoro-2-hydroxy-2-trifluoromethylpropyl)pyrrolidin-1-yl]-1-cyclopropyl-6-fluoro-8-methoxy-5-methyl-1H-quinazolinedione;

7-{3-[Amino-(1-hydroxycyclopropyl)methyl]pyrrolidin-1-yl}-1-cyclopropyl-6-fluoro-8-methoxy-5-methyl-1H-quinazolinedione;

7-{3-[Amino-(1-hydroxycyclopentyl)methyl]pyrrolidin-1-yl}-1-cyclopropyl-6-fluoro-8-methoxy-5-methyl-1H-quinazolinedione;

1-Cyclopropyl-7-(4,5-dihydroxyhexahydrocyclopenta[c]pyrrol-2-yl)-6-fluoro-8-methoxy-5-methyl-1H-quinazolinedione;

1-Cyclopropyl-7-(4,5-dihydroxyoctahydroisoindol-2-yl)-6-fluoro-8-methoxy-5-methyl-1H-quinazolinedione;

1-Cyclopropyl-7-(4,5-dihydroxyoctahydrocyclohepta[c]pyrrol-2-yl)-6-fluoro-8-methoxy-5-methyl-1H-quinazolinedione;

1-Cyclopropyl-7-(4,5-dihydroxydecahydrocycloocta[c]pyrrol-2-yl)-6-fluoro-8-methoxy-5-methyl-1H-quinazolinedione;

5-Amino-1-cyclopropyl-7-[3-(1,2-dihydroxy-2-methylpropyl)pyrrolidin-1-yl]-6-fluoro-8-methoxy-1H-quinazolinedione;

5-Amino-1-cyclopropyl-6-fluoro-8-methoxy-7-[3-(3,3,3-trifluoro-1,2-dihydroxy-2-trifluoromethylpropyl)pyrrolidin-1-yl]-1H-quinazolinedione;

5-Amino-1-cyclopropyl-6-fluoro-7-{3-[hydroxy-(1-hydroxycyclopropyl)methyl]-pyrrolidin-1-yl}-8-methoxy-1H-quinazolinedione;

5-Amino-1-cyclopropyl-6-fluoro-7-{3-[hydroxy-(1-hydroxycyclopentyl)methyl]-pyrrolidin-1-yl}-8-methoxy-1H-quinazolinedione;

5-Amino-7-[3-(1-amino-2-hydroxy-2-methylpropyl)pyrrolidin-1-yl]-1-cyclopropyl-6-fluoro-8-methoxy-1H-quinazolinedione;

5-Amino-7-[3-(1-amino-3,3,3-trifluoro-2-hydroxy-2-trifluoromethylpropyl)pyrrolidin-1-yl]-1-cyclopropyl-6-fluoro-8-methoxy-1H-quinazolinedione;

5-Amino-7-{3-[amino-(1-hydroxycyclopropyl)methyl]pyrrolidin-1-y-1-cyclopropyl-6-fluoro-8-methoxy-1H-quinazolinedione;

5-Amino-7-{3-[amino-(1-hydroxycyclopentyl)methyl]pyrrolidin-1-yl}-1-cyclopropyl-6-fluoro-8-methoxy-1H-quinazolinedione;

5-Amino-1-cyclopropyl-7-(4,5-dihydroxyhexahydrocyclopenta[c]pyrrol-2-yl)-6-fluoro-8-methoxy-1H-quinazolinedione;

5-Amino-1-cyclopropyl-7-(4,5-dihydroxyoctahydroisoindol-2-yl)-6-fluoro-8-methoxy-1H-quinazolinedione;

5-Amino-1-cyclopropyl-7-(4,5-dihydroxyoctahydrocyclohepta[c]pyrrol-2-yl)-6-fluoro-8-methoxy-1H-quinazolinedione;

5-Amino-1-cyclopropyl-7-(4,5-dihydroxydecahydrocycloocta[c]pyrrol-2-yl)-6-fluoro-8-methoxy-1H-quinazolinedione;

1-Cyclopropyl-5-difluoromethyl-7-[3-(1,2-dihydroxy-2-methylpropyl)pyrrolidin-1-yl]-6-fluoro-8-methoxy-1H-quinazolinedione;

1-Cyclopropyl-5-difluoromethyl-6-fluoro-8-methoxy-7-[3-(3,3,3-trifluoro-1,2-dihydroxy-2-trifluoromethylpropyl)pyrrolidin-1-yl]-1H-quinazolinedione;

1-Cyclopropyl-5-difluoromethyl-6-fluoro-7-{3-[hydroxy-(1-hydroxy-cyclopropyl)methyl]pyrrolidin-1-yl}-8-methoxy-1H-quinazolinedione;

1-Cyclopropyl-5-difluoromethyl-6-fluoro-7-{3-[hydroxy-(1-hydroxycyclopentyl)methyl]pyrrolidin-1-yl}-8-methoxy-1H-quinazolinedione;

7-[3-(1-Amino-2-hydroxy-2-methylpropyl)pyrrolidin-1-yl]-1-cyclopropyl-5-difluoromethyl-6-fluoro-8-methoxy-1H-quinazolinedione;

7-[3-(1-Amino-3,3,3-trifluoro-2-hydroxy-2-trifluoromethylpropyl)pyrrolidin-1-yl]-1-cyclopropyl-5-difluoromethyl-6-fluoro-8-methoxy-1H-quinazolinedione;

7-{3-[Amino-(1-hydroxycyclopropyl)methyl]pyrrolidin-1-yl}-1-cyclopropyl-5-difluoromethyl-6-fluoro-8-methoxy-1H-quinazolinedione;

7-{3-[Amino-(1-hydroxycyclopentyl)methyl]pyrrolidin-1-yl}-1-cyclopropyl-5-difluoromethyl-6-fluoro-8-methoxy-1H-quinazolinedione;

1-Cyclopropyl-5-difluoromethyl-7-(4,5-dihydroxyhexahydrocyclopenta[c]pyrrol-2-yl)-6-fluoro-8-methoxy-1H-quinazolinedione;

1-Cyclopropyl-5-difluoromethyl-7-(4,5-dihydroxyoctahydroisoindol-2-yl)-6-fluoro-8-methoxy-1H-quinazolinedione;

1-Cyclopropyl-5-difluoromethyl-7-(4,5-dihydroxyoctahydrocyclohepta[c]pyrrol-2-yl)-6-fluoro-8-methoxy-1H-quinazolinedione;

1-Cyclopropyl-5-difluoromethyl-7-(4,5-dihydroxydecahydrocycloocta[c]pyrrol-2-yl)-6-fluoro-8-methoxy-1H-quinazolinedione;

1-Cyclopropyl-6-fluoro-7-(3-fluoro-4-hydroxymethylpyrrolidin-1-yl)-8-methoxy-5-methyl-1H-quinazolinedione;

1-Cyclopropyl-7-[3-(1,2-dihydroxyethyl)-4-fluoropyrrolidin-1-yl]-6-fluoro-8-methoxy-5-methyl-1H-quinazolinedione;

1-Cyclopropyl-6-fluoro-8-methoxy-5-methyl-7-[3-(2,2,2-trifluoro-1-hydroxyethyl)pyrrolidin-1-yl]-1H-quinazolinedione;

1-Cyclopropyl-7-(3,4-dihydroxypiperidin-1-yl)-6-fluoro-8-methoxy-5-methyl-1H-quinazolinedione;

1-Cyclopropyl-6-fluoro-7-(4-hydroxy-3-hydroxymethylpiperidin-1-yl)-8-methoxy-5-methyl-1H-quinazolinedione;

1-Cyclopropyl-6-fluoro-7-(3-hydroxy-4-methylpiperidin-1-yl)-8-methoxy-5-methyl-1H-quinazolinedione;

1-Cyclopropyl-6-fluoro-7-(3-hydroxymethyl-4-methylpiperidin-1-yl)-8-methoxy-5-methyl-1H-quinazolinedione;

1-Cyclopropyl-7-(4-ethyl-3-hydroxymethylpiperidin-1-yl)-6-fluoro-8-methoxy-5-methyl-1H-quinazolinedione;

1-Cyclopropyl-7-(4-ethyl-3-hydroxypiperidin-1-yl)-6-fluoro-8-methoxy-5-methyl-1H-quinazolinedione;

1-Cyclopropyl-7-[3-(1,2-dihydroxy-2-methylpropyl)-4-fluoropyrrolidin-1-yl]-6-fluoro-8-methoxy-5-methyl-1H-quinazolinedione;

1-Cyclopropyl-6-fluoro-7-[3-fluoro-4-(3,3,3-trifluoro-1,2-dihydroxy-2-trifluoromethylpropyl)-pyrrolidin-1-yl]-8-methoxy-5-methyl-1H-quinazolinedione;

1-Cyclopropyl-6-fluoro-7-{3-fluoro-4-[hydroxy-(1-hydroxycyclopropyl)methyl]-pyrrolidin-1-yl}-8-methoxy-5-methyl-1H-quinazolinedione;

1-Cyclopropyl-6-fluoro-7-{3-fluoro-4-[hydroxy-(1-hydroxycyclopentyl)methyl]pyrrolidin-1-yl}-8-methoxy-5-methyl-1H-quinazolinedione;

5-Amino-1-cyclopropyl-6-fluoro-7-(3-fluoro-4-hydroxymethylpyrrolidin-1-yl)-8-methoxy-1H-quinazolinedione;

5-Amino-1-cyclopropyl-7-[3-(1,2-dihydroxyethyl)-4-fluoropyrrolidin-1-yl]-6-fluoro-8-methoxy-1H-quinazolinedione;

5-Amino-1-cyclopropyl-6-fluoro-8-methoxy-7-[3-(2,2,2-trifluoro-1-hydroxyethyl)pyrrolidin-1-yl]-1H-quinazolinedione;

5-Amino-1-cyclopropyl-7-(3,4-dihydroxypiperidin-1-yl)-6-fluoro-8-methoxy-1H-quinazolinedione;

5-Amino-1-cyclopropyl-6-fluoro-7-(4-hydroxy-3-hydroxymethylpiperidin-1-yl)-8-methoxy-1H-quinazolinedione;

5-Amino-1-cyclopropyl-6-fluoro-7-(3-hydroxy-4-methylpiperidin-1-yl)-8-methoxy-1H-quinazolinedione;

5-Amino-1-cyclopropyl-6-fluoro-7-(3-hydroxymethyl-4-methylpiperidin-1-yl)-8-methoxy-1H-quinazolinedione;

5-Amino-1-cyclopropyl-7-(4-ethyl-3-hydroxymethylpiperidin-1-yl)-6-fluoro-8-methoxy-1H-quinazolinedione;

5-Amino-1-cyclopropyl-7-(4-ethyl-3-hydroxypiperidin-1-yl)-6-fluoro-8-methoxy-1H-quinazolinedione;

5-Amino-1-cyclopropyl-7-[3-(1,2-dihydroxy-2-methylpropyl)-4-fluoropyrrolidin-1-yl]-6-fluoro-8-methoxy-1H-quinazolinedione;

5-Amino-1-cyclopropyl-6-fluoro-7-[3-fluoro-4-(3,3,3-trifluoro-1,2-dihydroxy-2-trifluoromethylpropyl)pyrrolidin-1-yl]-8-methoxy-1H-quinazolinedione;

5-Amino-1-cyclopropyl-6-fluoro-7-{3-fluoro-4-[hydroxy-(1-hydroxycyclopropyl)methyl]pyrrolidin-1-yl}-8-methoxy-1H-quinazolinedione;

5-Amino-1-cyclopropyl-6-fluoro-7-{3-fluoro-4-[hydroxy-(1-hydroxycyclopentyl)methyl]pyrrolidin-1-yl}-8-methoxy-1H-quinazolinedione;

1-Cyclopropyl-5-difluoromethyl-6-fluoro-7-(3-fluoro-4-hydroxymethylpyrrolidin-1-yl)-8-methoxy-1H-quinazolinedione;

1-Cyclopropyl-5-difluoromethyl-7-[3-(1,2-dihydroxyethyl)-4-fluoropyrrolidin-1-yl]-6-fluoro-8-methoxy-1H-quinazolinedione;

1-Cyclopropyl-5-difluoromethyl-6-fluoro-8-methoxy-7-[3-(2,2,2-trifluoro-1-hydroxyethyl)pyrrolidin-1-yl]-1H-quinazolinedione;

1-Cyclopropyl-5-difluoromethyl-7-(3,4-dihydroxypiperidin-1-yl)-6-fluoro-8-methoxy-1H-quinazolinedione;

1-Cyclopropyl-5-difluoromethyl-6-fluoro-7-(4-hydroxy-3-hydroxymethylpiperidin-1-yl)-8-methoxy-1H-quinazolinedione;

1-Cyclopropyl-5-difluoromethyl-6-fluoro-7-(3-hydroxy-4-methylpiperidin-1-yl)-8-methoxy-1H-quinazolinedione;

1-Cyclopropyl-5-difluoromethyl-6-fluoro-7-(3-hydroxymethyl-4-methylpiperidin-1-yl)-8-methoxy-1H-quinazolinedione;

1-Cyclopropyl-5-difluoromethyl-7-(4-ethyl-3-hydroxymethylpiperidin-1-yl)-6-fluoro-8-methoxy-1H-quinazolinedione;

1-Cyclopropyl-5-difluoromethyl-7-(4-ethyl-3-hydroxypiperidin-1-yl)-6-fluoro-8-methoxy-1H-quinazolinedione;

1-Cyclopropyl-5-difluoromethyl-7-[3-(1,2-dihydroxy-2-methylpropyl)-4-fluoropyrrolidin-1-yl]-6-fluoro-8-methoxy-1H-quinazolinedione;

1-Cyclopropyl-5-difluoromethyl-6-fluoro-7-[3-fluoro-4-(3,3,3-trifluoro-1,2-dihydroxy-2-trifluoromethylpropyl)pyrrolidin-1-yl]-8-methoxy-1H-quinazolinedione;

1-Cyclopropyl-5-difluoromethyl-6-fluoro-7-{3-fluoro-4-[hydroxy-(1-hydroxycyclopropyl)methyl]pyrrolidin-1-yl}-8-methoxy-1H-quinazolinedione;

1-Cyclopropyl-5-difluoromethyl-6-fluoro-7-{3-fluoro-4-[hydroxy-(1-hydroxycyclopentyl)methyl]pyrrolidin-1-yl}-8-methoxy-1H-quinazolinedione; or a pharmaceutically acceptable salt thereof.

The invention also provides a pharmaceutical composition comprising a compound of one of the above-mentioned Formulas admixed with a carrier, diluent, or excipient.

The invention also provides a method of treating a bacterial infection in a mammal comprising administering to the mammal in need thereof an antibacterial effective amount of a compound of one of the above-mentioned Formulas.

The invention also provides a method of inhibiting a bacterial topoisomerase in a mammal comprising administering to the mammal in need thereof an effective amount of a compound of one of the above-mentioned Formulas.

The invention also provides a method of inhibiting a bacterial DNA gyrase in a mammal comprising administering to the mammal in need thereof an effective amount of a compound of one of the above-mentioned Formulas.

The invention also provides a method of inhibiting a bacterial topoisomerase IV in a mammal comprising administering to the mammal in need thereof an effective amount of a compound of one of the above-mentioned Formulas.

The invention also provides a method of inhibiting a quinolone-resistant bacteria in a mammal comprising administering to the mammal an effective amount of a compound of one of the above-mentioned Formulas.

The invention also provides a method of inhibiting a quinolone resistant bacterial DNA gyrase in a mammal comprising administering to the mammal an effective amount of a compound of any of Formulas I-VIII.

A method of inhibiting a quinolone resistant bacterial topoisomerase in a mammal comprising administering to the mammal an effective amount of a compound of any of Formulas I-VIII.

The invention also provides a process for preparing a compound of formula IX, wherein R₁, R₂, R₃, R₄, R₆, J, K, V, V′, z, and R′ are as defined above and R_{5′} is halo, comprising:

(a) coupling compound IXA wherein M is n-Bu₃Sn with compound IXB wherein R_{5′} is halo in the presence of Pd° to provide the R5-coupled product IXC;

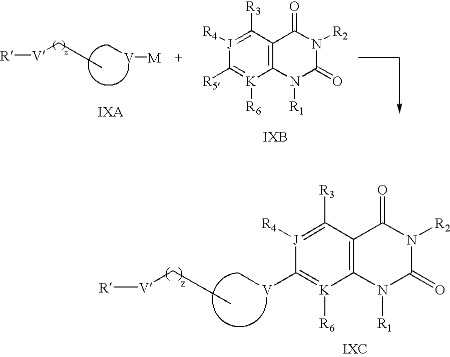

(b) removing the R′ group in IXC to provide compound IXD; and

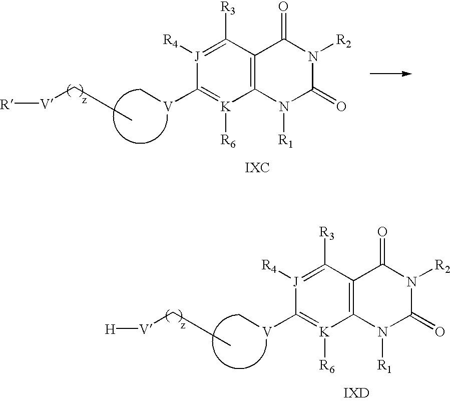

The invention also provides a process for preparing a compound of formula IX, wherein R₁, R₃, R₄, R₆, J, K, V′, z, and R′ are as defined above and R_{5′} is halo, comprising:

(a) coupling compound IXA′ with compound IXB′ in the presence of base to provide the R5-coupled product IXC′;

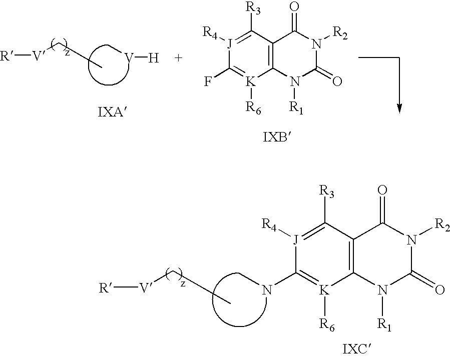

and

(b) removing the R′ group in IXC′ to provide compound IXD′

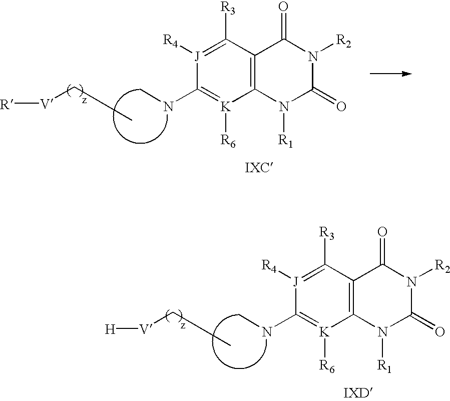

### DETAILED DESCRIPTION OF THE INVENTION

The following definitions are used, unless otherwise described: “Ph” is phenyl; halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

The term “alkyl” means a straight or branched hydrocarbon radical having from 1 to 7 carbon atoms and includes, for example, methyl, ethyl, n-propyl, isopropyl, n-butyl, sec-butyl, isobutyl, tert-butyl, n-pentyl, n-hexyl, n-heptyl, and the like.

The term “C₂-C₇ alkenyl” means a straight or branched hydrocarbon radical having from 1 to 3 double bonds. Examples include ethenyl, 2-propen-1-yl, 1,3-butadien-1-yl, 3-hexen-1-yl, 5-octen-2-yl, 2-isopropyl-3,5-octadien-1-yl, cis-3-hexen-1-yl, and trans-2-hepten-1-yl, and the like. Preferred alkenyl groups include C₂-C₆ alkenyls such as ethenyl, 2-propen-1-yl, 2-buten-1-yl, and 3-penten-1-yl, and the like.

The term “C₂-C₇ alkynyl” means a straight or branched hydrocarbon radical having from 1 to 3 triple-bonds. Examples include ethynyl, propynyl, 3-butyn-1-yl, 4-hexyn-1-yl, and 5-heptyn-3-yl, and the like. Preferred alkynyl groups are C₂-C₆ alkynyls such as ethynyl, propynyl, 3-butyn-1-yl, and 5-hexyn-1-yl, and the like.

The alkyl, alkenyl, and alkynyl groups can be substituted with one or more groups selected from halo, hydroxy, cyano, C₁-C₆ alkoxy, nitro, nitroso, amino, C₁-C₆ alkylamino, di-C₁-C₆ alkylamino, carboxy, C₁-C₆ alkoxycarbonyl, aminocarbonyl, halomethyl, dihalomethyl, trihalomethyl, haloethyl, dihaloethyl, trihaloethyl, tetrahaloethyl, pentahaloethyl, thiol, (C₁-C₄)alkylsulfanyl, (C₁-C₄)alkylsulfinyl, and aminosulfonyl, —NH—SO₂—NH₂, —O—SO₂—NH₂—, —NH—SO₂—NH₂,

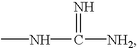

(C₁-C₆)dialkylthio, —NH—SO₂—R, where R is (C₁-C₆)alkyl, and aryl, as defined below. Examples of substituted alkyl groups include fluoromethyl, difluoromethyl, trifluoromethyl, tribromomethyl, hydroxymethyl, 3-methoxypropyl, 3-carboxypentyl, 3,5-dibromo-6-aminocarbonyldecyl, and 4-ethylsulfinyloctyl. Examples of substituted alkenyl groups include 2-bromoethenyl, 1-amino-2-propen-1-yl, 3-hydroxypent-2-en-1-yl, 4-methoxycarbonyl-hex-2-en-1-yl, and 2-nitro-3-bromo-4-iodo-oct-5-en-1-yl. Typical substituted alkynyl groups include 2-hydroxyethynyl, 3-dimethylamino-hex-5-yn-1-yl, and 2-cyano-hept-3-yn-1-yl.

The term “cycloalkyl” means a hydrocarbon ring containing from 3 to 12 carbon atoms, for example, cyclopropyl, cyclobutyl, cyclopentyl, cyclohexyl, cycloheptyl, cycloctyl, decalinyl, norpinanyl, and adamantyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, nitro, halogen, amino, alkyl and dialkylamino, formyl, carboxyl, CN, —NH—CO—R, —CO—NHR, —CO₂R, —COR, wherein R is defined as above, aryl, heteroaryl, wherein alkyl, aryl, and heteroaryl are as defined herein, or as indicated above for alkyl, alkenyl, and alkynyl substitutents. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics 10 contain from about 5 to about 17 ring atoms, preferably from 5 to 12 ring atoms.

Bicyclic heterocyclic rings may be fused, spiro, or bridged ring systems.

Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

The term “aryl” means a cyclic or polycyclic aromatic ring having from 5 to 12 carbon atoms, and being unsubstituted or substituted with one or more of the substituent groups recited above for alkyl, alkenyl, and alkynyl groups. Examples of aryl groups include phenyl, 2,6-dichlorophenyl, 3-methoxyphenyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl.

Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

A “prodrug” is an inactive derivative of a drug molecule that requires a chemical or an enzymatic biotransformation in order to release the active parent drug in the body.

“Tautomers” are structural isomersthat are conceptually related by the shift of a H or labile group (such as an acetoxy group) and one or more Π bonds. A compound of the present invention exists in two tautomeric forms, depicted below:

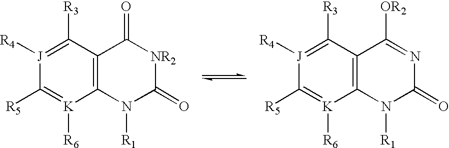

R₂═H, Ac, etc.

Specific and preferred values for compounds of Formula I are listed below for radicals, substituents, and ranges are for illustration purposes only, and they do not exclude other defined values or other values within defined ranges for the radicals and substituents.

A specific value for J is C. Another specific value for J is N.

A specific value for K is C. Another specific value for K is N.

A specific value for R₁ is methyl. Another specific value for R₁ is ethyl, isopropyl, cyclopropyl, t-butyl, 2-fluorocyclopropyl, 1- or 2-methylcyclopropyl, cyclopropylmethyl, vinyl, phenyl or substituted phenyl, heteroaryl or substituted heteroaryl.

A specific value for R₂ is H.

A specific value for each of R₃, R₄, and R₆ is H, OH, (O)ₙC₁-C₇ alkyl and substituted alkyl, (O)ₙC₂-C₇ alkenyl and substituted alkenyl, (O)ₙC₂-C₇ alkynyl and substituted alkynyl, wherein n is 0 or 1; halo, NO₂, CN, NR_{g}Rₕ, wherein R_{g} and Rₕ independently are H, C₁-C₇ alkyl and substituted alkyl, C₂-C₇ alkenyl and substituted alkenyl, C₂-C₇ alkynyl and substituted alkynyl, —CO—C₁-C₇ alkyl and substituted alkyl, or R_{g} and Rₕ taken together with the nitrogen to which they are attached form a 3- to 7-membered ring containing from 1 to 3 heteroatoms selected from N, O, and S, said ring being unsubstituted or substituted with 1, 2, 3, or 4 substituent groups.

A specific value for R₅ is C₁-C₇ alkyl and substituted alkyl, C₂-C₇ alkenyl and substituted alkenyl, C₂-C₇ alkynyl and substituted alkynyl, —CO₂Rₐ, wherein Rₐ is defined as above, —OCO₂R_{c},

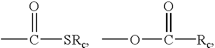

—COR_{b}, wherein R_{b} is defined as above, —(Z)ₚCONR_{c}R_{d}, wherein Z is N Or 0, p is 0 or 1, and R_{c} and R_{d} are defined as above; halo, NO₂, CN, NRᵢRⱼ, wherein Rᵢ and Rⱼ independently are H, C₁-C₇ alkyl and substituted alkyl, C₂-C₇ alkenyl and substituted alkenyl, C₂-C₇ alkynyl and substituted alkynyl, CO—C₁-C₇ alkyl and substituted alkyl, or Rᵢ and Rⱼ taken together with the nitrogen to which they are attached form a 3- to 7-membered ring containing from 1 to 3 heteroatoms selected from N, O, and S, said ring being unsubstituted or substituted with 1, 2, 3, or 4 substituent groups; aryl, fused aryl, heterocyclic, fused heterocyclic, bicyclic heterocyclic, or spiro heterocyclic, wherein fused aryl, fused heterocyclic, bicyclic heterocyclic, or spiro heterocyclic can be substituted.

Further examples of typical heterocycles, fused bicyclic or spiro heterocycles, and heteroaryl groups that are specific values for R₅ are listed below in Table 1. In Table 1, “N

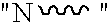

” indicates the point of attachment. It is additionally to be understood that the “N

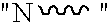

” point of attacment in the groups disclosed in the table may be replaced by a

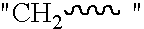

or

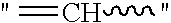

point of attachment, such as in

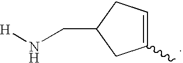

Morevoer, many of the entries depicted in Table 1 incorporate additional functionality such as primary and secondary amino groups, hydroxy groups, and thio groups. These additional functional groups can be protected by protecting groups known in the art, according to methods known in the art, as provided below.

TABLE 1

| | | | |
|---|---|---|---|
| 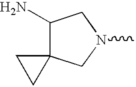 | 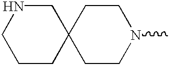 | 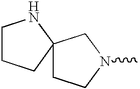 | 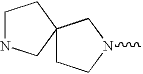 |
| 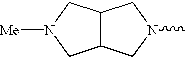 | 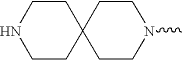 | 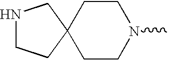 | 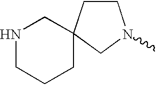 |
| 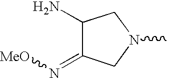 | 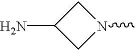 | 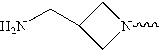 | 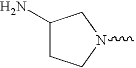 |
| 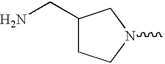 | 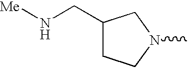 | 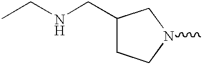 | 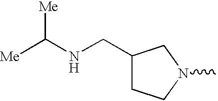 |
| 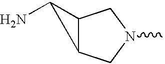 | 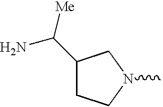 | 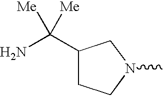 | 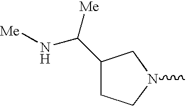 |
| 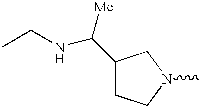 | 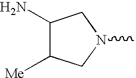 | 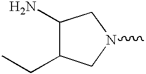 | 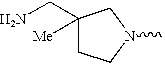 |
| 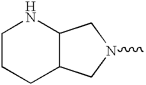 | 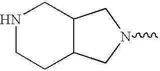 | 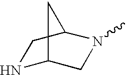 | 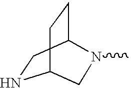 |
| 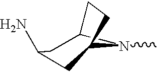 | 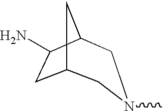 | 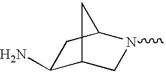 | 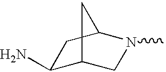 |
| 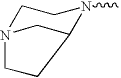 | 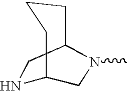 | 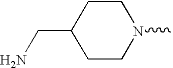 | 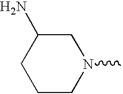 |
| 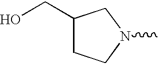 | 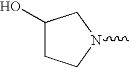 | 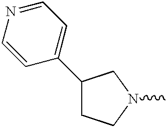 | 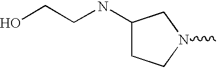 |
| 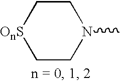 | 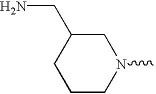 | 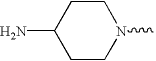 | 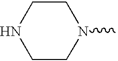 |
| 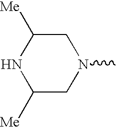 | 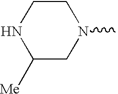 | 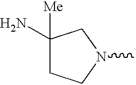 | 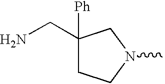 |
| 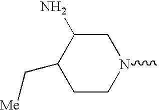 | 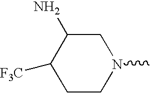 | 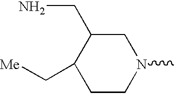 | 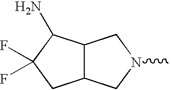 |
| 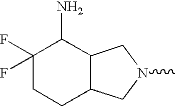 | 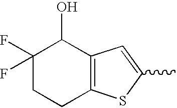 | 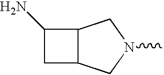 | 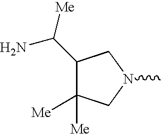 |
| 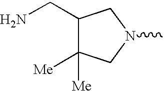 | 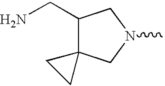 | 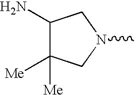 | 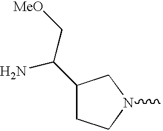 |
| 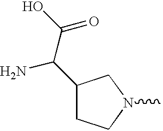 | 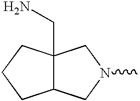 | 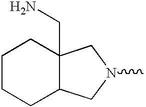 | 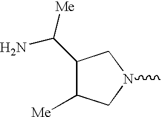 |
| 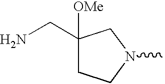 | 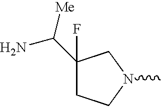 | 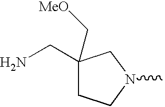 | 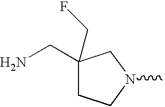 |
| 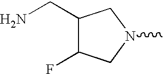 | 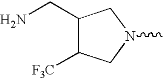 | 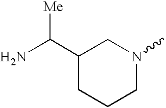 | 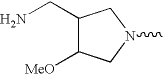 |
| 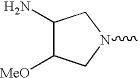 | 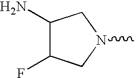 | 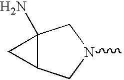 | 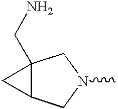 |
| 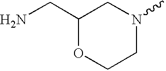 | 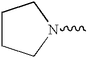 | 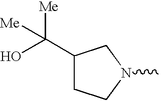 | 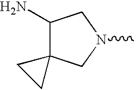 |
| 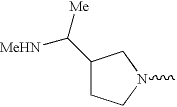 | 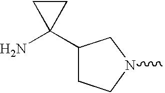 | 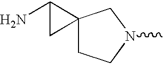 | 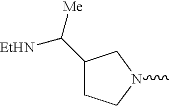 |
| 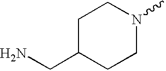 | 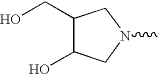 | 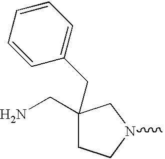 | 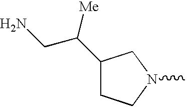 |
| 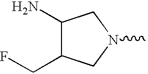 | 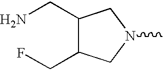 | 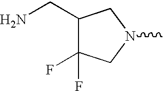 | 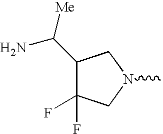 |
| 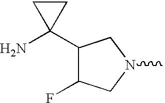 | 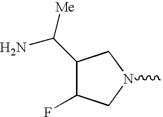 | 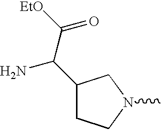 | 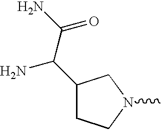 |
| 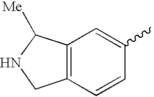 | 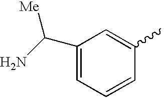 | 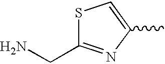 | 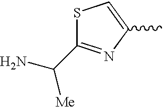 |
| 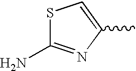 | 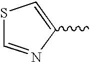 | 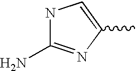 | 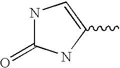 |
| 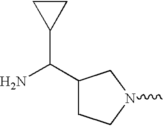 | 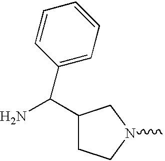 | 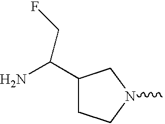 | 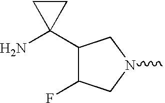 |
| 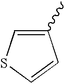 | 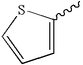 | 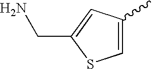 | 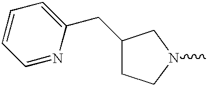 |
| 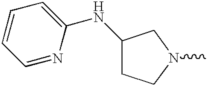 | 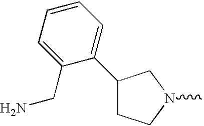 | 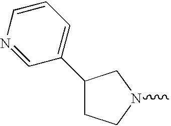 | 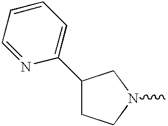 |
| 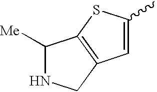 | 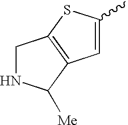 | 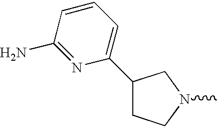 | 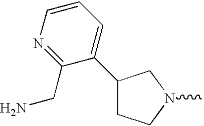 |
| 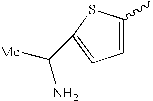 | 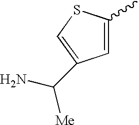 | 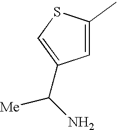 | 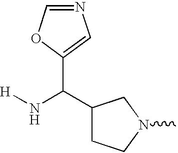 |
| 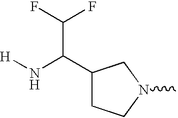 | 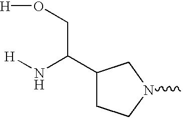 | 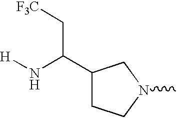 | 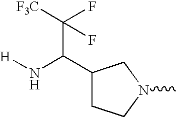 |
| 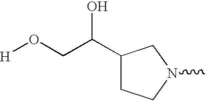 | 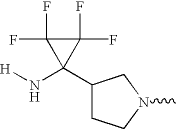 | 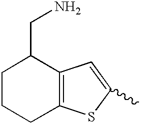 | 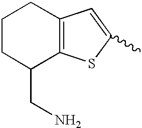 |
| 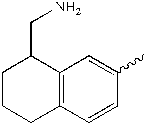 | 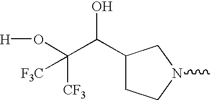 | | |

In compounds of Formula 1, a preferred value for J is C. Another preferred value for J is N. A preferred value for K is C. Another preferred value for K is N. A preferred value for R₁ is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is methyl. Another preferred value for R₃ is F. Another preferred value for R₃ is methoxy. Another preferred value for R₃ is NH₂. A preferred value for R₄ when J is C is H. A preferred value for R₄ when J is C is F. Another preferred value for R₄ when J is C is Cl. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl. Other preferred values for R₅ include heterocycles and heteroaryl groups such as those known in the quinolone art, for instance, as found in J. Med. Chem., 1992;35:1764; J. Med. Chem., 1996;39:3070; Synlett.,1996:1097; and J. Med. Chem., 1986;29:445; or, for example,

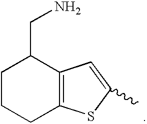

A preferred value for R₆ when K is C is H. Another preferred value for R₆ when K is C is C₁-C₄ alkyl and substituted alkyl, halo, OH, or —O—C₁-C₄ alkyl and substituted —O—C₁-C₄ alkyl, OCF₃, OCHF₂, OCH₂F, OCH₂CF₃, OCH₂CHF₂, or OCH₂CH₂F.

A preferred group of compounds of Formula I are compounds wherein J and K are C; R₁ is methyl, ethyl, cyclopropyl, t-butyl, 2-fluorocyclopropyl; R₂ is H; R₃ is H, F, Me, or NH₂; R₄ is F or Cl; R₅ is 1-pyrrolidinyl or substituted 1-pyrrolidinyl, 1-piperidinyl or substituted 1-piperidinyl, 1-piperizinyl or substituted 1-piperizinyl

or

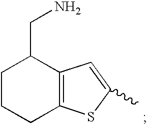

and R₆ is F, Cl, methyl, methoxy, OCF₃, OCHF₂, OCH₂F, OCH₂CF₃, OCH₂CHF₂, or OCH₂CH₂F.

Another preferred group of compounds of Formula I are compounds wherein J is N, K is C; R₁ is cyclopropyl; R₂ is H; R₃ is H, F, Me, or NH₂; R₄ is F or Cl; and R₅ is 1-pyrrolidinyl or substituted 1-pyrrolidinyl, 1-piperidinyl or substituted 1-piperidinyl, 1-piperizinyl or substituted 1-piperizinyl, or

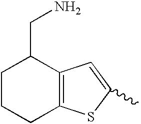

and R₆ is F, Cl, methyl, methoxy, or OCF₃.

Another preferred group of compounds of Formula I are compounds wherein J is C; K is N; R₁ is cyclopropyl; R₂ is H; R₃ is H, F, Me, or NH₂; R₄ is F or Cl; and R₅ is 1-pyrrolidinyl or substituted 1-pyrrolidinyl, 1-piperidinyl or substituted 1-piperidinyl, 1-piperizinyl or substituted 1-piperizinyl, or

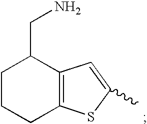

and R₆ is F, Cl, methyl, methoxy, or OCF₃.

Another preferred group of compounds of Formula I are compounds wherein J is N; K is N; R₁ is cyclopropyl; R₂ is H; R₃ is H, F, Me, or NH₂; and R₅ is 1-pyrrolidinyl or substituted 1-pyrrolidinyl, 1-piperidinyl or substituted 1-piperidinyl, 1-piperizinyl or substituted 1-piperizinyl, or

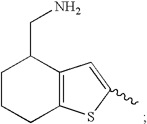

and R₆ is F, Cl, methyl, methoxy, or OCF₃.

Representative compounds of the invention which are compounds of Formula I are shown below in Table 2-I.

TABLE 2-I

| | |
|---|---|
| 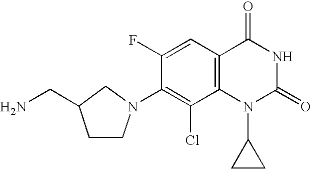 | 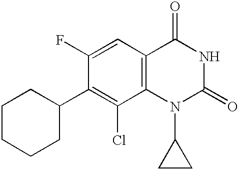 |
| 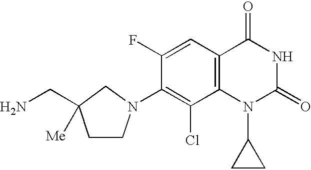 | 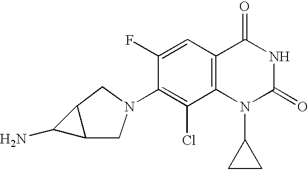 |
| 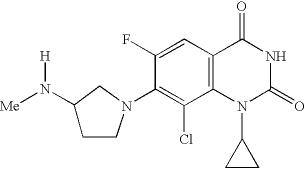 | 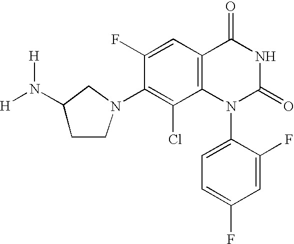 |
| 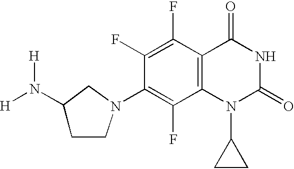 | 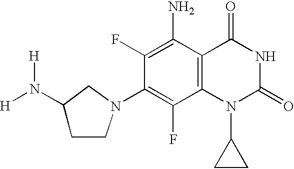 |
| 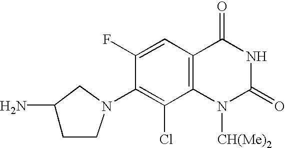 | 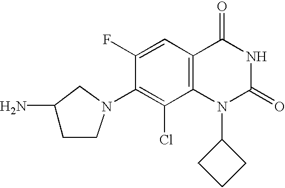 |
| 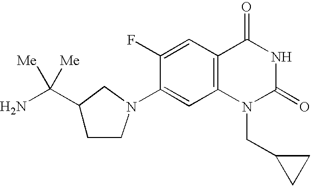 | 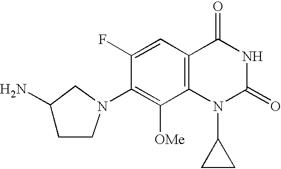 |
| 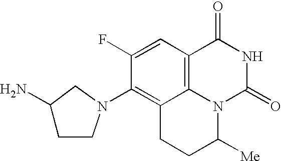 | 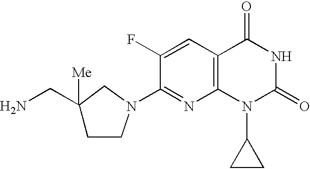 |
| 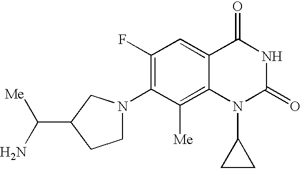 | 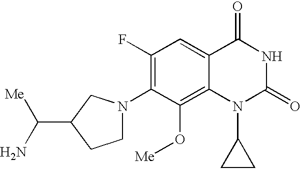 |
| 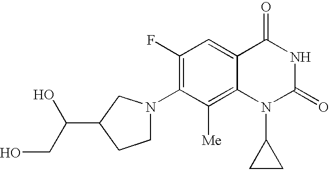 | 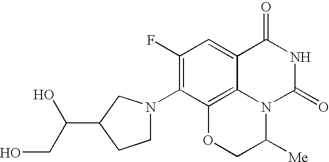 |
| 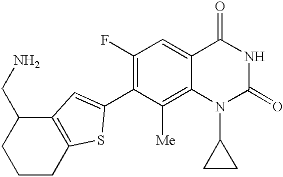 | 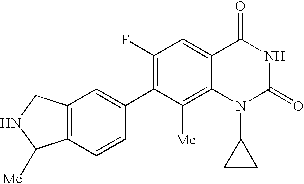 |
| 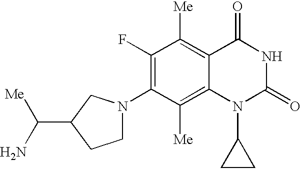 | 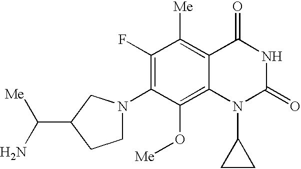 |
| 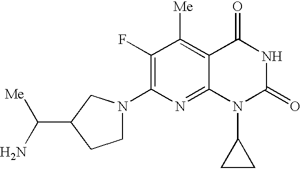 | 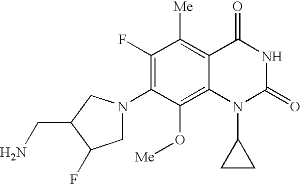 |
| 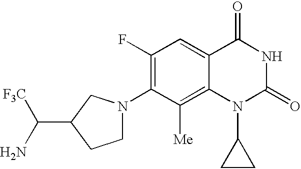 | 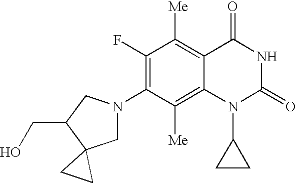 |
| 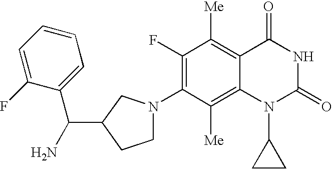 | 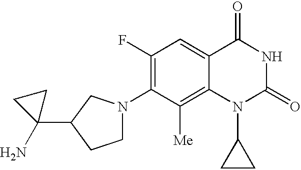 |
| 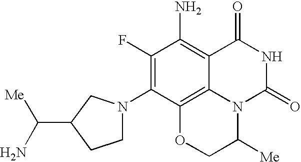 | |

Representative compounds of the present invention, which are encompassed by Formula I include, but are not limited to the compounds in Table 2 and their pharmaceutically acceptable acid or base addition salts, or amide or prodrugs thereof.

In compounds of Formula II, a preferred value for R₁ is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is F. Another preferred value for R₃ is methyl. Another preferred value for R₃ is methoxy. Another preferred value for R₃ is NH₂. A preferred value for R₄ is F. Another preferred value for R₄ is Cl. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl. Other preferred values for R₅ include heterocycles and heteroaryl groups such as those known in the quinolone art, for instance, as described above for compounds of Formula I. A preferred value for R₆ is H. Another preferred value for R₆ is C₁-C₄ alkyl and substituted alkyl, halo, OH, or —O—C₁-C₄ alkyl and substituted —O—C₁-C₄ alkyl, OCF₃, OCHF₂, OCH₂F, OCH₂CF₃, OCH₂CHF₂, or OCH₂CH₂F.

A preferred group of compounds of Formula II are compounds wherein R₁ is cyclopropyl; R₂ is H; R₃ is H, F, Me, OMe, or NH₂; R₄ is F or Cl; R₅ is 1-pyrrolidinyl or substituted 1-pyrrolidinyl, 1-piperidinyl or substituted 1-piperidinyl, 1-piperizinyl or substituted 1-piperizinyl, or

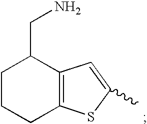

and R₆ is F; Cl, methyl, methoxy, OCF₃, OCHF₂, OCH₂F, OCH₂CF₃, OCH₂CHF₂, or OCH₂CH₂F.

Another preferred group of compounds of Formula II are compounds wherein R₁ is cyclopropyl; R₂ is H; R₃ is H, F, Me, or NH₂; R₄ is F; R₅ is 1-pyrrolidinyl or substituted 1-pyrrolidinyl, 1-piperidinyl or substituted 1-piperidinyl; 1-piperizinyl or substituted 1-piperizinyl; and R₆ is methyl, methoxy, or OCF₃.

Another preferred group of compounds of Formula II are compounds wherein R₁ is cyclopropyl; R₂ is H; R₃ is H, F, Me, or NH₂; R₄ is F; R₅ is 1-pyrrolidinyl or substituted 1-pyrrolidinyl, or 1-piperidinyl or substituted 1-piperidinyl; and R₆ is F, Cl, methyl, methoxy, or OCF₃.

Another preferred group of compounds of Formula II are compounds wherein R₁ is cyclopropyl; R₂ is H; R₃ is H, F, Me, or NH₂; R₄ is F; R₅ is 1-pyrrolidinyl; and R₆ is F, Cl, methyl, methoxy, or OCF₃.

Representative compounds of the invention which are compounds of Formula 1 are also depicted in Table 2-I.

In compounds of Formula III, a preferred value for R₁ is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is F. Another preferred value for R₃ is methyl. Another preferred value for R₃ is methoxy. Another preferred value for R₃ is NH₂. A preferred value for R₄ is F. Another preferred value for R₄ is Cl. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl, or

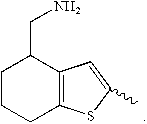

Other preferred values for R₅ include heterocycles and heteroaryl groups such as those known in the quinolone art, for instance, as described above for compounds of Formula I.

A preferred group of compounds of Formula III are compounds wherein R₁ is cyclopropyl; R₃ is H, F, Me, or NH₂; R₄ is F or Cl; and R₅ is 1-pyrrolidinyl or substituted 1-pyrrolidinyl, 1-piperidinyl or substituted 1-piperidinyl, 1-piperizinyl or substituted 1-piperizinyl, or

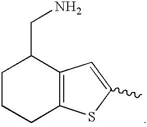

Another preferred group of compounds of Formula III are compounds wherein R₁ is cyclopropyl; R₂ is H; R₃ is H, F, Me, OMe, or NH₂; R₄ is F; and R₅ is 1-pyrrolidinyl or substituted 1-pyrrolidinyl, 1-piperidinyl or substituted 1-piperidinyl, 1-piperizinyl or substituted 1-piperizinyl.

Another preferred group of compounds of Formula III are compounds wherein R₁ is cyclopropyl; R₂ is H; R₃ is H, F, Me, or NH₂; R₄ is F; and R₅ is 1-pyrrolidinyl or substituted 1-pyrrolidinyl, or 1-piperidinyl or substituted 1-piperidinyl.

Another preferred group of compounds of Formula III are compounds wherein R₁ is cyclopropyl; R₂ is H; R₃ is H, F, Me, or NH₂; R₄ is F; and R₅ is 1-pyrrolidinyl or substituted 1-pyrrolidinyl.

Representative compounds of the invention which are compounds of Formula III are shown below in Table 2-III.

TABLE 2-III

| | |
|---|---|
| 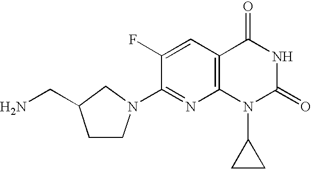 | 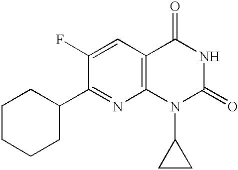 |
| 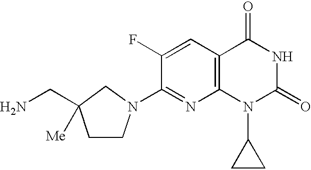 | 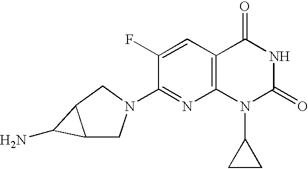 |
| 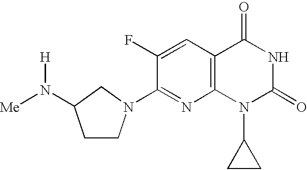 | 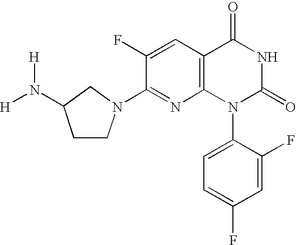 |
| 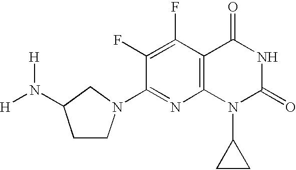 | 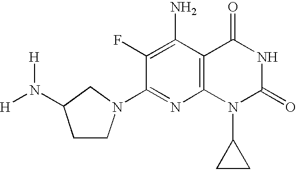 |
| 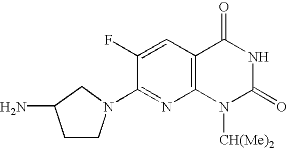 | 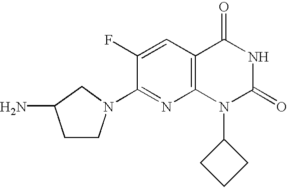 |
| 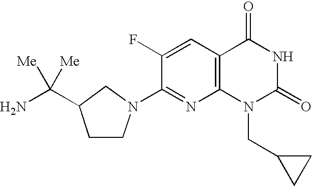 | 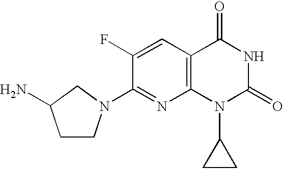 |
| 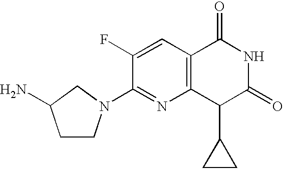 | 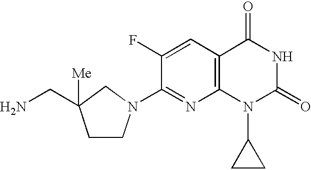 |
| 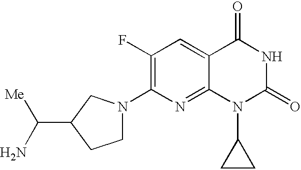 | 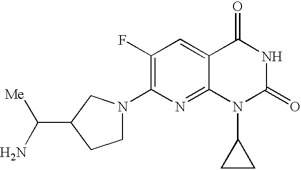 |
| 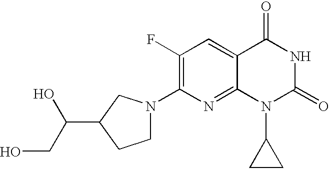 | 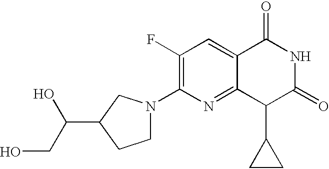 |
| 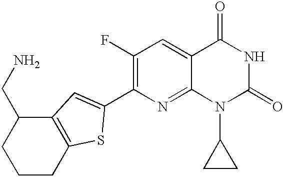 | 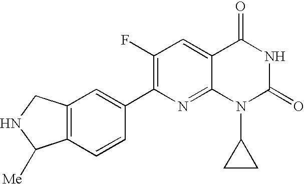 |
| 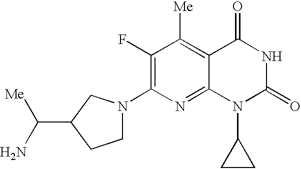 | 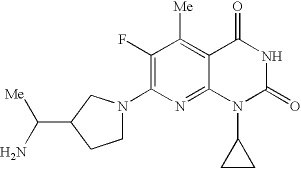 |
| 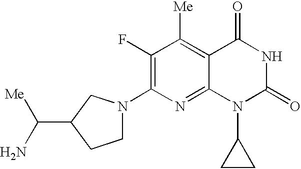 | 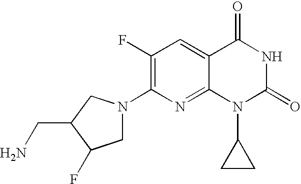 |
| 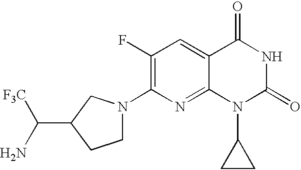 | 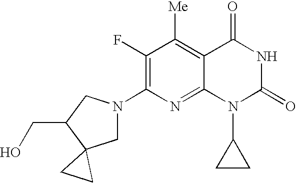 |
| 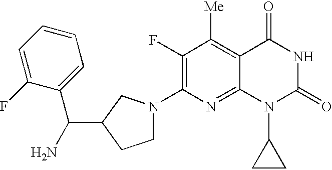 | 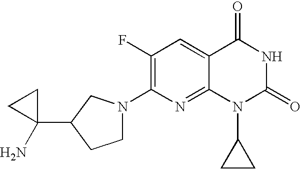 |
| 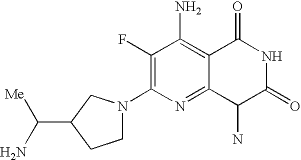 | |

Representative compounds of the present invention, which are encompassed by Formula III include, but are not limited to the compounds in Table 2-III and their pharmaceutically acceptable acid or base addition salts, or amide or prodrugs thereof.

In compounds of Formula IV, a preferred value for R₁ is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is F. Another preferred value for R₃ is methyl. Another preferred value for R₃ is methoxy. Another preferred value for R₃ is NH₂. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl, or

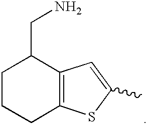

Other preferred values for R₅ include heterocycles and heteroaryl groups such as those known in the quinolone art, for instance, as described above for compounds of Formula I. A preferred value for R₆ is H. Another preferred value for R₆ is C₁-C₄ alkyl and substituted alkyl, halo, OH, or —O—C₁-C₄ alkyl and substituted —O—C₁-C₄ alkyl, OCF₃, OCHF₂, OCH₂F, OCH₂CF₃, OCH₂CHF₂, or OCH₂CH₂F.

A preferred group of compounds of Formula IV are compounds wherein R₁ is cyclopropyl; R₂ is H; R₃ is H, methyl, NH₂, or methoxy; R₅ is 1-pyrrolidinyl or substituted 1-pyrrolidinyl, 1-piperidinyl or substituted 1-piperidinyl, 1-piperizinyl or substituted 1-piperizinyl, or

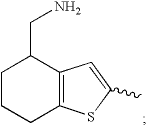

and R₆ is F; Cl, methyl, methoxy, OCF₃, OCHF₂, OCH₂F, OCH₂CF₃, OCH₂CHF₂, or OCH₂CH₂FI

Another preferred group of compounds of Formula IV are compounds wherein R₁ is cyclopropyl; R₂ is H; R₃ is H, methyl, or methoxy; R₅ is 1-pyrrolidinyl or substituted 1-pyrrolidinyl, 1-piperidinyl or substituted 1-piperidinyl, 1-piperizinyl or substituted 1-piperizinyl; and R₆ is F, Cl, methyl, methoxy, or OCF₃.

Another preferred group of compounds of Formula IV are compounds wherein R₁ is cyclopropyl; R₂ is H; R₃ is H; R₅ is 1-pyrrolidinyl or substituted 1-pyrrolidinyl, or 1-piperidinyl or substituted 1-piperidinyl; and R₆ is F, Cl, methyl, methoxy, or OCF₃.

Another preferred group of compounds of Formula IV are compounds wherein R₁ is cyclopropyl; R₂ is H; R₃ is H; R₅ is 1-pyrrolidinyl or substituted 1-pyrrolidinyl, or 1-piperidinyl or substituted 1-piperidinyl; and R₆ is F, Cl, methyl, methoxy, or OCF₃.

Representative compounds of the invention which are compounds of Formula 1 are shown below in Table 2-IV.

TABLE 2-IV

| | |
|---|---|
| 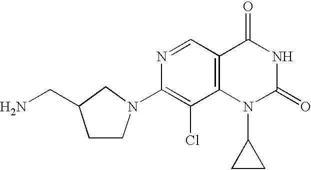 | 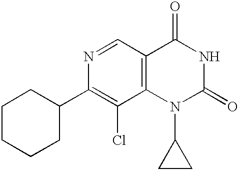 |
| 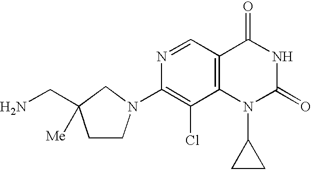 | 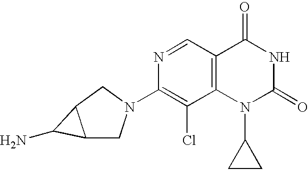 |
| 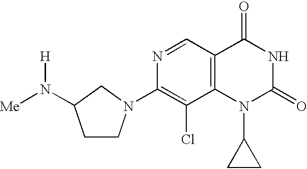 | 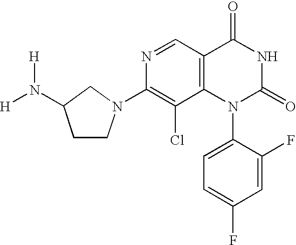 |
| 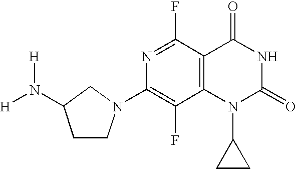 | 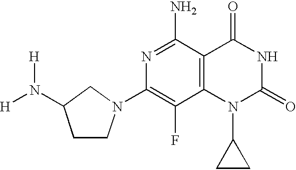 |
| 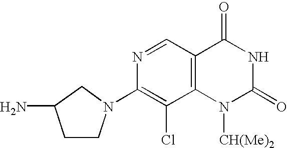 | 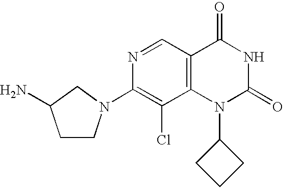 |
| 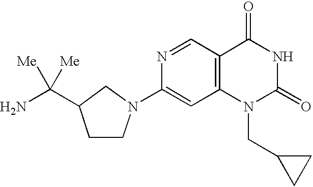 | 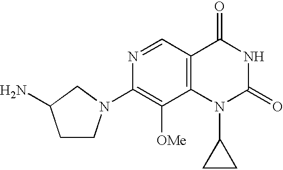 |
| 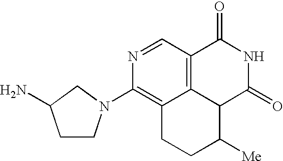 | 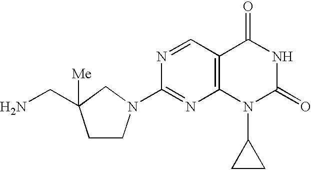 |
| 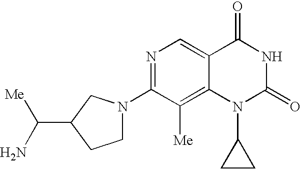 | 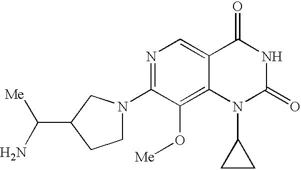 |
| 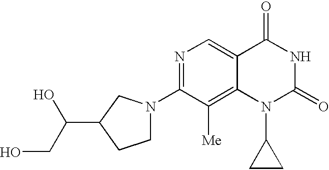 | 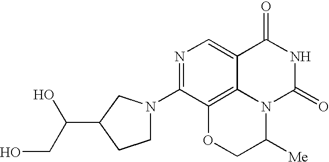 |
| 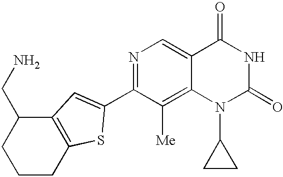 | 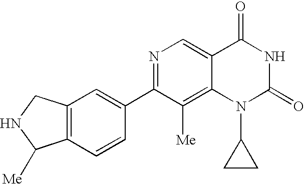 |
| 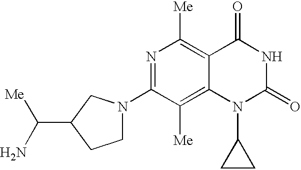 | 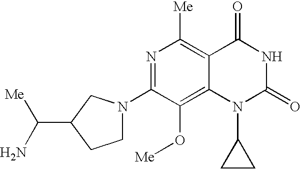 |
| 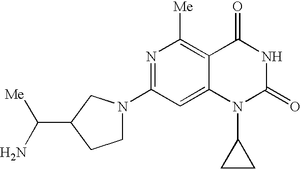 | 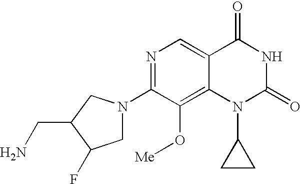 |
| 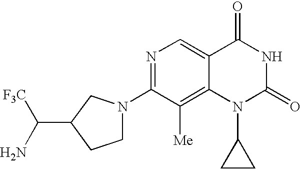 | 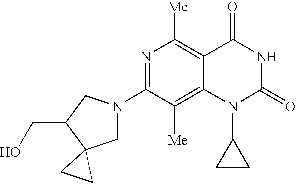 |
| 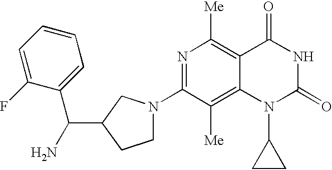 | 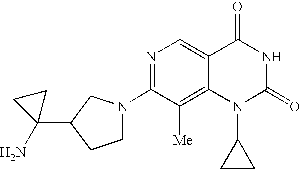 |
| 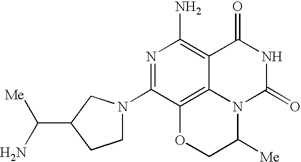 | |

Representative compounds of the present invention, which are encompassed by Formula IV include, but are not limited to the compounds in Table 2-IV and their pharmaceutically acceptable acid or base addition salts, or amide or prodrugs thereof.

In compounds of Formula V, a preferred value for R₁ is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is F. Another preferred value for R₃ is methyl. Another preferred value for R₃ is methoxy. Another preferred value for R₃ is NH₂. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl, or

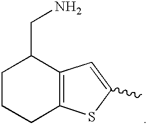

Other preferred values for R₅ include heterocycles and heteroaryl groups such as those known in the quinolone art, for instance, as described above for compounds of Formula I.

A preferred group of compounds of Formula V are compounds wherein R₁ is cyclopropyl; R₂ is H; R₃ is H, methyl, NH₂, or methoxy; and R₅ is 1-pyrrolidinyl or substituted 1-pyrrolidinyl, 1-piperidinyl or substituted 1-piperidinyl, 1-piperizinyl or substituted 1-piperizinyl, or

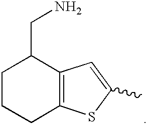

Another preferred group of compounds of Formula V are compounds wherein R₁ is cyclopropyl; R₂ is H; R₃ is H, methyl, NH₂, or methoxy; and R₅ is 1-pyrrolidinyl or substituted 1-pyrrolidinyl, or 1-piperidinyl or substituted 1-piperidinyl.

Another preferred group of compounds of Formula V are compounds wherein R₁ is cyclopropyl; R₂ is H; R₃ is H, methyl, NH₂, or methoxy; and R₅ is 1-pyrrolidinyl or substituted 1-pyrrolidinyl.

Another preferred group of compounds of Formula V are compounds wherein R₁ is cyclopropyl; R₃ is H, methyl, NH₂, or methoxy; and R₅ is substituted 1-pyrrolidinyl.

Representative compounds of the invention which are compounds of Formula V are shown below in Table 2-V.

TABLE 2-V

| | |
|---|---|
| 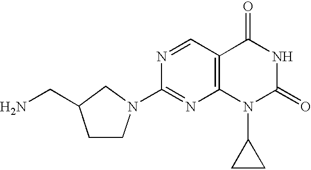 | 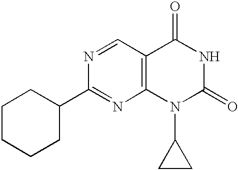 |
| 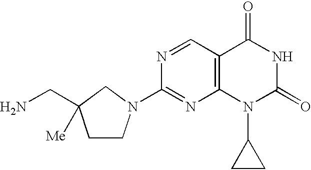 | 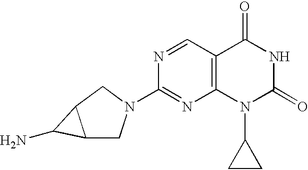 |
| 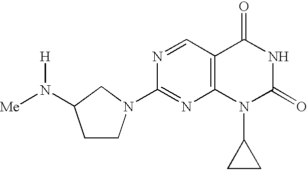 | 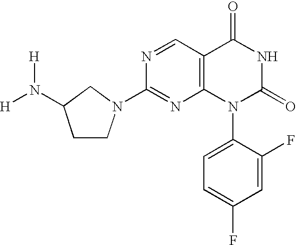 |
| 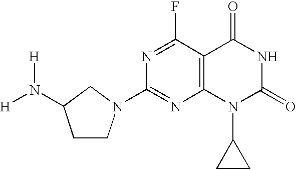 | 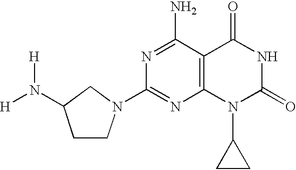 |
| 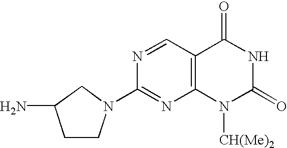 | 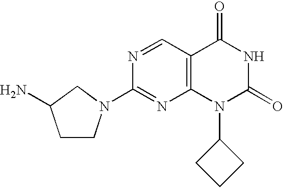 |
| 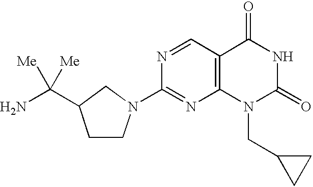 | 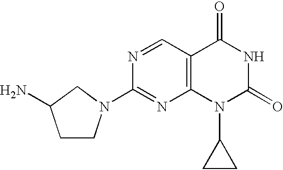 |
| 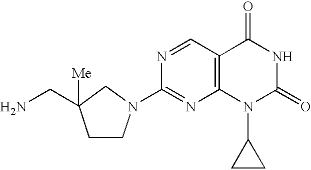 | 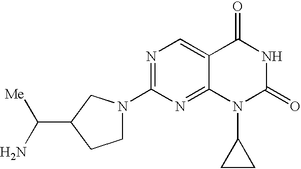 |
| 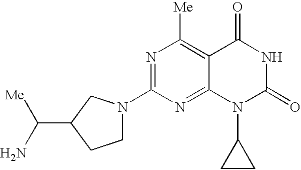 | 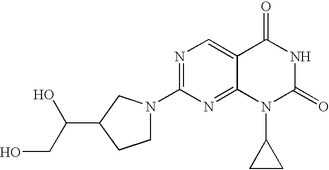 |
| 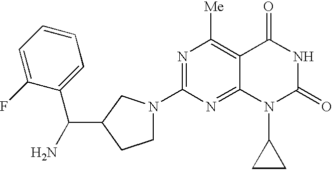 | 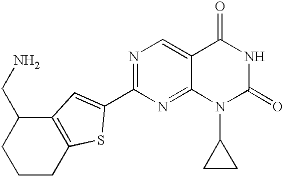 |
| 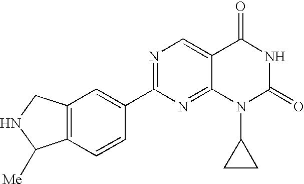 | 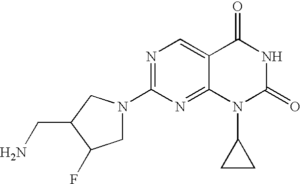 |
| 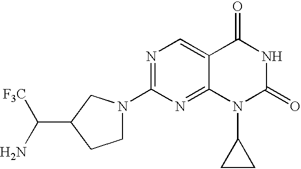 | 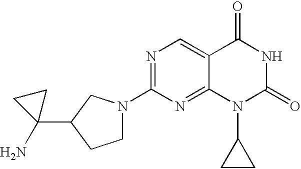 |
| 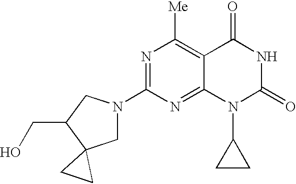 | |

Representative compounds of the present invention, which are encompassed by Formula V include, but are not limited to the compounds in Table 2-V and their pharmaceutically acceptable acid or base addition salts, or amide or prodrugs thereof.

In compounds of Formula VI, a preferred value for Rn is cyclopropyl. Another preferred value for R₁ is 2-fluorocyclopropyl, 1- or 2-methylcyclopropyl, or cyclopropylmethyl. A preferred value for R₂ is H. A preferred value for R₃ is H. Another preferred value for R₃ is methyl. Another preferred value for R₃ is methoxy. Another preferred value for R₃ is NH₂. A preferred value for R₄ is F. Another preferred value for R₄ is Cl. A preferred value for R_{f} and R_{g}, together with the nitrogen to which they are attached, is 1-pyrrolidinyl or substituted 1-pyrrolidinyl. Another preferred value for R_{f} and R_{g}, together with the nitrogen to which they are attached, is 1-piperidinyl or substituted 1-piperidinyl, or 1-piperizinyl or substituted 1-piperizinyl. Other preferred values for R_{f} and R_{g}, together with the nitrogen to which they are attached, include heterocycles and heteroaryl groups such as those known in the quinolone art, for instance, as described above for compounds of Formula I. A preferred value for R₆ is H. Another preferred value for R₆ is C₁-C₄ alkyl and substituted alkyl, halo, OH, or —O—C₁-C₄ alkyl and substituted —O—C₁-C₄ alkyl, OCF₃, OCHF₂, OCH₂F, OCH₂CF₃, OCH₂CHF₂, or OCH₂CH₂F.

A preferred group of compounds of Formula VI are compounds wherein R₁ is cyclopropyl; R₂ is H; R₃ is H, methyl, NH₂, or methoxy; R₄ is F or Cl; R_{f} and R_{g}, together with the nitrogen to which they are attached, are 1-pyrrolidinyl or substituted 1-pyrrolidinyl, 1-piperidinyl or substituted 1-piperidinyl, 1-piperizinyl or substituted 1-piperizinyl; and R₆ is F, Cl, methyl, methoxy, OCF₃, OCHF₂, OCH₂F, OCH₂CF₃, OCH₂CHF₂, or OCH₂CH₂F.

Another preferred group of compounds of Formula VI are compounds wherein R₁ is cyclopropyl; R₂ is H; R₃ is H, methyl, NH₂, or methoxy; R₄ is F; R_{f} and R_{g}, together with the nitrogen to which they are attached, are 1-pyrrolidinyl or substituted 1-pyrrolidinyl, 1-piperidinyl or substituted 1-piperidinyl, 1-piperizinyl or substituted 1-piperizinyl; and R₆ is methyl, methoxy, OCF₃, OCHF₂, OCH₂F, or OCH₂CF₃.

Another preferred group of compounds of Formula VI are compounds wherein R₁ is cyclopropyl; R₂ is H; R₃ is H, methyl, NH₂, or methoxy; R₄ is F; R_{f} and R_{g}, together with the nitrogen to which they are attached, are 1-pyrrolidinyl or substituted 1-pyrrolidinyl, or 1-piperidinyl or substituted 1-piperidinyl; and R₆ is methyl, methoxy, or OCF₃.

Another preferred group of compounds of Formula VI are compounds wherein R₁ is cyclopropyl; R₂ is H; R₃ is H, methyl, NH₂, or methoxy; R₄ is F; R_{f} and R_{g}, together with the nitrogen to which they are attached, are 1-pyrrolidinyl or substituted 1-pyrrolidinyl; and R₆ is F, Cl, methyl, methoxy, or OCF₃.

Representative compounds of the invention which are compounds of Formula VI are also shown in Table 2-I.

In compounds of Formula VII, a preferred value for R₁ is cyclopropyl. Another preferred value for R₁ is 2-fluorocyclopropyl, 1- or 2-methylcyclopropyl, or cyclopropylmethyl. A preferred value for R₃ is H. Another preferred value for R₃ is methyl. Another preferred value for R₃ is methoxy. Another preferred value for R₃ is NH₂. A preferred value for R₄ is F. Another preferred value for R₄ is Cl. A preferred value for R_{g} and Rₕ, together with the nitrogen to which they are attached, is 1-pyrrolidinyl or substituted 1-pyrrolidinyl. Another preferred value for R_{g} and Rₕ, together with the nitrogen to which they are attached, is 1-piperidinyl or substituted 1-piperidinyl, or 1-piperizinyl or substituted 1-piperizinyl. Other preferred values for R_{g} and Rₕ, together with the nitrogen to which they are attached, include heterocycles and heteroaryl groups such as those known in the quinolone art, for instance, as described above for compounds of Formula I.

A preferred group of compounds of Formula VII are compounds wherein R₁ is cyclopropyl; R₃ is H, methyl, NH₂, or methoxy; R₄ is F or Cl; and R_{g} and Rₕ, together with the nitrogen to which they are attached, are 1-pyrrolidinyl or substituted 1-pyrrolidinyl, 1-piperidinyl or substituted 1-piperidinyl, 1-piperizinyl or substituted 1-piperizinyl.

Another preferred group of compounds of Formula VII are compounds wherein R₁ is cyclopropyl; R₃ is H, methyl, NH₂, or methoxy; R₄ is F; and R_{g} and Rₕ, together with the nitrogen to which they are attached, are 1-pyrrolidinyl or substituted 1-pyrrolidinyl, or 1-piperidinyl or substituted 1-piperidinyl.

Another preferred group of compounds of Formula VII are compounds wherein R₁ is cyclopropyl; R₃ is H, methyl, NH₂, or methoxy; R₄ is F; and R_{g} and Rₕ, together with the nitrogen to which they are attached, are 1-pyrrolidinyl or substituted 1-pyrrolidinyl.

Another preferred group of compounds of Formula VII are compounds wherein R₁ is cyclopropyl; R₃ is H, methyl, NH₂, or methoxy; R₄ is F; R_{g} and Rₕ, together with the nitrogen to which they are attached, are substituted 1-pyrrolidinyl.

Representative compounds of the invention which are compounds of Formula VII are shown in Table 2-III.

In compounds of Formula VIII, a preferred value for m is 0 or 1. A preferred value for X is O. Another preferred value for X is CH₂ or CH(C₁-C₇ alkyl). A preferred value for Y is CH₂ or CH(C₁-C₇ alkyl). Another preferred value for Y is C(C₁-C₇ alkyl)₂, C(C₃-C₆ cycloalkyl),

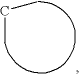

wherein

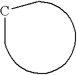

is a C₃-C₆ cycloalkyl. Another preferred value for Y is NH or N(C₁-C₇ alkyl). Another preferred value for Y is C(C₁-C₇ alkyl)₂, C(C₃-C₆ cycloalkyl),

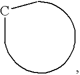

wherein

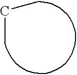

is a C₃-C₆ cycloalkyl. A preferred value for Rⱼ is C₁-C₇ alkyl. A preferred value for R₂ is H. A preferred value for R₃ is H. Another preferred value for R₃ is methyl. Another preferred value for R₃ is methoxy. Another preferred value for R₃ is NH₂. A preferred value for R₄ is F. Another preferred value for R₄ is Cl. A preferred value for R₅ is 1-pyrrolidinyl or substituted 1-pyrrolidinyl. Another preferred value for R₅ is 1-piperidinyl or substituted 1-piperidinyl, or 1-piperizinyl or substituted 1-piperizinyl, or

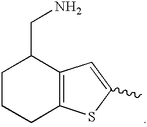

Other preferred values for R₅ include heterocycles and heteroaryl groups such as those known in the quinolone art, for instance, as described above for compounds of Formula I.

A preferred group of compounds of Formula VIII are compounds wherein m is 1; X is O or CH₂; Y is CH₂, CH(C₁-C₇ alkyl), NH, or N(C₁-C₇ alkyl); Rₕ is methyl; R₂ is H; R₃ is H; R₄ is F or Cl; and R₅ is 1-pyrrolidinyl or substituted 1-pyrrolidinyl, 1-piperidinyl or substituted 1-piperidinyl, 1-piperizinyl or substituted 1-piperizinyl.

Another preferred group of compounds of Formula VIII are compounds wherein X is O or CH₂; Y is CH₂, CH(C₁-C₇ alkyl), NH, or N(C₁-C₇ alky l), Rₕ is methyl; R₂ is H; R₃ is H; and R₅ is 1-pyrrolidinyl or substituted 1-pyrrolidinyl, or 1-piperidinyl or substituted 1-piperidinyl.

Another preferred group of compounds of Formula VIII are compounds wherein X is O or CH₂; Y is CH₂, CH(C₁-C₇ alkyl), NH, or N(C₁-C₇ alkyl); Rₕ is methyl; R₂ is H; R₃ is H; R₄ is F or Cl; and R₅ is 1-pyrrolidinyl or substituted 1-pyrrolidinyl.

Another preferred group of compounds of Formula VIII are compounds wherein X is O or CH₂; Y is CH₂, CH(C₁-C₇ alkyl), NH, or N(C₁-C₇ alkyl); Rₕ is methyl; R₂ is H; R₃ is H; and R₅ is substituted 1-pyrrolidinyl.

Representative compounds of the invention which are compounds of Formula VIII are shown below in Table 2-VIII.

TABLE 2-VIII

| | |
|---|---|
| 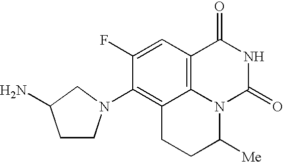 | 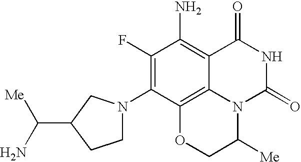 |

Representative compounds of the present invention, which are encompassed by Formula 2-VIII include, but are not limited to the compounds in Table 2-VIII and their pharmaceutically acceptable acid or base addition salts, or amide or prodrugs thereof.

Illustrations of typical preparations of compounds of the present invention having Formula I are shown in the following synthetic schemes. Typical heterocyclic and aromatic side chains defined by R₅ in Formula I are prepared as are also described. All of the 3-hydridoquinazolinediones of the invention may be prepared from appropriately substituted benzoic acid starting materials. Protecting groups (referred to in the schemes as “Pro”) may be used when appropriate throughout many of the schemes. Although specifically noted in certain schemes, the appropriate use and choice of protecting groups is well-known by those skilled in the art, and is not limited to the specific illustrations shown below. It should also be understood that such protecting groups not only serve to protect chemically reactive sites, but also to enhance solubility or otherwise change physical properties of the underlying invention compound. A number of general reactions such as oxidations and reductions are not shown in detail in the schemes, but can be carried out by standard methods well-known to those skilled in the art. In general, the starting materials used in the following schemes are obtained from commercial sources, or are readily prepared by standard methods. All cited published articles, patents, books, and the like are incorporated herein by reference.

The following Schemes are organized in two parts. The first part summarizes synthetic approaches to the core structures represented by compounds of Formulas I-VIII, depicted generally by the reference structure in the following drawing. The second part summarizes synthetic approaches to the preparation of particular R₅ groups that can be attached to the core structures. The meaning of “core structure” and “sidechain” is demonstrated in the following drawing by structure X. As will be seen below, there are two general approaches to preparing compounds of the present invention. In the first general approach, the core structure is prepared and then the R₅ sidechain is attached to the core structure to provide a compound of the invention. In the second general approach, the R₅ sidechain is attached to a precursor of the core structure and then the core structure is prepared by intramolecular cyclization. Both approaches are disclosed in the following section.

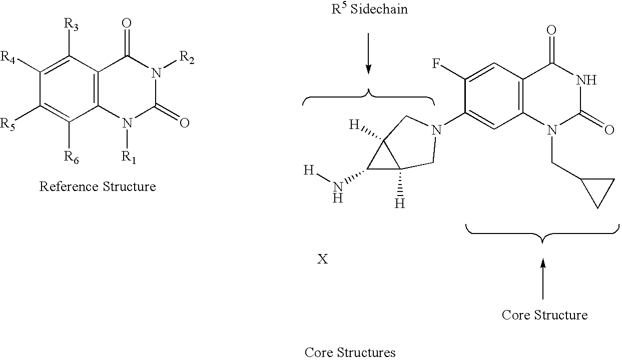

The core structures of the compounds of the invention can be prepared from appropriately substituted benzoic acid derivatives. Thus compounds of formula I wherein J is C or N, K is C, and R₆ is C₁-C₇ alkyl or substituted alkyl can be prepared as summarized in Scheme 1, wherein the R₁ and R₅ sidechain are attached subsequent to cyclization to form the dione ring. Thus, the anthranilic acid derivative can be converted to the corresponding alkoxy benzamide ester. The amide can be treated with a carbon monoxide equivalent such as phosgene to provide the 3-N-methoxy quinazolinedione derivative. Deprotonation of the position 1 amide moiety in the dione, followed by alkylation using an alkylating agent such as R₁X, wherein R₁ has any of the meanings provided herein, can give rise to the R₁ alkylated quinazolinedione derivative. An R₅ sidechain can be attached to quinazolinedione core structure using base or a palladium catalyst according to methods available to the skilled artisan. Finally, the 3-methoxy group can be removed by hydrogenation to provide the 3-hydridoquinazolinedione compound.

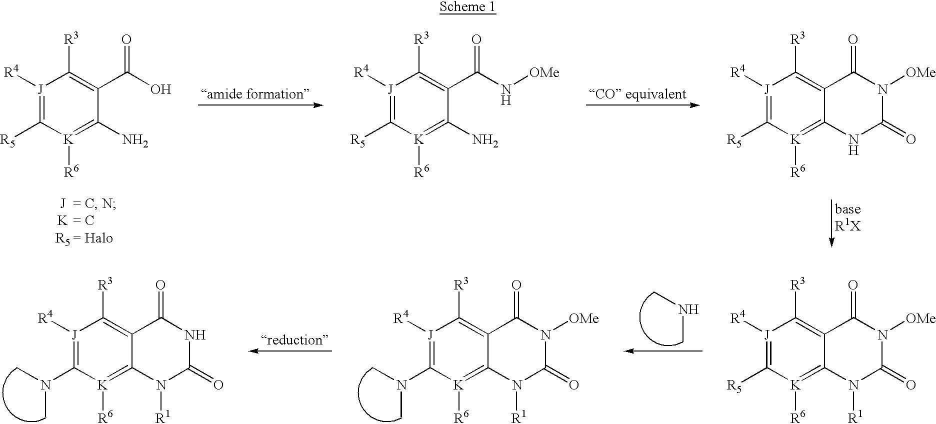

Scheme 2 provides a particular example of this approach. Thus, upon treatment with carbonyl diimidazole and O-methylhydroxy amine, 4,5-difluoroanthranilic acid can be converted to the N-methoxy benzamide derivative. Upon treatment with phosgene, the N-methoxy benzamide derivative undergoes cyclization to provide methoxyquinazolinedione. Alkylation of at the 1-position using bromomethylcyclopropane in the presence of base affords the R₁ substituted compound. Coupling of an amine at the R₅ position provides the R₅ coupled compound, which can be converted to the 3-hydrido compound under hydrogenation conditions.

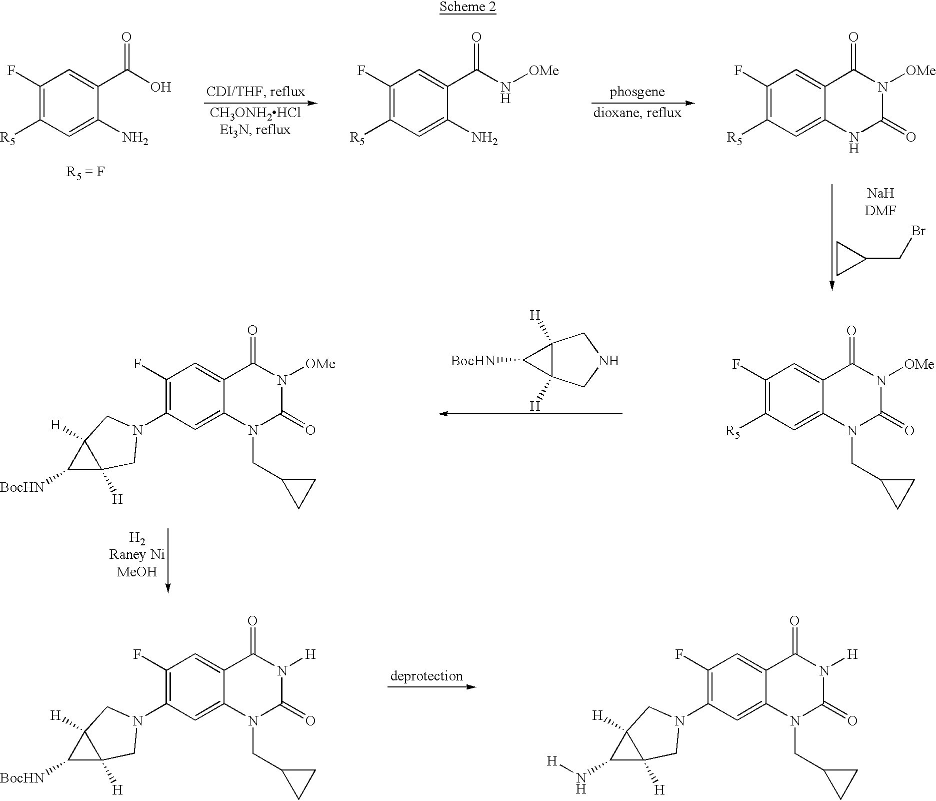

Scheme 2-A provides an alternative approach to quinazolinedione ring formation. Urea formation at the nitrogen of the cyclopropylamine moiety in the starting compound can be effected using chlorosulfonyl isocyanate. Heating of the urea in refluxing toluene, followed by removal of the protecting group, gives the invention compound.

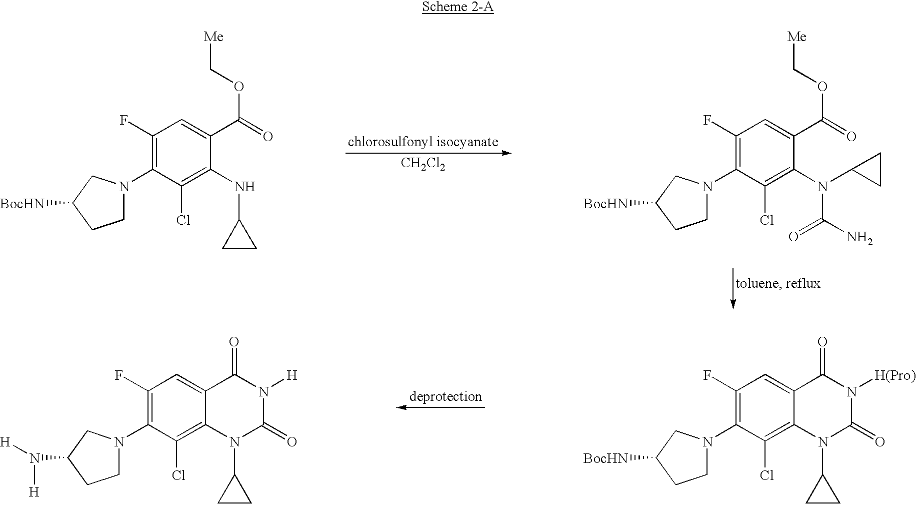

A characteristic of the approach outlined in Schemes 1, 2, and 2-A is the generation of the 3-hydrido group subsequent to the introduction of the R₅ sidechain. Scheme 3 provides an alternative approach to compounds of the invention wherein J is N or C, and wherein the R₅ sidechain is attached subsequent to the generation of 3-hydrido group. Thus, the nicotinic acid derivative can be converted to the nicotinamide derivative via an acid chloride or acid anhydride intermediate. The acid chloride or anhydride intermediate can be converted to a urea deriviative upon treatment with oxalyl chloride or an equivalent, followed by addition of R₁NH₂, wherein R₁ has any of the meanings provided herein. Cyclization of the urea can occur upon treatment of the compound with base in the presence or absence of a chelating agent, to provide a quinazolinedione. An R₅ sidechain can be attached to the dione as provided in Scheme 2.

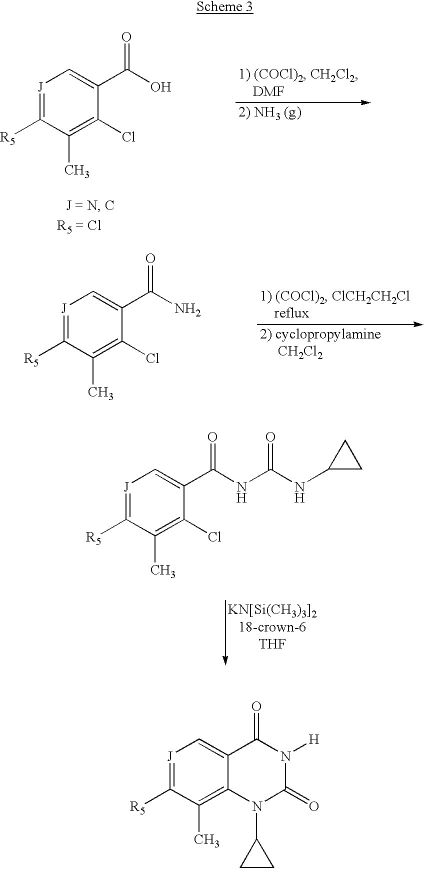

Scheme 4 provides a particular approach to invention compounds wherein R₅ is aryl. Thus, the acid is treated with acid and methanol to provide the methyl ester. Conversion to the R₅ aminated compound occurs using using benzyl amine in the presence of triethylamine and DMSO. Removal of the benzyl group using Pd/C provides the R₅ primary amine. The ester moiety is then saponified to the acid, and the R₅ amine is converted to a bromide using CuBr and t-BuNO₂. Conversion of the acid moiety to an amide, followed by generation of the acyl isocyanate and treatment with cyclopropylamine (R₁NH₂) provides the urea, which is treated with base as provided in Scheme 2-A to give a quinazolinedione with an R₅ bromo group. An aryl group can be coupled at the R₅-position using an aryl stannane in the presence of a palladium catalyst under conditions known to those skilled in the art to provide a compound of the invention.

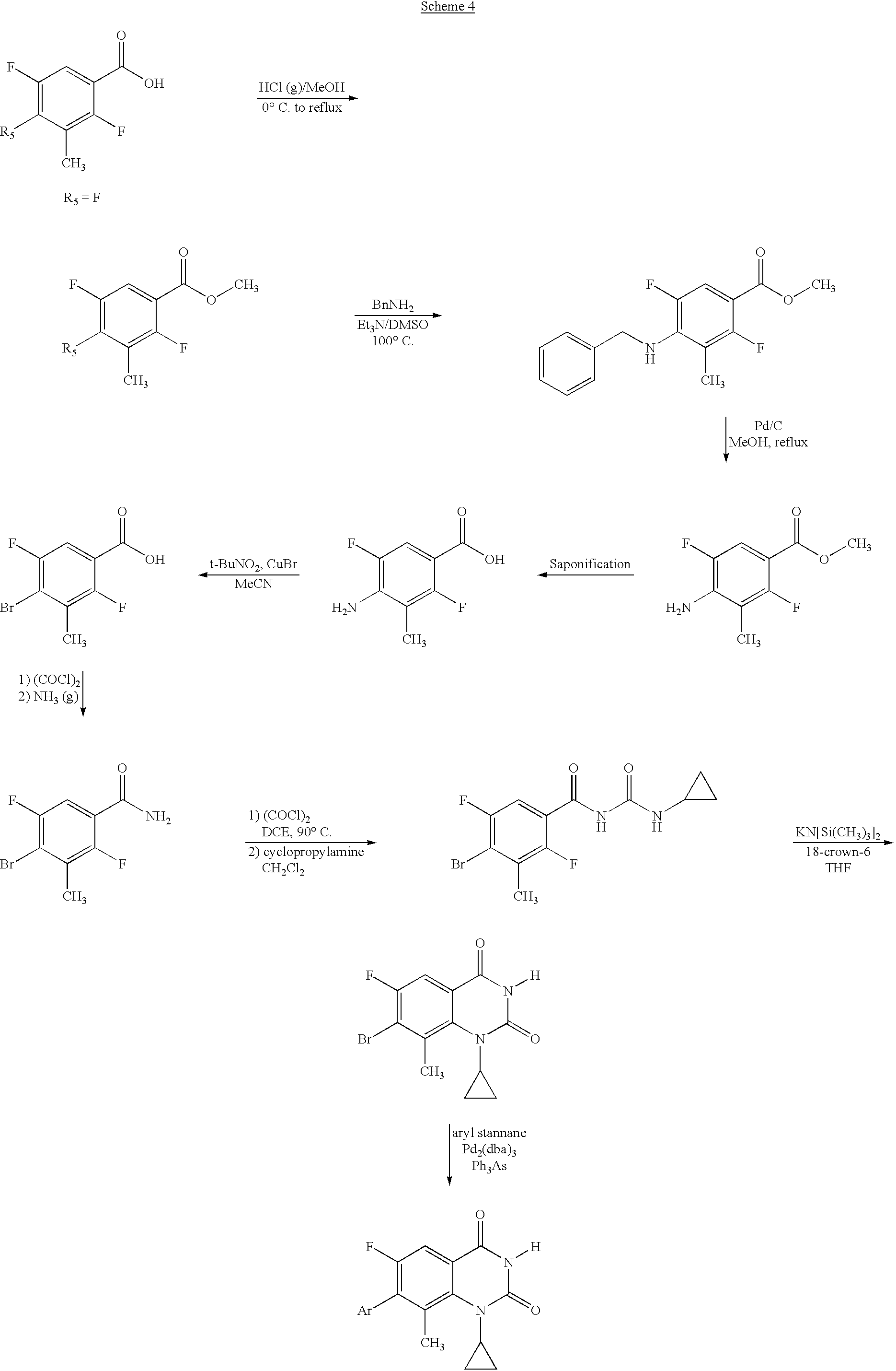

Scheme 5 provides another variation of this approach wherein R₅ is iodo. Attachment of an iodo group at the R₅-position can be effected via diazotization of the R₅ amine in the starting compound using isoamyl nitrite in the presence of CuI. Saponification of the ester moiety, followed by the series of transformations provided earlier can provide the R₅ iodo quinazolinedione compound. Attachment of an aryl, alkyl, or heterocycloalkyl group to the R₅-position of the compound can be achieved using procedures available to those skilled in the art.

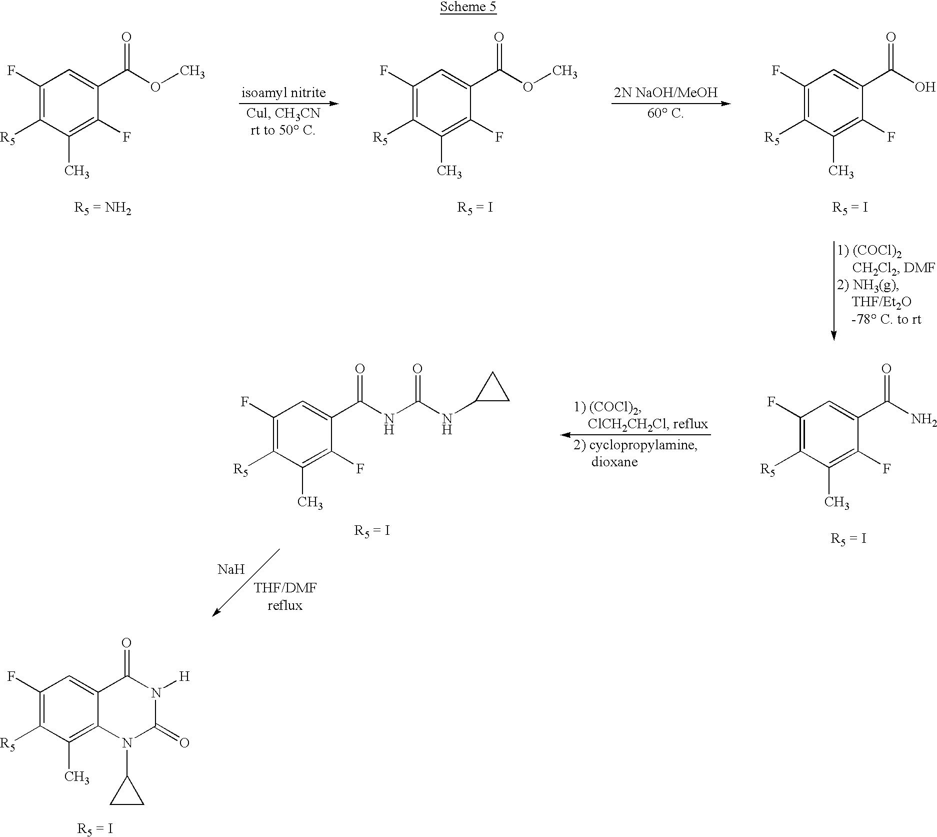

Scheme 6 provides an approach to invention compounds wherein R₆ is a fluorinated alkyl group. Thus, treatment of 2,4,5 trifluorobenzoic acid with lithium hexamethyldisilylazide and dimethyl formamide gives the R₆ aldehyde, which, when treated with (diethylamino) sulfur trifluoride (DAST) gives the R₆ difluoromethyl compound. Ring closure to provide the quinazolinedione scaffold is achieved as provided in earlier schemes.

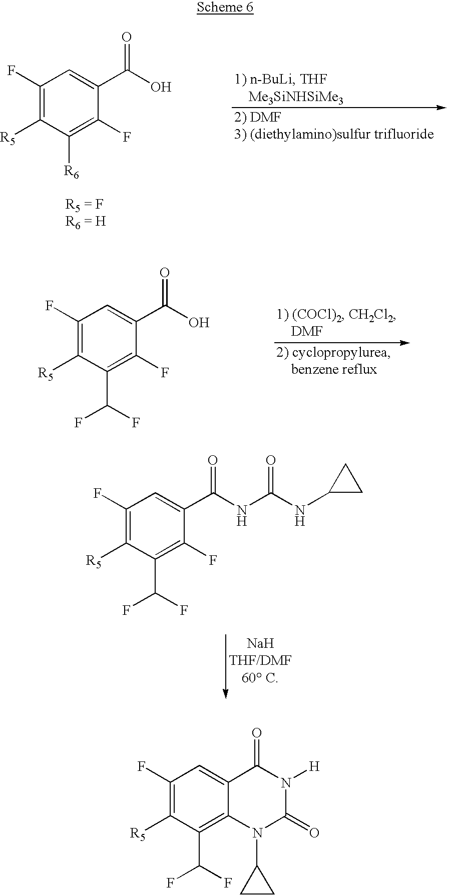

Scheme 7 provides an approach to compounds of the invention wherein R₅ is a substituted cyclopropyl group. The para position in ethyl (2,4,5 trifluoro-3-methyl) benzoate is activated relative to the ortho or meta position. Thus, reaction of the starting compound with the shown cyano ester in the presence of base provides the para addition product. Saponification of the t-butyl ester, followed by decarboxylation under acidic conditions provides the R₅ cyanomethyl compound. Treatment of the R₅ cyanomethyl compound with benzyl triethyl ammonium chloride and 1,2 dibromethane gives the R₅ cyanopropyl compound. Quinazolinedione formation then occurs via cyclization upon treatment with base to provide the target compound.

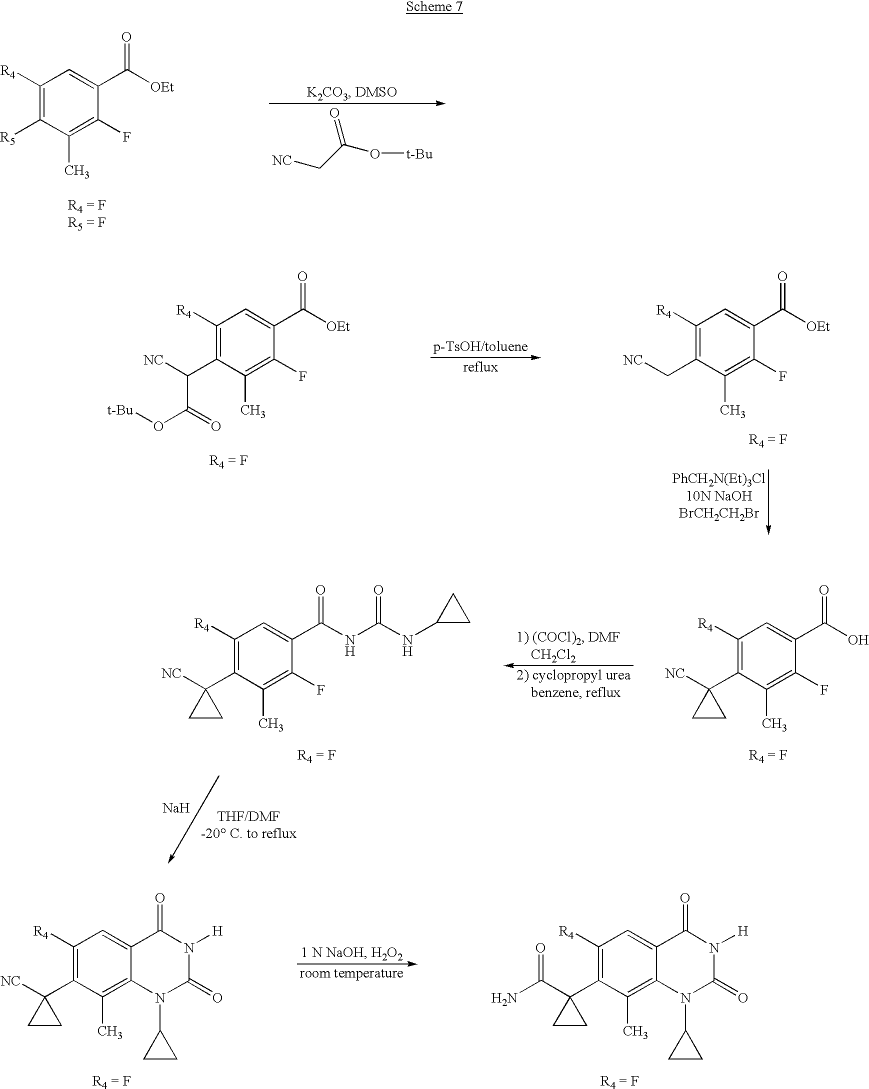

Schemes 8A-C provide approaches to invention compounds wherein R₆ is an alkoxy group and J may be N or C. Scheme 8-A summarizes the general approach. Esterification of the meta-hydroxy benzoic acid derivative, followed by O-alkylation of the meta-hydroxy group using the t-butyl ester of bromoacetic acid provides the shown aryl ether. Hydrolysis of the t-butyl ester, followed by fluorination gives the fluoromethoxy compound. Quinazolinedione ring formation can be effected as provided in earlier schemes.

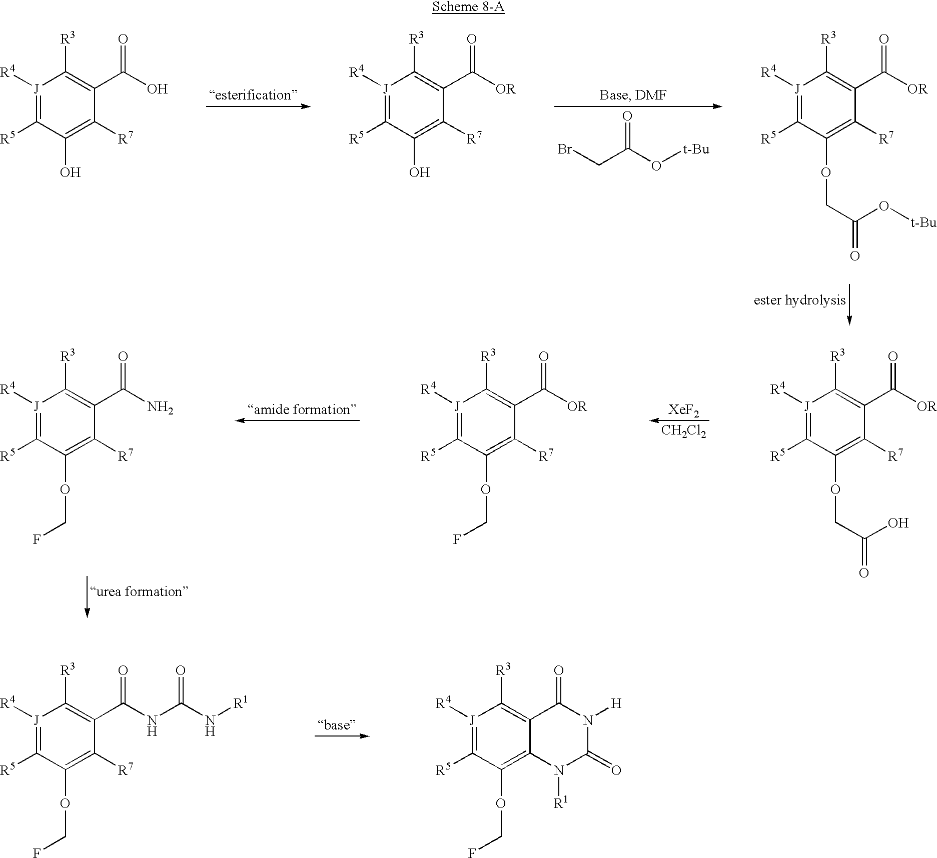

Scheme 8-B provides a particular example of the Scheme 8A approach. In 5 this variant, fluorination is effected using xenon difluoride (XeF₂) in a chlorinated solvent (Shaw, et. al, J. Am. Chem. Soc. 91, 1563 (1969).

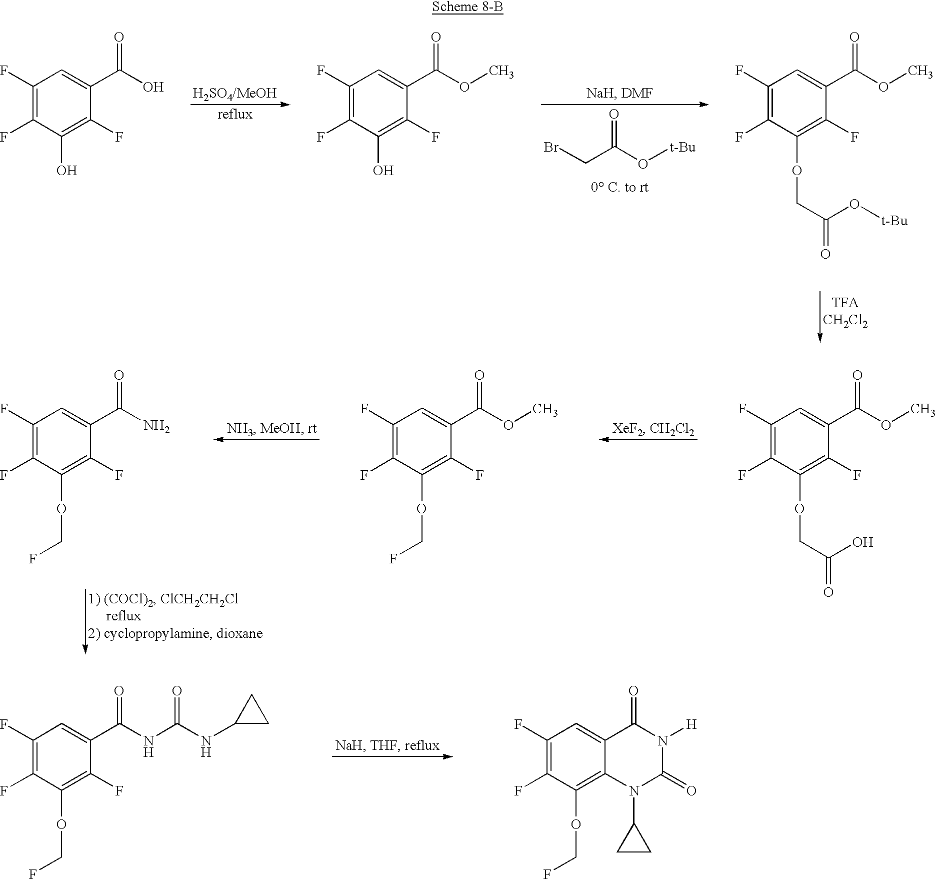

Scheme 8-C provides an approach to the synthesis of an invention compound with an R₆ difluoroalkoxy group. Thus, 2,4 difluoro-3-methoxy benzoic acid is converted to the amide. The methoxy group is then converted to a phenol using boron tribromide. O-alkylation of the phenol with dichlorodifluoromethane in the presence of base, followed by hydrogentaion, provides the difluoromethoxy compound. Formation of the quinazolinedione ring scaffold can then be achieved as provided earlier.

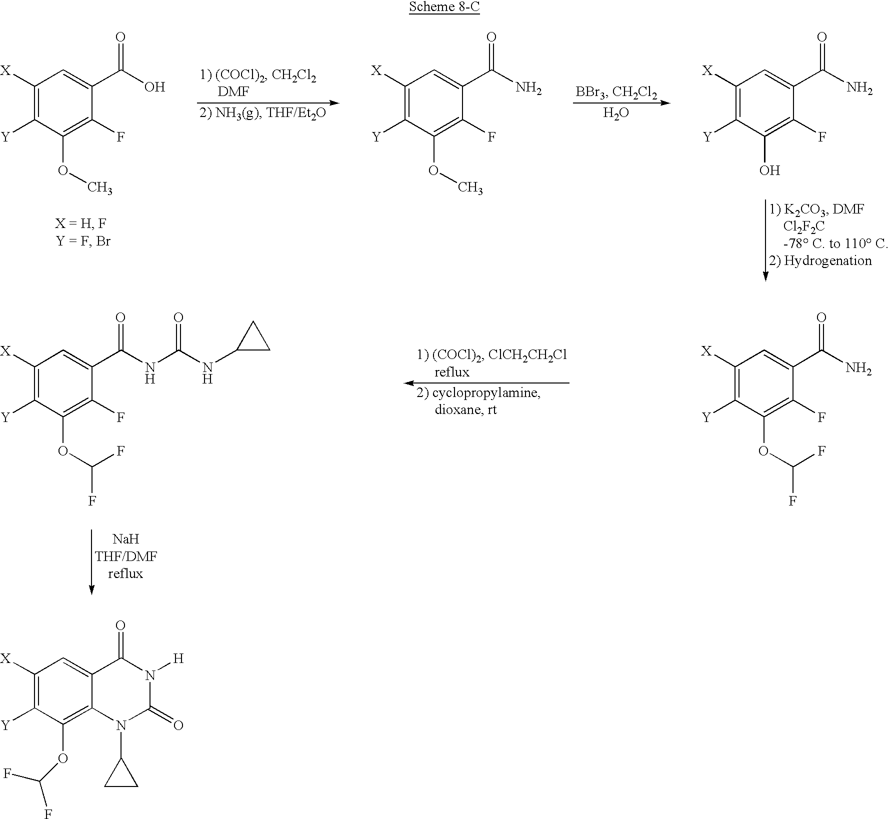

Scheme 9 provides an approach to the synthesis of tricyclic invention compounds. Thus, the indole starting material can be converted to the shown methyl ester using standard procedures. Removal of the thioethyl group using Raney Nickel is followed by reduction of the indole double bond using trifluoroacetic acid/triethyl silane to provide the cyclization precursor. Treatment of the cyclization precursor with trifluoroacetic acid and potassium isocyanate in a chlorinated solvent gives the tricyclic invention compound.

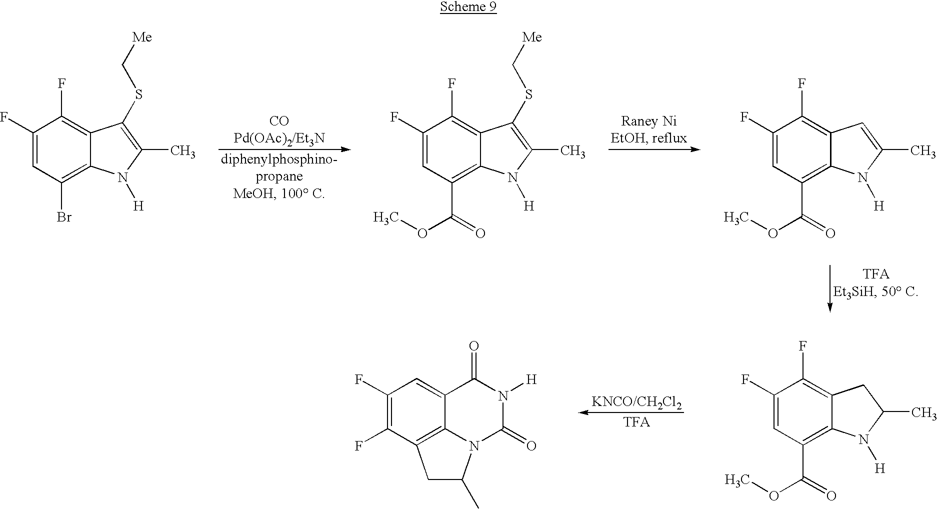

Scheme 10 provides an approach to compounds of the invention wherein R₃ is alkyl or alkoxy as defined herein. Thus, a compound wherein R₃ is H and R₆ is alkyl or alkoxy can be treated with a base, such as lithium diisopropyl amide (although other bases may be used), followed by an alkylating or acylating agent. Alkylating agents (such as alkyl halides, -mesylates, triflates, and the like) and acylating agents (such as acid halides, acid anhydrides, phosphoryl halides, and sulfonyl halides, among others) are well known in the art and many are commercially available, for instance from a supplier such as Aldrich, and are listed in the Aldrich Handbook of Fine Chemicals, 2002-2003. Other alkylating or acylating agents can be prepared as needed according to methods available to the skilled artisan. An R₅ sidechain can then be attached to the resulting compound wherein R₃ is an alkyl, acyl, or other group.

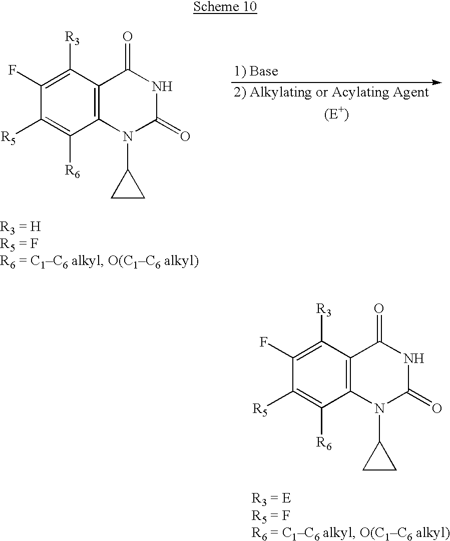

Other approaches to the preparation of the invention compounds are available to the skilled artisan. For example, a general synthetic strategy for core formation suggested by WO 01/53273, which is assigned to the same assignee as the instant application, can be substantially modified and adapted to the synthesis of the invention compounds disclosed herein. Thus, Scheme 11 discloses another approach to the invention compounds wherein the R₁and R₅ sidechains are attached prior to cyclization to form the quinazolinedione ring. As provided by the scheme, a difluoro substituted benzoic acid wherein one or both of J or K may be N is reacted with oxalyl chloride or an equivalent acylating reagent (such as an acid anhydride), and the acid halide or anhydride is reacted with an alcohol (ZOH) to afford the respective ester (Z is C₁-C₆ alkyl such as methyl, ethyl, isopropyl, etc.). The ester is reacted with an amine, for example, a heterocyclic amine, to produce the desired 4-heterocyclic phenyl derivative. Alternatively, carbocycles and aryls may also be introduced at this 4-position using palladium catalyzed couplings of tin or boronate carbocycles and aryls, with starting materials containing a Br, I, or triflate at the 4-position as described by Suzuki A., Pure Appl. Chem., 1994;66(2):213-222 and Stille J. K., Angew. Chem. 1986;98(6):504-519.

Reaction of the 2-fluoro benzoic acid analog with a primary amine R₁NH₂ affords the corresponding anthranilic acid ester. The ester group is readily hydrolyzed by reaction with an acid such as hydrochloric acid or a base such as sodium hydroxide to give the corresponding polysubstituted anthranilic acid. The acid is then coupled to a source of NH₃, typically protected by a protecting group (Pro), to provide the corresponding amide. The amide may then undergo cyclization in the presence of a carbon monoxide equivalent such as phosgene to provide the quinazolinedione. At this point, the protecting group may be removed to give the 3-hydridoquinazolinedione of Formula I, which may be a final product of the invention, or may be further derivatized. The protecting group (Pro) of the N-protected quinazolinedione is removed by conventional methods such as hydrogenation, treatment with acid, lewis acid, or base, or metal catalysis to afford the shown invention compound A.

If R₃ is a leaving group such as F, it may be activated towards displacement with a nucleophile HY-Pro′ where Y is NH or O. Other R₃ groups such as chlorine, bromine, or sulfonyl are also good leaving groups. The displacement generally is carried out in a solvent such as ethanol, DMSO, DMF, THF, and at a temperature of about 0° C. to 120° C.

The protecting groups (Pro and Pro′) may be selectively removed by hydrogenation, acid or base treatment, metal catalysis, or other standard methods. When Pro or Pro′ is methoxy or benzyl, either of these groups may be removed with Pd/C and hydrogen. A t-butyl oxycarbonyl group may be removed by alcoholic HCl, TFA, or TFA in dichloromethane, ethyl acetate or diethyl ether. Allylic oxycarbonyl groups may be removed by PhSiH₃ and Pd catalyst. Solvents such as alcohol, THF, alcohol/THF, alcohol/THF/DMF, diethyl ether, etc. are generally employed in such protecting group cleavage reactions.

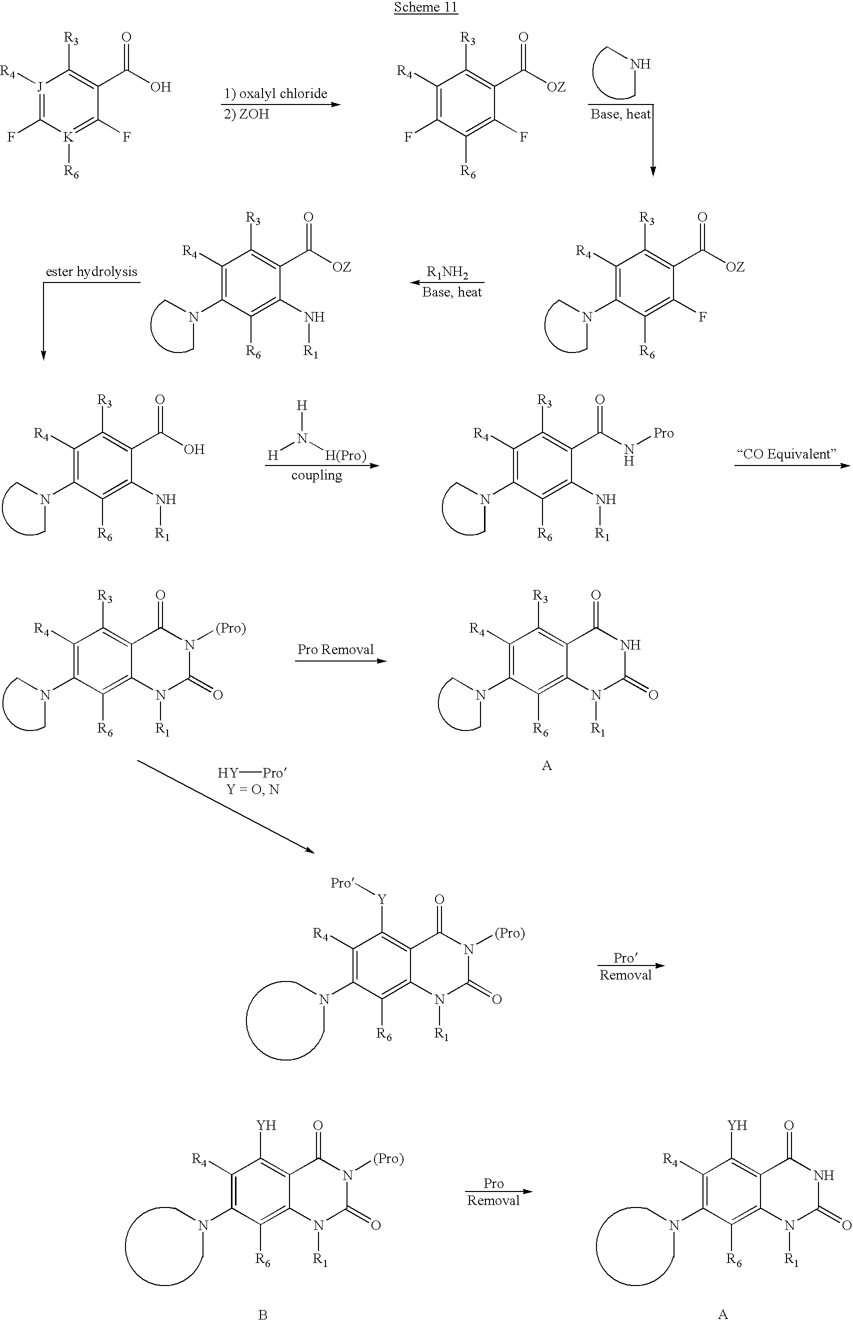

In Scheme 12, an ortho-aminobenzoic acid wherein one or both of J or K may be N is utilized as the starting material, and is alkylated on the amino group. For example, when R₁ is cyclopropyl, the alkylation is carried out according to the method of Gillaspy (Tetrahedon Letters, 1995:7399) to provide the cyclopropyl amine. When R₁ is phenyl or substituted phenyl, the respective amine is prepared from the ortho-fluorobenzoic acid using a base, such as, lithium diusopropylamide or lithium hexamethyl disilazide, and the appropriate aryl amine (R₁NH₂). When R₁ is any alkyl group, such as t-butyl or isopropyl, the R₁ can be introduced by reacting the amine and ortho halo benzoic acid with a Cu catalyst such as copper bronze, or cuprous acetate in the presence of a base such as potassium acetate, triethylamine, or pyridine.

The resulting R₁-substituted amino benzoic acid is then coupled to ammonia in an appropriately protected form to provide the corresponding benzamide using methods described in the literature. The corresponding amide can be further reacted, if R₅ is a leaving group such as fluoro, with various heterocyclic amines (e.g., piperidine or pyrrolidine) to form the desired 4-heterocyclic benzamide derivative. Alternatively, carbocycles and aryls (e.g., cyclobutyl or phenyl) may also be introduced at this 4-position using palladium catalyzed couplings of tin or boronate carbocycles and aryls, if the starting material contains a Br, I, or triflate at the 4-position.

The 4-substituted benzamide derivative is then cyclized to generate the quinazolinedione by reaction with carbonyldiimidazole (CDI), phosgene, triphosgene or the like in ethereal solvents such as diethyl ether, chlorinated hydrocarbons such as dichloromethane, or aromatic hydrocarbons such as toluene, in the presence of a base such as triethylamine or sodium bicarbonate (NaHCO₃).

Alternatively, the corresponding amide is first cyclized and then the R₅ halo group is displaced by reaction with a carbocyclic amine to afford the same product. Deprotection by conventional methods provides the invention compound A.

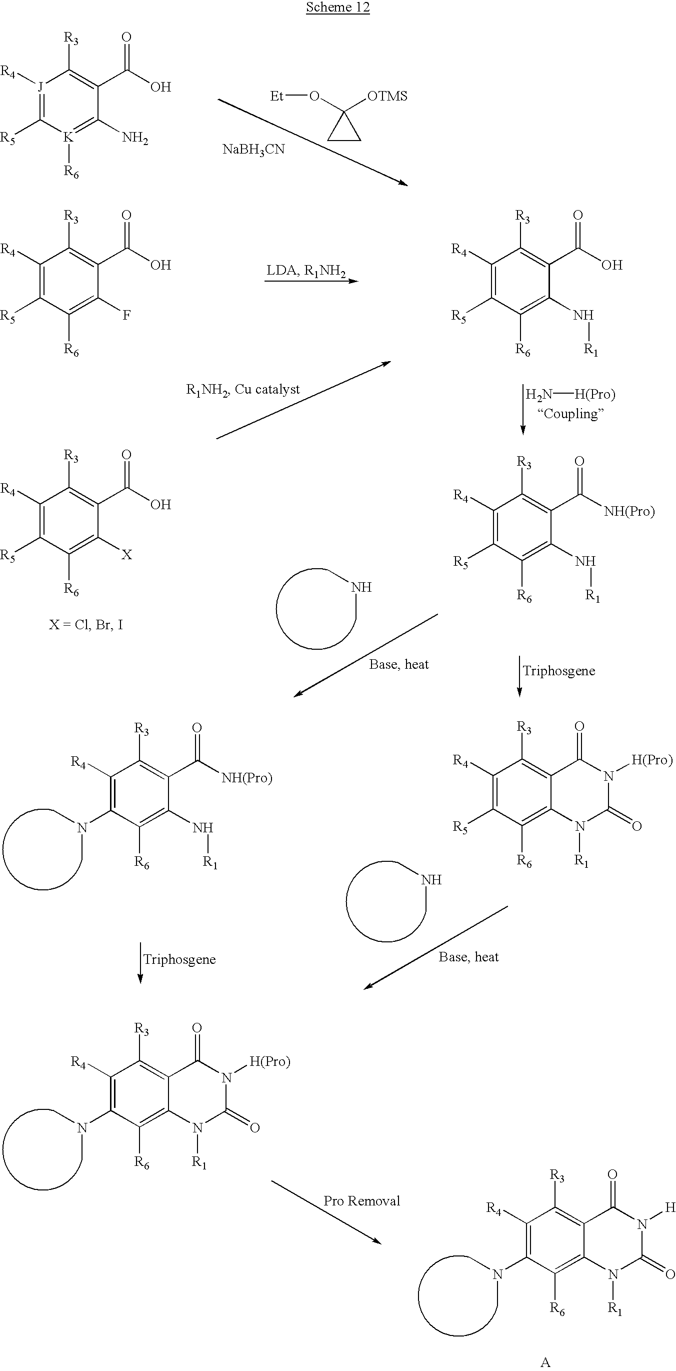

Scheme 13 illustrates alkylation at the 1-position of a quinazolinedione to provide invention compounds wherein R₁ is alkyl. Thus, a 2-aminobenzoic acid wherein one or both of J or K may be N is reacted with an N-protected amine to provide the corresponding N-protected amide. This intermediate can then be reacted with a carbon monoxide equivalent such as phosgene or phosgene/base in an ethereal solvent, or with a phosgene equivalent such as triphosgene in a chlorinated hydrocarbon such as dichloromethane, to give the quinazolinedione. The alkylation of the quinazolinedione to provide a 1-alkylated-quinazolinedione is accomplished by reaction with an alkyl halide as described by Bouzard, supra., 1990. Typically, such reactions are carried out in THF, ether, DMSO, an alkanol, or DMF, and in the presence of a base. Typical alkyl halides (R₁X where X is halo) include ethyl iodide, ethyl bromide, cyclopropyl iodide, n-decyl bromide, and the like. Typical bases include sodium hydride, potassium carbonate, and the like. Conversion of the 1-alkylated-quinazolinedione to other invention compounds (and removal of protecting groups such as benzyl) can be carried out as provided earlier to give the corresponding 1-alkyl (R₁=alkyl) 3-hydrido compounds such as A.

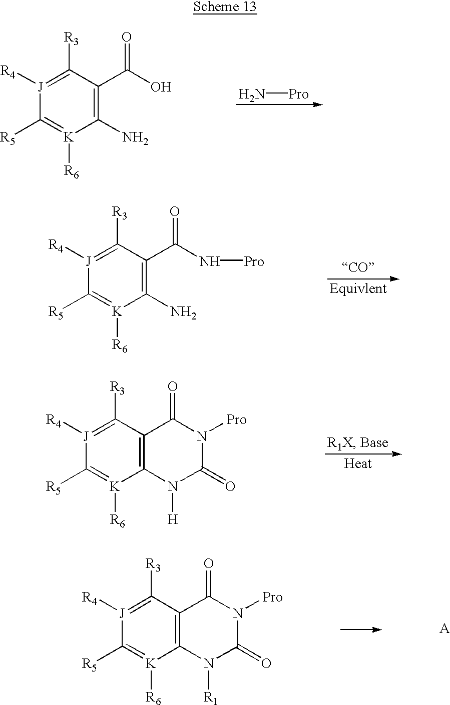

Displacement of leaving groups located at the R₅-position of the quinazolinedione (e.g., R₅=halo) as shown above is not limited to nitrogen heterocycles. Other nucleophiles (Nu) such as CH₃O—, N₃—, R′R″NH, R′—NH₂, and R′S— (where R′ and R″ are each independently (C₁-C₇)alkyl) also displace a leaving group such as F, Cl, or NO₂ at the R₅-position as provided in earlier schemes. When the leaving group is a triflate or higher halide (Br or I), organotin reagents or organoboronates may be used with palladium catalysts to deliver a carbon nucleophile. The methodology generally disclosed in Scheme 14 may be adapted to follow the coupling methodology of Stille et al., Angew. Chem. Int. Ed. Eng., 1986;25:508, further exemplified by Mitchell (Synthesis, 1992:803). It is to be understood that in Scheme 14, one or both of J or K may be N.

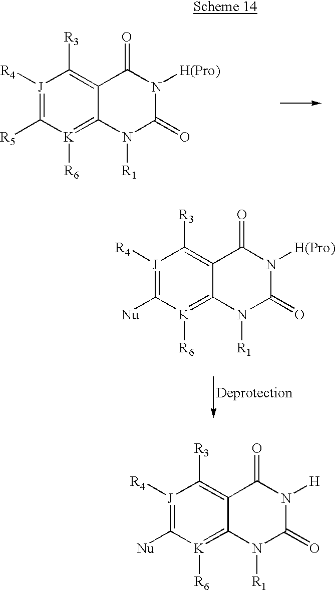

All of the chemistry depicted and described in Schemes 11 to 14 is applicable to make compounds of Formula I wherein J and K both are carbon, or where one or both of J and K are N. When either J or K is nitrogen, the displacement reactions described above may be even more facile than when J and K are both carbon.

As stated above, compounds of Formula I wherein one or both of J or K are nitrogen may be prepared by Schemes 11-14, or alternatively, by routes which take advantage of the activation of leaving groups ortho and para to the J and/or K nitrogen atom. Such routes will systematically introduce R₅ and R₁ groups as desired. This methodology also applies to cases in Formula I where K-R₆ is C—H or C—F and J-R₄ is C—F. Such systematic substitutions are illustrated in Scheme 15.

For example, a pyridine amide has leaving groups such as halo on both sides of the nitrogen. Such groups are generally Cl, but Br, I, F; alkylthio, and sulfoxides, such as methyl sulfoxides, are also good leaving groups for such compounds. These leaving groups may be sequentially displaced based on reactivity. In Scheme 15, where J=N or J-R₄═CF, the 4-chloro (para to the aminocarbonyl group) is displaced preferentially (relative to the 2-chloro group) using a nucleophilic amine such as diethylamine, pyrrolidine, methylpiperazine, and the like to give the corresponding amino substituted analog. This analog can then undergo reaction with R₁NH₂ to displace the second leaving group (e.g., the 2-chloro group). The resulting 2,6-disubstituted-pyridylamide is then reacted with carbonyl diimidazole (CDI), phosgene, or other carbon monoxide equivalents to form the cyclized quinazolinedione product.

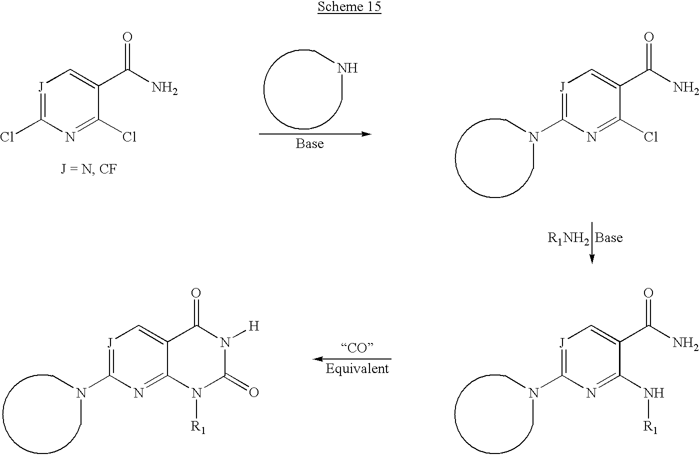

Tricyclic compounds (i.e., where R₁ and R₆ in Formula I are taken together with the atoms to which they are attached to form a ring) can be prepared according to Schemes 16-19. Schemes 16-19 differ in the introduction of the R₁ substitutions in Structures B and C wherein R₅ is as defined above for Formula I. In Scheme 16, the ortho-fluoro nitro compound (other leaving groups such as chlorine, bromine, and sulfonyl may also be employed in place of fluoro) undergoes reaction with the shown ester. The nitro group is then reduced using, for example, Raney N₁, H₂ over Pd/C, or an active metal in acid such as iron or tin in HCl or acetic acid. The newly formed amine readily cyclizes with the ester (other acid analogs may be employed such as thioesters, amides, and the like). The cyclized product is then reduced with hydride reducing agents such as LiAlH₄ and the like to produce the dihydroquinoline derivative, which in turn is reacted with chloral hydrate and then an acid to form a dione ring. The dione ring is subsequently opened using, for example, sodium hydroxide and hydrogen peroxide to give the benzoic acid. The quinazolinedione ring is then prepared using the chemistry described earlier to give a precursor B to the invention compound.

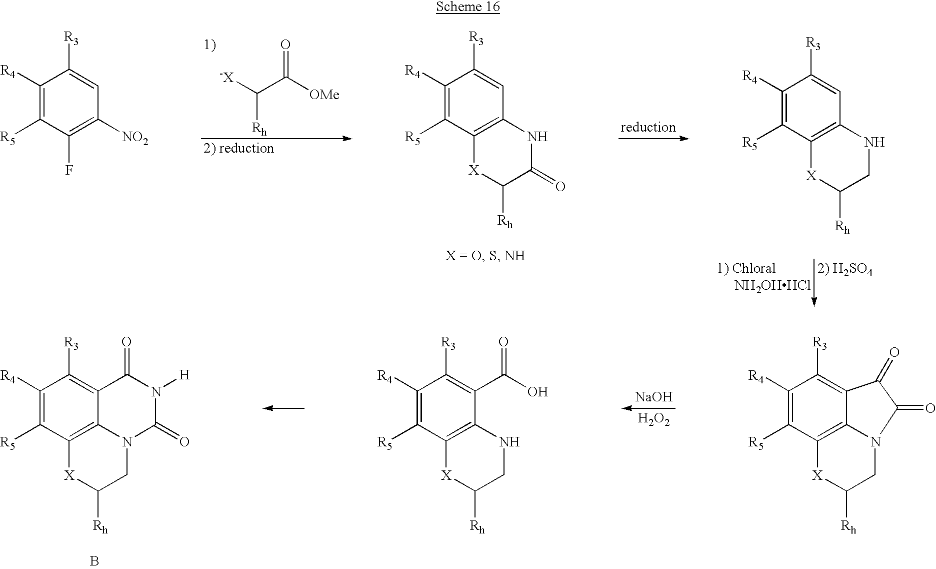

In a similar series of reactions depicted in Scheme 17, the ortho-fluoro nitro compound is reacted with an α-nucleophile substituted ketone, and the resulting product is likewise reduced. In this sequence, the resulting aniline forms a cyclic imine, which is further reduced with H₂ over Pd/C or by chemical hydride reducing agents such as sodium borohydride or sodium cyanoborohydride to give the dihydroquinoline. Such reductive aminations are well-known in the art and are typically performed in THF, alcohol, water alcohol mixtures, or in water DMF mixtures. The remaining steps to produce C follow those of Scheme 16. When the R₅ substituent is a leaving group (e.g., R₅=halo), compounds B and C may be further reacted with nucleophiles (such as pyrrolidine or piperidine) to give compounds of Formula I as in the previous schemes. Also, as indicated in Schemes 17-19, R_{h′} can have any of the meanings disclosed for Rₕ.

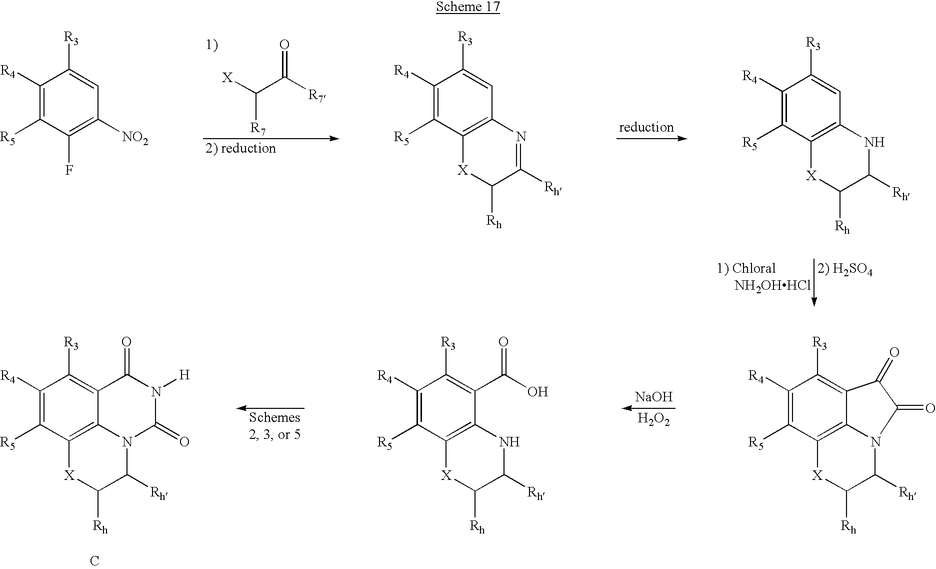

In Scheme 18, an R₃ amino group is attached to the tricyclic compound via nitration and reduction.

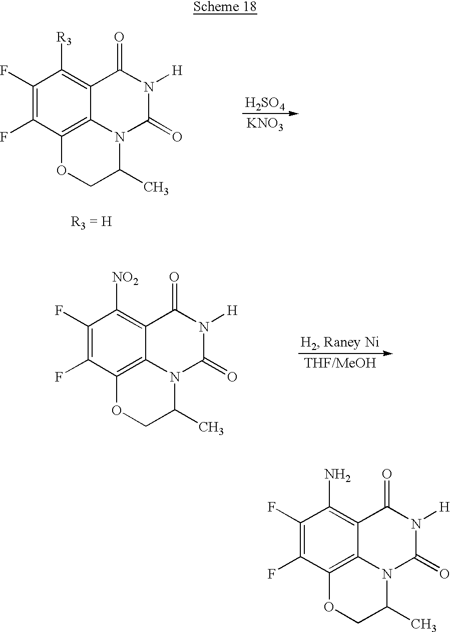

As depicted in Scheme 19, target tricylic compounds such as C are prepared in a slightly different manner. In this variant approach, XH (wherein X is O, S, or NH, for example) is attached to the phenyl ring of the starting aniline, and a leaving group L (such as halo) is attached alpha to the ketone reactant. The nucleophile may be activated with bases such as sodium hydride or potassium hydride, triethylamine or 1,8-diazabicyclo[5.4.0]undec-7-ene (DBU), or sodium, potassium, or cesium carbonate to displace the leaving group L. In this sequence, the resulting aniline forms a cyclic imine, which is further reduced with H₂ on Pd/C or by chemical hydride reducing agents such as sodium borohydride or sodium cyanoborohydride to give a dihydroquinoline. Such reductive aminations are well-known in the art and are typically performed in THF, alcohol, water alcohol mixtures, or in water/DMF mixtures. The dihydroquinoline intermediate is reacted with chloral hydrate, and then an acid to form the dione ring. The dione ring is subsequently opened by reaction with a base, for example sodium hydroxide and hydrogen peroxide, to give the benzoic acid.

The quinazolinedione ring is then prepared by first forming an ester on the benzoic acid as provided earlier, followed by reaction of the benzoic acid ester with chlorosulfonylisocyanate or the like at temperatures of 0° C. and below, followed by treatment with a base such as triethylamine or diisopropylethylamine to provide compounds of structure C. When R₅ is a leaving group such as Cl or F, the invention compound precursor B (in Scheme 16) and the invention compound D (in Scheme 19) can be prepared by coupling to the R₅ a side chain, e.g., various heterocyclic amines CNH to produce the desired derivatives. Alternatively, carbocycles and aryls may also be introduced as R₅ side chains using palladium catalyzed couplings with tin or bornate carbocycles and aryls, for example when R₅ is a Br, I, or triflate.

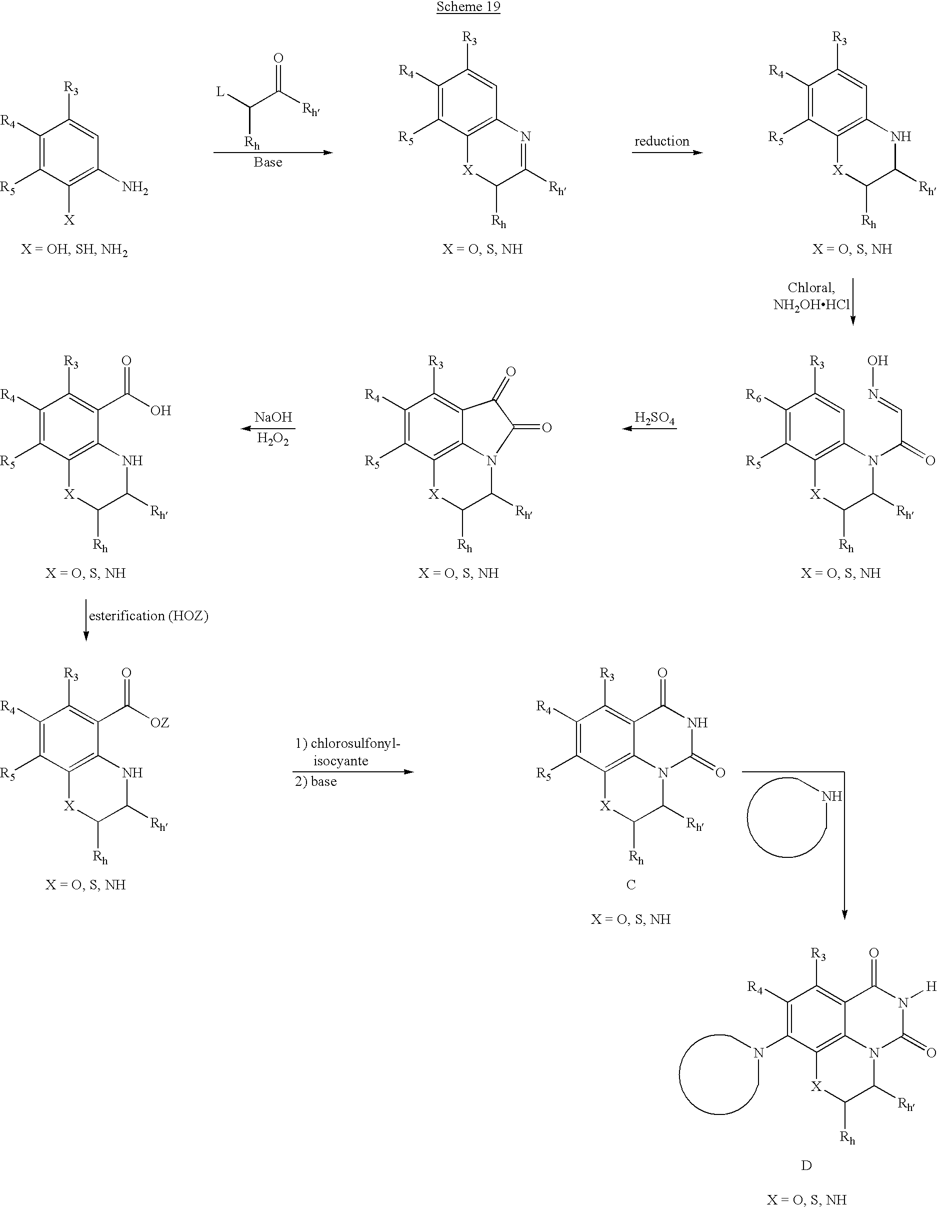

It should be noted from Schemes 16-19 that Rₕ and R_{h′} will form chiral centers, giving R and S enantiomers and diastereomers. Such enantiomers or diastereomers may be separated, if desired, by chiral HPLC at any stage. Resolution of any of the intermediates may be performed with techniques of fractional crystallization using mandelic acid, tartaric acid, or other chiral, optically pure acid resolving agents. Chiral benzylic amines (such as α-methylbenzyl amine) can be used in the preparation of starting materials for the above schemes, and chiral amides may also be prepared using chiral acids, such as mandelic acid and the like. The isomers can then be separated and the chiral amine can be hydrogenated, or the chiral amide can be hydrolyzed.

Scheme 20 illustrates synthesis of compounds of Formula I wherein one or both of R₄ and R₆ are halo via halogenation. The halogenation is carried out on a 2-aminobenzoic acid where one or both of R₄ and R₆ is hydrogen. If both R₄ and R₆ in the benzoic acid are H, then halogenation can be accomplished at both positions selectively or simultaneously. Thus, for example, chlorination at R₄ or R₆ is achieved by reaction of the benzoic acid with N-chlorosuccinimide, t-butylhypochlorite, chlorine gas, and the like. Similarly, bromination at R₄ and R₆ can also be accomplished by reaction of the benzoic acid with Br₂, N-bromosuccinimide, and the like. Such halogenations are well-known in the art. Halogenation thus provides the respective mono- or dihalo-compound. The halogenated benzoic acid then can be further reacted as shown in earlier schemes to provide the quinazolinediones of Formula I.

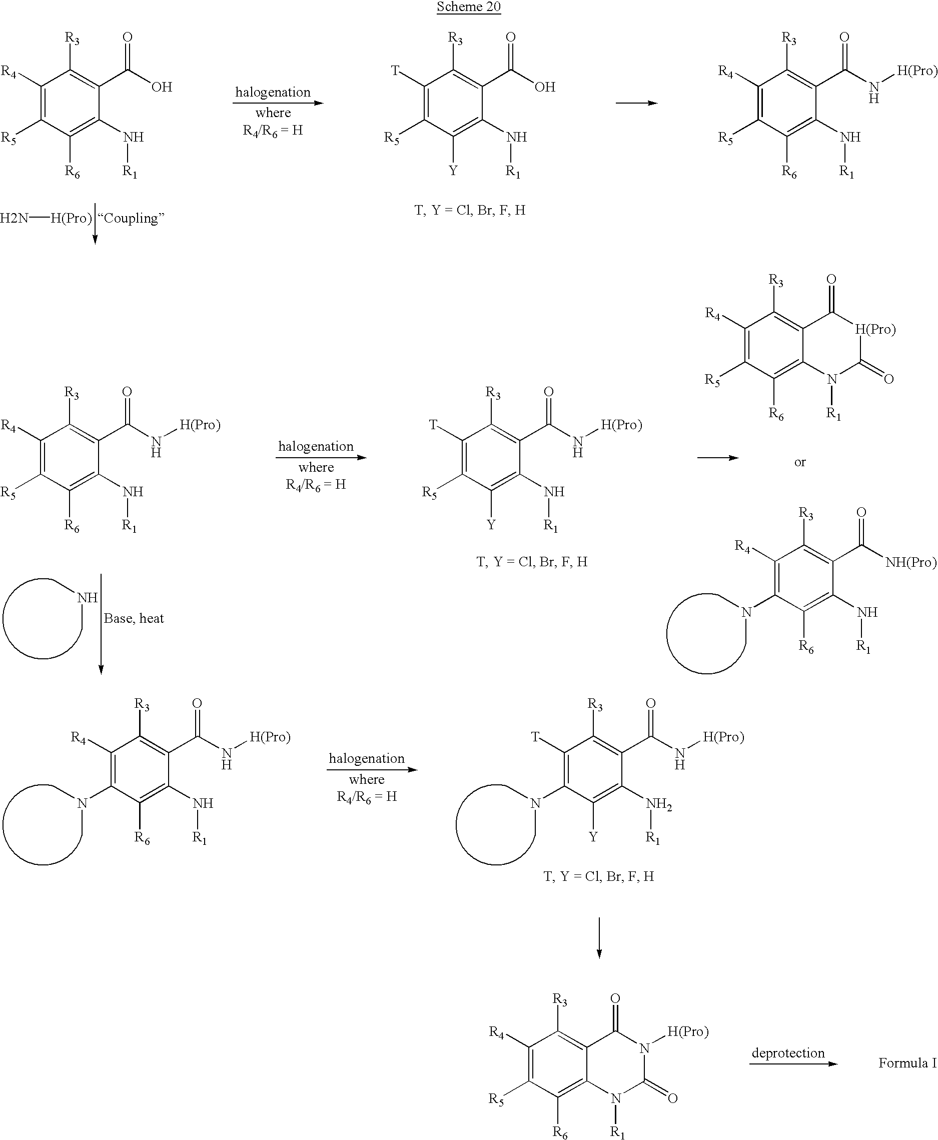

In Scheme 21, compounds where R₆ is H are halogenated as described above to provide the corresponding 3-halo-2-aminobenzoic acid (Y=halo). This intermediate can then be diazotized by reaction of the 2-amino group with sodium nitrite or t-butyl nitrite, which is then converted to a 2-halobenzoic acid in the presence of an appropriate sodium, potassium, or copper salt such as sodium iodide, potassium chloride, or the like. The resulting 2,3-dihalobenzoic acid (where X and Y both are halo) is then converted to the 3-halo-2-aminobenzoic acid by reaction with an amine R₁NH₂ in the presence of a copper catalyst. The 3-halo-2-aminobenzoic acid is converted to an amide and cyclized to the corresponding 8-halo-quinazolindione.

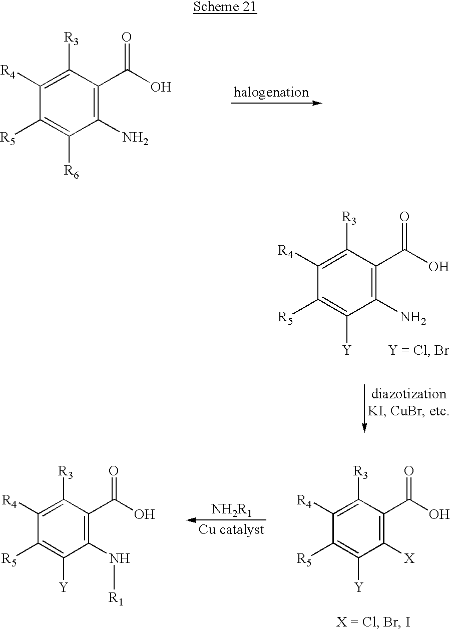

Compounds of Formula I wherein R₄ and/or R₆ is halo such as chloro or bromo are readily dehalogenated by reaction with metal catalysts under hydrogen pressure (Scheme 22). Suitable catalysts include the many variations of Pd on carbon, Raney nickel, or other reagents that are well-known to effect such dehalogenation.

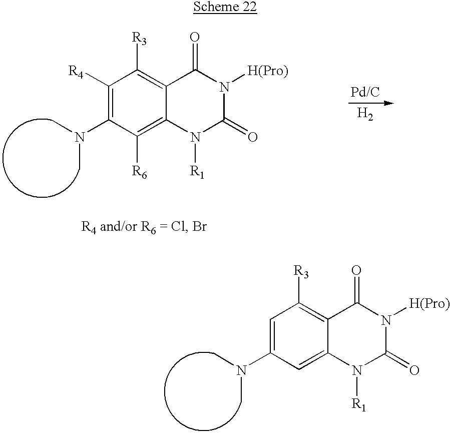

Compounds of Formula I wherein R₄ and/or R₆ are hydrogen can be halogenated to give the mono- or dihalo compound (e.g., R₄ or R₆=Cl or Br). The invention compounds are preferably prepared by first halogenating a benzoic acid derivative, and then cyclizing the halogenated benzoate (Scheme 23). If both R₄ and R₆ are H, then halogenation can be accomplished at both positions selectively or simultaneously. Halogenations can be carried out as described above for Scheme 20. The resulting compound is then converted to the 2-substituted-aminobenzoic acid as depicted in Scheme 11, which is subsequently cyclized.

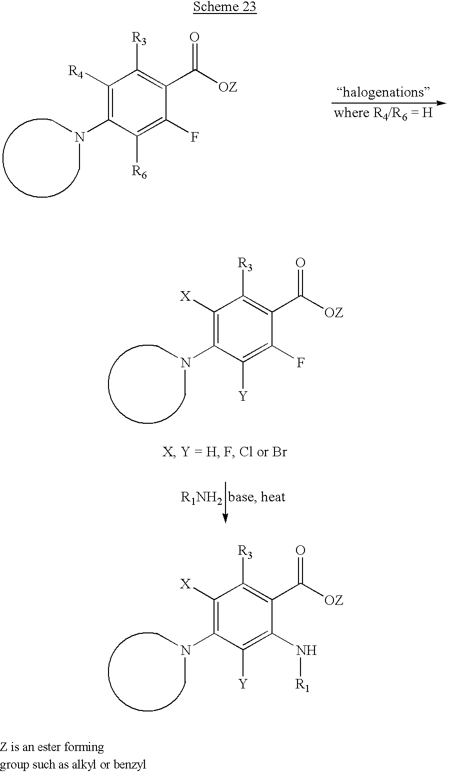

Invention compounds of Formula I can also be prepared as shown in Scheme 24. Substituted benzoic acids can be converted into esters (where Z is an ester forming group such as alkyl or benzyl) by a number of methods known by those skilled in the art. The ester is reacted with an isocyanate such as trimethylsilylisocyanate, chlorosulfanyl isocyanate, and chlorocarbonyl isocyanate, followed by treatment with a base such as triethylamine, sodium t-butoxide or the like, to provide a quinazolinedione. This compound may be further reacted, when R₅ is a leaving group such as fluoro, with various heterocyclic amines. Again, carbocycles and aryls may also be introduced at R₅ if R₅ is a Br, I, or triflate, using palladium catalyzed couplings of tin or boronate carbocycles and aryls. Removal of any protecting groups (Pro) by normal means provides invention compounds such as A.

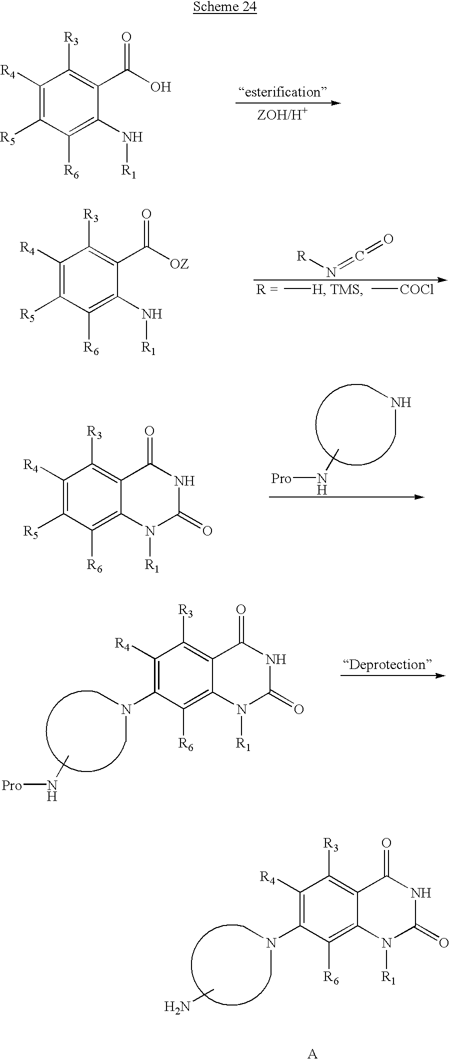

Invention compounds of Formula I having a cyano substituent can be prepared as shown in Scheme 25. For example, a benzoic acid wherein R₆ is hydrogen can be metallated with a strong base such as lithium hexamethyldisilazide or lithium diisopropylamine. The resulting metallated intermediate can then be quenched with dimethylformamide or an equivalent to provide an aldehyde. The aldehyde can be converted to an oxime by reaction with an alkoxy amine. Other electrophiles such as alkyl halides, activated amides, esters and halide sources such as 1,2-dichloro-tetrafluoroethane may also be employed to provide other R₆-substituted benzoic acid derivatives that can be used to make the invention compounds. The oxime is then converted into a cyano group under the cyclization conditions required to form the quinazolinedione ring system (e.g., reaction with phosgene or triphosgene or the like). Deprotection provides invention compounds where R₆ is —CN.

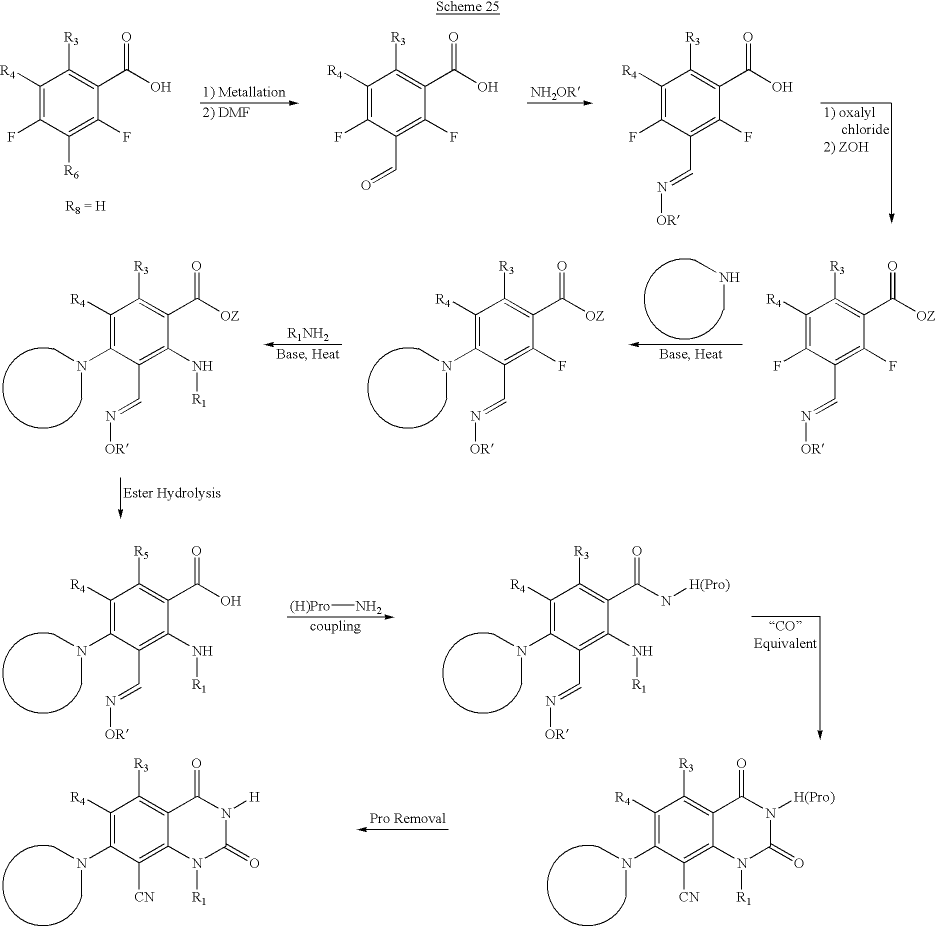

Another alternative for preparing invention compounds is illustrated in Scheme 26. Appropriately substituted benzoic acids can be converted into benzamides by any number of methods as described above. A benzamide can then be treated with oxalyl chloride in a chlorinated solvent such as dichloroethane or an equivalent to provide an isocyanate. The isocyanate is reacted with a substituted primary amine to give a benzoyl-substituted urea. This intermediate can be cyclized to form a quinazolinedione ring system by reaction with sodium hydride, potassium hexamethyldisilazane or other non-nucleophilic bases, generally in a solvent such as tetrahydrofuran (THF)/dimethylformamide, THF with 18-crown-6, THF/dioxane, THF/glyme, THF/diglyme, dimethoxyethane/toluene or an equivalent. The resulting quinazolinedione ring system can then be readily coupled with an appropriately substituted heterocyclic amine (such as those noted above in Table 1) by reaction in the presence of a base such as triethyl amine, diisopropylethyl amine, tetramethyl guanidine, and the like in solvents such as dimethyl sulfoxide, dimethylformamide, dimethylacetamide, sulfolane or the equivalent. Any protecting group associated with the heterocyclic amine side chain is then removed by methods known to those skilled in the art to provide invention compounds having Structure A.

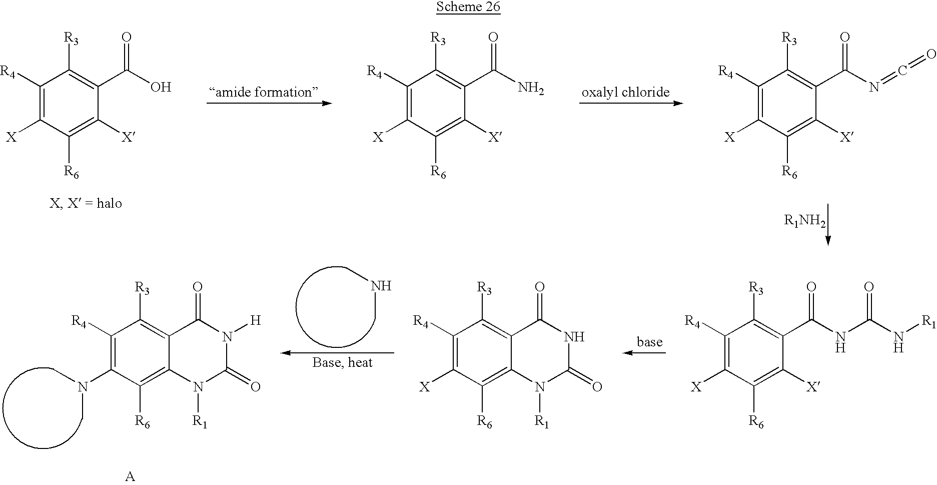

Tricyclic compounds (i.e., where R₁ and R₆, together with the atoms to which they are attached, form a carbocyclic ring in invention compounds of Formula I) can be prepared according to Scheme 27 where a palladium mediated carbon monoxide insertion on a quinoline (X=Br, I, or triflate) under well-precedented conditions gives rise to an ester. The quinoline ring can then be hydrogenated to provide a tetrahydroquinoline by standard hydrogenation conditions. The remainder of Scheme 27 follows the approach of earlier schemes to provide invention compounds such as G, substituted with a displaceable substituent at R₅ (e.g., halo). These compounds may be further reacted with nucleophiles such as amines from Table I to give compounds of Formula I.

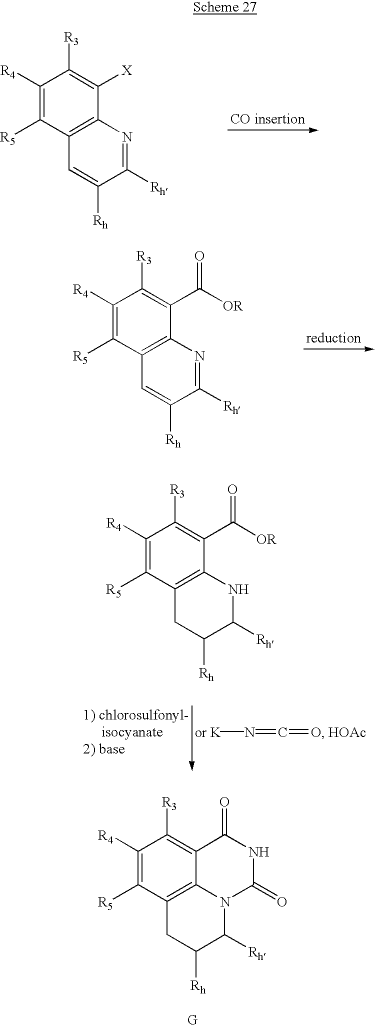

Compounds of Formula I where K is N may be prepared by the routes shown as illustrated earlier or as indicated in Scheme 28, which follows closely the chemistry shown in Schemes 3 and 24. The leaving groups (e.g. halo) ortho and para to the carboxyl group of the pyridyl starting material are highly activated. The two leaving groups ortho to the pyridine nitrogen are generally chlorine, but fluorine, alkylthiol, and sulfoxides such as methylsulfoxide are also good leaving groups for such compounds. The urea intermediate readily cyclizes to the bicyclic system. The R₅ halo leaving group is readily displaced by reaction with an amine

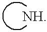

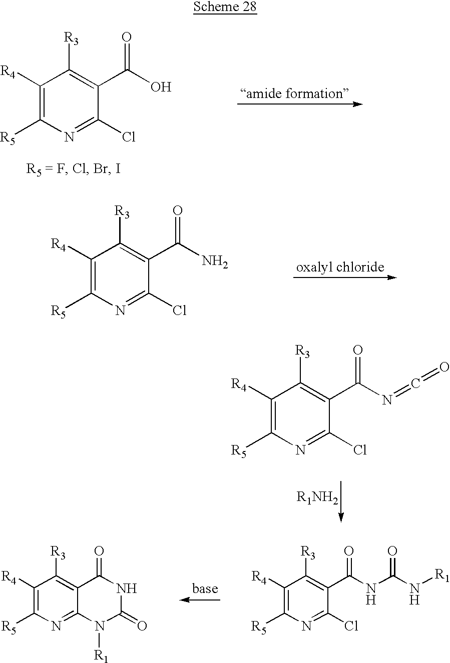

Scheme 29 illustrates the synthesis of benzoic acid starting materials wherein R₅ is bromo. The carboxylic acid moiety of the difluoro substituted benzoic acid first is converted to an ester moiety, by conversion to an acid halide or mixed anhydride using an acid anhydride, mixed anhydride, or acid halide such as oxalyl chloride or the like, followed by treatment with an alkanol. The resulting ester is then reacted with 4-methoxybenzylamine or an equivalent to produce the desired 4-substituted benzoic acid derivative. This intermediate is reacted with triethylsilane and trifluoroacetic acid in a chlorinated solvent such as dichloromethane or an equivalent to provide an aniline derivative. Alternatively, one skilled in the art might also employ transition metal catalysis. The resulting aniline is then subjected to diazotization and converted to a bromide by treatment with cuprous bromide. The ester is then hydrolyzed by well-known methods to the desired benzoic acid, which can be used as a starting material to make compounds of the invention.

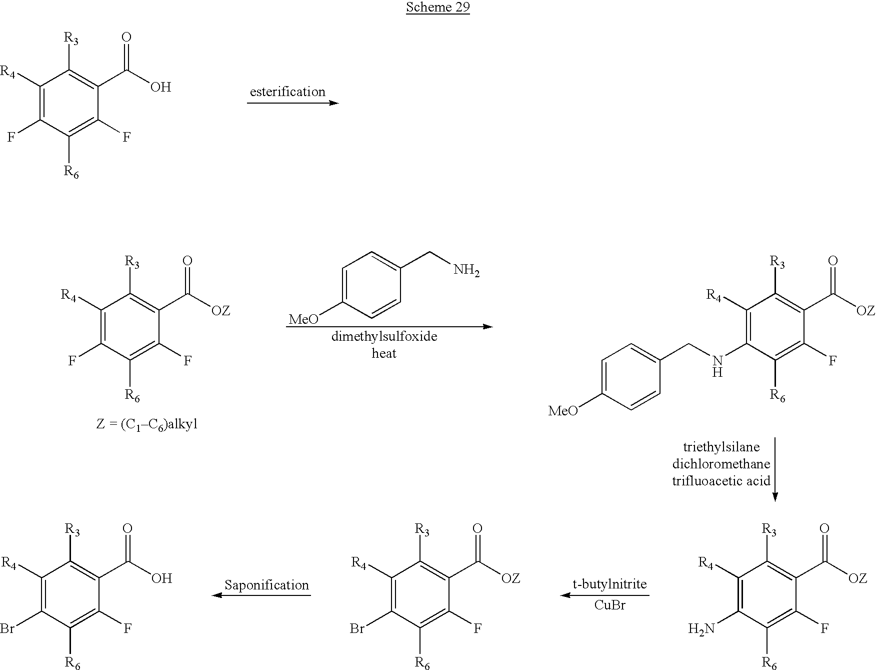

In Scheme 30, 4-bromo-2-fluorobenzoic acid derivatives can be selectively chlorinated by treatment with chlorine gas in chlorosulfonic acid at a temperature of 40° C.-100° C.

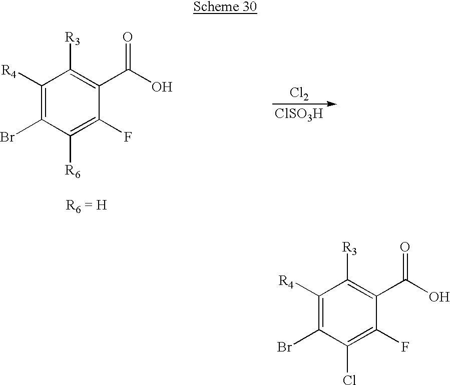

Scheme 31 illustrates the use of 4-bromobenzoic acids, in particular, as starting materials to make invention compounds wherein R₅ is aryl. The 4-bromobenzoic acids are converted into benzamides by any number of methods known in the art. A benzamide is reacted with oxalyl chloride in a chlorinated solvent such as dichloroethane or an equivalent to provide an isocyanate. The isocyanate is reacted with a substituted primary amine to give a benzoyl substituted urea. This intermediate can be cyclized to form a quinazolinedione ring system by reaction with sodium hydride, potassium hexamethyldisilazide or other non-nucleophilic bases in tetrahydrofuran/dimethylformamide, tetrahydrofuran with 18-crown-6, toluene/dioxane, tetrahydrofuran/glyme, tetrahydrofuran/diglyme, glyme/toluene or an equivalent. The resulting 3-aminoquinazolinedione ring system can then be readily coupled with a stannane or boronic acid derivative of a substituted aryl such as phenyl or substituted aromatic heterocycle (Ar).

Alternatively, the 3-position can be protected with a protecting group such as a tert-butyl carbamate, trifluoroacetamide, or 2,5-dimethoxybenzyl group. Protecting groups of these types are well known in the art. The 3-protected quinazolinedione ring system can then be coupled via palladium catalysis with an aromatic (Ar) stannane or boronic acid.

Each of the outlined routes, upon deprotection by standard procedures, provides invention compounds of Formula I where R₅ is aryl such as phenyl or substituted phenyl, or heteroaryl such as pyridyl or substituted pyridyl.

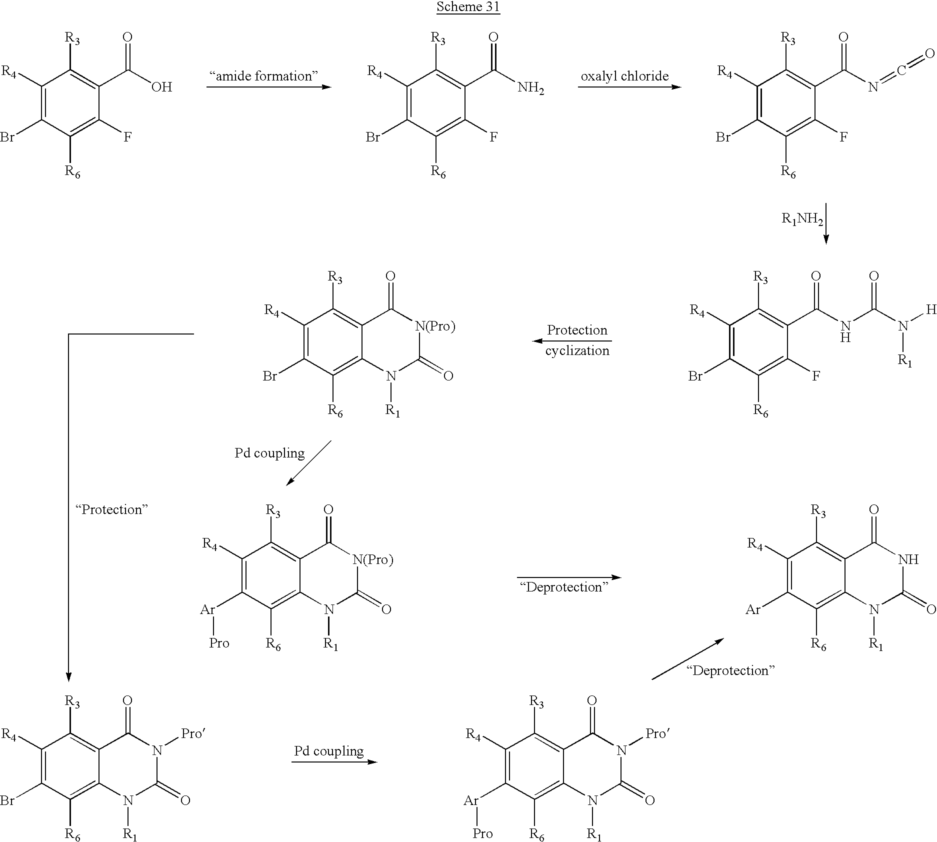

Compounds of Formula I wherein R₅ is a heteroaryl group are alternatively prepared as illustrated in Scheme 32, where R₅ is a substituted thiazole. A 3-protected 7-bromoquinazolinedione is reacted with 1-tributyl-ethoxyvinyltin in the presence of a palladium catalyst. The resulting R₅-substituted adduct is reacted with a brominating reagent such as n-bromosuccinimide and the like in tetrahydrofuran/H₂O to provide an α-bromoketone moety as R₅. Reaction of this intermediate with a thioamide (or thiourea) in a polar solvent such as dimethyl formamide, dimethylacetamide, or ethanol, and at an elevated temperature of about 80° C. to 120° C., effects cyclization to form a thiazolyl group. Deprotection by known methods can then be applied to provide invention compounds of Formula I wherein R₅ is the heteroaryl, optionally substituted with T, which is H, alkyl, substituted alkyl, NH₂, NH-alkyl and N-dialkyl.

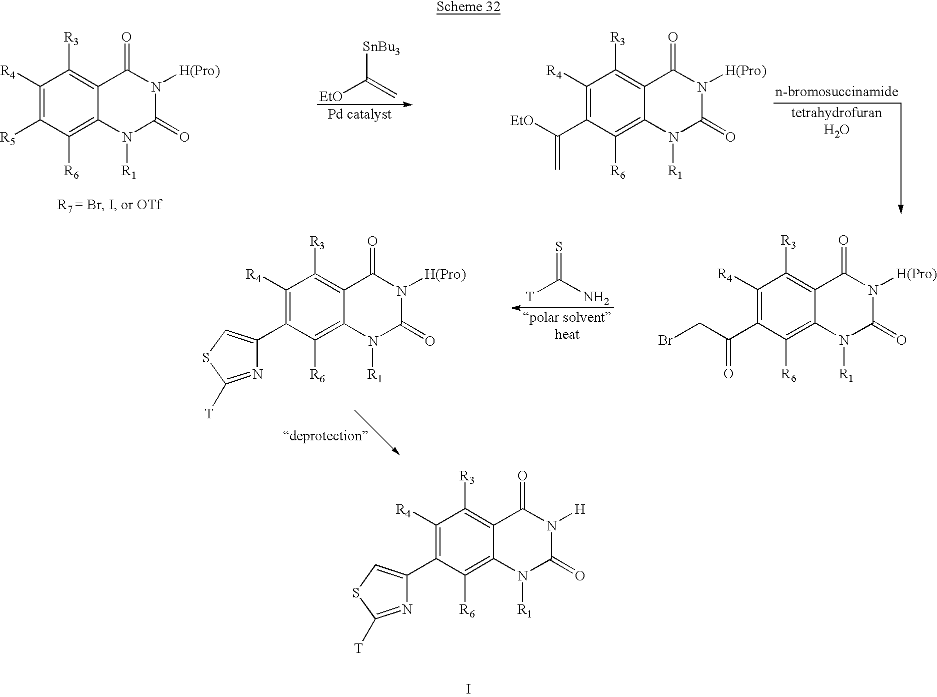

T=H, alkyl, substituted alkyl, NH2, NH-alkyl and N?dialkyl.

Alternatively, R₅-aromatic or heterocyclic aromatic compounds of Formula I are prepared as shown in Scheme 33. A 4-bromo-2-fluorobenzoic acid first undergoes esterification (Z is alkyl or benzyl), and then the ester is reacted with a substituted primary amine (R₁NH₂) in dimethylsulfoxide (DMSO) at elevated temperature of about 100° C. to provide an anthranilic ester. The amine moiety of the antranilic ester is then reacted with an appropriately protected source of NH₂ to provide the corresponding amide. The resulting amido aniline is then cyclized by reaction with phosgene, CDI, or the like, to generate the quinazolinedione. The cyclization typically is carried out in a solvent, such as ethereal solvents, chlorinated hydrocarbons such as chloroform, or aromatic hydrocarbons such as toluene, and in the presence of a base such as triethylamine or NaHCO₃. The quinazolinedione ring system is then further modified as described in earlier schemes to provide the desired R₅-aryl (Ar) compound of Formula I.

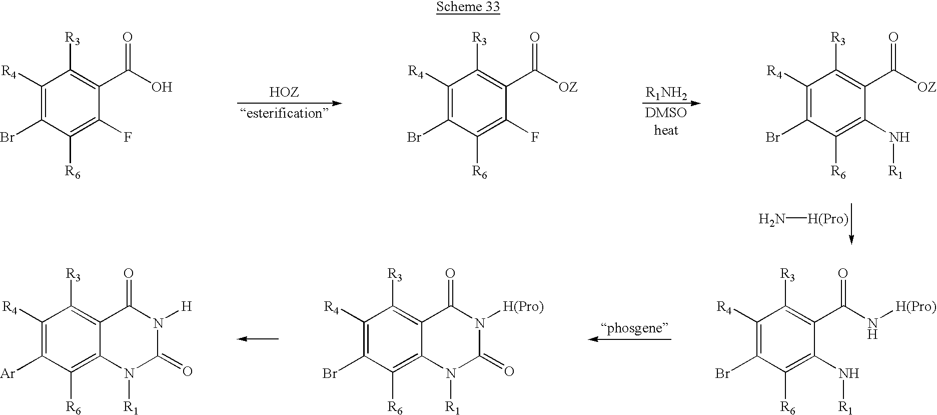

As noted above, R₅ in Formula I is referred to as the “side chain” of the quinazolinedione nucleus. The R₅ side chains are any of those typically found on the quinolone antibiotics. Most of the R₅ side chain reactants required to make the Formula I compounds are readily available from commercial sources. Typical R₅ side chains are shown in Table 1 above.

The synthesis of the R₅ side chains can be accomplished by standard synthetic methods, for example as shown in the following schemes or as found in the literature. Those of ordinary skill in the art will be able to make any of the starting materials required to prepare the invention compounds, although most are available from commercial sources. Some of the sidechains were prepared as disclosed in WO 01/53273 or as otherwise provided in the following schemes.

Scheme A1 generally illustrates the synthesis of typical pyrrolidines, which are preferred side chains (R₅) for invention compounds of Formula I. In Scheme A1, appropriately activated enones can undergo [3+2]cycloadditions under the conditions described by Tsuge et al. (Recent advances in azomethine ylid chemistry. In: Advances in Heterocyclic Chemistry [Katritsky A., ed.] San Diego: Academic Press, 231-349). These reactions are carried out in a chlorinated hydrocarbon solvent such as dichloromethane, chloroform, or dichloroethane and the like, and in the presence of a catalytic acid such as trifluoacetic acid, to provide substituted pyrrolidines (wherein S₁, S₂, S₃, and S₄ independently are alkyl, substituted alkyl, aryl, amino, alkyl and dialkylamino). These intermediates can then be treated with hydroxylamine or any O-alkylated or arylated hydroxylamine under a variety of conditions known to those skilled in the art to provide oximated substituted pyrrolidines. The oximes are reduced to amines with lithium aluminum hydride, diisobutylaluminum hydride, borane, or by selective catalytic hydrogenation. The resulting primary amines can then be protected using a number of methods as described in “Protecting Groups in Organic Synthesis” by Theodora Green (supra). The benzylic pyrrolidine can then be deprotected by hydrogenation, and the resulting pyrrolidine used in the preparation of 7-cyclic amino substituted 3-aminoquinazolinediones of Formula I as shown in the schemes above.

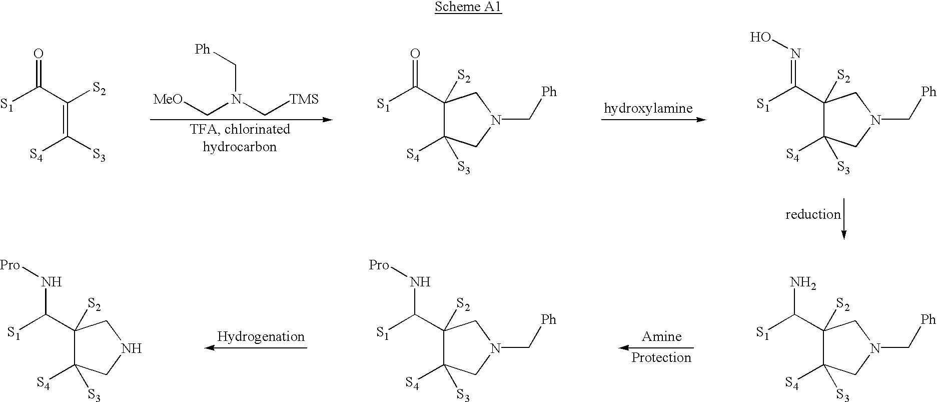

In Scheme A2, enoates (or vinylogous nitriles) may also be used in [3+2]cycloadditions to provide 3- and 4-substituted pyrrolidines. The ester (or nitrile) functionality can then be treated directly with alkyl lithium, -aluminum, or reagents at low temperature (0° C.) to provide S₁ substituted carbonyl groups, or the ester (Z is alkyl or benzyl) can be hydrolyzed under conditions usually employed by those skilled in the art to provide carboxylic acids. This intermediate can then be transformed into an acid chloride with oxalyl chloride and catalytic dimethylformamide in solvents such as dichloromethane or chloroform or into an activated amide (Singh J., Satyamurthi N., Aidhen I., Singh J., Prakt. Chem. [Weinheim, Ger.], 2000;342(4):340-347). An acid chloride can be converted into a ketone using organocopper reagents (Lipshutz B. H., Sengupta S., Org. React. [N.Y.], 1992;41:135-631), and Weinreb amides can be converted into ketones according to the methods described by Weinreb (Tetrahedron Lett., 1981;22(39):3815-3818). The resulting ketones can then be converted into pyrrolidines (optionally substituted with S2, S3, and S₄) as described in Scheme A1. As noted above, pyrrolidines and substituted pyrrolidines are preferred R₅ groups in Formula I.

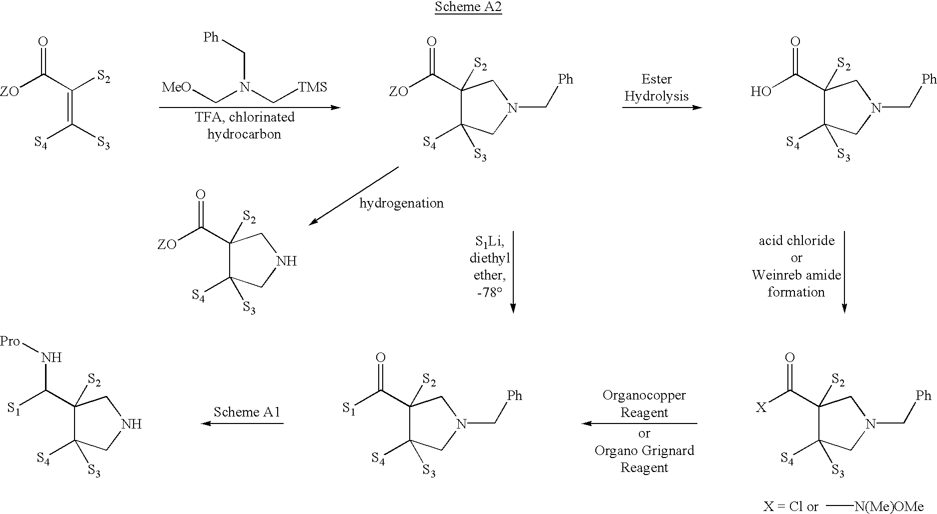

In Scheme A3, 3-carboxylic acid N-benzyl substituted pyrrolidines (Scheme A2) can be converted into amides by a number of methods well-known to those who practice the art of organic synthesis. The amides can then be treated with lithium aluminum hydride in an ethereal solvent such as diethyl ether or tetrahydrofuran or the like to provide amines that can be protected as described in Scheme A1. Hydrogenation with palladium catalysis provides an appropriately substituted pyrrolidine (S2, S₃, S₄ are independently alkyl, lower alkyl, aryl, etc.).

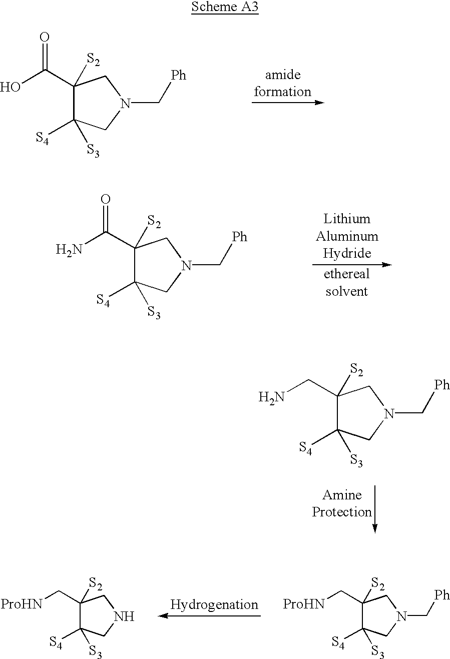

In Scheme A4, a 3-carboxypyrrolidinone [Culbertson T. P., Domagala J. M., Nichols J. B., Priebe S., Skeean R. W., J. Med. Chem., 1987;30(10):1711-1715] can be converted into an acid chloride or Weinreb amide in the same fashion as that defined in Scheme A3. The acid chlorides are reacted with an organocopper reagent or an organo Grignard reagent (for a Weinreb amide) to provide a ketone that can be manipulated as described in Scheme A1 to provide an appropriately substituted pyrrolidine with a variety of substitutions at S₁ (alkyl, lower alkyl, substituted alkyl, aryl). The use of S-methylbenzyl (or R-methylbenzyl) as a protecting group for the pyrrolidine nitrogen allows for the separation of enantiomers and diastereomers at any step in the reaction sequence.

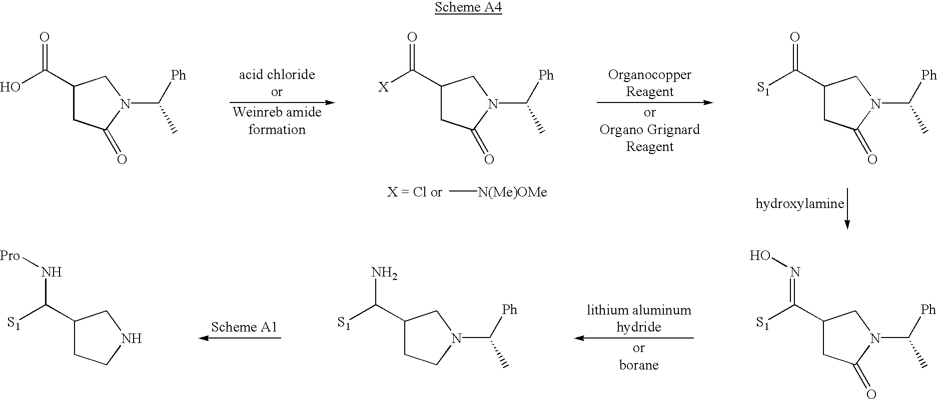

In a particular variant of this approach as provided in Scheme A4.1, the ketone is formed from the Weinreb amide starting compound. Oxime formation and reduction provides the primary amine, and removal of the protecting group provides the target compound

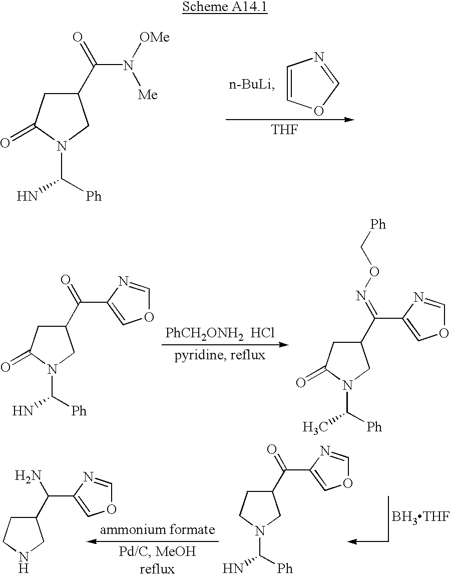

In Scheme A5, the protected dihydropyrrole undergoes [3+2] cycloaddition with the imine oxide generated in situ from hydroxylbenzyl amine to provide the bicyclic intermediate. Removal of the benzyloxycarbonyl group and cleavage of the N—O bond can be effected under hydrogenation conditions to provide the target sidechain.

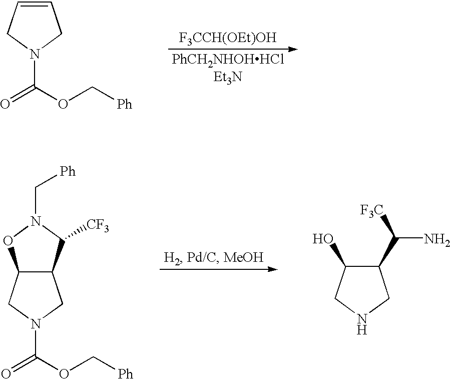

A [3+2] cycloaddition approach is employed as the first step in an approach to a [3.3.0] heterocyclic sidechain as depicted in Scheme A6 starting from the N-benzyl protected dihydropyrrole. Cleavage of the N—O bond of the cycloaddition product using lithium aluminum hydride gives the shown pyrrolidinol derivative. Boc protection of the primary amine, followed by removal of the THP protecting group, gives the shown pyrrolidinyl diol. Conversion of the primary alcohol to a leaving group using DAST and intermolecular cyclization gives the fused bicyclic compound. The benzyl protecting group is then removed.

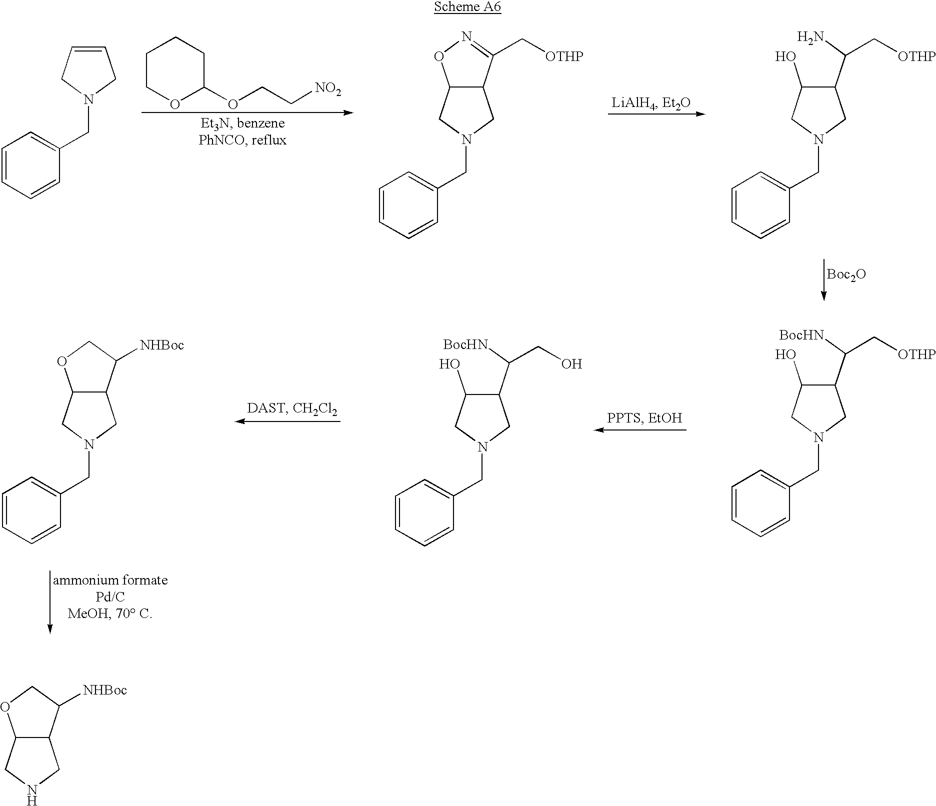

Scheme A7 provides an approach to the synthesis of a sidechain incorporating a thiophene core. Treatment of the thiophene starting compound with bromine in the presence of sodium acetate provides the bromide. The keto moiety is then converted to the benzyl oxime, which is reduced to the primary amine using borane. A chloromethyl group is then attached to the 2-position using HCl in the presence of formaldehyde. Protection of the primary amine, with concomittant cyclization via displacement of the chloride provides the bicyclic compound. Stannylation using butyl lithium, followed by quenching with tri-n-butyltin chloride provides the target compound, ready for coupling to the quinazolinedione core.

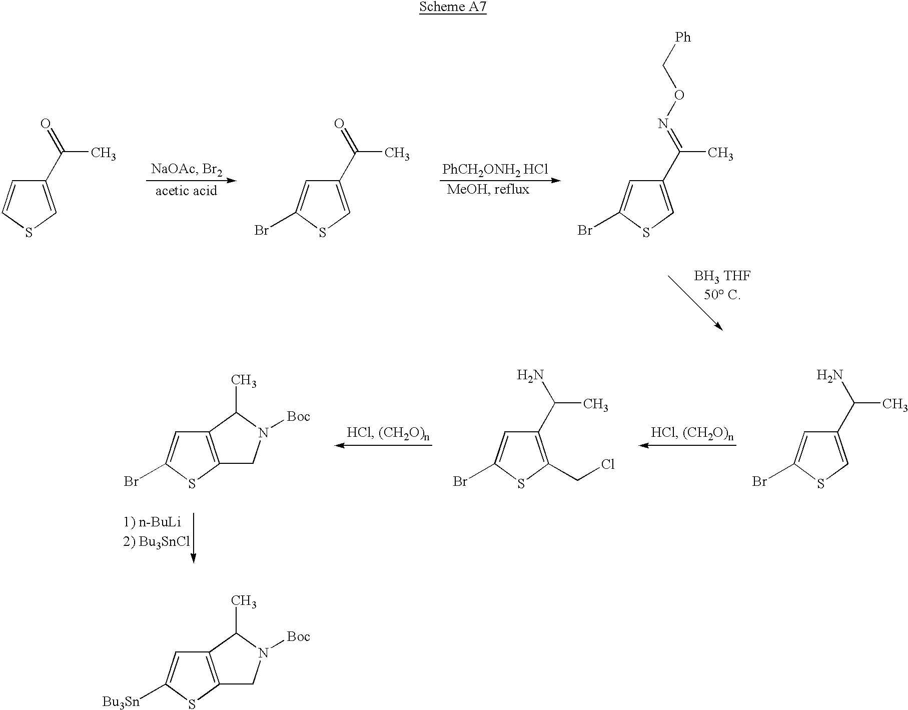

In Scheme A8, 2-thiophenacetonitrile is treated with 1,2 dibromoethane in the presence of base to provide the cyclopropanecarbonitrile. Saponification of the nitrile group with sodium hydroxide provides the cyclopropane carboxylic acid compound. The acid is then converted to the protected amine. Formation of the stannane can be effected using n-butyl lithium and tri-n-butyl stannyl chloride.

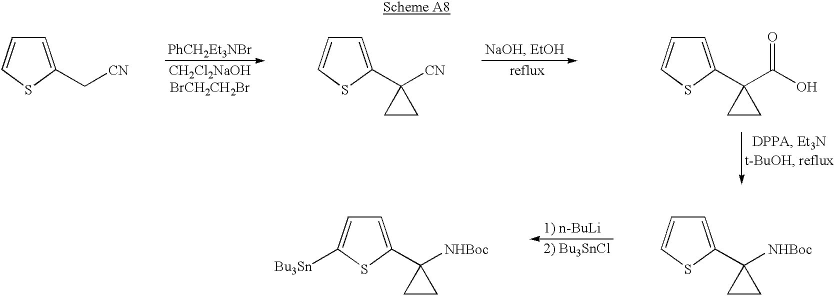

In Scheme A9, the oxime in the fused thiophene starting compound is reduced with borane. The resulting primary amine is then protected, and the resulting protected compound is then converted to the stannane as provided in the previous scheme.

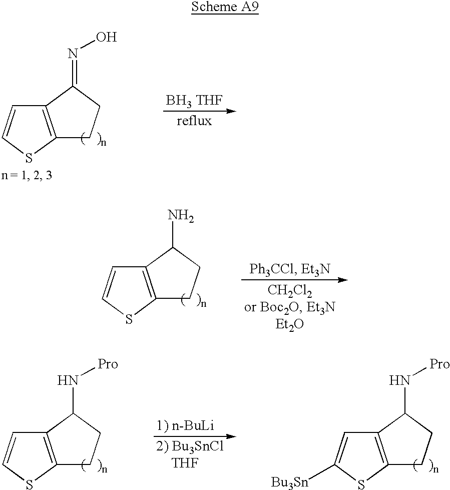

In Scheme A10, the fused thiophen-piperidinyl amine is protected as provided in earlier schemes.

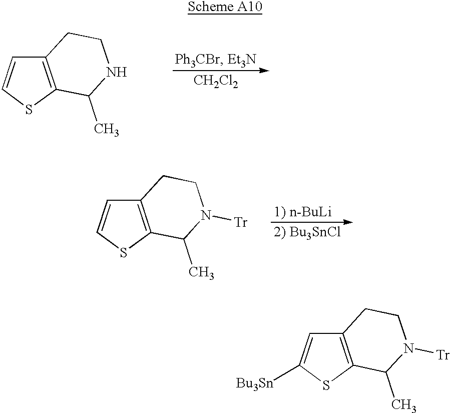

In Scheme A11, the carboxylic acid moiety of the pyrrolidinyl starting compound is converted to a Weinreb amide, which undergoes reaction with cyclopropyl lithium to provide the corresponding ketone. The ketone is converted to a primary amino group via formation of the oxime, and then hydrogenation in the presence of Raney Nickel to provide the target compound.

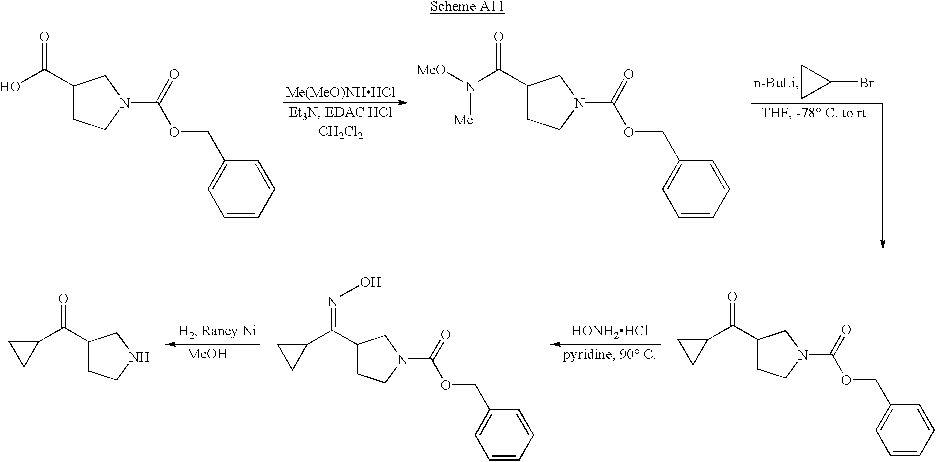

In Scheme A12, the starting compound undergoes reaction with N,N dibenzylacrylamide to provide the shown pyrrolidinyl ketone. Cyclopropanation of the ketone moiety provides the target compound after removal of the protecting groups (Angew. Chemie Int. Ed. Eng. 1996, 35, 413).

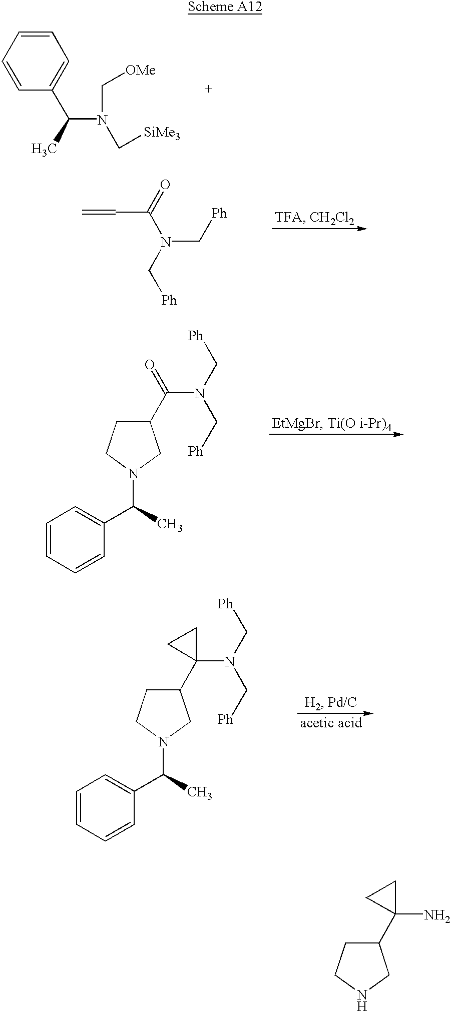

Scheme A12, provides a synthesis for an additional pyrrolidinyl sidechain using techniques known to those skilled in the art. In one approach, the acid moiety in the starting compound is treated with isobutyl chloroformate in the presence of base to form the mixed anhydride. Conversion of the mixed anhydride to the diazomethyl compound using 1-methyl-3-nitro-1-nitrosoguanidine and KOH, followed by treatment with HBr/HOAc, gives rise to α-bromomethyl ketone. The alpha-bromomethyl ketone is readily converted to the fluoromethyl ketone using a fluorine source such as KF. Reductive amination of using benzylamine and a reducing agent such as sodium triacetoxyborohydride provides the fluoroethylaminopyrrolidinone derivative. Reduction of the amide moiety in the pyrrolidinone, followed by hydrogenolysis to remove the benzyl moieties provided the target compound.

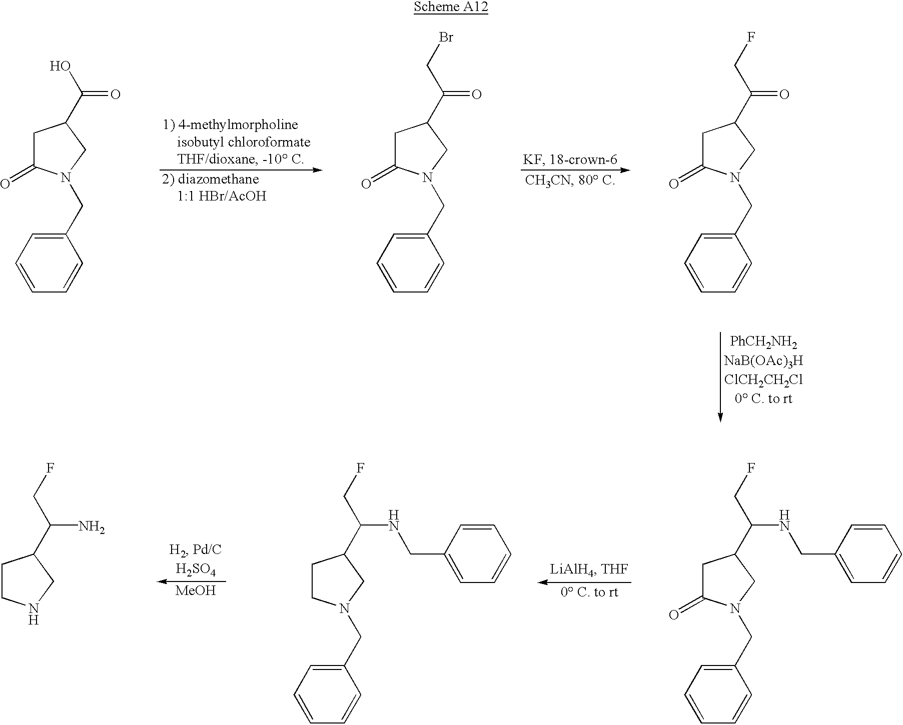

In Scheme A13, the Weinreb amide is prepared from the corresponding carboxylic acid starting compound as provided earlier. Formation of the phenyl ketone from the Weinreb amide is effected via addition of fluorophenyl lithium. Oxime formation, followed by reduction, provides the primary amine. Lithium aluminum hydride reduction of the amide moiety is followed by protection of the primary amine. The pyrrolidinyl nitrogen is then deprotected as provided in Scheme A4 and then is reprotected. A series of purifications and deprotection procedures provides the target compound.

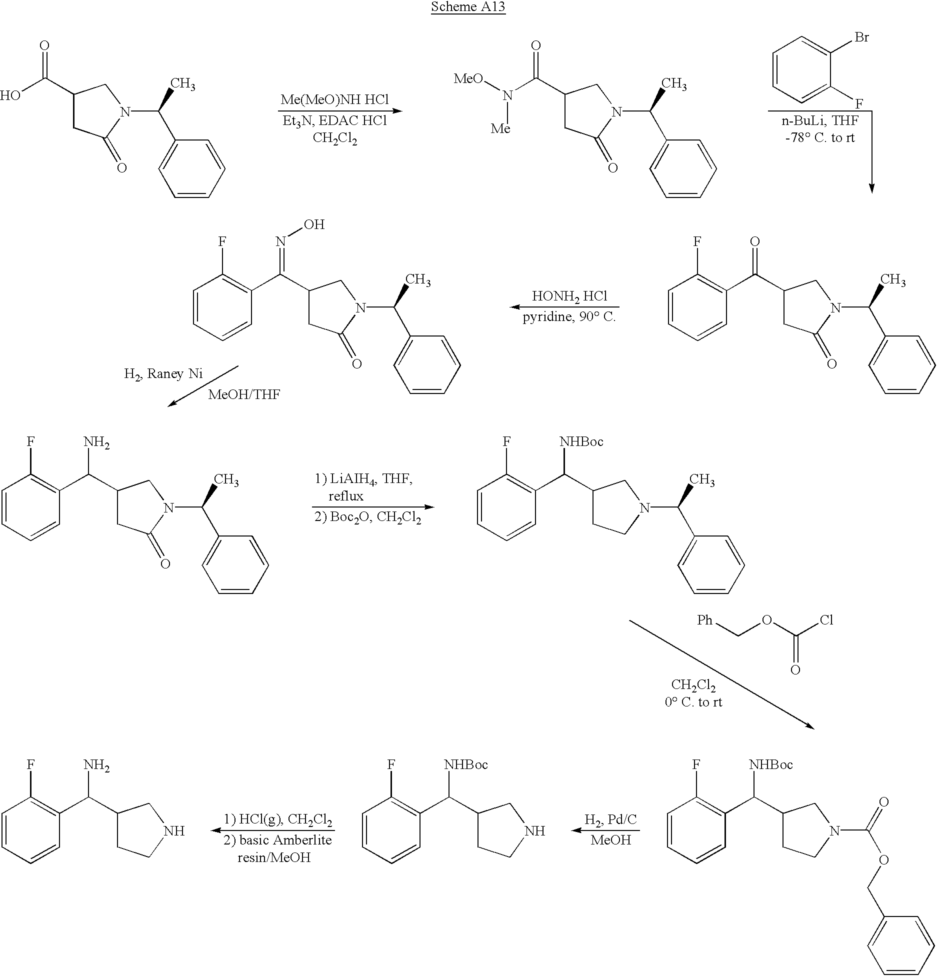

In Scheme A14, the starting ester is reduced to the primary alcohol using sodium borohydride in the presence of lithium chloride. Mesylation of the alcohol, followed by treatment with tetrabutyl ammonium fluoride, provides the primary fluoro compound. Alkylation of the lactone moiety using lithium diisopropyl amide and chloromethyl benzyl ether provides the fluoromethylbenzyl ether as a mixture of diastereomers. The diastereomers are separated, and the benzylether protecting group is selectively removed to afford the primary alcohol. The lactone is then reduced with LAH. Mesylation of the primary alcohol, followed by azide displacement and reduction provides the target compound.

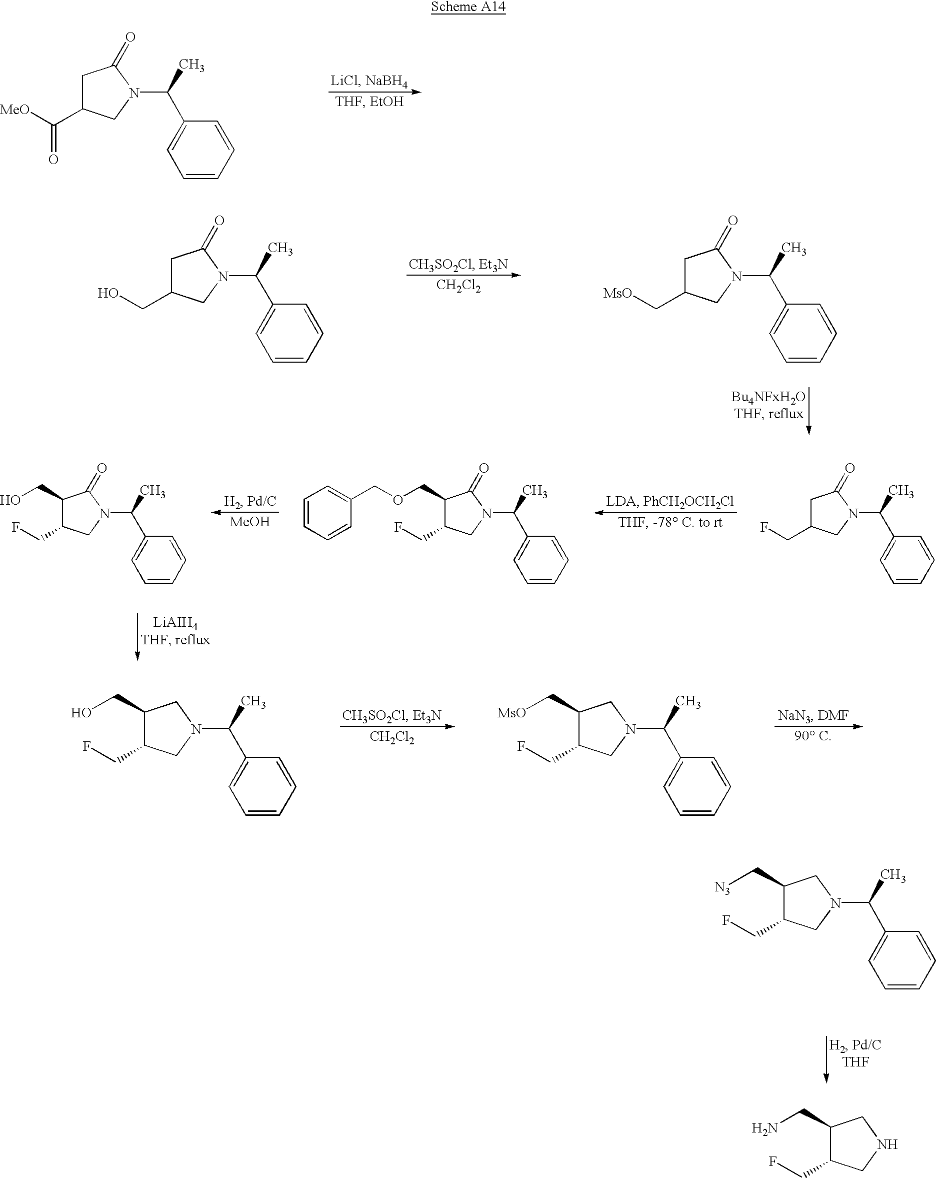

It should be recognized from all of the above schemes that substitutions on ring systems such as a pyrrolidine, piperazine, or piperidine ring will form chiral centers giving R and S enantiomers and diastereomers. Such enantiomers or diastereomers may be separated, if desired, by chiral HPLC at any stage. Resolution of any of the intermediates may be performed with techniques of fractional crystallization using mandelic acid, tartaric acid, or other chiral, optically pure acid resolving agents. Chiral benzylic amines can be used in the preparation of starting materials for the above schemes, and chiral amides may also be prepared using chiral acids, such as mandelic acid and the like. The isomers can then be separated and the chiral amine can be hydrogenated, or the chiral amide can be hydrolyzed.

Some of the compounds of Formula I are capable of further forming pharmaceutically acceptable acid-addition and/or base salts. All of these forms are within the scope of the present invention.

Pharmaceutically acceptable acid addition salts of the compounds of Formula I include salts derived from nontoxic inorganic acids such as hydrochloric, nitric, phosphoric, sulfuric, hydrobromic, hydroiodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinate suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like.

Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds of the present invention possess one or more chiral centers and each center may exist in the R(D) or S(L) configuration. The present invention includes all enantiomeric and epimeric forms, as well as the appropriate mixtures thereof.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally. It will be obvious to those skilled in the art that the following dosage forms may comprise as the active component, either a compound of Formula I or a corresponding pharmaceutically acceptable salt of a compound of Formula I. The formulations typically will comprise from about 1 to about 95 percent by weight of the active invention compound.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection liquid preparations can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or, synthetic gums, resins, methylcellulose, sodium carboxymethylcellulose, and other well-known suspending agents.

Also included are solid form preparations which are intended to be converted, shortly before use, to liquid form preparations for oral administration.

Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is divided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 100 mg preferably 0.5 mg to 100 mg according to the particular application and the potency of the active component as determined by a skilled physician. The composition can, if desired, also contain other compatible therapeutic agents.

In therapeutic use as agents for the treatment of infections caused by a bacteria, the compounds utilized in the pharmaceutical method of this invention are administered at the initial dosage of about 0.01 mg to about 500 mg/kg daily. A daily dose range of about 0.01 mg to about 100 mg/kg is preferred. The dosages, however, may be varied depending upon the requirements of the patient, the severity of the condition being treated, the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

Certain of the compounds of the present invention possess one or more chiral centers and each center may exist in the R or S configuration. The present invention includes all diastereomeric, enantiomeric, and epimeric forms as well as the appropriate mixtures thereof. Additionally, the compounds of the present invention may exist as geometric isomers. The present invention includes all cis, trans, syn, anti, entgegen (E), and zusammen (Z) isomers as well as the appropriate mixtures thereof.

The ability of a compound of the invention to inhibit bacterial growth, demonstrate in vivo activity, and enhanced pharmacokinetics are demonstrated using pharmacological models that are well known to the art, for example, using models such as the tests described below.

Test A-Antibacterial Assay

The compounds of the present invention were tested against an assortment of Gram-negative and Gram-positive organisms using standard microtitration techniques (Cohen, et al., Antimicrob. Agents Chemother., 1985;28:766; Heifetz, et al., Antimicrob. Agents Chemother:, 1974;6:124). The results of the evaluation are shown in Table 3 and are compared to ciprofloxacin.

TABLE 3
Antibacterial and E. coli gyrase Activities

| | Minimum Inhibitory Concentrations μg/mL | | | | | | |
|---|---|---|---|---|---|---|---|
| | Gram Negatives | | | Gram Positives | | | |
| Com- | E. | | | | | | E. coli |
| pound | coli | E. | E. | E. | S. | S. | gyrase |
| Number or | MC | coli | coli | faecalis | aureus | pyogenes | IC₅₀ |
| Structure | 4100 | B90 | Tol C | RB1 | 29213 | C203 | (μM) |
| 2 | 8.0 | 0.5 | 0.25 | 0.25 | 0.5 | 0.03 | 2.1 |
| 3 | 32.0 | 2.0 | 0.5 | 2.0 | | 0.13 | 7.0 |
| 4b | 4.0 | 0.13 | 0.13 | 0.13 | 0.25 | 0.015 | 0.6 |
| 7j | 8.0 | 0.25 | 0.06 | 0.25 | 0.13 | 0.03 | 0.8 |
| 19f | 4.0 | 0.25 | 0.13 | 0.06 | 0.13 | 0.06 | 1.8 |
| 22j | 8.0 | 1.0 | 1.0 | 2.0 | 1.0 | 0.25 | 1.4 |
| 26b | 2.0 | 0.25 | 0.13 | 0.13 | 0.25 | 0.03 | 1.6 |
| 23b | 16.0 | 0.13 | <0.06 | 1.0 | 0.5 | 2.0 | 1.0 |
| 16d | >64.0 | 16.0 | 4.0 | 1.0 | 1.0 | 0.5 | 25.0 |
| 41c | 2.0 | 0.25 | 0.06 | 0.03 | 0.03 | 0.008 | 0.6 |
| Cipro- | <0.02 | <0.01 | <0.01 | 0.5 | 0.5 | 0.5 | 0.2 |
| floxacin | | | | | | | |

Test B-DNA Gyrase Assay

The effects of test agents on the activity of DNA gyrase was determined by the supercoiling inhibition assay, following reaction conditions recommended by the enzyme supplier (Lucent, Ltd., Leicester, UK), as follows: Reactions are performed in buffer G (35 mM Tris-HCl (pH 7.5), 24 mM KCl, 4 mM MgCl₂, 2 mM DTT, 1.8 mM spermidine, 1 mM ATP, 0.1 mg/mL bovine serum albumin). Relaxed plasmid pBR322 (0.25 μg, Lucent, Ltd., Leicester, UK) is reacted with 1 U E. coli gyrase (Lucent, Ltd., Leicester, UK), in the absence or presence of drugs, for 30 minutes at 37° C. Reactions were stopped by the addition of SDS and proteinase K to respective final concentrations of 1% and 0.5 mg/mL. After an additional 30 minutes at 37° C., one-tenth volume of 10× loading buffer (0.3% bromophenol blue, 16% Ficoll, 10 mM Na₂HPO₄) was added, and reactions were loaded onto agarose gels and electrophoresed as described for intercalation assays (Y. Pommier et al. Nucleic Acids Research 1987, 15, 6713-6731.). The concentration of drug inhibiting 50% of the supercoiling activity of DNA gyrase was measured and is given as an IC₅₀ in Table 3.

Test C-In Vivo Activity (Mouse)

The in vivo activity was obtained when the compounds were tested according to the procedure of Miller, et al. (Proc. Soc. Exp. Biol. Med., 1944;57:261). The median protective dose (PD₅₀) was determined in mice given lethal systemic infections, as depicted in Table 4. Compounds 2 and 4b are compared to ciprofloxacin.

TABLE 4
In Vivo Median Protective Dose (PD₅₀) in Mice (PO)

| Compound Number or | | |
|---|---|---|
| Structure | Organism | PD₅₀ (mg/kg) |
| 2 | S. pyogenes | 10.8 |
| 4b | S. pyogenes | 3.6 |
| Ciprofloxacin | S. pyogenes | >100.0 |

Test D-Cross Resistance Antibacterial Assay

The compounds of the present invention were tested against an assortment of ciprofloxacin resistant E. coli and S. aureus organisms described below using standard microtitration techniques (Cohen, et al., Antimicrob. Agents Chemother., 1985;28:766; Heifetz, et al., Antimicrob. Agents Chemother., 1974;6:124). The results of the evaluation are shown in Table 5 compared to ciprofloxacin.

N. gonorrhoeae and S. aureus Organisms:

N. gonorrhoeae 2637 (N.g. 2637) is a derivative of Neisseria gonorrhoeae MS11 containing a TAC-LAC recA to allow for control of homologous recombination [Tonjum T. et al. Molecular Microbiology 1995, 16, 451-64].

N. gonorrhoeae 2709 (N.g. 2709): Isogenic to N. gonorrhoeae 2637 contains gyrA quinolone-resistant determining region (QRDR) mutations (S91F D95G).

N. gonorrhoeae 2693 (N.g. 2693): Isogenic to N. gonorrhoeae 2709 containing parC QRDR mutations [P88S and E91K].

S. aureus UC-76: Typical sensitive laboratory strain (Wild type).

S. aureus 2552: Isogenic to S. aureus UC-76, with upregulated norA pump.

S. aureus 2554: Isogenic to S. aureus 2552, with point mutation at position 80 of grlA subunit.

S. aureus 2558: Isogenic to S. aureus 2554, with point mutation at position 84 of gyrA subunit.

TABLE 5
Antibacterial Activities Against Ciprofloxacin Resistant Strains

| Com- | Minimum Inhibitory Concentrations μg/mL | | | | | | |
|---|---|---|---|---|---|---|---|
| pound | | | | S. | S. | S. | S. |
| Number or | N. g. | N. g. | N. g. | aureus | aureus | aureus | aureus |
| Structure | 2637 | 2709 | 2693 | UC-76 | 2552 | 2554 | 2558 |
| 2 | 0.5 | 4.0 | 8.0 | 0.25 | 0.5 | 0.5 | 1.0 |
| | | (8x) | (16x) | | (2x) | (2x) | (4x) |
| 4b | 0.25 | 2.0 | 2.0 | 0.13 | 0.5 | 0.25 | 1.0 |
| | | (8x) | (8x) | | (4x) | (2x) | (8x) |
| 7j | 0.13 | 0.5 | 1.0 | 0.06 | 0.06 | 0.06 | 0.06 |
| | | (4x) | (8x) | | (1x) | (1x) | (1x) |
| Cipro- | 0.002 | 0.06 | 2.0 | 0.13 | 2 | 2 | 64 |
| floxacin | | (30x) | (1000x) | | (15x) | (15x) | (123x) |

Test E-Pharmacokinetic Behavior or Quinazolinediones vs. 3-Aminoquinazolinediones

The compounds of the present invention were tested for pharmacokinetic behavior against the structurally related 3-aminoquinazolinediones in rats, dogs and monkeys. The representative result of the evaluation for compound 4b is shown in Table 6 compared to a structurally similar 3-aminoquinazolinedione.

Male Wistar Rats

In this study, compounds were administered to male Wistar rats and were dosed at 1 mg/kgIV infusion in D5W over 5 minutes. Blood samples were drawn at 0, 0.083, 0.167, 0.25, 0.5, 1, 2, 4, 6, 8, 12, and 24 hours post dose.

Male Beagle Dogs

Male Beagle Dogs were dosed with compound dissolved in D5W at 5 mg/kg IV Infusion over 15 minutes. Blood samples will be collected at 0, 0.167, 0.25, 0.33, 0.5, 1, 2, 4, 6, 8, 12 and 24 hours post dose.

In Monkeys

Male Cynomologous Monkeys were dosed with compound dissolved in D5W at 5 mg/kg IV Infusion over 15 minutes. Blood samples were collected at 0, 0.167, 0.25, 0.33, 0.5, 1, 2, 4, 6, 8, 12 and 24 hours post dose.

Plasma from all samples were harvested following centrifugaltion and stored frozen until plasma concentrations were determined using liquid chromatography/mass spectroscopy methods. The concentration/time profile for each compound in each animal species was analyzed using non-compartmental pharmacokinetic analysis approach. In the table, clearance is defined as the volume of fluid cleared of drug from the body per unit of time. Clearance is a quantitative assessment of drug elimination. Drug elimination is the irreversible removal of drug from the body by all routes of elimination.

TABLE 6
Comparative Pharmacokinetic Behavior of quinazolinediones and 3-
Aminoquinazolinediones in Rats, Dogs and Monkeys

| | Pharmacokinetic | | | |
|---|---|---|---|---|
| | parameters | Rat | Dog | Monkey |
| 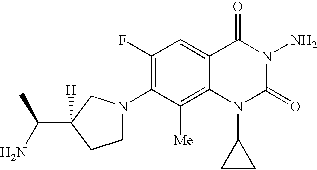 | half-life (hours) Clearance (mL/min/kg) | 2 62 | 2.4 21 | 2.2 24 |
| 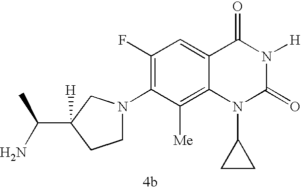 | half-life (hours) Clearance (mL/min/kg) | 1.9 47 | 6.3 8.7 | 4.1 17 |

The antibacterial agents described in this invention display Gram-negative and Gram-positive activity. The compounds also show inhibition of bacterial DNA gyrase.

Finally, the compounds demonstrate in vivo protective activity in mice and are not highly cytotoxic to mammalian cells indicating selectivity for bacteria.

Representative examples of methods for preparing compounds of the invention are set forth below.

### EXAMPLE 1

a) 2-Amino-4,5-difluoro-N-methoxybenzamide

4,5-Difluoroanthranilic acid (5.0 g, 20.0 mmol) and carbonyl dilmidazole (5.63 g, 32.0 mmol) were combined in 200 mL of dry tetrahydrofuran and heated to reflux for 8 hours. O-methylhydroxylamine hydrochloride (2.42 g, 20.0 mmol) and triethylamine (4.95 mL, 35.0 mmol) were added to the cooled mixture and it was returned to reflux for 18 hours. The mixture was cooled and concentrated to give a solid. The solid was dissolved in chloroform and washed with 1 N hydrochloric acid, saturated sodium bicarbonate and brine. The organic layer was dried over magnesium sulfate and concentrated to afford 3.96 g of a solid. The solid was purified by chromatography (SiO₂, chloroform to Chloroform/methanol 95/5) to afford 2.5 g of the title compound. ¹H NMR (400 MHz, d₆-DMSO) δ 11.45 (bs, 1H), 7.36 (dd, 1H), 6.65 (dd, 1H), 6.49 (bs, 2H), 3.65 (s, 3H), MSCI: m/z=203 (MH⁺).

b) 6, 7-Difluoro-3-methoxy-1H-quinazoline-2,4-dione

A 12.5% solution of phosgene in toluene (4.6 mL, 5.3 mmol) was added to a solution of 2-amino-4,5-difluoro-N-methoxybenzamide (1.07 g, 5.3 mmol, Example 1a) in 30 mL of dioxane. The solution was heated at reflux for 20 hours, then poured into 100 mL of water. The aqueous mixture was extracted with ethyl acetate and the combined organic layers were washed with water, brine and dried over magnesium sulfate. The solution was concentrated to give 1.16 g of the title compound. H¹ NMR (400 MHz, CDCl₃) δ 11.50 (bs, 1H), 7.80 (m, 1H), 6.99 (m, 1H), 3.97 (s, 3H); MSCI: m/z=229 (MH⁺).

c) 6,7-Difluoro-3-methoxy-1-methylcyclopropyl-1H-quinazolinedione

A solution of 6,7-difluoro-3-methoxy-1H-quinazolinedione (1.15 g, 5.0 mmol, Example 1b) in N,N-dimethylformamide (20 mL) was added to a suspension of sodium hydride (0.24 g, 6.0 mmol) in N,N-dimethylformamide (15 mL) and stirred for 45 minutes. Bromomethylcyclopropane (0.73 mL, 7.6 mmol) was added and the mixture stirred at 25° C. for 18 hours. The reaction was quenched with water (1 mL) and concentrated to provide an oil that was dissolved in chloroform. The solution was washed with water, brine, dried over magnesium sulfate and concentrated to a solid that was purified by flash silica gel chromatography (chloroform then 97:3 chloroform:methanol) to afford the title compound (0.86 g). ¹HNMR (400 MHz, CDCl₃) δ 8.06 (t, 1H), 7.15 (dd, 1H), 4.05 (s, 3H), 4.01 (d, 2H), 1.23-1.15 (m, 1H), 0.62-0.51 (m, 4H); MSCI: m/z=283 (MH⁺).

d) 3-(1-Cyclopropylmethyl-6-fluoro-3-methoxydioxo-1,2,3,4-tetrahydro-quinazolin-7-yl)-3-azabicyclo[3.1.0]hex-6-yl]carbamic Acid Tert-Butyl Ester (1α, 5α, 6α)

(3-Azabicyclo[3.1.0]hex-6-yl)carbamic acid tert-butyl ester(1α, 5α, 6α) (0.16 g, 7.8 mmol, [Eur. Pat. Appl. EP 413455 A2]) was added to a solution of 6,7-difluoro-]-methylcyclopropyl-3-methoxy-1H-quinazoline-2,4-dione (0.15 g, 5.3 mmol, Example 1c) and triethylamine (0.12 mL, 0.9 mmol) in acetonitrile (15 mL). The solution was heated at reflux for 17 hours, cooled, and concentrated to a solid. The solid was dissolved in chloroform, and the resulting solution was washed successively with 1N hydrochloric acid, saturated sodium bicarbonate, and brine. The solution was dried over magnesium sulfate and concentrated to give a solid that was purified by flash silica gel chromatography (chloroform then 97:3 chloroform:methanol) to afford the title compound (0.18 g): ¹H NMR (400 MHz, CDCl₃) δ 7.43 (d, 1H), 6.24 (d, 1H), 4.77 (bs, 1H), 4.05-3.83 (m, 7H), 3.68-3.58 (m, 2H), 2.44 (s, 1H), 1.91 (s, 2H), 1.45 (s, 9H), 1.23-1.15 (m, 1H), 0.62-0.51 (m, 4H); MSCI: m/z=461 (MH⁺).

e) 3-(1-Cyclopropylmethyl-6-fluorodioxo-1,2,3,4-tetrahydroquinazolin-7-yl)-3 azabicyclo[3.1.0]hex-6-yl]carbamic Acid Tert-Butyl Ester (1α, 5α, 6α)

3-(1-Cyclopropylmethyl-6-fluoro-3-methoxydioxo-1,2,3,4-tetrahydroquinazolin-7-yl)-3-azabicyclo[3.1.0]hex-6-yl]carbamic acid tert-butyl ester (1α, 5α, 6α) (0.16 g, 0.34 mmol, Example 1d) in methanol (25 mL) was treated with Raney nickel (1 g) and placed under 50 pounds per square inch (psi) of hydrogen for 96 hours at room temperature. The mixture was filtered and concentrated to afford the title compound (0.11 g): ¹H NMR (CDCl₃) δ 7.93 (bs, 1H), 7.66 (d, 1H), 6.26 (d, 1H), 4.78 (bs, 1H), 4.01-3.90 (m, 4H), 3.72-3.55 (m, 2H), 3.13-3.06 (m, 1H), 2.55-2.40 (m, 1H), 1.45 (s, 9H), 1.23-1.15 (m, 1H), 0.62-0.51 (m, 4H); MSCI: m/z=431 (MH⁺).

f) 7-(6-amino-3-aza-bicyclo[3.1.0]hex-3-yl)-6-fluoro-3H-1-methylcyclopropyl-1H-quinazoline-2,4-dione (1α, 5α, 6α) hydrochloride Hydrogen chloride gas was bubbled into a solution of 3-(1-cyclopropylmethyl-6-fluorodioxo-1,2,3,4-tetrahydroquinazolin-7-yl)-3-azabicyclo[3.1.0]hex-6-yl]carbamic Acid Tert-Butyl Ester (1α, 5α, 6α) (0.05 g, 1.1 mmol, Example 1e) in dichloromethane (30 mL) at 0° C. The suspension was stirred for 2 hours and filtered to afford the title compound (0.04 g); mp>250° C.: ¹H NMR (DMSO-d₆) δ 11.30 (bs, 1H), 8.37 (bs, 3H), 7.49 (d, 1H), 6.41 (d, 2H), 3.96 (d, 2H), 3.85-3.77 (m, 2H), 3.60-3.55 (m, 2H), 2.13 (s, 2H), 1.25-1.15 (m, 1H), 0.50-0.46 (m, 2H), 0.45-0.35 (m, 2H); MSCI: m/z 331 (MH⁺).

### EXAMPLE 2

1-Cyclopropyl-6-fluoro-8-methyl-7-[(R)-3-((S)-1-methylaminoethyl)pyrrolidin-1-yl]-1H-quinazolinedione

1-Cyclopropyl-6,7-difluoro-8-methyl-1H-quinazolinedione (0.2 g, 0.79 mmol, [PCT Int. Appl. WO 0153273 A1]) and methyl-((R)-(S)-1-pyrrolidinyl-3-ylethyl)amine (0.31 g, 2.4 mmol, [J. Het. Chem., 1992, 29,1481]) in dimethyl sulfoxide (1 mL) was heated at 80° C. for 6 hours. The solution was diluted with water (4 mL) and saturated ammonium chloride (1.5 mL) and stirred for 2 hours. The mixture was filtered and dried to afford the title compound (0.23 g); mp>250° C.: ¹H NMR (CDCl₃) δ 7.33 (d, 1H), 3.60-3.50 (m, 1H), 3.45-3.20 (m, 4H), 3.16-3.08 (m, 1H), 2.55-2.38 (m, 5H), 2.35 (s, 3H), 2.60-2.00 (m, 1H), 1.72-1.64 (m, 1H), 1.21 (d, 3H), 1.05-0.95 (m, 2H), 0.58-0.42 (m, 2H); MSCI: m/z 361 (MH⁺).

### EXAMPLE 3

1-Cyclopropyl-6-fluoro-8-methoxy-7-[(R)-3-((S)-1-methylaminoethyl)pyrrolidin-1-yl]-1H-quinazolinedione

Utilizing the same procedure as described in Example 2, from reaction of 1-cyclopropyl-6,7-difluoro-8-methoxy-1H-quinazolinedione [PCT Int. Appl. WO 0153273 A1 A1] and methyl-((R)-(S)-1-pyrrolidinyl-3-ylethyl)amine; mp 162-164° C.: ¹H NMR (CDCl₃) δ 7.23 (d, 1H), 3.73-3.61 (m, 1H), 3.58-3.24 (m, 8H), 3.17-3.10 (m, 1H), 2.27 (s, 3H), 2.16-2.04 (m, 1H), 2.00-1.92 (m, 1H), 1.62-1.50 (m, 1H), 1.03 (d, 3H), 1.05-0.95 (m, 1H), 0.86-0.78 (m, 1H), 0.60-0.52 (m, 1H), 0.50-0.42 (m, 1H); MSCI: m/z=377 (MH⁺).

### EXAMPLE 4

a) {(S)-1-[(R)-1-(1-Cyclopropyl-6-fluoro-8-methyldioxo-1,2,3,4-tetrahydro-quinazolin-7-yl)pyrrolidin-3-yl]ethyl}carbamic Acid Tert-Butyl Ester

1-Cyclopropyl-6,7-difluoro-8-methyl-1H-quinazolinedione (1.5 g, 6.0 mmol) and (R)-(S)-1-pyrrolidin-3-ylethyl)carbamic acid tert-butyl ester (1.78 g, 15.4 mmol, [J. Het. Chem. 1992, 29, 481]) in dimethyl sulfoxide (5 mL) was heated at 90° C. for 10 days. The solution was diluted with water (16 mL) and saturated ammonium chloride (4 mL) and stirred for 2 hours. The solid was collected by filtration, dried and purified by flash silica gel chromatography (chloroform then 98:2 chloroform/methanol) to afford the title compound (0.71 g) as a white solid: ¹H NMR (CDCl₃) δ 8.13 (bs, 1H), 7.52 (d, 1H), 4.48 (d, 1H), 3.80-3.70 (m, 1H), 3.68-3.59 (m, 1H), 3.51-3.43 (m, 1H), 3.44-3.37 (m, 2H), 3.36-3.29 (m, 1H), 2.39 (s, 3H), 2.32-2.23 (m, 1H), 2.12-2.04 (m, 1H), 1.78-1.66 (m, 1H), 1.43 (s, 9H), 1.21 (d, 3H), 1.23-1.17 (m, 1H), 1.12-1.04 (m, 1H), 0.64-0.56 (m, 2H); MSCI: m/z 447 (MH⁺).

b) 7-[(R)-3-((S)-1-Aminoethyl)pyrrolidin-1-yl]-1-cyclopropyl-6-fluoro-8-methyl-1H-quinazolinedione Hydrochloride

Hydrogen chloride gas was bubbled into a solution of {(S)-1-[(R)-1-(1-cyclopropyl-6-fluoro-8-methyldioxo-1,2,3,4-tetrahydroquinazolin-7-yl)pyrrolidin-3-yl]ethyl}carbamic acid tert-butyl ester (0.69 g, 1.5 mmol, Example 4a) in dichloromethane (30 mL) at 0° C. The suspension was stirred for 2 hours and filtered to afford the title compound (0.48 g); mp 200-202° C.: ¹H NMR (DMSO-d₆) δ 11.28 (s, 1H), 7.84 (bs, 3H), 7.35 (d, 1H), 3.60-3.50 (m, 1H), 3.50-3.20 (m, 4H) 2.40-2.22 (m, 4H), 2.10-2.02 (m, 1H), 1.75-1.65 (m, 1H), 1.24 (d, 3H), 1.05-0.98 (m, 2H), 0.55-0.45 (m, 2H); MSCI: m/z 347 (MH⁺).

### EXAMPLE 5

1-Cyclopropyl-7-dimethylamino-6-fluoro-8-methyl-1H-quinazolinedione

1-Cyclopropyl-6,7-difluoro-8-methyl-1H-quinazolinedione (1.5 g, 6.0 mmol) in N,N-dimethylformamide (5 mL) was heated at 90° C. for 10 days. The solution was diluted with water (16 mL) and saturated ammonium chloride (4 mL), and stirred for 2 hours. The solid was collected by filtration, dried, and purified by flash silica gel chromatography (chloroform then 98:2 chloroform/methanol) to afford the title compound (0.66 g); mp 238-239° C.; ¹H NMR (CDCl₃) δ 8.02 (bs, 1H), 7.56 (d, 1H), 3.36-3.31 (m, 1H), 2.96 (d, 6H), 2.47 (s, 3H), 1.16-1.10 (m, 2H), 0.64-0.60 (m, 2H); MSCI: m/z 278 (MH⁺).

### EXAMPLE 6

a) 4-((S)-3-tert-Butoxycarbonylaminopyrrolidin-1-yl)-3-chloro-2-(1-cyclopropyl-ureido)-5-fluorobenzoic Acid Ethyl Ester

To a solution of 4-((S)-3-tert-butoxycarbonylaminopyrrolidin-1-yl)-3-chloro-2-cyclopropylamino-5-fluorobenzoic acid ethyl ester (0.36 g, 0.82 mmol, [PCT Int. Appl. WO 0153273 A1]) in dichloromethane (10 mL), under a nitrogen atmosphere, at 0° C. was added chlorosulfonyl isocyanate (0.14 mL, 1.63 mmol) dropwise via syringe. After 1.5 hours, the reaction mixture was diluted with dichlromethane, washed with saturated sodium bicarbonate, water, and brine. The organic layer was dried over magnesium sulfate, filtered, and filtrate concentrated to afford 4-((S)-3-tert-butoxycarbonyl aminopyrrolidin-1-yl)-3-chloro-2-(1-cyclopropylureido)-5-fluorobenzoic acid ethyl ester (0.323 g) as an oil. MSCI: m/z =485 (MH⁺).

b) [(S)-1-(8-Chloro-1-cyclopropyl-6-fluorodioxo-1,2,3,4-tetrahydroquinazolin-7-yl)pyrrolidin-3-yl]carbamic Acid Tert-Butyl Ester

A solution of 4-((S)-3-tert-butoxycarbonylaminopyrrolidin-1-yl)-3-chloro-2-(1-cyclopropylureido)-5-fluorobenzoic acid ethyl ester (0.323 g, 0.66 mmol, Example 6a) in toluene (10 mL) was refluxed for 24 hours. The reaction mixture was concentrated and the resulting residue purified by flash silica gel chromatography (1:1 ethyl acetate/hexanes) to afford the title compound (0.056 g) as a white solid: MSCI: m/z 439 (MH⁺).

c) 7-((S)-3-Aminopyrrolidin-1-yl)-8-chloro-1-cyclopropyl-6-fluoro-1H-quinazolinedione Trifluoroacetate

To a solution of [(S)-1-(8-chloro-1-cyclopropyl-6-fluorodioxo-1,2,3,4-tetrahydroquinazolin-7-yl)pyrrolidin-3-yl]carbamic acid tert-butyl ester (0.047 g, 0.11 mmol, Example 6b) in dichloromethane (1 mL) was added trifluoroacetic acid (1 mL). After 30 minutes, the precipitate was collected by filtration, washed with hexanes, and dried to afford the title compound (0.031 g); mp 117-118° C.: MSCI: m/z 339 (MH⁺).

### EXAMPLE 7

a) 5-Oxo-1-((S)-1-phenylethyl)pyrrolidine-3-carboxylic Acid Methoxymethylamide

To a solution of 5-oxo-1-((S)-1-phenylethyl)pyrrolidine-3-carboxylic acid (20.4 g, 87.5 mmol, [J. Het. Chem. 1992, 29, 1481]) in dichloromethane (200 mL) at 0° C. was added triethylamine (18.3 mL, 131 mmol), N,O-dimethylhydroxylamine hydrochloride (10.2 g, 105 mmol), and N-(3-dimethylaminopropyl)-N′-ethylcarbodiimide hydrochloride (20 g, 105 mmol). The reaction mixture was slowly warmed to room temperature. After 20 hours, the organic solution was washed with saturated sodium bicarbonate, water, and brine. The organic layer was dried over magnesium sulfate and concentrated. The resulting residue was purified by flash silica gel chromatography (5:95 isopropanol: dichloromethane) to afford the title compound (22.2 g) as a clear oil: MSCI: m/z 246 (MH⁺).

b) 4-[1-(2-Fluorophenyl)methanoyl]-1-((S)-1-phenylethyl)pyrrolidin-2-one

To a solution of 1-bromo-2-fluorobenzene (7.68 g, 43.8 mmol) in tetrahydrofuran (100 mL) under nitrogen atmosphere at −78° C. was added n-butyllithium (1.6 M in hexanes, 30.2 mL, 48.2 mmol) slowly over 30 minutes. After 1 hour, 5-oxo-1-((S)-1-phenylethyl)pyrrolidine-3-carboxylic acid methoxymethylamide (9.68 g, 35.1 mmol, Example 7a) was added via cannula, as a solution in tetrahydrofuran (25 mL). After 30 minutes, the reaction mixture was warmed to room temperature for 1 hour. The reaction mixture was diluted with ethyl acetate and washed with saturated ammonium chloride, water and brine. The organic layer was dried over magnesium sulfate and concentrated to afford the title compound (9.70 g) as a clear brown oil: MSCI: m/z 312 (MH⁺).

c) 4-[1-(2-Fluorophenyl)-1-hydroxyiminomethyl]-1-((S)-1-phenylethyl)pyrrolidin-2-one

To a solution of 4-[1-(2-fluorophenyl)methanoyl]-1-((S)-1-phenylethyl)pyrrolidin-2-one (8.86 g, 28.45 mmol, Example 7b) in pyridine (20 mL) was added hydroxylamine hydrochloride (2.57 g, 36.9 mmol). The reaction was heated to 90° C. for 16 hours, and diluted with ethyl acetate. The organic layer was washed twice with 1N hydrochloric acid, water and brine. The organic layer was dried over magnesium sulfate and concentrated. The resulting residue was purified by flash silica gel chromatography (ethyl acetate) to give the title compound (7.35 g) as a brown oil: MSCI: m/z 327 (MH⁺).

d) 4-[1-Amino-1-(2-fluorophenyl)methyl]-1-((S)-1-phenylethyl)pyrrolidin-2-one

To a solution of 4-[1-(2-fluorophenyl)-1-hydroxyiminomethyl]-1-((S)-1-phenylethyl)-pyrrolidin-2-one (7.35 g, 22.5 mmol, Example 7c) in methanol (50 mL) and tetrahydrofuran (50 mL) was added Raney nickel (5 g). Hydrogen was introduced to the reaction mixture at high pressure (48 psi) for 72 hours. The reaction mixture was then filtered through celite, washed with methanol, and the combined filtrate concentrated under vacuum to afford the title compound (6.23 g) as a brown oil: MSCI: m/z 313 (MH⁺).

e) {1-(2-Fluorophenyl)-1-[1-((S)-1-phenylethyl)pyrrolidin-3-yl]methyl}carbamic Acid Tert Butyl Ester

To a solution of 4-[1-amino-1-(2-fluorophenyl)methyl]-1-((S)-1-phenylethyl)pyrrolidin-2-one (6.23 g, 19.9 mmol, Example 7d) in tetrahydrofuran (25 mL) was added lithium aluminum hydride (1 M in tetrahydrofuran, 39 mL). The reaction mixture was refluxed for 4 hours, cooled to room temperature, and quenched with saturated ammonium chloride. The mixture was diluted with ethyl acetate, washed with saturated ammonium chloride, water, and brine. The organic layer was dried over magnesium sulfate and concentrated. The resulting residue was dissolved in dichloromethane (25 mL) and while stirring, di-tert-butyl dicarbonate (5.66 g, 25.9 mmol) added. After 1 hour, the reaction mixture was concentrated and the resulting residue purified by flash silica gel chromatography (ethyl acetate) to afford the title compound (10.2 g) as an oil: MSCI: m/z 399 (MH⁺).

f) 3-[1-tert-Butoxycarbonylamino-1-(2-fluorophenyl)methyl]pyrrolidine-1-carboxylic Acid Benzyl Ester

To a solution of {1-(2-fluorophenyl)-1-[1-((S)-1-phenylethyl)pyrrolidin-3-yl]methyl}-carbamic acid tert butyl ester (5.65 g, 14.2 mmol, Example 7e) in dichloromethane (50 mL) at 0° C. was added benzyl chloroformate (3.04 mL, 21.3 mmol). The reaction mixture was heated to reflux for 3 hours, cooled to room temperature, and concentrated. The resulting residue was purified by flash silica gel chromatography (1:1 ethyl acetate/hexanes) to give the title compound (7.5 g) as an oil: MSCI: m/z 429 (MH⁺).

g) [1-(2-Fluorophenyl)-1-pyrrolidin-3-ylmethyl]carbamic Acid Tert-Butyl Ester

To a solution of 3-[1-tert-butoxycarbonylamino-1-(2-fluorophenyl) methyl]pyrrolidine-1-carboxylic acid benzyl ester (7.50 g, 17.5 mmol, Example 7f) in methanol (50 mL) was added 20% Pd/C (0.5 g). Hydrogen was introduced to the reaction mixture at high pressure (48 psi) for 72 hours. The reaction mixture was filtered through Celite, washed with methanol, and the combined filtrates concentrated under vacuum to afford the title compound (2.55 g) as an oil: MSCI: m/z 295 (MH⁺).

h) C-[1-(2-Fluorophenyl)-1-pyrrolidin-3-yl]methylamine

To a solution of [1-(2-fluorophenyl)-1-pyrrolidin-3-ylmethyl]carbamic acid tert-butyl ester (1.60 g, 5.43 mmol, Example 7g) in dichloromethane (10 mL) was added a solution of gaseous hydrogen chloride (2M in ether, 10 mL). After 2 hours, the reaction mixture was concentrated and the resulting solid stirred in methanol with Amberlite resin (basic). After 1 hour, the mixture was filtered and the filtrate concentrated to afford the title compound (1.22 g) as an oil: MSCI: m/z 195 (MH⁺).

i) [1-[1-(1-Cyclopropyl-6-fluoro-8-methyldioxo-1,2,3,4-tetrahydroquinazolin-7-yl)pyrrolidin-3-yl]-1-(2-fluorophenyl)methyl]carbamic Acid Tert-Butyl Ester

In a sealed tube, a mixture of 1-cyclopropyl-6,7-difluoro-8-methyl-1H-quinazolinedione (0.350 g, 1.39 mmol), C-[1-(2-fluorophenyl)-1-pyrrolidin-3-yl]methylamine (0.61 g, 3.15 mmol, Example 7h) and triethylamine (0.480 mL, 3.48 mmol) was stirred in dimethyl sulfoxide (1.0 mL) at 130° C. After 24 hours, the reaction mixture was cooled to room temperature and di-tert-butyl dicarbonate (1.52 g, 6.95 mmol) added. After 1 hour, the reaction mixture was diluted with ethyl acetate, washed with saturated sodium bicarbonate, water, and brine. The organic layer was dried over magnesium sulfate and concentrated. The resulting residue was purified by flash silica gel chromatography (80:19:1 dichloromethane: isopropanol:triethylamine) to afford the title compound (0.047 g) as a glassy solid: MSCI: m/z 527 (MH⁺).

j) 7-(3-[1-Amino-1-(2-fluorophenyl)methyl]pyrrolidin-1-yl}-1-cyclopropyl-6-fluoro-6-methyl-1H-quinazolinedione Hydrochloride

To a solution of [1-[1-(1-cyclopropyl-6-fluoro-8-methyldioxo-1,2,3,4-tetrahydroquinazolin-7-yl)pyrrolidin-3-yl]-1-(2-fluorophenyl)methyl]carbamic acid tert-butyl ester (0.037 g, 0.071 mmol, Example 7i) in dichloromethane (1 mL) was added a hydrogen chloride solution (2M in ether, 1 mL). After 16 hours, the precipitate was collected by filtration, washed with hexanes, and dried to afford the title compound (0.019 g); mp 211-213. C: MSCI: m/z 427 (MH⁺).

### EXAMPLE 8

a) 4,6-Dichloro-5-methylnicotinamide

To a solution of 4,6-dichloro-5-methylnicotinic acid (3.0 g, 14.6 mmol, [J. Het. Chem., 1999, 36, 953]) in dichloromethane (50 mL) was added oxalyl chloride (2.5 mL, 29.1 mmol) and dimethylformamide (0.1 mL). After 90 minutes, the reaction mixture was concentrated and the resulting residue redissolved in dichloromethane (25 mL). This solution was slowly added over 10 minutes to a stirred ether solution saturated with gaseous ammonia at 0° C. After 30 minutes, the reaction mixture was allowed to warm to room temperature over a 2 hour period, then concentrated. The solid was triturated in ether/hexanes, collected by filtration, washed with hexanes and dried afford the title compound (3.2 g) as a beige solid: MSCI: m/z 206 (MH⁺).

b) 1-Cyclopropyl-3-[1-(4,6-dichloro-5-methylpyridin-3-yl)methanoyl]urea

To a solution of 4,6-dichloro-5-methylnicotinamide (3.0 g, 14.6 mmol, Example 8a) in 1,2-dichloroethane (50 mL) was added oxalyl chloride (1.91 mL, 22.0 mmol). The reaction mixture was refluxed for 4 hours, cooled to room temperature, and concentrated. The resulting residue was dissolved in dichloromethane and while stirring at 0° C., cyclopropylamine (1.52 mL, 22.0 mmol) was added dropwise over 10 minutes via syringe. After 30 minutes, the reaction mixture was warmed to room temperature and stirred for an additional 16 hours. The reaction mixture was washed with saturated sodium bicarbonate, water, and brine. The organic layer was dried over magnesium sulfate and concentrated. The resulting residue was purified via flash silica gel chromatography (ethyl acetate) to afford the title compound (4.6 g) as a beige solid: MSCI: m/z 289 (MH⁺).

c) 7-Chloro-1-cyclopropyl-8-methyl-1H-pyrido[4,3-d]pyrimidinedione

To a solution of 1-cyclopropyl-3-[1-(4,6-dichloro-5-methylpyridin-3-yl)methanoyl]urea (4.60 g, 15.9 mmol, Example 8b) in tetrahydrofuran (100 mL) at −20° C. was added potassium bis(trimethylsilyl)amide (0.5 M in toluene, 64 mL) dropwise over 20 minutes. After 15 minutes, the reaction mixture was warmed to room temperature and 18-crown-6 ether (0.84 g, 3.18 mmol) added. The reaction mixture was refluxed for 4 hours, cooled to room temperature, and diluted with ethyl acetate. The organic layer was washed with 1N hydrochloric acid, water, and brine. The organic layer was dried over magnesium sulfate and concentrated. The resulting residue was purified by flash silica gel chromatography (ethyl acetate) to give the title compound (2.82 g) as a beige solid: MSCI: m/z 252 (MH⁺).

d) {(S)-1-[(R)-1-(1-Cyclopropyl-8-methyldioxo-1,2,3,4-tetrahydropyrido[4,3-d]pyrimidin-7-yl)pyrrolidin-3-yl]ethyl}methylcarbamic Acid Tert-Butyl Ester

In a sealed tube, 7-chloro-1-cyclopropyl-8-methyl-1H-pyrrido[4,3-d]pyrimidinedione (0.15 g, 0.59 mmol, Example 8c) and methyl-((R)-(S)-1-pyrrolidin-3-ylethyl)amine (0.22 g, 1.79 mmol, [J. Het. Chem. 1992 29,1481]) was stirred in dimethyl sulfoxide (1 mL) at 120° C. After 6 hours, the reaction mixture was cooled to room temperature, and di-tert-butyl dicarbonate (0.65 g, 2.98 mmol) added. After an additional hour, the reaction mixture was diluted with ethyl acetate, washed with saturated sodium bicarbonate, water, and brine. The organic layer was dried over magnesium sulfate and concentrated. The residue was purified via flash silica gel chromatography (ethyl acetate) to afford the title compound (0.177 g) as a clear, glassy solid: MSCI: m/z 444 (MH⁺).

e) 1-Cyclopropyl-8-methyl-7-[(R)-3-((S)-1-methylaminoethyl)pyrrolidin-1-yl]-1H-pyrido[4,3-d]pyrimidinedione Hydrochloride

The reaction of hydrogen chloride with {(S)-1-[(R)-1-(1-cyclopropyl-8-methyldioxo-1,2,3,4-tetrahydropyrido[4,3-d]pyrimidin-7-yl)pyrrolidin-3-yl]ethyl}methylcarbamic acid tert-butyl ester (0.17 g, 0.38 mmol, Example 8d) as described in Example 7j afforded the title compound (0.14 g); mp 217-219° C.: MSCI: m/z 344 (MH⁺).

### EXAMPLE 9

a) [(S)-1-(1-Cyclopropyl-6-fluorodioxo-1,2,3,4-tetrahydropyrido[2,3-d]pyrimidin-7-yl)pyrrolidin-3-yl]carbamic Acid Tert-Butyl Ester

A solution of 1-cyclopropyl-6-fluoro-7-methanesulfanyl-1H-pyrido[2,3-d]pyrimidinedione (0.15 g, 0.5 mmol, [PCT Int. Appl. WO 0153273 A1]), (S)-3-pyrrolidinylcarbamic acid tert-butyl ester (0.28 g, 1.5 mmol, [J. Med. Chem. 1992, 35, 1764]), triethylamine (0.12 mL, 1.5 mmol) and acetonitrile (10 mL) was heated at reflux for 6 hours and then stirred at room temperature for 18 hours. The solution was concentrated and the residue partitioned between ethyl acetate and water. The organic layer was washed with water, dried over magnesium sulfate, and concentrated to a residue that was purified by flash silica gel chromatography (95:5 dichloromethane/ethanol) to afford the title compound (0.17 g); mp 220-222° C.

b) 7-((S)-3-Aminopyrrolidin-1-yl)-1-cyclopropyl-6-fluoro-1H-pyrido[2,3-d]pyrimidinedione Hydrochloride

A solution of [(S)-1-(1-cyclopropyl-6-fluorodioxo-1,2,3,4-tetrahydropyrido[2,3-d]pyrimidin-7-yl)pyrrolidin-3-yl]carbamic acid tert-butyl ester (0.135 g, 0.33 mmol, Example 9a) in ethanol (5 mL) was treated with ethanol (1 mL) saturated with hydrogen chloride gas. The mixture was heated at reflux for 0.5 hours, stirred at room temperature for 18 hours, and then concentrated. The solid was then triturated in ethyl ether, collected by filtration, washed with ether and dried to give the title compound (0.11 g); mp>280° C.

### EXAMPLE 10

a) {(S)-1-[(R)-1-(1-Cyclopropyl-6-fluorodioxo-1,2,3,4-tetrahydropyrido[2,3-d]pyrimidin-7-yl)pyrrolidin-3-yl]ethyl}carbamic Acid Tert-Butyl Ester

Reaction of 1-cyclopropyl-6-fluoro-7-methanesulfanyl-1H-pyrido[2,3-d]pyrimidinedione (0.15 g, 0.5 mmol, [PCT Int. Appl. WO 0153273 A1]) with ((R)-(S)-1-pyrrolidin-3-ylethyl)carbamic acid tert-butyl ester (0.32 g, 1.5 mmol, [J. Het. Chem. 1992, 29, 1481]) as described in Example 9a gives the title compound (0.17 g); mp 249-251° C.

b) 7-[(R)-3-((S)-1-Aminoethyl)pyrrolidin-1-yl]-1-cyclopropyl-6-fluoro-1H-pyrido[2,3-d]pyrimidinedione hydrochloride The reaction of {(S)-1-[(R)-1-(1-cyclopropyl-6-fluorodioxo-1,2,3,4-tetrahydropyrido[2,3-d]pyrimidin-7-yl)pyrrolidin-3-yl]ethyl} carbamic acid tert-butyl ester (0.126 g, 0.29 mmol, Example 10a) with hydrogen chloride as described in Example 9b gave the title compound (0.10 g); mp>280° C.

### EXAMPLE 11

a) 3-Ethylsulfanyl-4,5-difluoro-2-methyl-1H-indole-7-carboxylic Acid Methyl Ester

A solution of 9.5 g (30.7 mmoles) of 7-bromo-3-ethylsulfanyl-4,5-difluoro-2-methyl-11H-indole [Chem. Pharm. Bull. 1990, 38, 2459] in 300 mL of methanol was treated with 0.35 g (1.5 mmol) of palladium acetate, 0.93 g (2.25 mmol) of diphenylphosphinopropane and 7.7 g (10.7 mL, 76 mmol) of triethylamine. The resulting mixture was pressurized to 500 psi with carbon monoxide and heated at 100° C. for 12 hours. The solvent was removed in vacuo and the residue was chromatographed on flash grade silica gel (230-400 mesh) eluting with dichloromethane to give 0.6 g of starting material and 7.2 g of the title compound, mp 110-112° C.

b) 4,5-Difluoro-2-methyl-1H-indole-7-carboxylic Acid Methyl Ester

A solution of 5.2 g (18.2 mmol) of 3-ethylsulfanyl-4,5-difluoro-2-methyl-1H-indole-7-carboxylic acid methyl ester (Example 11a) in 150 mL of ethanol was treated with 30 g of Raney-nickel and the resulting suspension was heated at reflux for 2 hours. An additional 10 g of Raney-nickel was added and the heating was continued for an additional 2 hours. The solid was removed by filtration and washed with ethanol. The ethanolic filtrates were combined and concentrated in vacuo to dryness and used as is for the next step. The yield of the title compound was 4.0 g.

c) 4,5-Difluoro-2-methyl-2,3-dihydro-1H-indole-7-carboxylic Acid Methyl Ester

A suspension of 3.25 g (14.3 mmol) of 4,5-difluoro-2-methyl-1H-indole-7-carboxylic acid, methyl ester (Example 11b) in 50 mL of trifluoroacetic acid was heated to 50° C. and the resulting solution treated dropwise with 3.3 g (4.6 ml, 28.6 mmol) of triethylsilane. The reaction was heated at 50° C. for 3 hours and the solvent removed in vacuo. The residue was then dissolved in methanol, which was also removed in vacuo. The resulting residue was triturated with hexane (2×30 mL) then ether, both of which were removed in vacuo to give 3.1 g of the title compound, mp 50-52° C.

d) 7,8-Difluoro-5-methyl-5,6-dihydro-5H-pyrrolo[3,2,1-ij]quinazoline-1,3-dione

A solution of 3.2 g (14.3 mmol) of 4,5-difluoro-2-methyl-2,3-dihydro-1H-indole-7-carboxylic acid methyl ester (Example 11c) in 60 mL of dichloromethane was treated with 4.06 g (50 mmol) of 96% potassium cyanate. The resulting suspension was stirred at room temperature for 10 minutes and treated with 5.7 g (3.85 mL, 50 mmol) of trifluoroacetic acid. There was a slight exotherm initially. The reaction was stirred at room temperature for 18 hours. A complete solution after 1 hour was followed by the formation of a heavy precipitate. The reaction was concentrated in vacuo and the residue was triturated with ether/water (200 mL of each). The solid was removed by filtration, washed with water, diethyl ether and dried in vacuo to give 3.2 g of the title compound, mp 239-241° C.

e) [(S)-1-(8-Fluoro-5-methyl-1,3-dioxo-2,3,5,6-tetrahydro-1H-pyrrolo[3,2,1-ij]quinazolin-7-yl)pyrrolidin-3-yl]carbamic Acid Tert-Butyl Ester

A solution of 0.12 g (0.5 mmol) of 7,8-difluoro-5-methyl-5,6-dihydro-5H-pyrrolo[3,2,1-i,j]quinazoline-1,3-dione (Example 1d) in 5 mL of dimethyl sulfoxide was treated with 0.37 g (2.0 mmol) of (S)-3-pyrrolidinylcarbamic acid tert-butyl ester [J. Med. Chem. 1992, 35, 1764] and the reaction mixture stirred at room temperature for 40 hours. The reaction was diluted to 50 mL with ice and water and extracted with ethyl acetate (2×40 mL). The combined organics were washed with water (2×30 mL), dried with magnesium sulfate, filtered and evaporated in vacuo. The residue was chromatographed over flash grade silica gel (230-400 mesh) eluting with dichloromethane/ethyl acetate/ethanol (80:20:10) to give 0.19 g of the title compound, mp 203-205° C.

f) 7-((S)-3-Aminopyrrolidin-1-yl)-8-fluoro-5-methyl-5,6-dihydropyrrolo[3,2,0-ij]quinazoline-1,3-dione Hydrochloride

A solution of 0.19 g (0.47 mmol) of [(S)-1-(8-fluoro-5-methyl-1,3-dioxo-2,3,5,6-tetrahydro-1H-pyrrolo[3,2, 1-ij]quinazolin-7-yl)pyrrolidin-3-yl]carbamic acid tert-butyl ester (Example 11e) in 5 mL of ethanol was treated with 1 mL of ethanol saturated with hydrogen chloride gas and the solution stirred at room temperature overnight. The solvent was removed in vacuo and the residue dissolved in water, filtered through a fiber glass pad to clarify and the filtrate lyophilized to give 0.148 g of the title compound, mp 213-215° C.

### EXAMPLE 12

a) [(S)-1-[(R)-1-(8-Fluoro-5-methyl-1,3-dioxo-2,3,5,6-tetrahydro-1H-pyrrolo[3,2,1-ij]quinazolin-7-yl)pyrrolidin-3-yl]ethyl}carbamic Acid Tert-Butyl Ester

A solution of 0.125 g (0.5 mmoles) of 7,8-difluoro-5-methyl-5,6-dihydro-5H-pyrrolo[3,2,1-i,j]quinazoline-1,3-dione (Example 11d), 0.43 g (2.0 mmoles) of ((R)-(S)-1-pyrrolidin-3-ylethyl)carbamic acid tert-butyl ester [J. Het. Chem. 1992, 29, 1481] and 5 mL of dimethyl sulfoxide was heated at 100° C. for 24 hours. The reaction was cooled to room temperature, diluted to 50 mL with ice and water and extracted with ethyl acetate (2×30 mL). The combined organic extracts were washed with water (2×25 mL), dried with magnesium sulfate, filtered and concentrated in vacuo. The residue was chromatographed over flash grade silica gel (230-400 mesh) eluting with dichloromethane/ethanol (90:10) to give 0.19 g of the title compound as a foam which was used “as is” for the next step.

b) 7-[(R)-3-((S)-1-Aminoethyl)pyrrolidin-1-yl)-8-fluoro-5-methyl-5,6-dihydropyrrolo[3,2,1-ij]quinazoline-1,3-dione Hydrochloride

A solution of 0.19 g (0.42 mmol) of [(S)-1-[(R)-1-(8-Fluoro-5-methyl-1,3-dioxo-2,3,5,6-tetrahydro-1H-pyrrolo[3,2,1-ij]quinazolin-7-yl)pyrrolidin-3-yl]ethyl}carbamic acid tert-butyl ester (Example 12a) in 5 mL of ethanol was treated with 2 mL of ethanol saturated with hydrogen chloride gas and the mixture stirred at room temperature for 18 hours. The solvent was removed in vacuo and the residue triturated. with ethyl acetate. The resulting solid was removed by filtration, washed with ethyl acetate and dried in vacuo affording 0.14 g of the title compound, mp 203-205° C.

### EXAMPLE 13

b) [(S)-1-(9-Fluoro-5-methyl-1,3-dioxo-2,3,6,7-tetrahydro-1H, 5H-pyrido[3,2,1-ij]quinazolin-8-yl)pyrrolidin-3-yl]carbamic Acid Tert-Butyl Ester

Reaction of 8,9-difluoro-5-methyl-6,7-dihydro-5H-pyrido[3,2,1-ij]quinazoline-1,3-dione (0.125 g, 0.5 mmol, [PCT Int. Appl. WO 0153273 A1]) with (S)-3-pyrrolidinylcarbamic acid tert-butyl ester [J. Med. Chem. 1992, 35, 1764] (0.37 g, 2.0 mmol) as described in Example 12a (except that reaction was carried out at 110° C. for 18 hours) gave the title compound (0.11 g).

b) 8-((S)-3-Aminopyrrolidin-1-yl)-9-fluoro-5-methyl-6,7-dihydropyrido[3,2,1-ij]quinazoline-1,3-dione Hydrochloride

A solution of 0.11 g (0.26 mmol) of [(S)-1-(9-fluoro-5-methyl-1,3-dioxo-2,3,6,7-tetrahydro-1H, 5H-pyrido[3,2,]-ij]quinazolin-8-yl)pyrrolidin-3-yl]carbamic acid tert-butyl ester (Example 13a) in 5 mL of ethanol was treated with 1 mL of ethanol saturated with hydrogen chloride gas and the reaction stirred at room temperature for 18 hours. The solvent was removed in vacuo and the residue dissolved in water, filtered through a fiber glass pad to clarify and the filtrate lyophilized to give 0.07 g of the title compound, mp 134-136° C.

### EXAMPLE 14

a) {(S)-1-[(R)-1-(9-Fluoro-5-methyl-1,3-dioxo-2,3,6,7-tetrahydro-1H, 5H-pyrido[3,2,1-ij]quinazolin-8-yl)pyrrolidin-3-yl]ethyl}carbamic Acid Tert-Butyl Ester

The reaction of 8,9-difluoro-5-methyl-6,7-dihydro-5H-pyrido[3,2,1-ij]quinazoline-1,3-dione (0.125 g, 0.5 mmol, [PCT Int. Appl. WO 0153273 A1]) with ((R)-(S)-1-pyrrolidin-3-ylethyl)carbamic acid tert-butyl ester (0.22 g, 1.0 mmol, [J. Het. Chem. 1992, 29, 1481]) and 1,1,3,3-tetramethylguanidine (0.125 mL, 1.0 mmol) in dimethyl sulfoxide (3 mL) was heated at 110° C. for 18 hours. The reaction was cooled to room temperature, diluted to 50 mL with ice and water and extracted with ethyl acetate (2×30 mL). The combined organics were washed with water (2×25 mL), dried with magnesium sulfate, filtered and concentrated in vacuo. The residue was chromatographed over flash grade silica gel (230-400 mesh) eluting with dichloromethane/ethanol (90:10) to provide the title compound (0.11 g); mp 123-125° C.

b) 8-[(R)-3-((S)-1-Aminoethyl)pyrrolidin-1-yl)-9-fluoro-5-methyl-6,7-dihydro-5H-pyrido[3,2,1-ij]quinazoline-1,3-dione Hydrochloride

A solution of 0.11 g (0.25 mmol) {(S)-1-[(R)-1-(9-fluoro-5-methyl-1,3-dioxo-2,3,6,7-tetrahydro-1H, 5H-pyrido[3,2,1-ij]quinazolin-8-yl)pyrrolidin-3-yl]ethyl}carbamic acid, tert-butyl ester (Example 14a) in 5 mL of ethanol was treated with 2 mL of ethanol saturated with hydrogen chloride gas. A precipitate formed and the mixture was then stirred at room temperature for 18 hours. The solvent was removed in vacuo and the residue triturated with ethyl ether. The resulting solid was removed by filtration, washed with ether and dried in vacuo to give 0.065 g of the title compound, mp 276-278° C.

### EXAMPLE 15

a) 4-(1-tert-Butoxycarbonyl-1-cyanomethyl)-2,5-difluoro-3-methylbenzoic Acid Ethyl Ester

A solution of 2,4,5-trifluoro-3-methylbenzoic acid ethyl ester (20 g, 92 mmol, [PCT Int. Appl. WO 0153273 A1]), potassium carbonate (30.4 g, 220 mmol) and tert-butylcyanoacetate (15.5 g, 110 mmol) in dimethyl sulfoxide (120 mL) was heated for 2 hours at 65-70° C. The resulting mixture was poured into a stirred mixture of ice water and ethyl acetate (2:1) and acidified to pH 3 with aqueous 6N hydrochloric acid. The aqueous layer was separated and the organic layer washed with water and brine. The organic extract was then dried over sodium sulfate and concentrated to afford the title compound (31 g): ¹H NMR (200 MHz, CDCl₃): δ 7.63-7.50 (m, 1H), 5.15 (s, 1H), 4.41 (q, 2H), 2.38 (d, 3H), 1.50 (s, 9H), 1.41 (t, 3H).

b) 4-Cyanomethyl-2,5-difluoro-3-methylbenzoic Acid Ethyl Ester

4-(1-tert-Butoxycarbonyl-1-cyanomethyl)-2,5-difluoro-3-methylbenzoic acid ethyl ester (10 g, 29.5 mmol, Example 15a) and a catalytic amount of p-toluenesulfonic acid (200 mg) in toluene (60 mL) was refluxed for 6 hours, then poured into ice water. The organic layer was separated, washed with water, brine and dried over sodium sulfate and concentrated. The resulting residue was purified by flash silica gel chromatography (1:5 ethyl acetate/hexanes) to afford the title compound (5.3 g): ¹H NMR (200 MHz, CDCl₃): δ 7.59-7.45 (m, 1H), 4.40 (q, 2H), 3.78 (s, 2H), 2.39 (d, 3H), 1.41 (t, 3H).

c) 4-(1-Cyanocyclopropyl)-2,5-difluoro-3-methylbenzoic Acid

Benzyltriethylammonium chloride (0.937 g, 4.2 mmol) and aqueous 10 N sodium hydroxide (8.2 mL) were added to a mixture of 4-cyanomethyl-2,5-difluoro-3-methylbenzoic acid, ethyl ester (1.0 g, 4.2 mmol, Example 15b) and 1,2-dibromoethane (1.67 g, 8.9 mmol) at 10° C. The resulting mixture was stirred at room temperature for 2 hours, then acidified with aqueous 6N hydrochloric acid and extracted with ethyl acetate. The organic layer was washed with water and brine, dried over sodium sulfate and concentrated to afford the title compound (1 g), which was used without further purification.

d) 1-{1-[4-(1-Cyanocyclopropyl)-2,5-difluoro-3-methylphenyl]methanoyl}-3-cyclopropyl Urea

A solution of 4-(1-cyanocyclopropyl)-2,5-difluoro-3-methylbenzoic acid (1 g, 4.21 mmol, Example 15c) in dichloromethane (15 mL) at 0° C. was treated with oxalyl chloride (5 mL) followed by N,N-dimethylformamide (3 drops). The mixture was stirred at room temperature for 1 hour then concentrated in vacuo. The residue was dissolved in benzene (15 mL), treated with cyclopropyl urea (0.421 g, 4.21 mmol) in benzene (10 mL), and refluxed overnight. The resulting mixture was concentrated and diluted with ethyl acetate. The solution was washed with water and brine, dried over sodium sulfate and concentrated. The resulting residue was purified by flash silica gel chromatography (1:1 ethyl acetate/hexanes) to afford the title compound (0.94 g): ¹H NMR (200 MHz, CDCl₃): δ 9.01 (bd, 1H), 8.50 (bs, 1H), 7.62-7.46 (m, 1H), 2.87-2.69 (m, 1H), 2.51 (d, 3H), 1.96-1.81 (m, 2H), 1.45-1.31 (m, 2H), 0.91-0.78 (m, 2H), 0.71-0.56 (m, 2H).

e) 1-(1-Cyclopropyl-6-fluoro-8-methyldioxo-1,2,3,4-tetrahydroquinazolin-7-yl)cyclopropanecarbonitrile

A solution of 1-1{-[4-(1-cyanocyclopropyl)-2,5-difluoro-3-methylphenyl]methanoyl}-3-cyclopropyl urea (0.640 g, 2 mmol, Example 15d) in tetrahydrofuran (20 mL) and N,N-dimethylformamide (1 mL) at −20° C. was treated with sodium hydride (60% dispersion in mineral oil, 0.280 g, 7 mmol). The resulting mixture was stirred at room temperature for 30 minutes, then refluxed for 3 days. The mixture was then diluted with ethyl acetate, washed with brine, dried over sodium sulfate and concentrated. The resulting residue was purified by column chromatography (3:2 ethyl acetate/hexanes) to afford the title compound (0.55 g): ¹H NMR (200 MHz, CDCl₃): δ 9.47 (bs, 1H), 7.70 (d, 1H), 3.50-3.33 (m, 1H), 2.79 (s, 3H), 2.00-1.82 (m, 2H), 1.48-1.33 (m, 2H), 1.30-1.08 (m, 2H), 0.69-0.51 (m, 2H).

f) 1-(1-Cyclopropyl-6-fluoro-8-methyldioxo-1,2,3,4-tetrahydroquinazolin-7-yl)cyclopropanecarboxylic Acid Amide

A solution of 1-(1-cyclopropyl-6-fluoro-8-methyldioxo-1,2,3,4-tetrahydroquinazolin-7-yl)cyclopropanecarbonitrile (0.300 g, 1 mmol, Example 15e) in aqueous 1N sodium hydroxide (2 mL) was treated with hydrogen peroxide (27% w/w, 0.252 g, 2 mmol) over 5 minutes at room temperature. The resulting mixture was stirred for 30 minutes and acidified to pH 2 with aqueous 6N hydrochloric acid. The precipitate was collected by filtration to afford the title compound (0.204 g): ¹H NMR (200 MHz, DMSO-d₆): δ 7.44 (d, 1H), 7.05 (bs, 1H), 6.63 (bs, 1H), 3.48-3.24 (m, 2H), 2.52 (s, 3H), 1.71-1.50 (m, 2H), 1.20-0.85 (m, 4H), 0.76-0.58 (m, 1H), 0.50-0.31 (m, 1H).

### EXAMPLE 16

a) 8,9-Difluoro-3-methyl-7-nitro-2,3-dihydro-1-oxa-3a,5-diazaphenalene-4,6-dione

A solution of 0.54 g (2.0 mmol) of 5-amino-8,9-difluoro-3-methyl-2,3-dihydro-1-oxa-3a,5-diazaphenalene-4,6-dione (WO 0153273) in 5 mL of 98% sulfuric acid was treated portionwise with 0.30 g (3.0 mmol) of potassium nitrate at room temperature. After the addition was complete, the reaction was stirred overnight. The solution was then poured onto 50 g of ice and water and stirred. The resulting coarse precipitate was removed by filtration, washed with water and dried in vacuo to give 0.50 g of material that was chromatographed over silica gel eluting with dichloromethane/ethanol (90/10) to give 0.23 g of the title compound, mp 286-288° C.

b) 7-Amino-8,9-difluoro-3-methyl-2,3-dihydro-1-oxa-3a,5-diazaphenalene-4,6-dione

A solution of 0.48 g (1.6 mmol) of 8,9-difluoro-3-methyl-7-nitro-2,3-dihydro-1-oxa-3a,5-diazaphenalene-4,6-dione (Example 16a) in a mixture of 50 mL of tetrahydrofuran/methanol (45-5) was treated with 0. 1 g of Raney-nickel and shaken in a hydrogen atmosphere at 22° C. and pressures of 21-49 psi for 22 hours. The catalyst was removed by filtration and the solvent removed in vacuo to give 0.42 g of the title compound, mp>280° C.

c) {(S)-1-[(R)-1-(7-Amino-8-fluoro-3-methyl-4,6-dioxo-2,3,5,6-tetrahydro-4H-1-oxa-3a,5-diazaphenalen-9-yl)pyrrolidin-3-yl]ethyl}carbamic Acid Tert-Butyl Ester

A solution of 0.20 g (0.75 mmol) of 7-amino-8,9-difluoro-3-methyl-2,3-dihydro-1-oxa-3a,5-diazaphenalene-4,6-dione (Example 16b), 0.32 g (1.5 mmol) of (R)-(S)-1-pyrrolidin-3-ylethyl)carbamic acid tert-butyl ester [J. Heterocycl. Chem. 1992, 29(6), 1481], 0.3 g (3.0 mmol) of triethylamine and 10 mL of acetonitrile was heated at reflux for 12 hours. The solvent was removed in vacuo and the residue partitioned between dichloromethane/water (75 mL each). The organic layer was then washed with water, dried with magnesium sulfate, filtered and concentrated to give 0.33 g of material that was chromatographed over silica gel eluting with dichloromethane/ethanol (97:3) to give 0.23 g of the title compound, mp 136-138° C.

d) 7-Amino-9-[9-(R)-3-((S)-1-aminoethyl)pyrrolidin-1-yl)-8-fluoro-3-methyl-2,3-dihydro-1-oxa-3a,5-diazaphenalene-4,6-dione Hydrochloride

A solution of 0.23 g (0.5 mmol) of {(S)-1-[(R)-1-(7-amino-8-fluoro-3-methyl-4,6-dioxo-2,3,5,6-tetrahydro-4H-1-oxa-3a,5-diazaphenalen-9-yl)pyrrolidin-3-yl]ethyl}carbamic acid tert-butyl ester (Example 16c) in 2 mL of ethanol was treated with 1 mL of ethanol saturated with hydrogen chloride gas and the mixture stirred at room temperature for 18 hours. The solvent was then removed in vacuo and the residue triturated with ethanol/ether (5 mL of a 1:1 solution). The solid was removed by filtration, washed with ethanol/ether (1:1) and dried in vacuo to give 0.21 g of the title compound, mp 223-225° C.

### EXAMPLE 17

a) [(3aR,6aS)- and (3aS,6aR)-2-(1-Cyclopropyl-6-fluoro-8-methoxydioxo-1,2,3,4-tetrahydroquinazolin-7-yl)octahydrocyclopenta[c]pyrrol-4-yl]carbamic Acid Tert-Butyl Ester

A solution of 0.27 g (1.0 mmol) of 1-cyclopropyl-6,7-difluoro-8-methoxy-1H-quinazolinedione, 0.45 g (2.0 mmol) of ((3aS, 6aR)- and (3aR,6aS)-2-benzyloctahydrocyclopenta[c]pyrrol-4-yl)carbamic acid tert-butyl ester [U.S. Pat. No. 5,580,872], 0.39 g (3.0 mmol) of diisopropylethylamine (Hunig's base) and 1.25 mL of dimethyl sulfoxide was heated at 90° C. for 18 hours. The reaction mixture was diluted to 15 mL with water and extracted with ethyl acetate (2×20 mL). The combined organic extracts were washed with water (2×20 mL), dried with magnesium sulfate, filtered and concentrated in vacuo. The residue (0.75 g) was chromatographed over silica gel eluting with dichloromethane/ethyl acetate (80:20) to provide 0.18 g of the title compound, mp 100-102° C.

b) 7-((3aR, 6aS)- and (3aS, 6aR)-4-Aminohexahydrocyclopenta[c]pyrrol-2-yl-1-cyclopropyl-6-fluoro-8-methoxy-1H-quinazolinedione Hydrochloride

A solution of 0.17 g (0.36 mmol) of [(3aR,6aS)- and (3aS, 6aR)-2-(1-cyclopropyl-6-fluoro-8-methoxydioxo-1,2,3,4-tetrahydroquinazolin-7-yl)octahydrocyclopenta[c]pyrrol-4-yl]carbamic acid tert-butyl ester (Example 17a) in 2 mL of ethanol was treated with 1 mL of ethanol saturated with hydrogen chloride gas and the reaction stirred at room temperature for 18 hours. The solvent was removed in vacuo and the residue dissolved in water, filtered through a fiberglass pad and lyophilized to give 0.15 g of the title compound, mp 233-235° C.

### EXAMPLE 18

a) [(3aR, 6aS)- and (3aS, 6aR)-2-(1-Cyclopropyl-6-fluoro-8-methyldioxo-1,2,3,4-tetrahydroquinazolin-7-yl)octahydrocyclopenta[c]pyrrol-4-yl]carbamic Acid Tert-Butyl Ester

A solution of 0.25 g (1.0 mmol) of 1-cyclopropyl-6,7-difluoro-8-methyl-1H-quinazolinedione, 0.45 g (2.0 mmol) of ((3aS, 6aR)- and (3aR, 6aS)-2-benzyloctahydrocyclopenta[c]pyrrol-4-yl)carbamic acid tert-butyl ester, 0.24 g (2.0 mmol) of 1,1,3,3-tetramethylguanidine and 1.5 mL of dimethyl sulfoxide was heated at 90° C. for 18 hours. The reaction mixture was diluted to 15 mL with water and the resulting solid removed by filtration, washed with water and dissolved in ethyl acetate. After drying with magnesium sulfate and filtering, the solvent was removed in vacuo and the residue chromatographed over silica gel eluting with dichloromethane/ethyl acetate (80:20) to give 0.078 g of the title compound, MSCI: m/z 459 (MH⁺).

b) 7-((3aR, 6aS)- and (3aS, 6aR)-4-Aminohexahydrocyclopenta[c]pyrrol-2-yl-1-cyclopropyl-6-fluoro-8-methyl-1H-quinazolinedione Hydrochloride

A solution of 0.078 g (0.17 mmol) of [(3aR, 6aS)- and (3aS, 6aR)-2-(1-cyclopropyl-6-fluoro-8-methyldioxo-1,2,3,4-tetrahydroquinazolin-7-yl)octahydrocyclopenta[c]pyrrol-4-yl]carbamic acid tert-butyl ester (Example 18a) in 2 mL of ethanol was treated with 1 mL of ethanol saturated with hydrogen chloride gas and the reaction stirred at room temperature for 18 hours. The solvent was removed in vacuo and the residue dissolved in water, filtered through a fiberglass pad and lyophilized to give 0.07 g of the title compound, mp 208-210 ° C.

### EXAMPLE 19

a) 1-Benzyl-3-(2-bromoacetyl)pyrrolidin-2-one

To a solution of 1-benzyl-2-oxopyrrolidine-3-carboxylic acid (10 g, 45.7 mmol [U.S. Pat. No. 5,175,157] in tetrahydrofuran/dioxane (300 mL/60 mL) at −10° C. was added 4-methylmorpholine (6.5 mL, 59.4 mmol) followed by isobutyl chloroformate (7.10 mL, 54.8 mmol). After 10 minutes, a white precipitate was filtered off and washed with tetrahydrofuran. The filtrate and wash were poured into a new Erlenmyer flask and kept at 0° C. To this mixture was added a solution of diazomethane (1.1M in ether, 55 mL). After 15 minutes, a]:I hydrobromic acid (48%)/acetic acid solution was added dropwise until gas evolution ceased. After 15 minutes, the reaction mixture was diluted with ethyl acetate and washed with saturated aqueous sodium bicarbonate. The combined organic layers were dried over magnesium sulfate, filtered and concentrated. The residue mixture was purified by chromatography (99:1 dichloromethane/methanol) to give the title compound (26.6 g); MSCI: m/z 296,298 (MH⁺).

b) 1-Benzyl-3-(2-fluoroacetyl)pyrrolidin-2-one

To a solution of 1-benzyl-3-(2-bromoacetyl)pyrrolidin-2-one (2.2 g, 7.43 mmol, Example 19a) in acetonitrile (25 mL) was added 18-crown-6 (0.980 g, 3.72 mmol) and potassium fluoride (spray dried) (2.16 g, 37.2 mmol). The reaction mixture was immersed in an oil bath at 80° C. After 1 hour, the mixture was cooled to room temperature and partitioned between water and ethyl acetate. The organic layer was dried over magnesium sulfate, filtered and concentrated. The residue was purified by chromatography (99:1 dichloromethane/methanol then 98:2 dichloromethane/methanol) to afford the title compound (0.515 g): ¹H NMR (400 MHz, CDCl₃) δ 7.36-7.19 (m, 5H), 4.93 (m, 2H), 4.46 (m, 2H), 3.61 (m, 1H), 3.48 (m, 2H), 2.74 (m, 2H).

c) 1-Benzyl-3-(1-benzylamino-2-fluoroethyl)pyrrolidin-2-one

Benzylamine (3.90 mL, 35.7 mmol) was added to a solution of 1-benzyl-3-(2-fluoroacetyl)pyrrolidin-2-one (7.00 g, 29.8 mmol, Example 19b) in 1,2-dichloroethane (150 mL). The solution was cooled to ⁰° C. and sodium triacetoxyborohydride (8.20 g, 38.7 mmol) was added. The reaction mixture was warmed to room temperature and stirred overnight, then washed with aqueous sodium bicarbonate solution and brine. The organic layer was dried over magnesium sulfate, filtered and concentrated. The residue was purified by chromatography (99:1 to 97:3 dichloromethane/methanol) to afford the title compound (7.2 g) as a mixture of diastereomers: MSCI: m/z 327 (MH⁺).

d) Benzyl-[1-(1-benzylpyrrolidin-3-yl)-2-fluoroethyl]amine

To a solution of 1-benzyl-3-(1-benzylamino-2-fluoroethyl)pyrrolidin-2-one (7.2 g, 22 mmol, Example 19c) in tetrahydrofuran (100 mL) at 0° C. was added lithium aluminum hydride (IM in tetrahydrofuran, 22 mL) dropwise. After 1 hour, the mixture was warmed to room temperature and quenched after an additional 30 minutes by the addition of 0.84 mL water, 0.84 mL 15% sodium hydroxide solution and 2.5 mL water. The reaction mixture was filtered and concentrated. The crude residue was purified by chromatography (97:3 to 90:10 dichloromethane/methanol) to give the title compound (2.5 g) as a mixture of diastereomers. MSCI: m/z=313 (MH⁺).

e) 2-Fluoro-1-pyrrolidin-3-ylethylamine

To a solution of benzyl-[1-(1-benzylpyrrolidin-3-yl)-2-fluoroethyl]amine (2.5 g, 8.0 mmol, Example 19d) in methanol (50 mL) was added 20% palladium on carbon (200 mg). Hydrogen gas was introduced to the reaction mixture at high pressure (48 psi) for 24 hours, at which time sulfuric acid (3 drops) was added. After hydrogenation for an additional 24 hours, the reaction mixture was filtered through Celite, washed with methanol, and the combined filtrate concentrated under vacuum to afford the title compound (1.0 g): MSCI: m/z 133 (MH⁺).

f) 7-[3-(1-Amino-2-fluoroethyl)pyrrolidin-1-yl]-1-cyclopropyl-6-fluoro-8-methyl-1H-quinazolinedione Hydrochloride

1-Cyclopropyl-6,7-difluoro-8-methyl-1H-quinazolinedione (0.97 g, 3.83 mmol), 2-fluoro-1-pyrrolidine-3-ylethylamine (0.66 g, 4.98 mmol, Example 19e), and 1,1,3,3-tetramethylguanidine (0.96 mL, 7.66 mmol) in dimethyl sulfoxide (1 mL) was heated to 90° C. for 16 hours. The solution was diluted with ethyl acetate and washed with saturated sodium bicarbonate, water and brine. The organic layer was dried over magnesium sulfate, filtered, and concentrated. The resulting residue was then purified by flash silica gel chromatography (90:10 dichloromethane/methanol) to afford a yellow residue. The residue was dissolved in dichloromethane (2 mL) and gaseous hydrogen chloride solution (1 mL, 2.0 M in ether). The resulting precipitate was filtered to afford the title compound (0.092 g); mp 218-221° C., MSCI: m/z 365 (MH⁺).

### EXAMPLE 20

a) 3-(Methoxymethylcarbamoyl)pyrrolidine-1-carboxylic Acid Benzyl Ester

To a solution of pyrrolidine-1,3-dicarboxylic acid benzyl ester (5.19 g, 20.8 mmol, [WO 9706802]) in dichloromethane (100 mL) was added triethylamine (4.35 mL, 31.2 mmol), N,O-dimethylhydroxylamine hydrochloride (2.44 g, 25.0 mmol), and N-(3-dimethylaminopropyl)-N′-ethylcarbodiimide hydrochloride (4.79 g, 25.0 mmol). After 5 hours, the reaction mixture was washed with saturated sodium bicarbonate, water, and brine. The organic layer was then dried over magnesium sulfate, filtered, and the filtrate concentrated. The resulting residue was purified by flash silica gel chromatography (hexanes/ethyl acetate) to give the title compound (4.30 g) as a yellow solid: MSCI: m/z 293 (MH⁺).

b) 3-Cyclopropanecarbonylpyrrolidine-1-carboxylic Acid Benzyl Ester

To a solution of cyclopropylbromide (1.76 mL, 22.0 mmol) in tetrahydrofuran (50 mL) under nitrogen atmosphere at −78° C. was added a 1.6 M solution of n-butyllithium (I 6.5 mL, 26.4 mmol) in hexanes slowly over 15 minutes. After 1 hour, 3-(methoxymethyl-carbamoyl)pyrrolidine-1-carboxylic acid benzyl ester (4.29 g, 14.7 mmol, Example 20a) was added as a solution in tetrahydrofuran (25 mL). After 30 minutes, the reaction mixture was warmed to room temperature for 1 hour. The reaction mixture was diluted with ethyl acetate, washed with saturated ammonium chloride, water, and brine. The organic layer was dried over magnesium sulfate, filtered, and the filtrate concentrated. The resulting residue was purified by flash silica gel chromatography (hexanes/ethyl acetate) to give the title compound (1.91 g) as a yellow oil: MSCI: m/z 274 (MH⁺).

c) 3-(Cyclopropylhydroxyiminomethyl)pyrrolidine-1-carboxylic Acid Benzyl Ester

To a solution of 3-cyclopropanecarbonylpyrrolidine-1-carboxylic acid benzyl ester (1.91 g, 6.99 mmol, Example 20b)in pyridine (10 mL) was added hydroxylamine hydrochloride (0.58 g, 8.4 mmol). The reaction was heated to 90° C. for 6 hours, and diluted with ethyl acetate. The organic layer was washed twice with 1N hydrochloric acid, water, and brine. The organic layer was dried over magnesium sulfate, filtered, and the filtrate concentrated. The resulting residue was purified by flash silica gel chromatography (ethyl acetate) to give the title compound (1.60 g) as a brown oil: MSCI: m/z 289 (MH⁺).

d) C-Cyclopropyl-C-pyrrolidin-3-ylmethylamine

To a solution of 3-(cyclopropylhydroxyiminomethyl)pyrrolidine-1-carboxylic acid benzyl ester (1.60 g, 5.53 mmol, Example 20c) in methanol (50 mL) was added Raney nickel (1 g). Hydrogen was introduced to the reaction mixture at high pressure (47 psi) for 72 hours, then the reaction mixture was filtered through Celite, washed with methanol, and the combined filtrates concentrated under vacuum to afford the title compound (1.32 g) as a brown oil: MSCI: m/z 141 (MH⁺).

e) 7-[3-(Aminocyclopropylmethyl)pyrrolidin-1-yl]-1-cyclopropyl-6-fluoro-8-methyl-1H-quinazolinedione Hydrochloride

Utilizing the same procedure as described in Example 19f, from reaction of 1-cyclopropyl-6,7-difluoro-8-methyl-1H-quinazolinedione (0.83 g, 3.30 mmol), C-cyclopropyl-C-pyrrolidin-3-ylmethylamine (0.69 g, 4.95 mmol, Example 20d), and 1,1,3,3-tetramethylguanidine (1.24 mL, 9.90 mmol) to afford the title compound (0.040 g); mp 191-193° C.; MSCI: m/z 373 (MH⁺).

### EXAMPLE 21

a) 4-Hydroxymethyl-1-((S)-1-phenylethyl)pyrrolidin-2-one

To a solution of 5-oxo-1-(1-phenylethyl)pyrrolidine-3-carboxylic acid methyl ester (11.17 g, 45.17 mmol, [J. Het. Chem.1992, 29, 1481]) in tetrahydrofuran (100 mL) was added lithium chloride (3.83 g, 90.34 mmol), sodium borohydride (3.42 g, 90.34 mmol), and ethanol (200 mL). After 20 hours, saturated ammonium chloride (50 mL) was added and the reaction mixture concentrated under vacuum. The resulting residue was dissolved in ethyl acetate and washed with saturated ammonium chloride, water, and brine. The organic layer was dried over magnesium sulfate, filtered, and the filtrate concentrated to give the title compound (8.61 g): MSCI: m/z 220 (MH⁺).

b) Methanesulfonic acid 5-oxo-1-((S)-1-phenylethyl)pyrrolidin-3-ylmethyl Ester

To a solution of 4-hydroxymethyl-1-((S)-1-phenylethyl)pyrrolidin-2-one (5.35 g, 24.39 mmol, Example 21a) in dichloromethane (25 mL) at 0° C. was added triethylamine (4.42 mL, 31.7 mmol) and methanesulfonyl chloride (1.93 mL, 25.0 mmol). After 15 minutes the reaction mixture was warmed to room temperature for 4 hours. The reaction mixture was diluted with dichloromethane and washed with saturated sodium bicarbonate, water, and brine. The organic layer was then dried over magnesium sulfate, filtered, and the filtrate concentrated. The resulting residue was purified by flash silica gel column chromatography (1:1 hexanes/ethyl acetate to ethyl acetate gradient) to give the title compound (7.31 g) as a yellow oil: MSCI: m/z 298 (MH⁺).

c) 4-Fluoromethyl-1-((S)-1-phenylethyl)pyrrolidin-2-one

To a solution of methanesulfonic acid 5-oxo-1-(1-phenylethyl)pyrrolidin-3-ylmethyl ester (7.30 g, 24.5 mmol, Example 21b) in tetrahydrofuran (100 mL) was added tetrabutylammonium fluoride hydrate (9.64 g, 36.9 mmol). The reaction mixture was refluxed for 12 hours, cooled to room temperature, and concentrated. The resulting residue was purified by flash silica gel chromatography (ethyl acetate) to give the title compound (4.02 g) as a yellow oil: MSCI: m/z 222 (MH⁺).

d) (3S, 4R)- and (3R, 4S)-3-Benzyloxymethyl-4-fluoromethyl-1-((S)-1-phenylethyl)pyrrolidin-2-one

To a solution of 4-fluoromethyl-1-(1-phenylethyl)pyrrolidin-2-one (4.0 g, 18.17 mmol, Example 21 c) in tetrahydrofuran (100 mL) under nitrogen atmosphere at −78° C. was added lithium diisopropylamide (2 M in heptane/tetrahydrofuran/ethylbenzene, 10.9 mL, 21.8 mmol) slowly over 15 min. After 1 hour, benzyl chloromethyl ether (3.03 mL, 21.80 mmol) was added. After 30 minutes, the reaction mixture was warmed to room temperature for 1 hour. The reaction mixture was diluted with ethyl acetate, washed with saturated ammonium chloride, water, and brine. The organic layer was then dried over magnesium sulfate, filtered, and the filtrate concentrated. The resulting residue was purified by flash silica gel chromatography (hexanes/ethyl acetate) to provided the title compound (1.91 g) as a yellow oil: MSCI: m/z 342 (MH⁺).

e) (3S, 4R)- and (3R, 4S)-4-Fluoromethyl-3-hydroxymethyl-1-((S)-1-phenylethyl)pyrrolidin-2-one

To a solution of (3S, 4R)- and (3R, 4S)-3-benzyloxymethyl-4-fluoromethyl-1-((S)-1-phenylethyl)pyrrolidin-2-one (4.95 g, 14.5 mmol, Example 21d) in methanol (50 mL) was added 20% palladium on carbon (0.5 g). Hydrogen gas was introduced to the reaction mixture at high pressure (48 psi) for 2 hours, then the reaction mixture was filtered through diatomaceous earth, washed with methanol, and the combined filtrate concentrated under vacuum to afford the title compound (3.50 g) as an oil: MSCI: m/z 252 (MH⁺).

f) [(3R, 4R)- and (3S, 4S)-4-Fluoromethyl-1-((S)-1-phenylethyl)pyrrolidin-3-yl]methanol

To a solution of (3S, 4R)- and (3R, 4S)-4-fluoromethyl-3-hydroxymethyl-1-((S)-1-phenylethyl)pyrrolidin-2-one (3.50 g, 13.93 mmol, Example 21 e) in tetrahydrofuran (50 mL) was added lithium aluminum hydride (IM in tetrahydrofuran, 28 mL, 28 mmol) slowly over 10 min. The reaction mixture was refluxed for 3 hours and cooled to room temperature. While stirring, water (1 mL), 15% sodium hydroxide solution (1 mL), and water (3 mL) were added. After 15 min, magnesium sulfate was added and the mixture was filtered. The solid was washed with tetrahydrofuran and the combined filtrate concentrated under vacuum to give the title compound (3.35 g) as a clear oil: MSCI: m/z 238 (MH⁺).

g) (3R, 4R)- and (3S, 4S)-4-Fluoromethyl-1-((S)-1-phenylethyl)pyrrolidin-3-yl-methanesulfonic Acid Methyl Ester

To a solution of [(3R, 4R)- and (3S, 4S)-4-fluoromethyl-1-((S)-1-phenylethyl)pyrrolidin-3-yl]methanol (3.35 g, 14.12 mmol, Example 21f) in dichloromethane (25 mL) at 0° C. was added triethylamine (2.95 mL, 21.18 mmol) and methanesulfonyl chloride (1.20 mL, 15.53 mmol). After 15 minutes the reaction mixture was warmed to room temperature for 4 hours. The reaction mixture was then diluted with dichloromethane and washed with saturated sodium bicarbonate, water, and brine. The organic layer was subsequently dried over magnesium sulfate, filtered, and the filtrate concentrated. The resulting residue was purified by flash silica gel chromatography (1:1 hexanes/ethyl acetate to ethyl acetate gradient) to give the title compound (3.41 g) as a yellow oil; MSCI: m/z 316 (MH⁺).

h) (3R, 4R)- and (3S, 4S)-3-Azidomethyl-4-fluoromethyl-1-((S)-1-phenylethyl)pyrrolidine

To a solution of (3R, 4R)- and (3S, 4S)-4-fluoromethyl-1-((S)-1-phenylethyl)pyrrolidin-3-yl-methanesulfonic acid methyl ester (3.41 g, 10.8 mmol, Example 21g) in N,N-dimethylformamide (10 mL) was added sodium azide (2.81 g, 43.3 mmol). The reaction mixture was heated to 90° C. for 16 hours, cooled to room temperature, and diluted with ethyl acetate. The organic mixture was washed twice with water and brine. The organic layer was dried over magnesium sulfate, filtered, and concentrated. The resulting residue was purified by flash silica gel chromatography (1:1 hexanes/ethyl acetate to ethyl acetate gradient) to give the title compound (1.93 g) as a yellow oil; MSCI: m/z 263 (MH⁺).

i) (3R, 4R)- and (3S, 4S)-3-Azidomethyl-4-fluoromethylpyrrolidine-1-carboxylic Acid Benzyl Ester

To a solution of (3R, 4R)- and (3S, 4S)-3-azidomethyl-4-fluoromethyl-1-((S)-1-phenylethyl)pyrrolidine (1.93 g, 7.36 mmol, Example 21h) in 1,2-dichloroethane (50 mL) was added benzyl chloroformate (1.57 mL, 11.04 mmol). The reaction mixture was refluxed for 4 hours, cooled to room temperature, and concentrated under vacuum. The resulting residue was then purified by flash silica gel chromatography (1:1 hexanes/ethyl acetate to ethyl acetate gradient) to give the title compound (2.58 g) as a oil: MSCI: m/z 293 (MH⁺).

j) C-((3S, 4R)- and (3R, 4S)-4-Fluoromethylpyrrolidin-3-yl)methylamine

To a solution of (3R, 4R)- and (3S, 4S)-3-azidomethyl-4-fluoromethylpyrrolidine-1-carboxylic acid benzyl ester (2.58 g, 8.84 mmol, Example 21i) in tetrahydrofuran (100 mL) was added 10% Pd/C (0.39 g). Hydrogen was introduced to the reaction mixture at high pressure (48 psi) for 5 days, then the reaction mixture was filtered through Celite, washed with methanol, and the combined filtrate concentrated under vacuum to afford the title compound (1.36 g) as an oil: MSCI: m/z 133 (MH⁺).

k) 7-((3S, 4R)- and (3R, 4S)-3-Aminomethyl-4-fluoromethylpyrrolidin-1-yl)-1-cyclopropyl-6-fluoro-8-methyl-1H-quinazolinedione

Utilizing the same procedure as described in Example]9f, from reaction of 1-cyclopropyl-6,7-difluoro-8-methyl-1H-quinazolinedione (0.26 g, 1.03 mmol), C-((3S, 4R)- and (3R, 4S)-4-fluoromethylpyrrolidin-3-yl)methylamine (0.27 g, 2.06 mmol, Example 21i), and 1,1,3,3-tetramethylguanidine (0.25 mL, 2.06 mmol) afforded the title compound (0.0073 g); mp 175-178° C.: MSCI: m/z 365 (MH⁺).

### EXAMPLE 22

a) 4-Bromo-2,5-difluoro-3-methylbenzoic Acid

An ice-chilled solution of acetonitrile (40 mL) and tert-butyl nitrite (5.9 mL, 49.6 mmol) was treated with copper(II) bromide (8.9 g, 39.8 mmol) and allowed to stir. After 15 minutes, a solution of 4-amino-2,5-difluoro-3-methylbenzoic acid [PCT Int. Appl. Ser. No. WO 96/05192 A1](6.2 g, 33.1 mmol) in acetonitrile (300 mL) was added by addition funnel, and the reaction mixture warmed to room temperature and stirred for 19 hours. The mixture was concentrated in vacuo, and the resulting residue dissolved in ethyl acetate and washed with 1 N hydrochloric acid, water and brine. The combined organic layers were dried over magnesium sulfate, filtered, and concentrated in vacuo to afford the title compound (6.8 g): ¹H NMR (400 MHz, DMSO-d₆) δ 13.62 (bs, 1H), 7.61 (dd, 1H), 2.33 (d, 3H).

b) 4-Bromo-2,5-difluoro-3-methylbenzamide

To a solution of 4-bromo-2,5-difluoro-3-methylbenzoic acid (6.8 g, 27 mmol, Example 22a) in dichloromethane (90 ml) was added oxalyl chloride (3.5 mL, 40 mmol) and 10 drops of N,N-dimethylformamide. The reaction mixture was stirred for 15 hours at room temperature and concentrated in vacuo. The resulting residue was dissolved in dichloromethane (100 mL) and concentrated in vacuo. The residue was re-dissolved in dichloromethane, cooled to 0° C. and ammonia gas bubbled through the solution for 15 minutes. The mixture was allowed to warm to room temperature and stirred for 1 hour. The mixture was partitioned between aqueous saturated sodium bicarbonate and ethyl acetate. The aqueous phase was extracted with ethyl acetate. The combined organic layers were dried over magnesium sulfate, filtered, and concentrated in vacuo to afford the title compound (6.8 g): MSCI: m/z 250 (MH⁺); ¹H NMR (400 MHz, CDCl₃) δ 7.82 (bs, 1H), 7.77 (bs, 1H), 7.44 (dd, 1H), 2.33 (d, 3H).

c) 1-(4-Bromo-2,5-difluoro-3-methylbenzoyl)-3-cyclopropylurea

To a solution of 4-bromo-2,5-difluoro-3-methylbenzamide (6.8 g, 27.2 mmol, Example 22b) in 1, 2-dichloroethane (60 mL) was added oxalyl chloride (4.7 mL, 53.9 mmol) and the resulting mixture heated at 90° C. for 2 hours. The reaction mixture was then cooled to room temperature, and concentrated in vacuo. The resulting residue was dissolved in dichloromethane (50 mL), concentrated in vacuo and re-dissolved in dichloromethane (50 mL), cooled to 0° C. and treated with cyclopropylamine (2.8 mL, 40.4 mmol). The reaction mixture was warmed to room temperature for 1 hour and partitioned between ethyl acetate and saturated sodium bicarbonate. The aqueous phase was extracted with ethyl acetate. The combined organics layers were dried over magnesium sulfate, filtered, and concentrated in vacuo to obtain the title compound (8.0 g): ¹H NMR (400 MHz, CDCl₃) δ 8.59 (bs, 1H), 8.46 (bs, 1H), 7.60 (m, 1H), 2.76 (m, 1H), 2.41 (d, 3H), 0.81 (m, 2H), 0.62 (m, 2H).

d) 7-Bromo-1-cyclopropyl-6-fluoro-8-methyl-1H-quinazolinedione

To a solution of 1-(4-bromo-2,5-difluoro-3-methylbenzoyl)-3-cyclopropylurea (3.8 g, 11.4 mmol, Example 22c) in tetrahydrofuran (40 mL) at 0 ° C. was added potassium bis(trimethylsilyl)amide (57 mL, 28.5 mmol, 0.5 M in toluene) over 15 minutes. The reaction mixture was warmed to room temperature, and 18-crown-6 (1.30 g, 4.92 mmol) added. The mixture was heated at 80° C. for 3 hours, cooled to room temperature, diluted with ethyl acetate, and washed with 1 N hydrochloric acid. The aqueous phase was extracted with ethyl acetate and the combined organic layers dried over magnesium sulfate, filtered, and concentrated in vacuo. The residue was purified by flash silica gel chromatography (hexanes to 40:60 hexanes/ethyl acetate gradient) to afford the title compound (2.0 g); MSCI: m/z 313 (MH⁺); ¹H NMR (400 MHz, CDCl₃) δ 8.42 (bs, 1H), 7.71 (d, 1H), 3.40 (m, 1H), 2.73 (s, 3H), 1.18 (m, 2H), 0.63 (m, 2H).

e) 4-Bromothiophene-2-carbaldehyde O-benzyloxime

To a solution of 4-bromothiophene-2-carboxaldehyde (10.3 g, 53.9 mmol) in ethanol (100 mL) was added O-benzylhydroxylamine hydrochloride (13.0 g, 81.4 mmol) followed by pyridine (7.0 mL). The reaction mixture was heated at 80° C. for 20 hours, cooled to room temperature and concentrated in vacuo. The resulting residue was partitioned between water and ethyl acetate. The aqueous phase was extracted with ethyl acetate (3 times), the combined organic layers washed with brine, dried over magnesium sulfate, filtered and concentrated in vacuo. Purification by flash silica gel chromatography (hexanes to 50:50 hexanes/ethyl acetate gradient) afforded the title compound (16 g) as a yellow liquid; MSCI: m/z 296 (MH⁺).

f) C-(4-Bromothiophen-2-yl)methylamine

To a solution of 4-bromothiophene-2-carbaldehyde O-benzyloxime (8.0 g, 27 mmol, Example 22e) in tetrahydrofuran (20 mL) was added borane-tetrahydrofuran complex (60 mL, 60 mmol, 1.0 M in tetrahydrofuran), and the mixture heated at 70° C. for 20 hours. The reaction mixture was cooled to room temperature and 1 N sodium hydroxide added. The mixture was extracted with ethyl acetate (3 times), the organic layers combined and washed with brine, dried over magnesium sulfate, filtered, and concentrated in vacuo to afford the title compound; MSCI: m/z 192 (MH⁺).

g) (4-Bromothiophen-2-ylmethyl)carbamic Acid Tert-Butyl Ester

To a solution of C-(4-bromothiophen-2-yl)methylamine (5.2 g, 27.1 mmol, Example 22f) in dichloromethane (100 mL) was added di-tert-butyl dicarbonate (8.8 g, 40.3 mmol) followed by triethylamine (15 mL). After 4 hours, 1 N hydrochloric acid was added and the mixture extracted with dichloromethane. The combined organic layers were washed with brine, dried over magnesium sulfate, filtered and concentrated in vacuo. Purification by flash silica gel chromatography (hexanes to 80:20 hexanes/ethyl acetate gradient) affords the title compound (3.8 g): ¹H NMR (CDCl₃) δ 7.10 (s, 1H), 6.86 (s, 1H), 4.92 (bs, 1H), 4.42 (m, 2H), 1.46 (s, 9H).

h) (4-Tributylstannylthiophen-2-ylmethyl)carbamic Acid, Tert-Butyl Ester

To a solution of (4-bromothiophen-2-ylmethyl)carbamic acid tert-butyl ester (2.1 g, 7.19 mmol, Example 22g) in diethyl ether (15 mL) at −78° C. was added methyllithium (5.1 mL, 7.14 mmol, 1.4 M in diethyl ether). After 20 minutes, n-butyllithium (14 mL, 22.4 mmol, 1.6 M in hexanes) was added, and the mixture stirred for 1 hour then treated with tributyltin chloride (7.8 mL, 28.8 mmol). After 3 hours, the mixture was warmed to room temperature and partitioned between ethyl acetate and water. The aqueous layer was extracted with ethyl acetate and the organic layers combined, washed with brine, dried over magnesium sulfate, filtered and concentrated in vacuo. Purification by flash silica gel chromatography (1:99 triethylamine/hexanes to 1:80:19 triethylamine/hexanes/ethyl acetate gradient) afforded the title compound (1.8 g): ¹H NMR (400 MHz, CDCl₃) δ 7.19 (s, 1H), 6.94 (s, 1H), 4.84 (bs, 1H), 4.51 (bs, 2H)-, 1.59-1.41 (m, 15H), 1.40-1.28 (m, 6H), 1.05-0.92 (m, 6H), 0.89 (m, 9H).

i)[4-(1-Cyclopropyl-6-fluoro-8-methyldioxo-1,2,3,4-tetrahydroquinazolin-7-yl)-thiophen-2-ylmethyl]carbamic Acid Tert-Butyl Ester

To a slurry of 7-bromo-1-cyclopropyl-6-fluoro-8-methyl-1H-quinazolinedione (0.250 g, 0.798 mmol, Example 22d) in toluene (2 mL) was added tris(dibenzylideneacetone)dipalladium(0) (0.150 g, 0.164 mmol) and triphenylarsine (0.200 g, 0.653 mmol). After 10 minutes, (4-tributylstannylthiophen-2-ylmethyl)carbamic acid tert-butyl ester (1.00 g, 1.99 mmol, Example 22h) in toluene (3 mL) was added and the mixture heated at 110° C. for 24 hours. The mixture was then cooled, diluted with ethyl acetate and poured into 10% aqueous potassium fluoride. After 1.5 hours, the mixture was filtered through diatomaceous earth. The recovered organics were washed with brine, dried over magnesium sulfate, filtered and concentrated in vacuo. The residue was triturated with hexanes and the resulting solid purified by flash silica gel chromatography (dichloromethane to 50:50 dichloromethane:ethyl acetate gradient) to afford the title compound (0.191 g); MSCI: m/z 446 (MH⁺); ¹H NMR (400 MHz, CDCl₃) δ 8.53 (bs, 1H), 7.68 (d, 1H), 7.21 (s, 1H), 6.93 (s, 11H), 4.98 (bs, 1H), 4.51 (bs, 2H), 3.36 (m, 1H), 2.43 (s, 3H), 1.46 (s, 9H), 1.18 (m, 2H), 0.80 (m, 2H).

j) 7-(5-Aminomethylthiophen-3-yl)-1-cyclopropyl-6-fluoro-8-methyl-1H-quinazoline-2,4-dione Hydrochloride

Hydrogen chloride gas was bubbled into a cooled solution (0° C.) of [4-(1-cyclopropyl-6-fluoro-8-methyldioxo-1,2,3,4-tetrahydroquinazolin-7-yl)-thiophen-2-ylmethyl]carbamic acid tert-butyl ester (0.191 g, 0.429 mmol, Example 22i) in methanol (10 mL). The reaction mixture was warmed to room temperature and stirred for 20 hours. The mixture was then concentrated in vacuo, and the resulting solid washed with hexanes and dried to afford the title compound (0.109 g); mp 205-208° C.: ¹H NMR (400 MHz, DMSO-d₆) δ 8.37 (bs, 3H), 7.71 (s, 1H), 7.53 (d, 1H), 7.30 (s, 1H), 4.28 (bs, 2H), 3.30 (m, 1H), 2.36 (s, 3H), 1.02 (m, 2H), 0.60 (m, 2H).

### EXAMPLE 23

a) 7-[4-(tert-Butyldimethylsilanyloxy)-5,5-difluoro-4,5,6,7-tetrahydrobenzo[b]thiophen-2-yl]-1-cyclopropyl-6-fluoro-8-methyl-1H-quinazolinedione

To a slurry of 7-bromo-1-cyclopropyl-6-fluoro-8-methyl-1H-quinazolinedione (1.03 g, 3.29 mmol, Example 22d) in toluene (8 mL) was added tris(dibenzylideneacetone)dipalladium(0) (0.301 g, 0.332 mmol) and triphenylarsine (0.403 g, 1.32 mmol). After 10 min, tert-butyl(5,5-difluoro-2-tributylstannyl-4,5,6,7-tetrahydrobenzo[b]thiophen-4-yloxy)dimethylsilane (3.9 g, 6.57 mmol, [WO 01/32655]) in toluene (6 mL) was added and the resulting slurry heated at 110° C. for 20 hours. The mixture was cooled, diluted with ethyl acetate, and poured into 10% aqueous potassium fluoride. After 3 hours, the mixture was filtered through diatomaceous earth. The recovered organics were washed with brine, dried over magnesium sulfate, filtered, and concentrated in vacuo. The residue was triturated with hexanes and the resulting solid purified by flash silica gel chromatography (hexanes to 50:50 hexanes/ethyl acetate gradient) to afford the title compound (1.5 g): MSCI: m/z 537 (MH⁺); ¹H NMR (400 MHz, CDCl₃) δ 8.13 (s, 1H), 7.71 (d, 1H), 6.87 (s, 1H), 4.77 (m, 1H), 3.37 (m, 1H), 3.06 (m, 2H), 2.52 (s, 3H), 2.55-2.46 (m, 1H), 2.27 (m, 1H), 1.23-1.13 (m, 2H), 0.91 (s, 9H), 0.71 (m, 2H), 0.19 (s, 3H), 0.18 (s, 3H).

b) 1-Cyclopropyl-7-(5,5-difluoro-4-hydroxy-4,5,6,7-tetrahydrobenzo[b]thiophen-2-yl)-6-fluoro-8-methyl-1H-quinazolinedione

To a cooled solution (0° C.) of 7-[4-(tert-butyldimethylsilanyloxy)-5,5-difluoro-4,5,6,7-tetrahydrobenzo[b]thiophen-2-yl]-1-cyclopropyl-6-fluoro-8-methyl-1H-quinazolinedione (0.70 g, 1.30 mmol, Example 23a) in tetrahydrofuran (13 mL) was added tetrabutylammonium fluoride (5.2 mL, 5.20 mmol, 1M in tetrahydrofuran). After 1 hour, the reaction mixture was warmed to room temperature and partitioned between ethyl acetate and saturated ammonium chloride. The combined organic extracts were washed with brine, dried over magnesium sulfate, filtered, and concentrated in vacuo. The resulting residue was purified by flash silica gel chromatography (hexanes to ethyl acetate gradient) to afford the title compound (0.48 g): MSCI: m/z 423 (MH⁺); ¹H NMR (400 MHz, CDCl₃) δ 8.27 (s, 1H), 7.70 (dd, 1H), 7.02 (s, 1H), 4.83 (m, 1H), 3.35 (m, 1H), 3.07 (m, 2H), 2.52 (s, 3H), 2.48 (m, 2H), 2.31 (m, 1H), 1.18 (m, 2H), 0.69 (m, 2H).

### EXAMPLE 24

a) 2-(1-Cyclopropyl-6-fluoro-8-methyldioxo-1,2,3,4-tetrahydro-quinazolin-7-yl)-5,5-difluoro-4,5,6,7-tetrahydrobenzo[b]thiophen-4-yl Phosphoric Acid Diphenyl Ester

To a solution of 1-cyclopropyl-7-(5,5-difluoro-4-hydroxy-4,5,6,7-tetrahydro-benzo[b]thiophen-2-yl)-6-fluoro-8-methyl-1H-quinazolinedione (0.80 g, 1.89 mmol, Example 23b) in dichloromethane (35 mL) was added diphenylphosphoryl azide (0.81 mL, 3.76 mmol) followed by 1,8-diazabicyclo[5.4.0]undec-7-ene (0.65 mL, 4.35 mmol). After 24 hours, the reaction mixture was partitioned between ethyl acetate and saturated ammonium chloride. The organics were washed with brine, dried over magnesium sulfate, filtered, and concentrated in vacuo. The resulting residue was purified by flash silica gel chromatography (hexanes to ethyl acetate gradient) affording the title compound (0.73 g); MSCI: m/z 655 (MH⁺); ¹H NMR (400 MHz, CDCl₃) δ 8.03 (s, 1H), 7.73 (d, 1H), 7.34 (m, 2H), 7.24-7.18 (m, 5H), 7.12-7.04 (m, 3H), 6.90 (s, 1H), 5.66 (m, 1H), 3.38 (m, 1H), 3.09 (m, 2H), 2.50-2.36 (m, 2H), 2.40 (s, 3H), 1.11(m, 2H), 0.63 (m, 2H).

b) 7-(4-Azido-5,5-difluoro-4,5,6,7-tetrahydrobenzo[b]thiophen-2-yl)-1-cyclopropyl-6-fluoro-8-methyl-1H-quinazolinedione

To a solution of phosphoric acid 2-(1-cyclopropyl-6-fluoro-8-methyldioxo-1,2,3,4-tetrahydroquinazolin-7-yl)-5,5-difluoro-4,5,6,7-tetrahydrobenzo[b]thiophen-4-ylphosphoric acid diphenyl ester (0.73 g, 1.12 mmol, Example 24a) in dimethyl sulfoxide (11 mL) was added sodium azide (0.72 g, 11.1 mmol) and the reaction mixture heated at 85° C. for 22 hours. The reaction mixture was cooled to room temperature, partitioned between water and ethyl acetate and extracted with ethyl acetate. The combined extracts were dried over magnesium sulfate, filtered, and concentrated in vacuo. The resulting residue was purified by flash silica gel chromatography (hexanes to 50:50 ethyl acetate/hexanes gradient) to afford the title compound (0.46 g); MSCI: m/z 448 (MH⁺); ¹H NMR (400 MHz, CDCl₃) δ 8.48 (s, 1H), 7.72 (d, 1H), 6.95 (s, 1H), 4.63 (m, 1H), 3.36 (m, 1H), 3.10 (m, 2H), 2.51 (s, 3H), 2.49-2.33 (m, 2H), 1.19 (m, 2H), 0.70 (m, 2H).

c) [2-(1-Cyclopropyl-6-fluoro-8-methyldioxo-1,2,3,4-tetrahydroquinazolin-7-yl)-5,5-difluoro-4,5,6,7-tetrahydrobenzo[b]thiophen-4-yl]carbamic Acid Tert-Butyl Ester

To a solution of 7-(4-azido-5,5-difluoro-4,5,6,7-tetrahydrobenzo[b]thiophen-2-yl)-1-cyclopropyl-6-fluoro-8-methyl-1H-quinazolinedione (0.50 g, 1.12 mmol, Example 24b) in ethanol (10 mL) and dichloromethane (3 mL) was added palladium hydroxide (0.158 g, 0.225 mmol, 20 wt. % on carbon), di-tert-butyl dicarbonate (1.2 g, 5.5 mmol), and triethylsilane (1.44 mL, 9.02 mmol). After 20 hours, the reaction mixture was filtered through diatomaceous earth. The recovered organics were washed with water, brine, dried over magnesium sulfate, filtered, and concentrated in vacuo. The resulting residue was then purified by flash silica gel chromatography (hexanes, then 50:50 hexanes:ethyl acetate gradient) to afford the title compound (0.34 g) as a white solid: MSCI: m/z 522 (MH⁺); ¹H NMR (400 MHz, CDCl₃) δ 8.51 (s, 1H), 7.69 (d, J=8.3, 1H), 6.86 (s, 1H), 5.21 (m, 1H), 5.05 (m, 1H), 3.35 (m, 1H), 3.05 (m, 2H), 2.49 (s, 3H), 2.45 (m, 2H), 1.47 (s, 9H), 1.17 (m, 2H), 0.68 (m, 2H).

d) 7-(4-Amino-5,5-difluoro-4,5,6,7-tetrahydrobenzo[b]thiophen-2-yl)-1-cyclopropyl-6-fluoro-8-methyl-1H-quinazolinedione Hydrochloride

Hydrogen chloride gas was bubbled into a cooled solution (0° C.) of [2-(1-cyclopropyl-6-fluoro-8-methyldioxo-1,2,3,4-tetrahydroquinazolin-7-yl)-5,5-difluoro-4,5,6,7-tetrahydro-benzo[b]thiophen-4-yl]carbamic acid tert-butyl ester (0.37 g, 0.713 mmol, Example 24c) in a mixture of methanol (5 mL) and dichloromethane (5 mL) for 15 minutes. The reaction mixture was then warmed to room temperature and stirred for 3 hours. The mixture was concentrated in vacuo, and the resulting solid triturated with hexanes and dried to provide the title compound (0.308 g); mp 245-250° C.: ¹H NMR (400 MHz, DMSO-d₆) δ 11.53 (s, 1H), 9.11 (bs, 2H), 7.56 (d, 1H), 7.43 (s, 1H), 5.01 (m, 1H), 3.30 (m, 1H), 3.09-3.02 (m, 2H), 2.63-2.50 (m, 2H), 2.44 (s, 3H), 1.03 (m, 2H), 0.60 (m, 2H); MSCI: m/z 422 (MH⁺).

### EXAMPLE 25

a) 2,4,5-Trifluoro-3-methylbenzoic Acid Methyl Ester

A solution of 2,4,5-trifluoro-3-methylbenzoic acid (32 g, 0.17 mol, [Japanese Appl. JP 95-219069]) in methanol (1000 mL) was cooled to ⁰° C. and saturated with hydrogen chloride gas. The resulting solution was stirred at room temperature for 1 hour, then refluxed overnight. The solvent was removed in vacuo and the residue was partitioned between ether (1000 mL) and water (200 mL). The organic layer was separated, washed with brine, dried over sodium sulfate, and concentrated. The residue was purified by column chromatography (1:9 ethyl acetate/hexanes) to afford the title compound (32.4 g): ¹H NMR (200 MHz, CDCl₃) δ 7.62 (m, 1H), 3.93 (s, 3H), 2.27 (m, 3H).

b) 4-Benzylamino-2,5-difluoro-3-methylbenzoic Acid Methyl Ester

A solution of 2,4,5-trifluoro-3-methylbenzoic acid methyl ester (26.5 g, 130 mmol, Example 25a), benzylamine (27.8 g, 260 mmol), and triethylamine (65.6 g, 650 mmol) in 250 mL of dimethylsulfoxide was heated at 100° C. for 18 hours, then cooled to room temperature. Ethyl acetate (1000 mL) and water (200 mL) were added, and the organic layer was separated, washed with brine, dried over sodium sulfate, and concentrated. The residue was purified by column chromatography (1:6 ethyl acetate/hexanes) to afford the title commpound (30.2 g): ¹H NMR (200 MHz, CDCl₃) δ 7.45 (dd, 1H), 7.32 (m, 5H), 4.59 (s, 2H), 4.05 (bs, 1H), 3.87 (s, 3H), 2.10 (d, 3H).

c) 4-Amino-2,5-difluoro-3-methylbenzoic Acid Methyl Ester

A suspension of 20% palladium on carbon (20 g), 4-benzylamino-2,5-difluoro-3-methylbenzoic acid methyl ester (24.7 g, 0.085 mol, Example 25b), ammonium formate (26.8 g, 0.425 mol) and methanol (500 mL) was heated at reflux for 4 hours. The reaction mixture was cooled to room temperature, filtered though diatomaceous earth and the solvent removed in vacuo to give a solid which was recrystallized from ethyl acetate/hexanes to afford the title compound (14.6 g): ¹H NMR (200 MHz, CDCl₃) δ 7.47 (dd, 1H), 4.23 (bs, 1H), 3.87 (s, 3H), 2.10 (d, 3H).

d) 2,5-Difluoro-4-iodo-3-methylbenzoic Acid Methyl Ester

A room temperature suspension of 4-amino-2,5-difluoro-3-methylbenzoic acid methyl ester (5.0 g, 25.0 mmol, Example 25c) and CuI (7.0 g, 37.5 mmol) in acetonitrile (250 mL) was treated dropwise with isoamyl nitrite (5.85 g, 50.0 mmol). The mixture was stirred at room temperature for 1 hour, then heated to 50° C. for 1 hour. The solvent was removed in vacuo and the residue was dissolved in ethyl acetate (500 mL), washed with 1N hydrochloric acid (50 mL) and brine (2×50 mL). After drying over sodium sulfate and concentrating in vacuo, the residue was purified by column chromatography (1:10 ethyl acetate/hexanes) to afford the title compound (7.2 g); ¹H NMR (200 MHz, CDCl₃) δ 7.47 (dd, 1H), 3.93 (s, 3H), 2.46 (d, 3H).

e) 2,5-Difluoro-4-iodo-3-methylbenzoic Acid

A solution of 2,5-difluoro-4-iodo-3-methylbenzoic acid methyl ester (3.74 g, 12.0 mmol, Example 25d) in a mixture of 2 N sodium hydroxide (50 mL) and methanol (50 mL) was heated at 60° C. for 2 hours, then cooled to room temperature. The methanol was removed in vacuo and the solution acidified with 2 N HCl to pH 3. The white precipitate was collected by filtration, washed with water and dried to give the title compound as a white solid (3.3 g). ¹H NMR (200 MHz, CDCl₃) δ 13.57 (bs, 1H), 7.50 (dd, 1H), 2.39 (d, 3H).

f) 2,5-Difluoro-4-iodo-3-methylbenzamide

A mixture of 2,5-difluoro-4-iodo-3-methylbenzoic acid (2.98 g, 10 mmol, Example 25e) and oxalyl chloride (1.52 g, 12 mmol) in 20 mL of dichloromethane was treated with 2 drops of dimethyl formamide, and stirred at room temperature for 2 hours. The mixture was concentrated in vacuo and the residue dissolved in dry tetrahydrofuran (10 mL). This solution was slowly added to a −78° C. solution of diethyl ether (40 mL) saturated with gaseous ammonia. After the addition, the mixture was warmed to room temperature and stirred for 30 minutes. Ethyl acetate (100 mL) and water (20 mL) were added and the organic layer washed with brine, dried over sodium sulfate and concentrated in vacuo providing 2.97 g of the title compound as a white solid. ¹H NMR (200 MHz, CDCl₃) δ 7.68 (dd, 1H), 6.69 (bs, 1H), 6.01 (bs, 1H), 2.48 (d, 3H).

g) 1-Cyclopropyl-3-(2,5-difluoro-4-iodo-3-methylbenzoyl)urea

A room temperature solution of 2,5-difluoro-4-iodo-3-methylbenzamide (2.97 g, 10 mmol, Example 25f) in 1,2-dichloroethane (20 mL) was treated dropwise with oxalyl chloride (3.80 g, 30 mmol). The mixture was stirred at room temperature for 1 hour, refluxed for 4 hours and concentrated in vacuo. The residue was dissolved in 40 mL of dioxane, cooled to 5° C. and treated dropwise with a solution of cyclopropylamine (1.14 g, 20 mmol) in dioxane (10 mL). The mixture was warmed slowly to room temperature, stirred for 3 hours and the solvent removed in vacuo. The residue was purified by column chromatography (1:100 ethyl acetate/chloroform) to provide the title compound (2.98 g). ¹H NMR (200 MHz, CDCl₃) δ 8.58-8.48 (m, 2H), 7.56 (dd, 1H), 2.78 (m, 1H), 2.49 (d, 3H), 0.83 (m, 2H), 0.65 (m, 2H).

h) 1-Cyclopropyl-6-fluoro-7-iodo-8-methyl-1H-quinazolinedione

To a 0° C. solution of 1-cyclopropyl-3-(2,5-difluoro-4-iodo-3-methylbenzoyl)urea (8.39 g, 22.1 mmol, Example 25g) in tetrahydrofuran (100 mL) and dimethylformamide (5 mL) was added, portionwise, sodium hydride (1.86 g, 77.3 mmol, 60% dispersion in mineral oil). The mixture was stirred at room temperature for 30 minutes, then refluxed 18 hours. After cooling, the mixture was poured onto ice, and the resulting solution acidified with 1N hydrochloric acid to pH 5. After extracting with ethyl acetate (500 mL), the organic layer was washed with brine, dried over sodium sulfate and concentrated in vacuo. The residue was purified by column chromatography (1:4 ethyl acetate/chloroform) to afford the title compound (4.20 g). ¹H NMR (200 MHz, CDCl₃) δ 8.64 (bs, 1H), 7.62 (d, 1H), 3.40 (m, 1H), 2.79 (s, 3H), 1.18 (m, 2H), 0.61 (m, 2H).

i) 5,6-Dihydrocyclopenta[b]thiophen-4-one Oxime

A mixture of 5,6-dihydrocyclopenta[b]thiophen-4-one (11.7 g, 85 mmol, [Russ. J. Org. Chem. 1998, 34(7), 1019]), hydroxylamine hydrochloride (9.03 g, 0.13 mol) and methanol (150 mL) was heated at 70° C. overnight. The solvent was removed in vacuo and the residue was dissolved in ethyl acetate (500 mL), washed with water, dried with sodium sulfate and concentrated in vacuo. The residue was purified by column chromatography (1:3 ethyl acetate/hexanes) to afford the title compound (10.7 g). ¹H NMR (400 MHz, DMSO-d₆) δ 10.42 (s, 1H), 7.56 (d, 1H), 7.00 (d, 1H), 3.12 (m, 2H), 3.02 (m, 2H); MSCI: m/z 154 (MH⁺).

j) 5,6-Dihydro-4H-cyclopenta[b]thiophen-4-ylamine

A mixture of 5,6-dihydrocyclopenta[b]thiophen-4-one oxime (10.0 g, 0.065 mol, Example 25i) and borane tetrahydrofuran complex (650 mL, 0.65 mol, 1 M in tetrahydrofuran) was refluxed for 18 hours. The reaction mixture was acidified with 4 N HCl and stirred at 70° C. for 1 hour. After cooling to room temperature, the mixture was washed with diethyl ether, and the aqueous phase was adjusted to pH 10 with 2N sodium hydroxide and extracted with ethyl acetate. The organic extracts were washed with brine, dried over sodium sulfate and concentrated in vacuo. The residue was then purified by column chromatography (9:1 dichloromethane/methanol) to afford the title compound (3.0 g). ¹H NMR (400 MHz, DMSO-d₆) δ 7.34 (d, 1H), 6.92 (d, 1H), 4.18 (m, 1H), 3.30 (bs, 2H), 2.92 (m, 1H), 2.73 (m, 2H), 1.98 (m, 1H).

k) (5,6-Dihydro-4H-cyclopenta[b]thiophen-4-yl)tritylamine

A mixture of 5,6-dihydro-4H-cyclopenta[b]thiophen-4-ylamine (2.54 g, 18.3 mmol, Example 25j), triphenylmethyl chloride (5.60 g, 20.1 mmol), triethylamine (2.77 g, 27.4 mmol) and dichloromethane (150 mL) was stirred at room temperature for 18 hours. The mixture was diluted with dichloromethane (200 mL), washed with brine, dried with sodium sulfate and concentrated in vacuo. The residue was purified by column chromatography (1:30 ethyl acetate/hexanes) to afford the title compound (6.90 g). ¹H NMR (400 MHz, CDCl₃) δ 7.65-7.20 (m, 15H), 7.08 (d, 1H), 6.39 (d, 1H), 4.18 (m, 1H), 2.76 (m, 1H), 2.52 (m, 1H), 2.00 (m, 1H), 1.96 (bs, 1H), 1.68 (m, 1H).

l) 1-Cyclopropyl-6-fluoro-8-methyl-7-[4-(tritylamino)-5,6-dihydro-4H-cyclopenta[b]thiophen-2-yl]-1H-quinazolinedione

A −78° C. solution of (5,6-dihydro-4H-cyclopenta[b]thiophen-4-yl)tritylamine (1.6 g, 4.2 mmol, Example 25k) in tetrahydrofuran (50 mL) was treated dropwise with n-butyllithium (4.2 mL, 10.5 mmol, 2.5 M in hexane), warmed to −10° C. and stirred for 3 hours. The reaction was cooled to −78° C., treated dropwise with a solution of n-tributyltin chloride (1.64 g, 5.04 mL) in tetrahydrofuran (5 mL) and allowed to warm to room temperature. After partitioning between ethyl acetate and water, the aqueous layer was extracted with ethyl acetate and the organic layers combined and washed with brine, dried over sodium sulfate and concentrated in vacuo. The resulting stannane was used immediately without purification by dissolving it in toluene (100 mL) and treating with 1-cyclopropyl-6-fluoro-7-iodo-8-methyl-1H-quinazolinedione (0.49 g, 1.36 mmol, Example 25h), triphenylarsine (0.165 g, 0.54 mmol), dichlorobis(triphenylphosphine)palladium(II) (0.098 g, 0.14 mmol), and copper(I) iodide (0.027 g, 0.14 mmol). The mixture was heated under a nitrogen atmosphere at 95° C. for 24 hours., then cooled to room temperature. The mixture was treated with ethyl acetate (500 mL) and 15% potassium fluoride (20 mL) and stirred at room temperature for 1 hour, then filtered though Celite. The aqueous layer was extracted with ethyl acetate (2×100 mL) and the combined organic extracts washed with brine, dried over sodium sulfate and concentrated in vacuo. The residue was purified by column chromatography (3:7 ethyl acetate/hexanes) to afford the title compound (0.48 g); ¹H NMR (400 MHz, CDCl₃) δ 8.22 (bs, 1H), 7.76 (d, 1H), 7.68-7.18 (m, 15H), 6.10 (s, 1H), 4.24 (m, 1H), 3.40 (m, 1H), 2.88 (m, 1H), 2.62 (m, 1H), 2.49 (s, 3H), 2.18 (m, 1H), 1.86 (m, 1H), 1.24 (m, 2H), 0.76 (m, 2H).

m) 7-(4-Amino-5,6-dihydro-4H-cyclopenta[b]thiophen-2-yl)-1-cyclopropyl-6-fluoro-8-methyl-1H-quinazolinedione Hydrochloride

Hydrogen chloride gas was bubbled through a 0° C. solution of 1-cyclopropyl-6-fluoro-8-methyl-7-[4-(tritylamino)-5,6-dihydro-4H-cyclopenta[b]thiophen-2-yl]-1H-quinazolinedione (0.48 g, 0.78 mmol, Example 25l) in diethyl ether (80 mL) and methanol (40 mL) for 30 minutes. After stirring at room temperature overnight, the solvent was removed in vacuo and the residue chromatographed on a silica gel column eluting with dichloromethane/methanol (85:15) to give the title compound (0.21 g): mp 233-235° C.; ¹H NMR (400 MHz, DMSO-d₆) δ 7.59 (d, 1H), 7.17 (s, 1H), 4.56 (m, 1H), 3.36 (m, 1H), 3.15 (m, 1H), 2.96 (m, 1H), 2.82 (m, 1H), 2.48 (s, 3H), 2.24 (m, 1H), 1.04 (m, 2H), 0.62 (m, 2H). MSCI: m/z 372 (MH⁺), 355 (MH⁺-NH₃).

### EXAMPLE 26

a) {1-[(R)-1-(1-cyclopropyl-6-fluoro-8-methyldioxo-1,2,3,4-tetrahydroquinazolin-7-yl)pyrrolidin-3-yl]cyclopropyl}carbamic Acid Tert-Butyl Ester

A solution of 1-cyclopropyl-6,7-difluoro-8-methyl-1H-quinazolinedione (0.50 g, 2.0 mmol), ((R)-1-pyrrolidin-3-ylcyclopropyl)carbamic acid tert-butyl ester (0.38 g, 1.7 mmol [U.S. Pat. No. 5,849,757]), 1,1,3,3-tetramethylguanidine (0.39 g, 3.4 mmol) and dimethyl sulfoxide (0.7 mL) was heated in a sealed tube at 75-80° C. for 80 hours. The mixture was cooled, diluted with water and extracted with ethyl acetate. The combined organic extracts were dried with sodium sulfate, filtered and concentrated in vacuo. The residue was purified by preparative thin layer chromatography (8:92 methanol/dichloromethane) to afford the title compound (0.23 g): ¹H NMR (400 MHz, CDCl₃) δ 9.00 (bs, 1H), 7.50 (d, 1H), 5.05 (bs, 1H), 3.70-3.58 (m, 1H), 3.55-3.46 (m, 1H), 3.40-3.26 (m, 3H), 2.35 (s, 3H), 1.81-1.68 (m, 1H), 1.50 (m, 2H), 1.41 (s, 9H), 1.25-1.18 (m, 1H), 1.11-1.00 (m, 1H), 0.90-0.51 (m, 6H).

b) 7-[(R)-3-(1-Aminocyclopropyl)pyrrolidin-1-yl]-1-cyclopropyl-6-fluoro-8-methyl-1 h-quinazolinedione Hydrochloride

Hydrogen chloride gas was bubbled through a 0° C. solution of {1-[(R)-1-(1-cyclopropyl-6-fluoro-8-methyldioxo-1,2,3,4-tetrahydroquinazolin-7-yl)pyrrolidin-3-yl]cyclopropyl}carbamic acid tert-butyl ester (0.23 g, 0.51 mmol, Example 26a) in anhydrous diethyl ether (15 mL) for 10 minutes. The resulting suspension was slowly warmed to room temperature and stirred for 5 hours. The solid was removed by filtration, washed with dichloromethane (10 mL) and dried in vacuo to afford the title compound (0.12 g): mp 202-203° C.; ¹H NMR (400 MHz, DMSO-d₆) δ 8.52 (bs, 3H), 7.38 (d, 1H), 3.60-3.50 (m, 1H), 3.45-3.10 (m, 4H), 2.70-2.60 (m, 1H), 2.35 (s, 3H), 2.10-1.96 (m, 1H), 1.70-1.60 (m, 1H), 1.10-0.82 (m, 6H), 0.60-0.45 (m, 2H).

### EXAMPLE 27

a) 4,5,6,7-Tetrahydrobenzo[b]thiophen-7-yl)carbamic Acid Tert-Butyl Ester

Di-tert-butyldicarbonate (0.79 g, 3.6 mmol) was added to a room temperature solution of 4,5,6,7-tetrahydrobenzo[b]thiophen-7-ylamine (0.37 g, 2.4 mmol [Eur. J. Med. Chem. Chim. Ther. 1998, 33, 867], triethylamine (0.41 g, 4.0 mmol) and dry diethyl ether (15 mL). After stirring for 1 hour, the reaction mixture was diluted with diethyl ether, washed with water, 2 N hydrochloric acid, saturated bicarbonate solution and brine. The diethyl ether solution was dried with sodium sulfate, concentrated in vacuo and the residue purified by flash chromarography on silica gel (hexane/ethyl acetate 10:1) to provide the title compound (0.59 g) as a colorless solid. ¹H NMR (400 MHz, CDCl₃) δ 7.12 (d, 1H), 6.73 (d, 1H), 4.98-4.48 (m, 2H), 2.70-2.52 (m, 2H), 2.18-2.04 (m, 1H), 1.91-1.80 (m, 3H), 1.47 (s, 9H).

b) (2-Tributylstannanyl-4,5,6,7-tetrahydrobento[b]thiophen-7-yl)carbamic Acid, Tert-Butyl Ester

A −30° C. solution of (4,5,6,7-tetrahydrobenzo[b]thiophen-7-yl)carbamic acid tert-butyl ester (0.127 g, 0.5 mmol, Example 27a) in anhydrous tetrahydrofuran (3 mL) was treated dropwise with n-butyllithium (0.5 mL of a 2.5 M hexane solution, 1.25 mmol) under a nitrogen atmosphere. After stirring for 1 h at −30° C., the mixture was cooled to −70° C., treated with neat tri-n-butyltin chloride (407 mg, 1.25 mmol) and allowed to warm to 0° C. The reaction mixture was diluted with diethyl ether and water and the organic layer was washed with water, brine, dried over sodium sulfate and concentrated under vacuum. Purification of the residue by flash chromatography on silica gel (hexane/ethyl acetate/triethylamine 200:10:1) gave the title compound (0.180 g). ¹H NMR (400 MHz, CDCl₃) δ 6.79 (s, 1H), 4.98-4.50 (m, 2H), 2.71-2.53 (m, 2H), 2.15-2.03 (m, 1H), b 1.90-1.70 (m, 3H), 1.62-1.50 (m, 6H), 1.47 (s, 9H), 1.38-1.28 (m, 6H), 1.10-1.03 (m, 6H), 0.89 (t, 9H).

c) [2-(1-cyclopropyl-6-fluoro-8-methyldioxo-1,2,3,4-tetrahydroquinazolin-7-yl)-4,5,6,7-tetrahydrobenzo[b]thiophen-7-yl]carbamic Acid Tert-Butyl Ester

A mixture of (2-tributylstannanyl-4,5,6,7-tetrahydrobenzo[b]thiophen-7-yl)carbamic acid tert-butyl ester (0.180 g, 0.33 mmol, Example 27b), 1-cyclopropyl-6-fluoro-7-iodo-8-methyl-1H-quinazolinedione (0.119 g, 0.33 mmol, Example 25h), dichlorobis(triphenylphosphine) palladium (II) (0.023 g, 0.033 mmol), and triphenylarsine (0.031 g, 0.11 mmol) in anhydrous toluene (6 mL) was stirred under nitrogen at 95° C. for 20 hours. After cooling to room temperature, diethyl ether (15 mL) was added, followed by 15% aqueous potassium fluoride solution and stirring was continued for 1 hour at room temperature. The mixture was filtered through Celite and the organic layer was washed with water, dried with sodium sulfate and concentrated in vacuo. Purification by flash chromatography on silica gel (hexane/ethyl acetate 1:1) gave the title compound (102 mg) as colorless crystals. ¹H NMR (400 MHz, CDCl₃) δ 8.28 (bs, 1H), 7.67 (d, 1H), 6.72 (s, 1H), 5.02-4.62 (m, 2H), 3.38 (m, 1H), 2.71-2.68 (m, 2H), 2.51 (s, 3H), 2.22-2.10 (m, 1H), 1.97-1.73 (m, 3H), 1.47 (s, 9H), 1.21-1.13 (m, 2H), 0.73-0.63 (m, 2H).

d) 7-(4-Amino-5,6-dihydro-4H-4,5,6,7-tetrahydrobenzo[b]thiophen-7-yl)-1-cyclopropyl-6-fluoro-8-methyl-1H-quinazolinedione Hydrochloride

A stream of gaseous hydrogen chloride was bubbled through a 0° C. solution of 2-(1-cyclopropyl-6-fluoro-8-methyldioxo-1,2,3,4-tetrahydroquinazolin-7-yl)-4,5,6,7-tetrahydrobenzo[b]thiophen-7-yl]carbamic acid tert-butyl ester (0.102 g, 0.21 mmol, Example 27c) in anhydrous diethyl ether (10 mL) for 40 minutes. The resulting precipitate was removed by filtration, washed with anhydrous diethyl ether and dried in vacuo to provide the title compound (0.036 g): mp>220° C. (dec.); ¹H NMR (400 MHz, DMSO-d₆) δ 11.55 (bs, 1H), 8.40 (bs, 3H), 7.57 (d, 1H), 7.01 (s, 1H), 4.58 (m, 1H), 3.30 (m, 1H), 2.75-2.65 (m, 2H), 2.40 (s, 3H), 2.18-2.05 (m, 1H), 2.02-1.90 (m, 2H), 1.87-1.70 (m, 1H), 1.03 (m, 2H), 0.58 (m, 2H).

### EXAMPLE 28

a) 7-Methyl-6-trityl-4,5,6,7-tetrahydrothieno[2,3-c]pyridine

A solution of 7-methyl-4,5,6,7-tetrahydrothieno[2,3-c]pyridine (1.50 g, 9.94 mmol [J. Med Chem.1989, 32, 1242.]) in dichloromethane (20 mL) was treated, portionwise, with trityl bromide (3.86 g, 11.9 mmol) at room temperature followed by triethylamine (2.20 mL, 15.8 mmol). The reaction mixture was stirred at room temperature for 4 hours, washed with water (2×40 mL), dried over sodium sulfate, filtered, and the solvent removed under reduced pressure. The residue was chromatographed on silica gel (ethyl acetate/hexane 5:95) to provide 3.45 g of the title compound. ¹H NMR (400 MHz, CDCl₃) δ 7.50-7.07 (m, 15H), 6.90 (d, 1H), 6.33 (d, 1H), 4.60 (q, 1H), 3.50-3.33 (m, 2H), 2.03-1.95 (m, 1H), 1.63-1.53 (m, 1H), 1.36 (d, 3H).

b) 7-Methyl-2-tri-n-butylstannanyl-6-trityl-4,5,6,7-tetrahydrothieno[2,3-cipyridine

Under a nitrogen atmosphere, a −78° C. solution of 7-methyl-6-trityl-4,5,6,7-tetrahydro-thieno[2,3-c]pyridine (0.11 g, 0.26 mmol, Example 28a) in anhydrous tetrahydrofuran (0.50 mL) was treated with n-butyllithium (0.20 mL of a 2.5M hexane solution, 0.50 mmol) and stirred at −78° C. for one hour. Tri n-butylstannyl chloride (0.1 mL, 0.37 mmol) was then added and the reaction mixture was stirred at −78° C. for one hour then allowed to come to room temperature over 4.5 hours. The reaction was quenched with methanol (3 mL) and concentrated under reduced pressure. The residue was chromatographed over silica gel eluting with ethyl acetate/hexane/triethylamine (5:95:0.5) to give 0.087 g of the title compound. ¹H NMR (400 MHz, CDCl₃) δ 7.55-7.03 (m, 15H), 6.37 (s, 1H), 4.70-4.58 (m, 1H), 3.52-3.33 (m, 2H), 2.05-1.93 (m, 1H), 1.63-1.17 (m, 13H), 1.07-0.82 (m, 18H).

c) 1-Cyclopropyl-6-fluoro-8-methyl-7-(7-methyl-6-trityl-4,5,6,7-tetrahydrothieno[2,3-c]pyridin-2-yl)-1H-quinazolinedione

Under a nitrogen atmosphere, a mixture of 7-methyl-2-tri-n-butylstannanyl-6-trityl-4,5,6,7-tetrahydrothieno[2,3-c]pyridine (1.86 g, 2.72 mmol, Example 28b), 1-cyclopropyl-6-fluoro-7-iodo-8-methyl-1H-quinazolinedione (0.448 g, 1.24 mmol, Example 25h), triphenylarsine (0.160 g, 0.522 mmol) and tris(dibenzylideneacetone)dipalladium(0) (0.115 g, 0.125 mmol) in anhydrous toluene (2 mL) was heated with stirring at 95° C. for 19 hours. After cooling to room temperature, the reaction mixture was diluted with ethyl acetate (20 mL) and 15% w/v aqueous potassium fluoride solution (15 mL) and stirred for 2 h. The mixture was filtered through Celite and washed with ethyl acetate. The organic phase was then dried over anhydrous sodium sulfate, filtered and concentrated under reduced pressure. The residue was purified by silica gel chromatography eluting with ethyl acetate/hexane/triethylamine gradient (30:70:1 to 50:50:1) and gave the title compound (0.551 g). ¹H NMR (400 MHz, CDCl₃) δ 8.07 (bs, 1H), 7.67 (d, 1H), 7.55-7.08 (m, 15H), 6.27 (s, 1H), 4.70-4.62 (m, 1H), 3.63-3.47 (m, 2H), 3.42-3.32 (m, 1H), 2.47 (s, 3H), 2.02-1.93 (m, 2H), 1.55 (d, 3H), 1.23-1.13 (m, 2H), 0.73-0.63 (m, 2H).

d) 1-Cyclopropyl-6-fluoro-8-methyl-7-(7-methyl-4,5,6,7-tetrahydrothieno[2,3-c]pyridin-2-yl)-1H-quinazolinedione

Gaseous hydrogen chloride was bubbled through a 0° C. suspension of 1-cyclopropyl-6-fluoro-8-methyl-7-(7-methyl-6-trityl-4,5,6,7-tetrehydrothieno[2,3-c]pyridin-2-yl)-1H-quinazolinedione (0.551 g, 0.878 mmol, Example 28c) in diethyl ether for 25 minutes and then the mixture was warmed to room temperature and stirred overnight. The resulting solid was isolated by filtration, suspended in dichloromethane (10 mL), and treated with triethylamine (2 mL). The mixture was concentrated under reduced pressure and the residue was purified by column chromatography on silica gel eluting with dichloromethane/methanol/triethylamine (90:10:1). The product was then triturated with methanol to give the title compound (0.070 g). ¹H NMR (400 MHz, CD₃OD) δ 11.58 (s, 1H), 9.83 (bs, 1H), 7.58 (d, 1H), 7.10 (s, 1H), 4.83-4.70 (m, 1H), 3.65-3.52 (m, 1H), 3.45-3.30 (m, 2H), 3.02-2.90 (m, 2H), 2.45 (s, 3H), 1.65 (d, 3H), 1.12-0.98 (m, 2H), 0.75-0.55 (m, 2H); MSCI: m/z 386 (MH⁺).

### EXAMPLE 29

a) 1-(5-Bromothiophen-3-yl)ethanone

To a solution of 3-acetylthiophene (10.3 g, 82 mmol) in acetic acid (50 mL) was added sodium acetate (10.0 g, 122 mmol) followed by bromine (4.5 mL, 86 mmol) dropwise over 30 minutes. The mixture was allowed to stir at room temperature overnight. Water (150 mL) was added and the reaction mixture was stirred for 2 hours before the resulting solid was collected by filtration, washed with water, and hexane to give 7.67 g of the title compound. ¹H NMR (200 MHz, CDCl₃) δ 7.93 (d, 1H), 7.50 (d, 1H), 2.48 (s, 3H).

b) 1-(5-Bromothiophen-3-yl)ethanone O-benzyl Oxime

A solution of 1-(5-bromothiophen-3-yl)ethanone (2.1 g, 10 mmol, Example 29a) in methanol (20 mL) was treated with O-benzylhydroxylamine hydrochloride (1.76 g, 11 mmol) and refluxed for 3 hours. The solvent was removed in vacuo and the resulting residue purified by column chromatography (8:1 hexane/ethyl acetate) to give 2.8 g of the title compound. ¹H NMR (200 MHz, CDCl₃) δ 7.45-7.20 (m, 7H), 5.15 (s, 2H), 2.15 (s, 3H).

c) 1-(5-Bromothiophen-3-yl)ethylamine

A solution of 1-(5-bromothiophen-3-yl)ethanone O-benzyl oxime (2.7 g, 8.74 mmol, Example 29b) in tetrahydrofuran (30 mL) was treated with a solution of borane-tetrahydrofuran complex (20 mL, 1 M in THF) and heated at 50° C. for 24 hours. Methanol (25 mL) was added and the solvent removed in vacuo. The resulting residue was purified by column chromatography (5-10% methanol/chloroform) to give 0.88 g of the title compound. ¹H NMR (200 MHz, CDCl₃) δ 7.02 (m, 2H), 4.10 (q, 1H), 1.65 (bs, 2H), 1.37 (d, 3H).

d) 1-(5-Bromo-2-chloromethylthiophen-3-yl)ethylamine Hydrochloride

A solution of 1-(5-bromothiophen-3-yl)ethylamine (2.05 g, 10 mmol, Example 29c) in concentrated hydrochloric acid (30 mL) was treated with paraformaldehyde (1.0 g, 33 mmol) and the reaction mixture was stirred at room temperature for 3 hours. After cooling to 5° C. and stirring for 2 hours, the resulting solid was collected by filtration and washed with small amounts of concentrated hydrochloric acid to give 1.84 g of the title compound. ¹H NMR (200 MHz, DMSO-d₆) δ 8.64 (bs, 3H), 7.54 (s, 1H), 5.12 (q, 2H), 4.61 (m, 1H), 1.48 (d, 3H).

e) 2-Bromo-4-methyl-4,6-dihydrothieno[2,3-c]pyrrole-5-carboxylic Acid Tert-Butyl Ester

A suspension of 1-(5-bromo-2-chloromethylthiophen-3-yl)ethylamine hydrochloride (1.84 g, 6.35 mmol, Example 29d) in tetrahydrofuran (80 mL) was treated with triethylamine (2 mL) and stirred at room temperature for 2 hours. Di-tert-butyl dicarbonate (1.66 g, 7.6 mmol) was added and the reaction mixture was stirred at room temperature for 18 hours. The solid was removed by filtration, the filtrate concentrated in vacuo and the residue purified by silica column chromatography eluting with hexane/ethyl acetate (16:1 to 8:1) to give 1.5 g of the title compound. ¹H NMR (200 MHz, CDCl₃) δ 6.80 (d, 1H), 5.00-4.80 (m, 1H), 4.70-4.40 (m, 2H), 1.60-1.30 (m, 12H).

f) 4-Methyl-2-tributylstannanyl-4,6-dihydrothieno[2,3-c]pyrrole-5-carboxylic Acid, Tert-Butyl Ester

Under a nitrogen atmosphere, to a −78° C. solution of 2-bromo-4-methyl-4,6-dihydro-thieno[2,3-c]pyrrole-5-carboxylic acid tert-butyl ester (1.5 g, 4.7 mmol, Example 29e) in diethyl ether (50 mL) was added n-butyllithium (2.5 M, 5 mL, 12.5 mmol) and the mixture was allowed to stir for 10 minutes. Tri-n-butylstannyl chloride (3.5 g, 10.5 mmol) was then added and after stirring at −78° C. for 40 minutes, methanol (30 mL) was added and the solvent removed in vacuo. The residue was partitioned between ethyl acetate and water (100 mL each), and the organic layer washed with water, dried with sodium sulfate and concentrated in vacuo. The residue was purified by column chromatography (16:1 hexane/ethyl acetate with 0.5% triethylamine) to give 2.3 g of the title compound.

¹H NMR (200 MHz, CDCl₃) δ 6.80 (d, 11H), 4.80-5.00 (m, 1H), 4.70-4.50 (m, 2H), 1.60-0.60 (m, 39H).

g) 2-(1-Cyclopropyl-6-fluoro-8-methyldioxo-1,2,3,4-tetrahydroquinazolin-7-yl)-4-methyl-4,6-dihydrothieno[2,3-c]pyrrole-5-carboxylic Acid Tert-Butyl Ester

To a mixture of 4-methyl-2-tributylstannanyl-4,6-dihydrothieno[2,3-c]pyrrole-5-carboxylic acid tert-butyl ester (0.96 g, 1.82 mmol, Example 29f) and 1-cyclopropyl-6-fluoro-7-iodo-8-methyl-1H-quinazolinedione (0.275 g, 0.76 mmol, Example 25h) in toluene (8 mL) was added dichlorobis(triphenylphosphine)palladium(II) (0.053 g, 0.076 mmol) and triphenylarsine (0.097 g, 30 mmol). The mixture was heated at 100° C. in a sealed tube for 4 hours. The mixture was concentrated in vacuo and the residue purified by column chromatography (2:1 hexane/ethyl acetate, and 1:1 hexane/ethyl acetate) to give 0.33 g of the title compound. ¹H NMR (200 MHz, CDCl₃) δ 8.86 (bs, 1H), 7.74 (d, 1H), 6.82 (d, 1H), 5.10-4.90 (m, 1H), 4.80-4.60 (m, 2H), 3.39 (m, 1H), 2.54 (s, 3H), 1.53 (m, 12H), 1.30-1. 10 (m, 2H), 0.68 (m, 2H).

h) 1-Cyclopropyl-6-fluoro-8-methyl-7-(4-methyl-5,6-dihydro-4H-thieno[2,3-c]pyrrol-2-yl)-1H-quinazolinedione Hydrochloride

A stream of hydrogen chloride gas was bubbled into a solution of 2-(1-cyclopropyl-6-fluoro-8-methyldioxo-1,2,3,4-tetrahydroquinazolin-7-yl)-4-methyl-4,6-dihydrothieno[2,3-c]pyrrole-5-carboxylic acid tert-butyl ester (0.30 g, 0.637 mmol, Example 29g) in a solvent mixture of dichloromethane (10 mL) and diethyl ether (25 mL). The resulting solution was cooled to 0-5° C. for 1 hour and then stirred at room temperature for 2 hours. The solid was collected by filtration and washed with diethyl ether to provide 0.235 g of the title compound. ¹H NMR (200 MHz, DMSO-d₆) δ 11.60 (s, 1H), 10.54 (bs, 1H), 10.01 (bs, 1H), 7.60 (d, 1H), 7.17 (s, 1H), 4.92 (m, 1H), 4.58 (m, 2H), 3.33 (m, 1H), 2.46 (s, 3H), 1.61 (d, 3H), 0.90-1.04 (m, 2H), 0.64 (m, 2H). MSCI: m/z 371 (MH⁺).

### EXAMPLE 30

a) (3S, 3aS, 6aR and 3R, 3aR, 6aS)-2-Benzyl-3-trifluoromethylhexahydro-pyrrolo[3,4-d]isoxazole-5-carboxylic Acid Benzyl Ester

A mixture of N-benzylhydroxylamine (1.6 g, 10 mmol), 1-ethoxy-2,2,2-trifluoromethyl-ethanol (1.6 g, 90%, 10 mmol ) and triethylamine (1.5 mL) in benzene (50 mL) was refluxed for 2 hours. After cooling to room temperature, 2,5-dihydropyrrole-1-carboxylic acid benzyl ester (2.0 g, 10 mmol) was added and the reaction mixture was refluxed for 24 hours. The solvent was removed in vacuo and the residue triturated with hexane/ethyl acetate (100 mL of a 2:1 mixture). The solid was removed by filtration and the filtrate concentrated under reduced pressure. The residue was purified by column chromatography (8:1 hexane/ethyl acetate) to give 3.2 g of the title compound. ¹H NMR (400 MHz, CDCl₃) δ 7.20 (m, 10H), 5.20 (s, 2H), 4.65 (m, 1H), 4.30 (m, 1H), 4.05 (m, 1H), 3.75 (m, 2H), 3.10-3.50 (m, 4H).

b) ([(3R, 4S)-4-(R)- and (3S, 4R)-4-(S)]-1-Amino-2,2,2-trifluoroethyl)pyrrolidin-3-ol

A suspension of (3S, 3aS, 6aR)- and (3R, 3aR, 6aS)-2-benzyl-3-trifluoromethylhexahydropyrrolo[3,4-d]isoxazole-5-carboxylic acid benzyl ester (1.7 g, 4.18 mmol, Example 30a) and 1.0 g of palladium on carbon (10% Pd, 50% water) in methanol (70 mL) was shaken under a hydrogen atmosphere at 50 psi for 20 hours. The catalyst was removed by filtration and the solvent removed in vacuo to give 0.78 g of the title compound. ¹H NMR (400 MHz, DMSO-d₆) δ 4.40 (m, 1H), 3.30 (m, 1H), 3.20-2.80 (m, 4H), 2.10 (m, 1H). MSCI: m/z 184 (MH⁺).

c) 7-[[(3S, 4R)-3-(R)- and (3R, 4S)-3-(S)]-1-amino-2,2,2-trifluoroethyl)-4-hydroxypyrrolidin-1-yl]-1-cyclopropyl-6-fluoro-8-methyl-1H-quinazolinedione

A mixture of ([(3R, 4S)-4-(R)- and (3S, 4R)-4-(S)]-1-amino-2,2,2-trifluoroethyl)pyrrolidin-3-ol (0.70 g, 3.8 mmol, Example 30b), 1-cyclopropyl-6,7-difluoro-8-methyl-1H-quinazolinedione (0.90 g, 3.6 mmol) and triethylamine (0.5 mL) in 5 mL of dimethyl sulfoxide was heated at 10° C. for 40 hours. The reaction mixture was then diluted with 50 mL of ethyl acetate and 50 mL of water. The organic layer was separated and the water layer was extracted with ethyl acetate (50 mL×3). The combined organic layers were washed with water. The solvent was then removed in vacuo and the residue purified by column chromatography (5% methanol in chloroform, then 10% methanol in chloroform) to give 0.518 g of the title compound. ¹H NMR (400 MHz, DMSO-d₆) δ 11.25 (s, 1H), 7.25 (d, 1H), 5.10 (d, 11H), 4.50 (m, 1H), 3.90 (d, 11H), 3.75 (m, 11H), 3.40 (m, 1H), 3.30 (m, 1H), 3.25 (m, 2H), 3.10 (d, 1H), 2.28 (s, 3H), 2.20 (m, 1H), 2.10 (s, 1H), 1.10 (m, 1H), 1.00 (m, 1H), 0.58 (m, 1H), 0.52 (m, 1H). ¹⁹F NMR (376 MHz, DMSO-d₆) 6-75 (s, 3F), −129 (s, 1F). MSCI: m/z 416(MH⁺).

### EXAMPLE 31

a) 4-(Oxazole-4-carbonyl)-1-((S)-1-phenylethyl)pyrrolidin-2-one

To a −78° C. solution of oxazole (10.30 g, 149.10 mmol) in tetrahydrofuran (150 mL) was added n-butyl lithium (2.5 M in hexane, 53.7 mL, 134.19 mmol). The solution was stirred at −78° C. for 3 hours and treated with a solution of 5-oxo-1-((S)-1-phenylethyl)-pyrrolidine-3-carboxylic acid methoxymethylamide (8.24 g, 29.8 mmol, Example 7a) in tetrahydrofuran (50 mL). The reaction was allowed to warm to room temperature and stir for an additional 3 hours. Water was added followed by saturated ammonium chloride solution, and the mixture extracted with ethyl acetate. The combined organic layers were dried over sodium sulfate, filtered and concentrated under reduced pressure. The residue was chromatographed over silica gel (hexanes/ethyl acetate, 1:4) to afford 1.96 g of the title compound as a mixture of isomers (˜1:1 ratio). 1^{st} Isomer ¹H NMR (400 MHz, CDCl₃) δ 7.88 (s, 1H), 7.40-7.21 (m, 6H), 5.50 (q, 1H), 4.18-4.10 (m, 1H), 3.66-3.62 (m, 1H), 3.35-3.28 (m, 1H), 2.95-2.75 (m, 2H), 1.52 (d, 3H). 2^{nd} Isomer ¹H NMR (400 MHz, CDCl₃) δ 7.85 (s, 11H), 7.40-7.21 (m, 6H), 5.50 (q, 1H), 4.30-4.20 (m, 1H), 3.78-3.67 (m, 1H), 3.29-3.18 (m, 1H), 2.95-2.75 (m, 2H), 1.55 (d, 3H).

b) 4-(Benzyloxyiminooxazol-4-ylmethyl)-1-((S)-1-phenylethyl)-pyrrolidin-2-one

A mixture of 4-(oxazole-4-carbonyl)-1-((S)-1-phenylethyl)pyrrolidin-2-one (0.5 g, 1.76 mmol, Example 31a) and O-benzylhydroxylamine hydrochloride (0.42 g, 2.64 mmol) in pyridine (5 mL) was refluxed for 5 hours and cooled to room temperature. The mixture was diluted with water and extracted with ethyl acetate (2×20 ml). The combined organic layers were washed with saturated sodium bicarbonate solution, dried with sodium sulfate, filtered and concentrated in vacuo. The residue was chromatographed over silica gel, eluting with hexanes:ethyl acetate (1:2), to give 0.41 g of the title compound as a mixture of isomers. ¹H NMR (400 MHz, CDCl₃) δ 7.75-7.60 (m, 1H), 7.40-7.12 (m, 11H), 5.60-5.45 (m, 1H), 5.30-5.05 (m, 2H), 4.30-4.16 (m, 1H), 3.88-2.60 (m, 4H), 1.50, 1.49, 1.45-1.30 (m, 3H).

c) C-Oxazol-4-yl-C-[1-((S)-1-phenylethyl)pyrrolidin-3-yl]methylamine

To a 0° C. solution of 4-(benzyloxyiminooxazol-4-ylmethyl)-1-((S)-1-phenylethyl)-pyrrolidin-2-one (3.16 g, 8.11 mmol, Example 31 b) in tetrahydrofuran (80 mL) was added a 1.0 M solution of borane-tetrahydrofuran complex (24.3 mL, 24.3 mmol) and the reaction stirred at room temperature for 21 hours. The solvent was evaporated and the residue was taken up in water (5 mL) and extracted with chloroform. The combined organic layers were evaporated under reduced pressure and the residue dissolved in 80% aqueous ethanol, treated with triethylamine (20 mL) and then heated to reflux for two hours. The mixture was concentrated to remove the organic solvents and the aqueous mixture extracted with dichloromethane. The organic extracts were then combined, dried over sodium sulfate and concentrated in vacuo. The resulting residue was purified using silica gel column chromatography (chloroform/methanol, 9:1) to obtain the title compound (2.5 g) as a mixture of isomers. ¹H NMR (400 MHz, CDCl₃) δ 7.55-7.54 (2 m, 1H), 7.40-7.15 (m, 7H), 7.10-7.05 (m, 1H), 4.02-3.88 (m, 1H), 3.25-3.10 (m, 1H), 2.90-2.20 (m, 5H), 2.00-1.60 (m, 2H), 1.60-1.35 (m, 3H).

d) C-Oxazol-4-yl-C-pyrrolidin-3-ylmethylamine

A mixture of C-oxazol-4-yl-C-[1-((S)-1-phenylethyl)pyrrolidin-3-yl]methylamine (2.44 g, 8.99 mmol, Example 31c), ammonium formate (2.83 g, 45.0 mmol) and 10% palladium on carbon (2.87 g) in methanol (45 mL) was heated at reflux for 5 hours. After filtering through Celite, the filtrate was concentrated under reduced pressure to obtain the title compound (0.9 g). ¹H NMR (400 MHz, CDCl₃) δ 7.66-7.64 (m, 1H), 7.13-7.11(m, 1H), 4.18-4.10 (m, 1H), 3.50-3.40 (bs, 3H), 3.40-3.25 (m, 3H), 3.08-2.80 (m, 2H), 2.30-2.05 (m, 1H), 1.90-1.60 (m, 1H).

e) 7-[3-(Aminooxazol-4-ylmethyl)pyrrolidin-1-yl]-1-cyclopropyl-6-fluoro-8-methyl-1H-quinazolinedione

The title compound was obtained as a white solid (0.21 g) from 1-cyclopropyl-6,7-difluoro-8-methyl-1H-quinazolinedione (0.66 g, 2.60 mmol) and C-oxazol-4-yl-C-pyrrolidin-3-ylmethylamine (1.09 g, 6.5 mmol, Example 31d) according to the method described for Example 2. ¹H NMR (400 MHz, CDCl₃) δ 7.63-7.61 (m, 1H), 7.60-7.45 (m, 1H), 7.13-7.11 (m, 1H), 4.13-4.10 (m, 1H), 3.60 (bs, 3H), 3.35-3.25 (m, 2H), 2.81-2.68 (bs, 3H), 2.41-2.39 (m, 3H), 2.30-1.86 (m, 3H), 1.25-1.05 (m, 2H), 0.60 (bs, 2H). MSCI: m/z 400 (MH⁺)

### EXAMPLE 32

a) [(3R,4S)- and (3S, 4R)-1-(1-Cyclopropyl-6-fluoro-8-methoxydioxo-1,2,3,4-tetrahydroquinazolin-7-yl)-4-fluoropyrrolidin-3-ylmethyl]carbamic Acid Tert-Butyl Ester

The title compound, as a mixture of two isomers (2:1), was obtained as a white solid (0.052 g) from 1-cyclopropyl-6,7-difluoro-8-methoxy-1H-quinazolinedione (0.49 g, 1.832 mmol) and (3S, 4S) and ((3R, 4R)-4-fluoropyrrolidin-3-ylmethyl)carbamic acid tert-butyl esters (0.60 g, 2.747 mmol, [J. Med. Chem. 1990, 33, 1344]). ¹H NMR (400 MHz, CDCl₃) δ 9.10-8.82 (bs, 1H), 7.47 (m, 1H), 5.12 (d, 1H), 5.00-4.85 (bm, 1H), 4.10-4.05 (m, 1H), 3.73-3.60 (m, 2H) 3.52-3.48 (m, 3H), 3.32-3.08 (m, 2H), 2.72-2.60 (m, 1H), 2.50-2.35, 2.15-2.00, 1.75-1.65 & 1.65-1.50 (4×m, 2H), 1.46 (s, 9H), 1.25-0.95 (m, 2H), 0.75-0.45 (m, 2H). MSCI: m/z 467 (MH⁺).

b) 7-(3R, 4S)- and 7-((3S, 4R)-3-Aminomethyl-4-fluoropyrrolidin-1-yl)-1-cyclopropyl-6-fluoro-8-methoxy-1H-quinazoloinedione Hydrochloride

A solution of (3R,4S) and [(3S, 4R)-1-(1-cyclopropyl-6-fluoro-8-methoxydioxo-1,2,3,4-tetrahydroquinazolin-7-yl)-4-fluoropyrrolidin-3-ylmethyl]carbamic acid tert-butyl ester (0.052 g, 0.112 mmol, Example 32a) in dichloromethane was saturated with hydrogen chloride gas and stirred at room temperature for 18 hours. The solvent was removed under reduced pressure to give the title compound as a white solid in a 2:1 mixture isomers (0.045 g). ¹H NMR (400 MHz, CD₃OD) δ 7.40 (m, 1H), 5.25 (d, 1H), 4.23-3.96 (m, 2H), 3.85-3.64 (2×m, 2H), 3.62 (s, 3H), 3.51-3.48 (m, 1H), 2.85-2.72 (m, 1H), 2.65-2.55, 2.30-2.20, 1.85-1.70 &1.50-1.35 (4×m, 2H), 1.15-0.88 (m, 2H), 0.72-0.56 (m, 2H). MSCI: m/z 367 (MH⁺).

### EXAMPLE 33

a) 5-benzyl-3-(tetrahydropyran-2-yloxymethyl)-4,5,6,6a-tetrahydro-3aH-pyrrolo[3,4-d]isoxazole

To a solution of 1-benzyl-2,5-dihydro-1H-pyrrole (13.5 g, 84.8 mmol) in benzene (150 mL) was added 2-(2-nitroethoxy)tetrahydropyran (37 g, 211.2 mmol) and triethylamine (5.4 mL, 38.4 mmol). The solution was heated to reflux and phenyl isocyanate (37.8 mL, 347.8 mmol) was slowly added over 2 hours. After the addition was complete, the mixture was refluxed overnight and the resulting precipitate removed by filtration. The filtrate was concentrated in vacuo and the residue purified by column chromatography eluting with ethyl acetate:hexanes (1:4), to obtain 19.5 g the title compound. ¹H NMR (200 MHz, CDCl₃) δ 7.38-7.18 (m, 5H), 5.10-4.98 (m, 1H), 4.68-4.58 (bs, 1H), 4.50-4.18 (m, 2H), 3.86-3.42 (m, 5H), 3.25-3.05 (m, 2H), 2.44-2.24 (rA, 2H), 1.82-1.38 (m, 6H).

b) 4-[1-Amino-2-(tetrahydropyran-2-yloxyethyl]-1-benzylpyrrolidin-3-ol

To a 5° C. solution of 5-benzyl-3-(tetrahydropyran-2-yloxymethyl)-4,5,6,6a-tetrahydro-3aH-pyrrolo[3,4-d]isoxazole (8.80 g, 27.8 mmol, Example 33a) in diethyl ether (150 mL) was added, portionwise, lithium aluminum hydride (2.58 g, 68.0 mmol). The mixture was stirred at 5° C. for 1 hour and then at room temperature for 1 hour. The mixture was then recooled to 5° C. and treated successively, dropwise, with water (2.6 mL), 3N sodium hydroxide (2.6 mL) and water (7.7 mL). The mixture was then diluted with chloroform and stirred at room temperature for 2 hours. The mixture was filtered through Celite, and the filter cake was washed copiously with chloroform. The combined filtrates were dried over sodium sulfate and evaporated. The residue was triturated with diethyl ether, the solid removed by filtration, washed with ether and dried in vacuo to give 7.3 g of the title compound. ¹H NMR (200 MHz, CDCl₃) δ 7.35-7.28 (m, 5H), 4.62-4.52 (bs, 1H), 4.46-4.35 (bs, 1H), 3.86-3.12 (m, 8H), 3.00-2.88 (m, 1H), 2.65-2.48 (m, 2H), 2.35-2.18 (m, 1H) 1.80-1.40 (m, 9H).

c) [1-(1-Benzyl-4-hydroxypyrolidin-3-yl)-2-(tetrahydropyran-2-yloxy)ethyl]-carbamic Acid Tert-Butyl Ester

To a solution of 4-[1-amino-2-(tetrahydropyran-2-yloxyethyl]-1-benzylpyrrolidin-3-ol (7.3 g, 22.8 mmol, Example 33b) in chloroform (60 mL) was added di-tert-butyl dicarbonate (4.97 g, 22.8 mmol). The mixture was stirred at room temperature for 3 hours, and the solvent was removed in vacuo. The residue was purified by column chromatography on silica gel (5% methanol in ethyl acetate) to obtain 7.8 g of the title compound. ¹H NMR (200 MHz, CDCl₃) δ 7.32-7.25 (m, 5H), 5.15-4.85 (m, 1H), 4.62-4.58 (bs, 1H), 4.36-4.20 (m, 1H), 4.00-3.35 (m, 7H), 3.26-3.05 (bs, 1H), 3.00-2.88 (m, 1H), 2.70-2.28 (m, 4H), 1.80-1.48 (m, 6H), 1.42 (s, 9H).

d) [1-(1-Benzyl-4-hydroxypyrrolidin-3-yl)-2-hydroxyethyl]carbamic Acid Tert-Butyl Ester

A solution of [1-(1-benzyl-4-hydroxypyrolidin-3-yl)-2-(tetrahydropyran-2-yloxy)ethyl]-carbamic acid tert-butyl ester (7.8 g, 18.6 mmol, Example 33c) in ethanol (75 mL) was treated, portionwise, with pyridinium-p-toluene sulfonate (5.92 g, 23.6 mmol). The mixture was heated to 85° C. for 18 hours and the solvent removed in vacuo. The residue was then purified by column chromatography on silica gel (10% methanol in ethyl acetate) to obtain 7.5 g of tosylate salt. This solid was suspended in chloroform, treated with potassium carbonate (2.6 g) and stirred at room temperature for 1 hour. The organic layer was concentrated, filtered through Celite and evaporated in vacuo to dryness to obtain 4.3 g of the title compound. ¹H NMR (200 MHz, CDCl₃) δ 7.36-7.28 (m, 6H), 5.68-5.56 (d,1H), 4.42-4.28 (bs, 1H), 3.98-3.58 (m, 6H), 2.82-2.40 (m, 5H), 1.43 (s, 9H).

e) (5-Benzylhexahydrofuro[2,3-c]pyrrol-3-yl)carbamic Acid Tert-Butyl Ester

A −70° C. suspension of [1-(1-benzyl-4-hydroxypyrrolidin-3-yl)-2-hydroxyethyl]-carbamic acid tert-butyl ester (4.35 g, 13.0 mmol, Example 33d) in dichloromethane was treated, dropwise, with (diethylamino)sulfur trifluoride (DAST) (1.7 mL, 12.90 mmol). After stirring at −70° C. for half an hour, the solution was allowed to warm to 15° C. The solvent was removed in vacuo and the residue purified by column chromatography on silica gel eluting with a mixture of ethyl acetate:hexanes (1:2) to obtain 1.9 g of the title compound. ¹H NMR (200 MHz, CDCl₃) δ 7.32-7.26 (m, 5H), 4.82-4.70 (m, 1H), 4.68-4.58 (t, 1H), 4.20-4.10 (m, 1H), 4.04-3.92 (m, 1H), 3.72-3.60 (m, 1H), 3.50 (s, 2H), 2.92-2.70 (m, 2H), 2.65-2.55 (m, 1H), 2.45-2.26 (m, 2H), 1.43 (s, 9H).

f) (Hexahydrofuro[2,3-c]pyrrol-3-yl)carbamic Acid Tert-Butyl Ester

A suspension of (5-benzylhexahydrofuro[2,3-c]pyrrol-3-yl)carbamic acid tert-butyl ester (1.7 g, 5.3 mmol, Example 33e), ammonium formate (1.9 g, 30 mmol) and 10% palladium-carbon (1.7 g) in dry methanol (20 mL) was heated at 70° C. for 1 hour. The cooled mixture was then filtered through Celite and the solvent removed in vacuo affording 1.2 g of the title compound. ¹H NMR (200 MHz, CDCl₃) δ 4.84-4.75 (m, 1H), 4.68-4.60 (t, 1H), 4.08-3.98 (m, 1H), 3.96-3.85 (m, 1H), 3.60-3.46 (m, 1H), 3.10 (d, 1H), 2.96 (d, 2H), 2.78-2.68 (dd, 1H), 2.60-2.48 (m, 1H), 1.85-1.75 (m, 1H), 1.45 (s, 9H).

g) [5-(1-Cyclopropyl-6-fluoro-8-methyldioxo-1,2,3,4-tetrahydroquinazolin-7-yl)hexahydrofuro[2,3-c]pyrrol-3-yl]carbamic Acid Tert-Butyl Ester

A solution of 1-cyclopropyl-6,7-difluoro-8-methyl-1H-quinazolinedione (0.55 g, 2.19 mmol), (hexahydrofuro[2,3-c]pyrrol-3-yl)carbamic tert-butyl ester (1.0 g, 4.38 mmol, Example 33f) and triethylamine (1.2 mL, 8.53 mmol) in dimethylsulfoxide (2 mL) was heated at 110° C. for 4 days and 120° C. for 3 days. The cooled mixture was diluted with water (50 mL) and extracted with ethyl acetate (2×100 mL). The combined extracts were washed with water (2×100 mL), brine (1×100 mL), dried with sodium sulfate and concentrated in vacuo. The residue was purified by column chromatography on silica gel eluting with ethyl acetate:hexanes (1:1) yielding 0.29 g of the title compound. ¹H NMR (200 MHz, CDCl₃) δ 8.2 (s, 1H), 7.57 (d, 1H), 4.92-4.75 (m, 2H), 4.25-4.08 (m, 2H), 3.78-3.28 (m, 5H), 2.90-2.76 (m, 1H), 2.50 (s, 3H), 1.70-1.58 (m, 1H), 1.46 (s, 9H), 1.18-1.08 (m, 2H), 0.68-0.56 (m, 2H).

h) 7-(3-Aminohexahydrofuro[2,3-c]pyrrol-5-yl)-1-cyclopropyl-6-fluoro-8-methyl-1H-quinazolinedione Hydrochloride

Hydrogen chloride gas was bubbled through a 5° C. solution of [5-(1-cyclopropyl-6-fluoro-8-methyldioxo-],2,3,4-tetrahydroquinazolin-7-yl)hexahydrofuro[2,3-c]pyrrol-3-yl]carbamic acid, tert-butyl ester (0.29 g, 0.63 mmol, Example 33g) in a mixture of dichloromethane (6 mL) and diethyl ether (40 mL) for 15 minutes. After stirring at 10-15° C. for 1 hour, the solid was collected by filtration and washed with diethyl ether to obtain 0.17 g of the title compound. ¹H NMR (200 MHz, DMSO-d₆) δ 8.28-8.18 (m, 2H), 7.42 (d, 1H), 4.85-4.75 (m, 1H), 4.16-4.02 (m, 1H), 3.85-3.70 (m, 2H), 3.58-3.25 (m, 6H), 3.02-2.88 (m, 1H), 2.43 (s, 3H), 1.10-0.98 (m, 2H), 0.58-0.45 (m, 2H). MSCI: m/z 361 (MH⁺).

### EXAMPLE 34

a) {1-[1-(1-Cyclopropyl-6-fluoro-8-methyldioxo-1,2,3,4-tetrahydroquinazolin-7-yl)-4,4-dimethylpyrrolidin-3-yl]ethyl}carbamic Acid Tert-Butyl Ester

A solution of 1-cyclopropyl-6,7-difluoro-8-methyl-1H-quinazolinedione (0.44 g, 1.74 mmol), [1-(4,4-dimethypyrrolidin-3-yl)ethyl]carbamic acid tert-butyl ester (0.85 g, 3.51 mmol, [PCT Int. applic. WO 0153273 A1) and triethylamine (0.98 mL, 6.98 mmol) in dimethylsulfoxide (2 mL) was heated in a sealed tube at 110° C. for 40 hours. The cooled reaction was diluted with water (50 mL) and extracted with ethyl acetate (2×100 mL). The combined extracts were washed with water (2×100 mL), brine (100 mL), dried with sodium sulfate and concentrated in vacuo. The residue was then purified by column chromatography on silica gel eluting with ethyl acetate:hexanes (1:2) to give 0.4 g of the title compound. ¹H NMR (200 MHz, CDCl₃) δ 8.12-8.02 (bs, 1H), 7.50 (d, 1H), 4.60-4.48 (m, 1H), 4.00-3.82 (m, 1H), 3.80-3.66 (t, 1H), 3.58-3.45 (m, 2H), 3.38-3.26 (m, 1H), 3.15 (d, 1H), 2.37 (s, 3H), 2.00-1.82 (m, 1H), 1.43 (s, 9H), 1.25 (d, 3H), 1.17 (d, 6H), 1.30-1.10 (m, 2H), 0.74-0.56 (m, 2H).

b) 7-[4-(Aminoethyl)-3,3-dimethylyrrolidin-1-yl]-1-cylcopropyl-6-fluoro-8-methyl-1H-quinazolinedione Hydrochloride

Hydrogen chloride gas was bubbled through a 5° C. solution of {1-[1-(1-cyclopropyl-6-fluoro-8-methyldioxo-1,2,3,4-tetrahydroquinazolin-7-yl)-4,4-dimethylpyrrolidin-3-yl]ethyl}carbamic acid tert-butyl ester (0.48 g, 1 mmol, Example 34a) in a mixture of dichloromethane (20 mL) and ether (50 mL) for 15 minutes. After stirring for 1 hour, the resulting solid was removed by filtration, washed with ether and dried to obtain 0.37 g of the title compound. ¹H NMR (200 MHz, DMSO-d₆) δ 8.25-8.10 (bs, 2H), 7.35 (d, 1H), 3.82-3.56 (m, 2H), 3.46-3.22 (m, 4H), 3.05 (d, 1H), 2.38 (s, 3H), 2.22-2.02 (m, 1H), 1.36 (d, 3H), 1.12 (d, 6H), 1.20-1.00 (m, 2H), 0.62-0.38 (m, 2H). MSCI: m/z 375 (MH⁺).

### EXAMPLE 35

a) [2-(1-Cyclopropyl-6-fluorQ-8-methyldioxo-1,2,3,4-tetrahydroquinazolin-7-yl)octahydroisoindol-4-yl]carbamic Acid Tert-Butyl Ester

A mixture of 1-cyclopropyl-6,7-difluoro-8-methyl-1H-quinazolinedione (0.40 g, 1.50 mmol), (octahydroisoindol-4-yl)carbamic acid tert-butyl ester (1.1 g, 4.50 mmol, [Patent applic. WO 96/9637495]) and 1,1,3,3-tetramethylguanidine (0.56 mL, 4.5 mmol) in dimethyl sulfoxide (1.5 mL) was heated at 80° C. for four days. The mixture was cooled, diluted with water and extracted with ethyl acetate. The combined organic extracts were dried over sodium sulfate, filtered and concentrated under vacuum. The residue was purified by column chromatography (1:1 hexane/ethyl acetate, 0.5% triethylamine) to afford the title compound (0.217 g). ¹H NMR (400 MHz, CDCl₃): δ 8.23 (bs; 1H), 7.56 (d, 1H), 4.45-4.42 (m, 1H), 3.95-3.81 (m, 3H), 3.37-3.33 (m, 1H), 3.22-3.18 (m, 1H), 2.95-2.83 (m, 2H), 2.34 (s, 3H), 2.18-2.11 (m, 2H), 1.82-1.78 (m, 2H), 1.62-1.55 (m, 2H), 1.35 (s, 9H), 1.01-0.94 (m, 3H), 0.71-0.55 (m, 2H). MSCI: m/z 473 (MH⁺).

b) 7-(4-Aminooctahydroisoindol-2-yl)-1-cyclopropyl-6-fluoro-8-methyl-1H-quinazolinedione Hydrochloride

[2-(1-Cyclopropyl-6-fluoro-8-methyldioxo-1,2,3,4-tetrahydroquinazolin-7-yl)octahydroisoindol-4-yl]carbamic acid tert-butyl ester (0.217 g, 0.46 mmol, Example 35a) was dissolved in ether (20 mL), cooled in an ice bath, and hydrogen chloride gas was bubbled through the solution for 15 minutes. The mixture was stirred at 5° C. for four hours then filtered to provide 0.147 g of the title compound as a solid. ¹H NMR (400 MHz, DMSO): δ 8.24 (bs, 3H), 7.35 (d, 1H), 3.95-3.90 (m, 2H), 3.44-3.41 (m, 2H), 3.31-3.28 (m, 1H), 3.03-2.89 (m, 2H), 2.41-2.22 (m, 4H), 1.78-1.61 (m, 2H), 1.59-1.55 (m, 3H), 1.37-1.04 (m, 4H), 0.60-0.56 (m, 1H), 0.47-0.44 (m, 1H). MSCI: m/z 373 (MH⁺).

### EXAMPLE 36

a) 2,4,5-Trifluoro-3-hydroxybenzoic Acid Methyl Ester

To a solution of 2,4,5-trifluoro-3-hydroxybenzoic acid (10.86 g, 56.56 mmol) in methanol (100 mL) was added concentrated sulfuric acid (1.50 mL). The reaction mixture was heated at reflux for 5 hours and the solvent removed in vacuo. The residue was dissolved in dichloromethane (600 mL), washed with brine (3×500 mL), dried over sodium sulfate, and concentrated under reduced pressure to give the title compound as white crystals (10.75 g). ¹H NMR (400 MHz, DMSO-d₆) δ 11.40 (s, 1H), 7.38 (m, 1H), 3.82 (s, 3H).

b) 3-tert-Butoxycarbonylmethoxy-2,4,5-trifluorobenzoic Acid Methyl Ester

A 0° C. solution of 2,4,5-trifluoro-3-hydroxybenzoic acid methyl ester (9.82 g, 47.67 mmol, Example 36a) in N,N-dimethylformamide (120 mL) was treated portionwise with sodium hydride (2.30 g, 57.2 mmol, 60% in mineral oil). After stirring at 0° C. for 20 min., tert-butyl bromoacetate (7.90 mL, 52.4 mmol) was added, and the mixture stirred at room temperature for 18 hours. The reaction mixture was adjusted to pH 8.0 by the addition of saturated ammonium chloride and extracted with dichloromethane (800 mL). The organic layer was washed with brine (3×600 mL), dried over sodium sulfate and the solvent removed in vacuo. The residue was purified by flash chromatography (dichloromethane) to yield the title compound (15.0 g). ¹H NMR (400 MHz, CDCl₃) δ 7.50 (m, 1H), 4.72 (s, 2H), 3.92 (s, 3H), 1.44 (s, 9H).

c) 3-Carboxymethoxy-2,4,5-trifluorobenzoic Acid Methyl Ester

To a solution of 3-tert-butoxycarbonylmethoxy-2,4,5-trifluorobenzoic acid methyl ester (]5.00 g, Example 36b) in dichloromethane (100 mL) was added trifluoroacetic acid (50 mL) and the mixture was stirred at room temperature for 4 hours. The mixture was concentrated in vacuo and the residue crystallized (hexane/dichloromethane) to afford the title compound as white crystals (10.95 g).

¹H NMR (400 MHz, DMSO-d₆) δ 13.28 (bs, 1H), 7.62 (m, 1H), 4.90 (s, 2H), 3.86 (s, 3H).

d) 2,4,5-Trifluoro-3-fluoromethoxybenzoic Acid Methyl Ester

To a solution of 3-carboxymethoxy-2,4,5-trifluorobenzoic acid methyl ester (2.49 g, 9.43 mmol, Example 36c) in dichloromethane (60 mL) was added xenon difluoride (2.38 g, 14.1 mmol) and the mixture was stirred at room temperature for 18 hours. The reaction mixture was washed with saturated aqueous sodium bicarbonate (2×50 mL), brine (2×50 mL), dried with sodium sulfate and concentrated in vacuo. The residue was purified by flash chromatography (1:1 dichloromethane/hexanes) to give the title compound (1.00 g). ¹H NMR (400 MHz, CDCl₃) δ 7.64 (m, 1H), 5.68 (d, 2H), 3.98 (s, 3H).

e) 2,4,5-Trifluoro-3-fluoromethoxybenzamide

To a solution of 2,4,5-trifluoro-3-fluoromethoxybenzoic acid methyl ester (1.00 g, 4.20 mmol, Example 36d) in methanol (5 mL) was added aqueous ammonia (25 mL). The mixture was stirred at room temperature for 18 hours and extracted with dichloromethane (3×20 mL). The combined organic layers were dried over sodium sulfate, concentrated in vacuo and the residue purified by chromatography (dichloromethane to 95:5 dichloromethane/methanol gradient) to give the title compound (0.65 g). ¹H NMR (400 MHz, CDCl₃) δ 7.84 (m, 1H), 6.60 (bs, 1H), 5.90 (bs, 1H), 5.68 (d, 2H).

f) 1-Cyclopropyl-3-(2,4,5-trifluoro-3-fluoromethoxybenzoyl)urea

A solution of 2,4,5-trifluoro-3-fluoromethoxybenzamide (6.65 g, 2.91 mmol, Example 36e) in 1,2-dichloroethane (12 mL) was treated dropwise with oxalyl chloride (0.78 mL, 8.73 mmol). The mixture was stirred at room temperature for 1 hour, heated at reflux for 4 hours, and the solvent was removed under reduced pressure to give 2,4,5-trifluoro-3-fluoromethoxybenzoyl isocyanate, which was dissolved in dioxane (10 mL) and treated with a solution of cyclopropylamine (0.62 mL, 8.73 mmol) in dioxane (2 mL). The mixture was warmed to room temperature for 18 hours and concentrated in vacuo. The residue was dissolved in ethyl acetate, washed with brine, dried over sodium sulfate and evaporated. This residue was purified by chromatography (9:1 methylene chloride/methanol) to give the title compound (0.88 g). ¹H NMR (400 MHz, CDCl₃) δ 8.62 (d, 1H), 8.40 (bs, 1H), 7.70 (m, 1H), 5.69 (d, 2H), 2.78 (m, 1H), 0.82 (m, 1H), 0.62 (m, 1H).

g) 1-Cyclopropyl-6,7-difluoro-8-fluoromethoxy-1H-quinazolinedione

A solution of 1-cyclopropyl-3-(2,4,5-trifluoro-3-fluoromethoxybenzoyl)urea (0.88 g, 2.90 mmol, Example 36f) in tetrahydrofuran (35 mL) was treated portionwise with sodium hydride (0.35 g, 8.82 mmol, 60% in mineral oil). The mixture was stirred at room temperature for 30 min. and then heated at reflux overnight. The cooled reaction mixture was adjusted to pH 8.0 by the addition of a saturated solution of ammonium chloride and extracted with dichloromethane (3×50 mL). The combined organic layers were washed with water, dried with sodium sulfate and concentrated in vacuo. The residue was purified by chromatography (9:1 methylene chloride/methanol) to give the title compound (0.56 g). ¹H NMR (400 MHz, CDCl₃) δ 8.20 (bs, 1H), 7.82 (t, 1H), 5.64 (d, 2H), 3.38 (m, 1H), 1.20 (m, 2H), 0.78 (m, 2H).

h) {(S)-1-[(R)-1-(1-Cyclopropyl-6-fluoro-8-fluoromethoxydioxo-1,2,3,4-tetrahydroquinazolin-7-yl)pyrrolidin-3-yl]ethyl}carbamic Acid Tert-Butyl Ester

A mixture of 1-cyclopropyl-6,7-difluoro-8-fluoromethoxy-1H-quinazolinedione (0.160 g, 0.56 mmol, Example 36g), ((S)-(R)-1-pyrrolidin-3-ylethyl)carbamic acid tert-butyl ester (0.240 g, 1.12 mmol), triethylamine (0.23 mL, 1.68 mmol) and dimethyl sulfoxide (4 mL) was heated at 90° C. for 2 hours. The cooled reaction mixture was diluted with ethyl acetate (20 mL) and washed with brine (3×20 mL). The combined organic layers were dried over sodium sulfate and concentrated, and the residue was purified by flash chromatography (9:1 methylene chloride/methanol) to give the title compound (0.29 g). ¹H NMR (400 MHz, CDCl₃) δ 8.00 (bs, 1H), 7.56 (d, 1H), 5.50-5.20 (m, 2H), 4.50 (d, 1H), 3.80-3.50 (m, 5H), 3.25 (m, 1H), 2.20 (m, 1H), 2.05 (m, 1H), 1.62 (m, 1H), 1.43 (s, 9H), 1.25 (d, 3H), 1.20-1.00 (m, 2H), 0.70-0.60 (m, 2H).

i) 7-[(R)-3-((S)-1-Aminoethyl)pyrrolidin-1-yl]-1-cyclopropyl-6-fluoro-8-fluoromethoxy-1H-quinazolinedione Hydrochloride

A 0° C. solution of {(S)-1-[(R)-1-(1-cyclopropyl-6-fluoro-8-fluoromethoxydioxo-1,2,3,4-tetrahydroquinazolin-7-yl)pyrrolidin-3-yl]ethyl}carbamic acid tert-butyl ester (0.286 g, Example 36h) in diethyl ether was saturated with hydrogen chloride gas. The resulting mixture was stirred at room temperature for 2 hours and the solvent removed in vacuo to give the title compound (0.250 g). ¹H NMR (400 MHz, DMSO-d₆) δ 11.30 (s, 1H), 8.08 (bs, 3H), 7.40 (d, 1H), 5.60-5.40 (m, 2H), 3.70-3.40 (m, 4H), 3.25 (m, 1H), 3.10 (m, 1H), 2.30 (m, 1H), 2.06 (m, 1H), 1.62 (m, 1H), 1.25 (d, 3H), 1.04 (m, 1H), 0.92 (m, 1H), 0.70 (m, 1H), 0.60 (m, 1H).

### EXAMPLE 37

a) 3-Difluoromethyl-2,4,5-trifluorobenzoic Acid

Under a nitrogen atmosphere, a −30° C. solution of hexamethyldisilazane (5.5 g, 34 mmol) in anhydrous tetrahydrofuran (30 mL) was treated with n-butyllithium (17.2 mL of 2.5 M hexane solution, 34 mmol). After 30 min at −30° C., the mixture was cooled to −50° C. and a solution of 2,4,5-trifluorobenzoic acid (3.0 g, 17 mmol) in tetrahydrofuran (20 mL) was added by syringe, and the mixture was stirred at −10° C. for 2 hours. The mixture was cooled to −30° C., anhydrous N,N-dimethylformamide (3.8 mL, 38 mmol) was added, and the mixture was allowed to warm to 0° C. for 1 hour. Saturated aqueous ammonium chloride solution was added and mixture acidified with 2 N hydrochloric acid and extracted with ethyl acetate. The organic extracts were combined and washed with water, brine, dried with sodium sulfate and concentrated under vacuum to give 3.47 g of 3-formyl-2,4,5-trifluorobenzoic acid, which was used for the next step without further purification. A room temperature solution of this intermediate in dichloromethane was treated with (diethylamino)sulfur trifluoride (DAST) (13.7 g, 85 mmol), and the reaction mixture was stirred at room temperature for 24 hours. After cooling to 5° C., the reaction was quenched with ice (exothermic reaction!), washed with water and treated with aqueous ammonia for 1 hour at room temperature. The aqueous layer was separated, acidified with 2 N hydrochloric acid and extracted with dichloromethane. The organic extracts were dried over sodium sulfate, concentrated under vacuum and extracted with hot hexanes. The hexane layers were combined and concentrated in vacuo and the residue recrystallized from hexanes/ethyl acetate (10:1) to provide 0.98 g of the title compound as colorless crystals. ¹H NMR (400 MHz, CDCl₃) δ 11.8-10.0 (bs, 1H), 8.02 (m, 1H), 6.98 (t, 1H).

b) 1-Cyclopropyl-3-(3-difluoromethyl-2,4,5-trifluorobenzoyl)urea

To a solution of 3-difluoromethyl-2,4,5-trifluorobenzoic acid (0.46 g, 2 mmol, Example 37a) in anhydrous dichloromethane (10 mL) was added oxalyl chloride (0.63 g, 5 mmol) followed by N,N-dimethylformamide (1 drop). The mixture was stirred for 2 hours and the solvent removed in vacuo. The residue was dissolved in anhydrous benzene and cyclopropylurea (0.4 g, 4 mmol)was added. The mixture was refluxed for 3 hours and diluted with ethyl acetate, washed with water and brine, dried with sodium sulfate and concentrated. Purification by flash chromatography on silica gel (hexane/ethyl acetate 3:1) provided 0.33 g of the title compound as a colorless solid. ¹H NMR (400 MHz, CDCl₃) δ 9.00 (d, 1H), 8.38 (bs, 1H), 7.96 (m, 1H), 6.94 (t, 1H), 2.73 (m, 1H), 0.82 (m, 2H), 0.62 (m, 2H).

c) 1-Cyclopropyl-8-difluoromethyl-6,7-difluoro-1H-quinazolinedione

Under a nitrogen atmosphere, a 0-5° C. solution of 1-cyclopropyl-3-(3-difluoromethyl-2,4,5-trifluorobenzoyl)urea (0.33 g, 1.1 mmol, Example 37b) in anhydrous tetrahydrofuran (10 mL) and N,N-dimethylformamide (0.5 mL) was treated portionwise with sodium hydride (0.16 g of 60% oil dispersion, 3.9 mmol). The mixture was stirred at room temperature for 30 min and at 60° C. for 3 hours. After cooling, the mixture was quenched with ice, quenched with 2 N hydrochloric acid and extracted with ethyl acetate. The organic extracts were washed with water, dried with sodium sulfate and concentrated in vacuo affording 0.38 g of the title compound as a pale yellow solid. ¹H NMR (400 MHz, CDCl₃) δ 8.60 (bs, 1H), 8.06 (m, 1H), 7.40 (t, 1H), 3.31 (bs, 1H), 1.20 (m, 2H), 0.71 (m, 2H).

d) {(S)-1-[(R)-1-(1-Cyclopropyl-8-difluoromethyl-6-fluorodioxo-1,2,3,4-tetrahydroquinazolin-7-yl)pyrrolidin-3-yl]ethyl}carbamic Acid Tert-Butyl Ester

A solution of 1-cyclopropyl-8-difluoromethyl-6,7-difluoro-1H-quinazolinedione (0.29 g, 1.0 mmol, Example 37c), ((S)-(R)-1-pyrrolidin-3-ylethyl)carbamic acid tert-butyl ester (0.43 g, 2 mmol), triethylamine (0.3 g, 3 mmol) and anhydrous dimethylsulfoxide (2 mL) was stirred at 80° C. for 4 hours. The cooled reaction was diluted with water and then extracted with ethyl acetate. The organic layers were combined and washed with water, brine, dried with sodium sulfate and concentrated in vacuo. Purification of the residue by flash chromatography on silica gel (dichloromethane/diethyl ether 2:1) gave 0.42 g of the title compound as a colorless solid. ¹H NMR (400 MHz, CDCl₃) δ 8.34 (bs, 1H), 7.71 (d, 1H), 6.76 (t, 1H), 4.50-4.40 (m, 1H), 3.80-3.33 (m, 6H), 2.34-2.22 (m, 1H), 2.12-2.06 (m, 1H), 1.80-1.68 (m, 1H), 1.43 (s, 9H), 1.28-1.12 (m, 5H), 0.66-0.52 (m, 2H).

e) 7-[(R)-3-((S)-1-Aminoethyl)pyrrolidin-1-yl]-1-cyclopropyl-8-difluoromethyl-6-fluoro-1H-quinazolinedione Hydrochloride

A 0° C. solution of {(S)-1-[(R)-1-(1-cyclopropyl-8-difluoromethyl-6-fluorodioxo-1,2,3,4-tetrahydroquinazolin-7-yl)pyrrolidin-3-yl]ethyl) carbamic acid tert-butyl ester (0.42 g, 0.87 mmol, Example 37d) in dichloromethane (20 mL) was treated with a stream of gaseous hydrogen chloride for 40 minutes. The resulting precipitate was removed by filtration, washed with dichloromethane and dried in vacuo to give 0.32 g of the title compound as colorless crystals. ¹H NMR (400 MHz, DMSO-d₆) δ 11.44 (s, 1H), 8.09 (bs, 3H), 7.58 (d, 1H), 7.18 (t, 1H), 3.64-3.35 (m, 4H), 3.28-3.11 (m, 2H), 2.43-2.30 (m, 1H), 2.12-1.98 (m, 1H), 1.76-1.64 (m, 1H), 1.24 (d, 3H), 1.16-0.97 (m, 2H), 0.61-0.43 (m, 2H). MSCI: m/z 381 (M+).

### EXAMPLE 38

a) 1-Thiophen-2-ylcyclopropanecarbonitrile

A stirred mixture of 2-thiopheneacetonitrile (2.5 g, 20 mmol), benzyltriethylammonium bromide (0.54 g, 2.0 mmol), dichloromethane (20 mL), and 50% aqueous sodium hydroxide solution (8 g, 200 mmol) was cooled to 0° C. and treated dropwise with 1,2-dibromoethane (2.07 mL, 24 mmol). The mixture was allowed to warm to room temperature and stir for two days. After diluting with dichloromethane (20 mL) and water (30 mL), the aqueous layer was extracted with dichloromethane (3×40 mL) and the combined organics were washed with water (30 mL), brine (30 mL), dried with sodium sulfate and concentrated in vacuo. The dark residual oil was purified by flash chromatography (10/90 ethyl acetate/hexane) to give the title compound as a light brown oil (1.40 g ). ¹H NMR (200 MHz, CDCl₃) δ 7.40 (dd, 1H), 7.20 (dd, 1H), 6.95 (dd, 1H), 1.55 (m, 2H), 1.45 (m, 2H).

b) 1-Thiophen-2-ylcyclopropanecarboxylic Acid

A 5° C. solution of 1-thiophen-2-ylcyclopropanecarbonitrile (12.3 g, 82.6 mmol, Example 38a) in ethanol (150 mL) was treated with 6 N sodium hydroxide (100 mL) and stirred at 0° C. for 5 minutes. The reaction mixture was then heated at reflux for 4 hours, the mixture was cooled and the ethanol was removed in vacuo. The basic, aqueous residue was acidified with 6 N hydrochloric acid and extracted with ethyl acetate (3×100 mL). The combined organics were dried over sodium sulfate and the solvent was removed in vacuo to give the title compound as white crystals (13.60 g). ¹H NMR (400 MHz, DMSO-d₆) δ 12.5 (bs, 1H), 7.40 (dd, 1H), 7.00-6.90 (m, 2H), 1.60 (m, 2H), 1.28 (m, 2H).

c) (1-Thiophen-2-ylcyclopropyl)carbamic Acid Tert-Butyl Ester

A room temperature solution of 1-thiophen-2-ylcyclopropanecarboxylic acid (3.20 g, 19.0 mmol, Example 38b) in tert-butanol (50 mL) was treated dropwise with diphenylphosphoryl azide (5.48 mL, 24.8 mmol) followed by triethylamine (4.23 mL, 30.4 mmol). The mixture was stirred at room temperature for 2 hours and then heated at reflux for 20 hours. The solvent was removed in vacuo and the residue chromatographed on silica gel (10/90 ethyl acetate/hexane) to yield the title compound (3.20 g). ¹H NMR (200 MHz, CDCl₃) δ 7.10 (dd, 1H), 6.90 (dd, 1H), 6.80 (dd, 1H), 5.35 (bs, 1H), 1.45 (s, 9H), 1.30 (m, 2H), 1.22 (m, 2H).

d) [1-(5-Tributylstannylthiophen-2-yl)cyclopropyl]carbamic Acid Tert-Butyl Ester

A −78° C. solution of compound (1-thiophen-2-ylcyclopropyl)carbamic acid tert-butyl ester (1.24 g, 5.00 mmol, Example 38c) in tetrahydrofuran (30 mL) was treated dropwise with n-butyllithium (2.5 M in hexanes, 5 mL, 12.5 mmol), warmed to −20° C. and stirred for 3 hours. After recooling to −78° C., a solution of tri-n-butyltin chloride (1.9 g, 6.0 mmol) in tetrahydrofuran (8 mL) was added dropwise. The reaction mixture was warmed to room temperature, and partitioned between ethyl acetate and water. The aqueous layer was extracted with ethyl acetate, and the combined extracts washed with water, brine, dried with sodium sulfate, filtered, and concentrated. The residue was purified by silica gel chromatography (4/96 ethyl acetate/hexane and 0.5% triethylamine) to provide the title compound (1.08 g) as a colorless oil. ¹H NMR (400 MHz, CDCl₃) δ 6.94 (d, 1H), 6.92 (d, 1H), 5.40 (bs, 1H), 1.55 (m, 6H), 1.45 (s, 9H), 1.35 (m, 6H), 1.26 (m, 4H), 1.07 (m, 6H), 0.90 (t, 9H).

e) {1-[5-(1-cyclopropyl-6-fluoro-8-methyldioxo-1,2,3,4-tetrahydroquinazolin-7-yl)thiophen-2-yl]cyclopropyl}carbamic Acid Tert-Butyl Ester

A mixture of [1-(5-tri-n-butylstannylthiophen-2-yl)cyclopropyl]carbamic acid tert-butyl ester (0.74 g, 1.4 mmol, Example 38d), 1-cyclopropyl-6-fluoro-7-iodo-8-methyl-1H-quinazolinedione (0.50 g, 1.4 mmol, Example 25h), dichlorobis(triphenylphosphine)palladium(II) (0.11 g, 0.16 mmol), and triphenylarsine (0.165 g, 0.54 mmol) in toluene (20 mL) was heated at 90-95° C. for 24 hours. After evaporation of the solvent, the residue was purified by chromatography (1:1 ethyl acetate/hexane) to give the title compound (0.40 g). ¹H NMR (400 MHz, CDCl₃) δ 8.38 (bs, 1H), 7.70 (d, 1H), 6.90 (d, 1H), 6.84 (d, 1H), 5.40 (bs, 1H), 3.38 (m, 1H), 2.50 (s, 3H), 1.45 (s, 9H), 1.40-1.20 (m, 6H), 0.78 (m, 2H).

f) 7-[5-(1-Aminocyclopropyl)thiophen-2-yl]-1-cyclopropyl-6-fluoro-8-methyl-1H-quinazolinedione Hydrochloride

A 0° C. solution of {1-[5-(1-cyclopropyl-6-fluoro-8-methyldioxo-1,2,3,4-tetrahydro-quinazolin-7-yl)thiophen-2-yl]cyclopropyl} carbamic acid tert-butyl ester (0.209 g, Example 38e) in dichloromethane was saturated with hydrogen chloride gas. After stirring at room temperature overnight, the resulting precipitate was removed by filtration, washed with dichloromethane and dried in vacuo to give the title compound (0.150 g). ¹H NMR (400 MHz, DMSO-d₆) δ 11.60 (s, 1H), 9.00 (bs, 3H), 7.60 (d, 1H), 7.40 (d, 1H), 7.20 (d, 1H), 3.34 (m, 1H), 2.40 (s, 3H), 1.50 (m, 2H), 1.38 (m, 2H), 1.05 (m, 2H), 0.62 (m, 2H).

### EXAMPLE 39

a) 1-Cyclopropyl-3-(3-difluoromethoxy-2,4,5-trifluorobenzoyl)urea

A solution of 3-difluoromethoxy-2,4,5-trifluorobenzamide (80 g, 330 mmol, EP 352123 A2), oxalyl chloride (126.6 g 990 mmoles) and 1,2-dichloroethane (800 ml) was heated at reflux for 4 hours. The solvent was removed in vacuo and the residue was dissolved in dry 1,4-dioxane (700 ml) and cooled to 5° C. Cyclopropylamine (36.5 g, 640 mmol) was added and the reaction mixture was stirred at room temperature for 16 hours. The solvent was removed in vacuo and the residue was dissolved in ethyl acetate, washed with brine, dried with sodium sulfate and concentrated in vacuo. The residue was triturated with hexanes, and the solid was removed by filtration, washed with hexanes and dried to give 88 g of the title compound as a light yellow semi solid. ¹H NMR (400 MHz, DMSO-d₆): δ 11.00 (s, 1H), 8.2 (s, 1H), 7.8 (m, 1H), 7.25 (t, 1H), 2.63 (s, 1H), 0.65 (m, 2H), 0.50 (m, 2H) b) 1-Cyclopropyl-8-difluoromethoxy-6,7-difluoro-1H-quinazolinedione Under a nitrogen atmosphere, a solution of 1-cyclopropyl-3-(3-difluoromethoxy-2,4,5-trifluorobenzoyl)urea (87 g, 277 mmol, Example 39a) in anhydrous tetrahydrofuran (750 ml) and dimethylformamide (75 ml), was treated portionwise over 45 minutes, with sodium hydride (38 g, 60% in mineral oil, 950 mmol). After heating at reflux for 2 hours, the reaction mixture was cooled to room temperature, diluted with 5° C. water, acidified with 2N hydrochloric acid and extracted with ethyl acetate. The organic extract was washed with a 10% aqueous sodium carbonate, water, brine, dried with sodium sulfate and concentrated in vacuo. Purification of the residue by flash chromatography on silica gel (hexane/ethyl acetate 2:1) afforded 25 g of the title compound as a light yellow solid.

¹H NMR (400 MHz, CDCl₃) δ 9.00 (bs, 1H), 7.95 (dd, 1H), 6.68 (t, 1H), 3.23 (m, 1H), 1.21 (m, 2H), 0.78 (m, 2H).

c) 1-((S)-1-Phenylethyl)pyrrolidine-3-carboxylic Acid Dibenzylamide

N,N-Dibenzylacrylamide (79.5 g, 0.317 mol, [WO 9801417]) and N-(methoxymethyl)-N-(trimethylsilylmethyl)-(S)-α-methylbenzylamine (103 g, 412 mmol) were dissolved in dichloromethane (1500 mL) and cooled to ⁰° C. Trifluoroacetic acid (1 M in dichloromethane, 27 mL) was added over a period of 20 minutes and the resulting reaction mixture was stirred at room temperature overnight. The mixture was washed with aqueous sodium bicarbonate, brine, dried over sodium sulfate and concentrated. The residue was purified by flash chromatography (10:2:0.1 heptane/ethyl acetate/triethylamine) to afford the title compound (97.7 g), which was used in the following reaction without further purification.

d) Dibenzyl-{1-[(R)-1-((S)-1-phenylethyl)pyrrolidin-3-yl]cyclopropyl}amine

Ethylmagnesium bromide (3M in ether, 178 mL) was added to dry tetrahydrofuran (1400 mL) and the solution was cooled to −78° C. under a nitrogen atmosphere. A solution of titanium tetraisopropoxide (66.0 mL, 0.228 mol) in dry tetrahydrofuran (150 mL) was then added while maintaining the temperature below −68° C. After the addition was complete, the solution was stirred for three minutes and then 1-((S)-1-phenylethyl)pyrrolidine-3-carboxylic acid dibenzylamide (86.6 g, 0.218 mmol, Example 39c) dissolved in dry tetrahydrofuran (150 mL) was added, maintaining the temperature below −68° C. The reaction mixture was allowed to warm to room temperature, stirred for 1 hour, then heated at reflux for 1 hour. The reaction mixture was then cooled to 8 ° C., and ethylmagnesium bromide (3M in ether, 150 mL) was added followed by the rapid addition of titanium tetraisopropoxide (55.6 mL, 192 mmol) in tetrahydrofuran (150 mL). The resulting mixture was stirred at room temperature for 1 hour before being quenched with aqueous ammonium chloride (3000 mL) and water (800 mL). The mixture was filtered through Celite, rinsed with ether and the organic layer separated. The mixture was made basic (pH 8.5) with sodium hydroxide and extracted with ether. The combined organic layers were combined and dried over sodium sulfate, concentrated and purified by flash chromatography (10:1:0.1 heptane/ethyl acetate/triethylamine) to provide the title compound (31.3 g) as colorless crystals: mp 76-76.5° C.

e) (R)-1-pyrrolidin-3-yl-cyclopropylamine

20% palladium on carbon (0.25 g) was added to a solution of dibenzyl-{1-[(R)-1-((S)-1-phenylethyl)pyrrolidin-3-yl]cyclopropyl}amine (1.0 g, 2.4 mmol, Example 39d) in glacial acetic acid (50 mL) and the reaction vessel pressurized with hydrogen gas (48 psi) overnight. The mixture was then filtered, concentrated in vacuo and the residual dissolved in methanol (20 mL) and stirred with IRA-400-OH basic ion exchange resin. The mixture was filtered after 1 hour and the filtrate concentrated to give the title compound (0.307 g): ¹H NMR (CD₃OD) δ 3.46-3.37 (m, 2H), 3.24 (m, 1H), 3.13 (dd, 1H), 2.25-2.09 (m, 2H), 1.88 (m, 2H), 0.73-0.59 (m, 4H).

f) 7-[(R)-3-(1-Aminocyclopropyl)pyrrolidin-1-yl]-1-cyclopropyl-8-diflouromethoxy-6-fluoro-1H-quinazolinedione

1-Cyclopropyl-8-difluoromethoxy-6,7-difluoro-1H-quinazolinedione (0.49 g, 1.62 mmol, Example 39b) and (R)-1-pyrrolidin-3-ylcyclopropylamine (0.31 g, 2.4 mmol, Example 39e) in dimethyl sulfoxide (5 mL) were heated at 90° C. for 5 hours. The solution was diluted with brine and extracted with ethyl acetate. The organic layers were then combined, dried over magnesium sulfate, filtered and concentrated. The residue was purified by flash silica gel chromatography (2-5% methanol/dichloromethane) to afford the title compound (0.51 g) as a solid, which was re-crystallized to give an analytically pure sample: mp 210-214° C.; ¹H NMR (DMSO-d₆) δ 7.46 (d, 1H), 6.79 (t, 1H), 3.77 (m, 1H), 3.65 (dt, 1H), 3.48 (m, 2H), 3.35 (bs, 1H), 3.12 (m, 1H), 2.05-1.88 (m, 2H), 1.78 (m, 1H), 1.11 (m, 1H), 0.96 (m, 1H), 0.73-0.58 m, 2H), 0.51-0.42 m, 4H).

### EXAMPLE 40

a) {(S)-1-[(R)-1-(1-Cyclopropyl-6-fluoro-8-difluoromethoxydioxo-1,2,3,4-tetrahydroquinazolin-7-yl)pyrrolidin-3-yl]ethyl}carbamic Acid Tert-Butyl Ester

A mixture of 1-cyclopropyl-6,7-difluoro-8-difluoromethoxy-1H-quinazolinedione (0.200 g, 0.66 mmol, Example 39b), ((S)-(R)-1-pyrrolidin-3-ylethyl)carbamic acid tert-butyl ester (0.225 g, 1.97 mmol) and dimethyl sulfoxide (2 mL) was heated at 90° C. for 1.5 hours. The solution was then treated with saturated ammonium chloride, stirred for 1 hour then filtered. The collected solid was washed with water and dried to afford the title compound (0.129 g). MSCI: m/z 497 (M+).

b) 7-[(R)-3-((S)-1-Aminoethyl)pyrrolidin-1-yl]-1-cyclopropyl-6-fluoro-8-difluoromethoxy-1H-quinazolinedione Hydrochloride

A solution of {(S)-1-[(R)-1-(1-Cyclopropyl-6-fluoro-8-difluoromethoxydioxo-1,2,3,4-tetrahydroquinazolin-7-yl)pyrrolidin-3-yl]ethyl}carbamic acid tert-butyl ester (0.129 g, Example 40a) in methanol (3 mL) was treated with a 2 M diethyl ether solution of hydrochloric acid (4 mL, 8 mmol) and allowed to stir for 6 hours. The mixture was then concentrated in vacuo, re-dissolved in water and lyophilized to provide a solid (0.095 g). mp>250° C.; MSCI: m/z 399 (MH⁺).

### EXAMPLE 41

a) 1-Cyclopropyl-6,7-difluoro-5,8-dimethyl-1H-quinazolinedione

A solution of 1-cyclopropyl-6,7-difluoro-8-methyl-1H-quinazolinedione (0.50 g, 2.0 mmol) in tetrahydrofuran (10 mL) was cooled to −20° C. and treated with a 2.0 M tetrahydrofuran solution of lithium diisopropylamine (3.1 mL, 6.3 mmol). The mixture was allowed to stir for 1 hour then cooled to −78° C. and treated with iodomethane (0.31 mL, 5.0 mmol). After stirring for 1 hour, the mixture was poured into saturated ammonium chloride and extracted with ethyl acetate. The extracts were combined, dried with sodium sulfate and purified via silica column chromatography (hexanes/ethyl actetate) to provide a solid (0.206 g). MSCI: m/z 267 (MH⁺).

b) {(S)-1-[(R)-1-(1-Cyclopropyl-6-fluoro-5,8-dimethyldioxo-1,2,3,4-tetrahydro-quinazolin-7-yl)pyrrolidin-3-yl]ethyl}carbamic Acid Tert-Butyl Ester

1-Cyclopropyl-6,7-difluoro-5,8-dimethyl-1H-quinazolinedione (0.20 g, 0.75 mmol, Example 41a), 1,1,3,3-tetramethylguanidine (0.37 mL, 3.0 mmol) and ((S)-(R)-1-pyrrolidin-3-ylethyl)carbamic acid tert-butyl ester (0.68 g, 6.0 mmol) in dimethyl sulfoxide (2 mL) were heated at 90° C. for 2 days. The solution was diluted with saturated ammonium chloride and extracted with ethyl acetate. The organic layers were combined, dried over magnesium sulfate, filtered and concentrated. The residue was purified by flash silica gel chromatography (1:1 hexanes:ethyl acetate) to afford the title compound (0.215 g). MSCI: m/z 461 (MH⁺).

c) 7-[(R)-3-((S)-1-Aminoethyl)pyrrolidin-1-yl]-1-cyclopropyl-6-fluoro-5,8-dimethyl-1H-quinazolinedione Hydrochloride

A solution of {(S)-1-[(R)-1-(1-cyclopropyl-6-fluoro-5,8-dimethyldioxo-1,2,3,4-tetrahydro-quinazolin-7-yl)pyrrolidin-3-yl]ethyl} carbamic acid tert-butyl ester (0.22 g, 0.75 mmol, Example 41b) in a mixture of methanol (3 mL) and dichloromethane (3 mL) was treated with a 2.0 M diethyl ether solution of hydrogen chloride (4 mL, 8 mmol) and allowed to stir for 6 hours. The mixture was concentrated in vacuo, re-dissolved in water and lyophilized to provide a solid (0.095 g); mp 207° C.; MSCI: m/z 461 (MH⁺).

### EXAMPLE 42

a) 2,4-Dibromo-3-difluoromethoxybenzamide

To a suspension of 2,4-dibromo-3-difluoromethoxybenzoic acid (73.3 g, 210 mmol, [WO 9921849 A1]), N,N-dimethylformamide (1.0 ml) and dichloromethane (700 ml) was added dropwise, oxalyl chloride (40.3 g, 320 mmol). After heating at reflux for 5 hours and stirring at room temperature for 2 hours, the solvent was removed in vacuo. The residue was dissolved in anhydrous tetrahydrofuran (500 ml) and added to a −70° C. solution of diethyl ether saturated with ammonia gas. The reaction mixture was stirred at room temperature for 2 hours and evaporated under vacuum. The residue was dissolved in ethyl acetate, washed with water, brine, dried over sodium sulfate, filtered and evaporated in vacuo to give 58.0 g of the title compound as a white solid, mp 186° C. ¹H NMR (400 MHz, CDCl₃): δ 7.65 (d, 1H); 7.39 (d, 1H); 6.62 (t, 1H); 6.0 (bs, 2H).-

b) 1-Cyclopropyl-3-(2,4-dibromo-3-difluoromethoxybenzoyl)urea

To a suspension of 2,4-dibromo-3-difluoromethoxybenzamide (58.0 g, 168 mmol, Example 42a) in 1,2-dichloroethane (600 ml) was added oxalyl chloride (53.0 g, 420 mmol). After stirring for 1 hour at room temperature, the mixture was refluxed for 5 hours, cooled and the solvent removed in vacuo. The oily residue was dissolved in dioxane (500 ml), cooled to 0° C. and treated with a solution of cyclopropylamine (20.0 g, 350 mmol) in dioxane (50 ml). After stirring at room temperature for 18 hours, the solvent was removed in vacuo and the residue triturated with a mixture of diethyl ether and hexanes. The resulting solid was collected by filtration, washed with hexanes and dried in vacuo to give 60.8 g of the title compound as a white solid, mp 170° C. ¹H NMR (400 MHz, CDCl₃) δ 8.45 (s, 1H); 8.25 (s, 1H); 7.65 (d, 1H); 7.25 (d, 1H); 6.65 (t, 1H); 2.65 (m, 1H); 0.80 (m, 2H); 0.62 (m, 2H).

c) 7-Bromo-1-cyclopropyl-8-difluoromethoxy-1H-quinazolinedione

To a 0° C. solution of 1-cyclopropyl-3-(2,4-dibromo-3-difluoromethoxy benzoyl)urea (60.0 g, 140 mmol, Example 42b) in anhydrous tetrahydrofuran (600 ml) was added dropwise, at room temperature, potassium bis(trimethylsilyl)amide (700 ml, 0.5 M in toluene, 0.35 mol). After stirring for 20 minutes, 18-crown-6 (16.8 g) was added and the reaction heated to reflux for 2.5 hours. The solvent was removed in vacuo and the residue partitioned between ethyl acetate and 1N hydrochloric acid. The organic layer was washed with water, brine, dried with magnesium sulfate, filtered and evaporated under vacuum. Purification of the residue by chromatography on silica gel (hexane/ethyl acetate) yielded 13.6 g of the title compound as a white solid, mp 2440 C. ¹H NMR (CDCl₃): δ 8.17 (s, 1H); 7.88 (d, 1H); 7.48 (d, 1H); 6.45 (t, 1H); 3.39 (m, 1H); 1.22 (m, 2H); 0.65 (m, 2H).

d) 1-Cyclopropyl-8-difluoromethoxy-7-((R)-1-methyl-2-trityl-2,3-dihydro-1H-isoindol-5-yl)-1H-quinazolinedione

To a suspension of 2-[(1R)-1-methyl-2-trityl-2,3-dihydro-1H-5-isoindolyl]-1,3,6,2-dioxazaborocane (0.375 g, 0.768 mmol, [EP 1031569]) in ethyl acetate (1.8 mL) and water (0.8 mL) was added 7-bromo-1-cyclopropyl-8-difluoromethoxy-1H-quinazolinedione (0.10 g, 0.288 mmol, Example 42c), sodium carbonate (0.128 g, 1.21 mmol) and bis(triphenylphosphine)palladium(II) chloride (0.040 g, 0.057 mmol). The resulting mixture was heated at 80° C. for 24 hours. The mixture was cooled to room temperature and partitioned between dichloromethane and water. The recovered organics were washed with brine, dried over magnesium sulfate, filtered, and concentrated in vacuo. The resulting residue was purified by flash silica gel chromatography (100:0 to 50:50 hexanes:ethyl acetate) to afford the title compound (0.192 g) as a yellow solid: MSCI: mnz 642 (MH⁺).

e) (R)-5-(1-Cyclopropyl-8-difluoromethoxydioxo-1,2,3,4-tetrahydroquinazolin-7-yl)-1-methyl-1,3-dihydroisoindole-2-carboxylic Acid Tert-Butyl Ester

Hydrogen chloride gas was bubbled into a 0° C. solution of 1-cyclopropyl-8-difluoro-methoxy-7-((R)-1-methyl-2-trityl-2,3-dihydro-1H-isoindol-5-yl)-1H-quinazolinedione (0.192 g, 0.299 mmol, Example 42d) in methanol (5 mL) and dichloromethane (5 mL) for 20 minutes. The reaction mixture was warmed to room temperature and stirred for 24 hours. The solvent was removed in vacuo, and the resulting solid was treated with dichloromethane (10 mL), triethylamine (0.30 mL), and di-tert-butyl dicarbonate (0.323 g). After 24 hours at room temperature, the reaction mixture was concentrated in vacuo and the resulting residue was purified by preparatory thin layer chromatography eluting with 20% ethyl acetate in dichloromethane to provide the title compound (0.033 g, 25%) as a yellow solid: MSCI: m/z 500 (MH⁺).

f) 1-Cyclopropyl-8-difluoromethoxy-7-((R)-1-methyl-2,3-dihydro-1H-isoindol-5-yl)-1H-quinazolinedione Hydrochloride

Hydrogen chloride gas was bubbled into a 0° C. solution of (R)-5-(1-cyclopropyl-8-difluoromethoxydioxo-1,2,3,4-tetrahydroquinazolin-7-yl)-1-methyl-1,3-dihydro-isoindole-2-carboxylic acid tert-butyl ester (0.033 g, 0.066 mmol, Example 42e) in methanol (3 mL) and dichloromethane (6 mL) for 15 minutes. The mixture was warmed to room temperature and stirred for 4 hours. The mixture was concentrated in vacuo, and the resulting solid was washed with hexanes and dried to afford the title compound (0.022 g, 85%) as a yellow solid, mp 180-185° C.: MSCI: m/z 400 (MH⁺).

### EXAMPLE 43

a) 2,4-Difluoro-3-methoxybenzamide

A 0° C. suspension of 2,4-difluoro-3-methoxybenzoic acid (147.8 g, 786 mmol, PCT Int. Appl. WO 9914214) in dichloromethane (1.5 L) was treated with oxalyl chloride (109.8 g, 865 mmol) followed by dimethylformamide (2 ml). The mixture was stirred at room temperature for 16 hours and the solvent removed in vacuo. The residual oil was dissolved in anhydrous tetrahydrofuran (500 ml) and added to a −70° C. solution of ether (1.5 L) saturated with ammonia gas. After the addition was complete, the reaction was allowed to come to room temperature where it was stirred for 1 hour. After diluting with ethyl acetate, the mixture was washed with water, brine, dried with magnesium sulfate, filtered and concentrated in vacuo to give 137.5 g of the title compound, mp 118-120 C. ¹H NMR (200 MHz, DMSO-d₆): δ 7.77 (bs, 1H), 7.70 (bs, 1H), 7.32-7.43 (m, 1H), 7.16-7.26 (m, 1H), 3.93 (s, 3H).

b) 2,4-Difluoro-3-hydroxybenzamide

A −70° C. solution of 2,4-difluoro-3-methoxybenzamide (124.0 g, 663 mmol, Example 43a) in methylene chloride (2.5 L) was treated dropwise, over 1 hour, with boron tribromide (338.0 g, 1350 mmol). The reaction was stirred at room temperature for 18 hours, cooled to −70° C., quenched with water and diluted with ethyl acetate, tetrahydrofuran and brine. The organic layer was separated and the aqueous layer was extracted with a mixture of ethyl acetate and tetrahydrofuran (2:1). The combined extracts were dried over anhydrous sodium sulfate, filtered and concentrated in vacuo affording 102.6 g of the title compound, mp 161-162° C. ¹H NMR (400 MHz, DMSO-d₆): δ 10.41 (s, 1H), 7.69 (bs, 1H), 7.62 (bs, 1H), 7.03-7.15 (m, 2H).

c) 3-Difluoromethoxydifluorobenzamide

In a steel bomb, a −70° C. mixture of 2,4-difluoro-3-hydroxybenzamide (173.0 g, 1000 mmol, Example 43b), potassium carbonate (165.6 g, 1200.0 mmol) and dimethylformamide (500 ml) was treated with a solution of dichlorodifluoromethane (1200 g, 1400 mmol) in N,N-dimethylformamide (800 ml). The reaction mixture was heated at 110° C. for 41 h, cooled to room temperature and the contents of the steel bomb added to a mixture of ethyl acetate (2 L) and water (4 L). The mixture was adjusted to pH 2 with 6N hydrochloric acid, the organic layer was separated and aqueous layer was extracted copiously with ethyl acetate. The combined organics were washed with brine, dried with sodium sulfate, filtered and concentrated under vacuum. Recrystallization of the residue (hexane/ethyl acetate) yielded 159.6 g of the title compound, mp 124° C.

¹H NMR (400 MHz, DMSO-d₆): δ 7.87 (bs, 1H), 7.79 (bs, 1H), 7.59-7.70 (m, 1H), 7.32-7.43(m, 1H), 7.28 (t, 1H).

d) 1-Cyclopropyl-3-(3-difluoromethoxydifluorobenzoyl)urea

A solution of 3-difluoromethoxydifluorobenzamide (60.0 g, 257 mmol, Example 43c), oxalyl chloride (98.0 g, 773 mmol) and 1,2-dichloroethane (330 ml) was heated at reflux for 4 hours. The solvent was removed in vacuo to give an oil that was used without further purification. A 0° C. solution of the oil in dioxane (330 mL) was treated rapidly dropwise with a solution of cyclopropylamine (33 ml, 470 mmol) in dioxane (330 ml). After stirring at room temperature for 1.5 hours, the mixture was diluted with water, extracted with ethyl acetate and the organic extracts washed with brine, dried with sodium sulfate, filtered and evaporated under in vacuo to give 66.0 g of the title compound, mp 128-130° C. ¹H NMR (400 MHz, DMSO-d₆): δ 10.90 (s, 1H), 8.27 (d, 1H), 7.58-7.69 (m, 1H), 7.37-7.47 (m, 1H), 7.27 (t, 1H), 2.63-2.75 (m, 1H), 0.62-0.75 (m, 2H), 0.53-0.59 (m, 2H).

e) 1-Cyclopropyl-8-difluoromethoxy-7-fluoro-1H-quinazolinedione

A solution of 1-cyclopropyl-3-(3-difluoromethoxydifluorobenzoyl)urea (60.0 g, 195 mmol, Example 43d) in a mixture of tetrahydrofuran (1 L) and N,N-dimethylformamide (75 ml) was treated portionwise with sodium hydride (30.0 g, 60% in mineral oil, 750 mol). After heating at reflux for 5 hours, the cooled reaction was poured over ice, adjusted to pH 2 with 6N hydrochloric acid and extracted with ethyl acetate. The organic extracts were washed with brine, dried with sodium sulfate, filtered and concentrated in vacuo. Purification of the residue by chromatography on silica gel (hexane/ethyl acetate 1:1) afforded 21.0 g of the title compound, mp 215-221° C. ¹H NMR (400 MHz, CDCl₃) δ 8.80 (s, 1H), 8.16 (m, 1H), 7.14 (m, 1H), 6.42 (t, 1H), 3.24 (m, 11H), 1.21 (m, 2H), 0.71 (m, 2H).

f) {(S)-1-[(R)-1-(1-Cyclopropyl-8-difluoromethoxydioxo-1,2,3,4-tetrahydroquinazolin-7-yl)pyrrolidin-3-yl]ethyl}carbamic Acid Tert-Butyl Ester

A mixture of 1-cyclopropyl-8-difluoromethoxy-7-fluoro-1H-quinazolinedione (0.2 g, 0. mmol, Example 43e), ((S)-(R)-1-pyrrolidin-3-ylethyl)carbamic acid tert-butyl ester (0.225 g, 1.97 mmol) and dimethyl sulfoxide (2 mL) was heated at 90° C. for 1.5 hours. The solution was treated with saturated ammonium chloride, stirred for 1 hour and filtered. The collected solid was washed with water and dried to afford the title compound (0.317 g). MSCI: m/z 481 (MH⁺).

g) 7-[(R)-3-((S)-1-Aminoethyl)pyrrolidin-1-yl]-1-cyclopropyl-8-difluoromethoxy-1H-quinazolinedione Hydrochloride

A solution of {(S)-1-[(R)-1-(1-cyclopropyl-8-difluoromethoxydioxo-1,2,3,4-tetrahydroquinazolin-7-yl)pyrrolidin-3-yl]ethyl} carbamic acid tert-butyl ester (0.317 g, Example 43f) in methanol (2 mL) was treated with a 2 M diethyl ether solution of hydrogen chloride (5 mL, 10 mmol) and allowed to stir for 1 hour. The mixture was concentrated in vacuo, re-dissolved in water and lyophilized to provide a solid (0.190 g); mp>250° C.; MSCI: m/z 381 (MH⁺).

### EXAMPLE 44

a) [(3R, 4S)- and (3S, 4R)-1-(1-Cyclopropyl-8-difluoromethoxy-6-fluorodioxo-1,2,3,4-tetrahydroquinazolin-7-yl)-4-trifluoromethylpyrrolidin-3-ylmethyl]carbamic Acid Tert-Butyl Ester

A mixture of 1-cyclopropyl-6,7-difluoro-8-difluoromethoxy-1H-quinazolinedione (0.20 g, 0.66 mmol, Example 39b), ((3S, 4S)- and (3R, 4R)-4-trifluoromethylpyrrolidin-3-ylmethyl)carbamic acid, tert-butyl ester (0.225 g, 1.97 mmol, [Bioorg. Med. Chem. Lett. 1998, 8, 2833]), triethylamine (0.28 mL, 2.0 mmol) and dimethyl sulfoxide (1 mL) was heated at 90° C. for 2 hours. The solution was treated with saturated ammonium chloride, stirred for 2 hours and filtered. The collected solid was washed with water and dried to afford the title compound (0.372 g). MSCI: m/z 553 (MH⁺).

b) 7-((3R, 4S)- and (3S, 4R)-3-Aminomethyl-4-trifluoromethylpyrrolidin-1-yl)-1-cyclopropyl-8-difluoromethoxy-6-fluoro-1H-quinazolinedione Hydrochloride

A solution of [(3R, 4S)- and (3S, 4R)-1-(1-Cyclopropyl-8-difluoromethoxy-6-fluorodioxo-1,2,3,4-tetrahydroquinazolin-7-yl)-4-trifluoromethylpyrrolidin-3-ylmethyl]carbamic acid tert-butyl ester (0.317 g, Example 44a) in methanol (5 mL) was treated with a 2 M diethyl ether solution of hydrogen chloride (6 mL, 12 mmol) and allowed to stir for 4 hours. The mixture was concentrated in vacuo, re-dissolved in water and lyophilized to provide a solid (0.298 g). mp 189-190° C.; MSCI: m/z 453 (MH⁺).

### GENERAL PROCEDURE FOR EXAMPLES 45-68

### Array Chemistry

In a 2-dram vial, a 0.33 M solution of the template (0.300 mL, 1 mmol, [1-cyclopropyl-6,7-difluoro-8-methyl-1H-quinazolinedione or 1-cyclopropyl-6,7-difluoro-8-methoxy-1H-quinazolinedione]) was treated with a 1 M solution of 300 μL of side chain (0.30 mL, 0.3 mmol) and 1,1,3,3-tetramethylguanidine (0.024 mL, 0.2 mmol). The mixture was shaken at 90° C. for 20 hours. Upon completion, the solution was concentrated using a Genevac concentrator (HT-12). The products were filtered through a silica gel plug eluting with 20% ethyl acetate in methylene chloride, to provide clean product.

Any product that used ethyl-[(S)-(R)-1-pyrrolidin-3-ylethyl]amine or methyl-[(S)-(R)-1-pyrrolidin-3-ylethyl]amine, as the side chain, was treated, prior to purification, with a 0.3 M solution of di-t-butyl dicarbonate (0.069 mL,0.3 mmol) in dichloromethane overnight at room temperature. The mixture was then concentrated using a Genevac concentrator (HT-12) and the product purified by filtration through a silica gel plug eluting with 20% ethyl acetate in dichloromethane.

The product was then treated with 2 ml of a saturated solution hydrogen chloride in methanol, at room temperature, overnight. The solution was then concentrated, as above, and purified by high pressure liquid chromatography (gradient: 10 to 100% 3% n-propanol in acetonitrile/3% aqueous n-propanol). The compound was analyzed by LC-MS.

### EXAMPLE 45

1-Cyclopropyl-6-fluoro-8-methoxy-7-[3(R)-(1(S)-methylaminoethyl)pyrrolidin-1-yl]-1H-quinazolinedione from 1-cyclopropyl-6,7-difluoro-8-methoxy-1H-quinazolinedione and [3(R)-(1(S)-methylaminoethyl)pyrrolidin-1-yl]carbamic acid tert-butyl ester; MSCI: m/z 377 (MH⁺).

### EXAMPLE 46

1-Cyclopropyl-6-fluoro-8-methyl-7-[3(R)-(1(S)-methylaminoethyl)pyrrolidin-1-yl]-1H-quinazolinedione, from 1-cyclopropyl-6,7-difluoro-8-methyl-1H-quinazolinedione and [3(R)-(1(S)-methylaminoethyl)pyrrolidin-1-yl]carbamic acid tert-butyl ester; MSCI: m/z 361 (MH⁺).

### EXAMPLE 47

7-(3-Aminopiperidin-1-yl)-1-cyclopropyl-6-fluoro-8-methoxy-1H-quinazolinedione, from 1-cyclopropyl-6,7-difluoro-8-methoxy-1H-quinazolinedione and piperidin-3-yl-carbamic acid tert-butyl ester [J. Med. Chem. 1995, 38(22), 4478.]; MSCI: m/z 349 (MH⁺).

### EXAMPLE 48

1-Cyclopropyl-6-fluoro-8-methoxy-7-(octahydropyrrolo[3,4-c]pyridin-2-yl)-1H-quinazolinedione, from 1-cyclopropyl-6,7-difluoro-8-methoxy-1H-quinazolinedione and octahydopyrrolo[3,4-c]pyridine-5-carboxylic acid tert-butyl ester [WO 0153273 A1]; MSCI: m/z 375 (MH⁺).

### EXAMPLE 49

7-((S)-3-Aminopyrrolidin-1-yl)-1-cyclopropyl-6-fluoro-8-methyl-1H-quinazolinedione, from 1-cyclopropyl-6,7-di fluoro-8-methyl-1H-quinazolinedione and (S)-pyrrolidin-3-ylcarbamic acid tert-butyl ester; MSCI: m/z 319 (MH⁺).

### EXAMPLE 50

7-(3-Aminopiperidin-1-yl)-1-cyclopropyl-6-fluoro-8-methyl-1H-quinazolinedione from 1-cyclopropyl-6,7-difluoro-8-methyl-1H-quinazolinedione and piperidin-3-ylcarbamic acid tert-butyl ester; MSCI: m/z 333 (MH⁺).

### EXAMPLE 51

1-Cyclopropyl-6-fluoro-8-methyl-7-(octahydropyrrolo[3,4-c]pyridin-2-yl)-1H-quinazolinedione, from 1-cyclopropyl-6,7-difluoro-8-methyl-1H-quinazolinedione and octahydropyrrolo[3,4-c]pyridine-1-carboxylic acid tert-butyl ester [WO 0153273 A1]; MSCI: m/z 359 (MH⁺).

### EXAMPLE 52

7-(3(S)-Aminopyrrolidin-1-yl)-1-cyclopropyl-6-fluoro-8-methoxy-1H-quinazolinedion, from 1-cyclopropyl-6,7-difluoro-8-methoxy-1H-quinazolinedione and 3(S)-pyrrolidin-3-ylcarbamic acid tert-butyl ester; MSCI: m/z 335 (MH⁺).

### EXAMPLE 53

7-(3-Aminomethyl-3-methylpyrrolidin-1-yl)-1-cyclopropyl-6-fluoro-8-methoxy-1H-quinazolinedione, from 1-cyclopropyl-6,7-difluoro-8-methoxy-1H-quinazolinedione and (3-methylpyrrolidin-3-ylmethyl)carbamic acid tert-butyl ester [J. Med. Chem. 1992, 35(2), 361]; MSCI: m/z 363 (MH⁺).

### EXAMPLE 54

7-(3-Aminomethylpyrrolidin-1-yl)-1-cyclopropyl-6-fluoro-8-methoxy-1H-quinazolinedione, from 1-cyclopropyl-6,7-difluoro-8-methoxy-1H-quinazolinedione and (pyrrolidin-3-ylmethyl)carbamic acid tert-butyl ester [EP 591030 A2]; MSCI: m/z 349 (MH⁺).

### EXAMPLE 55

7-[3(R)-(1-Amino-1-methylethyl)pyrrolidin-1-yl]-1-cyclopropyl-6-fluoro-8-methoxy-1H-quinazolinedione, from 1-cyclopropyl-6,7-difluoro-8-methoxy-1H-quinazolinedione and (3(R)-methylpyrrolidin-3-ylmethyl)carbamic acid tert-butyl ester; MSCI: m/z 377 (MH⁺).

### EXAMPLE 56

7-(3-Aminomethylpiperidin-1-yl)-1-cyclopropyl-6-fluoro-8-methoxy-1H-quinazolinedione, from 1-cyclopropyl-6,7-difluoro-8-methoxy-1H-quinazolinedione and piperidine-3-ylmethylcarbamic acid tert-butyl ester; MSCI: m/z 363 (MH⁺).

### EXAMPLE 57

7-(3-Aminomethyl-3-benzylpyrrolidin-1-yl)-1-cyclopropyl-6-fluoro-8-methoxy-1H-quinazolinedione, from 1-cyclopropyl-6,7-difluoro-8-methoxy-1H-quinazolinedione and (3-benzylpyrrolidin-3-ylmethyl)carbamic acid, tert-butyl ester [WO 0153273 A1]; MSCI: m/z 440 (MH⁺).

### EXAMPLE 58

1-Cyclopropyl-6-fluoro-8-methoxy-7-(octahydropyrrolo[3,4-b]pyridin-6-yl)-1H-quinazolinedione, from 1-cyclopropyl-6,7-difluoro-8-methoxy-1H-quinazolinedione and octahydropyrrolo[3,4-b]pyridine-1-carboxylic acid tert-butyl ester [JP 2001213878 A2]; MSCI: m/z 375 (MH⁺).

### EXAMPLE 59

7-(1-Amino-5-aza-spiro[2.4]hept-5-yl)-1-cyclopropyl-6-fluoro-8-methoxy-1H-quinazolinedione, from 1-cyclopropyl-6,7-difluoro-8-methoxy-1H-quinazolinedione and (5-azaspiro[2.4]hept-1-yl)carbamic acid tert-butyl ester [EP 550016 A1]; MSCI: m/z 361 (MH⁺)

### EXAMPLE 60

7-(3-Aminomethyl-3-methylpyrrolidin-1-yl)-1-cyclopropyl-6-fluoro-8-methyl-1H-quinazolinedione, from 1-cyclopropyl-6,7-difluoro-8-methyl-1H-quinazolinedione and (3-methylpyrrolidin-3-ylmethyl)carbamic acid tert-butyl ester; MSCI: m/z 347 (MH⁺).

### EXAMPLE 61

7-[3(R)-(1-Amino-1-methylethyl)pyrrolidin-1-yl]-1-cyclopropyl-6-fluoro-8-methyl-1H-quinazolinedione from 1-cyclopropyl-6,7-difluoro-8-methyl-1H-quinazolinedione and [(R)-1-methyl-1-pyrrolidin-3-ylethyl]carbamic acid tert-butyl ester [J. Med. Chem. 1994, 37(6), 733]; MSCI: m/z 3361 (MH⁺).

### EXAMPLE 62

1-Cyclopropyl-6-fluoro-8-methyl-7-(octahydropyrrolo[3,4-b]pyridin-6-yl)-1H-quinazolinedione from 1-cyclopropyl-6,7-difluoro-8-methyl-1H-quinazolinedione and octahydropyrrolo[3,4-b]pyridine-1-carboxylic acid tert-butyl ester; MSCI: m/z 359 (MH⁺).

### EXAMPLE 63

7-(3a-Aminomethyloctahydroisoindol-2-yl)-1-cyclopropyl-6-fluoro-8-methyl-1H-quinazolinedione, from 1-cyclopropyl-6,7-difluoro-8-methyl-1H-quinazolinedione and (octahydroisoindol-3a-ylmethyl)carbamic acid tert-butyl ester [WO 0153273 A1]; MSCI: m/z 387 (MH⁺).

### EXAMPLE 64

7-(3S, 4R)- and 7-((3R, 4S)-3-Amino-4-fluoromethylpyrrolidin-1-yl)-1-cyclopropyl-6-fluoro-8-methoxy-1H-quinazolinedione, from 1-cyclopropyl-6,7-difluoro-8-methoxy-1H-quinazolinedione and (3R,4S)- and (3S,4R)-4-fluoromethylpyrrolidin-3-yl)carbamic acid tert-butyl ester [Bioorg. Med. Chem. Lett. 1998, 8(15), 1953]; MSCI: m/z 367 (MH⁺)

### EXAMPLE 65

1-Cyclopropyl-7-[(R)-3-((S)-1-ethylaminoethyl)pyrrolidin-1-yl]-6-fluoro-8-methoxy-1H-quinazolinedione from 1-cyclopropyl-6,7-difluoro-8-methoxy-1 H-quinazolinedione and ethyl-[(S)-(R)-1-pyrrolidin-3-ylethyl]amine [J. Med. Chem. 1993, 36(7), 871]; MSCI: m/z 391 (MH⁺).

### EXAMPLE 66

7-(3a-Aminomethyloctahydroisoindol-2-yl)-1-cyclopropyl-6-fluoro-8-methoxy-1H-quinazolinedione, from 1-cyclopropyl-6,7-difluoro-8-methoxy-1H-quinazolinedione and (octahydroisoindol-3a-ylmethyl)carbamic acid tert-butyl ester; MSCI: m/z 403 (MH⁺).

### EXAMPLE 67

7-(3S, 4R)- and 7-((3R, 4S)-3-Amino-4-fluoromethylpyrrolidin-1-yl)-1-cyclopropyl-6-fluoro-8-methyl-1H-quinazolinedione, from 1-cyclopropyl-6,7-difluoro-8-methyl-1H-quinazolinedione and (3S, 4R)- and (3R, 4S)-4-fluoromethylpyrrolidin-3-yl)carbamic acid tert-butyl ester [Bioorg. Med. Chem. Lett. 1998, 8(15), 19531; MSCI: m/z 351 (MH⁺).

### EXAMPLE 68

1-Cyclopropyl-7-[(R)-3-((S)-1-ethylaminoethyl)pyrrolidin-1-yl]-6-fluoro-8-methyl-1H-quinazolinedione, from 1-cyclopropyl-6,7-difluoro-8-methyl-1H-quinazolinedione and ethyl-[(S)-(R)-1-pyrrolidin-3-ylethyl]amine; MSCI: m/z 375 (MH⁺).

### EXAMPLE 69

a) 1-Cyclopropyl-6,7-difluoro-8-methoxy-5-methyl-1H-quinazolinedione

A solution of 1-cyclopropyl-6,7-difluoro-8-methoxy-1H-quinazolinedione (1.0 g, 3.7 mmol) in tetrahydrofuran (18 mL) was cooled to −30° C. and treated with a 2 M heptane/tetrahydrofuran/ethylbenzene solution of lithium diisopropylamine (5.6 mL, 1 mmol). The mixture was allowed to stir for 2 hours then iodomethane (0.28 mL, 4.5 mmol) was added. After stirring for 1 hour and warming to 0° C., the mixture was quenched into water and extracted with ethyl acetate. The extracts were combined, dried with magnesium sulfate and purified via silica column chromatography (98:2 methylene chloride/methanol) to provide a solid (0.650 g). MSCI: m/z 283 (MH⁺).

b) {(S)-1-[(R)-1-(1-Cyclopropyl-6-fluoro-8-methoxy-5-methyldioxo-1,2,3,4-tetrahydro-quinazolin-7-yl)pyrrolidin-3-yl]ethyl}carbamic Acid Tert-Butyl Ester

1-Cyclopropyl-6,7-difluoro-8-methoxy-5-methyl-1H-quinazolinedione (0.19 g, 0.67 mmol, Example 69a), 1,1,3,3-tetramethylguanidine (0.17 mL, 1.4 mmol) and ((S)-(R)-1-pyrrolidin-3-ylethyl)carbamic acid tert-butyl ester (0.29 g, 1.4 mmol) in dimethyl sulfoxide (1.5 mL) were heated at 90° C. overnight. The solution was diluted with brine and extracted with ethyl acetate. The organic layers were then combined, dried over magnesium sulfate, filtered and concentrated. The residue was purified by flash silica gel chromatography (3:2 ethyl acetate/hexanes then 7:3 ethyl acetate/hexanes) to afford the title compound (0.280 g). MSCI: m/z 477 (MH⁺).

c) 3-((S)-1-Aminoethyl)pyrrolidin-1-yl]-1-cyclopropyl-6-fluoro-8-methoxy-5-methyl-1H-quinazolinedione Hydrochloride

To a solution of {(S)-1-[(R)-1-(1-Cyclopropyl-6-fluoro-8-methoxy-5-methyldioxo-1,2,3,4-tetrahydro-quinazolin-7-yl)pyrrolidin-3-yl]ethyl} carbamic acid tert-butyl ester (280 g, Example 69b) in dichloromethane (5 mL) was bubbled in hydrochloric and the resulting saturated solution was allowed to stir for 3 hours. The mixture was then concentrated, triturated with diethyl ether and dried to afford the title compound (0.21 g); mp 200-210° C.; MSCI: m/z 377 (MH⁺).

The following additional examples illustrate typical formulations for use according to the invention.

### EXAMPLE 70

The following illustrates representative pharmaceutical dosage forms, containing a compound of Formula I (“Invention Compound”), for therapeutic or prophylactic use in humans.

| (i) Tablet | mg/tablet |
|---|---|
| ‘Invention Compound’ | 25.0 |
| Lactose | 50.0 |
| Corn Starch (for mix) | 10.0 |
| Corn Starch (paste) | 10.0 |
| Magnesium Stearate (1%) | 3.0 |
| | 300.0 |

The biphenylsulfonamide, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) Tablet | mg/capsule |
|---|---|
| ‘Invention Compound | 10.0 |
| Colloidal Silicon Dioxide | 1.5 |
| Lactose | 465.5 |
| Pregelatinized Starch | 120.0 |
| Magnesium Stearate (1%) | 3.0 |
| | 600.0 |

| (iii) Preparation for | |
|---|---|
| Oral Solution | Amount |
| ‘Invention Compound’ | 400 mg |
| Sorbitol Solution (70% N.F.) | 40 mL |
| Sodium Benzoate | 20 mg |
| Saccharin | 5 mg |
| Cherry Flavor | 20 mg |
| Distilled Water q.s. | 100 mL |

The sorbitol solution is added to 40 mL of distilled water, and the biphenylsulfonamide is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| (v) Injection 1 (1 mg/mL) | Amount |
|---|---|
| ‘Invention Compound’ | 1.0 |
| Dibasic Sodium Phosphate | 12.0 |
| Monobasic Sodium Phosphate | 0.7 |
| Sodium Chloride | 4.5 |
| 1.0 N Sodium hydroxide solution | q.s. |
| (pH adjustment to 7.0-7.5) | |
| Water for injection | q.s. ad 1 mL |

| | Amount |
|---|---|
| (vi) Injection 2 (10 mg/mL) | |
| ‘Invention Compound’ | 10.0 |
| Dibasic Sodium Phosphate | 1.1 |
| Monobasic Sodium Phosphate | 0.3 |
| Polyethylene glyco 400 | 200.0 |
| 0.1 N hydrochloric acid solution | q.s. |
| (pH adjustment to 7.0-7.5) | |
| Water for injection | q.s. ad 1 mL |
| (vii) Injection 2 (10 mg/mL) | |
| ‘Invention Compound’ | 20.0 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of Formula I: 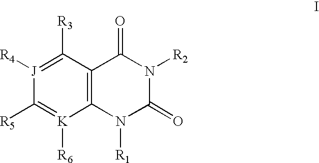
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 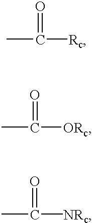
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, R₄, and R₆ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 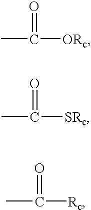
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 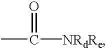
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;
R₁ and R₆ taken together with the atoms to which they are attached form a 5, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 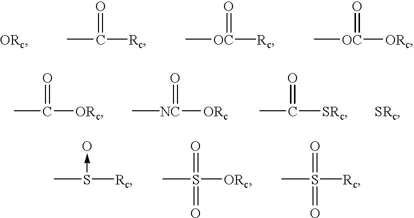
wherein R_{c} is defined as above, 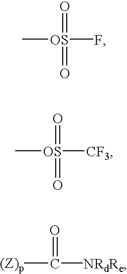
wherein Z is O or N R_{d} and Rₑ are defined as above and p is 0 or 1;
halo,
NO₂,
CN,
NR_{f}R_{g} wherein R_{f} and R_{g} are defined as for Rₐ and R_{b} above;
aryl or fused aryl,
heterocyclic or fused heterocyclic,
heteroaryl or fused heteroaryl,
bicyclic heterocyclic or Spiro heterocyclic,
wherein fused aryl, fused heterocyclic, fused heteroaryl, bicyclic heterocyclic, or spiro heterocyclic can be substituted; and
wherein J and K independently are C or N, provided that when J or K is N, R₄ or R₆ is absent at that position.

2. The compound of claim 1 wherein R₅ is selected from:

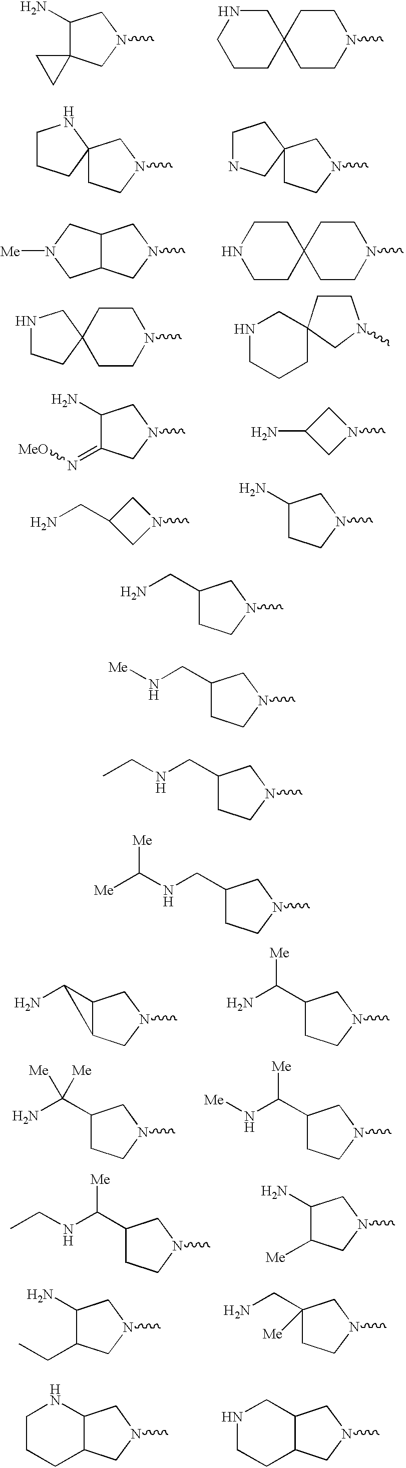

3. The compound of claim 1, wherein
J and K are C;
R₁ is methyl, ethyl, cyclopropyl, t-butyl, 2-fluorocyclopropyl;
R₂ is H;
R₃ is H. F Me, or NH₂;
R₄ is F or Cl;
R₅ is 1-pyrrolidinyl or substituted 1-pyrrolidinyl,
1-piperidinyl or substituted piperidinyl,
1-piperizinyl or substituted 1-piperizinyl or 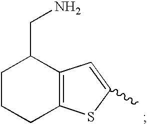
and
R₆ is F, Cl, methyl, methoxy, OCF₃, OCHF₂, OCH₂F, OCH₂CF₃, OCH₂CHF₂, or OCH₂CH₂F.

4. The compound of claim 1, wherein
J is N;
K is C;
R₁ is cyclopropyl;
R₂ is H;
R₃ is H, F, Me, or NH₂;
R₄ is F or Cl;
R₅ is 1-pyrrolidinyl or substituted 1-pyrrolidinyl,
1-piperidinyl or substituted 1-piperidinyl,
1-piperizinyl or substituted 1-piperizinyl, or 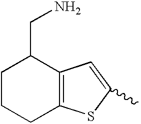
and
R₆ is F, Cl, methyl, methoxy, or OCF₃.

5. The compound of claim 1, wherein
J is C;
K is N;
R is cyclopropyl;
R₂ is H;
R₃ is H, F, Me, or NH₂;
R₄ is F or Cl; and
R₅ is 1-pyrrolidinyl or substituted 1-pyrrolidinyl,
1-piperidinyl or substituted 1-piperidinyl,
1-piperizinyl or substituted 1-piperizinyl, or 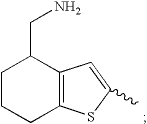
and
R₆ is F, Cl, methyl, methoxy, or OCF₃.

6. The compound of claim 1, wherein
J is N;
K is N;
R₁ is cyclopropyl;
R₂ is H;
R₃ is H, F, Me, or NH₂;
R₅ is 1-pyrrolidinyl or substituted 1-pyrrolidinyl,
1-piperidinyl or substituted 1-piperidinyl,
1-piperizinyl or substituted 1-piperizinyl, or 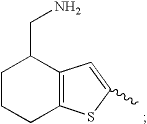
and
R₆ is F, Cl, methyl, methoxy, or OCF₃.

7. A compound of Formula II: 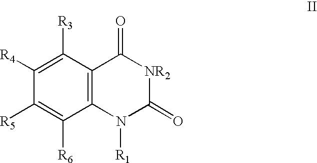
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 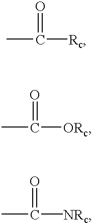
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, R₄, and R₆ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 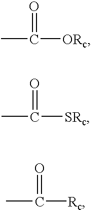
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 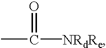
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;
R₁ and R₆ taken together with the atoms to which they are attached to form a 5, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 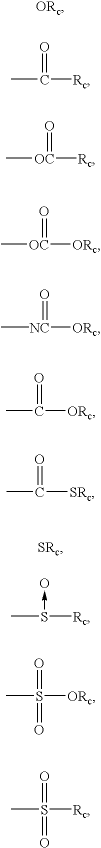
wherein R_{c} is defined as above, 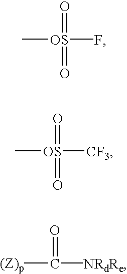
wherein Z is O or N R_{d} and Rₑ are defined as above and p is 0 or 1;
halo,
NO₂,
CN,
NR_{f}R_{g}, wherein R_{f} and R_{g} are defined as for Rₐ and R_{b} above;
aryl or fused aryl, f
heterocyclic or fused heterocyclic,
heteroaryl or fused heteroaryl, or
bicyclic heterocyclic or spiro heterocyclic;
wherein fused aryl, fused heterocyclic, fused heteroaryl, bicyclic heterocyclic, or spiro heterocyclic can be substituted.

8. The compound of claim 7, wherein R₅ is as provided in claim 2.

9. The compound of claim 7, wherein R₁-R₆ are as provided in claim 3.

10. The compound of claim 7 wherein
R₁ is cyclopropyl;
R₂ is H;
R₃ is H, F, Me, OMe, or NH₂;
R₄ is F or Cl;
R₅ is 1-pyrrolidinyl or substituted 1-pyrrolidinyl,
1-piperidinyl or substituted 1-piperidinyl,
1-piperizinyl or substituted 1-piperizinyl, or 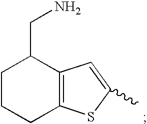
and
R₆ is F, Cl, methyl, methoxy, OCF₃, OCHF₂, OCH₂F, OCH₂CF₃ , OCH₂CHF₂, or OCH₂CH₂F.

11. The compound of claim 7, wherein
R₁ is cyclopropyl;
R₂ is H;
R₃ is H, F, Me, or NH₂;
R₄ is F;
R₅ is 1-pyrrolidinyl or substituted 1-pyrrolidinyl,
1-piperidinyl or substituted 1-piperidinyl,
1-piperizinyl or substituted 1-piperizinyl; and
R₆ is methyl, methoxy, or OCF₃.

12. The compound of claim 7, wherein
R₁ is cyclopropyl;
R₂ is H;
R₃ is H, F, Me, or NH₂;
R₄ is F;
R₅ is 1-pyrrolidinyl or substituted 1-pyrrolidinyl,
or 1-piperidinyl or substituted 1-piperidinyl; and
R₆ is F, Cl, methyl, methoxy, or OCF₃.

13. The compound of claim 7, wherein
R₁ is cyclopropyl;
R₂ is H;
R₃ is H, F, Me, or NH₂;
R₄ is F;
R₅ is 1-pyrrolidinyl; and R₆ is F, Cl, methyl, methoxy, or OCF₃.

14. A compound of Formula III: 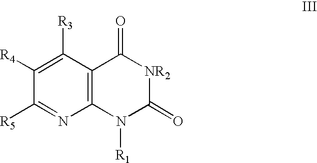
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 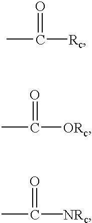
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, and R₄ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 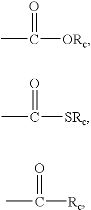
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 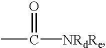
wherein R_{d} and Rₑ are independently H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 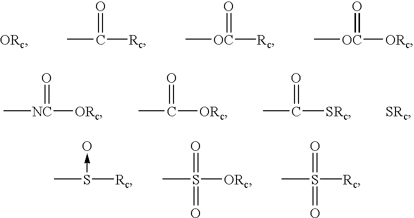
wherein R_{c} is defined as above, 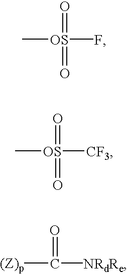
wherein Z is O or N R_{d} and Rₑ are defined as above and p is 0 or 1;
halo,
NO₂,
CN,
NR_{f}R_{g}, wherein R_{f} and R_{g} are defined as for Rₐ and R_{b} above;
aryl or fused aryl,
heterocyclic or fused heterocyclic,
heteroaryl or fused heteroaryl, or
bicyclic heterocyclic or spiro heterocyclic,
wherein fused aryl, fused heterocyclic, fused heteroaryl, bicyclic heterocyclic, or spiro heterocyclic can be substituted.

15. The compound of claim 14, wherein R₅ is as provided in claim 2.

16. The compound of claim 14, wherein
R₁ is cyclopropyl;
R₃ is H, F, Me, or NH₂;
R₄ is F or Cl; and
R₅ is 1-pyrrolidinyl or substituted 1-pyrrolidinyl,
1-piperidinyl or substituted 1-piperidinyl,
1-piperizinyl or substituted 1-piperizinyl, or

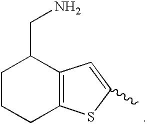

17. The compound of claim 14, wherein
R₁ is cyclopropyl;
R₂ is H;
R₃ is H, F, Me, OMe, or NH₂;
R₄ is F; and
R₅ is 1-pyrrolidinyl or substituted 1-pyrrolidinyl,
1-piperidinyl or substituted 1-piperidinyl,
1-piperizinyl or substituted 1-piperizinyl.

18. The compound of claim 14, wherein R₁ is cyclopropyl;
R₂ is H;
R₃ is H, F, Me, or NH₂;
R₄ is F; and
R₅ is 1-pyrrolidinyl or substituted 1-pyrrolidinyl, or
1-piperidinyl or substituted 1-piperidinyl.

19. The compound of claim 14, wherein
R₁ is cyclopropyl;
R₂ is H;
R₃ is H, F, Me, or NH₂;
R₄ is F; and
R₅ is 1-pyrrolidinyl or substituted 1-pyrrolidinyl.

20. A compound of Formula IV: 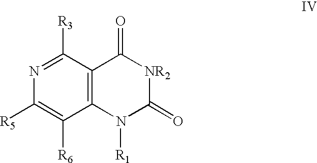
or a pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 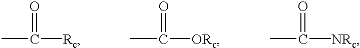
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃ and R₆ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 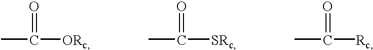
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 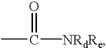
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;
R₁ and R₆ can be taken together with the atoms to which they are attached form a 5, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 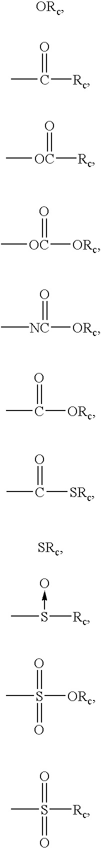
wherein R_{c} is defined as above, 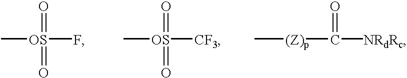
wherein Z is O or N R_{d} and Rₑ are defined as above and p is 0 or 1;
halo,
NO₂,
CN,
NR_{f}R_{g}, wherein R_{f} and R_{g} are defined as for Rₐ and R_{b} above;
aryl or fused aryl,
heterocyclic or fused heterocyclic,
heteroaryl or fused heteroaryl,
bicyclic heterocyclic or spiro heterocyclic,
wherein fused aryl, fused heterocyclic, fused heteroaryl, bicyclic heterocyclic, or spiro heterocyclic can be substituted.

21. The compound of claim 20, wherein R₅ is as provided in claim 2.

22. The compound of claim 20, wherein R₁ is cyclopropyl;
R₂ is H;
R₃ is H, methyl, NH₂, or methoxy;
R₅ is 1-pyrrolidinyl or substituted 1-pyrrolidinyl,
1-piperidinyl or substituted 1-piperidinyl,
1-piperizinyl or substituted 1-piperizinyl, or 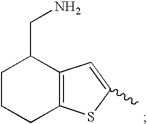
and
R₆ is F. Cl, methyl, methoxy, OCF₃, OCHF₂, OCH₂F, OCH₂CF₃, OCH₂CHF₂, or OCH₂CH₂F.

23. The compound of claim 20, wherein
R₁ is cyclopropyl;
R₂ is H;
R₃ is H, methyl, or methoxy;
R₅ is 1-pyrrolidinyl or substituted 1-pyrrolidinyl,
1-piperidinyl or substituted 1-piperidinyl,
1-piperizinyl or substituted 1-piperizinyl; and
R₆ is F, Cl, methyl, methoxy, or OCF₃.

24. The compound of claim 20, wherein
R₁ is cyclopropyl;
R₂ is H;
R₃ is H;
R₅ is 1-pyrrolidinyl or substituted 1-pyrrolidinyl, or
1-piperidinyl or substituted 1-piperidinyl; and
R₆ is F, Cl, methyl, methoxy, or OCF₃.

25. The compound of claim 20, wherein
R₁ is cyclopropyl;
R₂ is H;
R₃ is H;
R₅ is 1-pyrrolidinyl or substituted 1-pyrrolidinyl, or
1-piperidinyl or substituted 1-piperidinyl; and
R₆ is F, Cl, methyl, methoxy, or OCF₃.

26. A compound of Formula V: 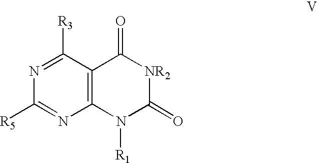
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 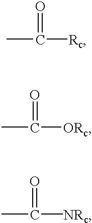
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃ is H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 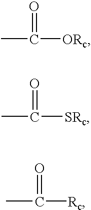
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 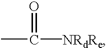
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 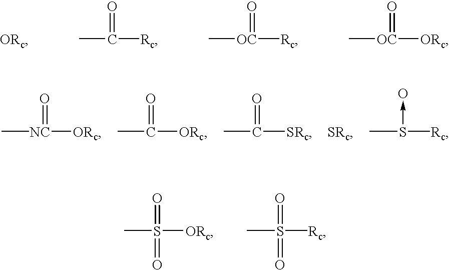
wherein R_{c} is defined as above, 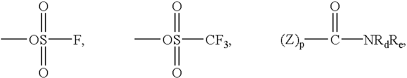
wherein Z is O or N R_{d} and Rₑ are defined as above and p is 0 or 1;
halo,
NO₂,
CN,
NR_{f}R_{g}, wherein R_{f} and R_{g} are defined as for Rₐ and R_{b} above;
aryl or fused aryl,
heterocyclic or fused heterocyclic,
heteroaryl or fused heteroaryl,
bicyclic heterocyclic or spiro heterocyclic,
wherein fused aryl, fused heterocyclic, fused heteroaryl, bicyclic heterocyclic, or spiro heterocyclic can be substituted.

27. The compound of claim 26, wherein R₅ is as provided in claim 2.

28. The compound of claim 26, wherein
R₁ is cyclopropyl;
R₂ is H;
R₃ is H, methyl, NH₂, or methoxy; and
R₅ is 1-pyrrolidinyl or substituted 1-pyrrolidinyl,
1-piperidinyl or substituted 1-piperidinyl,
1-piperizinyl or substituted 1-piperizinyl, or

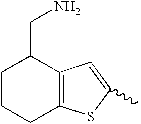

29. The compound of claim 26, wherein
R₁ is cyclopropyl;
R₂ is H;
R₃ is H, methyl, NH₂, or methoxy; and
R₅ is 1-pyrrolidinyl or substituted 1-pyrrolidinyl, or
1-piperidinyl or substituted 1-piperidinyl.

30. The compound of claim 26, wherein
R₁ is cyclopropyl;
R₂ is H;
R₃ is H, methyl, NH₂, or methoxy; and
R₅ is 1-pyrrolidinyl or substituted 1-pyrrolidinyl.

31. The compound of claim 26, wherein
R₁ is cyclopropyl;
R₃ is H, methyl, NH₂, or methoxy; and
R₅ is substituted 1-pyrrolidinyl.

32. A compound of formula VI: 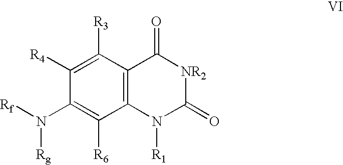
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 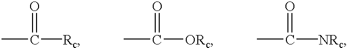
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, R₄, and R₆ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 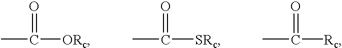
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 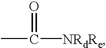
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 5, 6, 7, or 8 membered ring having from 0 to 3 heteroatoms selected from N, O, and S, wherein said ring is optionally substituted by one or more substituents;
R₁ and R₆ taken together with the atoms to which they are attached form a 5, 6, 7, or 8 membered ring having from 0 to 3 heteroatoms selected from N, O, and S, wherein said ring is optionally substituted by one or more substituents; and
R_{f} and R_{g} are defined as for Rₐand R_{b} above.

33. The compound of claim 32, wherein R_{f} and R_{g} together with the nitrogen to which they are attached form, any of the groups provided in claim 2.

34. The compound of claim 32, wherein
R₁ is cyclopropyl;
R₂ is H;
R₃ is H, methyl, NH₂, or methoxy;
R₄ is F or Cl;
R_{f} and R_{g}, together with the nitrogen to which they are attached, are
1-pyrrolidinyl or substituted 1-pyrrolidinyl,
1-piperidinyl or substituted 1-piperidinyl, or
1-piperizinyl or substituted 1-piperizinyl; and
R₆ is F, Cl, methyl, methoxy, OCF₃, OCHF₂, OCH₂F, OCH₂CF₃, OCH₂CHF₂, or OCH₂CH₂F.

35. The compound of claim 32, wherein
R₁ is cyclopropyl;
R₂ is H; R₃ is H, methyl, NH₂, or methoxy;
R₄ is F;
R_{f} and R_{g}, together with the nitrogen to which they are attached, are
1-pyrrolidinyl or substituted 1-pyrrolidinyl,
1-piperidinyl or substituted 1-piperidinyl,
1-piperizinyl or substituted 1-piperizinyl; and
R₆ is methyl, methoxy, OCF₃, OCHF₂, OCH₂F, or OCH₂CF₃.

36. The compound of claim 32, wherein
R₁ is cyclopropyl;
R₂ is H; R₃ is H, methyl, NH₂, or methoxy;
R₄ is F;
R_{f} and R_{g}, together with the nitrogen to which they are attached, are
1-pyrrolidinyl or substituted 1-pyrrolidinyl, or
1-piperidinyl or substituted 1-piperidinyl; and
R₆ is methyl, methoxy, or OCF₃.

37. The compound of claim 32, wherein
R₁ is cyclopropyl;
R₂ is H; R₃ is H, methyl, NH₂, or methoxy;
R₄ is F;
R_{f} and R_{g}, together with the nitrogen to which they are attached, are
1-pyrrolidinyl or substituted 1-pyrrolidinyl; and
R₆ is F, Cl, methyl, methoxy, or OCF₃.

38. A compound of formula VII: 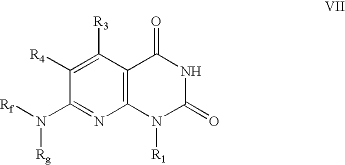
or a tautomer or pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 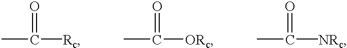
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, and R₄ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 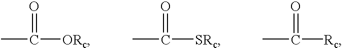
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 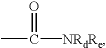
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 6, 7, or 8 membered ring having from 0 to 3 heteroatoms selected from N, O, and S, wherein said ring is optionally substituted by one or more substituents; and
R_{f} and R_{g} are defined as for Rₐand R_{b} above.

39. The compound of claim 38, wherein R_{f} and R_{g} together with the nitrogen to which they are attached form any of the groups provided in claim 2.

40. The compound of claim 38, wherein
R₁ is cyclopropyl;
R₃ is H, methyl, NH₂, or methoxy;
R₄ is F or Cl; and
R_{f} and R_{g}, together with the nitrogen to which they are attached, are
1-pyrrolidinyl or substituted 1-pyrrolidinyl,
1-piperidinyl or substituted 1-piperidinyl,
1-piperizinyl or substituted 1-piperizinyl.

41. The compound of claim 38, wherein
R₁ is cyclopropyl;
R₃ is H, methyl, NH₂, or methoxy;
R₄ is F; and
R_{f} and R_{g}, together with the nitrogen to which they are attached, are
1-pyrrolidinyl or substituted 1-pyrrolidinyl, or
1-piperidinyl or substituted 1-piperidinyl.

42. The compound of claim 38, wherein
R₁ is cyclopropyl;
R₃ is H, methyl, NH₂, or methoxy;
R₄ is F; and
R_{g} and Rₕ, together with the nitrogen to which they are attached, are
1-pyrrolidinyl or substituted 1-pyrrolidinyl.

43. The compound of claim 38, wherein
R₁ is cyclopropyl;
R₃ is H, methyl, NH₂, or methoxy;
R₄ is F; R_{f} and R_{g}, together with the nitrogen to which they are attached, are substituted 1-pyrrolidinyl.

44. A compound of formula VIII: 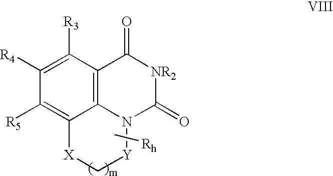
or a tautomer or pharmaceutically acceptable salt thereof wherein:
R₂ is H, 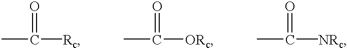
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃ and R₄ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 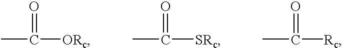
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 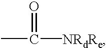
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 6, 7, or 8 membered ring having from 0 to 3 heteroatoms selected from N, O, and S, wherein said ring is optionally substituted by one or more substituents;
R₅ is hydrogen,
C₁-C₇ alkyl and substituted alkyl,
C₂-C₇ alkenyl and substituted alkenyl,
C₂-C₇ alkynyl and substituted alkynyl, 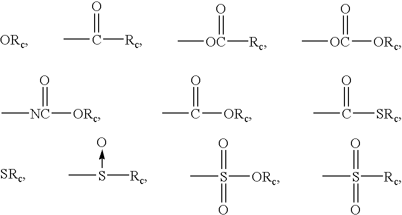
wherein R_{c} is defined as above, 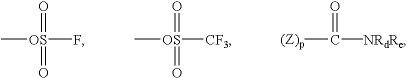
wherein Z is O or N R_{d} and Rₑ are defined as above and p is 0 or 1;
halo,
NO₂,
CN,
NR_{f}R_{g}, wherein R_{f} and R_{g} are defined as for Rₐ and R_{b} above;
aryl or fused aryl,
heterocyclic or fused heterocyclic,
heteroaryl or fused heteroaryl,
bicyclic heterocyclic or spiro heterocyclic,
wherein fused aryl, fused heterocyclic, fused heteroaryl, bicyclic heterocyclic, or spiro heterocyclic can be substituted;
X and Y each independently are O, CH₂, CH(C₁-C₇ alkyl), C(C₁-C₇ alkyl)₂, C(C₃-C₆ cycloalkyl), 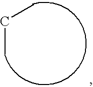
wherein 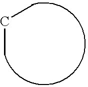
is a C₃-C₆ cycloalkyl, NH, N(C₁-C₇ alkyl), S, SO, or SO₂;
m is 0-14;
Rₕ is H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRⱼRₖ,
wherein Rⱼ and Rₖ independently are H,
C₁-C₇ alkyl and substituted alkyl,
C₂-C₇ alkenyl and substituted alkenyl,
C₂-C₇ alkynyl and substituted alkynyl, 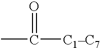
alkyl and substituted alkyl, or
Rⱼ and Rₖ taken together with the nitrogen to which they are attached form a 3- to 7-membered ring containing from 1 to 3 heteroatoms selected from N, O, and S, said ring being unsubstituted or substituted with 1, 2, 3, or 4 substituent groups.

45. The compound of claim 45, wherein R₅ is any of the groups provided in claim 2.

46. The compound of claim 44, wherein
m is 1;
X is O or CH₂;
Y is CH₂, CH(C₁-C₇ alkyl), NH, or N(C₁-C₇ alkyl);
Rₕ is methyl;
R₂ is H;
R₃ is H;
R₄ is F or Cl; and
R₅ is 1-pyrrolidinyl or substituted 1-pyrrolidinyl,
1-piperidinyl or substituted 1-piperidinyl, or
1-piperizinyl or substituted 1-piperizinyl.

47. The compound of claim 44, wherein
m is 1;
X is O or CH₂;
Y is CH₂, CH(C₁-C₇ alkyl), NH, or N(C₁-C₇ alky 1);
Rₕ is methyl;
R₂ is H;
R₃ is H; and
R₅ is 1-pyrrolidinyl or substituted 1-pyrrolidinyl, or
1-piperidinyl or substituted 1-piperidinyl.

48. The compound of claim 44, wherein
m is 1;
X is O or CH₂;
Y is CH₂, CH(C₁-C₇ alkyl), NH, or N(C₁-C₇ alkyl);
Rₕ is methyl;
R₂ is H;
R₃ is H;
R₄ is F or Cl; and
R₅ is 1-pyrrolidinyl or substituted 1-pyrrolidinyl.

49. The compound of claim 44, wherein
m is 1;
X is O or CH₂;
Y is CH₂, CH(C₁-C₇ alkyl), NH, or N(C₁-C₇ alkyl);
Rₕ is methyl;
R₂ is H;
R₃ is H; and
R₅ is substituted 1-pyrrolidinyl.

50. A compound of formula IX: 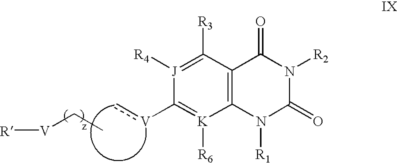
or a pharmaceutically acceptable salt thereof wherein:
R₁ is H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
aryl and substituted aryl,
heterocyclic and substituted heterocyclic,
or heteroaryl and substituted heteroaryl;
R₂ is H, 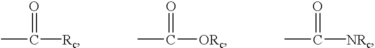
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above;
R₃, R₄, and R₆ independently are H,
OH,
(O)ₙC₁-C₇ alkyl and substituted alkyl,
(O)ₙC₂-C₇ alkenyl and substituted alkenyl,
(O)ₙC₂-C₇ alkynyl and substituted alkynyl,
wherein n is 0 or 1,
halo,
NO₂,
CN,
NRₐR_{b}, wherein Rₐ and R_{b} are each independently H,
C₁-C₇ alkyl and substituted alkyl,
C₂-C₇ alkenyl and substituted alkenyl,
C₂-C₇ alkynyl and substituted alkynyl,
C₃-C₇ cycloalkyl and substituted cycloalkyl,
C₅-C₈ cycloalkenyl and substituted cycloalkenyl,
aryl and substituted aryl, or 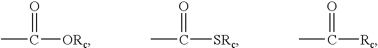
wherein R_{c} is
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl,defined as above; 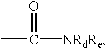
wherein R_{d} and Rₑ are independantly H,
C₁-C₇ alkyl and substituted alkyl,
C₂-C₇ alkenyl and substituted alkenyl,
C₃-C₇ cycloalkyl and substituted cycloalkyl,
aryl and substituted aryl,
heteroaryl and substituted heteroaryl,
heterocycloalkyl and substituted heterocycloalkyl;
aryl and substituted aryl,
heteroaryl and substituted heteroaryl,
heterocycloalkyl and substituted heterocycloalkyl, or
Rₐ and R_{b} taken together with the nitrogen to which they are attached form a 4, 5, 6, 7, or 8 membered ring having from 0 to 3 heteroatoms selected from N, O, and S, wherein said ring is optionally substituted by one or more substituents;
R₁ and R₆ taken together with the atoms to which they are attached form a 5, 6, 7, or 8 membered ring having from 0 to 3 heteroatoms selected from N, O, and S, wherein said ring is optionally substituted by one or more substituents; 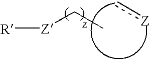
is aryl or fused aryl,
heterocyclic or fused heterocyclic,
heteroaryl or fused heteroaryl,
bicyclic heterocyclic or spiro heterocyclic,
wherein fused aryl, fused heterocyclic, fused heteroaryl, bicyclic heterocyclic, or spiro heterocyclic can be substituted;
V is N, CH, or C, provided that when Z is N or CH, “- -” is absent and when Z is C, “ [symbol] ” is a double bond;
z is 0, 1, 2, or 3;
V′ is O, S, NH₂, NHR″, wherein R″ is C₁-C₇ alkyl and substituted alkyl;
R′ is 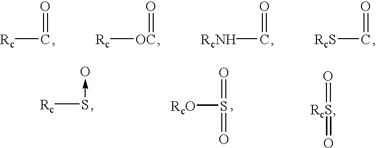
wherein R_{c} is defined as above, 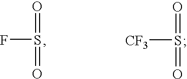
and
wherein J and K independently are C or N, provided that when J or K is N, R₄ or R₆ is absent at that position.

51. The invention also provides a compound which is:
7-(6-amino-3-aza-bicyclo[3.1.0]hex-3-yl)-6-fluoro-3H-1-methylcyclopropyl-1H-quinazoline-2,4-dione (1α, 5α, 6α) hydrochloride,
1-Cyclopropyl-6-fluoro-8-methyl-7-[(R)-3-((S)-1-methylaminoethyl)-pyrrolidin-1-yl]-1H-quinazoline-2,4-dione,
1-Cyclopropyl-6-fluoro-8-methoxy-7-[(R)-3-((S)-1-methylaminoethyl)-pyrrolidin-1-yl]-1H-quinazoline-2,4-dione,
7-[(R)-3-((S)-1-Aminoethyl)pyrrolidin-1-yl]-1-cyclopropyl-6-fluoro-8-methyl-1H-quinazoline-2,4-dione hydrochloride,
1-Cyclopropyl-7-dimethylamino-6-fluoro-8-methyl-1H-quinazoline-2,4-dione,
7-((S)-3-Amino-pyrrolidin-1-yl)-8-chloro-1-cyclopropyl-6-fluoro-1H-quinazoline-2,4-dione trifluoroacetic acid,
7-(3-[1-Amino-1-(2-fluorophenyl)methyl]-pyrrolidin-1-yl}-1-cyclopropyl-6-fluoro-6-methyl-1H-quinazoline-2,4-dione hydrochloride,
1-Cyclopropyl-8-methyl-7-[(R)-3-((S)-1-methylaminoethyl)pyrrolidin-1-yl]-1H-pyrido[4,3-d]pyrimidine-2,4-dione hydrochloride,
7-((S)-3-Aminopyrrolidin-1-yl)-1-cyclopropyl-6-fluoro-1H-pyrido[2,3-d]pyrimidine-2,4-dione hydrochloride,
7-[(R)-3-((S)-1-Aminoethyl)pyrrolidin-1-yl]-1-cyclopropyl-6-fluoro-1H-pyrido[2,3-d]pyrimidine-2,4-dione hydrochloride,
7-((S)-3-Aminopyrrolidin-1-yl)-8-fluoro-5-methyl-5,6-dihydropyrrolo[3,2,1-ij]quinazoline-1,3-dione hydrochloride,
7-[(R)-3-((S)-1-Aminoethyl)pyrrolidin-1-yl)-8-fluoro-5-methyl-5,6-dihydropyrrolo[3,2, 1-i,j] quinazoline-1,3-dione hydrochloride, 8-((S)-3-Aminopyrrolidin-1-yl)-9-fluoro-5-methyl-6,7-dihydropyrido[3,2, 1-ij]quinazoline-1,3-dione hydrochloride,
8-[(R)-3-((S)-1-Aminoethyl)pyrrolidin-1-yl)-9-fluoro-5-methyl-6,7-dihydro-5H-pyrido[3,2,1-i,j] quinazoline-1,3-dione hydrochloride,
1-(1-Cyclopropyl-6-fluoro-8-methyl-2,4-dioxo-1,2,3,4-tetrahydroquinazolin-7-yl)cyclopropanecarbonitrile,
1-(1-Cyclopropyl-6-fluoro-8-methyl-2,4-dioxo-1,2,3,4-tetrahydroquinazolin-7-yl)cyclopropanecarboxylic acid amide,
7-Amino-9-[9-(R)-3-((S)-1-aminoethyl)pyrrolidin-1-yl)-8-fluoro-3-methyl-2,3-dihydro-1-oxa-3a,5-diazaphenalene-4,6-dione hydrochloride,
7-((3aR, 6aS)- and (3aS, 6aR)-4-Aminohexahydrocyclopenta[c]pyrrol-2-yl-1-cyclopropyl-6-fluoro-8-methyl-1H-quinazoline-2,4-dione, hydrochloride,
7-((3aR, 6aS)- and (3aS, 6aR)-4-Aminohexahydrocyclopenta[c]pyrrol-2-yl-1-cyclopropyl-6-fluoro-8-methoxy-1H-quinazoline-2,4-dione hydrochloride,
7-[3-(1-Amino-2-fluoroethyl)pyrrolidin-1-yl]-1-cyclopropyl-6-fluoro-8-methyl-1H-quinazoline-2,4-dione hydrochloride,
7-[3-(Aminocyclopropylmethyl)-pyrrolidin-1-yl]-1-cyclopropyl-6-fluoro-8-methyl-1H-quinazoline-2,4-dione hydrochloride,
7-(3-Aminomethyl-4-fluoromethylpyrrolidin-1-yl)-1-cyclopropyl-6-fluoro-8-methyl-1H-quinazoline-2,4-dione,
7-(5-Aminomethylthiophen-3-yl)-1-cyclopropyl-6-fluoro-8-methyl-1H-quinazoline-2,4-dione hydrochloride,
1-Cyclopropyl-7-(5,5-difluoro-4-hydroxy-4,5,6,7-tetrahydrobenzo[b]thiophen-2-yl)-6-fluoro-8-methyl-1H-quinazoline-2,4-dione,
7-(4-Amino-5,5-difluoro-4,5,6,7-tetrahydrobenzo[b]thiophen-2-yl)-1-cyclopropyl-6-fluoro-8-methyl-1H-quinazoline-2,4-dione hydrochloride,
7-(4-Amino-5,6-dihydro-4H-cyclopenta[b]thiophen-2-yl)-1-cyclopropyl-6-fluoro-8-methyl-1H-quinazoline-2,4-dione hydrochloride,
7-[(R)-3-(1-Aminocyclopropyl)pyrrolidin-1-yl]-1-cyclopropyl-6-fluoro-8-methyl-1H-quinazoline-2,4-dione hydrochloride,
7-(4-Amino-5,6-dihydro-4H-4,5,6,7-tetrahydrobenzo[b]-thiophen-7-yl)-1-cyclopropyl-6-fluoro-8-methyl-1H-quinazoline-2,4-dione hydrochloride,
1-cyclopropyl-6-fluoro-8-methyl-7-(7-methyl-4,5,6,7-tetrahydrothieno[2,3-c]pyridin-2-yl)-1H-quinazoline-2,4-dione,
1-Cyclopropyl-6-fluoro-8-methyl-7-(4-methyl-5,6-dihydro-4H-thieno[2,3-c]pyrrol-2-yl)-1H-quinazoline-2,4-dione hydrochloride,
7-[[(3S, 4R)-3-(R)- and (3R, 4S)-3-(S)]-1-amino-2,2,2-trifluoroethyl)-4-hydroxypyrrolidin-1-yl]-1-cyclopropyl-6-fluoro-8-methyl-1H-quinazoline-2,4-dione,
7-[3-(Aminooxazol-4-ylmethyl)pyrrolidin-1-yl]-1-cyclopropyl-6-fluoro-8-methyl-1H-quinazoline-2,4-dione,
7-(3R, 4S)- and 7-((3S, 4R)-3-Aminomethyl-4-fluoropyrrolidin-1-yl)-1-cyclopropyl-6-fluoro-8-methoxy-1H-quinazoloine-2,4-dione hydrochloride,
7-(3-Aminohexahydrofuro[2,3-c]pyrrol-5-yl)-1-cyclopropyl-6-fluoro-8-methyl-1H-quinazoline-2,4-dione hydrochloride,
7-[4-(Aminoethyl)-3,3-dimethylpyrrolidin-1-yl]-1-cylcopropyl-6-fluoro-8-methyl-1H-quinazoline-2,4-dione hydrochloride,
7-(4-Aminooctahydroisoindol-2-yl)-1-cyclopropyl-6-fluoro-8-methyl-1H-quinazoline-2,4-dione hydrochloride,
7-[(R)-3-((S)-1-Aminoethyl)pyrrolidin-1-yl]-1-cyclopropyl-6-fluoro-8-fluoromethoxy-1H-quinazoline-2,4-dione hydrochloride,
7-[(R)-3-((S)-1-Aminoethyl)pyrrolidin-1-yl]-1-cyclopropyl-8-difluoromethyl-6-fluoro-1H-quinazoline-2,4-dione hydrochloride,
7-[5-(1-Aminocyclopropyl)thiophen-2-yl]-1-cyclopropyl-6-fluoro-8-methyl-1H-quinazoline-2,4-dione hydrochloride,
7-[(R)-3-(1-Aminocyclopropyl)pyrrolidin-1-yl]-1-cyclopropyl-8-diflouromethoxy-6-fluoro-1H-quinazoline-2,4-dione,
7-[(R)-3-((S)-1-Aminoethyl)pyrrolidin-1-yl]-1-cyclopropyl-6-fluoro-8-difluoromethoxy-1H-quinazoline-2,4-dione hydrochloride,
7-[(R)-3-((S)-1-Aminoethyl)pyrrolidin-1-yl]-1-cyclopropyl-6-fluoro-5,8-dimethyl-1H-quinazoline-2,4-dione hydrochloride,
1-Cyclopropyl-8-difluoromethoxy-7-((R)-1-methyl-2,3-dihydro-1H-isoindol-5-yl)-1H-quinazoline-2,4-dione hydrochloride,
7-[(R)-3-((S)-1-Aminoethyl)pyrrolidin-1-yl]-1-cyclopropyl-8-difluoromethoxy-1H-quinazoline-2,4-dione hydrochloride,
7-((3R, 4S)- and (3S, 4R)-3-Aminomethyl-4-trifluoromethylpyrrolidin-1-yl)-1-cyclopropyl-8-difluoromethoxy-6-fluoro-1H-quinazoline-2,4-dione hydrochloride,
1-Cyclopropyl-6-fluoro-8-methoxy-7-[3(R)-1(S)-methylaminoethyl)pyrrolidin-1-yl]-1H-quinazoline-2,4-dione,
1-Cyclopropyl-6-fluoro-8-methyl-7-[3(R)-(1(S)-methylaminoethyl)pyrrolidin-1-yl]-1H-quinazoline-2,4-dione,
7-(3-Aminopiperidin-1-yl)-1-cyclopropyl-6-fluoro-8-methoxy-1H-quinazoline-2,4-dione,
1-Cyclopropyl-6-fluoro-8-methoxy-7-(octahydropyrrolo[3,4-c]pyridin-2-yl)-1H-quinazoline-2,4-dione,
7-((S)-3-Aminopyrrolidin-1-yl)-1-cyclopropyl-6-fluoro-8-methyl-1H-quinazoline-2,4-dione,
7-(3-Aminopiperidin-1-yl)-1-cyclopropyl-6-fluoro-8-methyl-1H-quinazoline-2,4-dione,
1-Cyclopropyl-6-fluoro-8-methyl-7-(octahydropyrrolo[3,4-c]pyridin-2-yl)-1H-quinazoline-2,4-dione,
7-(3(S)-Aminopyrrolidin-1-yl)-1-cyclopropyl-6-fluoro-8-methoxy-1H-quinazoline-2,4-dione,
7-(3-Aminomethyl-3-methylpyrrolidin-1-yl)-1-cyclopropyl-6-fluoro-8-methoxy-1H-quinazoline-2,4-dione,
7-(3-Aminomethylpyrrolidin-1-yl)-1-cyclopropyl-6-fluoro-8-methoxy-1H-quinazoline-2,4-dione,
7-[3(R)-(1-Amino-1-methylethyl)-pyrrolidin-1-yl]-1-cyclopropyl-6-fluoro-8-methoxy-1H-quinazoline-2,4-dione,
7-(3-Aminomethylpiperidin-1-yl)-1-cyclopropyl-6-fluoro-8-methoxy-1H-quinazoline-2,4-dione,
7-(3-Aminomethyl-3-benzylpyrrolidin-1-yl)-1-cyclopropyl-6-fluoro-8-methoxy-1H-quinazoline-2,4-dione,
1-Cyclopropyl-6-fluoro-8-methoxy-7-(octahydropyrrolo[3,4-b]pyridin-6-yl)-1H-quinazoline-2,4-dione,
7-(1-Amino-5-aza-spiro[2.4]hept-5-yl)-1-cyclopropyl-6-fluoro-8-methoxy-1H-quinazoline-2,4-dione,
7-(3-Aminomethyl-3-methylpyrrolidin-1-yl)-1-cyclopropyl-6-fluoro-8-methyl-1H-quinazoline-2,4-dione,
7-[3(R)-(1-Amino-1-methylethyl)pyrrolidin-1-yl]-1-cyclopropyl-6-fluoro-8-methyl-1H-quinazoline-2,4-dione,
1-Cyclopropyl-6-fluoro-8-methyl-7-(octahydropyrrolo[3,4-b]pyridin-6-yl)-1H-quinazoline-2,4-dione,
7-(3a-Aminomethyloctahydroisoindol-2-yl)-1-cyclopropyl-6-fluoro-8-methyl-1H-quinazoline-2,4-dione,
7-(3S, 4R)- and 7-((3R, 4S)-3-Amino-4-fluoromethylpyrrolidin-1-yl)-1-cyclopropyl-6-fluoro-8-methoxy-1H-quinazoline-2,4-dione,
1-Cyclopropyl-7-[3(R)-(1-ethylaminoethyl)pyrrolidin-1-yl]-6-fluoro-8-methoxy-1H-quinazoline-2,4-dione,
7-(3a-Aminomethyloctahydroisoindol-2-yl)-1-cyclopropyl-6-fluoro-8-methoxy-1H-quinazoline-2,4-dione,
7-(3S, 4R)- and 7-((3R, 4S)-3-Amino-4-fluoromethylpyrrolidin-1-yl)-1-cyclopropyl-6-fluoro-8-methyl-1H-quinazoline-2,4-dione,
1-Cyclopropyl-7-[(R)-3-((S)-1-ethylaminoethyl)pyrrolidin-1-yl]-6-fluoro-8-methyl-1H-quinazoline-2,4-dione,
7-[(R)-3-((S)-1-Aminoethyl)pyrrolidin-1-yl]-1-cyclopropyl-6-fluoro-8-methoxy-5-methyl-1H-quinazoline-2,4-dione hydrochloride; or
a pharmaceutically acceptable salt thereof.

52. The compound which is:
7-[3-Aminocyclopropyl)-4-trifluoromethylpyrrolidin-1-yl]-1-cyclopropyl-6-fluoro-8-methoxy-5-methyl-1H-quinazoline-2,4-dione;
1-Cyclopropyl-6-fluoro-7-(3-hydroxymethylpyrrolidin-1-yl)-8-methoxy-5-methyl-1H-quinazoline-2,4-dione;
1-Cyclopropyl-6-fluoro-8-methoxy-5-methyl-7-pyrrolidin-1-yl-1H-quinazoline-2,4-dione;
7-[3-Aminopyrrolidin-1-yl]-1-cyclopropyl-6-fluoro-8-methoxy-5-methyl-1H-quinazoline-2,4-dione;
7-[3-(2-Amino-1-hydroxyethyl)pyrrolidin-1-yl]-1-cyclopropyl-6-fluoro-8-methoxy-5-methyl-1H-quinazoline-2,4-dione;
7-[3-(2-Amino-1-hydroxyethyl)-4-fluoropyrrolidin-1-yl]-1-cyclopropyl-6-fluoro-8-methoxy-5-methyl-1H-quinazoline-2,4-dione;
1-Cyclopropyl-6-fluoro-8-methoxy-5-methyl-7-morpholin-4-yl-1H-quinazoline-2,4-dione;
1-Cyclopropyl-6-fluoro-8-methoxy-5-methyl-7-piperazin-1-yl-1H-quinazoline-2,4-dione;
1-Cyclopropyl-7-{3-[(2,4-difluorophenyl)hydroxymethyl]-4-fluoropyrrolidin-1-yl}-6-fluoro-8-methoxy-5-methyl-1H-quinazoline-2,4-dione;
1-Cyclopropyl-7-{3-[(4-fluorophenyl)hydroxymethyl]-4-fluoropyrrol idin-1-yl}-6-fluoro-8-methoxy-5-methyl-1H-quinazoline-2,4-dione;
1-Cyclopropyl-6-fluoro-7-(4-hydroxyoctahydroisoindol-2-yl)-8-methoxy-5-methyl-1H-quinazoline-2,4-dione;
1-Cyclopropyl-6-fluoro-7-(4-hydroxyhexahydrocyclopent[c]pyrrol-2-yl)-8-methoxy-5-methyl-1H-quinazoline-2,4-dione;
5-Amino-7-[3-aminocyclopropyl)-4-trifluoromethylpyrrolidin-1-yl]-1-cyclopropyl-6-fluoro-8-methoxy-1H-quinazoline-2,4-dione;
5-Amino-1-cyclopropyl-6-fluoro-7-(3-hydroxymethylpyrrolidin-1-yl)-8-methoxy-1H-quinazoline-2,4-dione;
5-Amino-1-cyclopropyl-6-fluoro-8-methoxy-7-pyrrolidin-1-yl-1H-quinazoline-2,4-dione;
5-Amino-7-[3-aminopyrrolidin-1-yl]-1-cyclopropyl-6-fluoro-8-methoxy-1H-quinazoline-2,4-dione;
5-Amino-7-[3-(2-amino-1-hydroxyethyl)pyrrolidin-1-yl]-1-cyclopropyl-6-fluoro-8-methoxy-1H-quinazoline-2,4-dione;
5-Amino-7-[3-(2-amino-1-hydroxyethyl)-4-fluoropyrrolidin-1-yl]-1-cyclopropyl-6-fluoro-8-methoxy-1H-quinazoline-2,4-dione;
5-Amino-1-cyclopropyl-6-fluoro-8-methoxy-7-morpholin-4-yl-1H-quinazoline-2,4-dione;
5-Amino-1-cyclopropyl-6-fluoro-8-methoxy-7-piperazin-1-yl-1H-quinazoline-2,4-dione;
5-Amino-1-cyclopropyl-7-{3-[(2,4-difluorophenyl)hydroxymethyl]-4-fluoropyrrolidin-1-yl}-6-fluoro-8-methoxy-1H-quinazoline-2,4-dione;
5-Amino-1-cyclopropyl-7-{3-[(4-fluorophenyl)hydroxymethyl]-4-fluoropyrrolidin-1-yl}-6-fluoro-8-methoxy-1H-quinazoline-2,4-dione;
5-Amino-1-cyclopropyl-6-fluoro-7-(4-hydroxyoctahydroisoindol-2-yl)-8-methoxy-1H-quinazoline-2,4-dione;
5-Amino-1-cyclopropyl-6-fluoro-7-(4-hydroxyhexahydrocyclopent[c]pyrrol-2-yl)-8-methoxy-1H-quinazoline-2,4-dione;
7-[3-Aminocyclopropyl)-4-trifluoromethylpyrrolidin-1-yl]-1-cyclopropyl-6-fluoro-5-hydroxy-8-methoxy-1H-quinazoline-2,4-dione;
1-Cyclopropyl-6-fluoro-7-(3-hydroxymethylpyrrolidin-1-yl)-5-hydroxy-8-methoxy-1H-quinazoline-2,4-dione;
1-Cyclopropyl-6-fluoro-5-hydroxy-8-methoxy-7-pyrrolidin-1-yl-1H-quinazoline-2,4-dione;
7-[3-Aminopyrrolidin-1-yl]-1-cyclopropyl-6-fluoro-5-hydroxy-8-methoxy-1H-quinazoline-2,4-dione;
7-[3-(2-Amino-1-hydroxyethyl)pyrrolidin-1-yl]-1-cyclopropyl-6-fluoro-5-hydroxy-8-methoxy-1H-quinazoline-2,4-dione;
7-[3-(2-Amino-1-hydroxyethyl)-4-fluoropyrrolidin-1-yl]-1-cyclopropyl-6-fluoro-5-hydroxy-8-methoxy-1H-quinazoline-2,4-dione;
1-Cyclopropyl-6-fluoro-5-hydroxy-8-methoxy-7-morpholin-4-yl-1H-quinazoline-2,4-dione;
1-Cyclopropyl-6-fluoro-5-hydroxy-8-methoxy-7-piperazin-1-yl-1H-quinazoline-2,4-dione;
1-Cyclopropyl-7-{3-[(2,4-difluorophenyl)hydroxymethyl]-4-fluoropyrrolidin-1-yl}-6-fluoro-5-hydroxy-8-methoxy-1H-quinazoline-2,4-dione;
1-Cyclopropyl-7-{3-[(4-fluorophenyl)hydroxymethyl]-4-fluoropyrrolidin-1-yl}-6-fluoro-5-hydroxy-8-methoxy-1H-quinazoline-2,4-dione;
1-Cyclopropyl-6-fluoro-7-(4-hydroxyoctahydroisoindol-2-yl)-5-hydroxy-8-methoxy-1H-quinazoline-2,4-dione;
1-Cyclopropyl-6-fluoro-7-(4-hydroxyhexahydrocyclopent[c]pyrrol-2-yl)-5-hydroxy-8-methoxy-1H-quinazoline-2,4-dione;
7-[3-(1-Aminocyclopropyl)-4-trifluoromethylpyrrolidin-1-yl]-1-cyclopropyl-5-difluoromethyl-6-fluoro-8-methoxy-1H-quinazoline-2,4-dione;
1-Cyclopropyl-5-difluoromethyl-6-fluoro-7-(3-hydroxymethylpyrrolidin-1-yl)-8-methoxy-1H-quinazoline-2,4-dione;
7-(3-Aminopyrrolidin-1-yl)-1-cyclopropyl-5-difluoromethyl-6-fluoro-8-methoxy-1H-quinazoline-2,4-dione;
7-[3-(1-Amino-2-hydroxyethyl)-4-fluoropyrrolidin-1-yl]-1-cyclopropyl-5-difluoromethyl-6-fluoro-8-methoxy-1H-quinazoline-2,4-dione;
1-Cyclopropyl-5-difluoromethyl-6-fluoro-8-methoxy-7-pyrrolidin-1-yl-1H-quinazoline-2,4-dione;
7-[3-(2-Amino-1-hydroxyethyl)pyrrolidin-1-yl]-1-cyclopropyl-5-difluoromethyl-6-fluoro-8-methoxy-1H-quinazoline-2,4-dione;
1-Cyclopropyl-5-difluoromethyl-6-fluoro-8-methoxy-7-piperazin-1-yl-1H-quinazoline-2,4-dione;
1-Cyclopropyl-5-difluoromethyl-6-fluoro-8-methoxy-7-morpholin-4-yl-1H-quinazoline-2,4-dione;
1-Cyclopropyl-5-di fluoromethyl-6-fluoro-7-{3-fluoro-4-[(4-fluorophenyl)-hydroxymethyl]pyrrolidin-1-yl}-8-methoxy-1H-quinazoline-2,4-dione;
1-Cyclopropyl-5-difluoromethyl-7-{3-[(2,4-difluorophenyl)hydroxymethyl]-4-fluoropyrrolidin-1-yl}-6-fluoro-8-methoxy-1H-quinazoline-2,4-dione;
1-Cyclopropyl-5-difluoromethyl-6-fluoro-7-(4-hydroxyoctahydroisoindol-2-yl)-8-methoxy-1H-quinazoline-2,4-dione;
1-Cyclopropyl-5-difluoromethyl-6-fluoro-7-(4-hydroxyhexahydro-cyclopenta[c]pyrrol-2-yl)-8-methoxy-1H-quinazoline-2,4-dione;
7-(4-Aminooctahydrocyclohepta[c]pyrrol-2-yl)-1-cyclopropyl-5-difluoromethyl-6-fluoro-8-methoxy-1H-quinazoline-2,4-dione;
1-Cyclopropyl-6-fluoro-7-(3-hydroxymethylpyrrolidin-1-yl)-5,8-dimethyl-1H-quinazoline-2,4-dione;
7-(3-Aminopyrrolidin-1-yl)-1-cyclopropyl-6-fluoro-5,8-dimethyl-1H-quinazoline-2,4-dione;
7-[3-(1-Amino-2-hydroxyethyl)-4-fluoropyrrolidin-1-yl]-1-cyclopropyl-6-fluoro-5,8-dimethyl-1H-quinazoline-2,4-dione;
1-Cyclopropyl-6-fluoro-5,8-dimethyl-7-pyrrolidin-1-yl-1H-quinazoline-2,4-dione;
7-[3-(2-Amino-1-hydroxyethyl)pyrrolidin-1-yl]-1-cyclopropyl-6-fluoro-5,8-dimethyl-1H-quinazoline-2,4-dione;
1-Cyclopropyl-6-fluoro-5,8-dimethyl-7-piperazin-1-yl-1H-quinazoline-2,4-dione;
1-Cyclopropyl-6-fluoro-5,8-dimethyl-7-morpholin-4-yl-1H-quinazoline-2,4-dione;
1-Cyclopropyl-6-fluoro-7-{3-fluoro-4-[(4-fluorophenyl)hydroxymethyl]-pyrrolidin-1-yl}-5,8-dimethyl-1H-quinazoline-2,4-dione;
1-Cyclopropyl-7-{3-[(2,4-difluorophenyl)hydroxymethyl]-4-fluoropyrrolidin-1-yl}-6-fluoro-5,8-dimethyl-1H-quinazoline-2,4-dione;
1-Cyclopropyl-6-fluoro-7-(4-hydroxyoctahydroisoindol-2-yl)-5,8-dimethyl-1H-quinazoline-2,4-dione;
1-Cyclopropyl-6-fluoro-7-(4-hydroxyhexahydrocyclopenta[c]pyrrol-2-yl)-5,8-dimethyl-1H-quinazoline-2,4-dione;
1-Cyclopropyl-6-fluoro-7-(4-hydroxyoctahydrocyclohepta[c]pyrrol-2-yl)-5,8-dimethyl-1H-quinazoline-2,4-dione;
7-[3-(1-Aminocyclopropyl)-4-trifluoromethylpyrrolidin-1-yl]-1-cyclopropyl-6-fluoro-5-methoxy-8-methyl-1H-quinazoline-2,4-dione;
1-Cyclopropyl-6-fluoro-7-(3-hydroxymethylpyrrolidin-1-yl)-5-methoxy-8-methyl-1H-quinazoline-2,4-dione;
7-(3-Aminopyrrolidin-1-yl)-1-cyclopropyl-6-fluoro-5-methoxy-8-methyl-1H-quinazoline-2,4-dione;
7-[3-(1-Amino-2-hydroxyethyl)-4-fluoropyrrolidin-1-yl]-1-cyclopropyl-6-fluoro-5-methoxy-8-methyl-1H-quinazoline-2,4-dione;
1-Cyclopropyl-6-fluoro-5-methoxy-8-methyl-7-pyrrolidin-1-yl-1H-quinazoline-2,4-dione;
7-[3-(2-Amino-1-hydroxyethyl)pyrrolidin-1-yl]-1-cyclopropyl-6-fluoro-5-methoxy-8-methyl-1H-quinazoline-2,4-dione;
1-Cyclopropyl-6-fluoro-5-methoxy-8-methyl-7-piperazin-1-yl-1H-quinazoline-2,4-dione;
1-Cyclopropyl-6-fluoro-5-methoxy-8-methyl-7-morpholin-4-yl-1H-quinazoline-2,4-dione;
1-Cyclopropyl-6-fluoro-7-{3-fluoro-4-[(4-fluorophenyl)hydroxymethyl]-pyrrolidin-1-yl}-5-methoxy-8-methyl-1H-quinazoline-2,4-dione;
1-Cyclopropyl-7-{3-[(2,4-difluorophenyl)hydroxymethyl]-4-fluoropyrrolidin-1-yl}-6-fluoro-5-methoxy-8-methyl-1H-quinazoline-2,4-dione;
1-Cyclopropyl-6-fluoro-7-(4-hydroxy-octahydro-isoindol-2-yl)-5-methoxy-8-methyl-1H-quinazoline-2,4-dione;
1-Cyclopropyl-6-fluoro-7-(4-hydroxyhexahydrocyclopenta[c]pyrrol-2-yl)-5-methoxy-8-methyl-1H-quinazoline-2,4-dione;
1-Cyclopropyl-6-fluoro-7-(4-hydroxyoctahydrocyclohepta[c]pyrrol-2-yl)-5-methoxy-8-methyl-1H-quinazoline-2,4-dione;
7-(3-Aminomethylpyrrolidin-1-yl)-1-cyclopropyl-6-fluoro-8-methoxy-5-methyl-1H-quinazoline-2,4-dione;
7-[3-(1-Aminocyclopropyl)pyrrolidin-1-yl]-1-cyclopropyl-6-fluoro-8-methoxy-5-methyl-1H-quinazoline-2,4-dione;
7-[3-(1-Amino-1-methylethyl)pyrrolidin-1-yl]-1-cyclopropyl-6-fluoro-8-methoxy-5-methyl-1H-quinazoline-2,4-dione;
7-[3-(1-Amino-2-fluoroethyl)pyrrolidin-1-yl]-1-cyclopropyl-6-fluoro-8-methoxy-5-methyl-1H-quinazoline-2,4-dione;
7-[3-(1-Amino-2,2-difluoroethyl)pyrrolidin-1-yl]-1-cyclopropyl-6-fluoro-8-methoxy-5-methyl-1H-quinazoline-2,4-dione;
7-[3-(1-Amino-2,2,2-trifluoroethyl)pyrrolidin-1-yl]-1-cyclopropyl-6-fluoro-8-methoxy-5-methyl-1H-quinazoline-2,4-dione;
7-[3-(1-Aminoethyl)-4-fluoropyrrolidin-1-yl]-1-cyclopropyl-6-fluoro-8-methoxy-5-methyl-1H-quinazoline-2,4-dione;
7-{3-[Amino-(2,6-difluorophenyl)methyl]pyrrolidin-1-yl}-1-cyclopropyl-6-fluoro-8-methoxy-5-methyl-1H-quinazoline-2,4-dione;
7-(3-Aminomethyl-4-fluoromethylpyrrolidin-1-yl)-1-cyclopropyl-6-fluoro-8-methoxy-5-methyl-1H-quinazoline-2,4-dione;
7-[3-(Aminothiazol-2-yl-methyl)pyrrolidin-1-yl]-1-cyclopropyl-6-fluoro-8-methoxy-5-methyl-1H-quinazoline-2,4-dione;
7-[3-(Aminocyclopropylmethyl)pyrrolidin-1-yl]-1-cyclopropyl-6-fluoro-8-methoxy-5-methyl-1H-quinazoline-2,4-dione;
7-(4-Aminooctahydroisoindol-2-yl)-1-cyclopropyl-6-fluoro-8-methoxy-5-methyl-1H-quinazoline-2,4-dione;
7-(4-Aminooctahydrocyclohepta[c]pyrrol-2-yl)-1-cyclopropyl-6-fluoro-8-methoxy-5-methyl-1H-quinazoline-2,4-dione;
1-Cyclopropyl-6-fluoro-7-[3-(1-hydroxycyclopropyl)pyrrolidin-1-yl]-8-methoxy-5-methyl-1H-quinazoline-2,4-dione;
7-(4-Aminohexahydrocyclopenta[c]pyrrol-2-yl)-1-cyclopropyl-6-fluoro-8-methoxy-5-methyl-1H-quinazoline-2,4-dione;
7-[3-(Aminooxazol-4-yl-methyl)pyrrolidin-1-yl]-1-cyclopropyl-6-fluoro-8-methoxy-5-methyl-1H-quinazoline-2,4-dione;
5-Amino-7-(3-aminomethylpyrrolidin-1-yl)-1-cyclopropyl-6-fluoro-8-methoxy-1H-quinazoline-2,4-dione;
5-Amino-7-[3-(1-aminocyclopropyl)pyrrolidin-1-yl]-1-cyclopropyl-6-fluoro-8-methoxy-1H-quinazoline-2,4-dione;
5-Amino-7-[3-(1-amino-1-methylethyl)pyrrolidin-1-yl]-1-cyclopropyl-6-fluoro-8-methoxy-1H-quinazoline-2,4-dione;
5-Amino-7-[3-(1-amino-2-fluoroethyl)pyrrolidin-1-yl]-1-cyclopropyl-6-fluoro-8-methoxy-1H-quinazoline-2,4-dione;
5-Amino-7-[3-(1-amino-2,2-difluoroethyl)pyrrolidin-1-yl]-1-cyclopropyl-6-fluoro-8-methoxy-1H-quinazoline-2,4-dione;
5-Amino-7-[3-(1-amino-2,2,2-trifluoroethyl)pyrrolidin-1-yl]-1-cyclopropyl-6-fluoro-8-methoxy-1H-quinazoline-2,4-dione;
5-Amino-7-[3-(1-aminoethyl)-4-fluoropyrrolidin-1-yl]-1-cyclopropyl-6-fluoro-8-methoxy-1H-quinazoline-2,4-dione;
5-Amino-7-{3-[amino-(2,6-difluorophenyl)methyl]pyrrolidin-1-yl}-1-cyclopropyl-6-fluoro-8-methoxy-1H-quinazoline-2,4-dione;
5-Amino-7-(3-aminomethyl-4-fluoromethylpyrrolidin-1-yl)-1-cyclopropyl-6-fluoro-8-methoxy-1H-quinazoline-2,4-dione;
5-Amino-7-[3-(aminothiazol-2-yl-methyl)pyrrolidin-1-yl]-1-cyclopropyl-6-fluoro-8-methoxy-1H-quinazoline-2,4-dione;
5-Amino-7-[3-(aminocyclopropylmethyl)pyrrolidin-1-yl]-1-cyclopropyl-6-fluoro-8-methoxy-1H-quinazoline-2,4-dione;
5-Amino-7-(4-aminooctahydroisoindol-2-yl)-1-cyclopropyl-6-fluoro-8-methoxy-1H-quinazoline-2,4-dione;
5-Amino-7-(4-aminooctahydrocyclohepta[c]pyrrol-2-yl)-1-cyclopropyl-6-fluoro-8-methoxy-1H-quinazoline-2,4-dione;
5-Amino-1-cyclopropyl-6-fluoro-7-[3-(1-hydroxycyclopropyl)pyrrolidin-1-yl]-8-methoxy-1H-quinazoline-2,4-dione;
5-Amino-7-(4-aminohexahydrocyclopenta[c]pyrrol-2-yl)-1-cyclopropyl-6-fluoro-8-methoxy-1H-quinazoline-2,4-dione;
5-Amino-7-[3-(aminooxazol-4-yl-methyl)pyrrolidin-1-yl]-1-cyclopropyl-6-fluoro-8-methoxy-1H-quinazoline-2,4-dione;
7-(3-Aminomethylpyrrol idin-1-yl)-1-cyclopropyl-5-difluoromethyl-6-fluoro-8-methyl-1H-quinazoline-2,4-dione;
7-[3-(1-Aminocyclopropyl)pyrrolidin-1-yl]-1-cyclopropyl-5-difluoromethyl-6-fluoro-8-methyl-1H-quinazoline-2,4-dione;
7-[3-(1-Amino-1-methylethyl)pyrrolidin-1-yl]-1-cyclopropyl-5-difluoromethyl-6-fluoro-8-methyl-1H-quinazoline-2,4-dione;
7-[3-(1-Amino-2-fluoroethyl)pyrrolidin-1-yl]-1-cyclopropyl-5-difluoromethyl-6-fluoro-8-methyl-1H-quinazoline-2,4-dione;
7-[3-(1-Amino-2,2-difluoroethyl)pyrrolidin-1-yl]-1-cyclopropyl-5-difluoromethyl-6-fluoro-8-methyl-1H-quinazoline-2,4-dione;
7-[3-(1-Amino-2,2,2-trifluoroethyl)pyrrolidin-1-yl]-1-cyclopropyl-5-difluoromethyl-6-fluoro-8-methyl-1H-quinazoline-2,4-dione;
7-[3-(1-Amino-ethyl)-4-fluoro-pyrrolidin-1-yl]-1-cyclopropyl-5-difluoromethyl-6-fluoro-8-methyl-1H-quinazoline-2,4-dione;
1-Cyclopropyl-5-difluoromethyl-7-{3-[(2,6-difluorophenyl)hydroxymethyl]-pyrrolidin-1-yl}-6-fluoro-8-methyl-1H-quinazoline-2,4-dione;
7-(3-Aminomethyl-4-fluoromethylpyrrolidin-1-yl)-1-cyclopropyl-5-difluoromethyl-6-fluoro-8-methyl-1H-quinazoline-2,4-dione;
7-[3-(Aminothiazol-2-yl-methyl)pyrrolidin-1-yl]-1-cyclopropyl-5-difluoromethyl-6-fluoro-8-methyl-1H-quinazoline-2,4-dione;
7-[3-(Amino-cyclopropyl-methyl)-pyrrolidin-1-yl]-1-cyclopropyl-5-difluoromethyl-6-fluoro-8-methyl-1H-quinazoline-2,4-dione;
1-Cyclopropyl-5-difluoromethyl-6-fluoro-7-[3-(1-hydroxycyclopropyl)-pyrrolidin-1-yl]-8-methyl-1H-quinazoline-2,4-dione;
7-[3-(Aminooxazol-4-ylmethyl)pyrrolidin-1-yl]-1-cyclopropyl-5--difluoromethyl-6-fluoro-8-methyl-1H-quinazoline-2,4-dione;
7-(3-Aminomethylpyrrolidin-1-yl)-1-cyclopropyl-6-fluoro-5,8-dimethyl-1H-quinazoline-2,4-dione;
7-[3-(1-Aminocyclopropyl)pyrrolidin-1-yl]-1-cyclopropyl-6-fluoro-5,8-dimethyl-1H-quinazoline-2,4-dione;
7-[3-(1-Amino-2-fluoroethyl)pyrrolidin-1-yl]-1-cyclopropyl-6-fluoro-5,8-dimethyl-1H-quinazoline-2,4-dione;
7-[3-(1-Amino-2,2-difluoroethyl)pyrrolidin-1-yl]-1-cyclopropyl-6-fluoro-5,8-dimethyl-1H-quinazoline-2,4-dione;
7-[3-(1-Amino-2,2,2-trifluoroethyl)pyrrolidin-1-yl]-1-cyclopropyl-6-fluoro-5,8-dimethyl-1H-quinazoline-2,4-dione;
1-Cyclopropyl-7-{3-[(2,6-difluorophenyl)hydroxymethyl]pyrrolidin-1-yl}-6-fluoro-5,8-dimethyl-1H-quinazoline-2,4-dione;
7-(3-Aminomethyl-4-fluoromethylpyrrolidin-1-yl)-1-cyclopropyl-6-fluoro-5,8-dimethyl-1H-quinazoline-2,4-dione;
7-[3-(Aminothiazol-2-ylmethyl)pyrrolidin-1-yl]-1-cyclopropyl-6-fluoro-5,8-dimethyl-1H-quinazoline-2,4-dione;
7-[3-(Aminocyclopropylmethyl)pyrrolidin-1-yl]-1-cyclopropyl-6-fluoro-5,8-dimethyl-1H-quinazoline-2,4-dione;
1-Cyclopropyl-6-fluoro-7-[3-(1-hydroxycyclopropyl)pyrrolidin-1-yl]-5,8-dimethyl-1H-quinazoline-2,4-dione;
7-[3-(Aminooxazol-4-ylmethyl)pyrrol idin-1-yl]-1-cyclopropyl-6-fluoro-5,8-dimethyl-1H-quinazoline-2,4-dione;
5-Amino-7-(3-aminomethylpyrrolidin-1-yl)-1-cyclopropyl-6-fluoro-8-methyl-1H-quinazoline-2,4-dione;
5-Amino-7-[3-(1-aminocyclopropyl)pyrrolidin-1-yl]-1-cyclopropyl-6-fluoro-8-methyl-1H-quinazoline-2,4-dione;
5-Amino-7-[3-(1-amino-1-methylethyl)pyrrolidin-1-yl]-1-cyclopropyl-6-fluoro-8-methyl-1H-quinazoline-2,4-dione;
5-Amino-7-[3-(1-amino-2,2,2-trifluoroethyl)pyrrolidin-1-yl]-1-cyclopropyl-6-fluoro-8-methyl-1H-quinazoline-2,4-dione;
5-Amino-7-[3-(1-amino-2-fluoroethyl)pyrrolidin-1-yl]-1-cyclopropyl-6-fluoro-8-methyl-1H-quinazoline-2,4-dione;
5-Amino-7-[3-(1-aminoethyl)-4-fluoropyrrolidin-1-yl]-1-cyclopropyl-6-fluoro-8-methyl-1H-quinazoline-2,4-dione;
5-Amino-7-[3-(1-amino-2,2-difluoroethyl)pyrrolidin-1-yl]-1-cyclopropyl-6-fluoro-8-methyl-1H-quinazoline-2,4-dione;
5-Amino-1-cyclopropyl-7-{3-[(2,6-difluorophenyl)hydroxymethyl]pyrrolidin-1-yl}-6-fluoro-8-methyl-1H-quinazoline-2,4-dione;
5-Amino-7-(3-aminomethyl-4-fluoromethylpyrrolidin-1-yl)-1-cyclopropyl-6-fluoro-8-methyl-1H-quinazoline-2,4-dione;
5-Amino-7-[3-(aminothiazol-2-ylmethyl)pyrrolidin-1-yl]-1-cyclopropyl-6-fluoro-8-methyl-1H-quinazoline-2,4-dione;
5-Amino-1-cyclopropyl-6-fluoro-7-[3-(1-hydroxycyclopropyl)pyrrolidin-1-yl]-8-methyl-1H-quinazoline-2,4-dione;
5-Amino-7-[3-(aminooxazol-4-ylmethyl)pyrrolidin-1-yl]-1-cyclopropyl-6-fluoro-8-methyl-1H-quinazoline-2,4-dione;
7-[3-(1-Amino-2-fluoroethyl)pyrrolidin-1-yl]-1-cyclopropyl-6-fluoro-5-hydroxy-8-methyl-1H-quinazoline-2,4-dione;
7-[3-(1-Amino-2,2-difluoroethyl)pyrrolidin-1-yl]-1-cyclopropyl-6-fluoro-5-hydroxy-8-methyl-1H-quinazoline-2,4-dione;
7-[3-(1-Amino-2,2,2-trifluoroethyl)pyrrolidin-1-yl]-1-cyclopropyl-6-fluoro-5-hydroxy-8-methyl-1H-quinazoline-2,4-dione;
7-[3-(Aminothiazol-2-ylmethyl)pyrrolidin-1-yl]-1-cyclopropyl-6-fluoro-5-hydroxy-8-methyl-1H-quinazoline-2,4-dione;
1-Cyclopropyl-6-fluoro-5-hydroxy-7-[3-(1-hydroxycyclopropyl)pyrrolidin-1-yl]-8-methyl-1H-quinazoline-2,4-dione;
7-[3-(Aminooxazol-4-ylmethyl)pyrrolidin-1-yl]-1-cyclopropyl-6-fluoro-5-hydroxy-8-methyl-1H-quinazoline-2,4-dione;
7-[3-(1-Aminocyclopropyl)pyrrolidin-1-yl]-1-cyclopropyl-6-fluoro-5-methoxy-8-methyl-1H-quinazoline-2,4-dione;
7-[3-(1-Amino-2-fluoroethyl)pyrrolidin-1-yl]-1-cyclopropyl-6-fluoro-5-methoxy-8-methyl-1H-quinazoline-2,4-dione;
7-(3-Aminomethylpyrrolidin-1-yl)-1-cyclopropyl-6-fluoro-5-methoxy-8-methyl-1H-quinazoline-2,4-dione;
7-[3-(1-Amino-2,2-difluoroethyl)pyrrolidin-1-yl]-1-cyclopropyl-6-fluoro-5-methoxy-8-methyl-1H-quinazoline-2,4-dione;
7-[3-(1-Amino-2,2,2-trifluoroethyl)pyrrolidin-1-yl]-1-cyclopropyl-6-fluoro-5-methoxy-8-methyl-1H-quinazoline-2,4-dione;
7-[3-(1-Aminoethyl)-4-fluoropyrrolidin-1-yl]-1-cyclopropyl-6-fluoro-5-methoxy-8-methyl-1H-quinazoline-2,4-dione;
1-Cyclopropyl-7-{3-[(2,6-difluorophenyl)hydroxymethyl]pyrrolidin-1-yl}-6-fluoro-5-methoxy-8-methyl-1H-quinazoline-2,4-dione;
7-(3-Aminomethyl-4-fluoromethylpyrrolidin-1-yl)-1-cyclopropyl-6-fluoro-5-methoxy-8-methyl-1H-quinazoline-2,4-dione;
7-[3-(Aminothiazol-2-ylmethyl)pyrrolidin-1-yl]-1-cyclopropyl-6-fluoro-5-methoxy-8-methyl-1H-quinazoline-2,4-dione;
1-Cyclopropyl-6-fluoro-7-[3-(1-hydroxycyclopropyl)pyrrolidin-1-yl]-5-methoxy-8-methyl-1H-quinazoline-2,4-dione;
7-[3-(Aminooxazol-4-ylmethyl)pyrrolidin-1-yl]-1-cyclopropyl-6-fluoro-5-methoxy-8-methyl-1H-quinazoline-2,4-dione;
7-(4-Amino-5,5-difluorooctahydroisoindol-2-yl)-1-cyclopropyl-5-difluoromethyl-6-fluoro-8-methyl-1H-quinazoline-2,4-dione;
7-(4-Amino-5,5-difluorohexahydrocyclopenta[c]pyrrol-2-yl)-1-cyclopropyl-5-difluoromethyl-6-fluoro-8-methyl-1H-quinazoline-2,4-dione;
7-(5,5-Difluoro-4-hydroxyoctahydroisoindol-2-yl)-1-cyclopropyl-5-difluoromethyl-6-fluoro-8-methyl-1H-quinazoline-2,4-dione;
7-(5,5-Difluoro-4-hydroxyhexahydrocyclopenta[c]pyrrol-2-yl)-1-cyclopropyl-5-difluoromethyl-6-fluoro-8-methyl-1H-quinazoline-2,4-dione;
7-[3-(1-Amino-2-hydroxyethyl)pyrrolidin-1-yl]-1-cyclopropyl-5-difluoromethyl-6-fluoro-8-methyl-1H-quinazoline-2,4-dione;
7-[3-(1-Amino-3,3,3-trifluoro-2-trifluoromethylpropyl)pyrrolidin-1-yl]-1-cyclopropyl-5-difluoromethyl-6-fluoro-8-methyl-1H-quinazoline-2,4-dione;
7-(4-Aminomethyl-4,5,6,7-tetrahydrobenzo[b]thiophen-2-yl)-1-cyclopropyl-5-difluoromethyl-6-fluoro-8-methyl-1H-quinazoline-2,4-dione;
1-Cyclopropyl-5-difluoromethyl-6-fluoro-7-(4-hydroxymethyl-4,5,6,7-tetrahydro-benzo[b]thiophen-2-yl)-8-methyl-1H-quinazoline-2,4-dione;
7-[4-(1-Aminoethyl)-4,5,6,7-tetrahydrobenzo[b]thiophen-2-yl]-1-cyclopropyl-5-difluoromethyl-6-fluoro-8-methyl-1H-quinazoline-2,4-dione;
7-[4-(2-Amino-1-hydroxyethyl)-4,5,6,7-tetrahydrobenzo[b]thiophen-2-yl]-201-cyclopropyl-5-difluoromethyl-6-fluoro-8-methyl-1H-quinazoline-2,4-dione;
7-[4-(1-Amino-2-hydroxyethyl)-4,5,6,7-tetrahydrobenzo[b]thiophen-2-yl]-1-cyclopropyl-5-difluoromethyl-6-fluoro-8-methyl-1H-quinazoline-2,4-dione;
7-[4-(1-Amino-2,2,2-trifluoroethyl)-4,5,6,7-tetrahydrobenzo[b]thiophen-2-yl]-1-cyclopropyl-5-difluoromethyl-6-fluoro-8-methyl-1H-quinazoline-2,4-dione;
7-(5-Aminomethyl-4,5,6,7-tetrahydrobenzo[b]thiophen-2-yl)-1-cyclopropyl-5-difluoromethyl-6-fluoro-8-methyl-1H-quinazoline-2,4-dione;
1-Cyclopropyl-5-difluoromethyl-6-fluoro-7-(5-hydroxymethyl-4,5,6,7-tetrahydro-benzo[b]thiophen-2-yl)-8-methyl-1H-quinazoline-2,4-dione;
7-[5-(1-Amino-2,2,2-trifluoroethyl)-4,5,6,7-tetrahydrobenzo[b]thiophen-2-yl]-1-cyclopropyl-5-difluoromethyl-6-fluoro-8-methyl-1H-quinazoline-2,4-dione;
7-[5-(1-Aminoethyl)-4,5,6,7-tetrahydrobenzo[b]thiophen-2-yl]-1-cyclopropyl-5-difluoromethyl-6-fluoro-8-methyl-1H-quinazoline-2,4-dione;
7-[5-(1-Amino-2-hydroxyethyl)-4,5,6,7-tetrahydrobenzo[b]thiophen-2-yl]-1-cyclopropyl-5-difluoromethyl-6-fluoro-8-methyl-1H-quinazoline-2,4-dione;
7-(3-Aminomethyl-4-trifluoromethylpyrrolidin-1-yl)-1-cyclopropyl-5-difluoromethyl-6-fluoro-8-methyl-1H-quinazoline-2,4-dione;
7-[3-(1-Amino-2,2,3,3,3-pentafluoropropyl)pyrrolidin-1-yl]-1-cyclopropyl-5-difluoromethyl-6-fluoro-8-methyl-1H-quinazoline-2,4-dione;
7-[3-(1-Amino-3,3,3-trifluoropropyl)pyrrolidin-1-yl]-1-cyclopropyl-5-difluoromethyl-6-fluoro-8-methyl-1H-quinazoline-2,4-dione;
7-[3-(1-Amino-3,3,3-trifluoro-2-trifluoromethylpropyl)pyrrolidin-1-yl]-1-cyclopropyl-5-difluoromethyl-6-fluoro-8-methyl-1H-quinazoline-2,4-dione
7-[4-(1-Aminoethyl)-3,3-difluoropyrrolidin-1-yl]-1-cyclopropyl-5-difluoromethyl-6-fluoro-8-methyl-1H-quinazoline-2,4-dione;
7-(3-Amino-4-ethylpiperidin-1-yl)-1-cyclopropyl-5-difluoromethyl-6-fluoro-8-methyl-1H-quinazoline-2,4-dione;
7-[3-(1-Amino-2-trifluoromethoxyethyl)pyrrolidin-1-yl]-1-cyclopropyl-5-difluoromethyl-6-fluoro-8-methyl-1H-quinazoline-2,4-dione;
7-(3-Aminomethyl-4-trifluoromethoxypyrrolidin-1-yl)-1-cyclopropyl-5-difluoromethyl-6-fluoro-8-methyl-1H-quinazoline-2,4-dione;
7-[3-Aminomethyl-4-(2,2,2-trifluoroethyl)pyrrolidin-1-yl]-1-cyclopropyl-5-difluoro-methyl-6-fluoro-8-methyl-1H-quinazoline-2,4-dione
7-[3-(1-Amino-2-difluoromethyl-3,3-difluoropropyl)pyrrolidin-1-yl]-1-cyclopropyl-5-difluoromethyl-6-fluoro-8-methyl-1H-quinazoline-2,4-dione;
7-[3-(-Amino-3-fluoro-2-fluoromethylpropyl)pyrrolidin-1-yl]-1-cyclopropyl-5-difluoromethyl-6-fluoro-8-methyl-1H-quinazoline-2,4-dione;
7-[3-(1-Amino-3,3-difluoropropyl)pyrrolidin-1-yl]-1-cyclopropyl-5-difluoromethyl-6-fluoro-8-methyl-1H-quinazoline-2,4-dione;
1-Cyclopropyl-7-(3,3-difluoro-4-hydroxymethylpyrrolidin-1-yl)-5-difluoromethyl-6-fluoro-8-methyl-1H-quinazoline-2,4-dione;
1-Cyclopropyl-5-difluoromethyl-7-[7-(1,2-dihydroxyethyl)-5-azaspiro[2.4]hept-5-yl]-6-fluoro-8-methyl-1H-quinazoline-2,4-dione;
1-Cyclopropyl-7-(5,5-difluoro-4-hydroxymethyl-4,5,6,7-tetrahydrobenzo[b]thiophen-2-yl)-5-difluoromethyl-6-fluoro-8-methyl-1H-quinazoline-2,4-dione;
7-[3-(1-Amino-2,2,2-trifluoro-1-trifluoromethylethyl)pyrrolidin-1-yl-1-cyclopropyl-5-difluoromethyl-6-fluoro-8-methyl-1H-quinazoline-2,4-dione;
7-[3-Aminomethyl-4-(3,3,3-trifluoro-2-trifluoromethylpropyl)pyffolidin-1-yl]-1-cyclopropyl-difluoromethyl-6-fluoro-8-methyl-1H-quinazoline-2,4-dione;
7-[3-(1-Amino-4,4,4-trifluoro-3-trifluoromethylbut-2-enyl)pyrrolidin-1-yl]-1-cyclopropyl-5-difluoromethyl-6-fluoro-8-methyl-1H-quinazoline-2,4-dione;
7-[3-(1-Amino-4,4,4-trifluoro-3-trifluoromethylbutyl)pyrrolidin-1-yl]-1-cyclopropyl-5-difluoromethyl-6-fluoro-8-methyl-1H-quinazoline-2,4-dione;
7-[3-(1-Amino-2-hydroxyethyl)-4-fluoropyrrolidin-1-yl-1-cyclopropyl-5-difluoromethyl-6-fluoro-8-methyl-1H-quinazoline-2,4-dione;
7-[3-(1-Amino-2,2,2-trifluoroethyl)-4-fluoropyrrolidin-1-yl]-1-cyclopropyl-5-difluoromethyl-6-fluoro-8-methyl-1H-quinazoline-2,4-dione;
7-(3-Aminomethyl-4-difluoromethoxypyrrolidin-1-yl)-1-cyclopropyl-5-difluoromethyl-6-fluoro-8-methyl-1H-quinazoline-2,4-dione;
7-(4-Aminomethyl-5,5-difluoro-4,5,6,7-tetrahydrobenzo[b]thiophen-2-yl)-1-cyclopropyl-difluoromethyl-6-fluoro-8-methyl-1H-quinazoline-2,4-dione;
7-(4-Amino-5,5-difluorooctahydrocyclohepta[c]pyrrol-2-yl)-1-cyclopropyl-5-difluoromethyl-6-fluoro-8-methyl-1H-quinazoline-2,4-dione;
1-Cyclopropyl-7-(5,5-difluoro-4-hydroxyoctahydrocyclohepta[c]pyrrol-2-yl)-5-difluoromethyl-6-fluoro-8-methyl-1H-quinazoline-2,4-dione;
7-[5-(1-Aminoethyl)thiophen-3-yl]-1-cyclopropyl-5-difluoromethyl-6-fluoro-8-methyl-1H-quinazoline-2,4-dione;
7-(5-Aminomethylthiophen-3-yl)-1-cyclopropyl-5-difluoromethyl-6-fluoro-8-methyl-1H-quinazoline-2,4-dione;
7-(4-Amino-5,5-difluoro-4,5,6,7-tetrahydrobenzo[b]thiophen-2-yl)-1-cyclopropyl-5-difluoromethyl-6-fluoro-8-methyl-1H-quinazoline-2,4-dione;
1-Cyclopropyl-7-(5,5-difluoro-4-hydroxy-4,5,6,7-tetrahydrobenzo[b]thiophen-2-yl)-5-difluoromethyl-6-fluoro-8-methyl-1H-quinazoline-2,4-dione;
7-(4-Amino-5,5-difluorooctahydroisoindol-2-yl)-1-cyclopropyl-5-difluoromethyl-6-fluoro-8-methoxy-1H-quinazoline-2,4-dione;
7-(4-Amino-5,5-difluorohexahydrocyclopenta[c]pyrrol-2-yl)-1-cyclopropyl-5-difluoro-methyl-6-fluoro-8-methoxy-1H-quinazoline-2,4-dione;
1-Cyclopropyl-7-(5,5-difluoro-4-hydroxyhexahydrocyclopenta[c]pyrrol-2-yl)-5-difluoro-methyl-6-fluoro-8-methoxy-1H-quinazoline-2,4-dione;
7-[3-(1-Amino-2-hydroxyethyl)pyrrolidin-1-yl]-1-cyclopropyl-5-difluoromethyl-6-fluoro-8-methoxy-1H-quinazoline-2,4-dione;
1-Cyclopropyl-7-(5,5-difluoro-4-hydroxyoctahydroisoindol-2-yl)-5-difluoromethyl-6-fluoro-8-methoxy-1H-quinazoline-2,4-dione;
7-[3-(1-Amino-2-hydroxyethyl)pyrrolidin-1-yl]-1-cyclopropyl-5-difluoromethyl-6-fluoro-8-methoxy-1H-quinazoline-2,4-dione;
7-[3-(1-Amino-3,3,3-trifluoro-2-trifluoromethylpropyl)pyrrolidin-1-yl]-1-cyclopropyl-5-difluoromethyl-6-fluoro-8-methoxy-1H-quinazoline-2,4-dione;
7-(4-Aminomethyl-4,5,6,7-tetrahydrobenzo[b]thiophen-2-yl)-1-cyclopropyl-5-difluoromethyl-6-fluoro-8-methoxy-1H-quinazoline-2,4-dione;
1-Cyclopropyl-5-difluoromethyl-6-fluoro-7-(4-hydroxymethyl-4,5,6,7-tetrahydro-benzo[b]thiophen-2-yl)-8-methooxy-1H-quinazoline-2,4-dione;
7-[4-(1-Aminoethyl)-4,5,6,7-tetrahydrobenzo[b]thiophen-2-yl]-1-cyclopropyl-5-difluoromethyl-6-fluoro-8-methoxy-1H-quinazoline-2,4-dione;
7-[4-(2-Amino-1-hydroxyethyl)-4,5,6,7-tetrahydrobenzo[b]thiophen-2-yl]-1-cyclopropyl-5-difluoromethyl-6-fluoro-8-methoxy-1H-quinazoline-2,4-dione;
7-[4-(1-Amino-2-hydroxyethyl)-4,5,6,7-tetrahydrobenzo[b]thiophen-2-y]-1-cyclopropyl-5-difluoromethyl-6-fluoro-8-methoxy-1H-quinazoline-2,4-dione;
7-[4-(-Amino-2,2,2-trifluoroethyl)-4,5,6,7-tetrahydrobenzo[b]thiophen-2-yl]-1-cyclopropyl-5-difluoromethyl-6-fluoro-8-methoxy-1H-quinazoline-2,4-dione
7-(5-Aminomethyl-4,5,6,7-tetrahydrobenzo[b]thiophen-2-yl)-1-cyclopropyl-5-difluoromethyl-6-fluoro-8-methoxy-1H-quinazoline-2,4-dione;
1-Cyclopropyl-5-difluoromethyl-6-fluoro-7-(5-hydroxymethyl-4,5,6,7-tetrahydro-benzo[b]thiophen-2-yl)-8-methoxy-1H-quinazoline-2,4-dione;
7-[5-(1-Amino-2,2,2-trifluoroethyl)-4,5,6,7-tetrahydrobenzo[b]thiophen-2-yl]-1-cyclopropyl-5-difluoromethyl-6-fluoro-8-methoxy-1H-quinazoline-2,4-dione
7-[5-(1-Aminoethyl)-4,5,6,7-tetrahydrobenzo[b]thiophen-2-yl]-1-cyclopropyl-5-difluoromethyl-6-fluoro-8-methoxy-1H-quinazoline-2,4-dione;
7-[5-(1-Amino-2-hydroxyethyl)-4,5,6,7-tetrahydrobenzo[b]thiophen-2-yl]-1-cyclopropyl-5-difluoromethyl-6-fluoro-8-methoxy-1H-quinazoline-2,4-dione;
7-[5-(2-Amino-1-hydroxyethyl)-4,5,6,7-tetrahydrobenzo[b]thiophen-2-yl]-1-cyclopropyl-5-difluoromethyl-6-fluoro-8-methoxy-1H-quinazoline-2,4-dione;
7-(3-Aminomethyl-4-trifluoromethylpyrrolidin-1-yl)-1-cyclopropyl-5-difluoromethyl-6-fluoro-8-methoxy-1H-quinazoline-2,4-dione;
7-[3-(1-Amino-2,2,3,3,3-pentafluoropropyl)pyrrolidin-1-yl]-1-cyclopropyl-5-difluoromethyl-6-fluoro-8-methoxy-1H-quinazoline-2,4-dione;
7-[4-(1-Aminoethyl)-3,3-difluoropyrrolidin-1-yl]-1-cyclopropyl-5-difluoromethyl-6-fluoro-8-methoxy-1H-quinazoline-2,4-dione;
7-(3-Amino-4-ethylpiperidin-1-yl)-1-cyclopropyl-5-difluoromethyl-6-fluoro-8-methoxy-1H-quinazoline-2,4-dione;
7-[3-(1-Amino-2-trifluoromethoxyethyl)pyrrolidin-1-yl]-1-cyclopropyl-5-difluoromethyl-6-fluoro-8-methoxy-1H-quinazoline-2,4-dione;
7-(3-Aminomethyl-4-trifluoromethoxypyrrolidin-1-yl)-1-cyclopropyl-5-difluoromethyl-6-fluoro-8-methoxy-1H-quinazoline-2,4-dione;
7-[3-Aminomethyl-4-(2,2,2-trifluoroethyl)pyrrolidin-1-yl]-1-cyclopropyl-5-difluoro-methyl-6-fluoro-8-methoxy-1H-quinazoline-2,4-dione;
7-[3-(1-Amino-2-difluoromethyl-3,3-difluoropropyl)pyrrolidin-1-yl]-1-cyclopropyl-5-difluoromethyl-6-fluoro-8-methoxy-1H-quinazoline-2,4-dione;
7-[3-(1-Amino-3-fluoro-2-fluoromethylpropyl)pyrrolidin-1-yl]-1-cyclopropyl-5-difluoromethyl-6-fluoro-8-methoxy-1H-quinazoline-2,4-dione;
7-[3-(1-Amino-3,3-difluoropropyl)pyrrolidin-1-yl]-1-cyclopropyl-5-difluoromethyl-6-fluoro-8-methoxy-1H-quinazoline-2,4-dione;
1-Cyclopropyl-7-(3,3-difluoro-4-hydroxymethylpyrrolidin-1-yl)-5-difluoromethyl-6-fluoro-8-methoxy-1H-quinazoline-2,4-dione;
1-Cyclopropyl-5-difluoromethyl-7-[7-(1,2-dihydroxyethyl)-5-azaspiro[2.4]hept-5-yl]-6-fluoro-8-methoxy-1H-quinazoline-2,4-dione;
1-Cyclopropyl-7-(5,5-difluoro-4-hydroxymethyl-4,5,6,7-tetrahydrobenzo[b]thiophen-2-yl)-5-difluoromethyl-6-fluoro-8-methoxy-1H-quinazoline-2,4-dione;
7-[3-(1-Amino-2,2,2-trifluoro-1-trifluoromethylethyl)pyrrolidin-1-yl]-1-cyclopropyl-5-difluoromethyl-6-fluoro-8-methoxy-1H-quinazoline-2,4-dione;
7-[3-Aminomethyl-4-(3,3,3-trifluoro-2-trifluoromethylpropyl)pyrrolidin-1-yl]-1-cyclopropyl-5-difluoromethyl-6-fluoro-8-methoxy-1H-quinazoline-2,4-dione;
7-[3-(1-Amino-4,4,4-trifluoro-3-trifluoromethylbut-2-enyl)pyrrolidin-1-yl]-1-cyclopropyl-5-difluoromethyl-6-fluoro-8-methoxy-1H-quinazoline-2,4-dione;
7-[3-(1-Amino-4,4,4-trifluoro-3-trifluoromethylbutyl)pyrrolidin-1-yl]-1-cyclopropyl-5-difluoromethyl-6-fluoro-8-methoxy-1H-quinazoline-2,4-dione;
7-[3-(1-Amino-2-hydroxyethyl)-4-fluoropyrrolidin-1-yl]-1-cyclopropyl-5-difluoromethyl-6-fluoro-8-methoxy-1H-quinazoline-2,4-dione;
7-[3-(1-Amino-2,2,2-trifluoroethyl)-4-fluoropyrrolidin-1-yl]-1-cyclopropyl-5-difluoromethyl-6-fluoro-8-methoxy-1H-quinazoline-2,4-dione;
7-(3-Aminomethyl-4-difluoromethoxypyrrolidin-1-yl)-1-cyclopropyl-5-difluoromethyl-6-fluoro-8-methoxy-1H-quinazoline-2,4-dione;
7-(4-Aminomethyl-5,5-difluoro-4,5,6,7-tetrahydrobenzo[b]thiophen-2-yl)-1-cyclopropyl-5-difluoromethyl-6-fluoro-8-methoxy-1H-quinazoline-2,4-dione;
7-(4-Amino-5,5-difluorooctahydrocyclohepta[c]pyrrol-2-yl)-1-cyclopropyl-5-difluoromethyl-6-fluoro-8-methoxy-1H-quinazoline-2,4-dione;
1-Cyclopropyl-7-(5,5-difluoro-4-hydroxyoctahydrocyclohepta[c]pyrrol-2-yl)-5-difluoromethyl-6-fluoro-8-methoxy-1H-quinazoline-2,4-dione;
7-[5-(1-Aminoethyl)thiophen-3-yl]-]-cyclopropyl-5-difluoromethyl-6-fluoro-8-methoxy-1H-quinazoline-2,4-dione;
7-(5-Aminomethylthiophen-3-yl)-1-cyclopropyl-5-difluoromethyl-6-fluoro-8-methoxy-1H-quinazoline-2,4-dione;
7-(4-Amino-5,5-difluoro-4,5,6,7-tetrahydrobenzo[b]thiophen-2-yl)-1-cyclopropyl-5-difluoromethyl-6-fluoro-8-methoxy-1H-quinazoline-2,4-dione;
1-Cyclopropyl-7-(5,5-difluoro-4-hydroxy-4,5,6,7-tetrahydrobenzo[b]thiophen-2-yl)-5,8-dimethyl-6-fluoro-1H-quinazoline-2,4-dione;
7-(4-Amino-5,5-difluorooctahydroisoindol-2-yl)-1-cyclopropyl-5,8-dimethyl-6-fluoro-1H-quinazoline-2,4-dione;
7-(4-Amino-5,5-difluorohexahydrocyclopenta[c]pyrrol-2-yl)-1-cyclopropyl-5,8-dimethyl-6-fluoro-1H-quinazoline-2,4-dione;
7-(5,5-Difluoro-4-hydroxyoctahydroisoindol-2-yl)-1-cyclopropyl-5,8-dimethyl-6-fluoro-1H-quinazoline-2,4-dione;
7-(5,5-Difluoro-4-hydroxyhexahydrocyclopenta[c]pyrrol-2-yl)-1-cyclopropyl-5,8-dimethyl-6-fluoro-1H-quinazoline-2,4-dione;
7-[3-(1-Amino-2-hydroxyethyl)pyrrolidin-1-yl]-1-cyclopropyl-5-dimethyl-6-fluoro-1H-quinazoline-2,4-dione;
7-[3-(1-Amino-3,3,3-trifluoro-2-trifluoromethylpropyl)pyrrolidin-1-yl]-1-cyclopropyl-5,8-dimethyl-6-fluoro-]1H-quinazoline-2,4-dione;
7-(4-Aminomethyl-4,5,6,7-tetrahydrobenzo[b]thiophen-2-yl)-1-cyclopropyl-5,8-dimethyl-6-fluoro-1H-quinazoline-2,4-dione;
1-Cyclopropyl-5,8-dimethyl-6-fluoro-7-(4-hydroxymethyl-4,5,6,7-tetrahydro-benzo[b]thiophen-2-yl)-1H-quinazoline-2,4-dione;
7-[4-(1-Aminoethyl)-4,5,6,7-tetrahydrobenzo[b]thiophen-2-yl]-1-cyclopropyl-5,8-dimethyl-6-fluoro-1H-quinazoline-2,4-dione;
7-[4-(2-Amino-1-hydroxyethyl)-4,5,6,7-tetrahydrobenzo[b]thiophen-2-yl]-1-cyclopropyl-5,8-dimethyl-6-fluoro-1H-quinazoline-2,4-dione;
7-[4-(1-Amino-2-hydroxyethyl)-4,5,6,7-tetrahydrobenzo[b]thiophen-2-yl]-1-cyclopropyl-5,8-dimethyl-6-fluoro-1H-quinazoline-2,4-dione;
7-[4-(1-Amino-2,2,2-trifluoroethyl)-4,5,6,7-tetrahydrobenzo[b]thiophen-2-yl]-1-cyclopropyl-5,8-dimethyl-6-fluoro-1H-quinazoline-2,4-dione;
7-(5-Aminomethyl-4,5,6,7-tetrahydrobenzo[b]thiophen-2-yl)-1-cyclopropyl-5,8-dimethyl-6-fluoro-1H-quinazoline-2,4-dione;
1-Cyclopropyl-5,8-dimethyl-6-fluoro-7-(5-hydroxymethyl-4,5,6,7-tetrahydro-benzo[b]thiophen-2-yl)-1H-quinazoline-2,4-dione;
7-[5-(1-Amino-2,2,2-trifluoroethyl)-4,5,6,7-tetrahydrobenzo[b]thiophen-2-yl]-1-cyclopropyl-5,8-methyl-6-fluoro-1H-quinazoline-2,4-dione;
7-(3-Aminomethyl-4-trifluoromethylpyrrolidin-1-yl)-1-cyclopropyl-5,8-dimethyl-6-fluoro-1H-quinazoline-2,4-dione;
7-[3-(1-Amino-2,2,3,3,3-pentafluoropropyl)pyrrolidin-1-yl]-1-cyclopropyl-5,8-dimethyl-6-fluoro-1H-quinazoline-2,4-dione;
7-[3-(1-Amino-3,3,3-trifluoropropyl)pyrrolidin-1-yl]-1-cyclopropyl-5,8-dimethyl-6-fluoro-1H-quinazoline-2,4-dione;
7-[3-(1-Amino-3,3,3-trifluoro-2-trifluoromethylpropyl)pyrrolidin-1-yl]-1-cyclopropyl-5,8-dimethyl-6-fluoro-1H-quinazoline-2,4-dione;
7-(3-Aminomethyl-4-trifluoromethylpyrrolidin-1-yl)-1-cyclopropyl-5,8-dimethyl-6-fluoro-1H-quinazoline-2,4-dione;
7-[3-(1-Amino-2,2,3,3,3-pentafluoropropyl)pyrrolidin-1-yl]-1-cyclopropyl-5,8-dimethyl-6-fluoro-1H-quinazoline-2,4-dione;
7-[3-(1-Amino-3,3,3-trifluoropropyl)pyrrolidin-1-yl]-1-cyclopropyl-5,8-dimethyl-6-fluoro-1H-quinazoline-2,4-dione;
7-[4-(1-Aminoethyl)-3,3-difluoropyrrolidin-1-yl]-1-cyclopropyl-5,8-dimethyl-6-fluoro-1H-quinazoline-2,4-dione;
7-(3-Amino-4-ethylpiperidin-1-yl)-1-cyclopropyl-5,8-dimethyl-6-fluoro-8-1H-quinazoline-2,4-dione;
7-[3-(1-Amino-2-trifluoromethoxyethyl)pyrrolidin-1-yl]-1-cyclopropyl-5,8-dimethyl-6-fluoro-1H-quinazoline-2,4-dione;
7-(3-Aminomethyl-4-trifluoromethoxypyrrolidin-1-yl)-1-cyclopropyl-5,8-dimethyl-6-fluoro-1H-quinazoline-2,4-dione;
7-[3-Aminomethyl-4-(2,2,2-trifluoroethyl)pyrrolidin-1-yl]-1-cyclopropyl-5,8-dimethyl-6-fluoro-1H-quinazoline-2,4-dione
7-[3-(1-Amino-2,2-difluoromethyl-3,3-difluoropropyl)pyrrolidin-1-yl]-1-cyclopropyl-5,8-dimethyl-6-fluoro-1H-quinazoline-2,4-dione;
7-[3-(1-Amino-3-fluoro-2-fluoromethylpropyl)pyrrolidin-1-yl]-1-cyclopropyl-5,8-dimethyl-6-fluoro-1H-quinazoline-2,4-dione;
7-[3-(1-Amino-3,3-difluoropropyl)pyrrolidin-1-yl]-1-cyclopropyl-5,8-dimethyl-6-fluoro-1H-quinazoline-2,4-dione;
1-Cyclopropyl-7-(3,3-difluoro-4-hydroxymethylpyrrolidin-1-yl)-5,8-dimethyl-6-fluoro-1H-quinazoline-2,4-dione;
1-Cyclopropyl-5,8-dimethyl-7-[7-(1,2-dihydroxyethyl)-5-azaspiro[2.4]hept-5-yl]-6-fluoro-1H-quinazoline-2,4-dione;
1-Cyclopropyl-7-(5,5-difluoro-4-hydroxymethyl-4,5,6,7-tetrahydrobenzo[b]thiophen-2-yl)-5,8-dimethyl-6-fluoro-1H-quinazoline-2,4-dione;
7-[3-(1-Amino-2,2,2-trifluoro-1,1,1-trifluoromethylethyl)pyrrolidin-1-yl]-1-cyclopropyl-5,8-dimethyl-6-fluoro-1H-quinazoline-2,4-dione;
7-[3-Aminomethyl-4-(3,3,3-trifluoro-2,2,2-trifluoromethylpropyl)pyrrolidin-1-yl]-1-cyclopropyl-5,8-dimethyl-6-fluoro-1H-quinazoline-2,4-dione;
7-[3-(1-Amino-4,4,4-trifluoro-3,3,-trifluoromethylbut-2-enyl)pyrrolidin-1-yl]-1-cyclopropyl-5,8-dimethyl-6-fluoro-1H-quinazoline-2,4-dione;
7-[3-(1-Amino-4,4,4-trifluoro-3,3,3-trifluoromethylbutyl)pyrrolidin-1-yl]-1-cyclopropyl-5,8-dimethyl-6-fluoro-1H-quinazoline-2,4-dione;
7-[3-(1-Amino-2-hydroxyethyl)-4-fluoropyrrolidin-1-yl]-1-cyclopropyl-5,8-dimethyl-6-fluoro-1H-quinazoline-2,4-dione;
7-[3-(1-Amino-2,2,2-trifluoroethyl)-4-fluoropyrrolidin-1-yl]-1-cyclopropyl-5,8-dimethyl-6-fluoro-1H-quinazoline-2,4-dione;
7-(3-Aminomethyl-4,4-difluoromethoxypyrrolidin-1-yl)-1-cyclopropyl-5,8-dimethyl-6-fluoro-1H-quinazoline-2,4-dione;
7-(4-Aminomethyl-5,5-difluoro-4,5,6,7-tetrahydrobenzo[b]thiophen-2-yl)-1-cyclopropyl-5,8-dimethyl-6-fluoro-1H-quinazoline-2,4-dione;
7-(4-Amino-5,5-difluorooctahydrocyclohepta[c]pyrrol-2-yl)-1-cyclopropyl-5,8-dimethyl-6-fluoro-1H-quinazoline-2,4-dione;
1-Cyclopropyl-7-(5,5-difluoro-4-hydroxyoctahydrocyclohepta[c]pyrrol-2-yl)-5,8-dimethyl-6-fluoro-1H-quinazoline-2,4-dione;
7-[5-(1-Aminoethyl)thiophen-3-yl]-1-cyclopropyl-5,8-dimethyl-6-fluoro-1H-quinazoline-2,4-dione;
7-(5-Aminomethylthiophen-3-yl)-1-cyclopropyl-5,8-dimethyl-6-fluoro-1H-quinazoline-2,4-dione;
7-(4-Amino-5,5-difluoro-4,5,6,7-tetrahydrobenzo[b]thiophen-2-yl)-1-cyclopropyl-5,8-dimethyl-6-fluoro-1H-quinazoline-2,4-dione;
1-Cyclopropyl-7-(5,5-difluoro-4-hydroxy-4,5,6,7-tetrahydrobenzo[b]thiophen-2-yl)-5,8-dimethyl-6-fluoro-1H-quinazoline-2,4-dione;
7-(4-Amino-5,5-difluorooctahydroisoindol-2-yl)-1-cyclopropyl-5-methyl-6-fluoro-8-methoxy-1H-quinazoline-2,4-dione;
7-(4-Amino-5,5-difluorohexahydrocyclopenta[c]pyrrol-2-yl)-1-cyclopropyl-5-methyl-6-fluoro-8-methoxy-1H-quinazoline-2,4-dione;
7-(5,5-Difluoro-4-hydroxyoctahydroisoindol-2-yl)-1-cyclopropyl-5-methyl-6-fluoro-8-methoxy-1H-quinazoline-2,4-dione;
7-(5,5-Difluoro-4-hydroxyhexahydrocyclopenta[c]pyrrol-2-yl)-1-cyclopropyl-5-methyl-6-fluoro-8-methoxy-1H-quinazoline-2,4-dione;
7-[3-(1-Amino-2-hydroxyethyl)pyrrolidin-1-yl]-1-cyclopropyl-5-methyl-6-fluoro-8-methoxy-1H-quinazoline-2,4-dione;
7-[3-(1-Amino-3,3,3-trifluoro-2,2,2-trifluoromethylpropyl)pyrrolidin-1-yl]-1-cyclopropyl-5-methyl-6-fluoro-8-methoxy-1H-quinazoline-2,4-dione;
7-(4-Aminomethyl-4,5,6,7-tetrahydrobenzo[b]thiophen-2-yl)-1-cyclopropyl-5-methyl-6-fluoro-8-methoxy-1H-quinazoline-2,4-dione;
1-Cyclopropyl-5-methyl-6-fluoro-7-(4-hydroxymethyl-4,5,6,7-tetrahydro-benzo[b]thiophen-2-yl)-8-methoxy-1H-quinazoline-2,4-dione;
7-[4-(1-Aminoethyl)-4,5,6,7-tetrahydrobenzo[b]thiophen-2-yl]-1-cyclopropyl-5-methyl-6-fluoro-8-methoxy-1H-quinazoline-2,4-dione;
7-[4-(2-Amino-1-hydroxyethyl)-4,5,6,7-tetrahydrobenzo[b]thiophen-2-yl]-1-cyclopropyl-5-methyl-6-fluoro-8-methoxy-1H-quinazoline-2,4-dione;
7-[4-(1-Amino-2-hydroxyethyl)-4,5,6,7-tetrahydrobenzo[b]thiophen-2-yl]-1-cyclopropyl-5-methyl-6-fluoro-8-methoxy-1H-quinazoline-2,4-dione;
7-[4-(1-Amino-2,2,2-trifluoroethyl)-4,5,6,7-tetrahydrobenzo[b]thiophen-2-yl]-1-cyclopropyl-5-methyl-6-fluoro-8-methxoy-1H-quinazoline-2,4-dione;
7-(5-Aminomethyl-4,5,6,7-tetrahydrobenzo[b]thiophen-2-yl)-1-cyclopropyl-5-methyl-6-fluoro-8-methoxy-1H-quinazoline-2,4-dione;
1-Cyclopropyl-5-methyl-6-fluoro-7-(5-hydroxymethyl-4,5,6,7-tetrahydro-benzo[b]thiophen-2-yl)-8-methoxy-1H-quinazoline-2,4-dione;
7-[5-(1-Amino-2,2,2-trifluoroethyl)-4,5,6,7-tetrahydrobenzo[b]thiophen-2-yl]-1-cyclopropyl-5-methyl-6-fluoro-8-methoxy-1H-quinazoline-2,4-dione;
7-(3-Aminomethyl-4-trifluoromethylpyrrolidin-1-yl)-1-cyclopropyl-5-methyl-6-fluoro-8-methoxy-1H-quinazoline-2,4-dione;
7-[3-(1-Amino-2,2,3,3,3-pentafluoropropyl)pyrrolidin-1-yl]-1-cyclopropyl-5-methyl-6-fluoro-8-methoxy-1H-quinazoline-2,4-dione;
7-[3-(1-Amino-3,3,3-trifluoropropyl)pyrrolidin-1-yl]-1-cyclopropyl-5-methyl-6-fluoro-8-methoxy-1H-quinazoline-2,4-dione;
7-[3-(1-Amino-3,3,3-trifluoro-2,2,2-trifluoromethylpropyl)pyrrolidin-1-yl]-1-cyclopropyl-5-methyl-6-fluoro-8-methoxy-1H-quinazoline-2,4-dione;
7-(3-Aminomethyl-4,4,4-trifluoromethylpyrrolidin-1-yl)-1-cyclopropyl-5-methyl-6-fluoro-8-methoxy-1H-quinazoline-2,4-dione;
7-[3-(1-Amino-2,2,3,3,3-pentafluoropropyl)pyrrolidin-1-yl]-1-cyclopropyl-5-methyl-6-fluoro-8-methoxy-1H-quinazoline-2,4-dione;
7-[3-(1-Amino-3,3,3-trifluoropropyl)pyrrolidin-1-yl]-1-cyclopropyl-5-methyl-6-fluoro-8-methoxy-1H-quinazoline-2,4-dione;
7-(3-Aminomethyl-4-trifluoromethylpyrrolidin-1-yl)-1-cyclopropyl-5-methyl-6-fluoro-8-methoxy-1H-quinazoline-2,4-dione;
7-[3-(1-Amino-2,2,3,3,3-pentafluoropropyl)pyrrolidin-1-yl]-1-cyclopropyl-5-methyl-6-fluoro-8-methoxy-1H-quinazoline-2,4-dione;
7-[3-(1-Amino-3,3,3-trifluoropropyl)pyrrolidin-1-yl-1-cyclopropyl-5-methyl-6-fluoro-8-methoxy-1H-quinazoline-2,4-dione;
7-[4-(1-Aminoethyl)-3,3-difluoropyrrolidin-1-yl]-1-cyclopropyl-5-methyl-6-fluoro-8-methoxy-1H-quinazoline-2,4-dione;
7-(3-Amino-4-ethylpiperidin-1-yl)-1-cyclopropyl-5-methyl-6-fluoro-8-methoxy-1H-quinazoline-2,4-dione;
7-[3-(1-Amino-2-trifluoromethoxyethyl)pyrrolidin-1-yl]-1-cyclopropyl-5-methyl-6-fluoro-8-methoxy-1H-quinazoline-2,4-dione;
7-(3-Aminomethyl-4,4,4-trifluoromethoxypyrrolidin-1-yl)-1-cyclopropyl-5-methyl-6-fluoro-8-methoxy-1H-quinazoline-2,4-dione;
7-[3-Aminomethyl-4-(2,2,2-trifluoroethyl)pyrrolidin-1-yl]-1-cyclopropyl-5-methyl-6-fluoro-8-methoxy-1H-quinazoline-2,4-dione;
7-[3-(1-Amino-2,2-difluoromethyl-3,3-difluoropropyl)pyrrolidin-1-yl]-1-cyclopropyl-5-methyl-6-fluoro-8-methoxy-1H-quinazoline-2,4-dione;
7-[3-(1-Amino-3-fluoro-2-fluoromethylpropyl)pyrrolidin-1-yl]-1-cyclopropyl-5-methyl-6-fluoro-8-methoxy-1H-quinazoline-2,4-dione;
7-[3-(1-Amino-3,3-difluoropropyl)pyrrolidin-1-yl]-1-cyclopropyl-5-methyl-6-fluoro-8-methoxy-1H-quinazoline-2,4-dione;
1-Cyclopropyl-7-(3,3-difluoro-4-hydroxymethylpyrrolidin-1-yl)-5-methyl-6-fluoro-8-methoxy-1H-quinazoline-2,4-dione;
1-Cyclopropyl-5-methyl-7-[7-(,2-dihydroxyethyl)-5-azaspiro[2.4]hept-5-yl]-6-fluoro-8-methoxy-1H-quinazoline-2,4-dione;
1-Cyclopropyl-7-(5,5-difluoro-4-hydroxymethyl-4,5,6,7-tetrahydrobenzo[b]thiophen-2-yl)-5-methyl-6-fluoro-8-methoxy-1H-quinazoline-2,4-dione;
7-[3-(1-Amino-2,2,2-trifluoro-1,1,1-trifluoromethylethyl)pyrrolidin-1-yl]-1-cyclopropyl-5-methyl-6-fluoro-8-methoxy-1H-quinazoline-2,4-dione;
7-[3-Aminomethyl-4-(3,3,3-trifluoro-2-trifluoromethylpropyl)pyrrolidin-1-yl]-1-cyclopropyl-5-methyl-6-fluoro-8-methoxy-1H-quinazoline-2,4-dione;
7-[3-(1-Amino-4,4,4-trifluoro-3,3,3-trifluoromethylbut-2-enyl)pyrrolidin-1-yl]-1-cyclopropyl-5-methyl-6-fluoro-8-methoxy-1H-quinazoline-2,4-dione;
7-[3-(1-Amino-4,4,4-trifluoro-3,3,3-trifluoromethylbutyl)pyrrolidin-1-yl]-1-cyclopropyl-5-methyl-6-fluoro-8-methoxy-1H-quinazoline-2,4-dione;
7-[3-(1-Amino-2-hydroxyethyl)-4-fluoropyrrolidin-1-yl]-1-cyclopropyl-5-methyl-6-fluoro-8-methoxy-1H-quinazoline-2,4-dione;
7-[3-(1-Amino-2,2,2-trifluoroethyl)-4-fluoropyrrolidin-1-yl]-1-cyclopropyl-5-methyl-6-fluoro-8-methoxy-1H-quinazoline-2,4-dione;
7-(3-Aminomethyl-4,4-difluoromethoxypyrrolidin-1-yl)-1-cyclopropyl-5-methyl-6-fluoro-8-methoxy-1H-quinazoline-2,4-dione;
7-(4-Aminomethy]-5,5-difluoro-4,5,6,7-tetrahydrobenzo[b]thiophen-2-yl)-1-cyclopropyl-5-methyl-6-fluoro-8-methoxy-1H-quinazoline-2,4-dione;
7-(4-Amino-5,5-difluorooctahydrocyclohepta[c]pyrrol-2-yl)-1-cyclopropyl-5-methyl-6-fluoro-8-methoxy-1H-quinazoline-2,4-dione;
1-Cyclopropyl-7-(5,5-difluoro-4-hydroxyoctahydrocyclohepta[c]pyrrol-2-yl)-5-methyl-6-fluoro-8-methoxy-1H-quinazoline-2,4-dione;
7-[5-(1-Aminoethyl)thiophen-3-yl]-1-cyclopropyl-5-methyl-6-fluoro-8-methoxy-1H-quinazoline-2,4-dione;
7-(5-Aminomethylthiophen-3-yl)-1-cyclopropyl-5-methyl-6-fluoro-8-methoxy-1H-quinazoline-2,4-dione;
7-(4-Amino-5,5-difluoro-4,5,6,7-tetrahydrobenzo[b]thiophen-2-yl)-1-cyclopropyl-5-methyl-6-fluoro-8-methoxy-1H-quinazoline-2,4-dione;
1-Cyclopropyl-7-(5,5-difluoro-4-hydroxy-4,5,6,7-tetrahydrobenzo[b]thiophen-2-yl)-5-methyl-6-fluoro-8-methoxy-1H-quinazoline-2,4-dione;
5-Amino-7-(4-amino-5,5-difluorooctahydroisoindol-2-yl)-1-cyclopropyl-6-fluoro-8-methyl-1H-quinazoline-2,4-dione;
5-Amino-7-(4-amino-5,5-difluorohexahydrocyclopenta[c]pyrrol-2-yl)-1-cyclopropyl-6-fluoro-8-methyl-1H-quinazoline-2,4-dione;
5-Amino-7-(5,5-difluoro-4-hydroxyoctahydroisoindol-2-yl)-1-cyclopropyl-6-fluoro-8-methyl-1H-quinazoline-2,4-dione;
5-Amino-7-(5,5-difluoro-4-hydroxyhexahydrocyclopenta[c]pyrrol-2-yl)-1-cyclopropyl-6-fluoro-8-methyl-1H-quinazoline-2,4-dione;
5-Amino-7-[3-(1-amino-2-hydroxyethyl)pyrrolidin-1-yl]-1-cyclopropyl-6-fluoro-8-methyl-1H-quinazoline-2,4-dione;
5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazoline-2,4-dione;
5-Amino-7-(4-aminomethyl-4,5,6,7-tetrahydrobenzo[b]thiophen-2-yl)-1-cyclopropyl-6-fluoro-8-methyl-1H-quinazoline-2,4-dione;
5-Amino-1-cyclopropyl-6-fluoro-7-(4-hydroxymethyl-4,5,6,7-tetrahydrobenzo[b]thiophen-2-yl)-8-methyl-1H-quinazoline-2,4-dione;
5-Amino-7-[4-(1-aminoethyl)-4,5,6,7-tetrahydrobenzo[b]thiophen-2-yl]-1-cyclopropyl-6-fluoro-8-methyl-1H-quinazoline-2,4-dione;
5-Amino-7-[4-(2-amino-1-hydroxyethyl)-4,5,6,7-tetrahydrobenzo[b]thiophen-2-yl]-1-cyclopropyl-6-fluoro-8-methyl-1H-quinazoline-2,4-dione;
5-Amino-7-[4-(1-amino-2-hydroxyethyl)-4,5,6,7-tetrahydrobenzo[b]thiophen-2-yl]-1-cyclopropyl-6-fluoro-8-methyl-1H-quinazoline-2,4-dione;
5-Amino-7-[4-(1-amino-2,2,2-trifluoroethyl)-4,5,6,7-tetrahydrobenzo[b]thiophen-2-yl]-1-cyclopropyl-6-fluoro-8-methyl-1H-quinazoline-2,4-dione;
5-Amino-7-(5-aminomethyl-4,5,6,7-tetrahydrobenzo[b]thiophen-2-yl)-1-cyclopropyl-6-fluoro-8-methyl-1H-quinazoline-2,4-dione;
5-Amino-1-cyclopropyl-6-fluoro-7-(5-hydroxymethyl-4,5,6,7-tetrahydrobenzo[b]thiophen-2-yl)-8-methyl-1H-quinazoline-2,4-dione;
5-Amino-7-[5-(1-amino-2,2,2-trifluoroethyl)-4,5,6,7-tetrahydrobenzo[b]thiophen-2-yl]-1-cyclopropyl-6-fluoro-8-methyl-1H-quinazoline-2,4-dione;
5-Amino-7-(3-aminomethyl-4-trifluoromethylpyrrolidin-1-yl)-1-cyclopropyl-6-fluoro-8-methyl-1H-quinazoline-2,4-dione;
5-Amino-7-[3-(1-amino-2,2,3,3,3-pentafluoropropyl)pyrrolidin-1-yl]-1-cyclopropyl-6-fluoro-8-methyl-1H-quinazoline-2,4-dione;
5-Amino-7-[3-(1-amino-3,3,3-trifluoropropyl)pyrrolidin-1-yl]-1-cyclopropyl-6-fluoro-8-methyl-1H-quinazoline-2,4-dione;
5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazoline-2,4-dione;
5-Amino-7-(3-aminomethyl-4-trifluoromethylpyrrolidin-1-yl)-1-cyclopropyl-6-fluoro-8-methyl-1H-quinazoline-2,4-dione;
5-Amino-7-[3-(1-amino-2,2,3,3,3-pentafluoropropyl)pyrrolidin-1-yl]-1-cyclopropyl-6-fluoro-8-methyl-1H-quinazoline-2,4-dione;
5-Amino-7-[3-(1-amino-3-trifluoropropyl)pyrrolidin-1-yl]-1-cyclopropyl-6-fluoro-8-methyl-1H-quinazoline-2,4-dione;
5-Amino-7-[3-(1-amino-3,3,3-trifluoro-2,2,2-trifluoromethylpropyl)pyrrolidin-1-yl]-1-cyclopropyl-6-fluoro-8-methyl-1H-quinazoline-2,4-dione;
5-Amino-7-[4-(1-aminoethyl)-3,3-difluoropyrrolidin-1-yl-1-cyclopropyl-6-fluoro-8-methyl-1H-quinazoline-2,4-dione;
5-Amino-7-(3-amino-4-ethylpiperidin-1-yl)-1-cyclopropyl-6-fluoro-8-methyl-1H-quinazoline-2,4-dione;
5-Amino-7-[3-(1-amino-2,2,2-trifluoromethoxyethyl)pyrrolidin-1-yl]-1-cyclopropyl-5-difluoromethyl-6-fluoro-8-methyl-1H-quinazoline-2,4-dione;
5-Amino-7-(3-aminomethyl-4-trifluoromethoxypyrrolidin-1-yl)-1-cyclopropyl-6-fluoro-8-methyl-1H-quinazoline-2,4-dione;
5-Amino-7-[3-aminomethyl-4-(2,2,2-trifluoroethyl)pyrrolidin-1-yl]-1-cyclopropyl-6-fluoro-8-methyl-1H-quinazoline-2,4-dione;
5-Amino-7-[3-(1-amino-2-difluoromethyl-3,3-difluoropropyl)pyrrolidin-1-yl]-1-cyclopropyl-6-fluoro-8-methyl-1H-quinazoline-2,4-dione;
5-Amino-7-[3-(1-amino-3-fluoro-2-fluoromethylpropyl)pyrrolidin-1-yl]-1-cyclopropyl-6-fluoro-8-methyl-1H-quinazoline-2,4-dione;
5-Amino-7-[3-(1-amino-3,3-difluoropropyl)pyrrolidin-1-yl]-1-cyclopropyl-6-fluoro-8-methyl-1H-quinazoline-2,4-dione;
5-Amino-1-cyclopropyl-7-(3,3-difluoro-4-hydroxymethylpyrrolidin-1-yl)-6-fluoro-8-methyl-1H-quinazoline-2,4-dione;
5-Amino-1-cyclopropyl-7-[7-(1,2-dihydroxyethyl)-5-azaspiro[2.4]hept-5-yl]-6-fluoro-8-methyl-1H-quinazoline-2,4-dione;
5-Amino-1-cyclopropyl-7-(5,5-difluoro-4-hydroxymethyl-4,5,6,7-tetrahydrobenzo[b]thiophen-2-yl)-6-fluoro-8-methyl-1H-quinazoline-2,4-dione;
5-Amino-7-[3-(1-amino-2,2,2-trifluoro-1-trifluoromethylethyl)pyrrolidin-1-yl]-1-cyclopropyl-6-fluoro-8-methyl-1H-quinazoline-2,4-dione;
5-Amino-7-[3-aminomethyl-4-(3,3,3-trifluoro-2-trifluoromethylpropyl)pyrrolidin-1-yl]-1-cyclopropyl-6-fluoro-8-methyl-1H-quinazoline-2,4-dione;
5-Amino-7-[3-(1-amino-4,4,4-trifluoro-3-trifluoromethyl-but-2-enyl)pyrrolidin-1-yl]-1-cyclopropyl-6-fluoro-8-methyl-1H-quinazoline-2,4-dione;
5-Amino-7-[3-(1-amino-4,4,4-trifluoro-3-trifluoromethylbutyl)pyrrolidin-1-yl]-1-cyclopropyl-6-fluoro-8-methyl-1H-quinazoline-2,4-dione;
5-Amino-7-[3-(1-amino-2-hydroxyethyl)-4-fluoropyrrolidin-1-yl]-1-cyclopropyl-6-fluoro-8-methyl-1H-quinazoline-2,4-dione;
5-Amino-7-[3-(1-amino-2,2,2-trifluoroethyl)-4-fluoropyrrolidin-1-yl]-1-cyclopropyl-6-fluoro-8-methyl-1H-quinazoline-2,4-dione;
5-Amino-7-(3-aminomethyl-4-difluoromethoxypyrrolidin-1-yl)-1-cyclopropyl-6-fluoro-8-methyl-1H-quinazoline-2,4-dione;
5-Amino-7-(4-aminomethyl-5,5-difluoro-4,5,6,7-tetrahydrobenzo[b]thiophen-2-yl)-1-cyclopropyl-6-fluoro-8-methyl-1H-quinazoline-2,4-dione;
5-Amino-7-(4-amino-5,5-difluorooctahydrocyclohepta[c]pyrrol-2-yl)-1-cyclopropyl-6-fluoro-8-methyl-1H-quinazoline-2,4-dione;
5-Amino-1-cyclopropyl-7-(5,5-difluoro-4-hydroxyoctahydrocyclohepta[c]pyrrol-2-yl)-6-fluoro-8-methyl-1H-quinazoline-2,4-dione;
5-Amino-7-[5-(1-aminoethyl)thiophen-3-yl]-1-cyclopropyl-6-fluoro-8-methyl-1H-quinazoline-2,4-dione;
5-Amino-7-(5-aminomethylthiophen-3-yl)-1-cyclopropyl-6-fluoro-8-methyl-1H-quinazoline-2,4-dione;
5-Amino-7-(4-amino-5,5-difluoro-4,5,6,7-tetrahydrobenzo[b]thiophen-2-yl)-1-cyclopropyl-6-fluoro-8-methyl-1H-quinazoline-2,4-dione;
5-Amino-1-cyclopropyl-7-(5,5-difluoro-4-hydroxy-4,5,6,7-tetrahydrobenzo[b]thiophen-2-yl)-6-fluoro-8-methyl-1H-quinazoline-2,4-dione;
5-Amino-7-(4-amino-5,5-difluorooctahydroisoindol-2-yl)-1-cyclopropyl-6-fluoro-8-methoxy-1H-quinazoline-2,4-dione;
5-Amino-7-(4-amino-5,5-difluorohexahydrocyclopenta[c]pyrrol-2-yl)-1-cyclopropyl-6-fluoro-8-methoxy-1H-quinazoline-2,4-dione;
5-Amino-7-(5,5-difluoro-4-hydroxyoctahydroisoindol-2-yl)-1-cyclopropyl-6-fluoro-8-methoxy-1H-quinazoline-2,4-dione;
5-Amino-7-(5,5-difluoro-4-hydroxyhexahydrocyclopenta[c]pyrrol-2-yl)-1-cyclopropyl-6-fluoro-8-methoxy-1H-quinazoline-2,4-dione;
5-Amino-7-[3-(1-amino-2-hydroxyethyl)pyrrolidin-1-yl]-1-cyclopropyl-6-fluoro-8-methoxy-1H-quinazoline-2,4-dione;
5-Amino-7-[3-(1-amino-3,3,3-trifluoro-2-trifluoromethylpropyl)pyrrolidin-1-yl]-1-cyclopropyl-6-fluoro-8-methoxy-1H-quinazoline-2,4-dione;
5-Amino-7-(4-aminomethyl-4,5,6,7-tetrahydrobenzo[b]thiophen-2-yl)-1-cyclopropyl-6-fluoro-8-methoxy-1H-quinazoline-2,4-dione;
5-Amino-1-cyclopropyl-6-fluoro-7-(4-hydroxymethyl-4,5,6,7-tetrahydrobenzo[b]thiophen-2-yl)-8-methoxy-1H-quinazoline-2,4-dione;
5-Amino-7-[4-(1-aminoethyl)-4,5,6,7-tetrahydrobenzo[b]thiophen-2-yl]-1-cyclopropyl-6-fluoro-8-methoxy-1H-quinazoline-2,4-dione;
5-Amino-7-[4-(2-amino-]-hydroxyethyl)-4,5,6,7-tetrahydrobenzo[b]thiophen-2-yl]-1-cyclopropyl-6-fluoro-8-methoxy-1H-quinazoline-2,4-dione;
5-Amino-7-[4-(1-amino-2-hydroxyethyl)-4,5,6,7-tetrahydrobenzo[b]thiophen-2-yl]-1-cyclopropyl-6-fluoro-8-methoxy-1H-quinazoline-2,4-dione;
5-Amino-7-[4-(1-amino-2,2,2-trifluoroethyl)-4,5,6,7-tetrahydrobenzo[b]thiophen-2-yl]-1-cyclopropyl-6-fluoro-8-methoxy-1H-quinazoline-2,4-dione;
5-Amino-7-(5-aminomethyl-4,5,6,7-tetrahydrobenzo[b]thiophen-2-yl)-1-cyclopropyl-6-fluoro-8-methoxy-1H-quinazoline-2,4-dione;
5-Amino-1-cyclopropyl-6-fluoro-7-(5-hydroxymethyl-4,5,6,7-tetrahydrobenzo[b]thiophen-2-yl)-8-methoxy-1H-quinazoline-2,4-dione;
5-Amino-7-[5-(1-amino-2,2,2-trifluoroethyl)-4,5,6,7-tetrahydrobenzo[b]thiophen-2-yl]-1-cyclopropyl-6-fluoro-8-methoxy-1H-quinazoline-2,4-dione;
5-Amiino-7-(3-aminomethyl-4-trifluoromethylpyrrolidin-1-yl)-1-cyclopropyl-6-fluoro-8-methoxy-1H-quinazoline-2,4-dione;
5-Amino-7-[3-(1-amino-2,2,3,3,3-pentafluoropropyl)pyrrolidin-1-yl]-1-cyclopropyl-6-fluoro-8-methoxy-1H-quinazoline-2,4-dione;
5-Amino-7-[3-(1-amino-3,3,3-trifluoropropyl)pyrrolidin-1-yl]-1-cyclopropyl-6-fluoro-8-methoxy-1H-quinazoline-2,4-dione;
5-Amino-7-[3-(1-amino-3,3,3-trifluoro-2-trifluoromethylpropyl)pyrrolidin-1-yl]-1-cyclopropyl-6-fluoro-8-methoxy-1H-quinazoline-2,4-dione;
5-Amino-7-(3-aminomethyl-4-trifluoromethylpyrrolidin-1-yl)-1-cyclopropyl-6-fluoro-8-methoxy-1H-quinazoline-2,4-dione;
5-Amino-7-[3-(1-amino-2,2,3,3,3-pentafluoropropyl)pyrrolidin-1-yl]-1-cyclopropyl-6-fluoro-8-methoxy-1H-quinazoline-2,4-dione;
5-Amino-7-[3-(1-amino-3,3,3-trifluoropropyl)pyrrolidin-1-yl]-1-cyclopropyl-6-fluoro-8-methoxy-1H-quinazoline-2,4-dione;
5-Amino-7-[3-(1-amino-3,3,3-trifluoro-2-trifluoromethylpropyl)pyrrolidin-1-yl]-1-cyclopropyl-6-fluoro-8-methoxy-1H-quinazoline-2,4-dione;
5-Amino-7-[4-(1-aminoethyl)-3,3-difluoropyrrolidin-1-yl]-1-cyclopropyl-6-fluoro-8-methoxy-1H-quinazoline-2,4-dione;
5-Amino-7-(3-amino-4-ethylpiperidin-1-yl)-1-cyclopropyl-6-fluoro-8-methoxy-1H-quinazoline-2,4-dione;
5-Amino-7-[3-(1-amino-2-trifluoromethoxyethyl)pyrrolidin-1-yl]-1-cyclopropyl-5-difluoromethyl-6-fluoro-8-methoxy-1H-quinazoline-2,4-dione;
5-Amino-7-(3-aminomethyl-4-trifluoromethoxypyrrolidin-1-yl)-1-cyclopropyl-6-fluoro-8-methoxy-1H-quinazoline-2,4-dione;
5-Amino-7-[3-aminomethyl-4-(2,2,2-trifluoroethyl)pyrrolidin-1-yl]-1-cyclopropyl-6-fluoro-8-methoxy-1H-quinazoline-2,4-dione;
5-Amino-7-[3-(1-amino-2-difluoromethyl-3,3-difluoropropyl)pyrrolidin-1-yl]-1-cyclopropyl-6-fluoro-8-methoxy-1H-quinazoline-2,4-dione;
5-Amino-7-[3-(-amino-3-fluoro-2-fluoromethylpropyl)pyrrolidin-1-yl]-1-cyclopropyl-6-fluoro-8-methoxy-1H-quinazoline-2,4-dione;
5-Amino-7-[3-(1-amino-3,3-difluoropropyl)pyrrolidin-1-yl]-1-cyclopropyl-6-fluoro-8-methoxy-1H-quinazoline-2,4-dione;
5-Amino-1-cyclopropyl-7-(3,3-difluoro-4-hydroxymethylpyrrolidin-1-yl)-6-fluoro-8-methoxy-1H-quinazoline-2,4-dione;
5-Amino-1-cyclopropyl-7-[7-(1,2-dihydroxyethyl)-5-azaspiro[2.4]hept-5-yl]-6-fluoro-8-methoxy-1H-quinazoline-2,4-dione;
5-Amino-1-cyclopropyl-7-(5,5-difluoro-4-hydroxymethyl-4,5,6,7-tetrahydrobenzo[b]thiophen-2-yl)-6-fluoro-8-methoxy-1H-quinazoline-2,4-dione;
5-Amino-7-[3-(1-amino-2,2,2-trifluoro-1-trifluoromethylethyl)pyrrolidin-1-yl]-1-cyclopropyl-6-fluoro-8-methoxy-1H-quinazoline-2,4-dione;
5-Amino-7-[3-aminomethyl-4-(3,3,3-trifluoro-2-trifluoromethylpropyl)pyrrolidin-1-yl]-1-cyclopropyl-6-fluoro-8-methoxy-1H-quinazoline-2,4-dione;
5-Amino-7-[3-(1-amino-4,4,4-trifluoro-3-trifluoromethyl-but-2-enyl)pyrrolidin-1-yl]-1-cyclopropyl-6-fluoro-8-methoxy-1H-quinazoline-2,4-dione;
5-Amino-7-[3-(1-amino-4,4,4-trifluoro-3-trifluoromethylbutyl)pyrrolidin-1-yl]-1-cyclopropyl-6-fluoro-8-methoxy-1H-quinazoline-2,4-dione;
5-Amino-7-[3-(1-amino-2-hydroxyethyl)-4-fluoropyrrolidin-1-yl]-1-cyclopropyl-6-fluoro-8-methoxy-1H-quinazoline-2,4-dione;
5-Amino-7-[3-(1-amino-2,2,2-trifluoroethyl)-4-fluoropyrrolidin-1-yl]-1-cyclopropyl-6-fluoro-8-methoxy-1H-quinazoline-2,4-dione;
5-Amino-7-(3-aminomethyl-4-difluoromethoxypyrrolidin-1-yl)-1-cyclopropyl-6-fluoro-8-methoxy-1H-quinazoline-2,4-dione;
5-Amino-7-(4-aminomethyl-5,5-difluoro-4,5,6,7-tetrahydrobenzo[b]thiophen-2-yl)-1-cyclopropyl-6-fluoro-8-methoxy-1H-quinazoline-2,4-dione;
5-Amino-7-(4-amino-5,5-difluorooctahydrocyclohepta[c]pyrrol-2-yl)-1-cyclopropyl-6-fluoro-8-methoxy-1H-quinazoline-2,4-dione;
5-Amino-1-cyclopropyl-7-(5,5-difluoro-4-hydroxyoctahydrocyclohepta[c]pyrrol-2-yl)-6-fluoro-8-methoxy-1H-quinazoline-2,4-dione;
5-Amino-7-[5-(1-aminoethyl)thiophen-3-yl]-1-cyclopropyl-6-fluoro-8-methoxy-1H-quinazoline-2,4-dione;
5-Amino-7-(5-aminomethylthiophen-3-yl)-1-cyclopropyl-6-fluoro-8-methoxy-1H-quinazoline-2,4-dione;
5-Amino-7-(4-amino-5,5-difluoro-4,5,6,7-tetrahydrobenzo[b]thiophen-2-yl)-1-cyclopropyl-6-fluoro-8-methoxy-1H-quinazoline-2,4-dione;
5-Amino-1-cyclopropyl-7-(5,5-difluoro-4-hydroxy-4,5,6,7-tetrahydrobenzo[b]thiophen-2-yl)-6-fluoro-8-methoxy-1H-quinazoline-2,4-dione;
7-(4-Amino-5,5-difluorooctahydroisoindol-2-yl)-1-cyclopropyl-6-fluoro-5-hydroxy-8-methyl-1H-quinazoline-2,4-dione;
7-(4-Amino-5,5-difluorohexahydrocyclopenta[c]pyrrol-2-yl)-1-cyclopropyl-6-fluoro-5-hydroxy-8-methyl-1H-quinazoline-2,4-dione;
7-(5,5-Difluoro-4-hydroxyoctahydroisoindol-2-yl)-1-cyclopropyl-6-fluoro-5-hydroxy-8-methyl-1H-quinazoline-2,4-dione;
7-(5,5-Difluoro-4-hydroxyhexahydrocyclopenta[c]pyrrol-2-yl)-1-cyclopropyl-6-fluoro-5-hydroxy-8-methyl-1H-quinazoline-2,4-dione;
7-[3-(1-Amino-2-hydroxyethyl)pyrrolidin-1-yl]-1-cyclopropyl-6-fluoro-5-hydroxy-8-methyl-1H-quinazoline-2,4-dione;
7-[3-(1-Amino-3,3,3-trifluoro-2-trifluoromethylpropyl)pyrrolidin-1-yl]-1-cyclopropyl-6-fluoro-5-hydroxy-8-methyl-1H-quinazoline-2,4-dione;
7-(4-Aminomethyl-4,5,6,7-tetrahydrobenzo[b]thiophen-2-yl)-1-cyclopropyl-6-fluoro-5-hydroxy-8-methyl-1H-quinazoline-2,4-dione;
1-Cyclopropyl-6-fluoro-7-(4-hydroxymethyl-4,5,6,7-tetrahydro-benzo[b]thiophen-2-yl)-5-hydroxy-8-methyl-1H-quinazoline-2,4-dione;
7-[4-(1-Aminoethyl)-4,5,6,7-tetrahydrobenzo[b]thiophen-2-yl]-1-cyclopropyl-6-fluoro-5-hydroxy-8-methyl-1H-quinazoline-2,4-dione;
7-[4-(2-Amino-1-hydroxyethyl)-4,5,6,7-tetrahydrobenzo[b]thiophen-2-yl]-1-cyclopropyl-6-fluoro-5-hydroxy-8-methyl-1H-quinazoline-2,4-dione;
7-[4-(1-Amino-2-hydroxyethyl)-4,5,6,7-tetrahydrobenzo[b]thiophen-2-yl]-1-cyclopropyl-6-fluoro-5-hydroxy-8-methyl-1H-quinazoline-2,4-dione;
7-[4-(1-Amino-2,2,2-trifluoroethyl)-4,5,6,7-tetrahydrobenzo[b]thiophen-2-yl]-1-cyclopropyl-6-fluoro-5-hydroxy-8-methyl-1H-quinazoline-2,4-dione;
7-(5-Aminomethyl-4,5,6,7-tetrahydrobenzo[b]thiophen-2-yl)-1-cyclopropyl-6-fluoro-5-hydroxy-8-methyl-1H-quinazoline-2,4-dione;
1-Cyclopropyl-6-fluoro-7-(5-hydroxymethyl-4,5,6,7-tetrahydro-benzo[b]thiophen-2-yl)-5-hydroxy-8-methyl-1H-quinazoline-2,4-dione;
7-[5-(1-Amino-2,2,2-trifluoroethyl)-4,5,6,7-tetrahydrobenzo[b]thiophen-2-yl]-1-cyclopropyl-6-fluoro-5-hydroxy-8-methyl-1H-quinazoline-2,4-dione;
7-[5-(1-Aminoethyl)-4,5,6,7-tetrahydrobenzo[b]thiophen-2-yl]-1-cyclopropyl-6-fluoro-5-hydroxy-8-methyl-1H-quinazoline-2,4-dione;
7-[5-(1-Amino-2-hydroxyethyl)-4,5,6,7-tetrahydrobenzo[b]thiophen-2-yl]-1-cyclopropyl-6-fluoro-5-hydroxy-8-methyl-1H-quinazoline-2,4-dione;
7-(3-Aminomethyl-4-trifluoromethylpyrrolidin-1-yl)-1-cyclopropyl-6-fluoro-5-hydroxy-8-methyl-1H-quinazoline-2,4-dione;
7-[3-(1-Amino-2,2,3,3,3-pentafluoropropyl)pyrrolidin-1-yl]-1-cyclopropy-6-fluoro-5-hydroxy-8-methyl-1H-quinazoline-2,4-dione;
7-[3-(1-Amino-3,3,3-trifluoropropyl)pyrrolidin-1-yl]-1-cyclopropyl-6-fluoro-5-hydroxy-8-methyl-1H-quinazoline-2,4-dione;
7-[3-(1-Amino-3,3,3-trifluoro-2-trifluoromethylpropyl)pyrrolidin-1-yl]-1-cyclopropyl-6-fluoro-5-hydroxy-8-methyl-1H-quinazoline-2,4-dione;
7-[4-(1-Aminoethyl)-3,3-difluoropyrrolidin-1-yl)-1-cyclopropyl-6-fluoro-5-hydroxy-8-methyl-1H-quinazoline-2,4-dione;
7-(3-Amino-4-ethylpiperidin-1-yl)-1-cyclopropyl-6-fluoro-5-hydroxy-8-methyl-1H-quinazoline-2,4-dione;
7-[3-(1-Amino-2-trifluoromethoxyethyl)pyrrolidin-1-yl]-1-cyclopropyl-6-fluoro-5-hydroxy-8-methyl-1H-quinazoline-2,4-dione;
7-(3-Aminomethyl-4-trifluoromethoxypyrrolidin-1-yl)-1-cyclopropyl-6-fluoro-5-hydroxy-8-methyl-1H-quinazoline-2,4-dione;
7-[3-Aminomethyl-4-(2,2,2-trifluoroethyl)pyrrolidin-1-yl]-1-cyclopropyl-6-fluoro-5-hydroxy-8-methyl-1H-quinazoline-2,4-dione;
7-[3-(1-Amino-2-difluoromethyl-3,3-difluoropropyl)pyrrolidin-1-yl]-1-cyclopropyl-6-fluoro-5-hydroxy-8-methyl-1H-quinazoline-2,4-dione;
7-[3-(1-Amino-3-fluoro-2-fluoromethylpropyl)pyrrolidin-1-yl]-1-cyclopropyl-6-fluoro-5-hydroxy-8-methyl-1H-quinazoline-2,4-dione;
7-[3-(1-Amino-3,3-difluoropropyl)pyrrolidin-1-yl]-1-cyclopropyl-6-fluoro-5-hydroxy-8-methyl-1H-quinazoline-2,4-dione;
1-Cyclopropyl-7-(3,3-difluoro-4-hydroxymethylpyrrolidin-1-yl)-6-fluoro-5-hydroxy-8-methyl-1H -quinazoline-2,4-dione;
1-Cyclopropyl-7-[7-(1,2-dihydroxyethyl)-5-azaspiro[2.4]hept-5-yl]-6-fluoro-5-hydroxy-8-methyl-1H-quinazoline-2,4-dione;
1-Cyclopropyl-7-(5,5-difluoro-4-hydroxymethyl-4,5,6,7-tetrahydrobenzo[b]thiophen-2-yl)-6-fluoro-5-hydroxy-8-methyl-1H-quinazoline-2,4-dione;
7-[3-(1-Amino-2,2,2-trifluoro-1-trifluoromethylethyl)pyrrolidin-1-yl]-1-cyclopropyl-6-fluoro-5-hydroxy-8-methyl-1H-quinazoline-2,4-dione;
7-[3-Aminomethyl-4-(3,3,3-trifluoro-2-trifluoromethylpropyl)pyrrolidin-1-yl]-1-cyclopropyl-6-fluoro-5-hydroxy-8-methyl-1H-quinazoline-2,4-dione;
7-[3-(1-Amino-4,4,4-trifluoro-3-trifluoromethylbut-2-enyl)pyrrolidin-1-yl]-1-cyclopropyl-6-fluoro-5-hydroxy-8-methyl-1H-quinazoline-2,4-dione;
7-[3-(1-Amino-4,4,4-trifluoro-3-trifluoromethylbutyl)pyrrolidin-1-yl]-1-cyclopropyl-6-fluoro-5-hydroxy-8-methyl-1H-quinazoline-2,4-dione;
7-[3-(1-Amino-2-hydroxyethyl)-4-fluoropyrrolidin-1-yl]-1-cyclopropyl-6-fluoro-5-hydroxy-8-methyl-1H-quinazoline-2,4-dione;
7-[3-(1-Amino-2,2,2-trifluoroethyl)-4-fluoropyrrolidin-1-yl]-1-cyclopropyl-6-fluoro-5-hydroxy-8-methyl-1H-quinazoline-2,4-dione;
7-(3-Aminomethyl-4-difluoromethoxypyrrolidin-1-yl)-1-cyclopropyl-6-fluoro-5-hydroxy-8-methyl-1H-quinazoline-2,4-dione;
7-(4-Aminomethyl-5,5-difluoro-4,5,6,7-tetrahydrobenzo[b]thiophen-2-yl)-1-cyclopropyl-6-fluoro-5-hydroxy-8-methyl-1H-quinazoline-2,4-dione;
7-(4-Amino-5,5-difluorooctahydrocyclohepta[c]pyrrol-2-yl)-1-cyclopropyl-6-fluoro-5-hydroxy-8-methyl-1H-quinazoline-2,4-dione;
1-Cyclopropyl-7-(5,5-difluoro-4-hydroxyoctahydrocyclohepta[c]pyrrol-2-yl)-6-fluoro-5-hydroxy-8-methyl-1H-quinazoline-2,4-dione;
7-[5-(1-Aminoethyl)thiophen-3-yl]-1-cyclopropyl-6-fluoro-5-hydroxy-8-methyl-1H-quinazoline-2,4-dione;
7-(5-Aminomethylthiophen-3-yl)-1-cyclopropyl-6-fluoro-5-hydroxy-8-methyl-1H-quinazoline-2,4-dione;
7-(4-Amino-5,5-difluoro-4,5,6,7-tetrahydrobenzo[b]thiophen-2-yl)-1-cyclopropyl-6-fluoro-5-hydroxy-8-methyl-1H-quinazoline-2,4-dione;
1-Cyclopropyl-7-(5,5-difluoro-4-hydroxy-4,5,6,7-tetrahydrobenzo[b]thiophen-2-yl)-6-fluoro-5-hydroxy-8-methyl-1H-quinazoline-2,4-dione;
7-(4-Amino-5,5-difluorooctahydroisoindol-2-yl)-1-cyclopropyl-6-fluoro-5-methoxy-8-methyl-1H-quinazoline-2,4-dione;
7-(4-Amino-5,5-difluorohexahydrocyclopenta[c]pyrrol-2-yl)-1-cyclopropyl-6-fluoro-5-methoxy-8-methyl-1H-quinazoline-2,4-dione;
7-(5,5-Difluoro-4-hydroxyoctahydroisoindol-2-yl)-1-cyclopropyl-6-fluoro-5-methoxy-8-methyl-1H-quinazoline-2,4-dione;
7-(5,5-Difluoro-4-hydroxyhexahydrocyclopenta[c]pyrrol2-yl)-1-cyclopropyl-6-fluoro-5-methoxy-8-methyl-1H-quinazoline-2,4-dione;
7-[3-(1-Amino-2-hydroxyethyl)pyrrolidin-1-yl]-1-cyclopropyl-6-fluoro-5-methoxy-8-methyl-1H-quinazoline-2,4-dione;
7-[3-(1-Amino-3,3,3-trifluoro-2-trifluoromethylpropyl)pyrrolidin-1-yl]-]-cyclopropyl-6-fluoro-5-emthoxy-8-methyl-1H-quinazoline-2,4-dione;
7-(4-Aminomethyl-4,5,6,7-tetrahydrobenzo[b]thiophen-2-yl)-1-cyclopropyl-6-fluoro-5-methoxy-8-methyl-1H-quinazoline-2,4-dione;
1-Cyclopropyl-6-fluoro-7-(4-hydroxymethyl-4,5,6,7-tetrahydrobenzo[b]thiophen-2-yl)-5-methoxy-8-methyl-1H-quinazoline-2,4-dione;
7-[4-(1-Aminoethyl)-4,5,6,7-tetrahydrobenzo[b]thiophen-2-yl]-1-cyclopropyl-6-fluoro-5-methoxy-8-methyl-1H-quinazoline-2,4-dione;
7-[4-(2-Amino-1-hydroxyethyl)-4,5,6,7-tetrahydrobenzo[b]thiophen-2-yl]-1-cyclopropyl-6-fluoro-5-methoxy-8-methyl-1H-quinazoline-2,4-dione;
7-[4-(1-Amino-2-hydroxyethyl)-4,5,6,7-tetrahydrobenzo[b]thiophen-2-yl]-1-cyclopropyl-6-fluoro-5-methoxy-8-methyl-1H-quinazoline-2,4-dione;
7-[4-(1-Amino-2,2,2-trifluoroethyl)-4,5,6,7-tetrahydrobenzo[b]thiophen-2-yl]-1′-cyclopropyl-6-fluoro-5-methoxy-8-methyl-1H-quinazoline-2,4-dione;
7-(5-Aminomethyl-4,5,6,7-tetrahydrobenzo[b]thiophen-2-yl)-1-cyclopropyl-6-fluoro-5-methoxy-8-methyl-1H-quinazoline-2,4-dione;
1-Cyclopropyl-6-fluoro-7-(5-hydroxymethyl-4,5,6,7-tetrahydrobenzo[b]thiophen-2-yl)-5-methoxy-8-methyl-1H-quinazoline-2,4-dione;
7-[5-(-Amino-2,2,2-trifluoroethyl)-4,5,6,7-tetrahydrobenzo[b]thiophen-2-yl]-1-cyclopropyl-6-fluoro-5-methoxy-8-methyl-1H-quinazoline-2,4-dione;
7-[5-(1-Aminoethyl)-4,5,6,7-tetrahydrobenzo[b]thiophen-2-yl]-1-cyclopropyl-6-fluoro-5-methoxy-8-methyl-1H-quinazoline-2,4-dione;
7-[5-(1-Amino-2-hydroxyethyl)-4,5,6,7-tetrahydrobenzo[b]thiophen-2-yl]-1-cyclopropyl-6-fluoro-5-methoxy-8-methyl-1H-quinazoline-2,4-dione;
7-(3-Aminomethyl-4-trifluoromethylpyrrolidin-1-yl)-1-cyclopropyl-6-fluoro-5-methoxy-8-methyl-1H-quinazoline-2,4-dione;
7-[3-(1-Amino-2,2,3,3,3-pentafluoropropyl)pyrrolidin-1-yl]-1-cyclopropy-6-fluoro-5-methoxy-8-methyl-1H-quinazoline-2,4-dione;
7-[3-(1-Amino-3,3,3-trifluoropropyl)pyrrolidin-1-yl]-1-cyclopropyl-6-fluoro-5-methoxy-8-methyl-1H-quinazoline-2,4-dione;
7-[3-(1-Amino-3,3,3-trifluoro-2-trifluoromethylpropyl)pyrrolidin-1-yl]-1-cyclopropyl-6-fluoro-5-methoxy-8-methyl-1H-quinazoline-2,4-dione;
7-[4-(1-Aminoethyl)-3,3-difluoropyrrolidin-1-yl]-1-cyclopropyl-6-fluoro-5-methoxy-8-methyl-1H-quinazoline-2,4-dione;
7-(3-Amino-4-ethylpiperidin-1-yl)-1-cyclopropyl-6-fluoro-5-methoxy-8-methyl-1H-quinazoline-2,4-dione;
7-[3-(1-Amino-2-trifluoromethoxyethyl)pyrrolidin-1-yl]-1-cyclopropyl-6-fluoro-5-methoxy-8-methyl-1H-quinazoline-2,4-dione;
7-(3-Aminomethyl-4-trifluoromethoxypyrrolidin-1-yl)-1-cyclopropyl-6-fluoro-5-methoxy-8-methyl-1H-quinazoline-2,4-dione;
7-[3-Aminomethyl-4-(2,2,2-trifluoroethyl)pyrrolidin-1-yl]-1-cyclopropyl-6-fluoro-5-methoxy-8-methyl-1H-quinazoline-2,4-dione;
7-[3-(1-Amino-2-difluoromethyl-3,3-difluoropropyl)pyrrolidin-1-yl]-1-cyclopropyl-6-fluoro-5-methoxy-8-methyl-1H-quinazoline-2,4-dione;
7-[3-(1-Amino-3-fluoro-2-fluoromethylpropyl)pyrrolidin-1-yl]-1-cyclopropyl-6-fluoro-5-methoxy-8-methyl-1H-quinazoline-2,4-dione;
7-[3-(1-Amino-3,3-difluoropropyl)pyrrolidin-1-yl]-1-cyclopropyl-6-fluoro-5-methoxy-8-methyl-1H-quinazoline-2,4-dione;
1-Cyclopropyl-7-(3,3-difluoro-4-hydroxymethylpyrrolidin-1-yl)-6-fluoro-5-methoxy-8-methyl-1H-quinazoline-2,4-dione;
1-Cyclopropyl-7-[7-(1,2-dihydroxyethyl)-5-azaspiro[2.4]hept-5-yl]-6-fluoro-5-methoxy-8-methyl-1H-quinazoline-2,4-dione;
1-Cyclopropyl-7-(5,5-difluoro-4-hydroxymethyl-4,5,6,7-tetrahydrobenzo[b]thiophen-2-yl)-6-fluoro-5-methoxy-8-methyl-1H-quinazoline-2,4-dione;
7-[3-(1-Amino-2,2,2-trifluoro-1-trifluoromethylethyl)pyrrolidin-1-yl]-1-cyclopropyl-6-fluor6-5-methoxy-8-methyl-1H-quinazoline-2,4-dione;
7-[3-Aminomethyl-4-(3,3,3-trifluoro-2-trifluoromethylpropyl)pyrrolidin-1-yl]-1-cyclopropyl-6-fluoro-5-methoxy-8-methyl-1H-quinazoline-2,4-dione;
7-[3-(1-Amino-4,4,4-trifluoro-3-trifluoromethyl-but-2-enyl)pyrrolidin-1-yl]-1-cyclopropyl-6-fluoro-5-methoxy-8-methyl-1H-quinazoline-2,4-dione;
7-[3-(1-Amino-4,4,4-trifluoro-3-trifluoromethylbutyl)pyrrolidin-1-yl]-1-cyclopropyl-6-fluoro-5-methoxy-8-methyl-1H-quinazoline-2,4-dione;
7-[3-(1-Amino-2-hydroxyethyl)-4-fluoropyrrolidin-1-yl]-1-cyclopropyl-6-fluoro-5-methoxy-8-methyl-1H-quinazoline-2,4-dione;
7-[3-(1-Amino-2,2,2-trifluoroethyl)-4-fluoropyrrolidin-1-yl]-1-cyclopropyl-6-fluoro-5-methoxy-8-methyl-1H-quinazoline-2,4-dione;
7-(3-Aminomethyl-4-difluoromethoxypyrrolidin-1-yl)-1-cyclopropyl-6-fluoro-5-methoxy-8-methyl-1H-quinazoline-2,4-dione;
7-(4-Aminomethyl-5,5-difluoro-4,5,6,7-tetrahydrobenzo[b]thlophen-2-yl)-1-cyclopropyl-6-fluoro-5-methoxy-8-methyl-1H-quinazoline-2,4-dione;
7-(4-Amino-5,5-difluorooctahydrocyclohepta[c]pyrrol-2-yl)-1-cyclopropyl-6-fluoro-5-methoxy-8-methyl-1H-quinazoline-2,4-dione;
1-Cyclopropyl-7-(5,5-difluoro-4-hydroxyoctahydrocyclohepta[c]pyrrol-2-yl)-6-fluoro-5-methoxy-8-methyl-1H-quinazoline-2,4-dione;
7-[5-(1-Aminoethyl)thiophen-3-yl]-1-cyclopropyl-6-fluoro-5-hydroxy-8-methyl-1H-quinazoline-2,4-dione;
7-(5-Aminomethylthiophen-3-yl)-1-cyclopropyl-6-fluoro-5-methoxy-8-methyl-1H-quinazoline-2,4-dione;
7-(4-Amino-5,5-difluoro-4,5,6,7-tetrahydrobenzo[b]thiophen-2-yl)-1-cyclopropyl-6-fluoro-5-methoxy-8-methyl-1H-quinazoline-2,4-dione;
1-Cyclopropyl-7-(5,5-difluoro-4-hydroxy-4,5,6,7-tetrahydrobenzo[b]thiophen-2-yl)-6-fluoro-5-methoxy-8-methyl-1H-quinazoline-2,4-dione;
7-(4-Amino-5,5-difluorooctahydroisoindol-2-yl)-1-cyclopropyl-6-fluoro-8-methyl-1H-quinazoline-2,4-dione;
7-(4-Amino-5,5-difluorohexahydrocyclopenta[c]pyrrol-2-yl)-1-cyclopropyl-6-fluoro-8-methyl-1H-quinazoline-2,4-dione;
7-(5,5-Difluoro-4-hydroxyoctahydroisoindol-2-yl)-1-cyclopropyl-6-fluoro-8-methyl-1H-quinazoline-2,4-dione;
7-(5,5-Difluoro-4-hydroxyhexahydrocyclopenta[c]pyrrol-2-yl)-1-cyclopropyl-6-fluoro-8-methyl-1H-quinazoline-2,4-dione;
7-[3-(1-Amino-2-hydroxyethyl)pyrrolidin-1-yl]-1-cyclopropyl-6-fluoro-8-methyl-1H-quinazoline-2,4-dione;
7-[3-(1-Amino-3,3,3-trifluoro-2-trifluoromethylpropyl)pyrrolidin-1-yl]-1-cyclopropyl-6-fluoro-8-methyl-1H-quinazoline-2,4-dione;
7-(4-Aminomethyl-4,5,6,7-tetrahydrobenzo[b]thiophen-2-yl)-1-cyclopropyl-6-fluoro-8-methyl-1H-quinazoline-2,4-dione;
1-Cyclopropyl-6-fluoro-7-(4-hydroxymethyl-4,5,6,7-tetrahydrobenzo[b]thiophen-2-yl)-8-methyl-1H-quinazoline-2,4-dione;
7-[4-(1-Aminoethyl)-4,5,6,7-tetrahydrobenzo[b]thiophen-2-yl]-1-cyclopropyl-6-fluoro-8-methyl-1H-quinazoline-2,4-dione;
7-[4-(2-Amino-1-hydroxyethyl)-4,5,6,7-tetrahydrobenzo[b]thiophen-2-yl]-251-cyclopropyl-6-fluoro-8-methyl-1H-quinazoline-2,4-dione;
7-[4-(1-Amino-2-hydroxyethyl)-4,5,6,7-tetrahydrobenzo[b]thiophen-2-y]-1-cyclopropyl-6-fluoro-8-methyl-1H-quinazoline-2,4-dione;
7-[4-(1-Amino-2,2,2-trifluoroethyl)-4,5,6,7-tetrahydrobenzo[b]thiophen-2-yl]-1-cyclopropyl-6-fluoro-8-methyl-1H-quinazoline-2,4-dione;
7-(5-Aminomethyl-4,5,6,7-tetrahydrobenzo[b]thiophen-2-yl)-1-cyclopropyl-6-fluoro-8-methyl-1H-quinazoline-2,4-dione;
1-Cyclopropyl-6-fluoro-7-(5-hydroxymethyl-4,5,6,7-tetrahydrobenzo[b]thiophen-2-yl)-8-methyl-1H-quinazoline-2,4-dione;
7-[5-(1-Amino-2,2,2-trifluoroethyl)-4,5,6,7-tetrahydrobenzo[b]thiophen-2-yl]-1-cyclopropyl-6-fluoro-8-methyl-1H-quinazoline-2,4-dione;
7-[5-(1-Aminoethyl)-4,5,6,7-tetrahydrobenzo[b]thiophen-2-yl]-1-cyclopropyl-6-fluoro-8-methyl-1H-quinazoline-2,4-dione;
7-[5-(1-Amino-2-hydroxyethyl)-4,5,6,7-tetrahydrobenzo[b]thiophen-2-yl]-1-cyclopropyl-6-fluoro-8-methyl-1H-quinazoline-2,4-dione;
7-(3-Aminomethyl-4-trifluoromethylpyrrolidin-1-yl)-1-cyclopropyl-6-fluoro-8-methyl-1H-quinazoline-2,4-dione;
7-[3-(1-Amino-2,2,3,3,3-pentafluoropropyl)pyrrolidin-1-yl]-1-cyclopropyl-6-fluoro-8-methyl-1H-quinazoline-2,4-dione;
7-[3-(1-Amino-3,3,3-trifluoropropyl)pyrrolidin-1-yl]-1-cyclopropyl-6-fluoro-8-methyl-1H-quinazoline-2,4-dione;
7-[3-(1-Amino-3,3,3-trifluoro-2-trifluoromethylpropyl)pyrrolidin-1-yl]-1-cyclopropyl-6-fluoro-8-methyl-1H-quinazoline-2,4-dione;
7-[4-(1-Aminoethyl)-3,3-difluoropyrrolidin-1-yl]-1-cyclopropyl-6-fluoro-8-methyl-1H-quinazoline-2,4-dione;
7-(3-Amino-4-ethylpiperidin-1-yl)-1-cyclopropyl-6-fluoro-8-methyl-1H-quinazoline-2,4-dione;
7-[3-(1-Amino-2-trifluoromethoxyethyl)pyrrolidin-1-yl]-1-cyclopropyl-6-fluoro-8-methyl-1H-quinazoline-2,4-dione;
7-(3-Aminomethyl-4-trifluoromethoxypyrrolidin-1-yl)-1-cyclopropyl-6-fluoro-8-methyl-1H-quinazoline-2,4-dione;
7-[3-Aminomethyl-4-(2,2,2-trifluoroethyl)pyrrolidin-1-yl]-1-cyclopropyl-6-fluoro-8-methyl-1H-quinazoline-2,4-dione;
7-[3-(1-Amino-2-difluoromethyl-3,3-difluoropropyl)pyrrolidin-1-yl]-1-cyclopropyl-6-fluoro-8-methyl-1H-quinazoline-2,4-dione;
7-[3-(1-Amino-3-fluoro-2-fluoromethylpropyl)pyrrolidin-1-yl]-1-cyclopropyl-6-fluoro-8-methyl-1H-quinazoline-2,4-dione
7-[3-(1-Amino-3,3-difluoropropyl)pyrrolidin-1-yl]-1-cyclopropyl-6-fluoro-8-methyl-1H-quinazoline-2,4-dione;
1-Cyclopropyl-7-(3,3-difluoro-4-hydroxymethylpyrrolidin-1-yl)-6-fluoro-8-methyl-1H-quinazoline-2,4-dione;
1-Cyclopropyl-7-[7-(1,2-dihydroxyethyl)-5-azaspiro[2.4]hept-5-yl]-6-fluoro-8-methyl-1H-quinazoline-2,4-dione;
1-Cyclopropyl-7-(5,5-difluoro-4-hydroxymethyl-4,5,6,7-tetrahydrobenzo[b]thiophen-2-yl)-6-fluoro-8-methyl-1H-quinazoline-2,4-dione;
7-[3-(1-Amino-2,2,2-trifluoro-1-trifluoromethylethyl)pyrrolidin-1-yl]-1-cyclopropyl-6-fluoro-8-methyl-1H-quinazoline-2,4-dione;
7-[3-Aminomethyl-4-(3,3,3-trifluoro-2-trifluoromethylpropyl)pyrrolidin-1-yl]-1-cyclopropyl-6-fluoro-8-methyl-1H-quinazoline-2,4-dione;
7-[3-(1-Amino-4,4,4-trifluoro-3-trifluoromethyl-but-2-enyl)pyrrolidin-1-yl]-1-cyclopropyl-6-fluoro-8-methyl-1H-quinazoline-2,4-dione;
7-[3-(1-Amino-4,4,4-trifluoro-3-trifluoromethylbutyl)pyrrolidin-1-yl]-1-cyclopropyl-6-fluoro-8-methyl-1H-quinazoline-2,4-dione;
7-[3-(1-Amino-2-hydroxyethyl)-4-fluoropyrrolidin-1-yl]-1-cyclopropyl-6-fluoro-8-methyl-1H-quinazoline-2,4-dione;
7-[3-(1-Amino-2,2,2-trifluoroethyl)-4-fluoropyrrolidin-1-yl]-1-cyclopropyl-6-fluoro-8-methyl-1H-quinazoline-2,4-dione;
7-(3-Aminomethyl-4-difluoromethoxypyrrolidin-1-yl)-1-cyclopropyl-6-fluoro-8-methyl-1H-quinazoline-2,4-dione;
7-(4-Aminomethyl-5,5-difluoro-4,5,6,7-tetrahydrobenzo[b]thiophen-2-yl)-1-cyclopropyl-6-fluoro-8-methyl-1H-quinazoline-2,4-dione;
7-(4-Amino-5,5-difluorooctahydrocyclohepta[c]pyrrol-2-yl)-1-cyclopropyl-6-fluoro-8-methyl-1H-quinazoline-2,4-dione;
1-Cyclopropyl-7-(5,5-difluoro-4-hydroxyoctahydrocyclohepta[c]pyrrol-2-yl)-6-fluoro-8-methyl-1H-quinazoline-2,4-dione;
7-[5-(1-Aminoethyl)thiophen-3-yl]-1-cyclopropyl-6-fluoro-8-methyl-1H-quinazoline-2,4-dione;
7-(5-Aminomethylthiophen-3-yl)-1-cyclopropyl-6-fluoro-8-methyl-1H-quinazoline-2,4-dione;
7-(4-Amino-5,5-difluoro-4,5,6,7-tetrahydrobenzo[b]thiophen-2-yl)-1-cyclopropyl-6-fluoro-8-methyl-1H-quinazoline-2,4-dione
1-Cyclopropyl-7-(5,5-difluoro-4-hydroxy-4,5,6,7-tetrahydrobenzo[b]thiophen-2-yl)-6-fluoro-8-methyl-1H-quinazoline-2,4-dione
7-(4-Amino-5,5-difluorooctahydroisoindol-2-yl)-1-cyclopropyl-6-fluoro-8-methoxy-1H-quinazoline-2,4-dione;
7-(4-Amino-5,5-difluorohexahydrocyclopenta[c]pyrrol-2-yl)-1-cyclopropyl-6-fluoro-8-methoxy-1H-quinazoline-2,4-dione;
7-(5,5-Difluoro-4-hydroxyoctahydroisoindol-2-yl)-1-cyclopropyl-6-fluoro-8-methoxy-1H-quinazoline-2,4-dione;
7-(5,5-Difluoro-4-hydroxyhexahydrocyclopenta[c]pyrrol-2-yl)-1-cyclopropyl-6-fluoro-8-methoxy-1H-quinazoline-2,4-dione;
7-[3-(1-Amino-2-hydroxyethyl)pyrrolidin-1-yl]-1-cyclopropyl-6-fluoro-8-methoxy-1H-quinazoline-2,4-dione;
7-[3-(1-Amino-3,3,3-trifluoro-2-trifluoromethylpropyl)pyrrolidin-1-yl]-1-cyclopropyl-6-fluoro-8-methoxy-1H-quinazoline-2,4-dione;
7-(4-Aminomethyl-4,5,6,7-tetrahydrobenzo[b]thiophen-2-yl)-1-cyclopropyl-6-fluoro-8-methoxy-1H-quinazoline-2,4-dione;
1-Cyclopropyl-6-fluoro-7-(4-hydroxymethyl-4,5,6,7-tetrahydro-benzo[b]thiophen-2-yl)-8-methoxy-1H-quinazoline-2,4-dione;
7-[4-(1-Aminoethyl)-4,5,6,7-tetrahydrobenzo[b]thiophen-2-yl]-1-cyclopropyl-6-fluoro-8-methoxy-1H-quinazoline-2,4-dione;
7-[4-(2-Amino-1-hydroxyethyl)-4,5,6,7-tetrahydrobenzo[b]thiophen-2-yl]-1-cyclopropyl-6-fluoro-8-methoxy-1H-quinazoline-2,4-dione;
7-[4-(1-Amino-2-hydroxyethyl)-4,5,6,7-tetrahydrobenzo[b]thiophen-2-yl]-1-cyclopropyl-6-fluoro-8-methoxy-1H-quinazoline-2,4-dione;
7-[4-(1-Amino-2,2,2-trifluoroethyl)-4,5,6,7-tetrahydrobenzo[b]thiophen-2-yl]-1-cyclopropyl-6-fluoro-8-methoxy-1H-quinazoline-2,4-dione;
7-(5-Aminomethyl-4,5,6,7-tetrahydrobenzo[b]thiophen-2-yl)-1-cyclopropyl-6-fluoro-8-methoxy-1H-quinazoline-2,4-dione;
1-Cyclopropyl-6-fluoro-7-(5-hydroxymethyl-4,5,6,7-tetrahydrobenzo[b]thiophen-2-yl)-8-methoxy-1H-quinazoine-2,4-dione;
7-[5-(1-Amino-2,2,2-trifluoroethyl)-4,5,6,7-tetrahydrobenzo[b]thiophen-2-yl]-1-cyclopropyl-6-fluoro-8-methoxy-1H-quinazoline-2,4-dione;
7-[5-(1-Aminoethyl)-4,5,6,7-tetrahydrobenzo[b]thiophen-2-yl]-1-cyclopropyl-6-fluoro-8-methoxy-1H-quinazoline-2,4-dione;
7-[5-(1-Amino-2-hydroxyethyl)-4,5,6,7-tetrahydrobenzo[b]thiophen-2-yl]-1-cyclopropyl-6-fluoro-8-methoxy-1H-quinazoline-2,4-dione;
7-(3-Aminomethyl-4-trifluoromethylpyrrolidin-1-yl)-1-cyclopropyl-6-fluoro-8-methoxy-1H-quinazoline-2,4-dione;
7-[3-(1-Amino-2,2,3,3,3-pentafluoropropyl)pyrrolidin-1-yl]-1-cyclopropyl-6-fluoro-8-methoxy-1H-quinazoline-2,4-dione;
7-[3-(1-Amino-3,3,3-trifluoropropyl)pyrrolidin-1-yl]-1-cyclopropyl-6-fluoro-8-methoxy-1H-quinazoline-2,4-dione;
7-[3-(1-Amino-3,3,3-trifluoro-2-trifluoromethylpropyl)pyrrolidin-1-yl]-1-cyclopropyl-6-fluoro-8-methoxy-1H-quinazoline-2,4-dione;
7-[4-(1-Aminoethyl)-3,3-difluoropyrrolidin-1-yl]-1-cyclopropyl-6-fluoro-8-methoxy-1H-quinazoline-2,4-dione;
7-(3-Amino-4-ethylpiperidin-1-yl)-1-cyclopropyl-6-fluoro-8-methoxy-1H-quinazoline-2,4-dione;
7-[3-(1-Amino-2-trifluoromethoxyethyl)pyrrolidin-1-yl]-1-cyclopropyl-6-fluoro-8-methoxy-1H-quinazoline-2,4-dione;
7-(3-Aminomethyl-4-trifluoromethoxypyrrolidin-1-yl)-1-cyclopropyl-6-fluoro-8-methoxy-1H-quinazoline-2,4-dione;
7-[3-Aminomethyl-4-(2,2,2-trifluoroethyl)pyrrolidin-1-yl]-1-cyclopropyl-6-fluoro-8-methoxy-1H-quinazoline-2,4-dione;
7-[3-(1-Amino-2-difluoromethyl-3,3-difluoropropyl)pyrrolidin-1-yl]-1-cyclopropyl-6-fluoro-8-methoxy-1H-quinazoline-2,4-dione;
7-[3-(1-Amino-3-fluoro-2-fluoromethylpropyl)pyrrolidin-1-yl]-1-cyclopropyl-6-fluoro-8-methoxy-1H-quinazoline-2,4-dione;
7-[3-(1-Amino-3,3-difluoropropyl)pyrrolidin-1-yl]-1-cyclopropyl-6-fluoro-8-methoxy-1H-quinazoline-2,4-dione;
1-Cyclopropyl-7-(3,3-difluoro-4-hydroxymethylpyrrolidin-1-yl)-6-fluoro-8-methoxy-1H-quinazoline-2,4-dione;
1-Cyclopropyl-7-[7-(1,2-dihydroxyethyl)-5-azaspiro[2.4]hept-5-yl]-6-fluoro-8-methoxy-1H-quinazoline-2,4-dione;
1-Cyclopropyl-7-(5,5-difluoro-4-hydroxymethyl-4,5,6,7-tetrahydrobenzo[b]thiophen-2-yl)-6-fluoro-8-methoxy-1H-quinazoline-2,4-dione;
7-[3-(1-Amino-2,2,2-trifluoro-1-trifluoromethylethyl)pyrrolidin-1-yl]-1-cyclopropyl-6-fluoro-8-methoxy-1H-quinazoline-2,4-dione;
7-[3-Aminomethyl-4-(3,3,3-trifluoro-2-trifluoromethylpropyl)pyrrolidin-1-yl]-1-cyclopropyl-6-fluoro-8-methoxy-1H-quinazoline-2,4-dione;
7-[3-(1-Amino-4,4,4-trifluoro-3-trifluoromethylbut-2-enyl)pyrrolidin-1-yl]-1-cyclopropyl-6-fluoro-8-methoxy-1H-quinazoline-2,4-dione;
7-[3-(1-Amino-4,4,4-trifluoro-3-trifluoromethylbutyl)pyrrolidin-1-yl]-1-cyclopropyl-6-fluoro-8-methoxy-1H-quinazoline-2,4-dione;
7-[3-(1-Amino-2-hydroxyethyl)-4-fluoropyrrolidin-1-yl]-1-cyclopropyl-6-fluoro-8-methoxy-1H-quinazoline-2,4-dione;
7-[3-(1-Amino-2,2,2-trifluoroethyl)-4-fluoropyrrolidin-1-yl]-1-cyclopropyl-6-fluoro-8-methoxy-1H-quinazoline-2,4-dione;
7-(3-Aminomethyl-4-difluoromethoxypyrrolidin-1-yl)-1-cyclopropyl-6-fluoro-8-methoxy-1H-quinazoline-2,4-dione;
7-(4-Aminomethyl-5,5-difluoro-4,5,6,7-tetrahydrobenzo[b]thiophen-2-yl)-1-cyclopropyl-6-fluoro-8-methoxy-1H-quinazoline-2,4-dione;
7-(4-Amino-5,5-difluorooctahydrocyclohepta[c]pyrrol-2-yl)-1-cyclopropyl-6-fluoro-8-methoxy-1H-quinazoline-2,4-dione;
1-Cyclopropyl-7-(5,5-difluoro-4-hydroxyoctahydrocyclohepta[c]pyrrol-2-yl)-6-fluoro-8-methyl-1H-quinazoline-2,4-dione;
7-[5-(1-Aminoethyl)thiophen-3-yl]-1-cyclopropyl-6-fluoro-8-methoxy-1H-quinazoline-2,4-dione;
7-(5-Aminomethylthiophen-3-yl)-1-cyclopropyl-6-fluoro-8-methoxy-1H-quinazoline-2,4-dione;
7-(4-Amino-5,5-difluoro-4,5,6,7-tetrahydrobenzo[b]thiophen-2-yl)-1-cyclopropyl-6-fluoro-8-methoxy-1H-quinazoline-2,4-dione;
1-Cyclopropyl-7-(5,5-difluoro-4-hydroxy-4,5,6,7-tetrahydrobenzo[b]thiophen-2-yl)-6-fluoro-8-methoxy-1H-quinazoline-2,4-dione;
1-Cyclopropyl-7-[3-(1,2-dihydroxy-2-methylpropyl)pyrrolidin-1-yl]-6-fluoro-5,8-dimethyl-1H-quinazoline-2,4-dione;
1-Cyclopropyl-6-fluoro-5,8-dimethyl-7-[3-(3,3,3-trifluoro-1,2-dihydroxy-2-trifluoromethylpropyl)pyrrolidin-1-yl]-1H-quinazoline-2,4-dione;
1-Cyclopropyl-6-fluoro-7-{3-[hydroxy(1-hydroxycyclopropyl)methyl]pyrrolidin-1-yl}-5,8-dimethyl-1H-quinazoline-2,4-dione;
1-Cyclopropyl-6-fluoro-7-{3-[hydroxy(1-hydroxycyclopentyl)methyl]pyrrolidin-1-yl}-5,8-dimethyl-1H-quinazoline-2,4-dione;
7-[3-(1-Amino-2-hydroxy-2-methylpropyl)pyrrolidin-1-yl]-1-cyclopropyl-6-fluoro-5,8-dimethyl-1H-quinazoline-2,4-dione;
7-[3-(1-Amino-3,3,3-trifluoro-2-hydroxy-2-trifluoromethylpropyl)pyrrolidin-1-yl]-1-cyclopropyl-6-fluoro-5,8-dimethyl-1H-quinazoline-2,4-dione;
7-{3-[Amino-(1-hydroxycyclopropyl)methyl]pyrrolidin-1-yl}-1-cyclopropyl-6-fluoro-5,8-dimethyl-1H-quinazoline-2,4-dione;
7-{3-[Amino-(1-hydroxycyclopentyl)methyl]pyrrolidin-1-yl}-1-cyclopropyl-6-fluoro-5,8-dimethyl-1H-quinazoline-2,4-dione;
1-Cyclopropyl-7-(4,5-dihydroxyhexahydrocyclopenta[c]pyrrol-2-yl)-6-fluoro-5,8-dimethyl-1H-quinazoline-2,4-dione;
1-Cyclopropyl-7-(4,5-dihydroxyoctahydroisoindol-2-yl)-6-fluoro-5,8-dimethyl-1H-quinazoline-2,4-dione;
1-Cyclopropyl-7-(4,5-dihydroxyoctahydrocyclohepta[c]pyrrol-2-yl)-6-fluoro-5,8-dimethyl-1H-quinazoline-2,4-dione;
1-Cyclopropyl-7-(4,5-dihydroxydecahydrocycloocta[c]pyrrol-2-yl)-6-fluoro-5,8-dimethyl-1H-quinazoline-2,4-dione;
5-Amino-1-cyclopropyl-7-[3-(1,2-dihydroxy-2-methylpropyl)pyrrolidin-1-yl]-6-fluoro-8-methyl-1H-quinazoline-2,4-dione;
5-Amino-1-cyclopropyl-6-fluoro-8-methyl-7-[3-(3,3,3-trifluoro-1,2-dihydroxy-2-trifluoromethylpropyl)pyrrolidin-1-yl]-1H-quinazoline-2,4-dione;
5-Amino-1-cyclopropyl-6-fluoro-7-{3-[hydroxy-(1-hydroxycyclopropyl)methyl]pyrrolidin-1-yl}-8-methyl-1H-quinazoline-2,4-dione;
5-Amino-1-cyclopropyl-6-fluoro-7-{3-[hydroxy-(1-hydroxycyclopentyl)methyl]pyrrolidin-1-yl}-8-methyl-1H-quinazoline-2,4-dione;
5-Amino-7-[3-(1-amino-2-hydroxy-2-methylpropyl)pyrrolidin-1-yl]-1-cyclopropyl-6-fluoro-8-methyl-1H-quinazoline-2,4-dione;
5-Amino-7-[3-(1-amino-3,3,3-trifluoro-2-hydroxy-2-trifluoromethylpropyl)pyrrolidin-1-yl]-1-cyclopropyl-6-fluoro-8-methyl-1H-quinazoline-2,4-dione;
5-Amino-7-{3-[amino-(1-hydroxycyclopropyl)methyl]pyrrolidin-1-yl}-1-cyclopropyl-6-fluoro-8-methyl-1H-quinazoline-2,4-dione;
5-Amino-7-{3-[amino-(1-hydroxycyclopentyl)methyl]pyrrolidin-1-yl}-1-cyclopropyl-6-fluoro-8-methyl-1H-quinazoline-2,4-dione;
5-Amino-1-cyclopropyl-7-(4,5-dihydroxyhexahydrocyclopenta[c]pyrrol-2-yl)-6-fluoro-8-methyl-1H-quinazoline-2,4-dione;
5-Amino-1-cyclopropyl-7-(4,5-dihydroxyoctahydroisoindol-2-yl)-6-fluoro-8-methyl-1H-quinazoline-2,4-dione;
5-Amino-1-cyclopropyl-7-(4,5-dihydroxyoctahydrocyclohepta[c]pyrrol-2-yl)-6-fluoro-8-methyl-1H-quinazoline-2,4-dione;
5-Amino-1-cyclopropyl-7-(4,5-dihydroxydecahydrocycloocta[c]pyrrol-2-yl)-6-fluoro-8-methyl-1H-quinazoline-2,4-dione;
1-Cyclopropyl-5-difluoromethyl-7-[3-(1,2-dihydroxy-2-methylpropyl)pyrrolidin-1-yl]-6-fluoro-8-methyl-1H-quinazoline-2,4-dione;
1-Cyclopropyl-5-difluoromethyl-6-fluoro-8-methyl-7-[3-(3,3,3-trifluoro-1,2-dihydroxy-2-trifluoromethylpropyl)pyrrolidin-1-yl]-1H-quinazoline-2,4-dione;
1-Cyclopropyl-5-difluoromethyl-6-fluoro-7-{3-[hydroxy-(1-hydroxycyclopropyl)methyl]pyrrolidin-1-yl}-8-methyl-1H-quinazoline-2,4-dione;
1-Cyclopropyl-5-difluoromethyl-6-fluoro-7-{3-[hydroxy-(1-hydroxycyclopentyl)methyl]pyrrolidin-1-yl}-8-methyl-1H-quinazoline-2,4-dione;
7-[3-(1-Amino-2-hydroxy-2-methylpropyl)pyrrolidin-1-yl]-1-cyclopropyl-5-difluoromethyl-6-fluoro-8-methyl-1H-quinazoline-2,4-dione;
7-[3-(1-Amino-3,3,3-trifluoro-2-hydroxy-2-trifluoromethylpropyl)pyrrolidin-1-yl]-1-cyclopropyl-5-difluoromethyl-6-fluoro-8-methyl-1H-quinazoline-2,4-dione;
7-{3-[Amino-(1-hydroxycyclopropyl)methyl]pyrrolidin-1-yl}-1-cyclopropyl-5-difluoromethyl-6-fluoro-8-methyl-1H-quinazoline-2,4-dione;
7-{3-[Amino-(1-hydroxycyclopentyl)methyl]pyrrolidin-1-yl}-1-cyclopropyl-5-difluoromethyl-6-fluoro-8-methyl-1H-quinazoline-2,4-dione;
1-Cyclopropyl-5-difluoromethyl-7-(4,5-dihydroxyhexahydrocyclopenta[c]pyrrol-2-yl)-6-fluoro-8-methyl-1H-quinazoline-2,4-dione;
1-Cyclopropyl-5-difluoromethyl-7-(4,5-dihydroxyoctahydroisoindol-2-yl)-6-fluoro-8-methyl-1H-quinazoline-2,4-dione;
1-Cyclopropyl-5-difluoromethyl-7-(4,5-dihydroxyoctahydrocyclohepta[c]pyrrol-2-yl)-6-fluoro-8-methyl-1H-quinazoline-2,4-dione;
1-Cyclopropyl-5-difluoromethyl-7-(4,5-dihydroxydecahydrocycloocta[c]pyrrol-2-yl)-6-fluoro-8-methyl-1H-quinazoline-2,4-dione;
1-Cyclopropyl-6-fluoro-7-(3-fluoro-4-hydroxymethylpyrrolidin-1-yl)-5,8-dimethyl-1H-quinazoline-2,4-dione;
1-Cyclopropyl-7-(3,4-dihydroxypiperidin-1-yl)-6-fluoro-5,8-dimethyl-1H-quinazoline-2,4-dione;
1-Cyclopropyl-6-fluoro-7-(3-hydroxymethyl-4-methylpiperidin-1-yl)-5,8-dimethyl-1H-quinazoline-2,4-dione;
1-Cyclopropyl-7-[3-(1,2-dihydroxy-2-methylpropyl)-4-fluoropyrrolidin-1-yl]-6-fluoro-5,8-di methyl-1H-quinazoline-2,4-dione;
1-Cyclopropyl-6-fluoro-7-{3-fluoro-4-[hydroxy-(1-hydroxycyclopropyl)methyl]pyrrolidin-1-yl}-5,8-dimethyl-1H-quinazoline-2,4-dione;
1-Cyclopropyl-6-fluoro-7-{3-fluoro-4-[hydroxy-(1-hydroxycyclopentyl)methyl]pyrrolidin-1-yl}-5,8-dimethyl-1H-quinazoline-2,4-dione;
1-Cyclopropyl-6-fluoro-7-[3-fluoro-4-(3,3,3-trifluoro-1,2-dihydroxy-2-trifluoromethylpropyl)pyrrolidin-1-yl]-5,8-dimethyl-1H-quinazoline-2,4-dione;
1-Cyclopropyl-7-(4-ethyl-3-hydroxypiperidin-1-yl)-6-fluoro-5,8-dimethyl-1H-quinazoline-2,4-dione;
1-Cyclopropyl-7-(4-ethyl-3-hydroxymethylpiperidin-1-yl)-6-fluoro-5,8-dimethyl-1H-quinazoline-2,4-dione;
1-Cyclopropyl-6-fluoro-7-(3-hydroxy-4-methylpiperidin-1-yl)-5,8-dimethyl-1H-quinazoline-2,4-dione;
1-Cyclopropyl-6-fluoro-5,8-dimethyl-7-[3-(2,2,2-trifluoro-1-hydroxyethyl)pyrrolidin-1-yl]-1H-quinazoline-2,4-dione;
1-Cyclopropyl-7-[3-(1,2-dihydroxyethyl)-4-fluoropyrrolidin-1-yl]-6-fluoro-5,8-dimethyl-1H-quinazoline-2,4-dione;
1-Cyclopropyl-6-fluoro-7-(4-hydroxy-3-hydroxymethylpiperidin-1-yl)-5,8-dimethyl-1H-quinazoline-2,4-dione;
5-Amino-1-cyclopropyl-6-fluoro-7-(3-fluoro-4-hydroxymethylpyrrolidin-1-yl)-8-methyl-1H-quinazoline-2,4-dione;
5-Amino-1-cyclopropyl-7-[3-(1,2-dihydroxyethyl)-4-fluoropyrrolidin-1-yl]-6-fluoro-8-methyl-1H-quinazoline-2,4-dione;
5-Amino-1-cyclopropyl-6-fluoro-8-methyl-7-[3-(2,2,2-trifluoro-1-hydroxyethyl)pyrrolidin-1-yl]-1H-quinazoline-2,4-dione;
5-Amino-1-cyclopropyl-7-(3,4-dihydroxypiperidin-1-yl)-6-fluoro-8-methyl-1H-quinazoline-2,4-dione;
1-Cyclopropyl-6-fluoro-7-(4-hydroxy-3-hydroxymethylpiperidin-1-yl)-5,8-dimethyl-1H-quinazoline-2,4-dione;
5-Amino-1-cyclopropyl-6-fluoro-7-(3-hydroxymethyl-4-methylpiperidin-1-yl)-8-methyl-1H-quinazoline-2,4-dione;
5-Amino-1-cyclopropyl-7-[3-(1,2-dihydroxy-2-methylpropyl)-4-fluoropyrrolidin-1-yl]-6-fluoro-8-methyl-1H-quinazoline-2,4-dione;
5-Amino-1-cyclopropyl-6-fluoro-7-{3-fluoro-4-[hydroxy-(1-hydroxycyclopropyl)methyl]pyrrolidin-1-yl}-8-methyl-1H-quinazoline-2,4-dione;
5-Amino-1-cyclopropyl-6-fluoro-7-(3-hydroxy-4-methylpiperidin-1-yl)-8-methyl-1H-quinazoline-2,4-dione;
5-Amino-1-cyclopropyl-7-(4-ethyl-3-hydroxypiperidin-1-yl)-6-fluoro-8-methyl-1H-quinazoline-2,4-dione;
5-Amino-1-cyclopropyl-7-(4-ethyl-3-hydroxymethylpiperidin-1-yl)-6-fluoro-8-methyl-1H-quinazoline-2,4-dione;
5-Amino-1-cyclopropyl-6-fluoro-7-[3-fluoro-4-(3,3,3-trifluoro-1,2-dihydroxy-2-trifluoromethylpropyl)pyrrolidin-1-yl]-8-methyl-1H-quinazoline-2,4-dione;
5-Amino-1-cyclopropyl-6-fluoro-7-{3-fluoro-4-[hydroxy-(1-hydroxycyclopentyl)methyl]pyrrolidin-1-yl}-8-methyl-1H-quinazoline-2,4-dione;
1-Cyclopropyl-5-difluoromethyl-6-fluoro-7-{3-fluoro-4-[hydroxy-(1-hydroxycyclopropyl)methyl]pyrrolidin-1-yl}-8-methyl-1H-quinazoline-2,4-dione;
1-Cyclopropyl-5-difluoromethyl-7-[3-(1,2-dihydroxy-2-methylpropyl)-4-fluoropyrrolidin-1-yl]-6-fluoro-8-methyl-1H-quinazoline-2,4-dione;
1-Cyclopropyl-5-difluoromethyl-6-fluoro-7-(3-hydroxymethyl-4-methylpiperidin-1-yl)-8-methyl-1H-quinazoline-2,4-dione;
1-Cyclopropyl-5-difluoromethyl-7-(3,4-dihydroxypiperidin-1-yl)-6-fluoro-8-methyl-1H-quinazoline-2,4-dione;
1-Cyclopropyl-5-difluoromethyl-6-fluoro-7-(3-fluoro-4-hydroxymethylpyrrolidin-1-yl)-8-methyl-1H-quinazoline-2,4-dione;
1-Cyclopropyl-5-difluoromethyl-6-fluoro-7-{3-fluoro-4-[hydroxy-(1-hydroxycyclopentyl)methyl]pyrrolidin-1-yl}-8-methyl-1H-quinazoline-2,4-dione;
1-Cyclopropyl-5-difluoromethyl-6-fluoro-7-[3-fluoro-4-(3,3,3-trifluoro-1,2-dihydroxy-2-trifluoromethylpropyl) pyrrolidin-1-yl]-8-methyl-1H-quinazoline-2,4-dione;
1-Cyclopropyl-5-difluoromethyl-7-(4-ethyl-3-hydroxymethylpiperidin-1-yl)-6-fluoro-8-methyl-1H-quinazoline-2,4-dione;
1-Cyclopropyl-5-difluoromethyl-7-(4-ethyl-3-hydroxypiperidin-1-yl)-6-fluoro-8-methyl-1H-quinazoline-2,4-dione;
1-Cyclopropyl-5-difluoromethyl-6-fluoro-7-(4-hydroxy-3-hydroxymethylpiperidin-1-yl)-8-methyl-1H-quinazoline-2,4-dione;
1-Cyclopropyl-5-difluoromethyl-6-fluoro-7-(3-hydroxy-4-methylpiperidin-1-yl)-8-methyl-1H-quinazoline-2,4-dione;
1-Cyclopropyl-5-difluoromethyl-7-[3-(1,2-dihydroxyethyl)-4-fluoropyrrolidin-1-yl]-6-fluoro-8-methyl-1H-quinazoline-2,4-dione;
1-Cyclopropyl-5-difluoromethyl-6-fluoro-8-methyl-7-[3-(2,2,2-trifluoro-1-hydroxyethyl)pyrrolidin-1-yl]-1H-quinazoline-2,4-dione;
1-Cyclopropyl-7-[3-(1,2-dihydroxy-2-methylpropyl)-pyrrolidin-1-yl]-6-fluoro-8-methoxy-5-methyl-1H-quinazoline-2,4-dione;
1-Cyclopropyl-6-fluoro-8-methoxy-5-methyl-7-[3-(3,3,3-trifluoro-1,2-dihydroxy-2-trifluoromethylpropyl)pyrrolidin-1-yl]-1H-quinazoline-2,4-dione;
1-Cyclopropyl-6-fluoro-7-{3-[hydroxy-(1-hydroxycyclopropyl)methyl]-pyrrolidin-1-yl}-8-methoxy-5-methyl-1H-quinazoline-2,4-dione;
1-Cyclopropyl-6-fluoro-7-{3-[hydroxy-(1-hydroxycyclopentyl)-methyl]pyrrolidin-1-yl}-8-methoxy-5-methyl-1H-quinazoline-2,4-dione;
7-[3-(1-Amino-2-hydroxy-2-methylpropyl)-pyrrolidin-1-yl]-1-cyclopropyl-6-fluoro-8-methoxy-5-methyl-1H-quinazoline-2,4-dione;
7-[3-(1-Amino-3,3,3-trifluoro-2-hydroxy-2-trifluoromethylpropyl)pyrrolidin-1-yl]-1-cyclopropyl-6-fluoro-8-methoxy-5-methyl-1H-quinazoline-2,4-dione;
7-{3-[Amino-(1-hydroxycyclopropyl)methyl]pyrrolidin-1-yl}-1-cyclopropyl-6-fluoro-8-methoxy-5-methyl-1H-quinazoline-2,4-dione;
7-{3-[Amino-(1-hydroxycyclopentyl)methyl]pyrrolidin-1-yl}-1-cyclopropyl-6-fluoro-8-methoxy-5-methyl-1H-quinazoline-2,4-dione;
1-Cyclopropyl-7-(4,5-dihydroxyhexahydrocyclopenta[c]pyrrol-2-yl)-6-fluoro-8-methoxy-5-methyl-1H-quinazoline-2,4-dione;
1-Cyclopropyl-7-(4,5-dihydroxyoctahydroisoindol-2-yl)-6-fluoro-8-methoxy-5-methyl-1H-quinazoline-2,4-dione;
1-Cyclopropyl-7-(4,5-dihydroxyoctahydrocyclohepta[c]pyrrol-2-yl)-6-fluoro-8-methoxy-5-methyl-1H-quinazoline-2,4-dione;
1-Cyclopropyl-7-(4,5-dihydroxydecahydrocycloocta[c]pyrrol-2-yl)-6-fluoro-8-methoxy-5-methyl-1H-quinazoline-2,4-dione;
5-Amino-1-cyclopropyl-7-[3-(1,2-dihydroxy-2-methylpropyl)pyrrolidin-1-yl]-6-fluoro-8-methoxy-1H-quinazoline-2,4-dione;
5-Amino-1-cyclopropyl-6-fluoro-8-methoxy-7-[3-(3,3,3-trifluoro-1,2-dihydroxy-2-trifluoromethylpropyl)pyrrolidin-1-yl]-1H-quinazoline-2,4-dione;
5-Amino-1-cyclopropyl-6-fluoro-7-{3-[hydroxy-(1-hydroxycyclopropyl)methyl]-pyrrolidin-1-yl}-8-methoxy-1H-quinazoline-2,4-dione;
5-Amino-1-cyclopropyl-6-fluoro-7-{3-[hydroxy-(1-hydroxycyclopentyl)methyl]-pyrrolidin-1-yl}-8-methoxy-1H-quinazoline-2,4-dione;
5-Amino-7-[3-(1-amino-2-hydroxy-2-methylpropyl)pyrrolidin-1-yl]-1-cyclopropyl-6-fluoro-8-methoxy-1H-quinazoline-2,4-dione;
5-Amino-7-[3-(1-amino-3,3,3-trifluoro-2-hydroxy-2-trifluoromethylpropyl)pyrrolidin-1-yl]-1-cyclopropyl-6-fluoro-8-methoxy-1H-quinazoline-2,4-dione;
5-Amino-7-{3-[amino-(1-hydroxycyclopropyl)methyl]pyrrolidin-1-yl}-1-cyclopropyl-6-fluoro-8-methoxy-1H-quinazoline-2,4-dione;
5-Amino-7-{3-[amino-(1-hydroxycyclopentyl)methyl]pyrrolidin-1-yl}-1-cyclopropyl-6-fluoro-8-methoxy-1H-quinazoline-2,4-dione;
5-Amino-1-cyclopropyl-7-(4,5-dihydroxyhexahydrocyclopenta[c]pyrrol-2-yl)-6-fluoro-8-methoxy-1H-quinazoline-2,4-dione;
5-Amino-1-cyclopropyl-7-(4,5-dihydroxyoctahydroisoindol-2-yl)-6-fluoro-8-methoxy-1H-quinazoline-2,4-dione;
5-Amino-1-cyclopropyl-7-(4,5-dihydroxyoctahydrocyclohepta[c]pyrrol-2-yl)-6-fluoro-8-methoxy-1H-quinazoline-2,4-dione;
5-Amino-1-cyclopropyl-7-(4,5-dihydroxydecahydrocycloocta[c]pyrrol-2-yl)-6-fluoro-8-methoxy-1H-quinazoline-2,4-dione;
1-Cyclopropyl-5-difluoromethyl-7-[3-(1,2-dihydroxy-2-methylpropyl)pyrrolidin-1-yl]-6-fluoro-8-methoxy-1H-quinazoline-2,4-dione;
1-Cyclopropyl-5-difluoromethyl-6-fluoro-8-methoxy-7-[3-(3,3,3-trifluoro-1,2-dihydroxy-2-trifluoromethylpropyl)pyrrolidin-1-yl]-1H-quinazoline-2,4-dione;
1-Cyclopropyl-5-difluoromethyl-6-fluoro-7-{3-[hydroxy-(1-hydroxy-cyclopropyl)methyl]pyrrolidin-1-yl}-8-methoxy-1H-quinazoline-2,4-dione;
1-Cyclopropyl-5-difluoromethyl-6-fluoro-7-{3-[hydroxy-(1-hydroxycyclopentyl)methyl]pyrrolidin-1-yl}-8-methoxy-1H-quinazoline-2,4-dione;
7-[3-(1-Amino-2-hydroxy-2-methylpropyl)pyrrolidin-1-yl]-1-cyclopropyl-5-difluoromethyl-6-fluoro-8-methoxy-1H-quinazoline-2,4-dione;
7-[3-(1-Amino-3,3,3-trifluoro-2-hydroxy-2-trifluoromethylpropyl)pyrrolidin-1-yl]-1-cyclopropyl-5-difluoromethyl-6-fluoro-8-methoxy-1H-quinazoline-2,4-dione;
7-{3-[Amino-(1-hydroxycyclopropyl)methyl]pyrrolidin-1-yl}-1-cyclopropyl-5-difluoromethyl-6-fluoro-8-methoxy-1H-quinazoline-2,4-dione;
7-{3-[Amino-(1-hydroxycyclopentyl)methyl]pyrrolidin-1-yl}-1-cyclopropyl-5-difluoromethyl-6-fluoro-8-methoxy-1H-quinazoline-2,4-dione;
1-Cyclopropyl-5-difluoromethyl-7-(4,5-dihydroxyhexahydrocyclopenta[c]pyrrol-2-yl)-6-fluoro-8-methoxy-1H-quinazoline-2,4-dione;
1-Cyclopropyl-5-difluoromethyl-7-(4,5-dihydroxyoctahydroisoindol-2-yl)-6-fluoro-8-methoxy-1H-quinazoline-2,4-dione;
1-Cyclopropyl-5-difluoromethyl-7-(4,5-dihydroxyoctahydrocyclohepta[c]pyrrol-2-yl)-6-fluoro-8-methoxy-1H-quinazoline-2,4-dione;
1-Cyclopropyl-5-difluoromethyl-7-(4,5-dihydroxydecahydrocycloocta[c]pyrrol-2-yl)-6-fluoro-8-methoxy-1H-quinazoline-2,4-dione;
1-Cyclopropyl-6-fluoro-7-(3-fluoro-4-hydroxymethylpyrrolidin-1-yl)-8-methoxy-5-methyl-1H-quinazoline-2,4-dione;
1-Cyclopropyl-7-[3-(1,2-dihydroxyethyl)-4-fluoropyrrolidin-1-yl]-6-fluoro-8-methoxy-5-methyl-1H-quinazoline-2,4-dione;
1-Cyclopropyl-6-fluoro-8-methoxy-5-methyl-7-[3-(2,2,2-trifluoro-1-hydroxyethyl)pyrrolidin-1-yl]-1H-quinazoline-2,4-dione;
1-Cyclopropyl-7-(3,4-dihydroxypiperidin-1-yl)-6-fluoro-8-methoxy-5-methyl-1H-quinazoline-2,4-dione;
1-Cyclopropyl-6-fluoro-7-(4-hydroxy-3-hydroxymethylpiperidin-1-yl)-8-methoxy-5-methyl-1H-quinazoline-2,4-dione;
1-Cyclopropyl-6-fluoro-7-(3-hydroxy-4-methylpiperidin-1-yl)-8-methoxy-5-methyl-1H-quinazoline-2,4-dione;
1-Cyclopropyl-6-fluoro-7-(3-hydroxymethyl-4-methylpiperidin-1-yl)-8-methoxy-5-methyl-1H-quinazoline-2,4-dione;
1-Cyclopropyl-7-(4-ethyl-3-hydroxymethylpiperidin-1-yl)-6-fluoro-8-methoxy-5-methyl-1H-quinazoline-2,4-dione;
1-Cyclopropyl-7-(4-ethyl-3-hydroxypiperidin-1-yl)-6-fluoro-8-methoxy-5-methyl-1H-quinazoline-2,4-dione;
1-Cyclopropyl-7-[3-(1,2-dihydroxy-2-methylpropyl)-4-fluoropyrrolidin-1-yl]-6-fluoro-8-methoxy-5-methyl-1H-quinazoline-2,4-dione;
1-Cyclopropyl-6-fluoro-7-[3-fluoro-4-(3,3,3-trifluoro-1,2-dihydroxy-2-trifluoromethylpropyl)-pyrrolidin-1-yl]-8-methoxy-5-methyl-1H-quinazoline-2,4-dione;
1-Cyclopropyl-6-fluoro-7-{3-fluoro-4-[hydroxy-(1-hydroxycyclopropyl)methyl]-pyrrolidin-1-yl}-8-methoxy-5-methyl-1H-quinazoline-2,4-dione;
1-Cyclopropyl-6-fluoro-7-{3-fluoro-4-[hydroxy-(1-hydroxycyclopentyl)methyl]pyrrolidin-1-yl}-8-methoxy-5-methyl-1H-quinazoline-2,4-dione;
5-Amino-1-cyclopropyl-6-fluoro-7-(3-fluoro-4-hydroxymethylpyrrolidin-1-yl)-8-methoxy-1H-quinazoline-2,4-dione;
5-Amino-1-cyclopropyl-7-[3-(1,2-dihydroxyethyl)-4-fluoropyrrolidin-1-yl]-6-fluoro-8-methoxy-1H-quinazoline-2,4-dione;
5-Amino-1-cyclopropyl-6-fluoro-8-methoxy-7-[3-(2,2,2-trifluoro-1-hydroxyethyl)pyrrolidin-1-yl]-1H-quinazoline-2,4-dione;
5-Amino-1-cyclopropyl-7-(3,4-dihydroxypiperidin-1-yl)-6-fluoro-8-methoxy-1H-quinazoline-2,4-dione;
5-Amino-1-cyclopropyl-6-fluoro-7-(4-hydroxy-3-hydroxymethylpiperidin-1-yl)-8-methoxy-1H-quinazoline-2,4-dione;
5-Amino-1-cyclopropyl-6-fluoro-7-(3-hydroxy-4-methylpiperidin-1-yl)-8-methoxy-1H-quinazoline-2,4-dione;
5-Amino-1-cyclopropyl-6-fluoro-7-(3-hydroxymethyl-4-methylpiperidin-1-yl)-8-methoxy-1H-quinazoline-2,4-dione;
5-Amino-1-cyclopropyl-7-(4-ethyl-3-hydroxymethylpiperidin-1-yl)-6-fluoro-8-methoxy-1H-quinazoline-2,4-dione;
5-Amino-1-cyclopropyl-7-(4-ethyl-3-hydroxypiperidin-1-yl)-6-fluoro-8-methoxy-1H-quinazoline-2,4-dione;
5-Amino-1-cyclopropyl-7-[3-(1,2-dihydroxy-2-methylpropyl)-4-fluoropyrrolidin-1-yl]-6-fluoro-8-methoxy-1H-quinazoline-2,4-dione;
5-Amino-1-cyclopropyl-6-fluoro-7-[3-fluoro-4-(3,3,3-trifluoro-1,2-dihydroxy-2-trifluoromethylpropyl)pyrrolidin-1-yl]-8-methoxy-1H-quinazoline-2,4-dione;
5-Amino-1-cyclopropyl-6-fluoro-7-{3-fluoro-4-[hydroxy-(1-hydroxycyclopropyl)methyl]pyrrolidin-1-yl}-8-methoxy-1H-quinazoline-2,4-dione;
5-Amino-1-cyclopropyl-6-fluoro-7-{3-fluoro-4-[hydroxy-(1-hydroxycyclopentyl)methyl]pyrrolidin-1-yl}-8-methoxy-1H-quinazoline-2,4-dione;
1-Cyclopropyl-5-difluoromethyl-6-fluoro-7-(3-fluoro-4-hydroxymethylpyrrolidin-1-yl)-8-methoxy-1H-quinazoline-2,4-dione;
1-Cyclopropyl-5-difluoromethyl-7-[3-(1,2-dihydroxyethyl)-4-fluoropyrrolidin-1-yl]-6-fluoro-8-methoxy-1 H-quinazoline-2,4-dione;
1-Cyclopropyl-5-difluoromethyl-6-fluoro-8-methoxy-7-[3-(2,2,2-trifluoro-1-hydroxyethyl)pyrrolidin-1-yl]-1H-quinazoline-2,4-dione;
1-Cyclopropyl-5-difluoromethyl-7-(3,4-dihydroxypiperidin-1-yl)-6-fluoro-8-methoxy-1H-quinazoline-2,4-dione;
1-Cyclopropyl-5-difluoromethyl-6-fluoro-7-(4-hydroxy-3-hydroxymethylpiperidin-1-yl)-8-methoxy-1H-quinazoline-2,4-dione;
1-Cyclopropyl-5-difluoromethyl-6-fluoro-7-(3-hydroxy-4-methylpiperidin-1-yl)-8-methoxy-1H-quinazoline-2,4-dione;
1-Cyclopropyl-5-difluoromethyl-6-fluoro-7-(3-hydroxymethyl-4-methylpiperidin-1-yl)-8-methoxy-1H-quinazoline-2,4-dione;
1-Cyclopropyl-5-difluoromethyl-7-(4-ethyl-3-hydroxymethylpiperidin-1-yl)-6-fluoro-8-methoxy-1H-quinazoline-2,4-dione;
1-Cyclopropyl-5-difluoromethyl-7-(4-ethyl-3-hydroxypiperidin-1-yl)-6-fluoro-8-methoxy-1H-quinazoline-2,4-dione;
1-Cyclopropyl-5-difluoromethyl-7-[3-(1,2-dihydroxy-2-methylpropyl)-4-fluoropyrrolidin-1-yl]-6-fluoro-8-methoxy-1H-quinazoline-2,4-dione;
1-Cyclopropyl-5-difluoromethyl-6-fluoro-7-[3-fluoro-4-(3,3,3-trifluoro-1,2-dihydroxy-2-trifluoromethylpropyl)pyrrolidin-1-yl]-8-methoxy-1H-quinazoline-2,4-dione;
1-Cyclopropyl-5-difluoromethyl-6-fluoro-7-{3-fluoro-4-[hydroxy-(1-hydroxycyclopropyl)methyl]pyrrolidin-1-yl}-8-methoxy-1H-quinazoline-2,4-dione;
1-Cyclopropyl-5-difluoromethyl-6-fluoro-7-{3-fluoro-4-[hydroxy-(1-hydroxycyclopentyl)methyl]pyrrolidin-1-yl}-8-methoxy-1H-quinazoline-2,4-dione; or
a pharmaceutically acceptable salt thereof.

53. A pharmaceutical composition comprising a compound of any of claims 1, 7, 14, 20, 26, 34, 40, 46, or 52 admixed with a carrier, diluent, or excipient.

54. A method of treating a bacterial infection in a mammal comprising administering to the mammal in need thereof an antibacterial effective amount of a compound of any of claims 1, 7, 14, 20, 26, 34, 40, 46, or 52.

55. A method of inhibiting a bacterial topoisomerase in a mammal comprising administering to the mammal in need thereof an effective amount of a compound of any of claims 1, 7, 14, 20, 26, 34, 40, 46, or 52.

56. A method of inhibiting a bacterial DNA gyrase in a mammal comprising administering to the mammal in need thereof an effective amount of a compound of any of claims 1, 7, 14, 20, 26, 34, 40, 46, or 52.

57. A method of inhibiting a bacterial topoisomerase IV in a mammal comprising administering to the mammal in need thereof an effective amount of a compound of any of claims 1, 7, 14, 20, 26, 34, 40, 46, or 52.

58. A method of inhibiting a quinolone resistant bacteria in a mammal comprising administering to the mammal an effective amount of a compound of any of claims 1, 7, 14, 20, 26, 34, 40, 46, or 52.

59. A method of inhibiting a quinolone resistant bacterial DNA gyrase in a mammal comprising administering to the mammal an effective amount of a compound of any of claims 1, 7, 14, 20, 26, 34, 40, 46, or 52.

60. A method of inhibiting a quinolone resistant bacterial topoisomerase in a mammal comprising administering to the mammal an effective amount of a compound of any of claims 1, 7, 14, 20, 26, 34, 40, 46, or 52.

61. A process for preparing a compound of formula IX, wherein R₁, R₂, R₃, R₄, R₆, J, K, Z, Z′, z, and R′ are as defined above and R_{5′} is halo, comprising:
(a) coupling compound IXA wherein R₅ is halo with compound IXB wherein M is n-Bu₃Sn in the presence of Pd° to provide the R5-coupled product IXC; 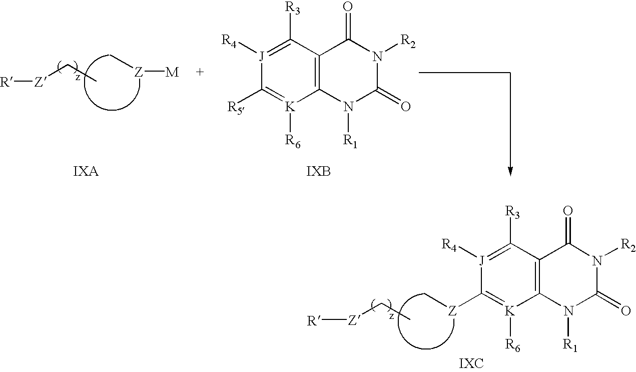
and
(b) removing the R′ group in IXC to provide compound IXD [chemical structure]
